(19) United States
(12) Patent Application Publication
(10) Pub. No.: US 2024/0316038 A1
(43) Pub. Date: Sep. 26, 2024

(54) APOL1 INHIBITORS AND METHODS OF USE

(71) Applicant: Maze Therapeutics, Inc., South San Francisco, CA (US)

(72) Inventors: Patrick Sang Tae LEE, Walnut Creek, CA (US); Todd Jonathan August EWING, Walnut Creek, CA (US); Adam Neil REID, San Francisco, CA (US); Christopher Joseph SINZ, Walnut Creek, CA (US); Birong ZHANG, Union City, CA (US); Sarah M. BRONNER, Oakland, CA (US); David John MORGANS, Jr., Los Altos, CA (US); Maarten HOEK, Belmont, CA (US); Victoria Anne ASSIMON, San Francisco, CA (US)

(21) Appl. No.: 18/277,943

(22) PCT Filed: Feb. 18, 2022
(86) PCT No.: PCT/US2022/017086

§ 371 Date: Aug. 18, 2023

Related U.S. Application Data
(60) Provisional application No. 63/151,605, filed on Feb. 19, 2021.

(51) Int. Cl. A61K 31/496; A61K 31/145; A61K 31/341; A61K 31/4025; A61K 31/44; A61K 31/5375; A61K 31/554; A61P 13/12; C07C 311/08; C07C 317/22; C07D 207/08; C07D 211/22; C07D 211/42; C07D 225/02; C07D 265/30; C07D 267/10; C07D 295/027; C07D 307/22; C07D 403/12

(52) U.S. Cl. CPC A61K 31/496; A61K 31/145; A61K 31/341; A61K 31/4025; A61K 31/44; A61K 31/5375; A61K 31/554; A61P 13/12; C07C 311/08; C07C 317/22; C07D 207/08; C07D 211/22; C07D 211/42; C07D 225/02; C07D 265/30; C07D 267/10; C07D 295/027; C07D 307/22; C07D 403/12

(57) ABSTRACT

Provided herein are compounds of formula (A′): or a stereoisomer or tautomer thereof, or a pharmaceutically acceptable salt of any of the foregoing, wherein X¹, X², X³, X⁴, R^{a}, R^{b}, R^{c}, L, Q, and Y are as defined herein. Also provided are methods of N inhibiting APOL1 and methods of preparing compounds of formula (A′). Also provided are methods of inhibiting APOL1 and methods of treating an APOL1-mediated disease, disorder, or condition in an individual.

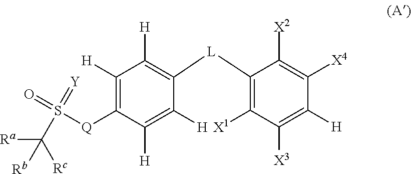

### CROSS REFERENCE TO RELATED APPLICATIONS

This application claims priority to U.S. Provisional Application No. 63/151,605 filed on Feb. 19, 2021, the content of which is incorporated herein by reference in its entirety.

### BACKGROUND OF THE INVENTION

Apolipoprotein L1 (APOL1) is a pore forming innate immunity factor, protecting individuals from trypanosome parasites (Vanhamme, L. et al. Nature (2003) 422, 83-87). The secreted form of APOL1 circulates in blood as part of distinct high-density lipoprotein (HDL) complexes, known as trypanosome lytic factors (TLFs) (Rifkin, M. R. Proc. Natl. Acad. Sci. USA. (1978) 75, 3450-3454; Raper, J. et al. Infect. Immun. (1999) 67, 1910-1916). TLFs are internalized by the parasites through endocytosis (Hager, K. M. et al. J. Cell Biol. (1994) 126, 155-167). Within trypanosomes, APOL1 forms cation pores, causing ion flux, swelling, and eventual lysis (Rifkin, M. R. Exp. Parasitol. (1984) 58, 81-93; Molina-Portela, M. P. et al. Mol. Biochem. Parasitol. (2005) 144, 218-226; Pérez-Morga, D. et al. Science. (2005) 309, 469-472; Thomson, R. & Finkelstein, A. Proc. Natl. Acad. Sci. USA. (2015) 112, 2894-2899).

Several Trypanosoma brucei subspecies (T.b. rhodesiense and T.b. gambiense) developed resistance mechanisms to APOL1-dependent killing (Pays, E. et al. Nat. Rev. Microbiol. (2014) 12, 575-584). Positive selection resulted in APOL1 variants, G1 (S342G, I384M) and G2 (N388A, Y389A), capable of interfering with these resistance mechanisms (Genovese, G. et al. Science. (2010) 329, 841-845). However, individuals with any binary combination of these variants (G1/G1, G2/G2, or G1/G2), have a greater risk of developing a variety of chronic kidney diseases, including focal segmental glomerulosclerosis (FSGS), hypertension-attributed kidney disease, human immunodeficiency virus-associated nephropathy (HIVAN) (Genovese, G. et al. Science. (2010) 329, 841-845; Tzur, S. et al. Hum. Genet. (2010) 128, 345-350; Kopp, J. B. et al. J. Am. Soc. Nephrol. (2011) 22, 2129-2137), sickle cell nephropathy (Ashley-Koch, A. E. et al. Br. J. Haematol. (2011) 155, 386-394), lupus nephritis (Freedman, B. I. et al. Arthritis Rheumatol. (2014) 66, 390-396), and an increased rate of Glomerular Filtration Rate (GFR) decline in diabetic kidney disease (Parsa, A. et al. N. Engl. J. Med. (2013) 369, 2183-2196). The APOL1 high-risk genotype has also been associated with COVID-19 associated nephropathy and other viral nephropathies (Shetty, A. et al. J. Am. Soc. Nephrol. (2021) 32, 33-40; Chang, J. H. et al. Am. J. Kidney Dis. (2019) 73, 134-139). Moreover, decreased renal allograft survival has been observed after deceased-donor kidney transplantations from APOL1 high-risk genotype donors (Freedman, B. I. et al. Transplantation. (2016) 100, 194-202). In addition, having two APOL1 risk alleles increases risk for preeclampsia (Reidy, K. J. et al. Am. J. Hum. Genet. (2018) 103, 367-376) and sepsis (Chaudhary, N. S. et al. Clin. J. Am. Soc. Nephrol. (2019) 14, 1733-1740). There are no approved therapies for APOL1-associated nephropathy, and patients are treated based on the standard of care for their underlying form of chronic kidney disease. This presents a clear unmet need for therapies targeted to people with the APOL1 high-risk genotype.

Numerous studies have shown that APOL1 risk variants are toxic when overexpressed in human cells (Wan, G. et al. J. Biol. Chem. (2008) 283, 21540-21549; Lan, X. et al. Am. J. Physiol. Renal Physiol. (2014) 307, F326-F336; Olabisi, O. A. et al. Proc. Natl. Acad. Sci. USA. (2016) 113, 830-837; Ma, L. et al. J. Am. Soc. Nephrol. (2017) 28, 1093-1105; Lannon, H. et al. Kidney Int. (2019) 96, 1303-1307). Recent findings suggest that this toxicity is associated with APOL1 pore function (Giovinazzo, J. A. et al. eLife. (2020) 9, e51185). Thus, there is a need to develop compounds suitable for inhibiting APOL1 activity and methods for inhibiting the activity of APOL1 using such compounds.

### BRIEF SUMMARY OF THE INVENTION

This disclosure describes compounds and compositions that may be useful for the treatment of APOL1-mediated diseases, including a variety of chronic kidney diseases such as FSGS, hypertension-attributed kidney disease, HIVAN, sickle cell nephropathy, lupus nephritis, diabetic kidney disease, viral nephropathy, COVID-19 associated nephropathy, and APOL1-associated nephropathy. The compounds and compositions may also find use in treating other APOL1-mediated disorders such as preeclampsia and sepsis. Additionally, for individuals with the APOL1 high-risk genotype, the disclosed compounds and compositions could have utility in preventing the onset of non-diabetic renal disease and/or delaying the progression of any form of chronic kidney disease. The disclosed chemical matter could also have utility in preventing and/or delaying progressive renal allograft loss in patients who have received a kidney transplant from a high-risk APOL1 genotype donor.

In one aspect, provided herein is a compound of formula (A′):

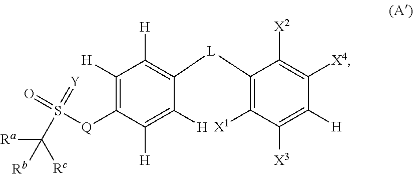

or a stereoisomer or tautomer thereof, or a pharmaceutically acceptable salt of any of the foregoing, wherein:
Q is absent or is —N—(C₁₋₆alkyl);
Y is O or —N—(C₁₋₆alkyl),
   provided that, when Q is —N(C₁₋₆alkyl), then Y is O;
R^{a}, R^{b}, and R^{c} are each independently H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl,
or any two of R^{a}, R^{b}, and R^{c} are taken, together with the atoms to which they are attached, to form a C₃₋₆cycloalkyl or a 3-6 membered heterocyclyl, and the other of R^{a}, R^{b}, and R^{c} is H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl;
L is selected from the group consisting of:

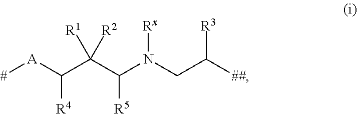

A is O, NH, N(C₁₋₆alkyl), CH₂, or CH(C₁₋₆alkyl);
R^{x} is H,
or R^{x} is taken together with one of R¹ and R², and the atoms to which they are attached, to form a 3-8 membered heterocyclyl, wherein the 3-8 membered heterocyclyl is substituted with n independently selected R⁹ substituents, wherein n is an integer from 0-6, and R^{g} is —OH, halo, C₁₋₆alkyl, or C₁₋₆alkoxy;
R¹ and R² are independently H, halo, or —OH,
or one of R¹ and R² is taken together with R^{x}, and the atoms to which they are attached, to form a 3-8 membered heterocyclyl, wherein the 3-8 membered heterocyclyl is substituted with n independently selected R⁹ substituents, wherein n is an integer from 0-6, and R^{g} is —OH, halo, C₁₋₆alkyl, or C₁₋₆alkoxy, and the other of R¹ and R² is H, halo, or —OH;
R³ is H, —OH, halo, or C₁₋₆alkoxy; and
R⁴ and R⁵ are independently H,
or R⁴ and R⁵ are taken, together with the atoms to which they are attached, to form a C₃₋₈cycloalkyl,
provided that either:
   (1) R^{x} is taken together with one of R¹ and R², and the atoms to which they are attached, to form a 3-8 membered heterocyclyl, wherein the 3-8 membered heterocyclyl is optionally substituted with one or more —OH, C₁₋₆alkyl, or C₁₋₆alkoxy, or
   (2) R⁴ and R⁵ are taken, together with the atoms to which they are attached, to form a C₃₋₈cycloalkyl,

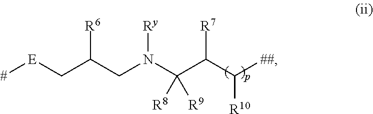

wherein
E is O, NH, N(C₁₋₆alkyl), CH₂, or CH(C₁₋₆alkyl);
p is 0 or 1,
   provided that, when p is 1, then E is O;
R⁶ is H or —OH;
R^{y} is H,
or R^{y} is taken together with R⁷, and the atoms to which they are attached, to form a 3-8 membered heterocyclyl,
or R^{y} is taken together with one of R⁸ and R⁹, and the atoms to which they are attached, to form a 3-8 membered heterocyclyl;
R⁷ is H,
or R⁷ is taken together with R^{y}, and the atoms to which they are attached, to form a 3-8 membered heterocyclyl;
R⁸ and R⁹ are independently H or C₁₋₆alkyl,
or one of R⁸ and R⁹ is taken together with R^{y}, and the atoms to which they are attached, to form a 3-8 membered heterocyclyl, and the other of R⁸ and R⁹ is H or C₁₋₆alkyl,
or one of R⁸ and R⁹ is taken together with R¹⁰, and the atoms to which they are attached, to form a C₃₋₈cycloalkyl, and the other of R⁸ and R⁹ is H or C₁₋₆alkyl; and
R¹⁰ is H,
or R¹⁰ is taken together with one of R⁸ and R⁹, and the atoms to which they are attached, to form a C₃₋₈cycloalkyl,
provided that:
   (1) R^{y} is taken together with R⁷, and the atoms to which they are attached, to form a 3-8 membered heterocyclyl, or
   (2) R^{y} is taken together with one of R⁸ and R⁹, and the atoms to which they are attached, to form a 3-8 membered heterocyclyl, or
   (3) one of R⁸ and R⁹ is taken together with R¹⁰ and the atoms to which they are attached, to form a C₃₋₈cycloalkyl, and

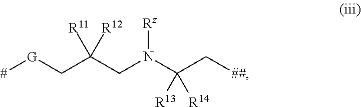

wherein
G is O, NH, N(C₁₋₆alkyl), CH₂, or CH(C₁₋₆alkyl);
R^{z} is H or C₁₋₆alkyl, wherein the C₁₋₆alkyl is optionally substituted with one or more C₃₋₈cycloalkyl;
R¹¹ and R¹² are independently H, —OH, halo, or C₁₋₆alkyl; and
R¹³ and R¹⁴ are independently H, C₁₋₆alkyl, or C₃₋₈cycloalkyl,
or R¹³ and R¹⁴ are taken, together with the atoms to which they are attached, to form a 3-8 membered heterocyclyl,
wherein, for each of (i)-(iii), #denotes the point of attachment to the phenyl ring bearing moiety Q, and ##denotes the point of attachment to the phenyl ring bearing moieties X¹-X⁴; and
X¹, X², X³, and X⁴ are, independently of each other, H, halo, —CN, C₁₋₆alkyl, C₁₋₆alkoxy, or SF₅ wherein the C₁₋₆alkyl or C₁₋₆alkoxy is optionally substituted with one or more halo,
   provided that at least one of X¹, X², X³, and X⁴ is halo, —CN, C₁₋₆alkyl, C₁₋₆alkoxy, or SF₅, wherein the C₁₋₆alkyl or C₁₋₆alkoxy is optionally substituted with one or more halo.

In one aspect, provided herein is a compound of formula (A):

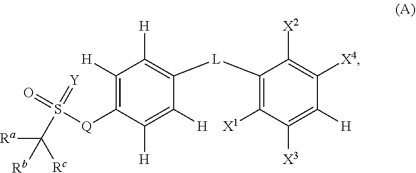

or a stereoisomer or tautomer thereof, or a pharmaceutically acceptable salt of any of the foregoing, wherein:
Q is absent or is —N—(C₁₋₆alkyl);
Y is O or —N—(C₁₋₆alkyl),
   provided that, when Q is —N(C₁₋₆alkyl), then Y is O;
R^{a}, R^{b}, and R^{c} are each independently H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl,
or any two of R^{a}, R^{b}, and R^{c} are taken, together with the atoms to which they are attached, to form a C₃₋₆cycloalkyl or a 3-6 membered heterocyclyl, and the other of R^{a}, R^{b}, and R^{c} is H or C₁₋₆alkyl, wherein the C₁₋₆alkyl of R^{a}, R^{b}, or R^{c} is independently optionally substituted with one or more —OH, C₁₋₆alkoxy, or —S(O)₂—C₁₋₆alkyl;
L is selected from the group consisting of:

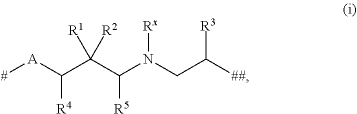

wherein
   A is O, NH, N(C₁₋₆alkyl), CH₂, or CH(C₁₋₆alkyl);
   R^{x} is H,
   or R^{x} is taken together with one of R¹ and R², and the atoms to which they are attached, to form a 3-8 membered heterocyclyl, wherein the 3-8 membered heterocyclyl is substituted with n independently selected R^{g} substituents, wherein n is an integer from 0-6, and R^{g} is —OH, halo, C₁₋₆alkyl, or C₁₋₆alkoxy;
   R¹ and R² are independently H, halo, or —OH,
   or one of R¹ and R² is taken together with R^{x}, and the atoms to which they are attached, to form a 3-8 membered heterocyclyl, wherein the 3-8 membered heterocyclyl is substituted with n independently selected R^{g} substituents, wherein n is an integer from 0-6, and R^{g} is —OH, halo, C₁₋₆alkyl, or C₁₋₆alkoxy, and the other of R¹ and R² is H, halo, or —OH;
   R³ is H, —OH, halo, or C₁₋₆alkoxy; and
   R⁴ and R⁵ are independently H,
   or R⁴ and R⁵ are taken, together with the atoms to which they are attached, to form a C₃₋₈cycloalkyl,
   provided that either:
      (1) R^{x} is taken together with one of R¹ and R², and the atoms to which they are attached, to form a 3-8 membered heterocyclyl, wherein the 3-8 membered heterocyclyl is optionally substituted with one or more —OH, C₁₋₆alkyl, or C₁₋₆alkoxy, or
      (2) R⁴ and R⁵ are taken, together with the atoms to which they are attached, to form a C₃₋₈cycloalkyl,

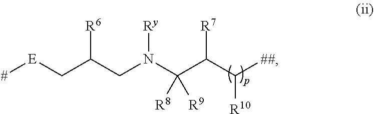

wherein
E is O, NH, N(C₁₋₆alkyl), CH₂, or CH(C₁₋₆alkyl);
p is 0 or 1,
   provided that, when p is 1, then E is O;
R⁶ is H or —OH;
R^{y} is H,
or R^{y} is taken together with R⁷, and the atoms to which they are attached, to form a 3-8 membered heterocyclyl,
or R^{y} is taken together with one of R⁸ and R⁹, and the atoms to which they are attached, to form a 3-8 membered heterocyclyl;
R⁷ is H,
or R⁷ is taken together with R^{y}, and the atoms to which they are attached, to form a 3-8 membered heterocyclyl;
R⁸ and R⁹ are independently H or C₁₋₆alkyl,
or one of R⁸ and R⁹ is taken together with R^{y}, and the atoms to which they are attached, to form a 3-8 membered heterocyclyl, and the other of R⁸ and R⁹ is H or C₁₋₆alkyl,
or one of R⁸ and R⁹ is taken together with R¹⁰, and the atoms to which they are attached, to form a C₃₋₈cycloalkyl, and the other of R⁸ and R⁹ is H or C₁₋₆alkyl; and
R¹⁰ is H,
or R¹⁰ is taken together with one of R⁸ and R⁹, and the atoms to which they are attached, to form a C₃₋₈cycloalkyl,
provided that:
   (1) R^{y} is taken together with R⁷, and the atoms to which they are attached, to form a 3-8 membered heterocyclyl, or
   (2) R^{y} is taken together with one of R⁸ and R⁹, and the atoms to which they are attached, to form a 3-8 membered heterocyclyl, or
   (3) one of R⁸ and R⁹ is taken together with R¹⁰ and the atoms to which they are attached, to form a C₃₋₈cycloalkyl, and

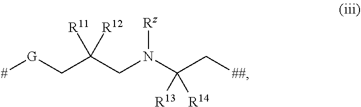

wherein
G is O, NH, N(C₁₋₆alkyl), CH₂, or CH(C₁₋₆alkyl);
   R^{z} is H or C₁₋₆alkyl, wherein the C₁₋₆alkyl is optionally substituted with one or more C₃₋₈cycloalkyl;
   R¹¹ and R¹² are independently H, —OH, halo, or C₁₋₆alkyl; and
   R¹³ and R¹⁴ are independently H, C₁₋₆alkyl, or C₃₋₈cycloalkyl,
   or R¹³ and R¹⁴ are taken, together with the atoms to which they are attached, to form a 3-8 membered heterocyclyl,
wherein, for each of (i)-(iii), #denotes the point of attachment to the phenyl ring bearing moiety Q, and ##denotes the point of attachment to the phenyl ring bearing moieties X¹-X⁴; and
X¹, X², X³, and X⁴ are, independently of each other, H, halo, —CN, C₁₋₆alkyl, or C₁₋₆alkoxy, wherein the C₁₋₆alkyl or C₁₋₆alkoxy is optionally substituted with one or more halo,
   provided that at least one of X¹, X², X³, and X⁴ is halo, —CN, C₁₋₆alkyl, or C₁₋₆alkoxy, wherein the C₁₋₆alkyl or C₁₋₆alkoxy is optionally substituted with one or more halo.

Any embodiments provided herein of a compound of formula (A), or a stereoisomer or tautomer thereof, or a pharmaceutically acceptable salt of any of the foregoing, or any variation or embodiment thereof, are also embodiments of a compound of formula (A′), or a stereoisomer or tautomer thereof, or a pharmaceutically acceptable salt of any of the foregoing, or any variation or embodiment thereof.

In one aspect, provided herein is a compound of formula (I):

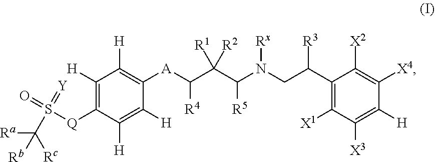

or a stereoisomer or tautomer thereof, or a pharmaceutically acceptable salt of any of the foregoing, wherein X¹, X², X³, X⁴, R¹, R², R³, R⁴, R⁵, R^{a}, R^{b}, R^{c}, R^{x}, A, Q, and Y are as defined for a compound of formula (A), or a stereoisomer or tautomer thereof, or a pharmaceutically acceptable salt of any of the foregoing, or any variation or embodiment thereof. In another variation, X¹, X², X³, X⁴, R¹, R², R³, R⁴, R⁵, R^{a}, R^{b}, R^{c}, R^{x}, A, Q, and Y of formula (I) are as defined for a compound of formula (A′), or a stereoisomer or tautomer thereof, or a pharmaceutically acceptable salt of any of the foregoing, or any variation or embodiment thereof.

In one aspect, provided herein is a compound of formula (II):

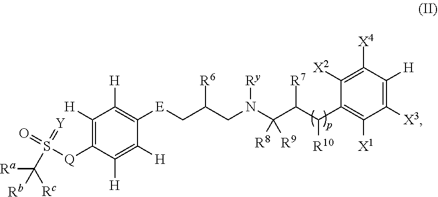

or a stereoisomer or tautomer thereof, or a pharmaceutically acceptable salt of any of the foregoing, wherein X¹, X², X³, X⁴, R⁶, R⁷, R⁸, R⁹, R¹⁰, R^{a}, R^{b}, R^{c}, R^{y}, p, E, Q, and Y are as defined for a compound of formula (A), or a stereoisomer or tautomer thereof, or a pharmaceutically acceptable salt of any of the foregoing, or any variation or embodiment thereof. In another variation, X¹, X², X³, X⁴, R⁶, R⁷, R⁸, R⁹, R¹⁰, R^{a}, R^{b}, R^{c}, R^{y}, p, E, Q, and Y of formula (II) are as defined for a compound of formula (A′), or a stereoisomer or tautomer thereof, or a pharmaceutically acceptable salt of any of the foregoing, or any variation or embodiment thereof. In one aspect, provided herein is a compound of formula (III):

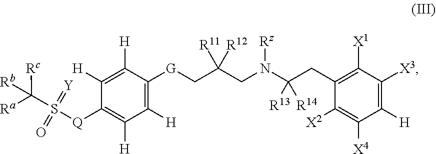

or a stereoisomer or tautomer thereof, or a pharmaceutically acceptable salt of any of the foregoing, wherein X¹, X², X³, X⁴, R¹¹, R¹², R¹³, R¹⁴, R^{a}, R^{b}, R^{c}, R^{z}, G, Q, and Y are as defined for a compound of formula (A), or a stereoisomer or tautomer thereof, or a pharmaceutically acceptable salt of any of the foregoing, or any variation or embodiment thereof. In another variation, X¹, X², X³, X⁴, R¹¹, R¹², R¹³, R¹⁴, R^{a}, R^{b}, R^{c}, R^{z}, G, Q, and Y of formula (III) are as defined for a compound of formula (A′), or a stereoisomer or tautomer thereof, or a pharmaceutically acceptable salt of any of the foregoing, or any variation or embodiment thereof.

In one aspect, provided herein is a compound of formula (B-2):

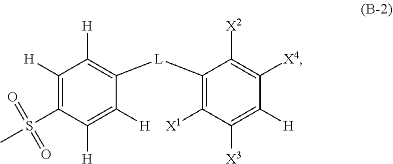

or a stereoisomer or tautomer thereof, or a pharmaceutically acceptable salt of any of the foregoing, wherein X¹, X², X³, X⁴, and L are as defined for a compound of formula (A), or a stereoisomer or tautomer thereof, or a pharmaceutically acceptable salt of any of the foregoing, or any variation or embodiment thereof. In another variation, X¹, X², X³, X⁴, and L of formula (B-2) are as defined for a compound of formula (A′), or a stereoisomer or tautomer thereof, or a pharmaceutically acceptable salt of any of the foregoing, or any variation or embodiment thereof.

In one aspect, provided herein is a compound of formula (B-5):

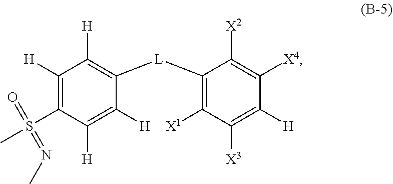

or a stereoisomer or tautomer thereof, or a pharmaceutically acceptable salt of any of the foregoing, wherein X¹, X², X³, X⁴, and L are as defined for a compound of formula (A), or a stereoisomer or tautomer thereof, or a pharmaceutically acceptable salt of any of the foregoing, or any variation or embodiment thereof. In another variation, X¹, X², X³, X⁴, and L of formula (B-5) are as defined for a compound of formula (A′), or a stereoisomer or tautomer thereof, or a pharmaceutically acceptable salt of any of the foregoing, or any variation or embodiment thereof.

In one aspect, provided herein is a compound of formula (C-1):

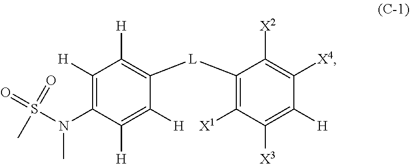

or a stereoisomer or tautomer thereof, or a pharmaceutically acceptable salt of any of the foregoing, wherein X¹, X², X³, X⁴, and L are as defined for a compound of formula (A), or a stereoisomer or tautomer thereof, or a pharmaceutically acceptable salt of any of the foregoing, or any variation or embodiment thereof. In another variation, X¹, X², X³, X⁴, and L of formula (C-1) are as defined for a compound of formula (A′), or a stereoisomer or tautomer thereof, or a pharmaceutically acceptable salt of any of the foregoing, or any variation or embodiment thereof.

In one aspect, provided herein is a pharmaceutical composition, comprising (i) a compound of formula (A), or any embodiment or variation thereof, such as a compound of formula (I), (II), (III), (B-2), (B-5), or (C-1), or a stereoisomer or tautomer thereof, or a pharmaceutically acceptable salt of any of the foregoing, and (ii) one or more pharmaceutically acceptable excipients. In another variation, provided herein is a pharmaceutical composition, comprising (i) a compound of formula (A′), or any embodiment or variation thereof, such as a compound of formula (I), (II), (III), (B-2), (B-5), or (C-1), or a stereoisomer or tautomer thereof, or a pharmaceutically acceptable salt of any of the foregoing, and (ii) one or more pharmaceutically acceptable excipients.

In one aspect, provided herein is a method of modulating APOL1 in a cell, comprising exposing the cell to a composition comprising an effective amount of a compound of formula (A), or any embodiment or variation thereof, such as a compound of formula (I), (II), (III), (B-2), (B-5), or (C-1), or a stereoisomer or tautomer thereof, or a pharmaceutically acceptable salt of any of the foregoing, or a pharmaceutical composition comprising (i) a compound of formula (A), or any embodiment or variation thereof, such as a compound of formula (I), (II), (III), (B-2), (B-5), or (C-1), or a stereoisomer or tautomer thereof, or a pharmaceutically acceptable salt of any of the foregoing, and (ii) one or more pharmaceutically acceptable excipients. In another variation, provided herein is a method of modulating APOL1 in a cell, comprising exposing the cell to a composition comprising an effective amount of a compound of formula (A′), or any embodiment or variation thereof, such as a compound of formula (I), (II), (III), (B-2), (B-5), or (C-1), or a stereoisomer or tautomer thereof, or a pharmaceutically acceptable salt of any of the foregoing, or a pharmaceutical composition comprising (i) a compound of formula (A′), or any embodiment or variation thereof, such as a compound of formula (I), (II), (III), (B-2), (B-5), or (C-1), or a stereoisomer or tautomer thereof, or a pharmaceutically acceptable salt of any of the foregoing, and (ii) one or more pharmaceutically acceptable excipients.

In one aspect, provided herein is a method of inhibiting APOL1 in a cell, comprising exposing the cell to a composition comprising an effective amount of a compound of formula (A), or any embodiment or variation thereof, such as a compound of formula (I), (II), (III), (B-2), (B-5), or (C-1), or a stereoisomer or tautomer thereof, or a pharmaceutically acceptable salt of any of the foregoing, or a pharmaceutical composition comprising (i) a compound of formula (A), or any embodiment or variation thereof, such as a compound of formula (I), (II), (III), (B-2), (B-5), or (C-1), or a stereoisomer or tautomer thereof, or a pharmaceutically acceptable salt of any of the foregoing, and (ii) one or more pharmaceutically acceptable excipients. In another variation, provided herein is a method of inhibiting APOL1 in a cell, comprising exposing the cell to a composition comprising an effective amount of a compound of formula (A′), or any embodiment or variation thereof, such as a compound of formula (I), (II), (III), (B-2), (B-5), or (C-1), or a stereoisomer or tautomer thereof, or a pharmaceutically acceptable salt of any of the foregoing, or a pharmaceutical composition comprising (i) a compound of formula (A′), or any embodiment or variation thereof, such as a compound of formula (I), (II), (III), (B-2), (B-5), or (C-1), or a stereoisomer or tautomer thereof, or a pharmaceutically acceptable salt of any of the foregoing, and (ii) one or more pharmaceutically acceptable excipients.

In one aspect, provided herein is a method of treating an APOL1-mediated disease, disorder, or condition in an individual in need thereof, comprising administering to the individual an effective amount of a compound of formula (A), or any embodiment or variation thereof, such as a compound of formula (I), (II), (III), (B-2), (B-5), or (C-1), or a stereoisomer or tautomer thereof, or a pharmaceutically acceptable salt of any of the foregoing, or a pharmaceutical composition comprising (i) a compound of formula (A), or any embodiment or variation thereof, such as a compound of formula (I), (II), (III), (B-2), (B-5), or (C-1), or a stereoisomer or tautomer thereof, or a pharmaceutically acceptable salt of any of the foregoing, and (ii) one or more pharmaceutically acceptable excipients. In another variation, provided herein is a method of treating an APOL1-mediated disease, disorder, or condition in an individual in need thereof, comprising administering to the individual an effective amount of a compound of formula (A′), or any embodiment or variation thereof, such as a compound of formula (I), (II), (III), (B-2), (B-5), or (C-1), or a stereoisomer or tautomer thereof, or a pharmaceutically acceptable salt of any of the foregoing, or a pharmaceutical composition comprising (i) a compound of formula (A′), or any embodiment or variation thereof, such as a compound of formula (I), (II), (III), (B-2), (B-5), or (C-1), or a stereoisomer or tautomer thereof, or a pharmaceutically acceptable salt of any of the foregoing, and (ii) one or more pharmaceutically acceptable excipients.

In one aspect, provided herein is a kit, comprising (i) a compound of formula (A), or any embodiment or variation thereof, such as a compound of formula (I), (II), (III), (B-2), (B-5), or (C-1), or a stereoisomer or tautomer thereof, or a pharmaceutically acceptable salt of any of the foregoing, and (ii) instructions for use in treating an APOL1-mediated disease, disorder, or condition in an individual in need thereof. In another variation, provided herein is a kit, comprising (i) a compound of formula (A′), or any embodiment or variation thereof, such as a compound of formula (I), (II), (III), (B-2), (B-5), or (C-1), or a stereoisomer or tautomer thereof, or a pharmaceutically acceptable salt of any of the foregoing, and (ii) instructions for use in treating an APOL1-mediated disease, disorder, or condition in an individual in need thereof.

In some aspect, provided herein are methods of preparing a compound of formula (A) or (A′), or any embodiment or variation thereof, such as a compound of formula (I), (II), (III), (B-2), (B-5), or (C-1), or a stereoisomer or tautomer thereof, or a pharmaceutically acceptable salt of any of the foregoing.

### DETAILED DESCRIPTION OF THE INVENTION

Unless clearly indicated otherwise, the terms “a,” “an,” and the like, refer to one or more.

As used herein, “about” a parameter or value includes and describes that parameter or value per se. For example, “about X” includes and describes X per se.

“Individual” refers to mammals and includes humans and non-human mammals. Examples of individuals include, but are not limited to, some primates and humans. In some embodiments, individual refers to a human.

As used herein, an “at risk” individual is an individual who is at risk of developing a disease or condition. An individual “at risk” may or may not have a detectable disease or condition, and may or may not have displayed detectable disease prior to the treatment methods described herein. “At risk” denotes that an individual has one or more so-called risk factors, which are measurable parameters that correlate with development of a disease or condition and are known in the art. An individual having one or more of these risk factors has a higher probability of developing the disease or condition than an individual without these risk factor(s).

“Treatment” or “treating” is an approach for obtaining beneficial or desired results including clinical results. Beneficial or desired results may include one or more of the following: decreasing one or more symptom resulting from the disease or condition; diminishing the extent of the disease or condition; slowing or arresting the development of one or more symptom associated with the disease or condition (e.g., stabilizing the disease or condition, preventing or delaying the worsening or progression of the disease or condition); and relieving the disease, such as by causing the regression of clinical symptoms (e.g., ameliorating the disease state, enhancing the effect of another medication, delaying the progression of the disease, increasing the quality of life, and/or prolonging survival).

As used herein, “delaying” development of a disease or condition means to defer, hinder, slow, retard, stabilize and/or postpone development of the disease or condition. This delay can be of varying lengths of time, depending on the history of the disease and/or individual being treated. As is evident to one skilled in the art, a sufficient or significant delay can, in effect, encompass prevention, in that the individual does not develop the disease or condition.

As used herein, the term “therapeutically effective amount” or “effective amount” intends such amount of a compound of the disclosure or a pharmaceutically salt thereof sufficient to effect treatment when administered to an individual. As is understood in the art, an effective amount may be in one or more doses, e.g., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved.

As used herein, “unit dosage form” refers to physically discrete units, suitable as unit dosages, each unit containing a predetermined quantity of active ingredient, or compound, which may be in a pharmaceutically acceptable carrier.

As used herein, by “pharmaceutically acceptable” is meant a material that is not biologically or otherwise undesirable, e.g., the material may be incorporated into a pharmaceutical composition administered to an individual without causing significant undesirable biological effects.

The term “alkyl”, as used herein, refers to an unbranched or branched saturated hydrocarbon chain. As used herein, alkyl has 1-20 carbons (i.e., C₁₋₂₀alkyl), 1-16 carbons (i.e., C₁₋₆alkyl), 1-12 carbons (i.e., C₁₋₁₂alkyl), 1-10 carbons (i.e., C₁₋₁₀alkyl), 1-8 carbons (i.e., C₁₋₈alkyl), 1-6 carbons (i.e., C₁₋₆alkyl), 1-4 carbons (i.e., C₁₋₄alkyl), or 1-3 carbons (i.e., C₁₋₃alkyl). Examples of alkyl groups include, but are not limited to, methyl, ethyl, propyl, iso-propyl, n-butyl, sec-butyl, iso-butyl, tert-butyl, pentyl, 2-pentyl, iso-pentyl, neo-pentyl, hexyl, 2-hexyl, 3-hexyl, and 3-methylpentyl. When an alkyl residue having a specific number of carbons is named by chemical name or molecular formula, all positional isomers having that number of carbon atoms may be encompassed—for example, “butyl” includes n-butyl, sec-butyl, iso-butyl, and tert-butyl; and “propyl” includes n-propyl and iso-propyl. Certain commonly used alternative names may be used and will be understood by those of ordinary skill in the art. For instance, a divalent group, such as a divalent “alkyl” group, may be referred to as an “alkylene”.

The term “alkoxy”, as used herein, refers to an —O-alkyl moiety. Examples of alkoxy groups include, but are not limited to, methoxy, ethoxy, n-propoxy, iso-propoxy, n-butoxy, tert-butoxy, sec-butoxy, n-pentoxy, n-hexoxy, and 1,2-dimethylbutoxy.

The term “aryl”, as used herein, refers to a fully unsaturated carbocyclic ring moiety. The term “aryl” encompasses monocyclic and polycyclic fused-ring moieties. As used herein, aryl encompasses ring moieties comprising, for example, 6 to 20 annular carbon atoms (i.e., C₆₋₂₀aryl), 6 to 16 annular carbon atoms (i.e., C₆₋₁₆aryl), 6 to 12 annular carbon atoms (i.e., C₆₋₁₂aryl), or 6 to 10 annular carbon atoms (i.e., C₆₋₁₀aryl). Examples of aryl moieties include, but are not limited to, phenyl, naphthyl, fluorenyl, and anthryl.

The term “cycloalkyl”, as used herein, refers to a saturated or partially unsaturated carbocyclic ring moiety. The term “cycloalkyl” encompasses monocyclic and polycyclic ring moieties, wherein the polycyclic moieties may be fused, branched, or spiro. Cycloalkyl includes cycloalkenyl groups, wherein the ring moiety comprises at least one annular double bond. Cycloalkyl includes any polycyclic carbocyclic ring moiety comprising at least one non-aromatic ring, regardless of the point of attachment to the remainder of the molecule. As used herein, cycloalkyl includes rings comprising, for example, 3 to 20 annular carbon atoms (i.e., a C₃₋₂₀cycloalkyl), 3 to 16 annular carbon atoms (i.e., a C₃₋₁₆cycloalkyl), 3 to 12 annular carbon atoms (i.e., a C₃₋₁₂cycloalkyl), 3 to 10 annular carbon atoms (i.e., a C₃₋₁₀cycloalkyl), 3 to 8 annular carbon atoms (i.e., a C₃₋₈cycloalkyl), 3 to 6 annular carbon atoms (i.e., a C₃₋₆cycloalkyl), or 3 to 5 annular carbon atoms (i.e., a C₃₋₅cycloalkyl). Monocyclic cycloalkyl ring moieties include, for example, cyclopropyl, cyclobutyl, cyclopentyl, cyclohexyl, cycloheptyl, and cyclooctyl. Polycyclic groups include, for example, bicyclo[2.2.1]heptanyl, bicyclo[2.2.2]octanyl, adamantyl, norbomyl, decalinyl, 7,7-dimethyl-bicyclo [2.2.1]heptanyl, and the like. Still further, cycloalkyl also includes spiro cycloalkyl ring moieties, for example, spiro[2.5]octanyl, spiro[4.5]decanyl, or spiro [5.5]undecanyl.

The term “halo”, as used herein, refers to atoms occupying groups VIIA of The Periodic Table and includes fluorine (fluoro), chlorine (chloro), bromine (bromo), and iodine (iodo).

The term “heteroaryl”, as used herein, refers to an aromatic (fully unsaturated) ring moiety that comprises one or more annular heteroatoms independently selected from the group consisting of nitrogen, oxygen, and sulfur. The term “heteroaryl” includes both monocyclic and polycyclic fused-ring moieties. As used herein, a heteroaryl comprises, for example, 5 to 20 annular atoms (i.e., a 5-20 membered heteroaryl), 5 to 16 annular atoms (i.e., a 5-16 membered heteroaryl), 5 to 12 annular atoms (i.e., a 5-12 membered heteroaryl), 5 to 10 annular atoms (i.e., a 5-10 membered heteroaryl), 5 to 8 annular atoms (i.e., a 5-8 membered heteroaryl), or 5 to 6 annular atoms (i.e., a 5-6 membered heteroaryl). Any monocyclic or polycyclic aromatic ring moiety comprising one or more annular heteroatoms is considered a heteroaryl, regardless of the point of attachment to the remainder of the molecule (i.e., the heteroaryl moiety may be attached to the remainder of the molecule through any annular carbon or any annular heteroatom of the heteroaryl moiety). Examples of heteroaryl groups include, but are not limited to, acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, triazolyl, tetrazolyl, and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl, and imidazo[1,5-a]pyridinyl, wherein the heteroaryl can be bound via either ring of the fused system.

The term “heterocyclyl”, as used herein, refers to a saturated or partially unsaturated cyclic moiety that encompasses one or more annular heteroatoms independently selected from the group consisting of nitrogen, oxygen, and sulfur. The term “heterocyclyl” includes both monocyclic and polycyclic ring moieties, wherein the polycyclic ring moieties may be fused, bridged, or spiro. Any non-aromatic monocyclic or polycyclic ring moiety comprising at least one annular heteroatom is considered a heterocyclyl, regardless of the point of attachment to the remainder of the molecule (i.e., the heterocyclyl moiety may be attached to the remainder of the molecule through any annular carbon or any annular heteroatom of the heterocyclyl moiety). Further, the term heterocyclyl is intended to encompass any polycyclic ring moiety comprising at least one annular heteroatom wherein the polycyclic ring moiety comprises at least one non-aromatic ring, regardless of the point of attachment to the remainder of the molecule. As used herein, a heterocyclyl comprises, for example, 3 to 20 annular atoms (i.e., a 3-20 membered heterocyclyl), 3 to 16 annular atoms (i.e., a 3-16 membered heterocyclyl), 3 to 12 annular atoms (i.e., a 3-12 membered heterocyclyl), 3 to 10 annular atoms (i.e., a 3-10 membered heterocyclyl), 3 to 8 annular atoms (i.e., a 3-8 membered heterocyclyl), 3 to 6 annular atoms (i.e., a 3-6 membered heterocyclyl), 3 to 5 annular atoms (i.e., a 3-5 membered heterocyclyl), 5 to 8 annular atoms (i.e., a 5-8 membered heterocyclyl), or 5 to 6 annular atoms (i.e., a 5-6 membered heterocyclyl). Examples of heterocyclyl groups include, e.g., azetidinyl, azepinyl, benzodioxolyl, benzo[b]1[1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiophenyl (i.e., thienyl), thiomorpholinyl, thiamorpholinyl, 1-oxo-thiomorpholinyl, and 1,1-dioxo-thiomorpholinyl. Examples of spiro heterocyclyl rings include, but are not limited to, bicyclic and tricyclic ring systems, such as oxabicyclo[2.2.2]octanyl, 2-oxa-7-azaspiro[3.5]nonanyl, 2-oxa-6-azaspiro[3.4]octanyl, and 6-oxa-1-azaspiro[3.3]heptanyl. Examples of fused heterocyclyl rings include, but are not limited to, 1,2,3,4-tetrahydroisoquinolinyl, 4,5,6,7-tetrahydrothieno[2,3-c]pyridinyl, indolinyl, and isoindolinyl, where the heterocyclyl can be bound via either ring of the fused system.

The terms “optional” and “optionally”, as used herein, mean that the subsequently described event or circumstance may or may not occur and that the description includes instances where the event or circumstance occurs and instances where it does not. Accordingly, the term “optionally substituted” infers that any one or more (e.g., 1, 2, 1 to 5, 1 to 3, 1 to 2, etc.) hydrogen atoms on the designated atom or moiety or group may be replaced or not replaced by an atom or moiety or group other than hydrogen. By way of illustration and not limitation, the phrase “methyl optionally substituted with one or more chloro” encompasses —CH₃, —CH₂Cl, —CHCl₂, and —CCl₃ moieties.

It is understood that aspects and embodiments described herein as “comprising” include “consisting of” and “consisting essentially of” embodiments.

The term “pharmaceutically acceptable salt”, as used herein, of a given compound refers to salts that retain the biological effectiveness and properties of the given compound and which are not biologically or otherwise undesirable. “Pharmaceutically acceptable salts” include, for example, salts with inorganic acids, and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. See, e.g., Handbook of Pharmaceutical Salts Properties, Selection, and Use, International Union of Pure and Applied Chemistry, John Wiley & Sons (2008), which is incorporated herein by reference. Those skilled in the art will recognize various synthetic methodologies that may be used to prepare nontoxic pharmaceutically acceptable addition salts. Pharmaceutically acceptable acid addition salts may be prepared from inorganic or organic acids. Salts derived from inorganic acids include, e.g., hydrochloric acid, hydrobromic acid, sulfuric acid, nitric acid, phosphoric acid, and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, trifluoroacetic acid, and the like. Likewise, pharmaceutically acceptable base addition salts can be prepared from inorganic or organic bases. Salts derived from inorganic bases include, by way of example only, sodium, potassium, lithium, aluminum, ammonium, calcium, and magnesium salts. Salts derived from organic bases include, but are not limited to, salts of primary, secondary, and tertiary amines. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

Isotopically labeled forms of the compounds depicted herein may be prepared. Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. In some embodiments, a compound of formula (A), or formula (A′) is provided wherein one or more hydrogen is replaced by deuterium or tritium.

Some of the compounds provided herein may exist as tautomers. Tautomers are in equilibrium with one another. By way of illustration, amide containing compounds may exist in equilibrium with imidic acid tautomers. Regardless of which tautomer is shown and regardless of the nature of the equilibrium among tautomers, the compounds of this disclosure are understood by one of ordinary skill in the art to comprise both amide and imidic acid tautomers. Thus, for example, amide-containing compounds are understood to include their imidic acid tautomers. Likewise, imidic-acid containing compounds are understood to include their amide tautomers.

Also provided herein are prodrugs of the compounds depicted herein, or a pharmaceutically acceptable salt thereof. Prodrugs are compounds that may be administered to an individual and release, in vivo, a compound depicted herein as the parent drug compound. It is understood that prodrugs may be prepared by modifying a functional group on a parent drug compound in such a way that the modification is cleaved in vivo to release the parent drug compound. See, e.g., Rautio, J., Kumpulainen, H., Heimbach, T. et al. Prodrugs: design and clinical applications. Nat Rev Drug Discov 7, 255-270 (2008), which is incorporated herein by reference.

The compounds of the present disclosure, or their pharmaceutically acceptable salts, may include an asymmetric center and may thus give rise to enantiomers, diastereomers, and other stereoisomeric forms that may be defined, in terms of absolute stereochemistry, as (R)- or (S)- (or as (D)- or (L)- for amino acids). The present disclosure is meant to include all such possible isomers, as well as their racemic and optically pure forms and mixtures thereof in any ratio. Optically active (+) and (−), (R)- and (S)-, or (D)- and (L)-isomers may be prepared using chiral synthons or chiral reagents, or may be resolved using conventional techniques, for example, chromatography and/or fractional crystallization. Conventional techniques for the preparation/isolation of individual enantiomers include chiral synthesis from a suitable optically pure precursor or the resolution of the racemate (or the racemate of a salt or derivative) using, for example, chiral high pressure liquid chromatography (HPLC), and chiral SFC (supercritical fluid chromatography). When the compounds described herein contain olefinic double bonds or other centers of geometric asymmetry, unless specified otherwise, it is intended that the present disclosure includes both E and Z geometric isomers. Likewise, cis- and trans- are used in their conventional sense to describe relative spatial relationships.

A “stereoisomer” refers to a compound made up of the same atoms bonded by the same bonds, but having different three-dimensional structures, which are not interchangeable. The present disclosure contemplates various stereoisomers, or mixtures thereof, and includes “enantiomers,” which refers to two stereoisomers whose structures are non-superimposable mirror images of one another. “Diastereomers” are stereoisomers that have at least two asymmetric atoms, but which are not mirror images of each other.

Where enantiomeric and/or diastereomeric forms exist of a given structure, flat bonds indicate that all stereoisomeric forms of the depicted structure may be present, e.g.,

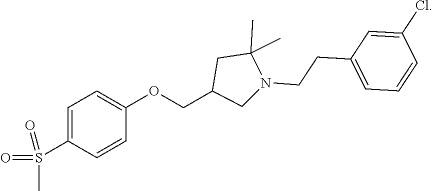 [SMILES: CC1(C)CC(COc2ccc(S(C)(=O)=O)cc2)CN1CCc1cccc(Cl)c1]

Where enantiomeric forms exist of a given structure, flat bonds and the presence of a “*” symbol indicate that the composition is made up of at least 90%, by weight, of a single isomer with unknown stereochemistry, e.g.,

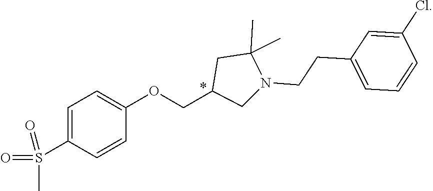 [SMILES: CC1(C)CC(COc2ccc(S(C)(=O)=O)cc2)CN1CCc1cccc(Cl)c1]

Where enantiomeric and/or diastereomeric forms exist of a given structure, wedged or hashed bonds indicate the composition is made up of at least 90%, by weight, of a single enantiomer or diastereomer with known stereochemistry, e.g.,

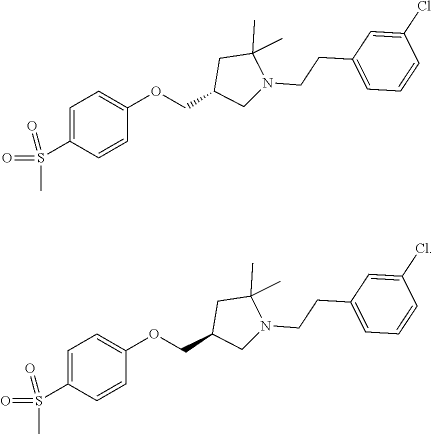 [SMILES: CC1(C)C[C@@H](COc2ccc(S(C)(=O)=O)cc2)CN1CCc1cccc(Cl)c1 ; CC1(C)C[C@H](COc2ccc(S(C)(=O)=O)cc2)CN1CCc1cccc(Cl)c1]

Where enantiomeric and/or diastereomeric forms exist of a given structure with two stereocenters, flat bonds and the presence of two “& 1” symbols indicate the composition is made up of a pair of enantiomers with unknown relative stereochemistry, e.g.,

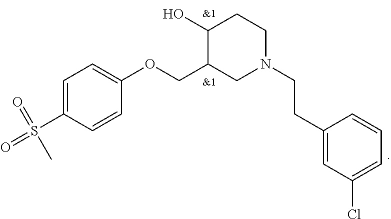 [SMILES: CS(=O)(=O)c1ccc(OCC2CN(CCc3cccc(Cl)c3)CCC2O)cc1]

Where enantiomeric and/or diastereomeric forms exist of a given structure with two stereocenters, wedged and/or dashed bonds and the presence of two “&l” symbols indicate the composition is made up of a pair of enantiomers with known relative stereochemistry, e.g.,

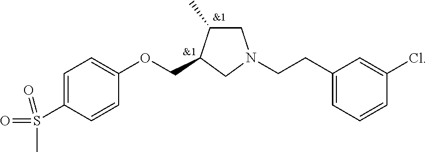 [SMILES: C[C@@H]1CN(CCc2cccc(Cl)c2)C[C@H]1COc1ccc(S(C)(=O)=O)cc1]

Where enantiomeric and/or diastereomeric forms exist of a given structure with two stereocenters, wedged and/or dashed bonds and the presence of two “or1” symbols indicate the composition is made up of at least 90%, by weight, a single stereoisomer with known relative stereochemistry but unknown absolute stereochemistry, e.g.,

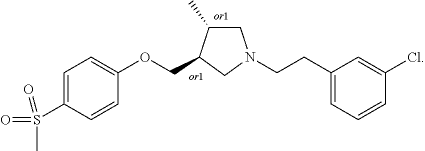 [SMILES: C[C@@H]1CN(CCc2cccc(Cl)c2)C[C@H]1COc1ccc(S(C)(=O)=O)cc1]

Where enantiomeric and/or diastereomeric forms exist of a given structure with two stereocenters, flat bonds and the presence of two “*” symbols indicate the composition is made up of at least 90%, by weight, of a single enantiomer or diastereomer with unknown stereochemistry, e.g.,

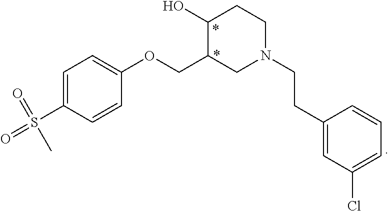 [SMILES: CS(=O)(=O)c1ccc(OCC2CN(CCc3cccc(Cl)c3)CCC2O)cc1]

Abbreviations used are those conventional in the art and are in accordance with the Periodic Table of the Elements, CAS version, Handbook of Chemistry and Physics, 75^{th} Ed. The following examples are intended to be illustrative only and not limiting in any way.

| | | | |
|---|---|---|---|
| ° C. | degrees Celsius | LiHMDS | lithium bis(trimethylsilyl)amide |
| μL | microliter | mCPBA or | meta-chloroperoxybenzoic acid |
| | | m-CPBA | |
| [M + XX]⁺ | observed mass | MeOH | methanol |
| AC₅₀ | half-maximal activity | MeCN | acetonitrile |
| | concentration | | |
| AcOH | acetic acid | m | multiplet (NMR) |
| AIBN | azobisisobutyronitrile | mg | milligrams |
| app | apparent (NMR) | min | minutes |
| b | broad (as in “br s” to | mL | milliliter |
| | indicate a broad singlet) | | |
| BH₃•THF | borane-tetrahydrofuran | mmol | millimole |
| | complex | | |
| BBr₃ | boron tribromide | mM | millimolar |
| Calc'd | calculated | M | molarity or molar |
| Cbz—Cl or | benzyl chloroformate | MS | mass spectrometry |
| CbzCl | | | |
| CO₂ | carbon dioxide | MsCl | methanesulfonyl chloride |
| Cs₂CO₃ | cesium carbonate | MTBE | methyl tert-butyl ether |
| d | deuterated (NMR solvents) | n/a | not applicable |
| d | doublet (NMR) | NH₄ | ammonium |
| dd | doublet of doublets (NMR) | NH₄OH | ammonium hydroxide |
| DCE | 1,2-dichloroethane | NH₄HCO₃ | ammonium bicarbonate |
| DCM | dichloromethane | Na₂SO₄ | sodium sulfate |
| DIAD | diisopropyl | NaBH₃CN | sodium cyanoborohydride |
| | azodicarboxylate | | |
| DMF | N,N-dimethylformamide | NMR | nuclear magnetic resonance |
| DMP | Dess-Martin periodinane | NaOH | sodium hydroxide |
| EC₅₀ | half-maximal effective | Pd(dppf)Cl₂ | [1,1′- |
| | concentration | | bis(diphenylphosphino)ferro- |
| | | | cene]dichloropalladium(II) |
| EtOAc | ethyl acetate | Pd/C | palladium on carbon |
| EtOH | ethanol | pH | potential of hydrogen |
| g | grams | PPh₃ | triphenyl phosphine |
| h | hours | s | singlet (NMR) |
| H | hydrogen | SFC | supercritical fluid |
| | | | chromatography |
| HCl | hydrochloric acid | t | triplet (NMR) |
| HPLC | high-performance liquid | TBAB | tetrabutylammonium bromide |
| | chromatography | | |
| In vacuo | in a vacuum | TEA | triethylamine |
| IUPAC | International Union of Pure | TFA | trifluoroacetic acid |
| | and Applied Chemistry | | |
| MHz | megahertz | THF | tetrahydrofuran |
| J | J-coupling value (NMR) | TMAD | tetramethylazodicarboxamide |
| K₂CO₃ | potassium carbonate | TMSCl | trimethylsilyl chloride |

### Compounds

Provided herein is a compound of formula (A′):

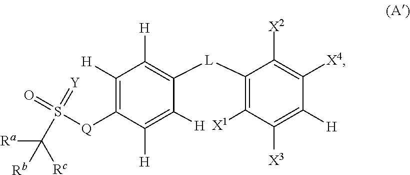

or a stereoisomer or tautomer thereof, or a pharmaceutically acceptable salt of any of the foregoing, wherein:
Q is absent or is —N—(C₁₋₆alkyl);
Y is O or —N—(C₁₋₆alkyl),
   provided that, when Q is —N(C₁₋₆alkyl), then Y is O;
R^{a}, R^{b}, and R^{c} are each independently H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl,
or any two of R^{a}, R^{b}, and R^{c} are taken, together with the atoms to which they are attached, to form a C₃₋₆cycloalkyl or a 3-6 membered heterocyclyl, and the other of R^{a}, R^{b}, and R^{c} is H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl;
L is selected from the group consisting of:

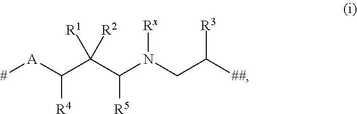

wherein
   A is O, NH, N(C₁₋₆alkyl), CH₂, or CH(C₁₋₆alkyl);
   R^{x} is H,
   or R^{x} is taken together with one of R¹ and R², and the atoms to which they are attached, to form a 3-8 membered heterocyclyl, wherein the 3-8 membered heterocyclyl is substituted with n independently selected R⁹ substituents, wherein n is an integer from 0-6, and R^{g} is —OH, halo, C₁₋₆alkyl, or C₁₋₆alkoxy;
   R¹ and R² are independently H, halo, or —OH,
   or one of R¹ and R² is taken together with R^{x}, and the atoms to which they are attached, to form a 3-8 membered heterocyclyl, wherein the 3-8 membered heterocyclyl is substituted with n independently selected R^{g} substituents, wherein n is an integer from 0-6, and R^{g} is —OH, halo, C₁₋₆alkyl, or C₁₋₆alkoxy, and the other of R¹ and R² is H, halo, or —OH;
   R³ is H, —OH, halo, or C₁₋₆alkoxy; and
   R⁴ and R⁵ are independently H,
   or R⁴ and R⁵ are taken, together with the atoms to which they are attached, to form a C₃₋₈cycloalkyl,
   provided that either:
      (1) R^{x} is taken together with one of R¹ and R², and the atoms to which they are attached, to form a 3-8 membered heterocyclyl, wherein the 3-8 membered heterocyclyl is optionally substituted with one or more —OH, C₁₋₆alkyl, or C₁₋₆alkoxy, or
      (2) R⁴ and R⁵ are taken, together with the atoms to which they are attached, to form a C₃₋₈cycloalkyl,

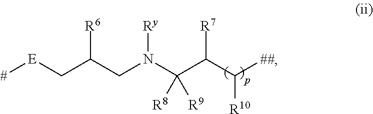

wherein
E is O, NH, N(C₁₋₆alkyl), CH₂, or CH(C₁₋₆alkyl);
p is 0 or 1,
   provided that, when p is 1, then E is O;
R⁶ is H or —OH;
R^{y} is H,
or R^{y} is taken together with R⁷, and the atoms to which they are attached, to form a 3-8 membered heterocyclyl,
or R^{y} is taken together with one of R⁸ and R⁹, and the atoms to which they are attached, to form a 3-8 membered heterocyclyl;
R⁷ is H,
or R⁷ is taken together with R^{y}, and the atoms to which they are attached, to form a 3-8 membered heterocyclyl;
R⁸ and R⁹ are independently H or C₁₋₆alkyl,
or one of R⁸ and R⁹ is taken together with R^{y}, and the atoms to which they are attached, to form a 3-8 membered heterocyclyl, and the other of R⁸ and R⁹ is H or C₁₋₆alkyl,
or one of R⁸ and R⁹ is taken together with R¹⁰, and the atoms to which they are attached, to form a C₃₋₈cycloalkyl, and the other of R⁸ and R⁹ is H or C₁₋₆alkyl; and
R¹⁰ is H,
or R¹⁰ is taken together with one of R⁸ and R⁹, and the atoms to which they are attached, to form a C₃₋₈cycloalkyl,
provided that:
   (1) R^{y} is taken together with R⁷, and the atoms to which they are attached, to form a 3-8 membered heterocyclyl, or
   (2) R^{y} is taken together with one of R⁸ and R⁹, and the atoms to which they are attached, to form a 3-8 membered heterocyclyl, or
   (3) one of R⁸ and R⁹ is taken together with R¹⁰ and the atoms to which they are attached, to form a C₃₋₈cycloalkyl, and

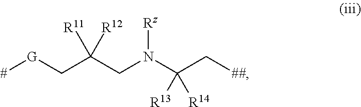

wherein
G is O, NH, N(C₁₋₆alkyl), CH₂, or CH(C₁₋₆alkyl);
R^{z} is H or C₁₋₆alkyl, wherein the C₁₋₆alkyl is optionally substituted with one or more C₃₋₈cycloalkyl;
R¹¹ and R¹² are independently H, —OH, halo, or C₁₋₆alkyl; and
R¹³ and R¹⁴ are independently H, C₁₋₆alkyl, or C₃₋₈cycloalkyl,
or R¹³ and R¹⁴ are taken, together with the atoms to which they are attached, to form a 3-8 membered heterocyclyl,
wherein, for each of (i)-(iii), #denotes the point of attachment to the phenyl ring bearing moiety Q, and ##denotes the point of attachment to the phenyl ring bearing moieties X¹-X⁴; and
X¹, X², X³, and X⁴ are, independently of each other, H, halo, —CN, C₁₋₆alkyl, C₁₋₆alkoxy, or SF₅ wherein the C₁₋₆alkyl or C₁₋₆alkoxy is optionally substituted with one or more halo,
   provided that at least one of X¹, X², X³, and X⁴ is halo, —CN, C₁₋₆alkyl, C₁₋₆alkoxy, or SF₅, wherein the C₁₋₆alkyl or C₁₋₆alkoxy is optionally substituted with one or more halo.

Provided herein is a compound of formula (A):

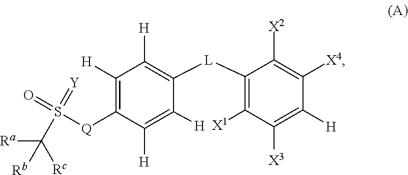

or a stereoisomer or tautomer thereof, or a pharmaceutically acceptable salt of any of the foregoing, wherein:
Q is absent or is —N—(C₁₋₆alkyl);
Y is O or —N—(C₁₋₆alkyl),
   provided that, when Q is —N(C₁₋₆alkyl), then Y is O;
R^{a}, R^{b}, and R^{c} are each independently H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl, or any two of R^{a}, R^{b}, and R^{c} are taken, together with the atoms to which they are attached, to form a C₃₋₆cycloalkyl or a 3-6 membered heterocyclyl, and the other of R^{a}, R^{b}, and R^{c} is H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl;
L is selected from the group consisting of:

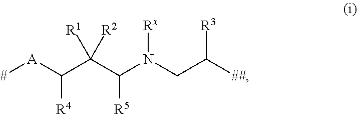

wherein
   A is O, NH, N(C₁₋₆alkyl), CH₂, or CH(C₁₋₆alkyl);
   R^{x} is H,
   or R^{x} is taken together with one of R¹ and R², and the atoms to which they are attached, to form a 3-8 membered heterocyclyl, wherein the 3-8 membered heterocyclyl is substituted with n independently selected R^{g} substituents, wherein n is an integer from 0-6, and R^{g} is —OH, halo, C₁₋₆alkyl, or C₁₋₆alkoxy;
   R¹ and R² are independently H, halo, or —OH,
   or one of R¹ and R² is taken together with R^{x}, and the atoms to which they are attached, to form a 3-8 membered heterocyclyl, wherein the 3-8 membered heterocyclyl is substituted with n independently selected R^{g} substituents, wherein n is an integer from 0-6, and R^{g} is —OH, halo, C₁₋₆alkyl, or C₁₋₆alkoxy, and the other of R¹ and R² is H, halo, or —OH;
   R³ is H, —OH, halo, or C₁₋₆alkoxy; and
   R⁴ and R⁵ are independently H,
   or R⁴ and R⁵ are taken, together with the atoms to which they are attached, to form a C₃₋₈cycloalkyl,
   provided that either:
      (1) R^{x} is taken together with one of R¹ and R², and the atoms to which they are attached, to form a 3-8 membered heterocyclyl, wherein the 3-8 membered heterocyclyl is optionally substituted with one or more —OH, C₁₋₆alkyl, or C₁₋₆alkoxy, or
      (2) R⁴ and R⁵ are taken, together with the atoms to which they are attached, to form a C₃₋₈cycloalkyl,

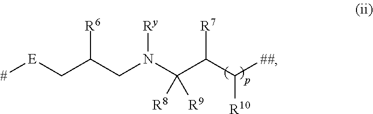

wherein
E is O, NH, N(C₁₋₆alkyl), CH₂, or CH(C₁₋₆alkyl);
p is 0 or 1,
   provided that, when p is 1, then E is O;
R⁶ is H or —OH;
R^{y} is H,
or R^{y} is taken together with R⁷, and the atoms to which they are attached, to form a 3-8 membered heterocyclyl,
or R^{y} is taken together with one of R⁸ and R⁹, and the atoms to which they are attached, to form a 3-8 membered heterocyclyl;
R⁷ is H,
or R⁷ is taken together with R^{y}, and the atoms to which they are attached, to form a 3-8 membered heterocyclyl;
R⁸ and R⁹ are independently H or C₁₋₆alkyl,
or one of R⁸ and R⁹ is taken together with R^{y}, and the atoms to which they are attached, to form a 3-8 membered heterocyclyl, and the other of R⁸ and R⁹ is H or C₁₋₆alkyl,
or one of R⁸ and R⁹ is taken together with R¹⁰, and the atoms to which they are attached, to form a C₃₋₈cycloalkyl, and the other of R⁸ and R⁹ is H or C₁₋₆alkyl; and
R¹⁰ is H,
or R¹⁰ is taken together with one of R⁸ and R⁹, and the atoms to which they are attached, to form a C₃₋₈cycloalkyl,
provided that:
   (1) R^{y} is taken together with R⁷, and the atoms to which they are attached, to form a 3-8 membered heterocyclyl, or
   (2) R^{y} is taken together with one of R⁸ and R⁹, and the atoms to which they are attached, to form a 3-8 membered heterocyclyl, or
   (3) one of R⁸ and R⁹ is taken together with R¹⁰ and the atoms to which they are attached, to form a C₃₋₈cycloalkyl, and

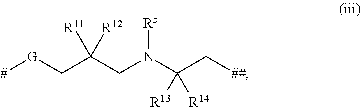

wherein
G is O, NH, N(C₁₋₆alkyl), CH₂, or CH(C₁₋₆alkyl);
R^{z} is H or C₁₋₆alkyl, wherein the C₁₋₆alkyl is optionally substituted with one or more C₃₋₈cycloalkyl;
R¹¹ and R¹² are independently H, —OH, halo, or C₁₋₆alkyl; and
R¹³ and R¹⁴ are independently H, C₁₋₆alkyl, or C₃₋₈cycloalkyl,
or R¹³ and R¹⁴ are taken, together with the atoms to which they are attached, to form a 3-8 membered heterocyclyl,
wherein, for each of (i)-(iii), #denotes the point of attachment to the phenyl ring bearing moiety Q, and ##denotes the point of attachment to the phenyl ring bearing moieties X¹-X⁴; and
X¹, X², X³, and X⁴ are, independently of each other, H, halo, —CN, C₁₋₆alkyl, or C₁₋₆alkoxy, wherein the C₁₋₆alkyl or C₁₋₆alkoxy is optionally substituted with one or more halo,
   provided that at least one of X¹, X², X³, and X⁴ is halo, —CN, C₁₋₆alkyl, or C₁₋₆alkoxy, wherein the C₁₋₆alkyl or C₁₋₆alkoxy is optionally substituted with one or more halo.

Any embodiments provided herein of a compound of formula (A), or a stereoisomer or tautomer thereof, or a pharmaceutically acceptable salt of any of the foregoing, or any variation or embodiment thereof, are also embodiments of a compound of formula (A′), or a stereoisomer or tautomer thereof, or a pharmaceutically acceptable salt of any of the foregoing, or any variation or embodiment thereof.

In some embodiments, provided is a compound of formula (A), or a stereoisomer or tautomer thereof, or a pharmaceutically acceptable salt of any of the foregoing, wherein L is

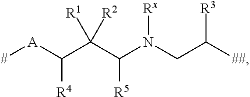

such that the compound of formula (A) is a compound of formula (I):

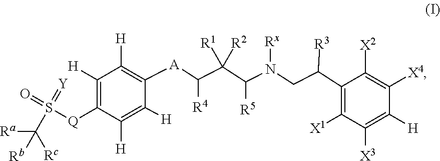

or a stereoisomer or tautomer thereof, or a pharmaceutically acceptable salt of any of the foregoing. In some variations, the X¹, X², X³, X⁴, R¹, R², R³, R⁴, R⁵, R^{a}, R^{b}, R^{c}, R^{x}, A Q, and Y of formula (I) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (I), or a stereoisomer or tautomer thereof, or a pharmaceutically acceptable salt of any of the foregoing, wherein A is O, such that the compound is of formula (I-A1):

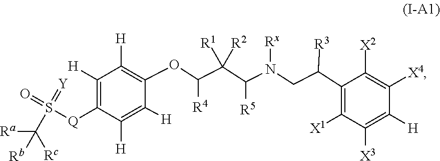

or a stereoisomer or tautomer thereof, or a pharmaceutically acceptable salt of any of the foregoing. In some variations, the X¹, X², X³, X⁴, R¹, R², R³, R⁴, R⁵, R^{a}, R^{b}, R^{c}, R^{x}, Q, and Y of formula (I-A1) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (I), or a stereoisomer or tautomer thereof, or a pharmaceutically acceptable salt of any of the foregoing, wherein A is NH, such that the compound is of formula (I-A2):

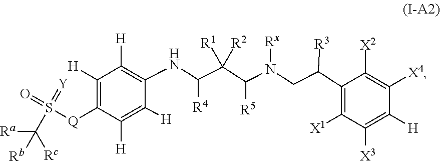

or a stereoisomer or tautomer thereof, or a pharmaceutically acceptable salt of any of the foregoing. In some embodiments, A is N(C₁₋₆alkyl). In some embodiments, A is N(CH₃). In some variations, X¹, X², X³, X⁴, R¹, R², R³, R⁴, R⁵, R^{a}, R^{b}, R^{c}, R^{x}, Q, and Y of formula (I-A2) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (I), or a stereoisomer or tautomer thereof, or a pharmaceutically acceptable salt of any of the foregoing, wherein A is CH₂, such that the compound is of formula (I-A3):

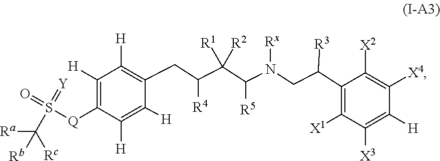

or a stereoisomer or tautomer thereof, or a pharmaceutically acceptable salt of any of the foregoing. In some embodiments, A is CH(C₁₋₆alkyl). In some embodiments, A is CH(CH₃). In some variations, X¹, X², X³, X⁴, R¹, R², R³, R⁴, R⁵, R^{a}, R^{b}, R^{c}, R^{x}, Q, and Y of formula (I-A3) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (I), such as a compound of formula (I-A1), (I-A2), or (I-A3), or a pharmaceutically acceptable salt of any of the foregoing, wherein R^{x} is taken together with one of R¹ and R², and the atoms to which they are attached, to form a 3-8 membered heterocyclyl, wherein the 3-8 membered heterocyclyl is substituted with n independently selected R^{g} substituents, wherein n is an integer from 0-6, and R^{g} is —OH, halo, C₁₋₆alkyl, or C₁₋₆alkoxy. In some embodiments, R^{x} is taken together with one of R¹ and R², and the atoms to which they are attached, to form a 3-6 membered heterocyclyl, wherein the 3-6 membered heterocyclyl is substituted with n independently selected R^{g} substituents, wherein n is an integer from 0-6, and R^{g} is —OH, halo, C₁₋₆alkyl, or C₁₋₆alkoxy. In some embodiments, R^{x} is taken together with one of R¹ and R², and the atoms to which they are attached, to form a 5-8 membered heterocyclyl, wherein the 5-8 membered heterocyclyl is substituted with n independently selected R^{g} substituents, wherein n is an integer from 0-6, and R^{g} is —OH, halo, C₁₋₆alkyl, or C₁₋₆alkoxy. In some embodiments, R^{x} is taken together with one of R¹ and R², and the atoms to which they are attached, to form a 5-6 membered heterocyclyl, wherein the 5-6 membered heterocyclyl is substituted with n independently selected R^{g} substituents, wherein n is an integer from 0-6, and R^{g} is —OH, halo, C₁₋₆alkyl, or C₁₋₆alkoxy. R^{x} is taken together with one of R¹ and R², and the atoms to which they are attached, to form a 5-membered heterocyclyl, wherein the 5-membered heterocyclyl is substituted with n independently selected R^{g} substituents, wherein n is an integer from 0-6, and R^{g} is —OH, halo, C₁₋₆alkyl, or C₁₋₆alkoxy. R^{x} is taken together with one of R¹ and R², and the atoms to which they are attached, to form a 6-membered heterocyclyl, wherein the 6-membered heterocyclyl is substituted with n independently selected R^{g} substituents, wherein n is an integer from 0-6, and R^{g} is —OH, halo, C₁₋₆alkyl, or C₁₋₆alkoxy. In some variations, the embodiments provided herein also apply to a compound of formula (A′) or a stereoisomer or tautomer thereof, or a pharmaceutically acceptable salt of any of the foregoing, or any variation or embodiment thereof.

In some embodiments, provided herein is a compound of formula (A) or formula (I), such as a compound of formula (I-A1), or a pharmaceutically acceptable salt of any of the foregoing, wherein R^{x} is taken together with one of R¹ and R², and the atoms to which they are attached, to form a 5-membered heterocyclyl, wherein the 5-membered heterocyclyl is substituted with n independently selected R^{g} substituents, wherein n is an integer from 0-6, and R^{g} is —OH, halo, C₁₋₆alkyl, or C₁₋₆alkoxy, wherein the compound is of formula (I-B1):

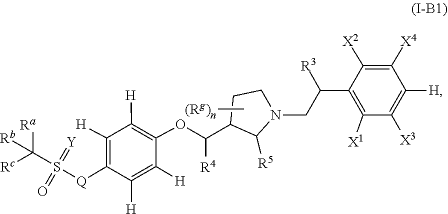

or a stereoisomer or tautomer thereof, or a pharmaceutically acceptable salt of any of the foregoing. In some variations, X¹, X², X³, X⁴, R³, R⁴, R⁵, R^{a}, R^{b}, R^{c}, R⁹, R^{x}, Q, Y and n of formula (I-B1) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (I), such as a compound of formula (I-A2), or a pharmaceutically acceptable salt of any of the foregoing, wherein R^{x} is taken together with one of R¹ and R², and the atoms to which they are attached, to form a 5-membered heterocyclyl, wherein the 5-membered heterocyclyl is substituted with n independently selected R^{g} substituents, wherein n is an integer from 0-6, and R^{g} is —OH, halo, C₁₋₆alkyl, or C₁₋₆alkoxy, wherein the compound is of formula (I-B2):

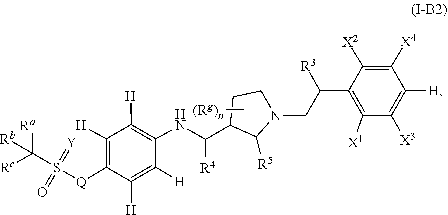

or a stereoisomer or tautomer thereof, or a pharmaceutically acceptable salt of any of the foregoing. In some variations, X¹, X², X³, X⁴, R³, R⁴, R⁵, R^{a}, R^{b}, R^{c}, R^{g}, R^{x}, Q, Y and n of formula (I-B2) are as defined of formula (A′), or a stereoisomer or tautomer thereof, or a pharmaceutically acceptable salt of any of the foregoing, or any variation or embodiment thereof.

In some embodiments, provided herein is a compound of formula (A) or formula (I), such as a compound of formula (I-A3), or a pharmaceutically acceptable salt of any of the foregoing, wherein R^{x} is taken together with one of R¹ and R², and the atoms to which they are attached, to form a 5-membered heterocyclyl, wherein the 5-membered heterocyclyl is substituted with n independently selected R^{g} substituents, wherein n is an integer from 0-6, and R^{g} is —OH, halo, C₁₋₆alkyl, or C₁₋₆alkoxy, wherein the compound is of formula (I-B3):

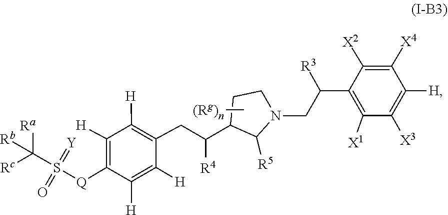

or a stereoisomer or tautomer thereof, or a pharmaceutically acceptable salt of any of the foregoing. In some variations, X¹, X², X³, X⁴, R³, R⁴, R⁵, R^{a}, R^{b}, R^{c}, R^{g}, R^{x}, Q, Y and n of formula (I-B3) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (I), such as a compound of formula (I-A1), or a pharmaceutically acceptable salt of any of the foregoing, wherein R^{x} is taken together with one of R¹ and R², and the atoms to which they are attached, to form a 6-membered heterocyclyl, wherein the 6-membered heterocyclyl is substituted with n independently selected R^{g} substituents, wherein n is an integer from 0-6, and R^{g} is —OH, halo, C₁₋₆alkyl, or C₁₋₆alkoxy, wherein the compound is of formula (I-C1):

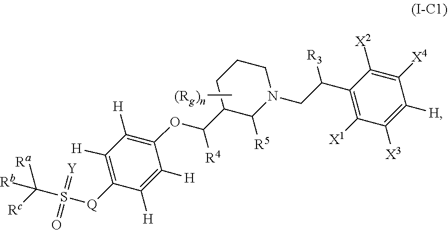

or a stereoisomer or tautomer thereof, or a pharmaceutically acceptable salt of any of the foregoing. In some variations, X¹, X², X³, X⁴, R³, R⁴, R⁵, R^{a}, R^{b}, R^{c}, R^{g}, R^{x}, Q, Y and n of formula (I-C1) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (I), such as a compound of formula (I-A2), or a pharmaceutically acceptable salt of any of the foregoing, wherein R^{x} is taken together with one of R¹ and R², and the atoms to which they are attached, to form a 6-membered heterocyclyl, wherein the 6-membered heterocyclyl is substituted with n independently selected R^{g} substituents, wherein n is an integer from 0-6, and R^{g} is —OH, halo, C₁₋₆alkyl, or C₁₋₆alkoxy, wherein the compound is of formula (I-C2):

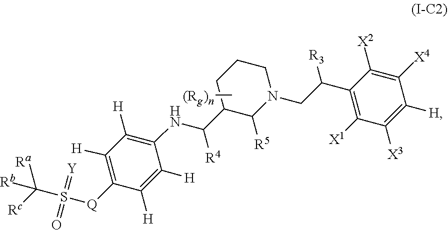

or a stereoisomer or tautomer thereof, or a pharmaceutically acceptable salt of any of the foregoing. In some variations, X¹, X², X³, X⁴, R³, R⁴, R⁵, R^{a}, R^{b}, R^{c}, R^{g}, R^{x}, Q, Y and n of formula (I-C2) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (I), such as a compound of formula (I-A3), or a pharmaceutically acceptable salt of any of the foregoing, wherein R^{x} is taken together with one of R¹ and R², and the atoms to which they are attached, to form a 6-membered heterocyclyl, wherein the 6-membered heterocyclyl is substituted with n independently selected R^{g} substituents, wherein n is an integer from 0-6, and R^{g} is —OH, halo, C₁₋₆alkyl, or C₁₋₆alkoxy, wherein the compound is of formula (I-C3):

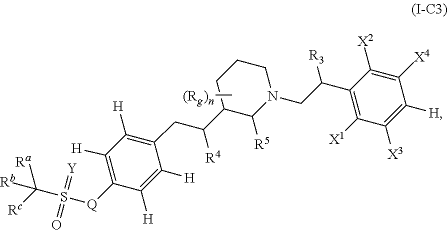

or a stereoisomer or tautomer thereof, or a pharmaceutically acceptable salt of any of the foregoing. In some variations, X¹, X², X³, X⁴, R³, R⁴, R⁵, R^{a}, R^{b}, R^{c}, R^{g}, R^{x}, Q, Y and n of formula (I-C3) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (I), such as a compound of formula (I-A1), or a pharmaceutically acceptable salt of any of the foregoing, wherein R^{x} is taken together with one of R¹ and R², and the atoms to which they are attached, to form a 6-membered heterocyclyl, wherein the 6-membered heterocyclyl is substituted with n independently selected R^{g} substituents, wherein n is an integer from 0-6, and R^{g} is —OH, halo, C₁₋₆alkyl, or C₁₋₆alkoxy, wherein the compound is of formula (I-D1):

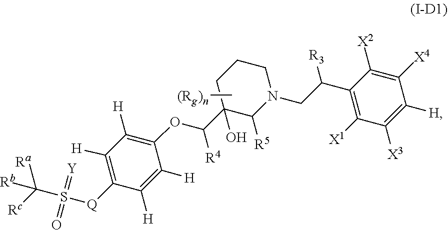

or a stereoisomer or tautomer thereof, or a pharmaceutically acceptable salt of any of the foregoing. In some variations, X¹, X², X³, X⁴, R³, R⁴, R⁵, R^{a}, R^{b}, R^{c}, R^{g}, R^{x}, Q, Y and n of formula (I-D1) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (I), such as a compound of formula (I-A2), or a pharmaceutically acceptable salt of any of the foregoing, wherein R^{x} is taken together with one of R¹ and R², and the atoms to which they are attached, to form a 6-membered heterocyclyl, wherein the 6-membered heterocyclyl is substituted with n independently selected R^{g} substituents, wherein n is an integer from 0-6, and R^{g} is —OH, halo, C₁₋₆alkyl, or C₁₋₆alkoxy, wherein the compound is of formula (I-D2):

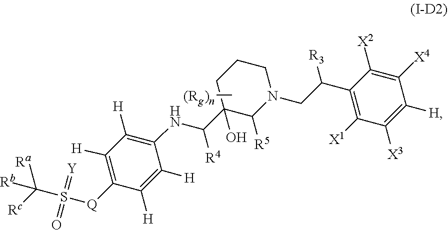

or a stereoisomer or tautomer thereof, or a pharmaceutically acceptable salt of any of the foregoing. In some variations, X¹, X², X³, X⁴, R³, R⁴, R⁵, R^{a}, R^{b}, R^{c}, R^{g}, R^{x}, Q, Y and n of formula (I-D2) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (I), such as a compound of formula (I-A3), or a pharmaceutically acceptable salt of any of the foregoing, wherein R^{x} is taken together with one of R¹ and R², and the atoms to which they are attached, to form a 6-membered heterocyclyl, wherein the 6-membered heterocyclyl is substituted with n independently selected R^{g} substituents, wherein n is an integer from 0-6, and R^{g} is —OH, halo, C₁₋₆alkyl, or C₁₋₆alkoxy, wherein the compound is of formula (I-D3):

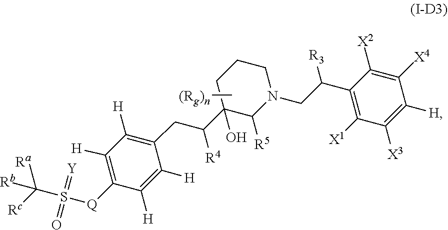

or a stereoisomer or tautomer thereof, or a pharmaceutically acceptable salt of any of the foregoing. In some variations, X¹, X², X³, X⁴, R³, R⁴, R⁵, R^{a}, R^{b}, R^{c}, R^{g}, R^{x}, Q, Y and n of formula (I-D3) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (I), such as a compound of formula (I-A1), or a pharmaceutically acceptable salt of any of the foregoing, wherein R^{x} is taken together with one of R¹ and R², and the atoms to which they are attached, to form a 6-membered heterocyclyl, wherein the 6-membered heterocyclyl is substituted with n independently selected R^{g} substituents, wherein n is an integer from 0-6, and R^{g} is —OH, halo, C₁₋₆alkyl, or C₁₋₆alkoxy, wherein the compound is of formula (I-E1):

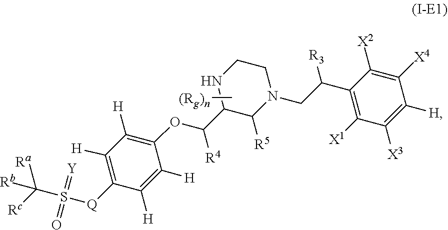

or a stereoisomer or tautomer thereof, or a pharmaceutically acceptable salt of any of the foregoing. In some variations, X¹, X², X³, X⁴, R³, R⁴, R⁵, R^{a}, R^{b}, R^{c}, R^{g}, R^{x}, Q, Y and n of formula (I-E1) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (I), such as a compound of formula (I-A2), or a pharmaceutically acceptable salt of any of the foregoing, wherein R^{x} is taken together with one of R¹ and R², and the atoms to which they are attached, to form a 6-membered heterocyclyl, wherein the 6-membered heterocyclyl is substituted with n independently selected R^{g} substituents, wherein n is an integer from 0-6, and R^{g} is —OH, halo, C₁₋₆alkyl, or C₁₋₆alkoxy, wherein the compound is of formula (I-E2):

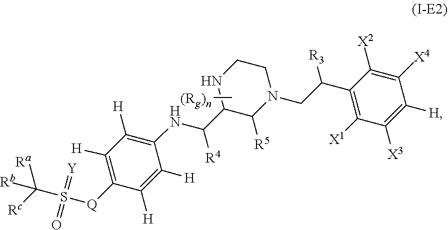

or a stereoisomer or tautomer thereof, or a pharmaceutically acceptable salt of any of the foregoing. In some variations, X¹, X², X³, X⁴, R³, R⁴, R⁵, R^{a}, R^{b}, R¹, R^{g}, R^{x}, Q, Y and n of formula (I-E2) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (I), such as a compound of formula (I-A3), or a pharmaceutically acceptable salt of any of the foregoing, wherein R^{x} is taken together with one of R¹ and R², and the atoms to which they are attached, to form a 6-membered heterocyclyl, wherein the 6-membered heterocyclyl is substituted with n independently selected R^{g} substituents, wherein n is an integer from 0-6, and R^{g} is —OH, halo, C₁₋₆alkyl, or C₁₋₆alkoxy, wherein the compound is of formula (I-E3):

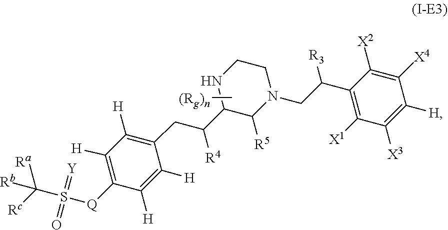

or a stereoisomer or tautomer thereof, or a pharmaceutically acceptable salt of any of the foregoing. In some variations, X¹, X², X³, X⁴, R³, R⁴, R⁵, R^{a}, R^{b}, R^{c}, R^{g}, R^{x}, Q, Y and n of formula (I-E3) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (I), such as a compound of formula (I-E1), (I-E2), or (I-E3), or a stereoisomer or tautomer thereof, or a pharmaceutically acceptable salt of any of the foregoing, wherein n is 0.

In some embodiments, provided herein is a compound of formula (A′) or formula (I), such as a compound of formula (I-E1), (I-E2), or (I-E3), or a stereoisomer or tautomer thereof, or a pharmaceutically acceptable salt of any of the foregoing, wherein n is 0 or 1. In some embodiments, n is 0. In some embodiments, n is 1.

In some embodiments, provided herein is a compound of formula (A) or formula (I), such as a compound of formula (I-A1), or a pharmaceutically acceptable salt of any of the foregoing, wherein R^{x} is taken together with one of R¹ and R², and the atoms to which they are attached, to form a 7-membered heterocyclyl, wherein the 7-membered heterocyclyl is substituted with n independently selected R^{g} substituents, wherein n is an integer from 0-6, and R^{g} is —OH, halo, C₁₋₆alkyl, or C₁₋₆alkoxy, wherein the compound is of formula (I-F1):

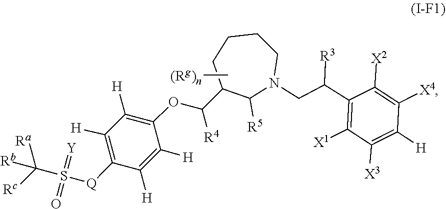

or a stereoisomer or tautomer thereof, or a pharmaceutically acceptable salt of any of the foregoing. In some variations, X¹, X², X³, X⁴, R³, R⁴, R⁵, R^{a}, R^{b}, R¹, R^{g}, R^{x}, Q, Y and n of formula (I-F1) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (I), such as a compound of formula (I-A2), or a pharmaceutically acceptable salt of any of the foregoing, wherein R^{x} is taken together with one of R¹ and R², and the atoms to which they are attached, to form a 7-membered heterocyclyl, wherein the 7-membered heterocyclyl is substituted with n independently selected R^{g} substituents, wherein n is an integer from 0-6, and R^{g} is —OH, halo, C₁₋₆alkyl, or C₁₋₆alkoxy, wherein the compound is of formula (I-F2):

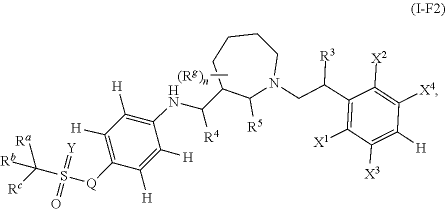

or a stereoisomer or tautomer thereof, or a pharmaceutically acceptable salt of any of the foregoing. In some variations, X¹, X², X³, X⁴, R³, R⁴, R⁵, R^{a}, R^{b}, R¹, R^{g}, R^{x}, Q, Y and n of formula (I-F2) are as defined for compound of formula (A′), or a stereoisomer or tautomer thereof, or a pharmaceutically acceptable salt of any of the foregoing, or any variation or embodiment thereof.

In some embodiments, provided herein is a compound of formula (A) or formula (I), such as a compound of formula (I-A3), or a pharmaceutically acceptable salt of any of the foregoing, wherein R^{x} is taken together with one of R¹ and R², and the atoms to which they are attached, to form a 7-membered heterocyclyl, wherein the 7-membered heterocyclyl is substituted with n independently selected R^{g} substituents, wherein n is an integer from 0-6, and R^{g} is —OH, halo, C₁₋₆alkyl, or C₁₋₆alkoxy, wherein the compound is of formula (I-F3):

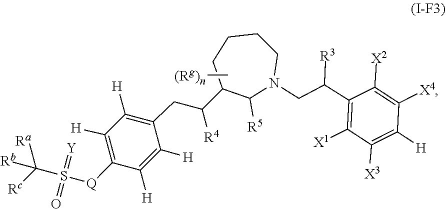

or a stereoisomer or tautomer thereof, or a pharmaceutically acceptable salt of any of the foregoing. In some variations, X¹, X², X³, X⁴, R³, R⁴, R⁵, R^{a}, R^{b}, R^{c}, R^{g}, R^{x}, Q, Y and n of formula (I-F3) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (I), such as a compound of formula (I-A1), or a pharmaceutically acceptable salt of any of the foregoing, wherein R^{x} is taken together with one of R¹ and R², and the atoms to which they are attached, to form a 7-membered heterocyclyl, wherein the 7-membered heterocyclyl is substituted with n independently selected R^{g} substituents, wherein n is an integer from 0-6, and R^{g} is —OH, halo, C₁₋₆alkyl, or C₁₋₆alkoxy, wherein the compound is of formula (I-G1):

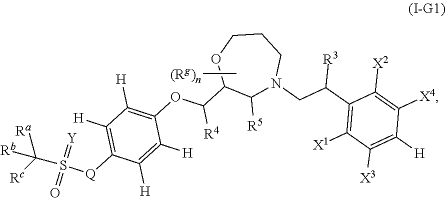

or a stereoisomer or tautomer thereof, or a pharmaceutically acceptable salt of any of the foregoing. In some variations, X¹, X², X³, X⁴, R³, R⁴, R⁵, R^{a}, R^{b}, R^{c}, R^{g}, R^{x}, Q, Y and n of formula (I-G1) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (I), such as a compound of formula (I-A2), or a pharmaceutically acceptable salt of any of the foregoing, wherein R^{x} is taken together with one of R¹ and R², and the atoms to which they are attached, to form a 7-membered heterocyclyl, wherein the 7-membered heterocyclyl is substituted with n independently selected R^{g} substituents, wherein n is an integer from 0-6, and R^{g} is —OH, halo, C₁₋₆alkyl, or C₁₋₆alkoxy, wherein the compound is of formula (I-G2):

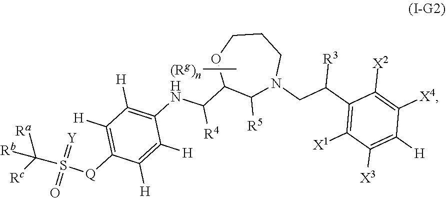

or a stereoisomer or tautomer thereof, or a pharmaceutically acceptable salt of any of the foregoing. In some variations, X¹, X², X³, X⁴, R³, R⁴, R⁵, R^{a}, R^{b}, R^{c}, R^{g}, R^{x}, Q, Y and n of formula (I-G2) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (I), such as a compound of formula (I-A3), or a pharmaceutically acceptable salt of any of the foregoing, wherein R^{x} is taken together with one of R¹ and R², and the atoms to which they are attached, to form a 7-membered heterocyclyl, wherein the 7-membered heterocyclyl is substituted with n independently selected R^{g} substituents, wherein n is an integer from 0-6, and R^{g} is —OH, halo, C₁₋₆alkyl, or C₁₋₆alkoxy, wherein the compound is of formula (I-G3):

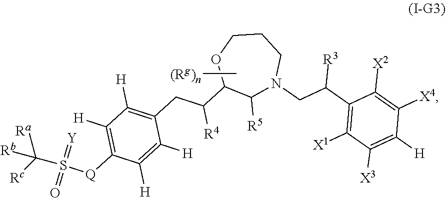

or a stereoisomer or tautomer thereof, or a pharmaceutically acceptable salt of any of the foregoing. In some variations, X¹, X², X³, X⁴, R³, R⁴, R⁵, R^{a}, R^{b}, R^{c}, R^{g}, R^{x}, Q, Y and n of formula (I-G3) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (I), such as a compound of formula (I-A1), (I-A2), (I-A3), (I-B1), (I-B2), (I-B3), (I-C1), (I-C2), (I-C3), (I-D1), (I-D2), (I-D3), (I-E1), (I-E2), (I-E3), (I-F1), (I-F2), (I-F3), (I-G1), (I-G2), or (I-G3), or a stereoisomer or tautomer thereof, or a pharmaceutically acceptable salt of any of the foregoing, wherein n is an integer from 0-6. In some embodiments, n is an integer from 0-5. In some embodiments, n is an integer from 0-4. In some embodiments, n is an integer from 0-3. In some embodiments, n is an integer from 0-2. In some embodiments, n is 0 or 1. In some embodiments, n is an integer from 1-6. In some embodiments, n is an integer from 1-5. In some embodiments, n is an integer from 1-4. In some embodiments, n is an integer from 1-3. In some embodiments, n is 1 or 2. In some embodiments, n is 0. In some embodiments, n is 1. In some embodiments, n is 2. In some variations, the embodiments provided herein also apply to a compound of formula (A′) or a stereoisomer or tautomer thereof, or a pharmaceutically acceptable salt of any of the foregoing, or any variation or embodiment thereof.

In some embodiments, provided herein is a compound of formula (A) or formula (I), such as a compound of formula (I-A1), (I-A2), (I-A3), (I-B1), (I-B2), (I-B3), (I-C1), (I-C2), (I-C3), (I-D1), (I-D2), (I-D3), (I-E1), (I-E2), (I-E3), (I-F1), (I-F2), (I-F3), (I-G1), (I-G2), or (I-G3), or a stereoisomer or tautomer thereof, or a pharmaceutically acceptable salt of any of the foregoing, wherein R^{g} is —OH, halo, C₁₋₆alkyl, or C₁₋₆alkoxy. In some embodiments, R⁹ is —OH, C₁₋₆alkyl, or C₁₋₆alkoxy. In some embodiments, R^{g} is —OH, halo, C₁₋₃alkyl, or C₁₋₃alkoxy. In some embodiments, R^{g} is —OH, methyl, or methoxy. In some embodiments, R^{g} is —OH. In some embodiments, R^{g} is C₁₋₆alkyl. In some embodiments, R^{g} is methyl. In some embodiments, R^{g} is C₁₋₆alkoxy. In some embodiments, R^{g} is methoxy. In some embodiments, R^{g} is halo. In some embodiments, R^{g} is fluoro. In some variations, the embodiments provided herein also apply to a compound of formula (A′) or a stereoisomer or tautomer thereof, or a pharmaceutically acceptable salt of any of the foregoing, or any variation or embodiment thereof.

In some embodiments, provided herein is a compound of formula (A) or formula (I), such as a compound of formula (I-A1), (I-A2), (I-A3), (I-B1), (I-B2), (I-B3), (I-C1), (I-C2), (I-C3), (I-D1), (I-D2), (I-D3), (I-E1), (I-E2), (I-E3), (I-F1), (I-F2), (I-F3), (I-G1), (I-G2), or (I-G3), or a stereoisomer or tautomer thereof, or a pharmaceutically acceptable salt of any of the foregoing, wherein n is 1 and R^{g} is C₁₋₆alkyl. In some embodiments, n is 1 and R^{g} is methyl. In some embodiments, n is 2 and each R⁹ is C₁₋₆alkyl. In some embodiments, n is 2 and each R⁹ is methyl. In some embodiments, n is 1 and R^{g} is —OH. In some embodiments, n is 1 and R^{g} is C₁₋₆alkoxy. In some embodiments, n is 1 and R^{g} is methoxy.

In some embodiments, provided herein is a compound of formula (A′) or formula (I), such as a compound of formula (I-A1), (I-A2), (I-A3), (I-B1), (I-B2), (I-B3), (I-C1), (I-C2), (I-C3), (I-D1), (I-D2), (I-D3), (I-E1), (I-E2), (I-E3), (I-F1), (I-F2), (I-F3), (I-G1), (I-G2), or (I-G3), or a stereoisomer or tautomer thereof, or a pharmaceutically acceptable salt of any of the foregoing, wherein n is 1 and R^{g} is C₁₋₆alkyl. In some embodiments, n is 1 and R^{g} is methyl. In some embodiments, n is 2 and each R⁹ is C₁₋₆alkyl. In some embodiments, n is 2 and each R⁹ is methyl. In some embodiments, n is 1 and R^{g} is —OH. In some embodiments, n is 1 and R^{g} is C₁₋₆alkoxy. In some embodiments, n is 1 and R^{g} is methoxy. In some embodiments, n is 2, one or R^{g} is methyl, and the other of R^{g} is —OH.

In some embodiments, provided herein is a compound of formula (A) or formula (I), such as a compound of formula (I-A1), (I-A2), (I-A3), (I-B1), (I-B2), (I-B3), (I-C1), (I-C2), (I-C3), (I-D1), (I-D2), (I-D3), (I-E1), (I-E2), (I-E3), (I-F1), (I-F2), (I-F3), (I-G1), (I-G2), or (I-G3), or a stereoisomer or tautomer thereof, or a pharmaceutically acceptable salt of any of the foregoing, wherein R⁴ and R⁵ are independently H. In some variations, the embodiments provided herein also apply to a compound of formula (A′) or a stereoisomer or tautomer thereof, or a pharmaceutically acceptable salt of any of the foregoing, or any variation or embodiment thereof.

In some embodiments, provided herein is a compound of formula (A) or formula (I), or a pharmaceutically acceptable salt of any of the foregoing, wherein R⁴ and R⁵ are taken, together with the atoms to which they are attached, to form a C₃₋₈cycloalkyl. In some embodiments, R⁴ and R⁵ are taken, together with the atoms to which they are attached, to form a C₃₋₆cycloalkyl. In some embodiments, R⁴ and R⁵ are taken, together with the atoms to which they are attached, to form cyclobutyl. In some variations, the embodiments provided herein also apply to a compound of formula (A′) or a stereoisomer or tautomer thereof, or a pharmaceutically acceptable salt of any of the foregoing, or any variation or embodiment thereof.

In some embodiments, provided herein is a compound of formula (A) or formula (I), such as a compound of formula (I-A1), or a pharmaceutically acceptable salt of any of the foregoing, wherein R⁴ and R⁵ are taken, together with the atoms to which they are attached, to form cyclobutyl, wherein the compound is of formula (I-H1):

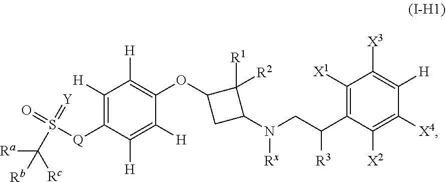

or a stereoisomer or tautomer thereof, or a pharmaceutically acceptable salt of any of the foregoing. In some variations, X¹, X², X³, X⁴, R³, R⁴, R⁵, R^{a}, R^{b}, R^{c}, R^{x}, Q, and Y of formula (I-H1) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (I), such as a compound of formula (I-A2), or a pharmaceutically acceptable salt of any of the foregoing, wherein R⁴ and R⁵ are taken, together with the atoms to which they are attached, to form cyclobutyl, wherein the compound is of formula (I-H2):

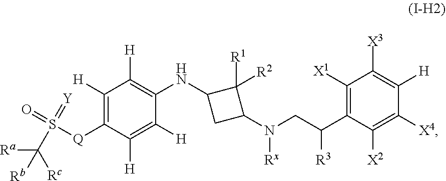

or a stereoisomer or tautomer thereof, or a pharmaceutically acceptable salt of any of the foregoing. In some variations, X¹, X², X³, X⁴, R³, R⁴, R⁵, R^{a}, R^{b}, R^{c}, R^{x}, Q, and Y of formula (I-H2) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (I), such as a compound of formula (I-A3), or a pharmaceutically acceptable salt of any of the foregoing, wherein R⁴ and R⁵ are taken, together with the atoms to which they are attached, to form cyclobutyl, wherein the compound is of formula (I-H3):

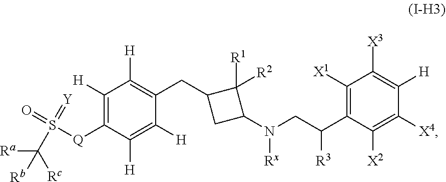

or a stereoisomer or tautomer thereof, or a pharmaceutically acceptable salt of any of the foregoing. In some variations, X¹, X², X³, X⁴, R³, R⁴, R⁵, R^{a}, R^{b}, R^{c}, R^{x}, Q, and Y of formula (I-H3) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (I), such as a compound of formula (I-H1), (I-H2), or (I-H3), or a stereoisomer or tautomer thereof, or a pharmaceutically acceptable salt of any of the foregoing, wherein R¹ and R² are each H. In some embodiments, provided herein is a compound of formula (A) or formula (I), such as a compound of formula (I-H1), (I-H2), or (I-H3), or a stereoisomer or tautomer thereof, or a pharmaceutically acceptable salt of any of the foregoing, wherein R^{x} is H. In some embodiments, R¹, R², and R^{x} are each H. In some embodiments, the embodiments provided herein also apply to a compound of formula (A′) or a stereoisomer or tautomer thereof, or a pharmaceutically acceptable salt of any of the foregoing, or any variation or embodiment thereof.

In some embodiments, provided herein is a compound of formula (A) or formula (I), such as a compound of formula (I-A1), (I-A2), (I-A3), (I-B1), (I-B2), (I-B3), (I-C1), (I-C2), (I-C3), (I-D1), (I-D2), (I-D3), (I-E1), (I-E2), (I-E3), (I-F1), (I-F2), (I-F3), (I-G1), (I-G2), (I-G3), (I-H1), (I-H2), or (I-H3), or a stereoisomer or tautomer thereof, or a pharmaceutically acceptable salt of any of the foregoing, wherein R³ is H, —OH, halo, or C₁₋₆alkoxy. In some embodiments, R³ is H. In some embodiments, R³ is —OH, halo, or C₁₋₆alkoxy. In some embodiments, R³ is H or —OH. In some embodiments, R³ is —OH. In some embodiments, R³ is halo. In some embodiments, R³ is C₁₋₆alkoxy. In some embodiments, R³ is methoxy.

In some embodiments, provided herein is a compound of formula (A) or formula (I), such as a compound of formula (I-A1), (I-A2), (I-A3), (I-B1), (I-B2), (I-B3), (I-C1), (I-C2), (I-C3), (I-D1), (I-D2), (I-D3), (I-E1), (I-E2), (I-E3), (I-F1), (I-F2), (I-F3), (I-G1), (I-G2), or (I-G3), or a stereoisomer or tautomer thereof, or a pharmaceutically acceptable salt of any of the foregoing, wherein R³, R⁴, and R⁵ are each H. In some embodiments, provided herein is a compound of formula (A) or formula (I), such as a compound of formula (I-H1), (I-H2), or (I-H3), or a stereoisomer or tautomer thereof, or a pharmaceutically acceptable salt of any of the foregoing, wherein R¹, R², R³, and R^{x} are each H. In some embodiments, the embodiments provided herein also apply to a compound of formula (A′) or a stereoisomer or tautomer thereof, or a pharmaceutically acceptable salt of any of the foregoing, or any variation or embodiment thereof. In some embodiments, provided herein is a compound of formula (A), or a stereoisomer or tautomer thereof, or a pharmaceutically acceptable salt of any of the foregoing, wherein L is

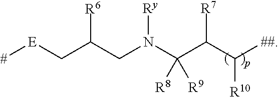

In some embodiments, L is

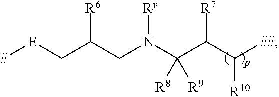

and the compound of formula (A) is a compound of formula (II):

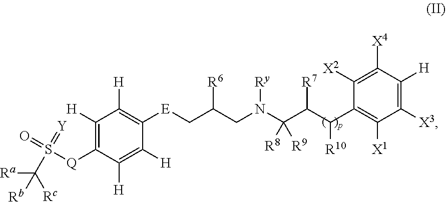

or a stereoisomer or tautomer thereof, or a pharmaceutically acceptable salt of any of the foregoing. In some variations, X¹, X², X³, X⁴, R⁶, R⁷, R⁸, R⁹, R¹⁰, R^{a}, R^{b}, R^{c}, R^{y}, p, E, Q, and Y of formula (II) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (II), or a stereoisomer or tautomer thereof, or a pharmaceutically acceptable salt of any of the foregoing, wherein E is O, wherein the compound is of formula (II-A1):

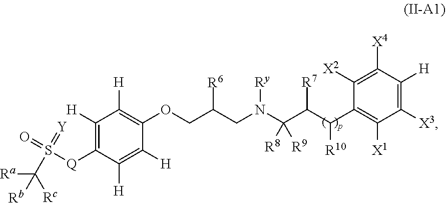

or a stereoisomer or tautomer thereof, or a pharmaceutically acceptable salt of any of the foregoing. In some variations, X¹, X², X³, X⁴, R⁶, R⁷, R⁸, R⁹, R¹⁰, R^{a}, R^{b}, R^{c}, R^{y}, p, Q, and Y of formula (II-A1) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (II), or a stereoisomer or tautomer thereof, or a pharmaceutically acceptable salt of any of the foregoing, wherein E is NH, wherein the compound is of formula (II-A2):

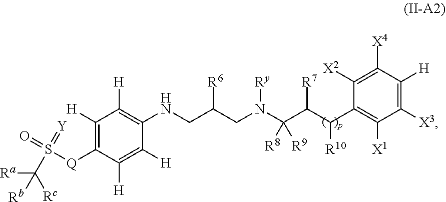

or a stereoisomer or tautomer thereof, or a pharmaceutically acceptable salt of any of the foregoing. In some embodiments, E is N(C₁₋₆alkyl). In some embodiments, E is N(CH₃). In some variations, X¹, X², X³, X⁴, R⁶, R⁷, R⁸, R⁹, R¹⁰, R^{a}, R^{b}, R^{c}, R^{y}, p, Q, and Y of formula (II-A2) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (II), or a stereoisomer or tautomer thereof, or a pharmaceutically acceptable salt of any of the foregoing, wherein E is CH₂, wherein the compound is of formula (II-A3):

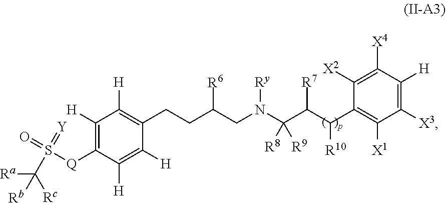

or a stereoisomer or tautomer thereof, or a pharmaceutically acceptable salt of any of the foregoing. In some embodiments, E is CH(C₁₋₆alkyl). In some embodiments, E is CH(CH₃). In some variations, X¹, X², X³, X⁴, R⁶, R⁷, R⁸, R⁹, R¹⁰, R^{a}, R^{b}, Re, R^{y}, p, Q, and Y of formula (II-A3) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (II), or a stereoisomer or tautomer thereof, or a pharmaceutically acceptable salt of any of the foregoing, wherein R^{y} is taken together with R⁷, and the atoms to which they are attached, to form a 3-8 membered heterocyclyl. In some embodiments, R^{y} is taken together with R⁷, and the atoms to which they are attached, to form a 3-6 membered heterocyclyl. In some embodiments, R^{y} is taken together with R⁷, and the atoms to which they are attached, to form a 5-8 membered heterocyclyl. In some embodiments, R^{y} is taken together with R⁷, and the atoms to which they are attached, to form a 5-6 membered heterocyclyl. In some embodiments, R^{y} is taken together with R⁷, and the atoms to which they are attached, to form a 5-membered heterocyclyl. In some embodiments, R^{y} is taken together with R⁷, and the atoms to which they are attached, to form a 6-membered heterocyclyl. In some variations, the embodiments provided herein also apply to a compound of formula (A′) or a stereoisomer or tautomer thereof, or a pharmaceutically acceptable salt of any of the foregoing, or any variation or embodiment thereof.

In some embodiments of the foregoing, provided herein is a compound of formula (A) or formula (II), such as a compound of formula (II-A1), (II-A2), or (II-A3), or a stereoisomer or tautomer thereof, or a pharmaceutically acceptable salt of any of the foregoing, wherein p is 1. In other embodiments, provided herein is a compound of formula (A) or formula (II), such as a compound of formula (II-A1), (II-A2), or (II-A3), or a stereoisomer or tautomer thereof, or a pharmaceutically acceptable salt of any of the foregoing, wherein p is 0. In some variations, the embodiments provided herein also apply to a compound of formula (A′) or a stereoisomer or tautomer thereof, or a pharmaceutically acceptable salt of any of the foregoing, or any variation or embodiment thereof.

In some embodiments, provided herein is a compound of formula (A) or formula (II), such as a compound of formula (II-A1), or a stereoisomer or tautomer thereof, or a pharmaceutically acceptable salt of any of the foregoing, wherein R^{y} is taken together with R⁷, and the atoms to which they are attached, to form a 6-membered heterocyclyl, and p is 1, wherein the compound is of formula (II-B1):

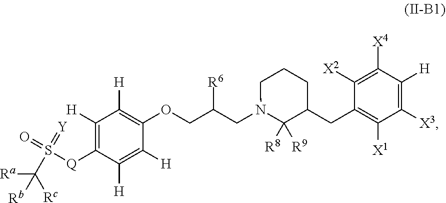

or a stereoisomer or tautomer thereof, or a pharmaceutically acceptable salt of any of the foregoing. In some variations, X¹, X², X³, X⁴, R⁶, R⁸, R⁹, R¹⁰, R^{a}, R^{b}, R^{c}, Q, and Y of formula (II-B1) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (II), such as a compound of formula (II-A1), or a stereoisomer or tautomer thereof, or a pharmaceutically acceptable salt of any of the foregoing, wherein R^{y} is taken together with R⁷, and the atoms to which they are attached, to form a 5-membered heterocyclyl, and p is 0, wherein the compound is of formula (II-C1):

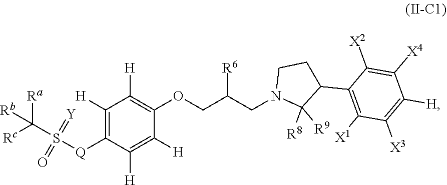

or a stereoisomer or tautomer thereof, or a pharmaceutically acceptable salt of any of the foregoing. In some variations, X¹, X², X³, X⁴, R⁶, R⁸, R⁹, R¹⁰, R^{a}, R^{b}, R^{c}, Q, and Y of formula (II-C1) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (II), such as a compound of formula (II-A2), or a stereoisomer or tautomer thereof, or a pharmaceutically acceptable salt of any of the foregoing, wherein R^{y} is taken together with R⁷, and the atoms to which they are attached, to form a 5-membered heterocyclyl, and p is 0, wherein the compound is of formula (II-C2):

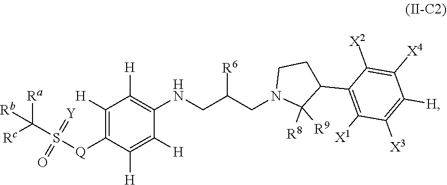

or a stereoisomer or tautomer thereof, or a pharmaceutically acceptable salt of any of the foregoing. In some variations, X¹, X², X³, X⁴, R⁶, R⁸, R⁹, R¹⁰, R^{a}, R^{b}, R^{c}, Q, and Y of formula (II-C2) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (II), such as a compound of formula (II-A3), or a stereoisomer or tautomer thereof, or a pharmaceutically acceptable salt of any of the foregoing, wherein R^{y} is taken together with R⁷, and the atoms to which they are attached, to form a 5-membered heterocyclyl, and p is 0, wherein the compound is of formula (II-C3):

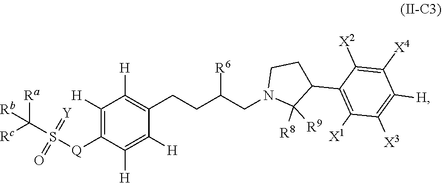

or a stereoisomer or tautomer thereof, or a pharmaceutically acceptable salt of any of the foregoing. In some variations, X¹, X², X³, X⁴, R⁶, R⁸, R⁹, R¹⁰, R^{a}, R^{b}, R^{c}, Q, and Y of formula (II-C3) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (II), such as a compound of formula (II-A1), or a stereoisomer or tautomer thereof, or a pharmaceutically acceptable salt of any of the foregoing, wherein R^{y} is taken together with R⁷, and the atoms to which they are attached, to form a 6-membered heterocyclyl, and p is 0, wherein the compound is of formula (II-D1):

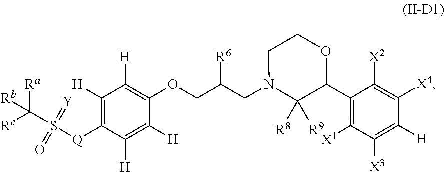

or a stereoisomer or tautomer thereof, or a pharmaceutically acceptable salt of any of the foregoing. In some variations, X¹, X², X³, X⁴, R⁶, R⁸, R⁹, R¹⁰, R^{a}, R^{b}, R^{c}, Q, and Y of formula (II-D1) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (II), such as a compound of formula (II-A2), or a stereoisomer or tautomer thereof, or a pharmaceutically acceptable salt of any of the foregoing, wherein R^{y} is taken together with R⁷, and the atoms to which they are attached, to form a 6-membered heterocyclyl, and p is 0, wherein the compound is of formula (II-D2):

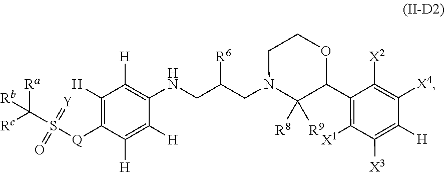

or a stereoisomer or tautomer thereof, or a pharmaceutically acceptable salt of any of the foregoing. In some variations, X¹, X², X³, X⁴, R⁶, R⁸, R⁹, R¹⁰, R^{a}, R^{b}, R^{c}, Q, and Y of formula (II-D2) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (II), such as a compound of formula (II-A3), or a stereoisomer or tautomer thereof, or a pharmaceutically acceptable salt of any of the foregoing, wherein R^{y} is taken together with R⁷, and the atoms to which they are attached, to form a 6-membered heterocyclyl, and p is 0, wherein the compound is of formula (II-D3):

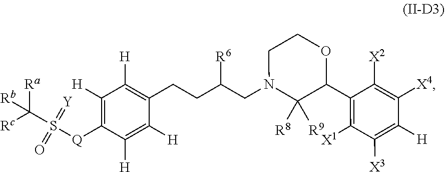

or a stereoisomer or tautomer thereof, or a pharmaceutically acceptable salt of any of the foregoing. In some variations, X¹, X², X³, X⁴, R⁶, R⁸, R⁹, R¹⁰, R^{a}, R^{b}, R^{c}, Q, and Y of formula (II-D3) are as defined for a compound of formula (A′), or a stereoisomer or tautomer thereof, or a pharmaceutically acceptable salt of any of the foregoing, or any variation or embodiment thereof.

In some embodiments of the foregoing, provided herein is a compound of formula (A) or formula (II), such as a compound of formula (II-A1), (II-A2), (II-A3), (II-B1), (II-C1), (II-C2), (II-C3), (II-D1), (II-D2), or (II-D3), or a stereoisomer or tautomer thereof, or a pharmaceutically acceptable salt of any of the foregoing, wherein R⁶ is —OH. In some embodiments, R⁶ is —OH, R⁸ is C₁₋₆alkyl, and R^{g} is C₁₋₆alkyl. In some embodiments, R⁶ is —OH, R⁸ is methyl, and R^{g} is methyl. In some embodiments, R⁶ is —OH, R⁸ is H, and R^{g} is H. In some variations, the embodiments provided herein also apply to a compound of formula (A′) or a stereoisomer or tautomer thereof, or a pharmaceutically acceptable salt of any of the foregoing, or any variation or embodiment thereof.

In some embodiments, provided herein is a compound of formula (A) or formula (II), or a stereoisomer or tautomer thereof, or a pharmaceutically acceptable salt of any of the foregoing, wherein one of R⁸ and R⁹ is taken together with R¹⁰, and the atoms to which they are attached, to form a C₃₋₈cycloalkyl. In some embodiments, one of R⁸ and R⁹ is taken together with R¹⁰, and the atoms to which they are attached, to form a C₃₋₆cycloalkyl. In some embodiments, one of R⁸ and R⁹ is taken together with R¹⁰, and the atoms to which they are attached, to form a C₃₋₄cycloalkyl. In some embodiments, one of R⁸ and R⁹ is taken together with R¹⁰, and the atoms to which they are attached, to form cyclobutyl. In some variations, the embodiments provided herein also apply to a compound of formula (A′) or a stereoisomer or tautomer thereof, or a pharmaceutically acceptable salt of any of the foregoing, or any variation or embodiment thereof.

In some embodiments, provided herein is a compound of formula (A) or formula (II), or a stereoisomer or tautomer thereof, or a pharmaceutically acceptable salt of any of the foregoing, wherein one of R⁸ and R⁹ is taken together with R¹⁰, and the atoms to which they are attached, to form cyclobutyl, wherein the compound is of formula (II-E1):

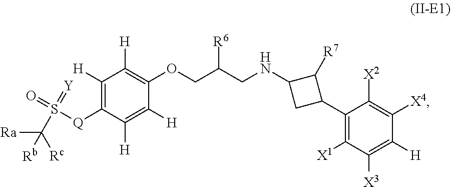

or a stereoisomer or tautomer thereof, or a pharmaceutically acceptable salt of any of the foregoing. In some variations, X¹, X², X³, X⁴, R⁶, R⁸, R⁹, R¹⁰, R^{a}, R^{b}, R^{c}, Q, and Y of formula (II-E1) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (II), such as a compound of formula (II-E1), or a stereoisomer or tautomer thereof, or a pharmaceutically acceptable salt of any of the foregoing, wherein R⁶ is —OH and R⁷ is H. In some variations, the embodiments provided herein also apply to a compound of formula (A′) or a stereoisomer or tautomer thereof, or a pharmaceutically acceptable salt of any of the foregoing, or any variation or embodiment thereof.

In some embodiments, provided herein is a compound of formula (A), or a stereoisomer or tautomer thereof, or a pharmaceutically acceptable salt of any of the foregoing, wherein L is

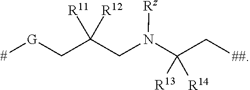

In some embodiments, provided herein is a compound of formula (A), or a stereoisomer or tautomer thereof, or a pharmaceutically acceptable salt of any of the foregoing, wherein L is

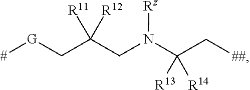

wherein the compound is of formula (III):

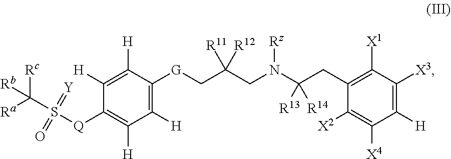

or a stereoisomer or tautomer thereof, or a pharmaceutically acceptable salt of any of the foregoing. In some variations, X¹, X², X³, X⁴, R¹¹, R¹², R¹³, R¹⁴, R^{a}, R^{b}, R^{c}, R^{z}, G, Q, and Y of formula (III) are as defined for a compound of formula (A′) or a stereoisomer or tautomer thereof, or a pharmaceutically acceptable salt of any of the foregoing, or any variation or embodiment thereof.

In some embodiments, provided herein is a compound of formula (A) or formula (III), or a stereoisomer or tautomer thereof, or a pharmaceutically acceptable salt of any of the foregoing, wherein G is O, wherein the compound is of formula (III-A1):

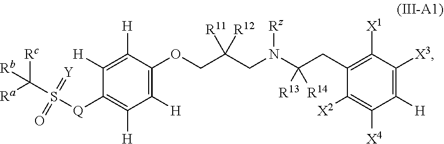

or a stereoisomer or tautomer thereof, or a pharmaceutically acceptable salt of any of the foregoing. In some variations, X¹, X², X³, X⁴, R¹¹, R¹², R¹³, R¹⁴, R^{a}, R^{b}, R^{c}, R^{z}, Q, and Y of formula (III-A1) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (III), or a stereoisomer or tautomer thereof, or a pharmaceutically acceptable salt of any of the foregoing, wherein G is NH, wherein the compound is of formula (III-A2):

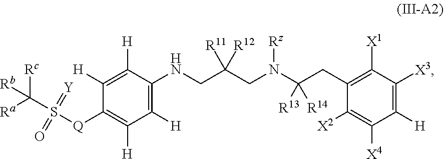

or a stereoisomer or tautomer thereof, or a pharmaceutically acceptable salt of any of the foregoing. In some embodiments, G is N(C₁₋₆alkyl). In some embodiments, G is N(CH₃). In some variations, X¹, X², X³, X⁴, R¹¹, R¹², R¹³, R¹⁴, R^{a}, R^{b}, Re, R^{z}, Q, and Y of formula (III-A2) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (III), or a stereoisomer or tautomer thereof, or a pharmaceutically acceptable salt of any of the foregoing, wherein G is CH₂, wherein the compound is of formula (III-A3):

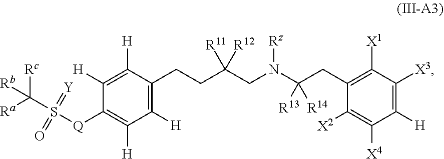

or a stereoisomer or tautomer thereof, or a pharmaceutically acceptable salt of any of the foregoing. In some embodiments, G is CH(C₁₋₆alkyl). In some embodiments, G is CH(CH₃). In some variations, X¹, X², X³, X⁴, R¹¹, R¹², R¹³, R¹⁴, R^{a}, R^{b}, Re, R^{z}, Q, and Y of formula (III-A3) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (III), such as a compound of formula (III-A1), (III-A2), or (III-A3), or a stereoisomer or tautomer thereof, or a pharmaceutically acceptable salt of any of the foregoing, wherein R^{z} is H. In some embodiments, R^{z} is C₁₋₆alkyl, wherein the C₁₋₆alkyl is optionally substituted with one or more C₃₋₈cycloalkyl. In some embodiments, R^{z} is unsubstituted C₁₋₆alkyl. In some embodiments, R^{z} is unsubstituted C₁₋₃alkyl. In some embodiments, R^{z} is unsubstituted methyl or unsubstituted ethyl. In some embodiments, R^{z} is unsubstituted ethyl. In some embodiments, R^{z} is C₁₋₆alkyl, wherein the C₁₋₆alkyl is substituted with one or more C₃₋₈cycloalkyl. In some embodiments, R^{z} is C₁₋₆alkyl, wherein the C₁₋₆alkyl is substituted with one or more C₃₋₆cycloalkyl. In some embodiments, R^{z} is methyl, wherein the methyl is substituted with cyclopropyl. In some variations, the embodiments provided herein also apply to a compound of formula (A′) or a stereoisomer or tautomer thereof, or a pharmaceutically acceptable salt of any of the foregoing, or any variation or embodiment thereof.

In some embodiments, provided herein is a compound of formula (A) or formula (III), such as a compound of formula (III-A1), (III-A2), or (III-A3), or a stereoisomer or tautomer thereof, or a pharmaceutically acceptable salt of any of the foregoing, wherein one of R¹¹ and R¹² is —OH and the other of R¹¹ and R¹² is H, halo, or C₁₋₆alkyl. In some embodiments, one of R¹¹ and R¹² is —OH and the other of R¹¹ and R¹² is H. In some embodiments, one of R¹¹ and R¹² is —OH and the other of R¹¹ and R¹² is C₁₋₆alkyl. In some embodiments, one of R¹¹ and R¹² is —OH and the other of R¹¹ and R¹² is methyl. In some variations, the embodiments provided herein also apply to a compound of formula (A′) or a stereoisomer or tautomer thereof, or a pharmaceutically acceptable salt of any of the foregoing, or any variation or embodiment thereof.

In some embodiments, provided herein is a compound of formula (A) or formula (III), such as a compound of formula (III-A1), (III-A2), or (III-A3), or a stereoisomer or tautomer thereof, or a pharmaceutically acceptable salt of any of the foregoing, wherein R¹³ and R¹⁴ are both H. In some embodiments, R¹³ and R¹⁴ are both C₁₋₆alkyl. In some embodiments, R¹³ and R¹⁴ are both methyl. In some embodiments, one of R¹³ and R¹⁴ is H and the other of R¹³ and R¹⁴ is C₁₋₆alkyl or C₃₋₈-cycloalkyl. In some embodiments, one of R¹³ and R¹⁴ is H and the other of R¹³ and R¹⁴ is C₁₋₆alkyl. In some embodiments, one of R¹³ and R¹⁴ is H and the other of R¹³ and R¹⁴ is methyl. In some embodiments, one of R¹³ and R¹⁴ is H and the other of R¹³ and R¹⁴ is C₃₋₈-cycloalkyl. In some embodiments, one of R¹³ and R¹⁴ is H and the other of R¹³ and R¹⁴ is cyclopropyl. In some embodiments, R¹³ and R¹⁴ are taken, together with the atoms to which they are attached, to form a 3-8 membered heterocyclyl. In some embodiments, R¹³ and R¹⁴ are taken, together with the atoms to which they are attached, to form a 3-6 membered heterocyclyl. In some embodiments, R¹³ and R¹⁴ are taken, together with the atoms to which they are attached, to form a 5-8 membered heterocyclyl. In some embodiments, R¹³ and R¹⁴ are taken, together with the atoms to which they are attached, to form a 5-6 membered heterocyclyl. In some embodiments, R¹³ and R¹⁴ are taken, together with the atoms to which they are attached, to form tetrahydrofuranyl. In some variations, the embodiments provided herein also apply to a compound of formula (A′) or a stereoisomer or tautomer thereof, or a pharmaceutically acceptable salt of any of the foregoing, or any variation or embodiment thereof.

In some embodiments, provided herein is a compound of formula (A), such as a compound of formula (I), (I-A1), (I-A2), (I-A3), (I-B1), (I-B2), (I-B3), (I-C1), (I-C2), (I-C3), (I-D1), (I-D2), (I-D3), (I-E1), (I-E2), (I-E3), (I-F1), (I-F2), (I-F3), (I-G1), (I-G2), (I-G3), (I-H-1), (I-H-2), (I-H-3), (II), (II-A1), (II-A2), (II-A3), (II-B1), (II-C1), (II-C2), (II-C3), (II-D1), (II-D2), (II-D3), (II-E1), (III), (III-A1), (III-A2), or (III-A3), or a stereoisomer or tautomer thereof, or a pharmaceutically acceptable salt of any of the foregoing, wherein X¹, X², X³, and X⁴ are, independently of each other, H, halo, —CN, C₁₋₆alkyl, or C₁₋₆alkoxy, wherein the C₁₋₆alkyl or C₁₋₆alkoxy is optionally substituted with one or more halo, provided that at least one of X¹, X², X³, and X⁴ is halo, —CN, C₁₋₆alkyl, or C₁₋₆alkoxy, wherein the C₁₋₆alkyl or C₁₋₆alkoxy is optionally substituted with one or more halo. In some embodiments, three of X¹, X², X³, and X⁴ are H and one of X¹, X², X³, and X⁴ is halo, —CN, C₁₋₆alkyl, or C₁₋₆alkoxy, wherein the C₁₋₆alkyl or C₁₋₆alkoxy is optionally substituted with one or more halo. In some embodiments, two of X¹, X², X³, and X⁴ are H and two of X¹, X², X³, and X⁴ are independently halo, —CN, C₁₋₆alkyl, or C₁₋₆alkoxy, wherein the C₁₋₆alkyl or C₁₋₆alkoxy is optionally substituted with one or more halo.

In some embodiments, provided herein is a compound of formula (A′), such as a compound of formula (I), (I-A1), (I-A2), (I-A3), (I-B1), (I-B2), (I-B3), (I-C1), (I-C2), (I-C3), (I-D1), (I-D2), (I-D3), (I-E1), (I-E2), (I-E3), (I-F1), (I-F2), (I-F3), (I-G1), (I-G2), (I-G3), (I-H1), (I-H-2), (I-H-3), (II), (II-A1), (II-A2), (II-A3), (II-B1), (II-C1), (II-C2), (II-C3), (II-D1), (II-D2), (II-D3), (II-E1), (III), (III-A1), (III-A2), or (III-A3), or a stereoisomer or tautomer thereof, or a pharmaceutically acceptable salt of any of the foregoing, wherein X¹, X², X³, and X⁴ are, independently of each other, H, halo, —CN, C₁₋₆alkyl, C₁₋₆alkoxy, or SF⁵, wherein the C₁₋₆alkyl or C₁₋₆alkoxy is optionally substituted with one or more halo, provided that at least one of X¹, X², X³, and X⁴ is halo, —CN, C₁₋₆alkyl, C₁₋₆alkoxy, or SF⁵, wherein the C₁₋₆alkyl or C₁₋₆alkoxy is optionally substituted with one or more halo. In some embodiments, three of X¹, X², X³, and X⁴ are H and one of X¹, X², X³, and X⁴ is halo, —CN, C₁₋₆alkyl, C₁₋₆alkoxy, or SF⁵, wherein the C₁₋₆alkyl or C₁₋₆alkoxy is optionally substituted with one or more halo. In some embodiments, two of X¹, X², X³, and X⁴ are H and two of X¹, X², X³, and X⁴ are independently halo, —CN, C₁₋₆alkyl, C₁₋₆alkoxy, or SF⁵, wherein the C₁₋₆alkyl or C₁₋₆alkoxy is optionally substituted with one or more halo.

In some embodiments, provided herein is a compound of formula (A), such as a compound of formula (I), (I-A1), (I-A2), (I-A3), (I-B1), (I-B2), (I-B3), (I-C1), (I-C2), (I-C3), (I-D1), (I-D2), (I-D3), (I-E1), (I-E2), (I-E3), (I-F1), (I-F2), (I-F3), (I-G1), (I-G2), (I-G3), (I-H-1), (I-H-2), (I-H-3), (II), (II-A1), (II-A2), (II-A3), (II-B1), (II-C1), (II-C2), (II-C3), (II-D1), (II-D2), (II-D3), (II-E1), (III), (III-A1), (III-A2), or (III-A3), or a stereoisomer or tautomer thereof, or a pharmaceutically acceptable salt of any of the foregoing, wherein X¹ and X² are each H, one of X³ and X⁴ is H, and the other of X³ and X⁴ is halo. In some embodiments, X¹ and X² are each H, one of X³ and X⁴ is H, and the other of X³ and X⁴ is chloro. In some variations, the embodiments provided herein also apply to a compound of formula (A′) or a stereoisomer or tautomer thereof, or a pharmaceutically acceptable salt of any of the foregoing, or any variation or embodiment thereof. In some embodiments, provided herein is a compound of formula (A), such as a compound of formula (I), (I-A1), (I-A2), (I-A3), (I-B1), (I-B2), (I-B3), (I-C1), (I-C2), (I-C3), (I-D1), (I-D2), (I-D3), (I-E1), (I-E2), (I-E3), (I-F1), (I-F2), (I-F3), (I-G1), (I-G2), (I-G3), (I-H-1), (I-H-2), (I-H-3), (II), (II-A1), (II-A2), (II-A3), (II-B1), (II-C1), (II-C2), (II-C3), (II-D1), (II-D2), (II-D3), (II-E1), (III), (III-A1), (III-A2), or (III-A3), or a stereoisomer or tautomer thereof, or a pharmaceutically acceptable salt of any of the foregoing, wherein one of X¹ and X² is H and the other of X¹ and X² is C₁₋₆alkyl, and X³ and X⁴ are each H. In some embodiments, one of X¹ and X² is H and the other of X¹ and X² is ethyl, and X³ and X⁴ are each H. In some embodiments, one of X¹ and X² is H and the other of X¹ and X² is C₁₋₆alkoxy, wherein the C₁₋₆alkoxy is optionally substituted with one or more halo, and X³ and X⁴ are each H. In some embodiments, one of X¹ and X² is H and the other of X¹ and X² is methoxy, and X³ and X⁴ are each H. In some embodiments, one of X¹ and X² is H and the other of X¹ and X² is —O—CHF₂, and X³ and X⁴ are each H. In some embodiments, one of X¹ and X² is H and the other of X¹ and X² is halo, and one of X³ and X⁴ is H and the other of X³ and X⁴ is halo. In some embodiments, one of X¹ and X² is H and the other of X¹ and X² is chloro, and one of X³ and X⁴ is H and the other of X³ and X⁴ is chloro. In some embodiments, X¹ and X² are each H, and X³ and X⁴ are each halo. In some embodiments, X¹ and X² are each H, and X³ and X⁴ are each chloro. In some embodiments, X¹ and X² are each H, and one of X³ and X⁴ is H and the other of X³ and X⁴ is —CN. In some embodiments, provided herein is a compound of formula (A), such as a compound of formula (I), (I-A1), (I-A2), (I-A3), (I-B1), (I-B2), (I-B3), (I-C1), (I-C2), (I-C3), (I-D1), (I-D2), (I-D3), (I-E1), (I-E2), (I-E3), (I-F1), (I-F2), (I-F3), (I-G1), (I-G2), (I-G3), (I-H1), (I-H2), (I-H-3), (II), (II-A1), (II-A2), (II-A3), (II-B1), (II-C1), (II-C2), (II-C3), (II-D1), (II-D2), (II-D3), (II-E1), (III), (III-A1), (III-A2), or (III-A3), or a stereoisomer or tautomer thereof, or a pharmaceutically acceptable salt of any of the foregoing, wherein one of X¹ and X² is H and the other of X¹ and X² is C₁₋₆alkyl, and X³ and X⁴ are each H. In some embodiments, one of X¹ and X² is H and the other of X¹ and X² is ethyl, and X³ and X⁴ are each H. In some embodiments, one of X¹ and X² is H and the other of X¹ and X² is C₁₋₆alkoxy, wherein the C₁₋₆alkoxy is optionally substituted with one or more halo, and X³ and X⁴ are each H. In some embodiments, one of X¹ and X² is H and the other of X¹ and X² is methoxy, and X³ and X⁴ are each H. In some embodiments, one of X¹ and X² is H and the other of X¹ and X² is —O—CHF₂, and X³ and X⁴ are each H. In some embodiments, one of X¹ and X² is H and the other of X¹ and X² is —O—CHF₂, and one of X³ and X⁴ is H and the other of X³ and X⁴ is chloro. In some embodiments, one of X¹ and X² is H and the other of X¹ and X² is halo, and one of X³ and X⁴ is H and the other of X³ and X⁴ is halo. In some embodiments, one of X¹ and X² is H and the other of X¹ and X² is C₁₋₆alkyl, and one of X³ and X⁴ is H and the other of X³ and X⁴ is halo. In some embodiments, one of X¹ and X² is H and the other of X¹ and X² is methyl, and one of X³ and X⁴ is H and the other of X³ and X⁴ is halo. In some embodiments, one of X¹ and X² is H and the other of X¹ and X² is C₁₋₆alkoxy, one of X³ and X⁴ is H and the other of X³ and X⁴ is halo. In some embodiments, one of X¹ and X² is H and the other of X¹ and X² is methoxy, one of X³ and X⁴ is H and the other of X³ and X⁴ is halo. In some embodiments, one of X¹ and X² is H and the other of X¹ and X² is chloro, and one of X³ and X⁴ is H and the other of X³ and X⁴ is chloro. In some embodiments, X¹ and X² are each H, and X³ and X⁴ are each halo. In some embodiments, X¹ and X² are each H, and X³ and X⁴ are each chloro. In some embodiments, X¹ and X² are each H, one of X³ and X⁴ is chloro and the other of X³ and X⁴ is fluoro. In some embodiments, X¹ and X² are each H, and one of X³ and X⁴ is H and the other of X³ and X⁴ is —CN. In some embodiments, X¹ and X² are each H, and one of X³ and X⁴ is H and each of X³ and X⁴ is —CN. In some embodiments, X¹ and X² are each H, and one of X³ and X⁴ is C₁₋₆alkyl and the other of X³ and X⁴ is —CN. In some embodiments, X¹ and X² are each H, and one of X³ and X⁴ is halo and the other of X³ and X⁴ is —CN. In some embodiments, X¹ and X² are each H, and one of X³ and X⁴ is —CF₃ and the other of X³ and X⁴ is —CN. In some embodiments, one of X¹ and X² is H and the other of X¹ and X² is C₁₋₆alkyl, one X³ and X⁴ is halo and the other of X³ and X⁴ is —CN. In some embodiments, X¹ and X² are each H, one of X³ and X⁴ is H and the other of X³ and X⁴ is —SF₅.

In some embodiments, the phenyl ring bearing moieties X¹, X², X³, and X⁴ is selected from the group consisting of

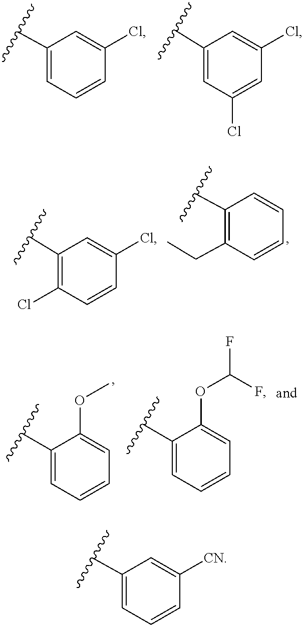

In some embodiments, the phenyl ring bearing moieties X¹, X², X³, and X⁴ is

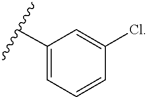 [SMILES: *c1cccc(Cl)c1]

In some embodiments, the phenyl ring bearing moieties X¹, X², X³, and X⁴ is selected from the group consisting of

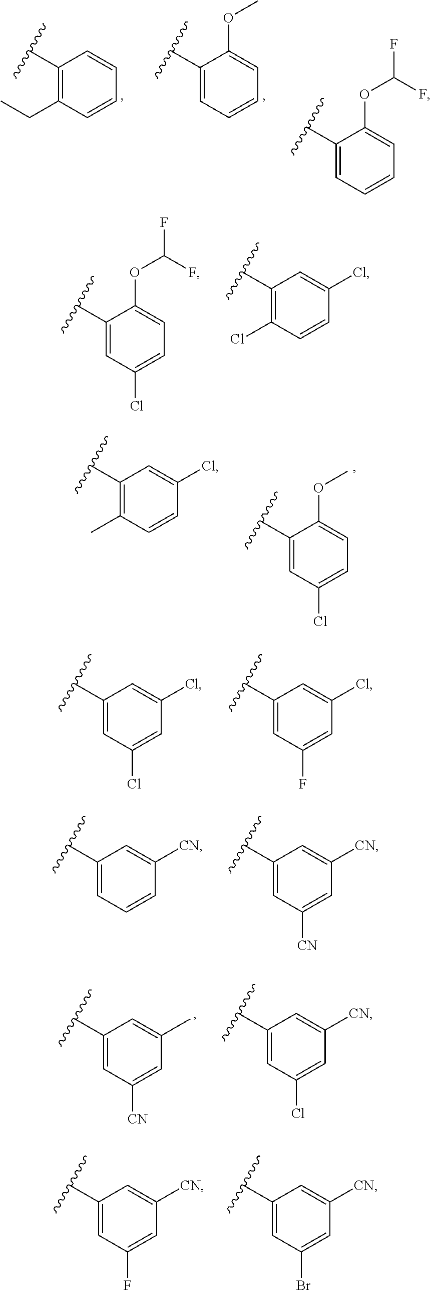

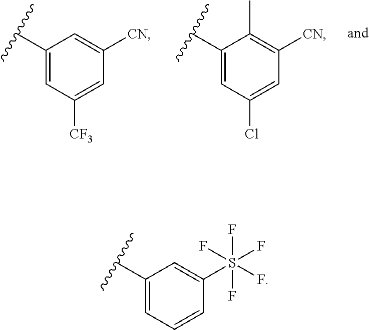 [SMILES: *c1cc(C#N)cc(C(F)(F)F)c1 ; *c1cc(Cl)cc(C#N)c1C ; *c1cccc(S(F)(F)(F)(F)F)c1]

In some embodiments, the phenyl ring bearing moieties X¹, X², X³, and X⁴ is selected from the group consisting of

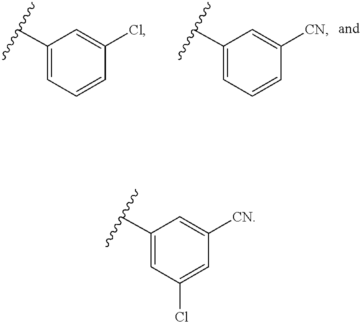 [SMILES: *c1cccc(Cl)c1 ; *c1cccc(C#N)c1 ; *c1cc(Cl)cc(C#N)c1]

In some embodiments, the phenyl ring bearing moieties X¹, X², X³, and X⁴ is

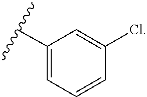 [SMILES: *c1cccc(Cl)c1]

In some embodiments, provided herein is a compound of formula (A), such as a compound of formula (I), (I-A1), (I-A2), (I-A3), (I-B1), (I-B2), (I-B3), (I-C1), (I-C2), (I-C3), (I-D1), (I-D2), (I-D3), (I-E1), (I-E2), (I-E3), (I-F1), (I-F2), (I-F3), (I-G1), (I-G2), (I-G3), (I-H-1), (I-H-2), (I-H-3), (II), (II-A1), (II-A2), (II-A3), (II-B1), (II-C1), (II-C2), (II-C3), (II-D1), (II-D2), (II-D3), (II-E1), (III), (III-A1), (III-A2), or (III-A3), or a stereoisomer or tautomer thereof, or a pharmaceutically acceptable salt of any of the foregoing, wherein Q is absent. In some embodiments, provided herein is a compound of formula (A), or any variation or embodiment thereof, or a stereoisomer or tautomer thereof, or a pharmaceutically acceptable salt of any of the foregoing, wherein the compound is of formula (B):

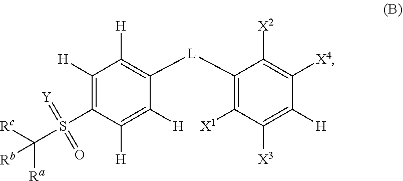

or a stereoisomer or tautomer thereof, or a pharmaceutically acceptable salt of any of the foregoing. In some variations, X¹, X², X³, X⁴, R^{a}, R^{b}, R^{c}, L and Y of formula (B) are as defined for formula (B) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (B), or a stereoisomer or tautomer thereof, or a pharmaceutically acceptable salt of any of the foregoing, wherein Y is O, wherein the compound is of formula (B-1):

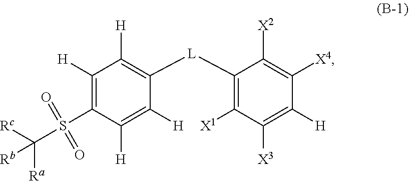

or a stereoisomer or tautomer thereof, or a pharmaceutically acceptable salt of any of the foregoing. In some variations, X¹, X², X³, X⁴, R^{a}, Rh, R^{c}, and L of formula (B1) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A), formula (B), or formula (B-1), or a stereoisomer or tautomer thereof, or a pharmaceutically acceptable salt of any of the foregoing, wherein R^{a}, R^{b}, and R^{c} are each independently H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl. In some embodiments, two of R^{a}, R^{b}, and R^{c} are independently H, and one of R^{a}, R^{b}, and R^{c} is C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl. In some embodiments, two of R^{a}, R^{b}, and R^{c} are independently H, and one of R^{a}, R^{b}, and R^{c} is,

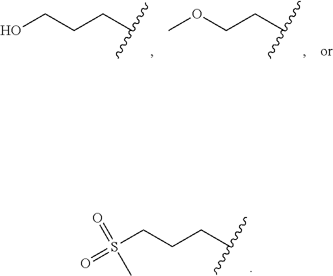

In some embodiments, provided herein is a compound of formula (A′), formula (B), or formula (B-1), or a stereoisomer or tautomer thereof, or a pharmaceutically acceptable salt of any of the foregoing, wherein R^{a}, R^{b}, and R^{c} are each independently H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl. In some embodiments, two of R^{a}, R^{b}, and R^{c} are independently H, and one of R^{a}, R^{b}, and R^{c} is C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl. In some embodiments, two of R^{a}, R^{b}, and R^{c} are independently H, and one of R^{a}, R^{b}, and R^{c} is

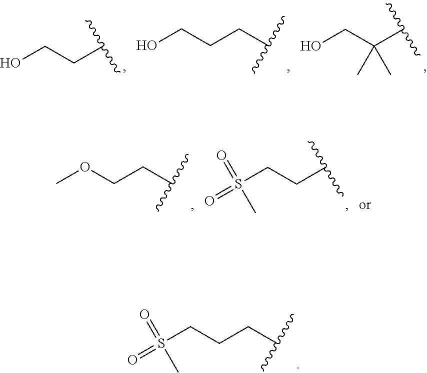

In some embodiments, provided herein is a compound of formula (A), formula (B), or formula (B-1), or a stereoisomer or tautomer thereof, or a pharmaceutically acceptable salt of any of the foregoing, wherein any two of R^{a}, R^{b}, and R^{c} are taken, together with the atoms to which they are attached, to form a C₃₋₆cycloalkyl or a 3-6 membered heterocyclyl, and the other of R^{a}, R^{b}, and R^{c} is H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl. In some embodiments, any two of R^{a}, R^{b}, and R^{c} are taken, together with the atoms to which they are attached, to form a 3-6 membered heterocyclyl, and the other of R^{a}, R^{b}, and R^{c} is H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl. In some embodiments, any two of R^{a}, R^{b}, and R^{c} are taken, together with the atoms to which they are attached, to form oxetanyl, and the other of R^{a}, R^{b}, and R^{c} is H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl. In some variations, the embodiments provided herein also apply to a compound of formula (A′) or a stereoisomer or tautomer thereof, or a pharmaceutically acceptable salt of any of the foregoing, or any variation or embodiment thereof.

In some embodiments, provided herein is a compound of formula (A), formula (B), or formula (B-1), or a stereoisomer or tautomer thereof, or a pharmaceutically acceptable salt of any of the foregoing, wherein R^{a}, R^{b}, and R^{c} are each H, wherein the compound is of formula (B-2):

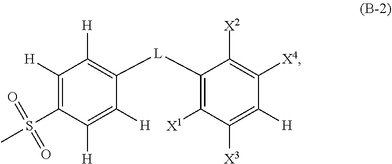

or a stereoisomer or tautomer thereof, or a pharmaceutically acceptable salt of any of the foregoing. In some variations, X¹, X², X³, X⁴, and L of formula (B-2) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A), formula (B), formula (B-1), or formula (B-2), or a stereoisomer or tautomer thereof, or a pharmaceutically acceptable salt of any of the foregoing, wherein one of X¹ and X² is H and the other of X¹ and X² is chloro, X³ is H, and X⁴ is H, wherein the compound is of formula (B-3):

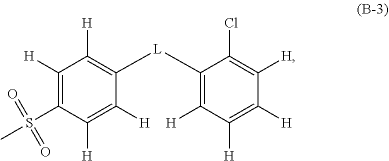

or a stereoisomer or tautomer thereof, or a pharmaceutically acceptable salt of any of the foregoing. In some variations, L of formula (B-3) is as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (B), or a stereoisomer or tautomer thereof, or a pharmaceutically acceptable salt of any of the foregoing, wherein Y is —N(C₁₋₆alkyl). In some embodiments, Y is —N(CH₃), wherein the compound is of formula (B-4):

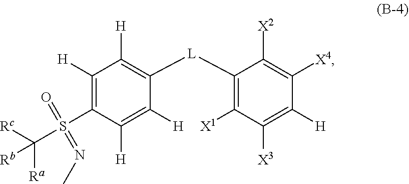

or a stereoisomer or tautomer thereof, or a pharmaceutically acceptable salt of any of the foregoing. In some variations, X¹, X², X³, X⁴, R^{a}, R^{b}, R^{c}, and L of formula (B-4) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A), formula (B), or formula (B-4), or a stereoisomer or tautomer thereof, or a pharmaceutically acceptable salt of any of the foregoing, wherein R^{a}, R^{b}, and R^{c} are each independently H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl. In some embodiments, two of R^{a}, R^{b}, and R^{c} are independently H, and one of R^{a}, R^{b}, and R^{c} is C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl. In some embodiments, two of R^{a}, R^{b}, and R^{c} are independently H, and one of R^{a}, R^{b}, and R^{c} is,

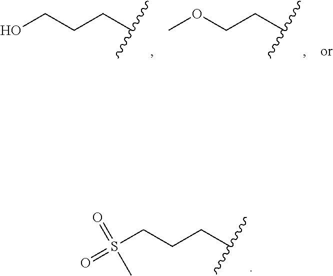

In some embodiments, provided herein is a compound of formula (A′), formula (B), or formula (B-4), or a stereoisomer or tautomer thereof, or a pharmaceutically acceptable salt of any of the foregoing, wherein R^{a}, R^{b}, and R^{c} are each independently H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl. In some embodiments, two of R^{a}, R^{b}, and R^{c} are independently H, and one of R^{a}, R^{b}, and R^{c} is C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl. In some embodiments, two of R^{a}, R^{b}, and R^{c} are independently H, and one of R^{a}, R^{b}, and R^{c} is,

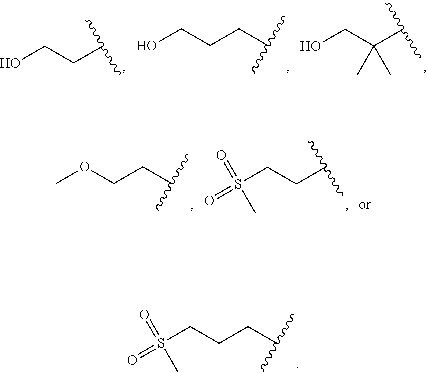

In some embodiments, provided herein is a compound of formula (A), formula (B), or formula (B-4), or a stereoisomer or tautomer thereof, or a pharmaceutically acceptable salt of any of the foregoing, wherein any two of R^{a}, R^{b}, and R^{c} are taken, together with the atoms to which they are attached, to form a C₃₋₆cycloalkyl or a 3-6 membered heterocyclyl, and the other of R^{a}, R^{b}, and R^{c} is H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl. In some embodiments, any two of R^{a}, R^{b}, and R^{c} are taken, together with the atoms to which they are attached, to form a 3-6 membered heterocyclyl, and the other of R^{a}, R^{b}, and R^{c} is H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl. In some embodiments, any two of R^{a}, R^{b}, and R^{c} are taken, together with the atoms to which they are attached, to form oxetanyl, and the other of R^{a}, R^{b}, and R^{c} is H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl.

In some embodiments, provided herein is a compound of formula (A′), formula (B), or formula (B-4), or a stereoisomer or tautomer thereof, or a pharmaceutically acceptable salt of any of the foregoing, wherein any two of R^{a}, R^{b}, and R^{c} are taken, together with the atoms to which they are attached, to form a C₃₋₆cycloalkyl or a 3-6 membered heterocyclyl, and the other of R^{a}, R^{b}, and R^{c} is H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl. In some embodiments, any two of R^{a}, R^{b}, and R^{c} are taken, together with the atoms to which they are attached, to form a 3-6 membered heterocyclyl, and the other of R^{a}, R^{b}, and R^{c} is H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl. In some embodiments, any two of R^{a}, R^{b}, and R^{c} are taken, together with the atoms to which they are attached, to form oxetanyl, and the other of R^{a}, R^{b}, and R^{c} is H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl. In some embodiments, any two of R^{a}, R^{b}, and R^{c} are taken, together with the atoms to which they are attached, to form azetidinyl, and the other of R^{a}, R^{b}, and R^{c} is H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl.

In some embodiments, provided herein is a compound of formula (A), formula (B), or formula (B-4), or a stereoisomer or tautomer thereof, or a pharmaceutically acceptable salt of any of the foregoing, wherein R^{a}, R^{b}, and R^{c} are each H, wherein the compound is of formula (B-5):

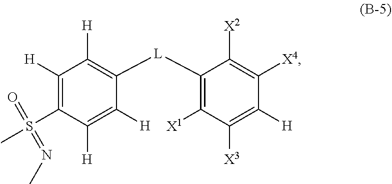

or a stereoisomer or tautomer thereof, or a pharmaceutically acceptable salt of any of the foregoing. In some variations, X¹, X², X³, X⁴, and L of formula (B-5) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A), formula (B), formula (B-4), or formula (B-5), or a stereoisomer or tautomer thereof, or a pharmaceutically acceptable salt of any of the foregoing, wherein one of X¹ and X² is H and the other of X¹ and X² is chloro, X³ is H, and X⁴ is H, wherein the compound is of formula (B-6):

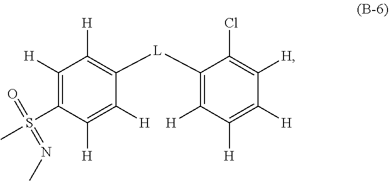

or a stereoisomer or tautomer thereof, or a pharmaceutically acceptable salt of any of the foregoing. In some variations, L of formula (B-6) is as defined for is a compound of formula (A′), or a stereoisomer or tautomer thereof, or a pharmaceutically acceptable salt of any of the foregoing, or any variation or embodiment thereof.

In some embodiments, provided herein is a compound of formula (A), such as a compound of formula (I), (I-A1), (I-A2), (I-A3), (I-B1), (I-B2), (I-B3), (I-C1), (I-C2), (I-C3), (I-D1), (I-D2), (I-D3), (I-E1), (I-E2), (I-E3), (I-F1), (I-F2), (I-F3), (I-G1), (I-G2), (I-G3), (I-H-1), (I-H-2), (I-H-3), (II), (II-A1), (II-A2), (II-A3), (II-B1), (II-C1), (II-C2), (II-C3), (II-D1), (II-D2), (II-D3), (II-E1), (III), (III-A1), (III-A2), or (III-A3), or a stereoisomer or tautomer thereof, or a pharmaceutically acceptable salt of any of the foregoing, wherein Q is —N(C₁₋₆alkyl). In some embodiments, Q is —N(CH₃). In some embodiments, provided herein is a compound of formula (A), or any variation or embodiment thereof, or a stereoisomer or tautomer thereof, or a pharmaceutically acceptable salt of any of the foregoing, wherein the compound is of formula (C):

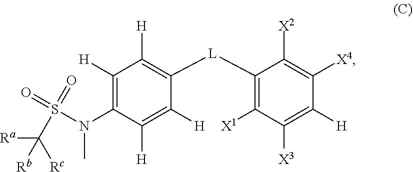

or a stereoisomer or tautomer thereof, or a pharmaceutically acceptable salt of any of the foregoing. In some variations, X¹, X², X³, X⁴, R^{a}, R^{b}, R^{c}, and L of formula (C) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A) or formula (C), or a stereoisomer or tautomer thereof, or a pharmaceutically acceptable salt of any of the foregoing, wherein R^{a}, R^{b}, and R^{c} are each independently H or C₁₋₆alkyl, wherein the C₁₋₆ alkyl of R^{a}, R^{b}, or R^{c} is independently optionally substituted with one or more —OH, C₁₋₆alkoxy, or —S(O)₂-C₁₋₆alkyl. In some embodiments, two of R^{a}, R^{b}, and R^{c} are independently H, and one of R^{a}, R^{b}, and R^{c} is C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl. In some embodiments, two of R^{a}, R^{b}, and R^{c} are independently H, and one of R^{a}, R^{b}, and R^{c} is

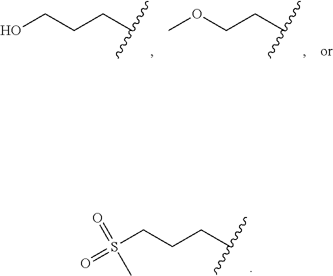

In some embodiments, provided herein is a compound of formula (A′) or formula (C), or a stereoisomer or tautomer thereof, or a pharmaceutically acceptable salt of any of the foregoing, wherein R^{a}, R^{b}, and R^{c} are each independently H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl. In some embodiments, two of R^{a}, R^{b}, and R^{c} are independently H, and one of R^{a}, R^{b}, and R^{c} is C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl. In some embodiments, two of R^{a}, R^{b}, and R^{c} are independently H, and one of R^{a}, R^{b}, and R^{c} is

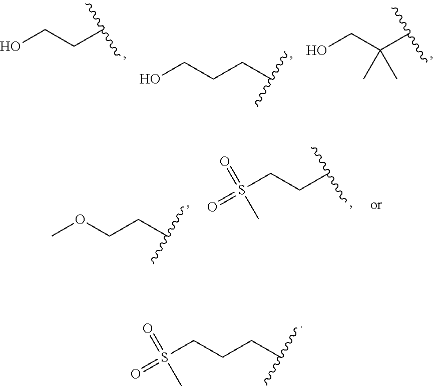

In some embodiments, provided herein is a compound of formula (A) or formula (C), or a stereoisomer or tautomer thereof, or a pharmaceutically acceptable salt of any of the foregoing, wherein any two of R^{a}, R^{b}, and R^{c} are taken, together with the atoms to which they are attached, to form a C₃₋₆cycloalkyl or a 3-6 membered heterocyclyl, and the other of R^{a}, R^{b}, and R^{c} is H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl. In some embodiments, any two of R^{a}, R^{b}, and R^{c} are taken, together with the atoms to which they are attached, to form a 3-6 membered heterocyclyl, and the other of R^{a}, R^{b}, and R^{c} is H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl. In some embodiments, any two of R^{a}, R^{b}, and R^{c} are taken, together with the atoms to which they are attached, to form oxetanyl, and the other of R^{a}, R^{b}, and R^{c} is H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl. In some variations, the embodiments provided herein also apply to a compound of formula (A′) or a stereoisomer or tautomer thereof, or a pharmaceutically acceptable salt of any of the foregoing, or any variation or embodiment thereof.

In some embodiments, provided herein is a compound of formula (A) or formula (C), or a stereoisomer or tautomer thereof, or a pharmaceutically acceptable salt of any of the foregoing, wherein R^{a}, R^{b}, and R^{c} are each H, wherein the compound is of formula (C-1):

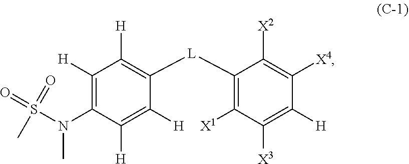

or a stereoisomer or tautomer thereof, or a pharmaceutically acceptable salt of any of the foregoing. In some variations, X¹, X², X³, X⁴, and L of formula (C-1) are as defined for a compound of formula (A′), or a stereoisomer or tautomer thereof, or a pharmaceutically acceptable salt of any of the foregoing, or any variation or embodiment thereof.

In some embodiments, provided herein is a compound of formula (A), formula (C), or formula (C-1), or a stereoisomer or tautomer thereof, or a pharmaceutically acceptable salt of any of the foregoing, wherein one of X¹ and X² is H and the other of X¹ and X² is chloro, X³ is H, and X⁴ is H, wherein the compound is of formula (C-2):

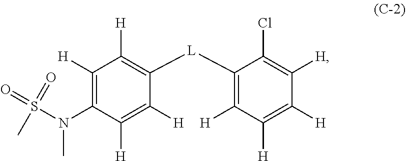

or a stereoisomer or tautomer thereof, or a pharmaceutically acceptable salt of any of the foregoing. In some variations, L of formula (C-2) is as defined for a compound of formula (A′), or a stereoisomer or tautomer thereof, or a pharmaceutically acceptable salt of any of the foregoing, or any variation or embodiment thereof.

In some embodiments, provided herein is a compound of formula (A), such as a compound of formula (B), (B-1), (B-2), (B-3), (B-4), (B-5), (B-6), (C), (C-1), or (C-2), or a stereoisomer or tautomer thereof, or a pharmaceutically acceptable salt of any of the foregoing, wherein L is

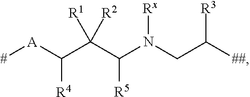

or any embodiment or variation thereof, as described elsewhere herein, including, for example, as in a compound of formula (I), (I-A1), (I-A2), (I-A3), (I-B1), (I-B2), (I-B3), (I-C1), (I-C2), (I-C3), (I-D1), (I-D2), (I-D3), (I-E1), (I-E2), (I-E3), (I-F1), (I-F2), (I-F3), (I-G1), (I-G2), (I-G3), (I-H-1), (I-H-2), (I-H-3), (II), (II-A1), (II-A2), (II-A3), (II-B1), (II-C1), (II-C2), (II-C3), (II-D1), (II-D2), (II-D3), (II-E1), (III), (III-A1), (III-A2), or (III-A3), or a stereoisomer or tautomer thereof, or a pharmaceutically acceptable salt of any of the foregoing.

In some embodiments, provided herein is a compound of formula (A′), such as a compound of formula (B), (B-1), (B-2), (B-3), (B-4), (B-5), (B-6), (C), (C-1), or (C-2), or a stereoisomer or tautomer thereof, or a pharmaceutically acceptable salt of any of the foregoing, wherein L is

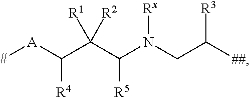

or any embodiment or variation thereof, as described elsewhere herein, including, for example, as in a compound of formula (I), (I-A1), (I-A2), (I-A3), (I-B1), (I-B2), (I-B3), (I-C1), (I-C2), (I-C3), (I-D1), (I-D2), (I-D3), (I-E1), (I-E2), (I-E3), (I-F1), (I-F2), (I-F3), (I-G1), (I-G2), (I-G3), (I-H-1), (I-H-2), (I-H-3), (II), (II-A1), (II-A2), (II-A3), (II-B1), (II-C1), (II-C2), (II-C3), (II-D1), (II-D2), (II-D3), (II-E1), (III), (III-A1), (III-A2), or (III-A3), or a stereoisomer or tautomer thereof, or a pharmaceutically acceptable salt of any of the foregoing.

In some embodiments, provided herein is a compound of formula (A), such as a compound of formula (B), (B-1), (B-2), (B-3), (B-4), (B-5), (B-6), (C), (C-1), or (C-2), or a stereoisomer or tatomer thereof, or a pharmaceutically acceptable salt of any of the foregoing, wherein L is

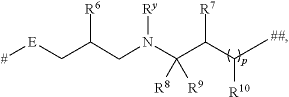

or any embodiment or variation thereof, as described elsewhere herein, including, for example, as in a compound of formula (I), (I-A1), (I-A2), (I-A3), (I-B1), (I-B2), (I-B3), (I-C1), (I-C2), (I-C3), (I-D1), (I-D2), (I-D3), (I-E1), (I-E2), (I-E3), (I-F1), (I-F2), (I-F3), (I-G1), (I-G2), (I-G3), (I-H-1), (I-H-2), (I-H-3), (II), (II-A1), (II-A2), (II-A3), (II-B1), (II-C1), (II-C2), (II-C3), (II-D1), (II-D2), (II-D3), (II-E1), (III), (III-A1), (III-A2), or (III-A3), or a stereoisomer or tautomer thereof, or a pharmaceutically acceptable salt of any of the foregoing.

In some embodiments, provided herein is a compound of formula (A′), such as a compound of formula (B), (B-1), (B-2), (B-3), (B-4), (B-5), (B-6), (C), (C-1), or (C-2), or a stereoisomer or tatomer thereof, or a pharmaceutically acceptable salt of any of the foregoing, wherein L is

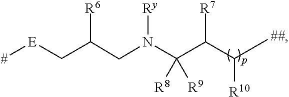

or any embodiment or variation thereof, as described elsewhere herein, including, for example, as in a compound of formula (I), (I-A1), (I-A2), (I-A3), (I-B1), (I-B2), (I-B3), (I-C1), (I-C2), (I-C3), (I-D1), (I-D2), (I-D3), (I-E1), (I-E2), (I-E3), (I-F1), (I-F2), (I-F3), (I-G1), (I-G2), (I-G3), (I-H-1), (I-H-2), (I-H-3), (II), (II-A1), (II-A2), (II-A3), (II-B1), (II-C1), (II-C2), (II-C3), (II-D1), (II-D2), (II-D3), (II-E1), (III), (III-A1), (III-A2), or (III-A3), or a stereoisomer or tautomer thereof, or a pharmaceutically acceptable salt of any of the foregoing.

In some embodiments, provided herein is a compound of formula (A), such as a compound of formula (B), (B-1), (B-2), (B-3), (B-4), (B-5), (B-6), (C), (C-1), or (C-2), or a stereoisomer or tautomer thereof, or a pharmaceutically acceptable salt of any of the foregoing, wherein L is

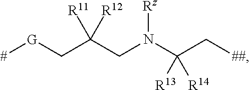

or any embodiment or variation thereof, as described elsewhere herein, including, for example, as in a compound of formula (I), (I-A1), (I-A2), (I-A3), (I-B1), (I-B2), (I-B3), (I-C1), (I-C2), (I-C3), (I-D1), (I-D2), (I-D3), (I-E1), (I-E2), (I-E3), (I-F1), (I-F2), (I-F3), (I-G1), (I-G2), (I-G3), (I-H-1), (I-H-2), (I-H-3), (II), (II-A1), (II-A2), (II-A3), (II-B1), (II-C1), (II-C2), (II-C3), (II-D1), (II-D2), (II-D3), (II-E1), (III), (III-A1), (III-A2), or (III-A3), or a stereoisomer or tautomer thereof, or a pharmaceutically acceptable salt of any of the foregoing.

In some embodiments, provided herein is a compound of formula (A′), such as a compound of formula (B), (B-1), (B-2), (B-3), (B-4), (B-5), (B-6), (C), (C-1), or (C-2), or a stereoisomer or tautomer thereof, or a pharmaceutically acceptable salt of any of the foregoing, wherein L is

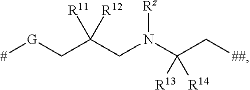

or any embodiment or variation thereof, as described elsewhere herein, including, for example, as in a compound of formula (I), (I-A1), (I-A2), (I-A3), (I-B1), (I-B2), (I-B3), (I-C1), (I-C2), (I-C3), (I-D1), (I-D2), (I-D3), (I-E1), (I-E2), (I-E3), (I-F1), (I-F2), (I-F3), (I-G1), (I-G2), (I-G3), (I-H-1), (I-H-2), (I-H-3), (II), (II-A1), (II-A2), (II-A3), (II-B1), (II-C1), (II-C2), (II-C3), (II-D1), (II-D2), (II-D3), (II-E1), (III), (III-A1), (III-A2), or (III-A3), or a stereoisomer or tautomer thereof, or a pharmaceutically acceptable salt of any of the foregoing.

In some embodiments, provided herein is a compound of formula (A), or formula (A′), or a pharmaceutically acceptable salt of any of the foregoing, wherein L is selected from the group consisting of,

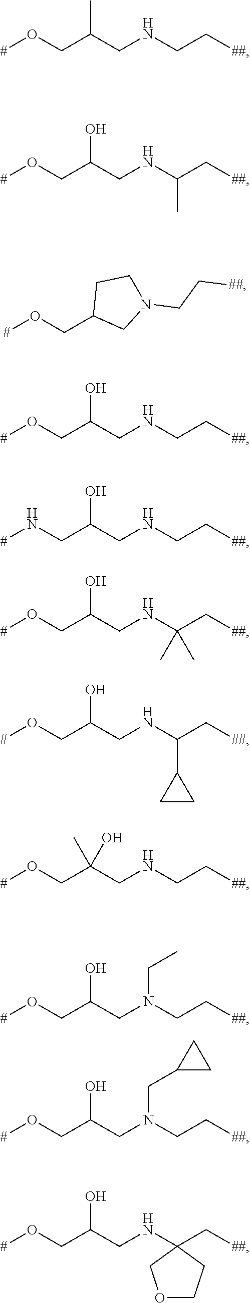

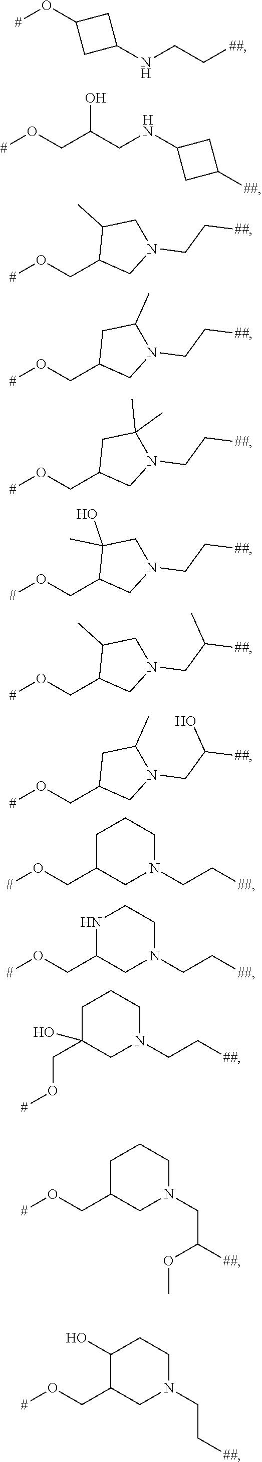

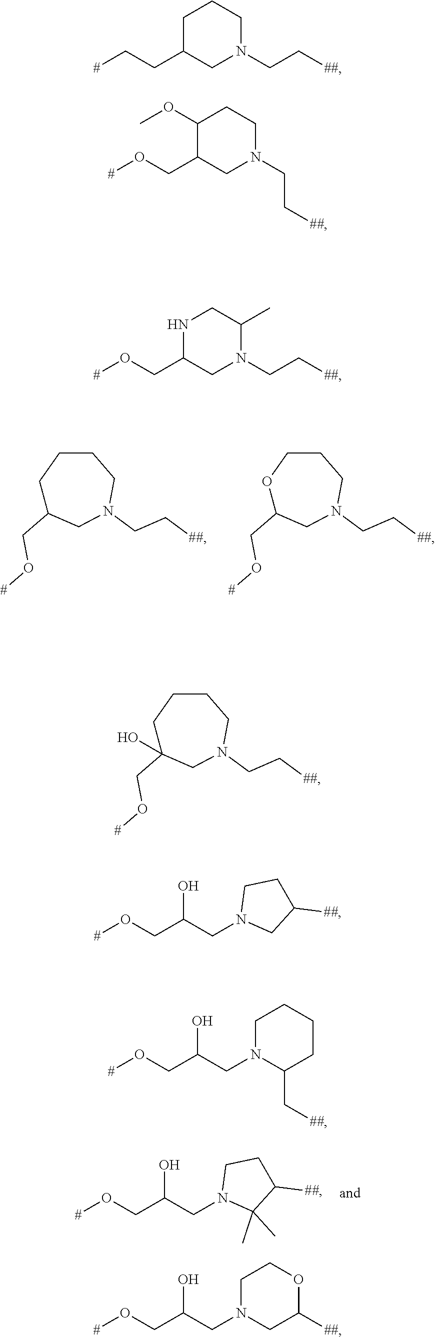

wherein #denotes the point of attachment to the phenyl ring bearing moiety Q, and ##denotes the point of attachment to the phenyl ring bearing moieties X¹-X⁴.

In some embodiments, provided herein is a compound of formula (A), or formula (A′), or a pharmaceutically acceptable salt of any of the foregoing, wherein L and the phenyl ring bearing moieties X¹-X⁴ together form a structure selected from the group consisting of

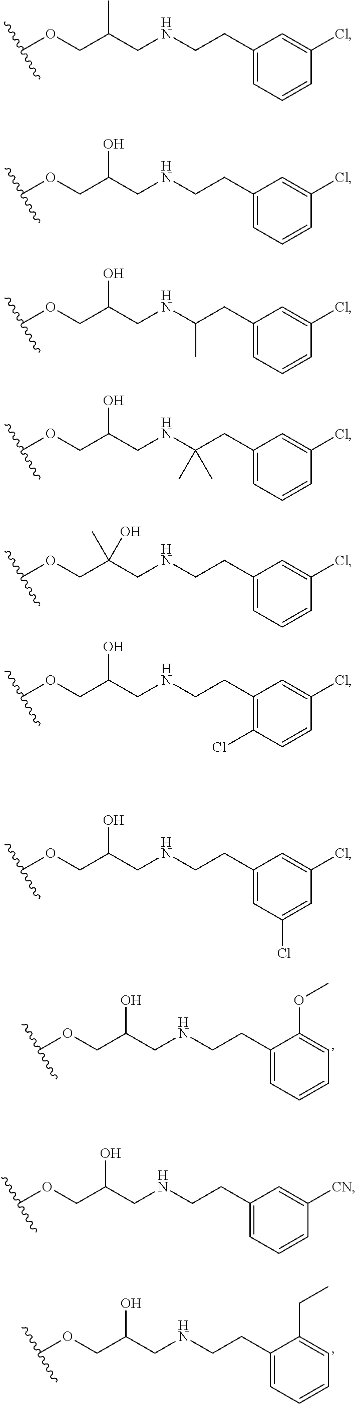

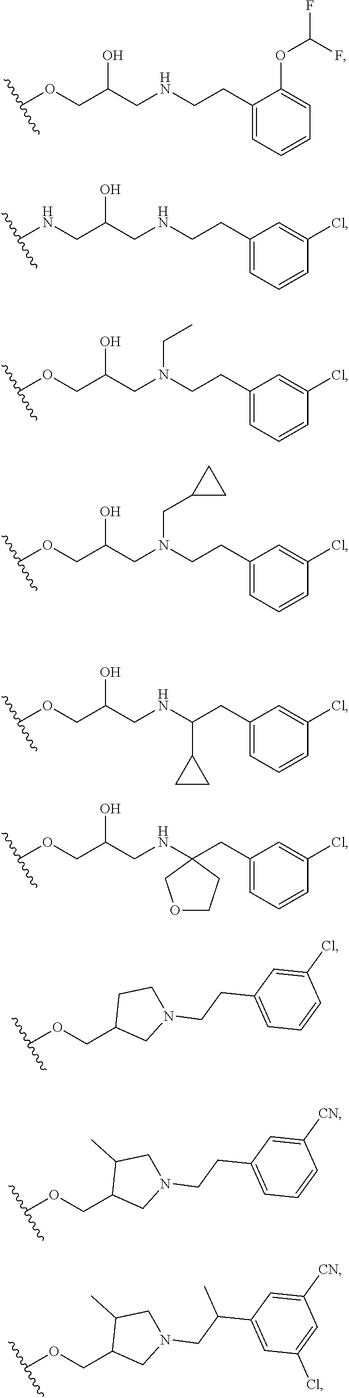

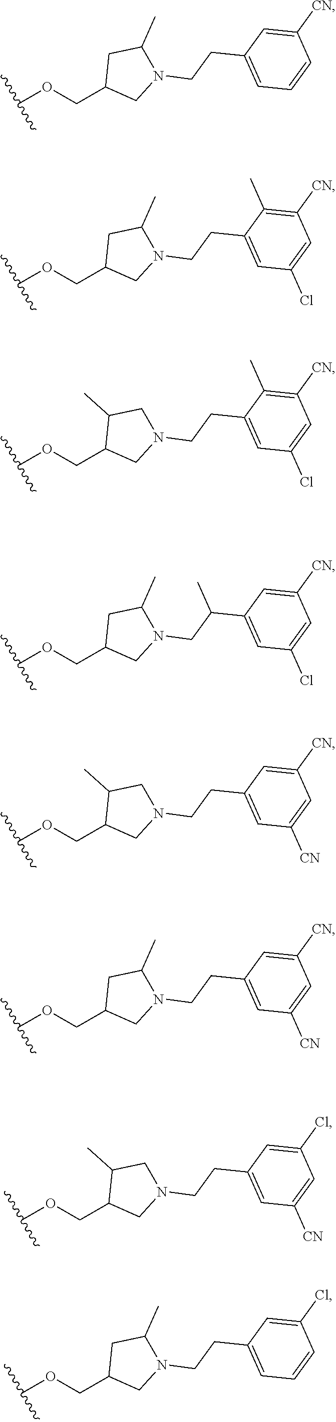

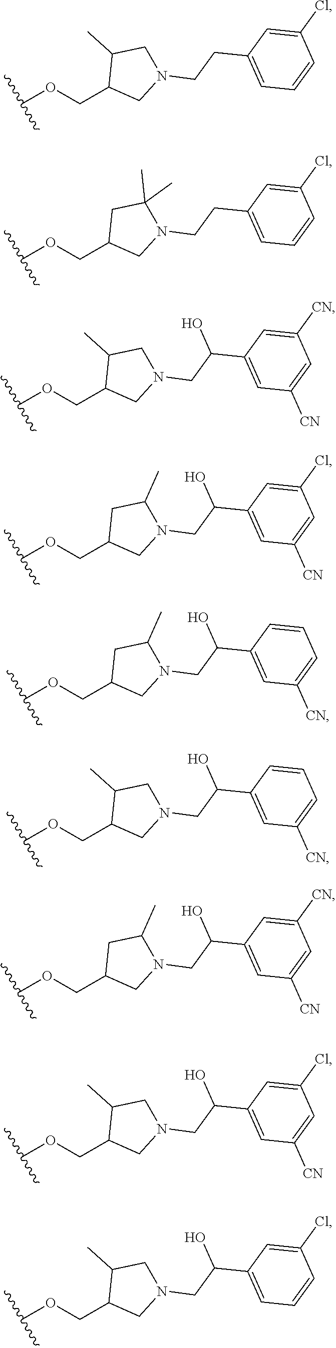

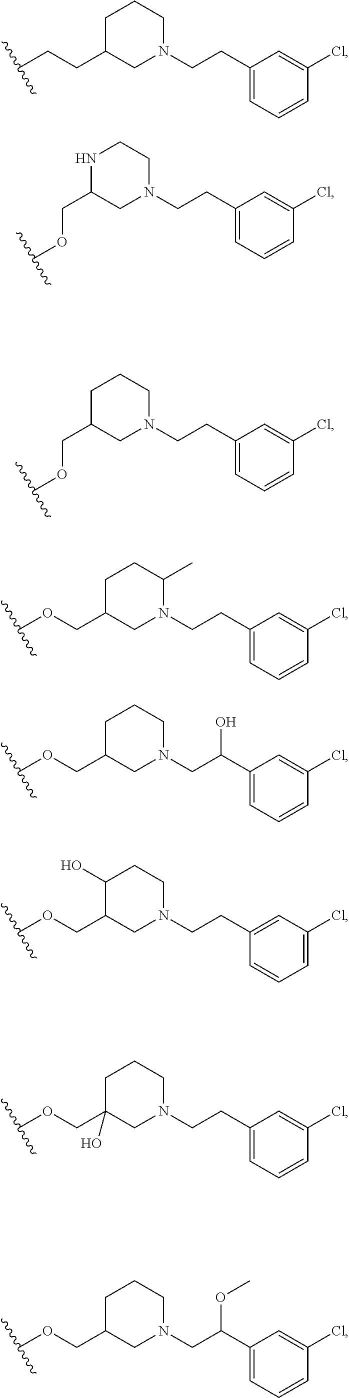

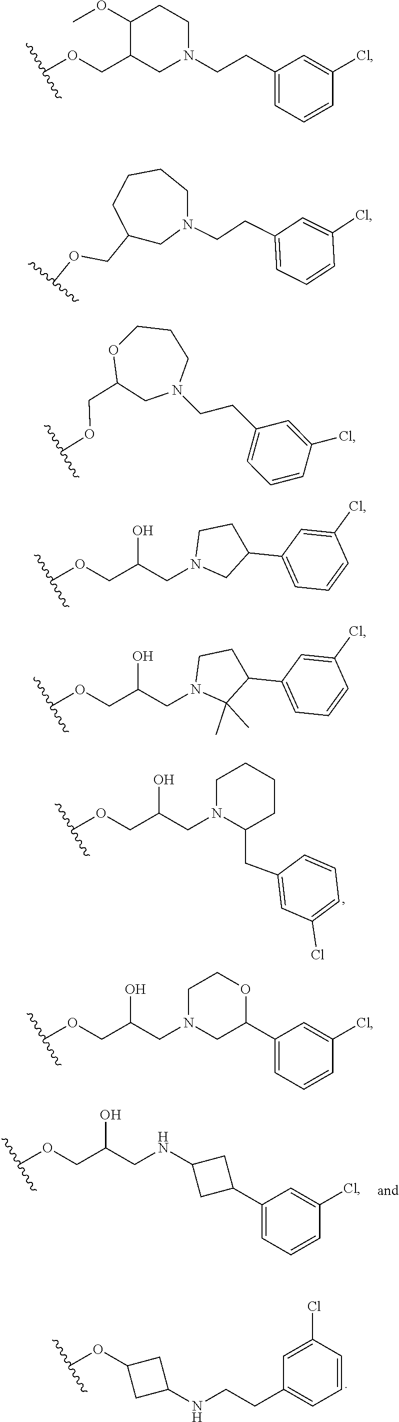

In some embodiments, provided herein is a compound of formula (A), or formula (A′), or a pharmaceutically acceptable salt of any of the foregoing, wherein the phenyl ring bearing Q is selected from the group consisting of

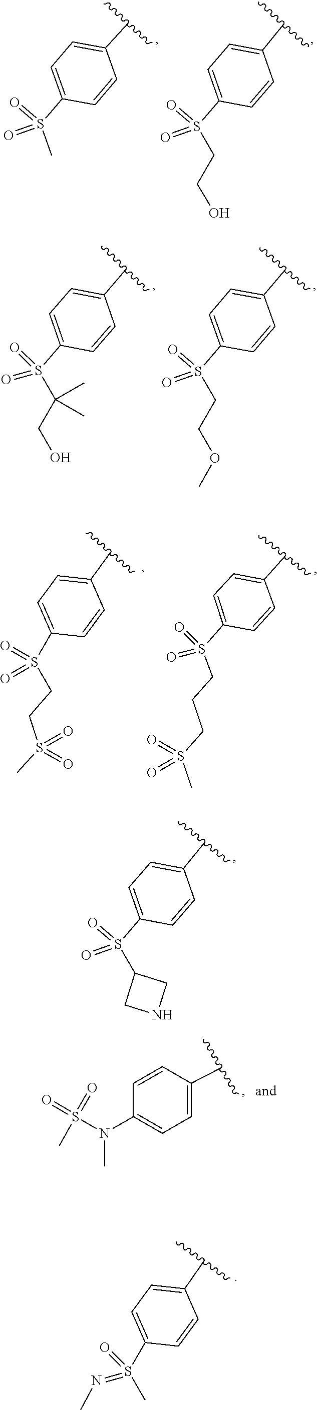

In some embodiments, provided herein is a compound of formula (A′), or a pharmaceutically acceptable salt of any of the foregoing, wherein L and the phenyl ring bearing moieties Q together form a structure selected from the group consisting of

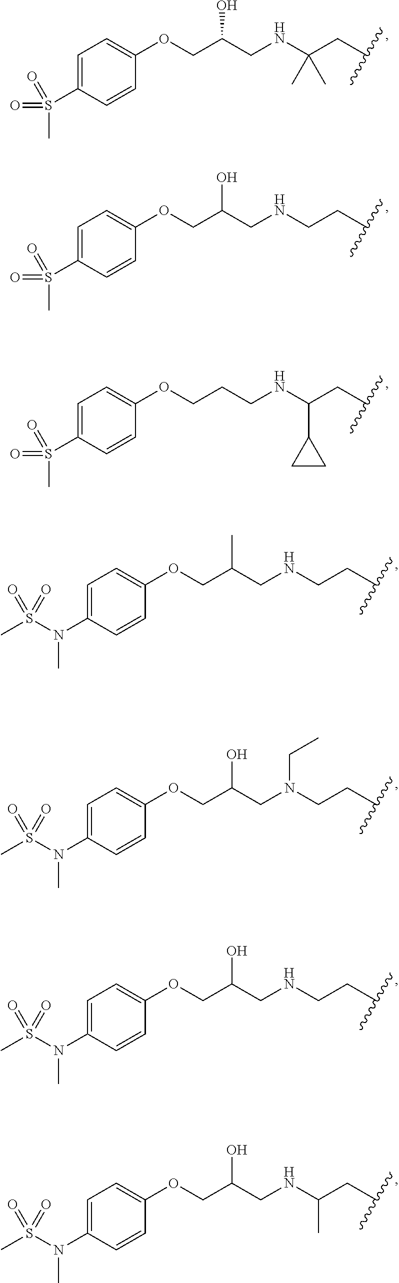

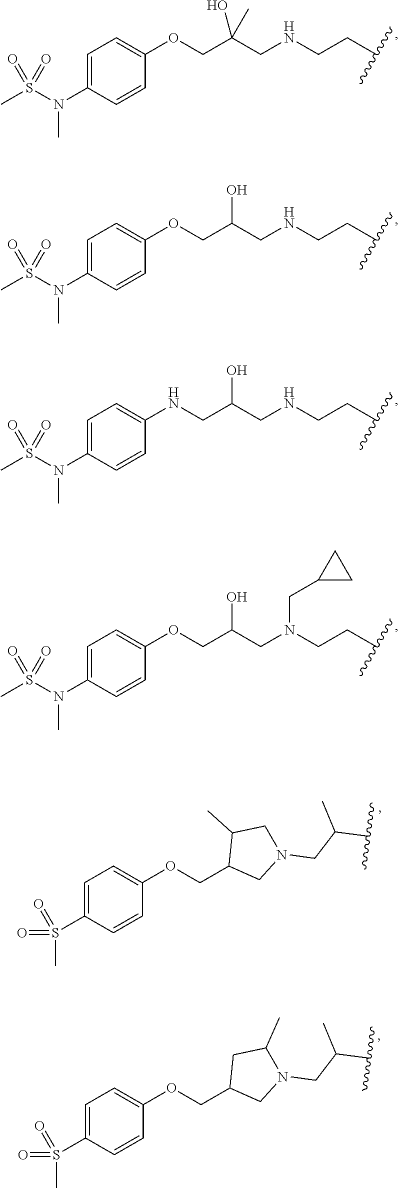 [SMILES: *CCNCC(C)(O)COc1ccc(N(C)S(C)(=O)=O)cc1 ; *CCNCC(O)COc1ccc(N(C)S(C)(=O)=O)cc1 ; *CCNCC(O)CNc1ccc(N(C)S(C)(=O)=O)cc1 ; *CCN(CC(O)COc1ccc(N(C)S(C)(=O)=O)cc1)CC1CC1 ; *C(C)CN1CC(C)C(COc2ccc(S(C)(=O)=O)cc2)C1 ; *C(C)CN1CC(COc2ccc(S(C)(=O)=O)cc2)CC1C]

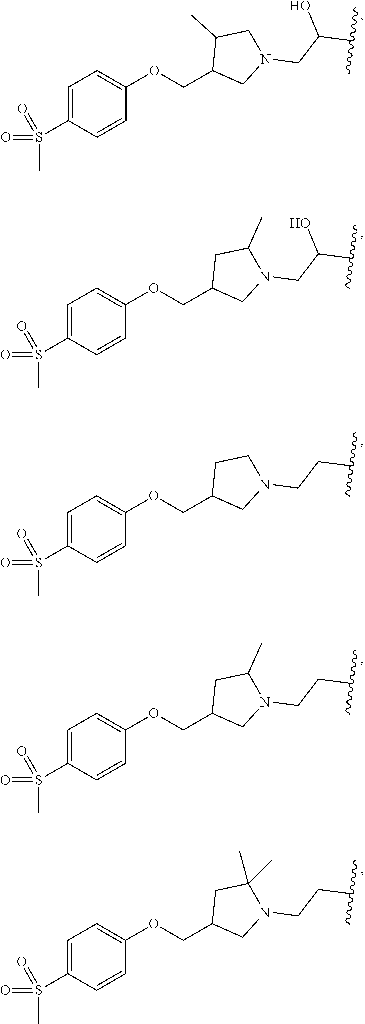 [SMILES: *C(O)CN1CC(C)C(COc2ccc(S(C)(=O)=O)cc2)C1 ; *C(O)CN1CC(COc2ccc(S(C)(=O)=O)cc2)CC1C ; *CCN1CCC(COc2ccc(S(C)(=O)=O)cc2)C1 ; *CCN1CC(COc2ccc(S(C)(=O)=O)cc2)CC1C ; *CCN1CC(COc2ccc(S(C)(=O)=O)cc2)CC1(C)C]

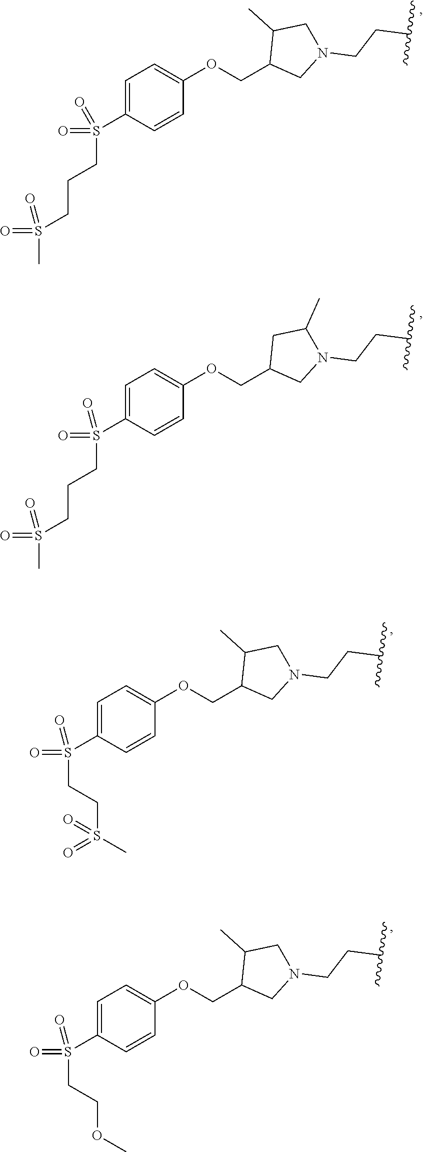 [SMILES: *CCN1CC(C)C(COc2ccc(S(=O)(=O)CCCS(C)(=O)=O)cc2)C1 ; *CCN1CC(COc2ccc(S(=O)(=O)CCCS(C)(=O)=O)cc2)CC1C ; *CCN1CC(C)C(COc2ccc(S(=O)(=O)CCS(C)(=O)=O)cc2)C1 ; *CCN1CC(C)C(COc2ccc(S(=O)(=O)CCOC)cc2)C1]

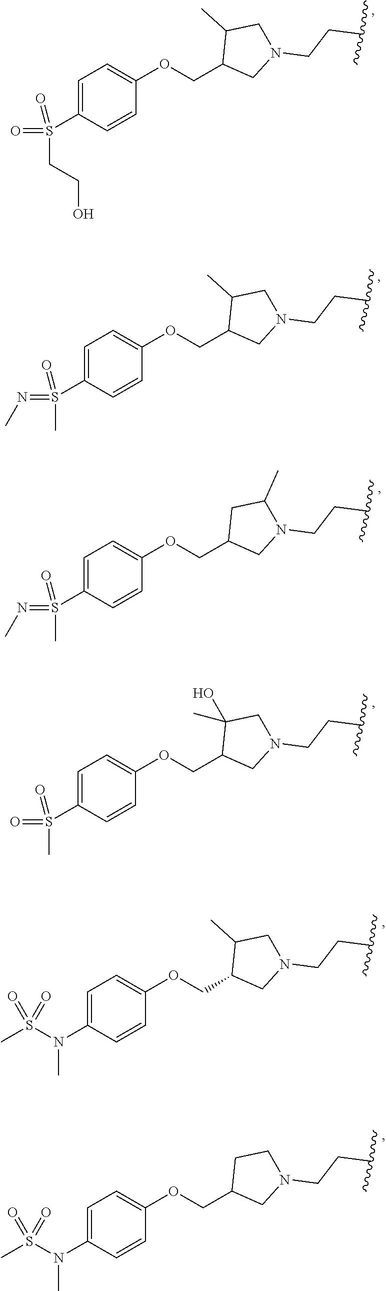 [SMILES: *CCN1CC(C)C(COc2ccc(S(=O)(=O)CCO)cc2)C1 ; *CCN1CC(C)C(COc2ccc(S(C)(=O)=NC)cc2)C1 ; *CCN1CC(COc2ccc(S(C)(=O)=NC)cc2)CC1C ; *CCN1CC(COc2ccc(S(C)(=O)=O)cc2)C(C)(O)C1 ; *CCN1CC(C)[C@@H](COc2ccc(N(C)S(C)(=O)=O)cc2)C1 ; *CCN1CCC(COc2ccc(N(C)S(C)(=O)=O)cc2)C1]

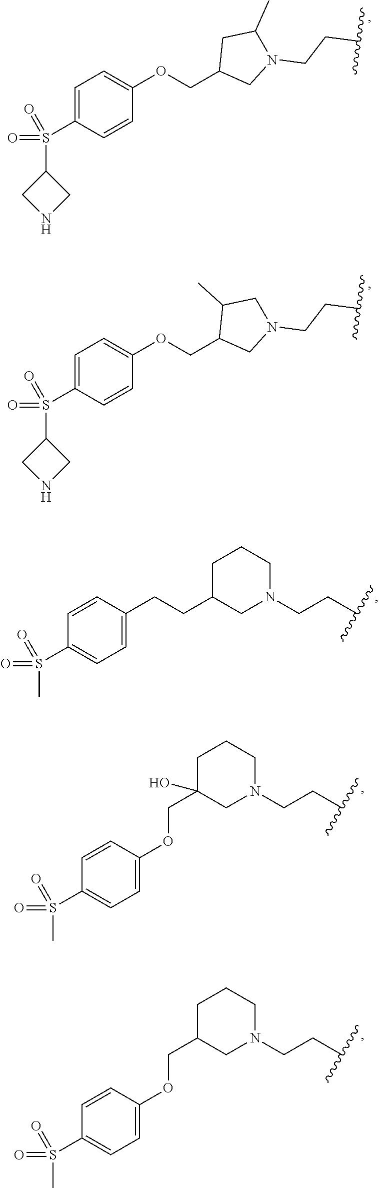 [SMILES: *CCN1CC(COc2ccc(S(=O)(=O)C3CNC3)cc2)CC1C ; *CCN1CC(C)C(COc2ccc(S(=O)(=O)C3CNC3)cc2)C1 ; *CCN1CCCC(CCc2ccc(S(C)(=O)=O)cc2)C1 ; *CCN1CCCC(O)(COc2ccc(S(C)(=O)=O)cc2)C1 ; *CCN1CCCC(COc2ccc(S(C)(=O)=O)cc2)C1]

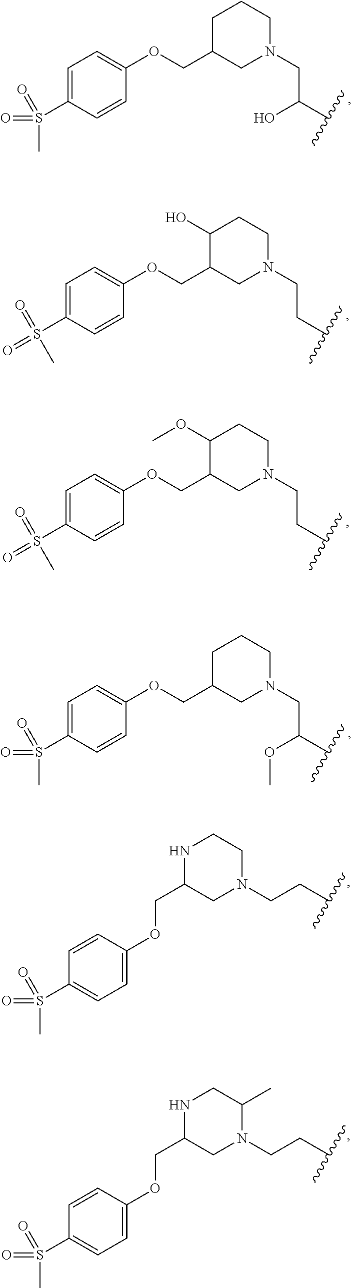 [SMILES: *C(O)CN1CCCC(COc2ccc(S(C)(=O)=O)cc2)C1 ; *CCN1CCC(O)C(COc2ccc(S(C)(=O)=O)cc2)C1 ; *CCN1CCC(OC)C(COc2ccc(S(C)(=O)=O)cc2)C1 ; *C(CN1CCCC(COc2ccc(S(C)(=O)=O)cc2)C1)OC ; *CCN1CCNC(COc2ccc(S(C)(=O)=O)cc2)C1 ; *CCN1CC(COc2ccc(S(C)(=O)=O)cc2)NCC1C]

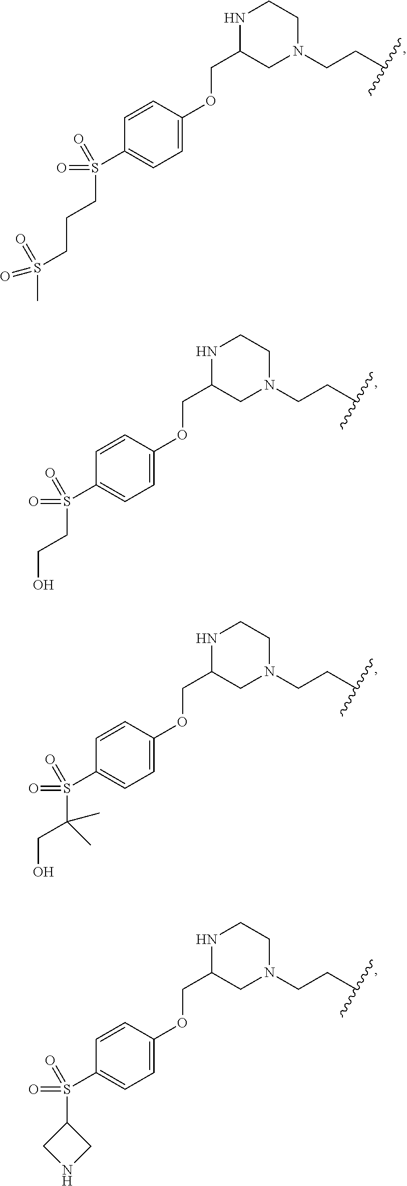 [SMILES: *CCN1CCNC(COc2ccc(S(=O)(=O)CCCS(C)(=O)=O)cc2)C1 ; *CCN1CCNC(COc2ccc(S(=O)(=O)CCO)cc2)C1 ; *CCN1CCNC(COc2ccc(S(=O)(=O)C(C)(C)CO)cc2)C1 ; *CCN1CCNC(COc2ccc(S(=O)(=O)C3CNC3)cc2)C1]

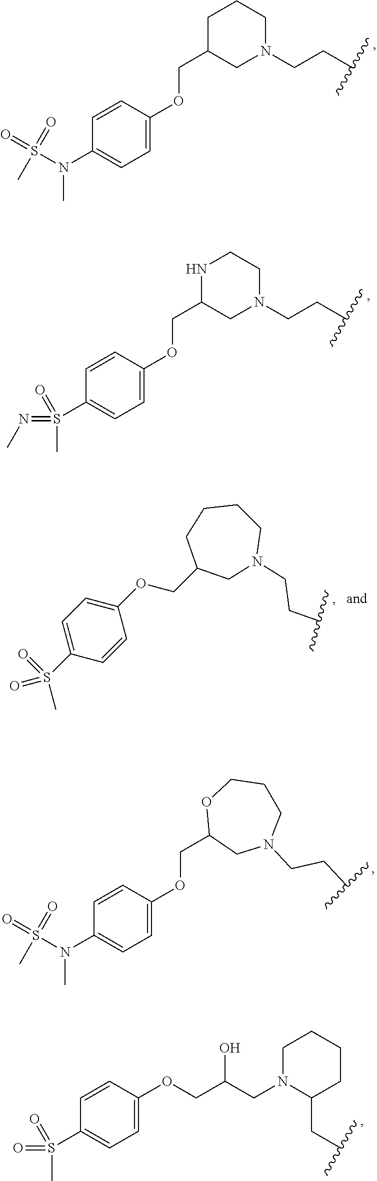 [SMILES: *CCN1CCCC(COc2ccc(N(C)S(C)(=O)=O)cc2)C1 ; *CCN1CCNC(COc2ccc(S(C)(=O)=NC)cc2)C1 ; *CCN1CCCCC(COc2ccc(S(C)(=O)=O)cc2)C1 ; *CCN1CCCOC(COc2ccc(N(C)S(C)(=O)=O)cc2)C1 ; *CC1CCCCN1CC(O)COc1ccc(S(C)(=O)=O)cc1]

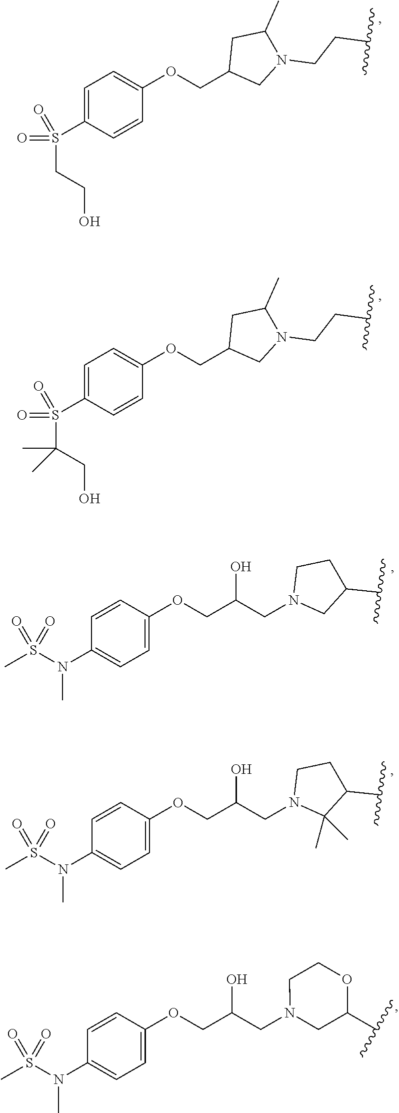 [SMILES: *CCN1CC(COc2ccc(S(=O)(=O)CCO)cc2)CC1C ; *CCN1CC(COc2ccc(S(=O)(=O)C(C)(C)CO)cc2)CC1C ; *C1CCN(CC(O)COc2ccc(N(C)S(C)(=O)=O)cc2)C1 ; *C1CCN(CC(O)COc2ccc(N(C)S(C)(=O)=O)cc2)C1(C)C ; *C1CN(CC(O)COc2ccc(N(C)S(C)(=O)=O)cc2)CCO1]

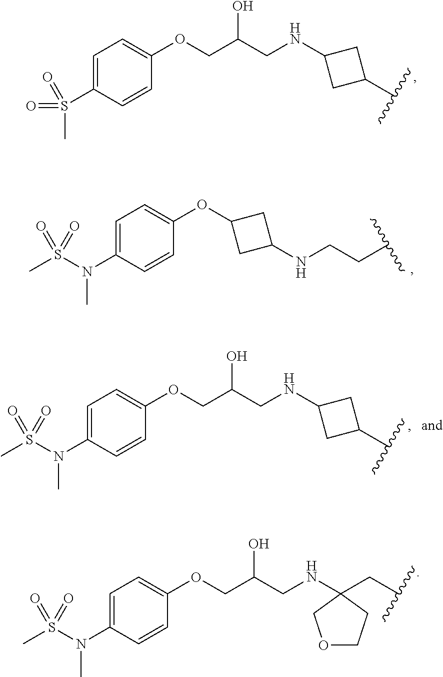 [SMILES: *C1CC(NCC(O)COc2ccc(S(C)(=O)=O)cc2)C1 ; *CCNC1CC(Oc2ccc(N(C)S(C)(=O)=O)cc2)C1 ; *C1CC(NCC(O)COc2ccc(N(C)S(C)(=O)=O)cc2)C1 ; *CC1(NCC(O)COc2ccc(N(C)S(C)(=O)=O)cc2)CCOC1]

It is to be understood that any variation or embodiment of X¹, X², X³, X⁴, R¹, R², R³, R⁴, R⁵, R⁶, R⁷, R⁸, R⁹, R¹⁰, R¹¹, R¹², R¹³, R¹⁴, R^{a}, R^{b}, R^{c}, R^{g}, R^{x}, R^{y}, R^{z}, n, p, A, E, G, L, Q, and Y provided herein can be combined with every other variation or embodiment of X¹, X², X³, X⁴, R¹, R², R³, R⁴, R⁵, R⁶, R⁷, R⁸, R^{g}, R¹⁰, R¹¹, R¹², R¹³, R¹⁴, R^{a}, R^{b}, R^{c}, R^{g}, R^{x}, R^{y}, R^{z}, n, p, A, E, G, L, Q, and Y, the same as if each and every combination had been individually and specifically described.

In some embodiments, provided herein is a compound of formula (A′), formula (A), or any variation of embodiment thereof, or a stereoisomer or tautomer thereof, or a pharmaceutically acceptable salt of any of the foregoing, wherein the compound is a compound of Table 1, or a stereoisomer or tautomer thereof, or a pharmaceutically acceptable salt of any of the foregoing.

TABLE 1

| Compound | | |
|---|---|---|
| No. | Structure | Chemical name |
| 1 | 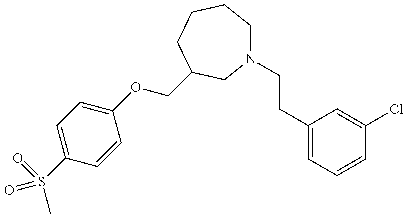 [SMILES: CS(=O)(=O)c1ccc(OCC2CCCCN(CCc3cccc(Cl)c3)C2)cc1] | 1-(3-chlorophenethyl)-3- ((4- (methylsulfonyl)phenoxy) methyl)azepane |
| 2 | 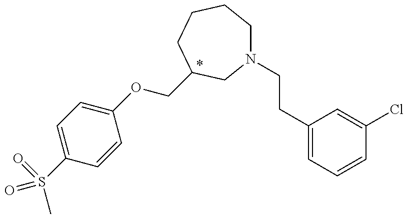 [SMILES: CS(=O)(=O)c1ccc(OCC2CCCCN(CCc3cccc(Cl)c3)C2)cc1] | (R) or (S)-1-(3- chlorophenethyl)-3-((4- (methylsulfonyl)phenoxy) methyl)azepane |
| 3 | 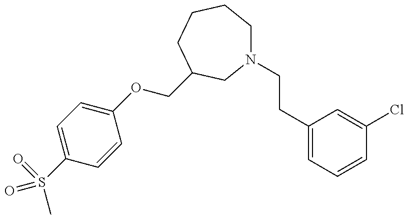 [SMILES: CS(=O)(=O)c1ccc(OCC2CCCCN(CCc3cccc(Cl)c3)C2)cc1] | (S) or (R)-1-(3- chlorophenethyl)-3-((4- (methylsulfonyl)phenoxy) methyl)azepane |
| 4 | 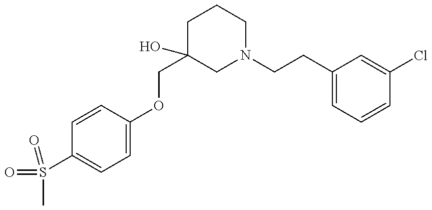 [SMILES: CS(=O)(=O)c1ccc(OCC2(O)CCCN(CCc3cccc(Cl)c3)C2)cc1] | 1-(3-chlorophenethyl)-3- ((4- (methylsulfonyl)phenoxy) methyl)piperidin-3-ol |
| 5 | 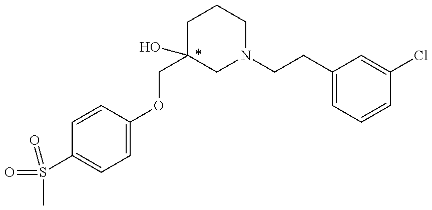 [SMILES: CS(=O)(=O)c1ccc(OCC2(O)CCCN(CCc3cccc(Cl)c3)C2)cc1] | (R) or (S)-1-(3- chlorophenethyl)-3-((4- (methylsulfonyl)phenoxy) methyl)piperidin-3-ol |
| 6 | 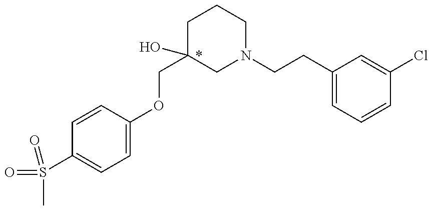 [SMILES: CS(=O)(=O)c1ccc(OCC2(O)CCCN(CCc3cccc(Cl)c3)C2)cc1] | (S) or (R) -1-(3- chlorophenethyl)-3-((4- (methylsulfonyl)phenoxy) methyl)piperidin-3-ol |
| 7 | 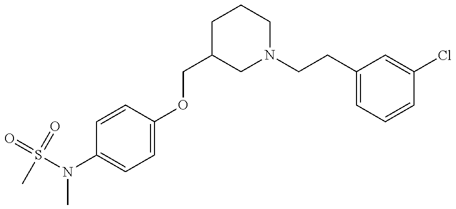 [SMILES: CN(c1ccc(OCC2CCCN(CCc3cccc(Cl)c3)C2)cc1)S(C)(=O)=O] | N-(4-((1-(3- chlorophenethyl)piperidin- 3-yl)methoxy)phenyl)-N- methylmethanesulfonamide |
| 8 | 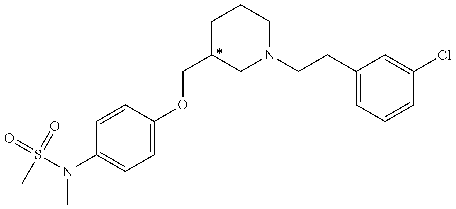 [SMILES: CN(c1ccc(OCC2CCCN(CCc3cccc(Cl)c3)C2)cc1)S(C)(=O)=O] | (S) or (R)-N-(4-((1-(3- chlorophenethyl)piperidin- 3-yl)methoxy)phenyl)-N- methylmethanesulfonamide |
| 9 | 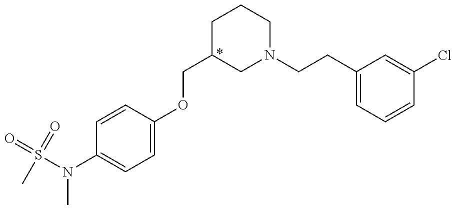 [SMILES: CN(c1ccc(OCC2CCCN(CCc3cccc(Cl)c3)C2)cc1)S(C)(=O)=O] | (R) or (S)-N-(4-((1-(3- chlorophenethyl)piperidin- 3-yl)methoxy)phenyl)-N- methylmethanesulfonamide |
| 10 | 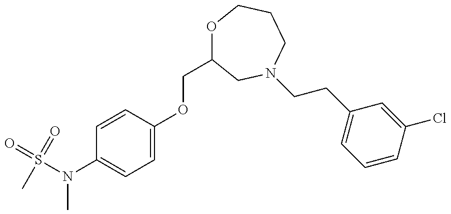 [SMILES: CN(c1ccc(OCC2CN(CCc3cccc(Cl)c3)CCCO2)cc1)S(C)(=O)=O] | N-(4-((4-(3- chlorophenethyl)-1,4- oxazepan-2- yl)methoxy)phenyl)-N- methylmethanesulfonamide |
| 11 | 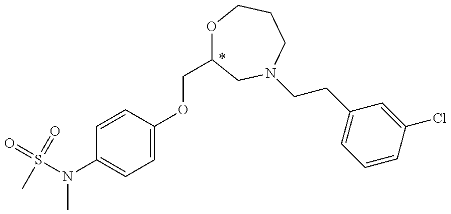 [SMILES: CN(c1ccc(OCC2CN(CCc3cccc(Cl)c3)CCCO2)cc1)S(C)(=O)=O] | (R) or (S)-N-(4-((4-(3- chlorophenethyl)-1,4- oxazepan-2- yl)methoxy)phenyl)-N- methylmethanesulfonamide |
| 12 | 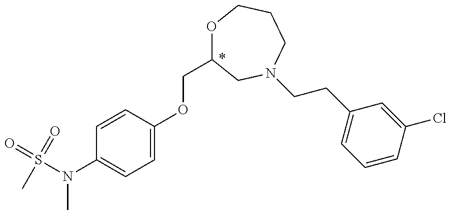 [SMILES: CN(c1ccc(OCC2CN(CCc3cccc(Cl)c3)CCCO2)cc1)S(C)(=O)=O] | (S) or (R)- N-(4-((4-(3- chlorophenethyl)-1,4- oxazepan-2- yl)methoxy)phenyl)-N- methylmethanesulfonamide |
| 13 | 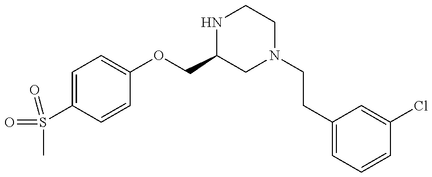 [SMILES: CS(=O)(=O)c1ccc(OC[C@@H]2CN(CCc3cccc(Cl)c3)CCN2)cc1] | (S)-1-(3-chlorophenethyl)- 3-((4- (methylsulfonyl)phenoxy) methyl)piperazine |
| 14 | 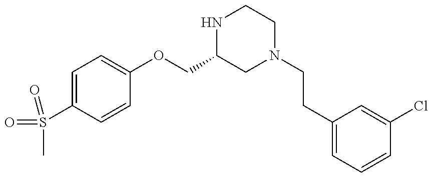 [SMILES: CS(=O)(=O)c1ccc(OC[C@H]2CN(CCc3cccc(Cl)c3)CCN2)cc1] | (R)-1-(3-chlorophenethyl)- 3-((4- (methylsulfonyl)phenoxy) methyl)piperazine |
| 15 | 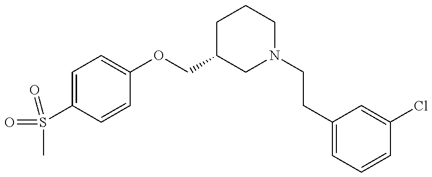 [SMILES: CS(=O)(=O)c1ccc(OC[C@@H]2CCCN(CCc3cccc(Cl)c3)C2)cc1] | (R)-1-(3-chlorophenethyl)- 3-((4- (methylsulfonyl)phenoxy) methyl)piperidine |
| 16 | 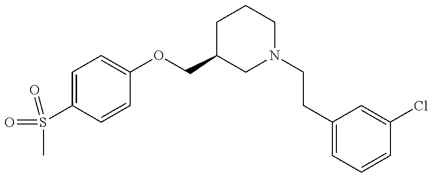 [SMILES: CS(=O)(=O)c1ccc(OC[C@H]2CCCN(CCc3cccc(Cl)c3)C2)cc1] | (S)-1-(3-chlorophenethyl)- 3-((4- (methylsulfonyl)phenoxy) methyl)piperidine |
| 17 | 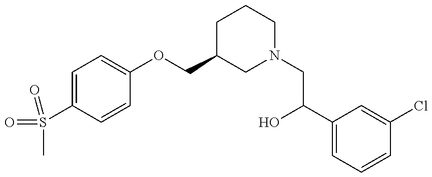 [SMILES: CS(=O)(=O)c1ccc(OC[C@H]2CCCN(CC(O)c3cccc(Cl)c3)C2)cc1] | 1-(3-chlorophenyl)-2-((S)-3- ((4- (methylsulfonyl)phenoxy) methyl)piperidin-1-yl)ethan- 1-ol |
| 18 | 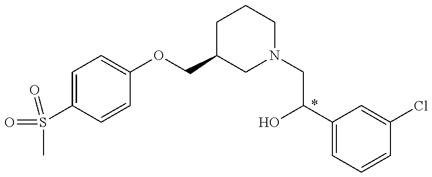 [SMILES: CS(=O)(=O)c1ccc(OC[C@H]2CCCN(CC(O)c3cccc(Cl)c3)C2)cc1] | (R) or (S)-1-(3- chlorophenyl)-2-((S)-3-((4- (methylsulfonyl)phenoxy) methyl)piperidin-1-yl)ethan- 1-ol |
| 19 | 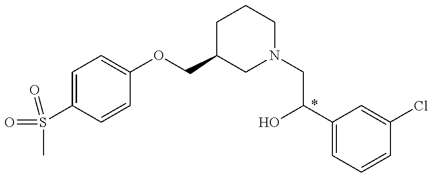 [SMILES: CS(=O)(=O)c1ccc(OC[C@H]2CCCN(CC(O)c3cccc(Cl)c3)C2)cc1] | (S) or (R)-1-(3- chlorophenyl)-2-((S)-3-((4- (methylsulfonyl)phenoxy) methyl)piperidin-1-yl)ethan- 1-ol |
| 20 | 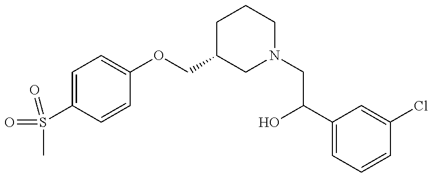 [SMILES: CS(=O)(=O)c1ccc(OC[C@@H]2CCCN(CC(O)c3cccc(Cl)c3)C2)cc1] | 1-(3-chlorophenyl)-2-((R)- 3-((4- (methylsulfonyl)phenoxy) methyl)piperidin-1-yl)ethanol |
| 21 | 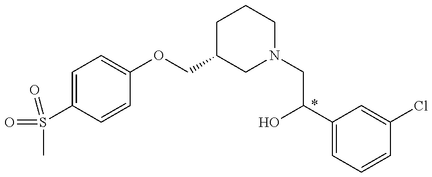 [SMILES: CS(=O)(=O)c1ccc(OC[C@@H]2CCCN(CC(O)c3cccc(Cl)c3)C2)cc1] | (R) or (S)-1-(3- chlorophenyl)-2-((R)-3-((4- (methylsulfonyl)phenoxy) methyl)piperidin-1-yl)ethanol |
| 22 | 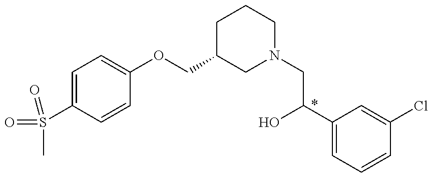 [SMILES: CS(=O)(=O)c1ccc(OC[C@@H]2CCCN(CC(O)c3cccc(Cl)c3)C2)cc1] | (S) or (R)-1-(3- chlorophenyl)-2-((R)-3-((4- (methylsulfonyl)phenoxy) methyl)piperidin-1-yl)ethanol |
| 23 | 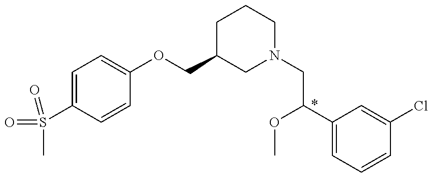 [SMILES: COC(CN1CCC[C@H](COc2ccc(S(C)(=O)=O)cc2)C1)c1cccc(Cl)c1] | (S)-1-((R) or (S)-2-(3- chlorophenyl)-2- methoxyethyl)-3-((4- (methylsulfonyl)phenoxy) methyl)piperidine |
| 24 | 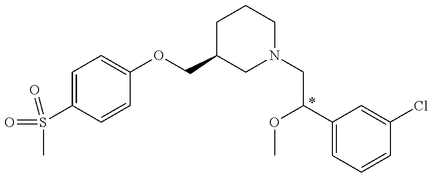 [SMILES: COC(CN1CCC[C@H](COc2ccc(S(C)(=O)=O)cc2)C1)c1cccc(Cl)c1] | (S)-1-((S) or (R)-2-(3- chlorophenyl)-2- methoxyethyl)-3-((4- (methylsulfonyl)phenoxy) methyl)piperidine |
| 25 | 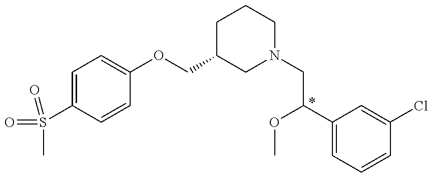 [SMILES: COC(CN1CCC[C@@H](COc2ccc(S(C)(=O)=O)cc2)C1)c1cccc(Cl)c1] | (R)-1-((R) or (S)-2-(3- chlorophenyl)-2- methoxyethyl)-3-((4- (methylsulfonyl)phenoxy) methyl)piperidine |
| 26 | 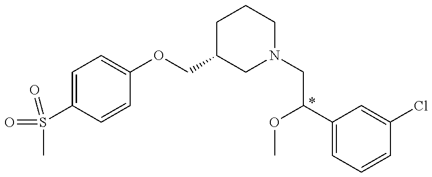 [SMILES: COC(CN1CCC[C@@H](COc2ccc(S(C)(=O)=O)cc2)C1)c1cccc(Cl)c1] | (R)-1-((S) or (R)-2-(3- chlorophenyl)-2- methoxyethyl)-3-((4- (methylsulfonyl)phenoxy) methyl)piperidine |
| 27 | 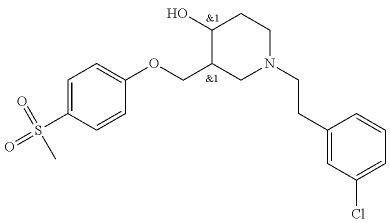 [SMILES: CS(=O)(=O)c1ccc(OCC2CN(CCc3cccc(Cl)c3)CCC2O)cc1] | rac-trans or cis-1-(3- chlorophenethyl)-3-((4- (methylsulfonyl)phenoxy) methyl)piperidin-4-ol |
| 28 | 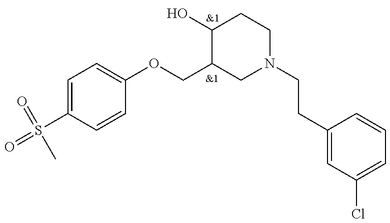 [SMILES: CS(=O)(=O)c1ccc(OCC2CN(CCc3cccc(Cl)c3)CCC2O)cc1] | rac-cis or trans-1-(3- chlorophenethyl)-3-((4- (methylsulfonyl)phenoxy) methyl)piperidin-4-ol |
| 29 | 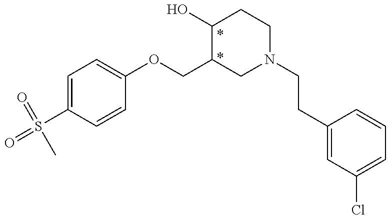 [SMILES: CS(=O)(=O)c1ccc(OCC2CN(CCc3cccc(Cl)c3)CCC2O)cc1] | (3S,4R) or (3R,4R) or (3S,4S) or (3R,4S)-1-(3- chlorophenethyl)-3-((4- (methylsulfonyl)phenoxy) methyl)piperidin-4-ol |
| 30 | 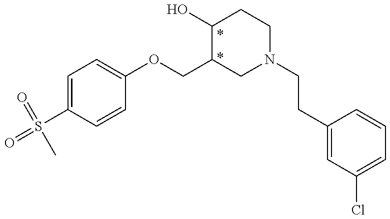 [SMILES: CS(=O)(=O)c1ccc(OCC2CN(CCc3cccc(Cl)c3)CCC2O)cc1] | (3R,4R) or (3S,4S) or (3R,4S) or (3S,4R) -1-(3- chlorophenethyl)-3-((4- (methylsulfonyl)phenoxy) methyl)piperidin-4-ol |
| 31 | 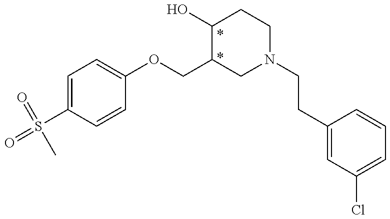 [SMILES: CS(=O)(=O)c1ccc(OCC2CN(CCc3cccc(Cl)c3)CCC2O)cc1] | (3R,4R) or (3S,4S) or (3R,4S) or (3S,4R) -1-(3- chlorophenethyl)-3-((4- (methylsulfonyl)phenoxy) methyl)piperidin-4-ol |
| 32 | 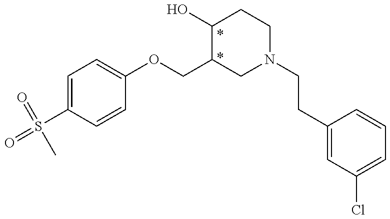 [SMILES: CS(=O)(=O)c1ccc(OCC2CN(CCc3cccc(Cl)c3)CCC2O)cc1] | (3S,4S) or (3R,4S) or (3S,4R) or (3R,4R)-1-(3- chlorophenethyl)-3-((4- (methylsulfonyl)phenoxy) methyl)piperidin-4-ol |
| 33 | 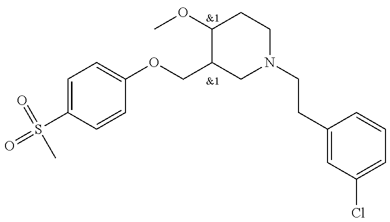 [SMILES: COC1CCN(CCc2cccc(Cl)c2)CC1COc1ccc(S(C)(=O)=O)cc1] | rac-trans or cis-1-(3- chlorophenethyl)-4- methoxy-3-((4- (methylsulfonyl) phenoxy) methyl)piperidine |
| 34 | 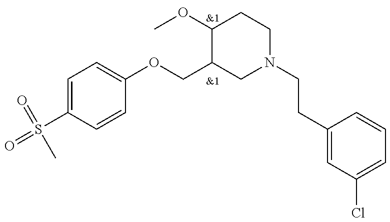 [SMILES: COC1CCN(CCc2cccc(Cl)c2)CC1COc1ccc(S(C)(=O)=O)cc1] | rac-cis or trans-1-(3- chlorophenethyl)-4- methoxy-3-((4- (methylsulfonyl)phenoxy) methyl)piperidine |
| 35 | 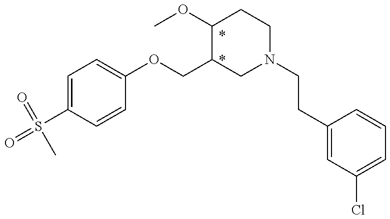 [SMILES: COC1CCN(CCc2cccc(Cl)c2)CC1COc1ccc(S(C)(=O)=O)cc1] | (3S,4R) or (3R,4R) or (3S,4S) or (3R,4S)-1-(3- chlorophenethyl)-4- methoxy-3-((4- (methylsulfonyl)phenoxy) methyl)piperidine |
| 36 | 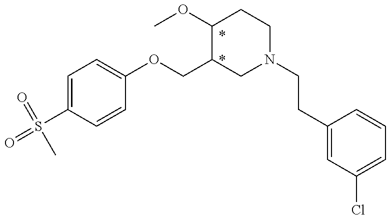 [SMILES: COC1CCN(CCc2cccc(Cl)c2)CC1COc1ccc(S(C)(=O)=O)cc1] | (3R,4R) or (3,4S) or (3R,4S) or (3S,4R)-1-(3- chlorophenethyl)-4- methoxy-3-((4- (methylsulfonyl)phenoxy) methyl)piperidine |
| 37 | 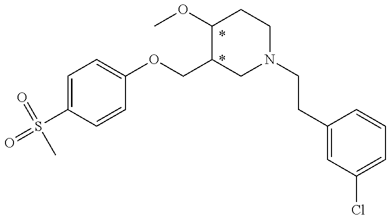 [SMILES: COC1CCN(CCc2cccc(Cl)c2)CC1COc1ccc(S(C)(=O)=O)cc1] | (3R,4R) or (3S,4S) or (3R,4S) or (3S,4R) -1-(3- chlorophenethyl)-4- methoxy-3-((4- (methylsulfonyl)phenoxy) methyl)piperidine |
| 38 | 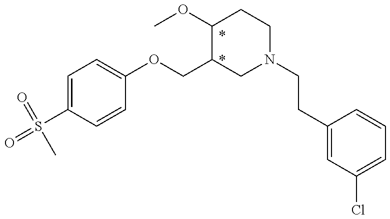 [SMILES: COC1CCN(CCc2cccc(Cl)c2)CC1COc1ccc(S(C)(=O)=O)cc1] | (3S,4S) or (3R,4S) or (3S,4R) or (3R,4R) -1-(3- chlorophenethyl)-4- methoxy-3-((4- (methylsulfonyl)phenoxy) methyl)piperidine |
| 39 | 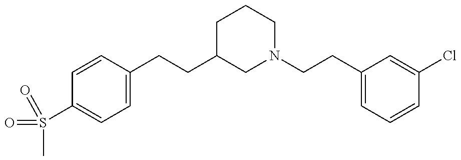 [SMILES: CS(=O)(=O)c1ccc(CCC2CCCN(CCc3cccc(Cl)c3)C2)cc1] | 1-(3-chlorophenethyl)-3-(4- (methylsulfonyl)phenethyl) piperidine |
| 40 | 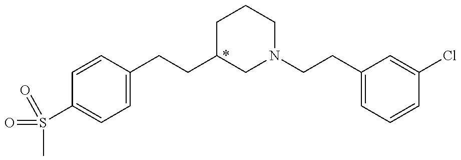 [SMILES: CS(=O)(=O)c1ccc(CCC2CCCN(CCc3cccc(Cl)c3)C2)cc1] | (S) or (R)-1-(3- chlorophenethyl)-3-(4- (methylsulfonyl)phenethyl) piperidine |
| 41 | 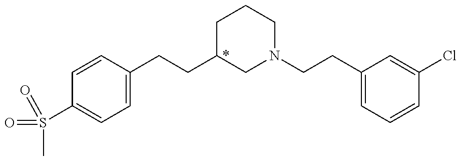 [SMILES: CS(=O)(=O)c1ccc(CCC2CCCN(CCc3cccc(Cl)c3)C2)cc1] | (R) or (S)-1-(3- chlorophenethyl)-3-(4- (methylsulfonyl)phenethyl) piperidine |
| 42 | 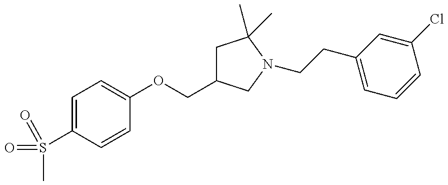 [SMILES: CC1(C)CC(COc2ccc(S(C)(=O)=O)cc2)CN1CCc1cccc(Cl)c1] | 1-(3-chlorophenethyl)-2,2- dimethyl-4-((4- (methylsulfonyl)phenoxy) methyl)pyrrolidine |
| 43 | 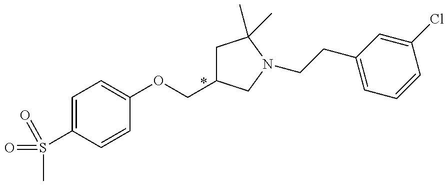 [SMILES: CC1(C)CC(COc2ccc(S(C)(=O)=O)cc2)CN1CCc1cccc(Cl)c1] | (S) or (R)-1-(3- chlorophenethyl)-2,2- dimethyl-4-((4- (methylsulfonyl)phenoxy) methyl)pyrrolidine |
| 44 | 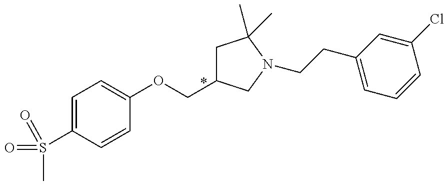 [SMILES: CC1(C)CC(COc2ccc(S(C)(=O)=O)cc2)CN1CCc1cccc(Cl)c1] | (R) or (S)-1-(3- chlorophenethyl)-2,2- dimethyl-4-((4- (methylsulfonyl)phenoxy) methyl)pyrrolidine |
| 45 | 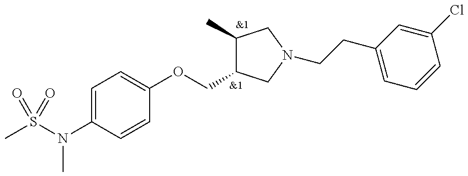 [SMILES: C[C@H]1CN(CCc2cccc(Cl)c2)C[C@@H]1COc1ccc(N(C)S(C)(=O)=O)cc1] | rac-trans-N-(4-((1-(3- chlorophenethyl)-4- methylpyrrolidin-3- yl)methoxy)phenyl)-N- methylmethanesulfonamide |
| 46 | 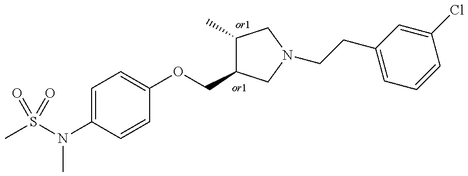 [SMILES: C[C@@H]1CN(CCc2cccc(Cl)c2)C[C@H]1COc1ccc(N(C)S(C)(=O)=O)cc1] | N-(4-(((3S,4S) or (3R,4R)- 1-(3-chlorophenethyl)-4- methylpyrrolidin-3- yl)methoxy)phenyl)-N- methylmethanesulfonamide |
| 47 | 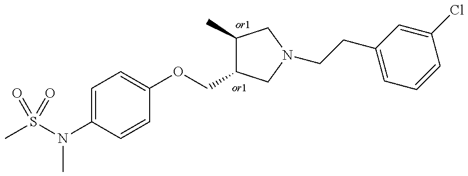 [SMILES: C[C@H]1CN(CCc2cccc(Cl)c2)C[C@@H]1COc1ccc(N(C)S(C)(=O)=O)cc1] | N-(4-(((3R,4R) or (3S,4S)- 1-(3-chlorophenethyl)-4- methylpyrrolidin-3- yl)methoxy)phenyl)-N- methylmethanesulfonamide |
| 48 | 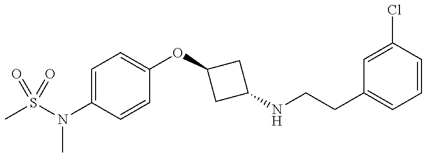 [SMILES: CN(c1ccc(O[C@H]2C[C@H](NCCc3cccc(Cl)c3)C2)cc1)S(C)(=O)=O] | trans-N-(4-(3-((3- chlorophenethyl)amino) cyclobutoxy)phenyl)-N- methylmethanesulfonamide |
| 49 | 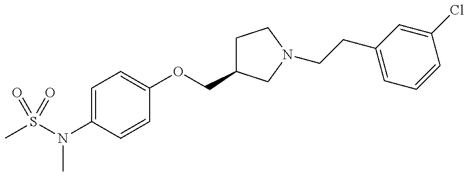 [SMILES: CN(c1ccc(OC[C@H]2CCN(CCc3cccc(Cl)c3)C2)cc1)S(C)(=O)=O] | (S)-N-(4-((1-(3- chlorophenethyl)pyrrolidin- 3-yl)methoxy)phenyl)-N- methylmethanesulfonamide |
| 50 | 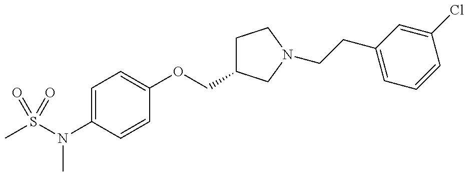 [SMILES: CN(c1ccc(OC[C@@H]2CCN(CCc3cccc(Cl)c3)C2)cc1)S(C)(=O)=O] | (R)-N-(4-((1-(3- chlorophenethyl)pyrrolidin- 3-yl)methoxy)phenyl)-N- methylmethanesulfonamide |
| 51 | 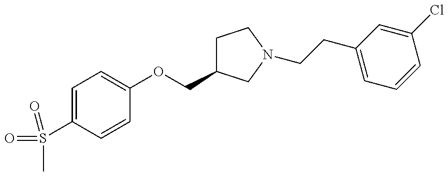 [SMILES: CS(=O)(=O)c1ccc(OC[C@H]2CCN(CCc3cccc(Cl)c3)C2)cc1] | (S)-1-(3-chlorophenethyl)- 3-((4- (methylsulfonyl)phenoxy) methyl)pyrrolidine |
| 52 | 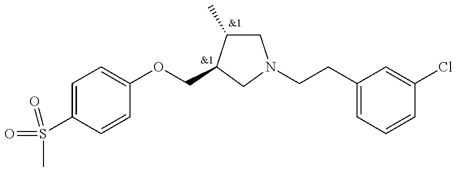 [SMILES: C[C@@H]1CN(CCc2cccc(Cl)c2)C[C@H]1COc1ccc(S(C)(=O)=O)cc1] | rac-trans-1-(3- chlorophenethyl)-3-methyl- 4-((4- (methylsulfonyl)phenoxy) methyl)pyrrolidine |
| 53 | 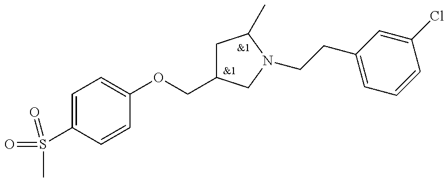 [SMILES: CC1CC(COc2ccc(S(C)(=O)=O)cc2)CN1CCc1cccc(Cl)c1] | rac-trans or cis-1-(3- chlorophenethyl)-2-methyl- 4-((4- (methylsulfonyl)phenoxy) methyl)pyrrolidine |
| 54 | 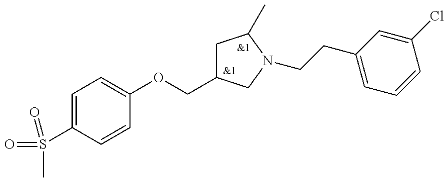 [SMILES: CC1CC(COc2ccc(S(C)(=O)=O)cc2)CN1CCc1cccc(Cl)c1] | rac-cis or trans-1-(3- chlorophenethyl)-2-methyl- 4-((4- (methylsulfonyl)phenoxy) methyl)pyrrolidine |
| 55 | 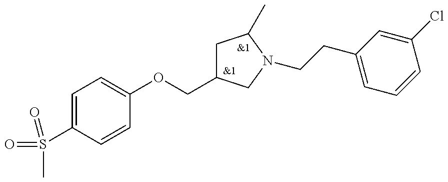 [SMILES: CC1CC(COc2ccc(S(C)(=O)=O)cc2)CN1CCc1cccc(Cl)c1] | (2R,4S) or (2R,4R) or (2S,4R) or (2S,4S)-1-(3- chlorophenethyl)-2-methyl- 4-((4- (methylsulfonyl)phenoxy) methyl)pyrrolidine |
| 56 | 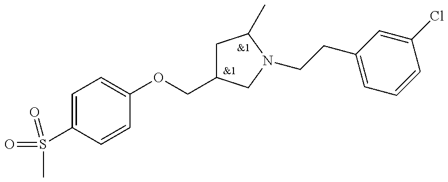 [SMILES: CC1CC(COc2ccc(S(C)(=O)=O)cc2)CN1CCc1cccc(Cl)c1] | (2R,4R) or (2S,4R) or (2S,4S) or (2R,4S)-1-(3- chlorophenethyl)-2-methyl- 4-((4- (methylsulfonyl)phenoxy) methyl)pyrrolidine |
| 57 | 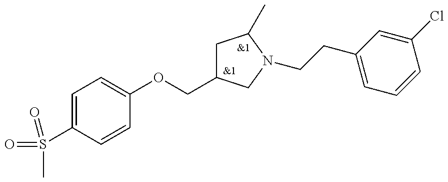 [SMILES: CC1CC(COc2ccc(S(C)(=O)=O)cc2)CN1CCc1cccc(Cl)c1] | (2S,4R) or (2S,4S) or (2R,4S) or (2R,4R)-1-(3- chlorophenethyl)-2-methyl- 4-((4- (methylsulfonyl)phenoxy) methyl)pyrrolidine |
| 58 | 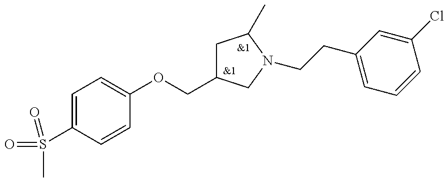 [SMILES: CC1CC(COc2ccc(S(C)(=O)=O)cc2)CN1CCc1cccc(Cl)c1] | (2S,4S) or (2R,4S) or (2R,4R) or (2S,4R)-1-(3- chlorophenethyl)-2-methyl- 4-((4- (methylsulfonyl)phenoxy) methyl)pyrrolidine |
| 59 | 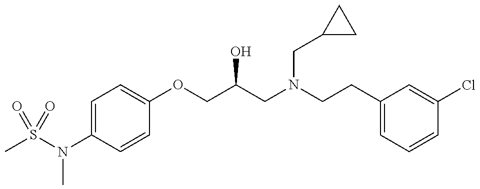 [SMILES: CN(c1ccc(OC[C@@H](O)CN(CCc2cccc(Cl)c2)CC2CC2)cc1)S(C)(=O)=O] | (S)-N-(4-(3-((3- chlorophenethyl) (cyclopropylmethyl)amino)-2- hydroxypropoxy)phenyl)-N- methylmethanesulfonamide |
| 60 | 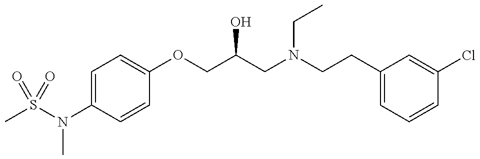 [SMILES: CCN(CCc1cccc(Cl)c1)C[C@H](O)COc1ccc(N(C)S(C)(=O)=O)cc1] | (S)-N-(4-(3-((3- chlorophenethyl)(ethyl) amino)-2- hydroxypropoxy)phenyl)-N- methylmethanesulfonamide |
| 61 | 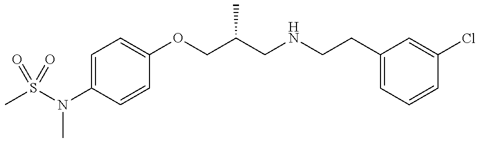 [SMILES: C[C@H](CNCCc1cccc(Cl)c1)COc1ccc(N(C)S(C)(=O)=O)cc1] | (R)-N-(4-(3-((3- chlorophenethyl)amino)-2- methylpropoxy)phenyl)-N- methylmethanesulfonamide |
| 62 | 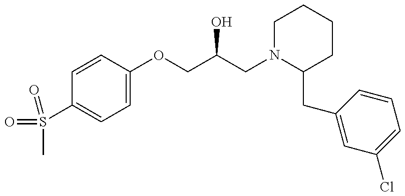 [SMILES: CS(=O)(=O)c1ccc(OC[C@@H](O)CN2CCCCC2Cc2cccc(Cl)c2)cc1] | (2S)-1-(2-(3- chlorobenzyl)piperidin-1- yl)-3-(4- (methylsulfonyl)phenoxy) propan-2-ol |
| 63 | 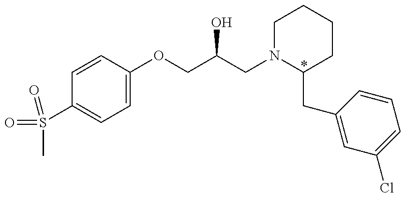 [SMILES: CS(=O)(=O)c1ccc(OC[C@@H](O)CN2CCCCC2Cc2cccc(Cl)c2)cc1] | (S)-1-((S) or (R)-2-(3- chlorobenzyl)piperidin-1- yl)-3-(4- (methylsulfonyl)phenoxy) propan-2-ol |
| 64 | 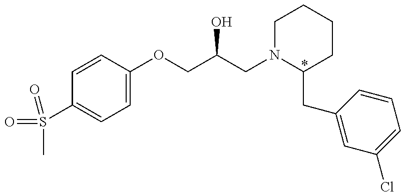 [SMILES: CS(=O)(=O)c1ccc(OC[C@@H](O)CN2CCCCC2Cc2cccc(Cl)c2)cc1] | (S)-1-((R) or (S)-2-(3- chlorobenzyl)piperidin-1- yl)-3-(4- (methylsulfonyl)phenoxy) propan-2-ol |
| 65 | 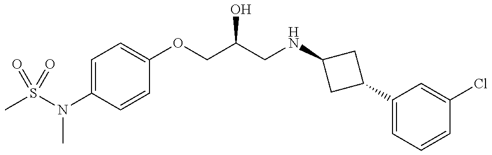 [SMILES: CN(c1ccc(OC[C@@H](O)CN[C@H]2C[C@H](c3cccc(Cl)c3)C2)cc1)S(C)(=O)=O] | N-(4-((S)-3-((trans-3-(3- chlorophenyl)cyclobutyl) amino)-2- hydroxypropoxy)phenyl)-N- methylmethanesulfonamide |
| 66 | 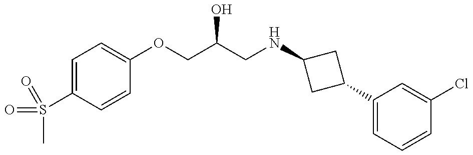 [SMILES: CS(=O)(=O)c1ccc(OC[C@@H](O)CN[C@H]2C[C@H](c3cccc(Cl)c3)C2)cc1] | (S)-1-((trans-3-(3- chlorophenyl)cyclobutyl) amino)-3-(4- (methylsulfonyl)phenoxy) propan-2-ol |
| 67 | 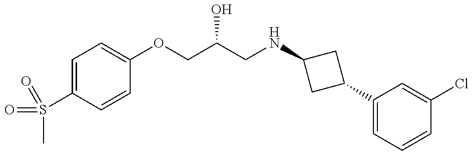 [SMILES: CS(=O)(=O)c1ccc(OC[C@H](O)CN[C@H]2C[C@H](c3cccc(Cl)c3)C2)cc1] | (R)-1-((trans-3-(3- chlorophenyl)cyclobutyl) amino)-3-(4- (methylsulfonyl)phenoxy) propan-2-ol |
| 68 | 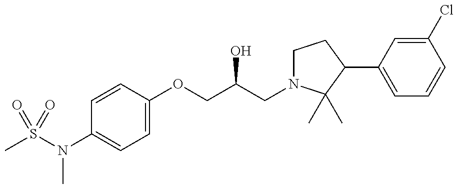 [SMILES: CN(c1ccc(OC[C@@H](O)CN2CCC(c3cccc(Cl)c3)C2(C)C)cc1)S(C)(=O)=O] | N-(4-((2S)-3-(3-(3- chlorophenyl)-2,2- dimethylpyrrolidin-1-yl)-2- hydroxypropoxy)phenyl)-N- methylmethanesulfonamide |
| 69 | 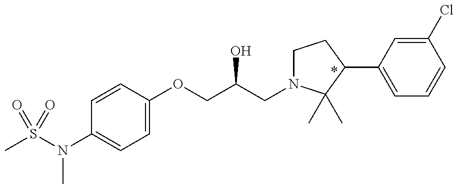 [SMILES: CN(c1ccc(OC[C@@H](O)CN2CCC(c3cccc(Cl)c3)C2(C)C)cc1)S(C)(=O)=O] | N-(4-((S)-3-((S) or (R)-3-(3- chlorophenyl)-2,2- dimethylpyrrolidin-1-yl)-2- hydroxypropoxy)phenyl)-N- methylmethanesulfonamide |
| 70 | 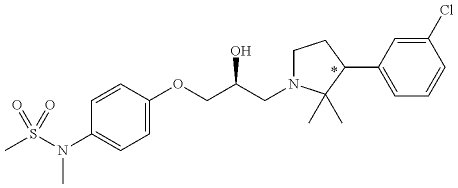 [SMILES: CN(c1ccc(OC[C@@H](O)CN2CCC(c3cccc(Cl)c3)C2(C)C)cc1)S(C)(=O)=O] | N-(4-((S)-3-((R) or (S)-3-(3- chlorophenyl)-2,2- dimethylpyrrolidin-1-yl)-2- hydroxypropoxy)phenyl)-N- methylmethanesulfonamide |
| 71 | 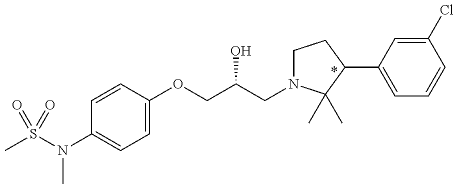 [SMILES: CN(c1ccc(OC[C@H](O)CN2CCC(c3cccc(Cl)c3)C2(C)C)cc1)S(C)(=O)=O] | N-(4-((2R)-3-(3-(3- chlorophenyl)-2,2- dimethylpyrrolidin-1-yl)-2- hydroxypropoxy)phenyl)-N- methylmethanesulfonamide |
| 72 | 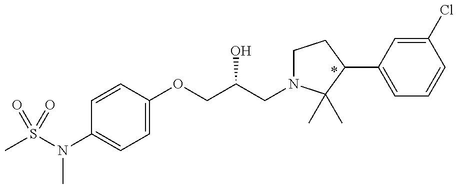 [SMILES: CN(c1ccc(OC[C@H](O)CN2CCC(c3cccc(Cl)c3)C2(C)C)cc1)S(C)(=O)=O] | N-(4-((R)-3-((S) or (R)-3-(3- chlorophenyl)-2,2- dimethylpyrrolidin-1-yl)-2- hydroxypropoxy)phenyl)-N- methylmethanesulfonamide |
| 73 | 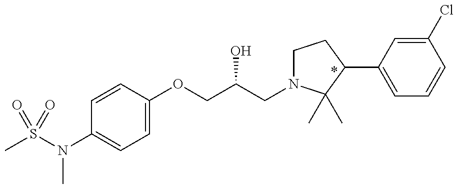 [SMILES: CN(c1ccc(OC[C@H](O)CN2CCC(c3cccc(Cl)c3)C2(C)C)cc1)S(C)(=O)=O] | N-(4-((R)-3-((R) or (S)-3-(3- chlorophenyl)-2,2- dimethylpyrrolidin-1-yl)-2- hydroxypropoxy)phenyl)-N- methylmethanesulfonamide |
| 74 | 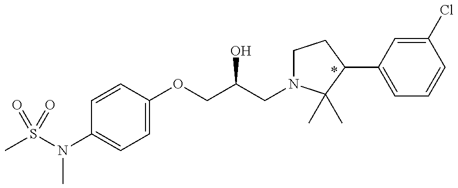 [SMILES: CN(c1ccc(OC[C@@H](O)CN2CCC(c3cccc(Cl)c3)C2(C)C)cc1)S(C)(=O)=O] | N-(4-((2S)-3-(3-(3- chlorophenyl)pyrrolidin-1- yl)-2- hydroxypropoxy)phenyl)-N- methylmethanesulfonamide |
| 75 | 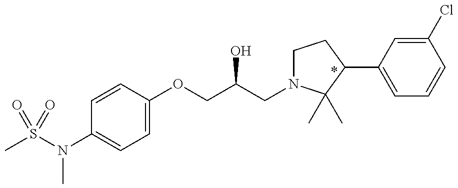 [SMILES: CN(c1ccc(OC[C@@H](O)CN2CCC(c3cccc(Cl)c3)C2(C)C)cc1)S(C)(=O)=O] | N-(4-((S)-3-((S) or (R)-3-(3- chlorophenyl)pyrrolidin-1- yl)-2- hydroxypropoxy)phenyl)-N- methylmethanesulfonamide |
| 76 | 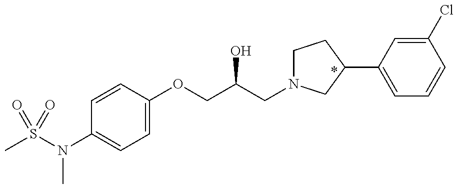 [SMILES: CN(c1ccc(OC[C@@H](O)CN2CCC(c3cccc(Cl)c3)C2)cc1)S(C)(=O)=O] | N-(4-((S)-3-((R) or (S)-3-(3- chlorophenyl)pyrrolidin-1- yl)-2- hydroxypropoxy)phenyl)-N- methylmethanesulfonamide |
| 77 | 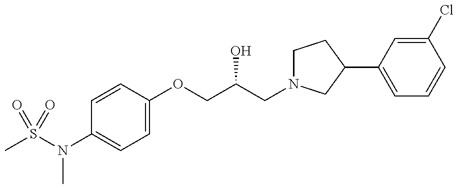 [SMILES: CN(c1ccc(OC[C@H](O)CN2CCC(c3cccc(Cl)c3)C2)cc1)S(C)(=O)=O] | N-(4-((2R)-3-(3-(3- chlorophenyl)pyrrolidin-1- yl)-2- hydroxypropoxy)phenyl)-N- methylmethanesulfonamide |
| 78 | 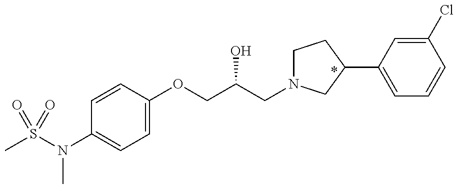 [SMILES: CN(c1ccc(OC[C@H](O)CN2CCC(c3cccc(Cl)c3)C2)cc1)S(C)(=O)=O] | N-(4-((R)-3-((S) or (R)-3-(3- chlorophenyl)pyrrolidin-1- yl)-2- hydroxypropoxy)phenyl)-N- methylmethanesulfonamide |
| 79 | 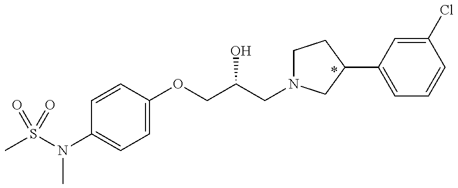 [SMILES: CN(c1ccc(OC[C@H](O)CN2CCC(c3cccc(Cl)c3)C2)cc1)S(C)(=O)=O] | N-(4-((R)-3-((R) or (S)-3-(3- chlorophenyl)pyrrolidin-1- yl)-2- hydroxypropoxy)phenyl)-N- methylmethanesulfonamide |
| 80 | 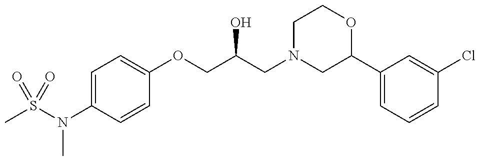 [SMILES: CN(c1ccc(OC[C@@H](O)CN2CCOC(c3cccc(Cl)c3)C2)cc1)S(C)(=O)=O] | N-(4-((2S)-3-(2-(3- chlorophenyl)morpholino)- 2-hydroxypropoxy)phenyl)- N- methylmethanesulfonamide |
| 81 | 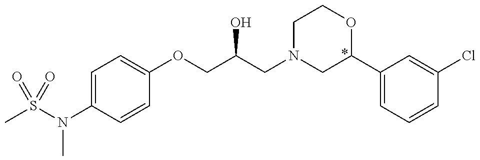 [SMILES: CN(c1ccc(OC[C@@H](O)CN2CCOC(c3cccc(Cl)c3)C2)cc1)S(C)(=O)=O] | N-(4-((S)-3-((S) or (R)-2-(3- chlorophenyl)morpholino)- 2-hydroxypropoxy)phenyl)- N- methylmethanesulfonamide |
| 82 | 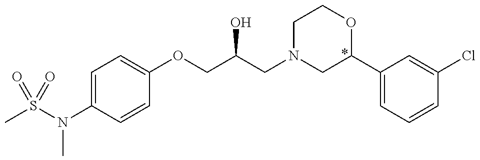 [SMILES: CN(c1ccc(OC[C@@H](O)CN2CCOC(c3cccc(Cl)c3)C2)cc1)S(C)(=O)=O] | N-(4-((S)-3-((R) or (S)-2-(3- chlorophenyl)morpholino)- 2-hydroxypropoxy)phenyl)- N- methylmethanesulfonamide |
| 83 | 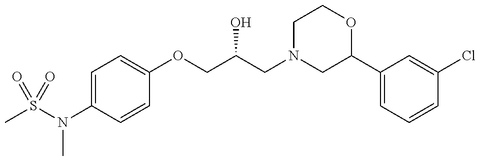 [SMILES: CN(c1ccc(OC[C@H](O)CN2CCOC(c3cccc(Cl)c3)C2)cc1)S(C)(=O)=O] | N-(4-((2R)-3-(2-(3- chlorophenyl)morpholino)- 2-hydroxypropoxy)phenyl)- N- methylmethanesulfonamide |
| 84 | 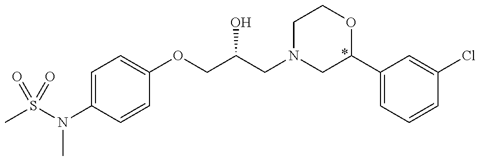 [SMILES: CN(c1ccc(OC[C@H](O)CN2CCOC(c3cccc(Cl)c3)C2)cc1)S(C)(=O)=O] | N-(4-((R)-3-((R) or (S)-2-(3- chlorophenyl)morpholino)- 2-hydroxypropoxy)phenyl)- N- methylmethanesulfonamide |
| 85 | 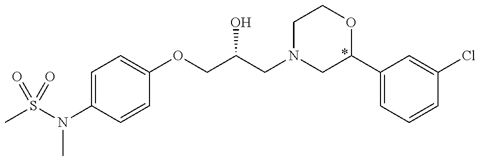 [SMILES: CN(c1ccc(OC[C@H](O)CN2CCOC(c3cccc(Cl)c3)C2)cc1)S(C)(=O)=O] | N-(4-((R)-3-((S) or (R)-2-(3- chlorophenyl)morpholino)- 2-hydroxypropoxy)phenyl)- N- methylmethanesulfonamide |
| 86 | 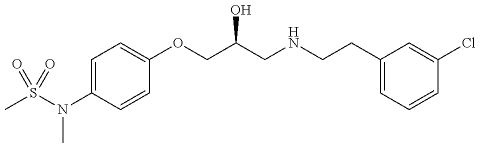 [SMILES: CN(c1ccc(OC[C@@H](O)CNCCc2cccc(Cl)c2)cc1)S(C)(=O)=O] | (S)-N-(4-(3-((3- chlorophenethyl)amino)-2- hydroxypropoxy)phenyl)-N- methylmethanesulfonamide |
| 87 | 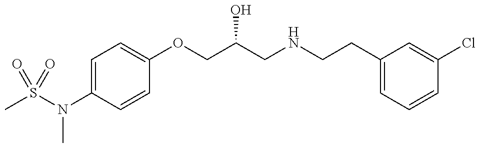 [SMILES: CN(c1ccc(OC[C@H](O)CNCCc2cccc(Cl)c2)cc1)S(C)(=O)=O] | (R)-N-(4-(3-((3- chlorophenethyl)amino)-2- hydroxypropoxy)phenyl)-N- methylmethanesulfonamide |
| 88 | 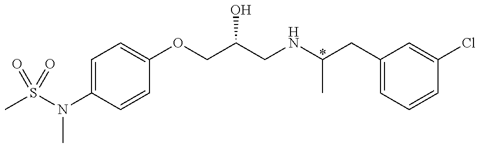 [SMILES: CC(Cc1cccc(Cl)c1)NC[C@@H](O)COc1ccc(N(C)S(C)(=O)=O)cc1] | N-(4-((R)-3-(((S) or (R)-1- (3-chlorophenyl)propan-2- yl)amino)-2- hydroxypropoxy)phenyl)-N- methylmethanesulfonamide |
| 89 | 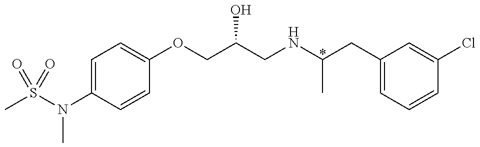 [SMILES: CC(Cc1cccc(Cl)c1)NC[C@@H](O)COc1ccc(N(C)S(C)(=O)=O)cc1] | N-(4-((R)-3-(((R) or (S)-1- (3-chlorophenyl)propan-2- yl)amino)-2- hydroxypropoxy)phenyl)-N- methylmethanesulfonamide |
| 90 | 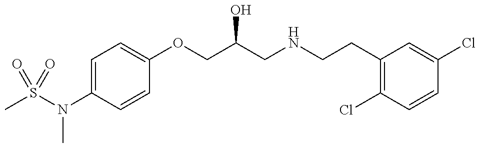 [SMILES: CN(c1ccc(OC[C@@H](O)CNCCc2cc(Cl)ccc2Cl)cc1)S(C)(=O)=O] | (S)-N-(4-(3-((2,5- dichlorophenethyl)amino)- 2-hydroxypropoxy)phenyl)- N- methylmethanesulfonamide |
| 91 | 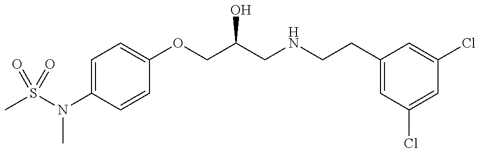 [SMILES: CN(c1ccc(OC[C@@H](O)CNCCc2cc(Cl)cc(Cl)c2)cc1)S(C)(=O)=O] | (S)-N-(4-(3-((3,5- dichlorophenethyl)amino)- 2-hydroxypropoxy)phenyl)- N- methylmethanesulfonamide |
| 92 | 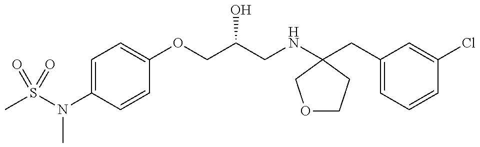 [SMILES: CN(c1ccc(OC[C@H](O)CNC2(Cc3cccc(Cl)c3)CCOC2)cc1)S(C)(=O)=O] | N-(4-((2R)-3-((3-(3- chlorobenzyl) tetrahydrofuran- 3-yl)amino)-2- hydroxypropoxy)phenyl)-N- methylmethanesulfonamide |
| 93 | 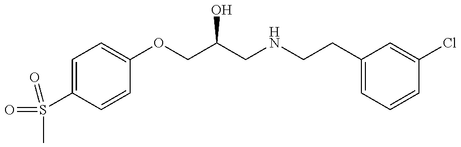 [SMILES: CS(=O)(=O)c1ccc(OC[C@@H](O)CNCCc2cccc(Cl)c2)cc1] | (S)-1-((3- chlorophenethyl)amino)-3- (4- (methylsulfonyl)phenoxy) propan-2-ol |
| 94 | 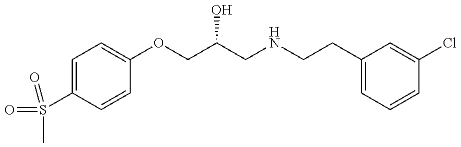 [SMILES: CS(=O)(=O)c1ccc(OC[C@H](O)CNCCc2cccc(Cl)c2)cc1] | (R)-1-((3- chlorophenethyl)amino)-3- (4- (methylsulfonyl)phenoxy) propan-2-ol |
| 95 | 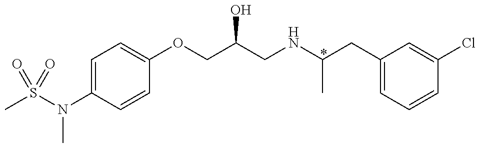 [SMILES: CC(Cc1cccc(Cl)c1)NC[C@H](O)COc1ccc(N(C)S(C)(=O)=O)cc1] | N-(4-((S)-3-(((S) or (R)-1- (3-chlorophenyl)propan-2- yl)amino)-2- hydroxypropoxy)phenyl)-N- methylmethanesulfonamide |
| 96 | 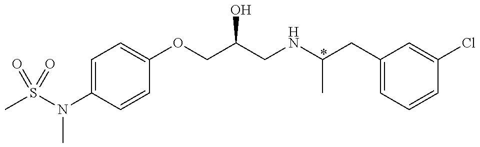 [SMILES: CC(Cc1cccc(Cl)c1)NC[C@H](O)COc1ccc(N(C)S(C)(=O)=O)cc1] | N-(4-((S)-3-(((R) or (S)-1- (3-chlorophenyl)propan-2- yl)amino)-2- hydroxypropoxy)phenyl)-N- methylmethanesulfonamide |
| 97 | 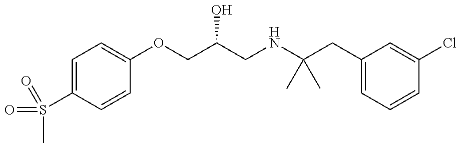 [SMILES: CC(C)(Cc1cccc(Cl)c1)NC[C@@H](O)COc1ccc(S(C)(=O)=O)cc1] | (R)-1-((1-(3-chlorophenyl)- 2-methylpropan-2- yl)amino)-3-(4- (methylsulfonyl)phenoxy) propan-2-ol |
| 98 | 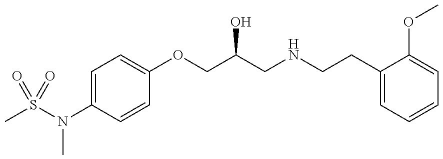 | (S)-N-(4-(2-hydroxy-3-((2- methoxyphenethyl)amino) propoxy)phenyl)-N- methylmethanesulfonamide |
| 99 | 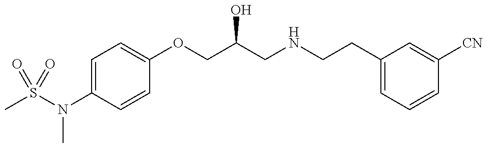 [SMILES: CN(c1ccc(OC[C@@H](O)CNCCc2cccc(C#N)c2)cc1)S(C)(=O)=O] | (S)-N-(4-(3-((3- cyanophenethyl)amino)-2- hydroxypropoxy)phenyl)-N- methylmethanesulfonamide |
| 100 | 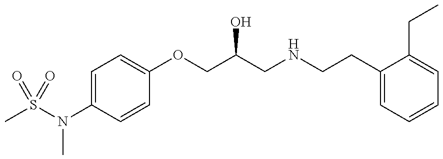 [SMILES: CCc1ccccc1CCNC[C@H](O)COc1ccc(N(C)S(C)(=O)=O)cc1] | (S)-N-(4-(3-((2- ethylphenethyl)amino)-2- hydroxypropoxy)phenyl)-N- methylmethanesulfonamide |
| 101 | 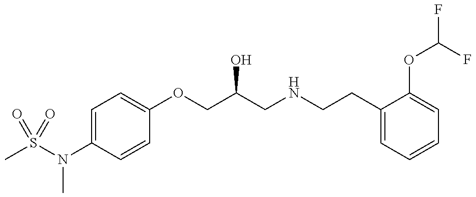 [SMILES: CN(c1ccc(OC[C@@H](O)CNCCc2ccccc2OC(F)F)cc1)S(C)(=O)=O] | (S)-N-(4-(3-((2- (difluoromethoxy)phenethyl) amino)-2- hydroxypropoxy)phenyl)-N- methylmethanesulfonamide |
| 102 | 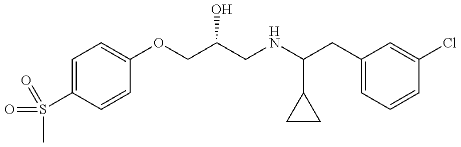 [SMILES: CS(=O)(=O)c1ccc(OC[C@H](O)CNC(Cc2cccc(Cl)c2)C2CC2)cc1] | (2R)-1-((2-(3- chlorophenyl)-1- cyclopropylethyl)amino)-3- (4- (methylsulfonyl)phenoxy) propan-2-ol |
| 103 | 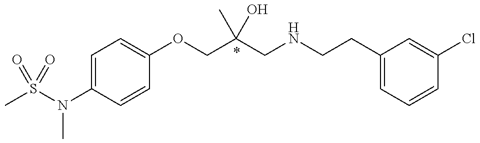 [SMILES: CN(c1ccc(OCC(C)(O)CNCCc2cccc(Cl)c2)cc1)S(C)(=O)=O] | (R) or (S)-N-(4-(3-((3- chlorophenethyl)amino)-2- hydroxy-2- methylpropoxy)phenyl)-N- methylmethanesulfonamide |
| 104 | 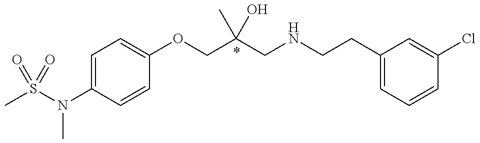 [SMILES: CN(c1ccc(OCC(C)(O)CNCCc2cccc(Cl)c2)cc1)S(C)(=O)=O] | (S) or (R)-N-(4-(3-((3- chlorophenethyl)amino)-2- hydroxy-2- methylpropoxy)phenyl)-N- methylmethanesulfonamide |
| 105 | 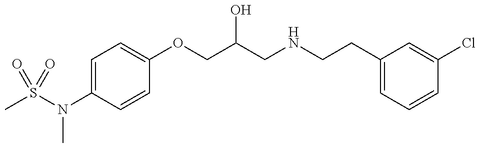 [SMILES: CN(c1ccc(OCC(O)CNCCc2cccc(Cl)c2)cc1)S(C)(=O)=O] | N-(4-((3-((3- chlorophenethyl)amino)-2- hydroxypropyl)amino) phenyl)-N- methylmethanesulfonamide |
| 106 | 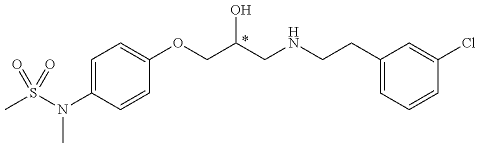 [SMILES: CN(c1ccc(OCC(O)CNCCc2cccc(Cl)c2)cc1)S(C)(=O)=O] | (R) or (S)-N-(4-((3-((3- chlorophenethyl)amino)-2- hydroxypropyl)amino) phenyl)-N- methylmethanesulfonamide |
| 107 | 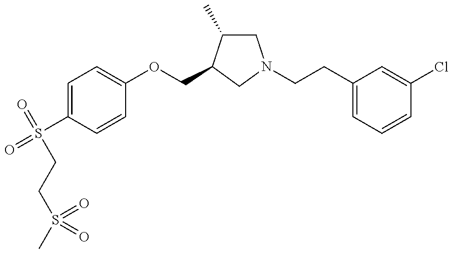 [SMILES: C[C@@H]1CN(CCc2cccc(Cl)c2)C[C@H]1COc1ccc(S(=O)(=O)CCS(C)(=O)=O)cc1] | (3S,4S)-1-[2-(3- chlorophenyl)ethyl]-3-{[4- (2- methanesulfonylethanesulfonyl) phenoxy]methyl}-4- methylpyrrolidine |
| 108 | 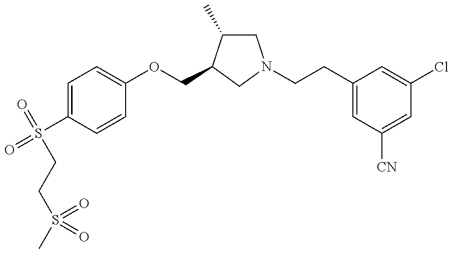 [SMILES: C[C@@H]1CN(CCc2cc(Cl)cc(C#N)c2)C[C@H]1COc1ccc(S(=O)(=O)CCS(C)(=O)=O)cc1] | 3-chloro-5-{2-[(3S,4S)-3- {[4-(2- methanesulfonylethanesulfonyl) phenoxy]methyl}-4- methylpyrrolidin-1- yl]ethyl}benzonitrile |
| 109 | 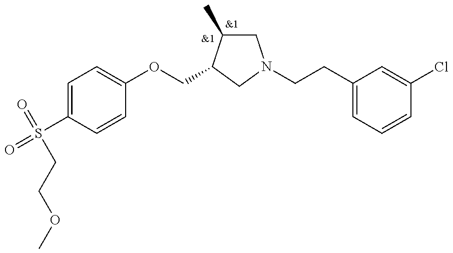 [SMILES: COCCS(=O)(=O)c1ccc(OC[C@H]2CN(CCc3cccc(Cl)c3)C[C@@H]2C)cc1] | rac-trans-1-[2-(3- chlorophenyl)ethyl]-3- {[4-(2- methoxyethanesulfonyl) phenoxy]methyl}-4- methylpyrrolidine |
| 110 | 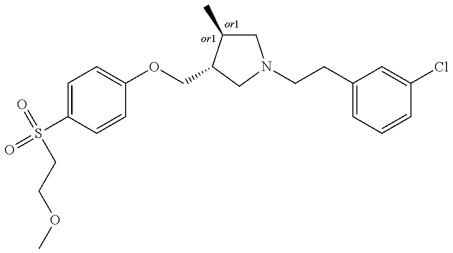 [SMILES: COCCS(=O)(=O)c1ccc(OC[C@H]2CN(CCc3cccc(Cl)c3)C[C@@H]2C)cc1] | (3R,4R or 3S,4S)-1-[2-(3- chlorophenyl)ethyl]-3-{[4- (2- methoxyethanesulfonyl) phenoxy]methyl}-4- methylpyrrolidine |
| 111 | 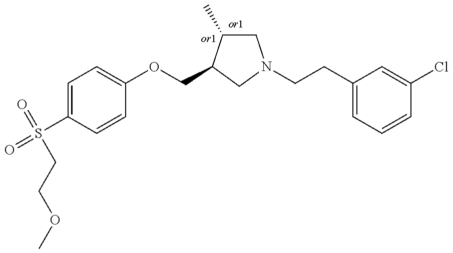 [SMILES: COCCS(=O)(=O)c1ccc(OC[C@@H]2CN(CCc3cccc(Cl)c3)C[C@H]2C)cc1] | (3S,4S or 3R,4R)-1-[2-(3- chlorophenyl)ethyl]-3-{[4-(2- methoxyethanesulfonyl) phenoxy]methyl}-4- methylpyrrolidine |
| 112 | 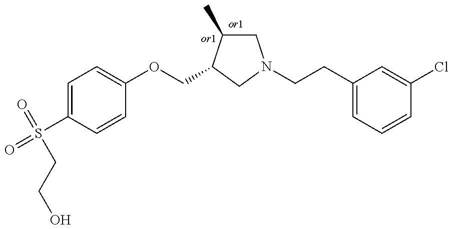 [SMILES: C[C@H]1CN(CCc2cccc(Cl)c2)C[C@@H]1COc1ccc(S(=O)(=O)CCO)cc1] | 2-(4-{[(3R,4R or 3S,4S)-1- [2-(3-chlorophenyl)ethyl]-4- methylpyrrolidin-3- yl]methoxy}benzenesulfonyl) ethan-1-ol |
| 113 | 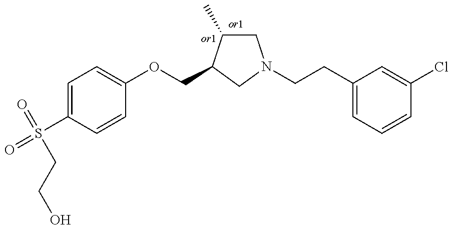 [SMILES: C[C@@H]1CN(CCc2cccc(Cl)c2)C[C@H]1COc1ccc(S(=O)(=O)CCO)cc1] | 2-(4-{[(3S,4S or 3R,4R)-1- [2-(3-chlorophenyl)ethyl]-4- methylpyrrolidin-3- yl]methoxy}benzenesulfonyl) ethan-1-ol |
| 114 | 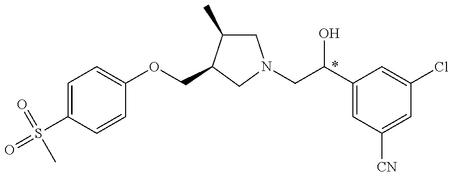 [SMILES: C[C@H]1CN(CC(O)c2cc(Cl)cc(C#N)c2)C[C@H]1COc1ccc(S(C)(=O)=O)cc1] | 3-chloro-5-[(1S or 1R)-1- hydroxy-2-[(2R,4S)-4-[(4- methanesulfonylphenoxy) methyl]-2-methylpyrrolidin-1- yl]ethyl]benzonitrile |
| 115 | 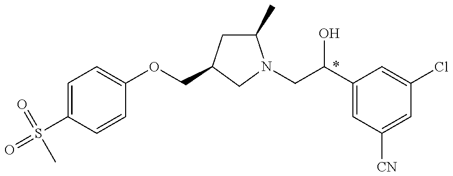 [SMILES: C[C@@H]1C[C@H](COc2ccc(S(C)(=O)=O)cc2)CN1CC(O)c1cc(Cl)cc(C#N)c1] | 3-chloro-5-[(1R or 1S)-1- hydroxy-2-[(2R,4S)-4-[(4- methanesulfonylphenoxy) methyl]-2-methylpyrrolidin-1- yl]ethyl]benzonitrile |
| 116 | 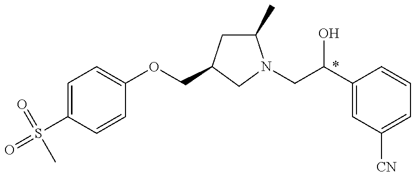 [SMILES: C[C@@H]1C[C@H](COc2ccc(S(C)(=O)=O)cc2)CN1CC(O)c1cccc(C#N)c1] | 3-[(1R or 1S)-1-hydroxy-2- [(2R,4S)-4-[(4- methanesulfonylphenoxy) methyl]-2-methylpyrrolidin-1- yl]ethyl]benzonitrile |
| 117 | 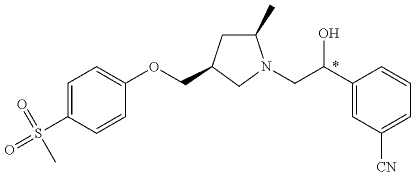 [SMILES: C[C@@H]1C[C@H](COc2ccc(S(C)(=O)=O)cc2)CN1CC(O)c1cccc(C#N)c1] | 3-[(1S or 1R)-1-hydroxy-2- [(2R,4S)-4-[(4- methanesulfonylphenoxy) methyl]-2-methylpyrrolidin-1- yl]ethyl]benzonitrile |
| 118 | 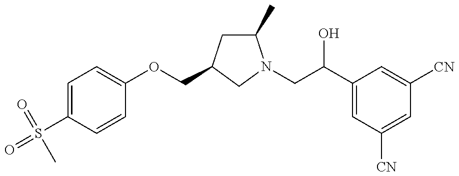 [SMILES: C[C@@H]1C[C@H](COc2ccc(S(C)(=O)=O)cc2)CN1CC(O)c1cc(C#N)cc(C#N)c1] | 5-[(1R or 1S)-1-hydroxy-2- [(2R,4S)-4-[(4- methanesulfonylphenoxy) methyl]-2-methylpyrrolidin-1- yl]ethyl]benzene-1,3- dicarbonitrile |
| 119 | 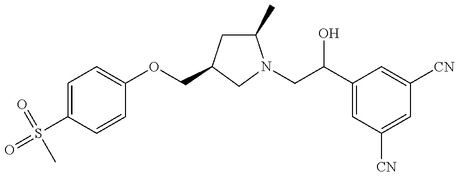 [SMILES: C[C@@H]1C[C@H](COc2ccc(S(C)(=O)=O)cc2)CN1CC(O)c1cc(C#N)cc(C#N)c1] | 5-[(1S or 1R)-1-hydroxy-2- [(2R,4S)-4-[(4- methanesulfonylphenoxy) methyl]-2-methylpyrrolidin-1- yl]ethyl]benzene-1,3- dicarbonitrile |
| 120 | 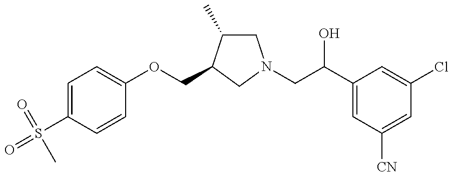 [SMILES: C[C@@H]1CN(CC(O)c2cc(Cl)cc(C#N)c2)C[C@H]1COc1ccc(S(C)(=O)=O)cc1] | 3-chloro-5-{1-hydroxy-2- [(3S,4S)-3-[(4- methanesulfonylphenoxy) methyl]-4-methylpyrrolidin-1- ylethyl}benzonitrile |
| 121 | 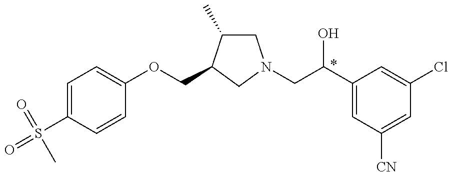 [SMILES: C[C@@H]1CN(CC(O)c2cc(Cl)cc(C#N)c2)C[C@H]1COc1ccc(S(C)(=O)=O)cc1] | 3-chloro-5-[(1S or 1R)-1- hydroxy-2-[(3S,4S)-3-[(4- methanesulfonylphenoxy) methyl]-4-methylpyrrolidin-1- yl]ethyl]benzonitrile |
| 122 | 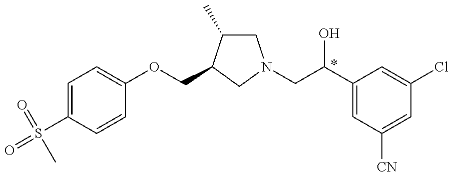 [SMILES: C[C@@H]1CN(CC(O)c2cc(Cl)cc(C#N)c2)C[C@H]1COc1ccc(S(C)(=O)=O)cc1] | 3-chloro-5-[(1R or 1S)-1- hydroxy-2-[(3S,4S)-3-[(4- methanesulfonylphenoxy) methyl]-4-methylpyrrolidin-1- yl]ethyl]benzonitrile |
| 123 | 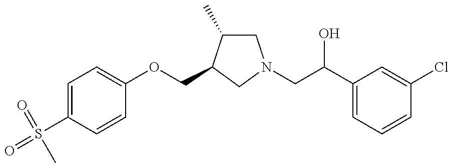 [SMILES: C[C@@H]1CN(CC(O)c2cccc(Cl)c2)C[C@H]1COc1ccc(S(C)(=O)=O)cc1] | 1-(3-chlorophenyl)-2- ((3S,4S)-3-methyl-4-((4- (methylsulfonyl)phenoxy) methyl)pyrrolidin-1-yl)ethan- 1-ol |
| 124 | 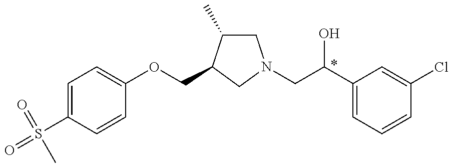 [SMILES: C[C@@H]1CN(CC(O)c2cccc(Cl)c2)C[C@H]1COc1ccc(S(C)(=O)=O)cc1] | (1R or 1S)-1-(3- chlorophenyl)-2-[(3S,4S)-3- [(4 methanesulfonylphenoxy) methyl]-4-methylpyrrolidin-1- yl]ethan-1-ol |
| 125 | 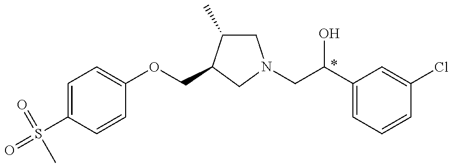 [SMILES: C[C@@H]1CN(CC(O)c2cccc(Cl)c2)C[C@H]1COc1ccc(S(C)(=O)=O)cc1] | (1S or 1R)-1-(3- chlorophenyl)-2-[(3S,4S)-3- [(4- methanesulfonylphenoxy) methyl]-4-methylpyrrolidin-1- yl]ethan-1-ol |
| 126 | 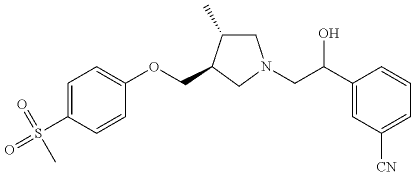 [SMILES: C[C@@H]1CN(CC(O)c2cccc(C#N)c2)C[C@H]1COc1ccc(S(C)(=O)=O)cc1] | 3-[1-hydroxy-2-[(3S,4S)-3- [(4- methanesulfonylphenoxy) methyl]-4-methylpyrrolidin-1- yl]ethyl]benzonitrile |
| 127 | 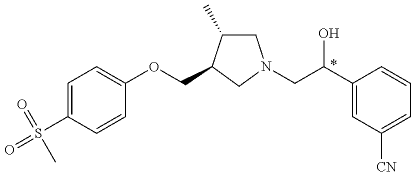 [SMILES: C[C@@H]1CN(CC(O)c2cccc(C#N)c2)C[C@H]1COc1ccc(S(C)(=O)=O)cc1] | 3-[(1S or 1R)-1-hydroxy-2- [(3S,4S)-3-[(4- methanesulfonylphenoxy) methyl]-4-methylpyrrolidin-1- yl]ethyl]benzonitrile |
| 128 | 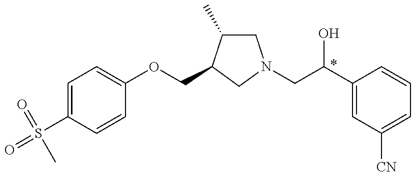 [SMILES: C[C@@H]1CN(CC(O)c2cccc(C#N)c2)C[C@H]1COc1ccc(S(C)(=O)=O)cc1] | 3-[(1R or 1S)-1-hydroxy-2- [(3S,4S)-3-[(4- methanesulfonylphenoxy) methyl]-4-methylpyrrolidin-1- yl]ethyl]benzonitrile |
| 129 | 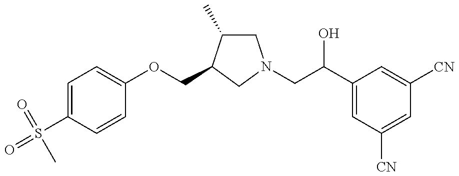 [SMILES: C[C@@H]1CN(CC(O)c2cc(C#N)cc(C#N)c2)C[C@H]1COc1ccc(S(C)(=O)=O)cc1] | 5-{1-hydroxy-2-[(3S,4S)-3- [(4- methanesulfonylphenoxy) methyl]-4-methylpyrrolidin-1- yl]ethyl}benzene-1,3- dicarbonitrile |
| 130 | 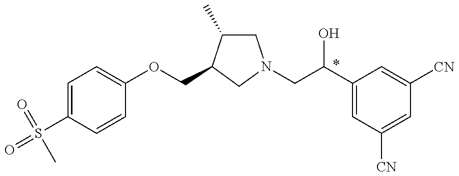 [SMILES: C[C@@H]1CN(CC(O)c2cc(C#N)cc(C#N)c2)C[C@H]1COc1ccc(S(C)(=O)=O)cc1] | 5-[(1R or 1S)-1-hydroxy-2- [(3S,4S)-3-[(4- methanesulfonylphenoxy) methyl]-4-methylpyrrolidin-1- yl]ethyl]benzene-1,3- dicarbonitrile |
| 131 | 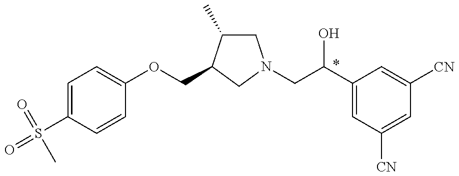 [SMILES: C[C@@H]1CN(CC(O)c2cc(C#N)cc(C#N)c2)C[C@H]1COc1ccc(S(C)(=O)=O)cc1] | 5-[(1S or 1R)-1-hydroxy-2- [(3S,4S)-3-[(4- methanesulfonylphenoxy) methyl]-4-methylpyrrolidin-1- yl]ethyl]benzene-1,3- dicarbonitrile |
| 132 | 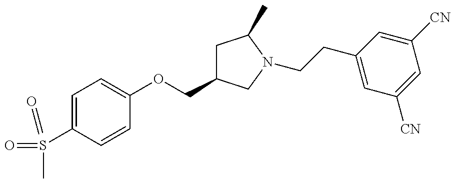 | 5-{2-[(2R,4S)-4-[(4- methanesulfonylphenoxy) methyl]-2-methylpyrrolidin- 1-ylethyl}benzene-1,3- dicarbonitrile |
| 133 | 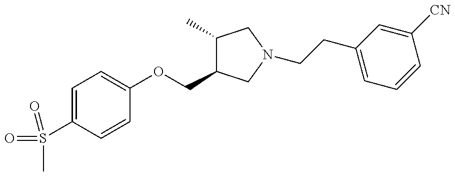 [SMILES: C[C@@H]1CN(CCc2cccc(C#N)c2)C[C@H]1COc1ccc(S(C)(=O)=O)cc1] | (3S,4S)-1-[2-(3- chlorophenyl)ethyl]-3-[(4- methanesulfonylphenoxy) methyl]-4-methylpyrrolidine |
| 134 | 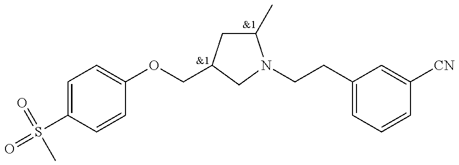 [SMILES: CC1CC(COc2ccc(S(C)(=O)=O)cc2)CN1CCc1cccc(C#N)c1] | (2S,5S)-1-[2-(3- chlorophenyl)ethyl]-5-[(4- methanesulfonylphenoxy) methyl]-2-methylpiperidine |
| 135 | 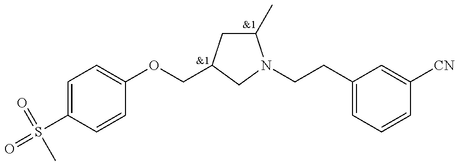 [SMILES: CC1CC(COc2ccc(S(C)(=O)=O)cc2)CN1CCc1cccc(C#N)c1] | rac-cis or trans-3-{2-[4-[(4- methanesulfonylphenoxy)m ethyl]-2-methylpyrrolidin-1- yl]ethyl}benzonitrile |
| 136 | 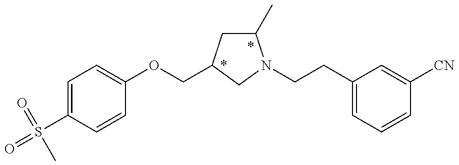 [SMILES: CC1CC(COc2ccc(S(C)(=O)=O)cc2)CN1CCc1cccc(C#N)c1] | rac-trans or cis-3-{2-[4-[(4- methanesulfonylphenoxy) methyl]-2-methylpyrrolidin-1- yl]ethyl}benzonitrile |
| 137 | 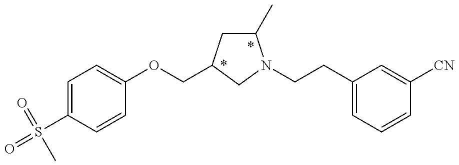 [SMILES: CC1CC(COc2ccc(S(C)(=O)=O)cc2)CN1CCc1cccc(C#N)c1] | 3-{2-[(25,4R or 2R,4S or 2R,4R or 25,4S)-4-[(4- methanesulfonylphenoxy) methyl]-2-methylpyrrolidin-1- ylethyl}benzonitrile |
| 138 | 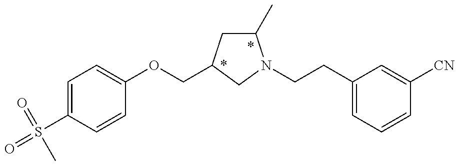 [SMILES: CC1CC(COc2ccc(S(C)(=O)=O)cc2)CN1CCc1cccc(C#N)c1] | 3-{2-[(2R,4S or 2S,4R or 2R,4R or 2S,4S)-4-[(4- methanesulfonylphenoxy) methyl]-2-methylpyrrolidin-1- yl]ethyl}benzonitrile |
| 139 | 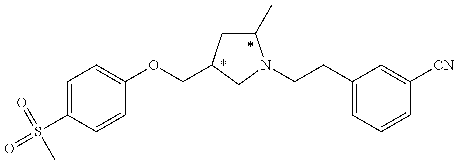 [SMILES: CC1CC(COc2ccc(S(C)(=O)=O)cc2)CN1CCc1cccc(C#N)c1] | 3-{2-[(2S,4S or 2S,4R or 2R,4S or 2R,4R)-4-[(4- methanesulfonylphenoxy) methyl]-2-methylpyrrolidin- 1-ylethyl}benzonitrile |
| 140 | 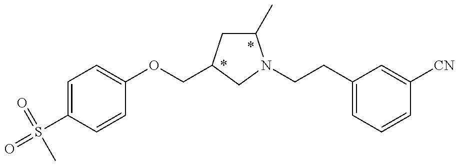 [SMILES: CC1CC(COc2ccc(S(C)(=O)=O)cc2)CN1CCc1cccc(C#N)c1] | 3-{2-[(2R,4R or 2S,4S or 2S,4R or 2R,4S)-4-[(4- methanesulfonylphenoxy) methyl]-2- methylpyrrolidin-1- yl]ethyl}benzonitrile |
| 141 | 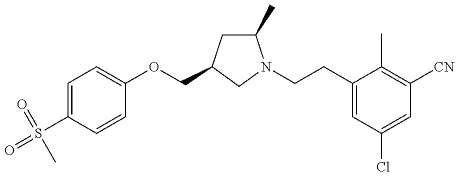 [SMILES: Cc1c(C#N)cc(Cl)cc1CCN1C[C@@H](COc2ccc(S(C)(=O)=O)cc2)C[C@H]1C] | 5-chloro-3-{2-[(2R,4S)-4- [(4- methanesulfonylphenoxy) methyl]-2-methylpyrrolidin- 1-yl]ethyl}-2- methylbenzonitrile |
| 142 | 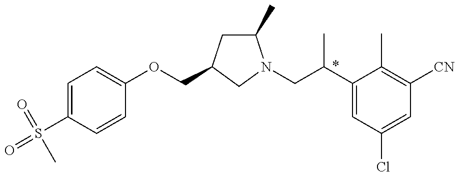 [SMILES: Cc1c(C#N)cc(Cl)cc1C(C)CN1C[C@@H](COc2ccc(S(C)(=O)=O)cc2)C[C@H]1C] | 3-chloro-5-[(2R or 2S)-1- [(2R,4S)-4-[(4- methanesulfonylphenoxy) methyl]-2-methylpyrrolidin-1- yl]propan-2-yl]benzonitrile |
| 143 | 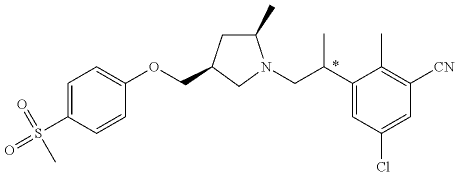 [SMILES: Cc1c(C#N)cc(Cl)cc1C(C)CN1C[C@@H](COc2ccc(S(C)(=O)=O)cc2)C[C@H]1C] | 3-chloro-5-[(2S or 2R)-1- [(2R,4S)-4-[(4- methanesulfonylphenoxy) methyl]-2- methylpyrrolidin-1- yl]propan-2-yl]benzonitrile |
| 144 | 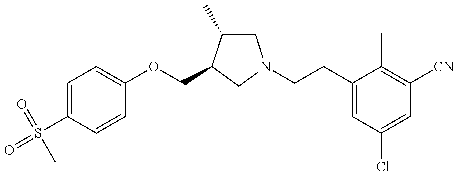 [SMILES: Cc1c(C#N)cc(Cl)cc1CCN1C[C@@H](COc2ccc(S(C)(=O)=O)cc2)[C@H](C)C1] | 5-chloro-3-{2-[(3S,4S)-3- [(4- methanesulfonylphenoxy) methyl]-4-methylpyrrolidin- 1-ylethyl}-2- methylbenzonitrile |
| 145 | 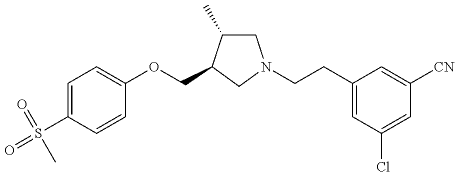 [SMILES: C[C@@H]1CN(CCc2cc(Cl)cc(C#N)c2)C[C@H]1COc1ccc(S(C)(=O)=O)cc1] | 5-{2-[(3S,4S)-3-[(4- methanesulfonylphenoxy) methyl]-4-methylpyrrolidin-1- ylethyl}benzene-1,3- dicarbonitrile |
| 146 | 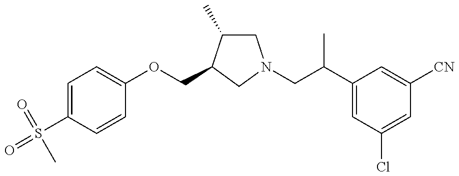 [SMILES: CC(CN1C[C@@H](COc2ccc(S(C)(=O)=O)cc2)[C@H](C)C1)c1cc(Cl)cc(C#N)c1] | 3-chloro-5-[1-[(3S,4S)-3-[(4- methanesulfonylphenoxy) methyl]-4-methylpyrrolidin-1- yl]propan-2-yl]benzonitrile |
| 147 | 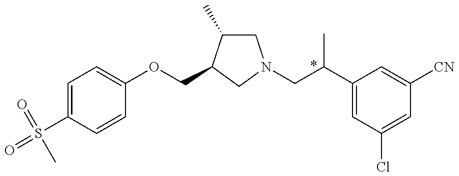 [SMILES: CC(CN1C[C@@H](COc2ccc(S(C)(=O)=O)cc2)[C@H](C)C1)c1cc(Cl)cc(C#N)c1] | 3-chloro-5-[(2R or 2S)-1- [(3S,4S)-3-[(4- methanesulfonylphenoxy) methyl]-4-methylpyrrolidin-1- yl]propan-2-yl]benzonitrile |
| 148 | 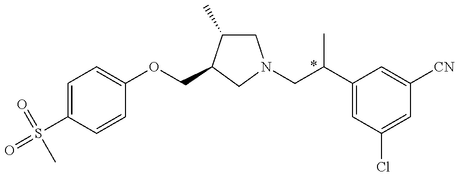 [SMILES: CC(CN1C[C@@H](COc2ccc(S(C)(=O)=O)cc2)[C@H](C)C1)c1cc(Cl)cc(C#N)c1] | 3-chloro-5-[(2S or 2R)-1- [(3S,4S)-3-[(4- methanesulfonylphenoxy) methyl]-4-methylpyrrolidin-1- yl]propan-2-yl]benzonitrile |
| 149 | 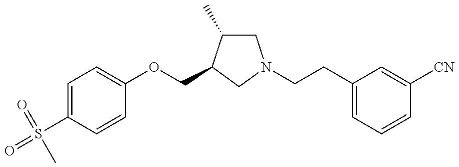 [SMILES: C[C@@H]1CN(CCc2cccc(C#N)c2)C[C@H]1COc1ccc(S(C)(=O)=O)cc1] | 3-{2-[(3S,4S)-3-[(4- methanesulfonylphenoxy) methyl]-4-methylpyrrolidin-1- yl]ethyl}benzonitrile |
| 150 | 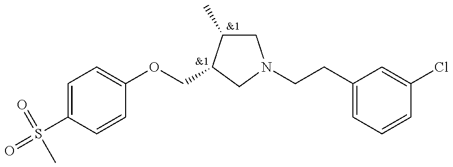 [SMILES: C[C@@H]1CN(CCc2cccc(Cl)c2)C[C@@H]1COc1ccc(S(C)(=O)=O)cc1] | rac-cis-1-[2-(3- chlorophenyl)ethyl]-3-[(4- methanesulfonylphenoxy) methyl]-4-methylpyrrolidine |
| 151 | 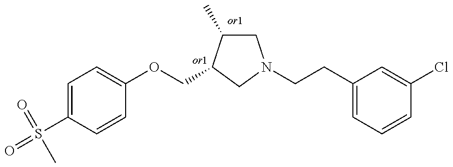 [SMILES: C[C@@H]1CN(CCc2cccc(Cl)c2)C[C@@H]1COc1ccc(S(C)(=O)=O)cc1] | (3R,4S or 3S,4R)-1-[2-(3- chlorophenyl)ethyl]-3-[(4- methanesulfonylphenoxy) methyl]-4-methylpyrrolidine |
| 152 | 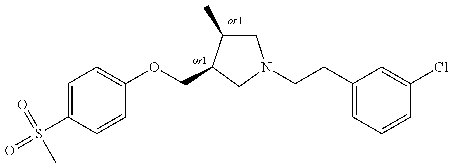 [SMILES: C[C@H]1CN(CCc2cccc(Cl)c2)C[C@H]1COc1ccc(S(C)(=O)=O)cc1] | (3S,4R or 3R,4S)-1-[2-(3- chlorophenyl)ethyl]-3-[(4- methanesulfonylphenoxy) methyl]-4-methylpyrrolidine |
| 153 | 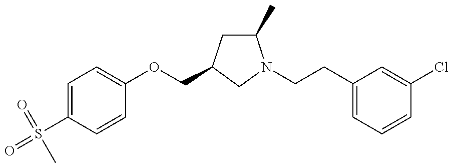 [SMILES: C[C@@H]1C[C@H](COc2ccc(S(C)(=O)=O)cc2)CN1CCc1cccc(Cl)c1] | (2R,4S)-1-[2-(3- chlorophenyl)ethyl]-4-[(4- methanesulfonylphenoxy) methyl]-2-methylpyrrolidine |
| 154 | 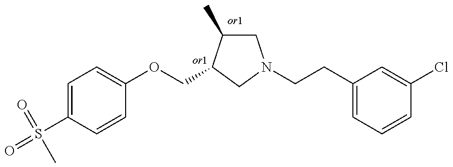 [SMILES: C[C@H]1CN(CCc2cccc(Cl)c2)C[C@@H]1COc1ccc(S(C)(=O)=O)cc1] | (3R,4R or 3S,4S)-1-[2-(3- chlorophenyl)ethyl]-3-[(4- methanesulfonylphenoxy) methyl]-4-methylpyrrolidine |
| 155 | 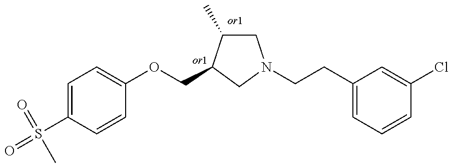 [SMILES: C[C@@H]1CN(CCc2cccc(Cl)c2)C[C@H]1COc1ccc(S(C)(=O)=O)cc1] | (3S,4S or 3R,4R)-1-[2-(3- chlorophenyl)ethyl]-3-[(4- methanesulfonylphenoxy) methyl]-4-methylpyrrolidine |
| 156 | 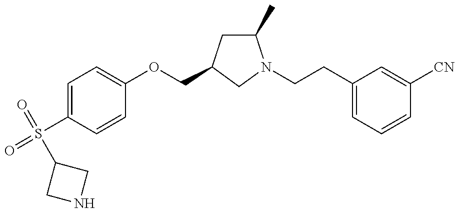 [SMILES: C[C@@H]1C[C@H](COc2ccc(S(=O)(=O)C3CNC3)cc2)CN1CCc1cccc(C#N)c1] | 3-{2-[(2R,4S)-4-{ [4- (azetidine-3- sulfonyl)phenoxy]methyl}- 2-methylpyrrolidin-1- yl]ethyl}benzonitrile |
| 157 | 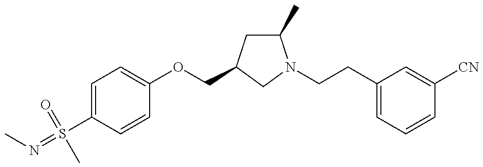 [SMILES: CN=S(C)(=O)c1ccc(OC[C@H]2C[C@@H](C)N(CCc3cccc(C#N)c3)C2)cc1] | 3-{2-[(2R,4S)-2-methyl-4- ({4- [methyl(methylimino)oxo- 26- sulfanyl]phenoxy} methyl)pyrrolidin-1- yl]ethyl}benzonitrile |
| 158 | 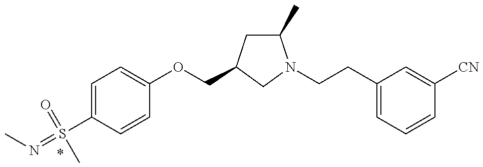 [SMILES: CN=S(C)(=O)c1ccc(OC[C@H]2C[C@@H](C)N(CCc3cccc(C#N)c3)C2)cc1] | 3-{2-[(2R,4S)-2-methyl-4- ({4-[(R or S)methyl(methylimino)oxo- 26- sulfanyl]phenoxy}methyl) pyrrolidin-1- yl]ethyl}benzonitrile |
| 159 | 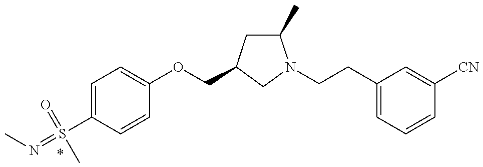 [SMILES: CN=S(C)(=O)c1ccc(OC[C@H]2C[C@@H](C)N(CCc3cccc(C#N)c3)C2)cc1] | 3-{2-[(2R,4S)-2-methyl-4- ({4-[(S or R)methyl (methylimino)oxo- 26- sulfanyl]phenoxy}methyl) pyrrolidin-1- yl]ethyl}benzonitrile |
| 160 | 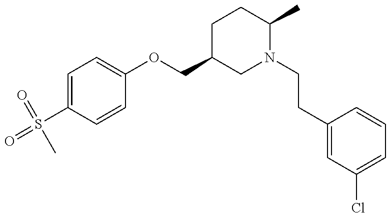 [SMILES: C[C@@H]1CC[C@H](COc2ccc(S(C)(=O)=O)cc2)CN1CCc1cccc(Cl)c1] | (2R,5S)-1-[2-(3- chlorophenyl)ethyl]-5-[(4- methanesulfonylphenoxy) methyl]-2-methylpiperidine |
| 161 | 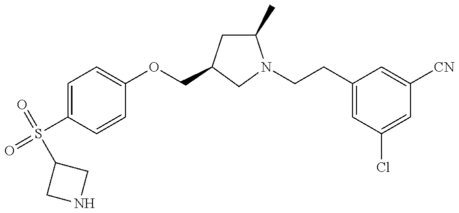 [SMILES: C[C@@H]1C[C@H](COc2ccc(S(=O)(=O)C3CNC3)cc2)CN1CCc1cc(Cl)cc(C#N)c1] | 3-{2-[(2R,4S)-4-{[4- (azetidine-3- sulfonyl)phenoxy]methyl}- 2-methylpyrrolidin-1- ylethyl}-5- chlorobenzonitrile |
| 162 | 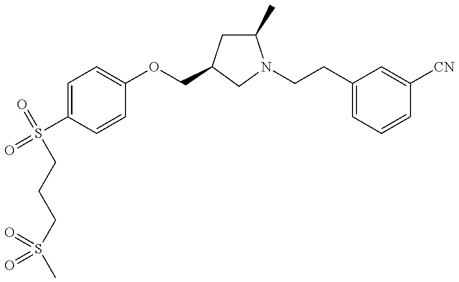 [SMILES: C[C@@H]1C[C@H](COc2ccc(S(=O)(=O)CCCS(C)(=O)=O)cc2)CN1CCc1cccc(C#N)c1] | 3-{2-[(2R,4S)-4-{ [4-(3- methanesulfonylpropanesulfonyl) phenoxy]methyl}-2- methylpyrrolidin-1- ylethyl}benzonitrile |
| 163 | 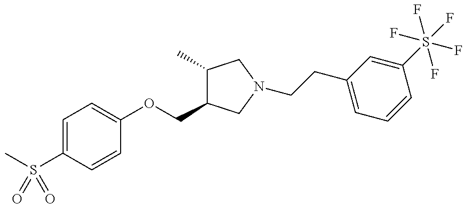 [SMILES: C[C@@H]1CN(CCc2cccc(S(F)(F)(F)(F)F)c2)C[C@H]1COc1ccc(S(C)(=O)=O)cc1] | (3S,4S)-3-[(4- methanesulfonylphenoxy) methyl]-4-methyl-1-{2-[3- (pentafluoro-26- sulfanyl)phenyl]ethyl} pyrrolidine |
| 164 | 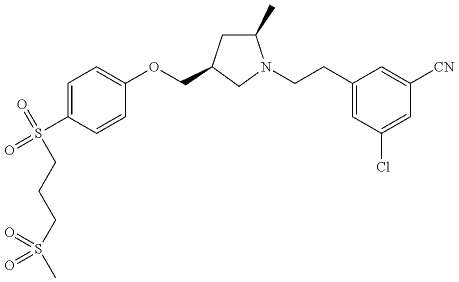 [SMILES: C[C@@H]1C[C@H](COc2ccc(S(=O)(=O)CCCS(C)(=O)=O)cc2)CN1CCc1cc(Cl)cc(C#N)c1] | 3-chloro-5-{2-[(2R,4S)-4- {[4-(3- methanesulfonylpropanesulfonyl) phenoxy]methyl}-2- methylpyrrolidin-1- yl]ethyl}benzonitrile |
| 165 | 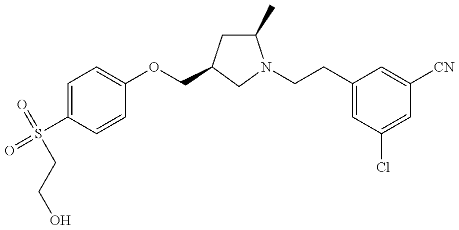 [SMILES: C[C@@H]1C[C@H](COc2ccc(S(=O)(=O)CCO)cc2)CN1CCc1cc(Cl)cc(C#N)c1] | 3-chloro-5-{2-[(2R,4S)-4- {[4-(2- hydroxyethanesulfonyl) phenoxy]methyl}-2- methylpyrrolidin-1- yl]ethyl}benzonitrile |
| 166 | 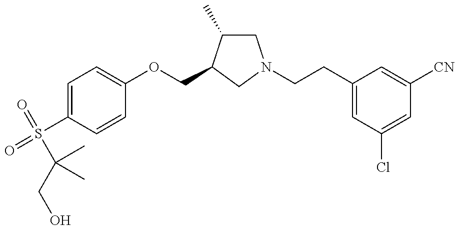 [SMILES: C[C@@H]1CN(CCc2cc(Cl)cc(C#N)c2)C[C@H]1COc1ccc(S(=O)(=O)C(C)(C)CO)cc1] | 3-chloro-5-{2-[(3S,4S)-3- {[4-(1-hydroxy-2- methylpropane-2- sulfonyl)phenoxy]methyl}- 4-methylpyrrolidin-1- yl]ethyl}benzonitrile |
| 167 | 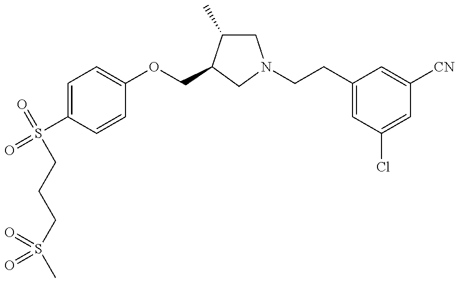 [SMILES: C[C@@H]1CN(CCc2cc(Cl)cc(C#N)c2)C[C@H]1COc1ccc(S(=O)(=O)CCCS(C)(=O)=O)cc1] | 3-chloro-5-{2-[(3S,4S)-3- {[4-(3- methanesulfonylpropanesulfonyl) phenoxy]methyl}-4- methylpyrrolidin-1- yl]ethyl}benzonitrile |
| 168 | 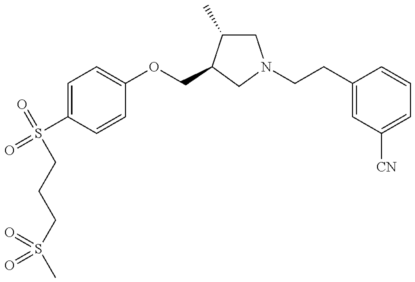 [SMILES: C[C@@H]1CN(CCc2cccc(C#N)c2)C[C@H]1COc1ccc(S(=O)(=O)CCCS(C)(=O)=O)cc1] | 3-{2-[(3S,4S)-3-{ [4-(3- methanesulfonylpropanesulfonyl) phenoxy]methyl}-4- methylpyrrolidin-1- yl]ethyl}benzonitrile |
| 169 | 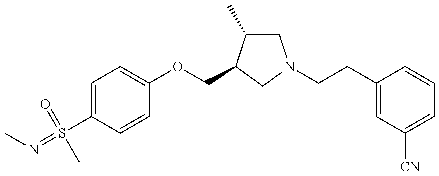 [SMILES: CN=S(C)(=O)c1ccc(OC[C@@H]2CN(CCc3cccc(C#N)c3)C[C@H]2C)cc1] | 3-{2-[(3S,4S)-3-methyl-4- ({4- [methyl(methylimino)oxo- 26- sulfanyl]phenoxy}methyl) pyrrolidin-1- yl]ethyl}benzonitrile |
| 170 | 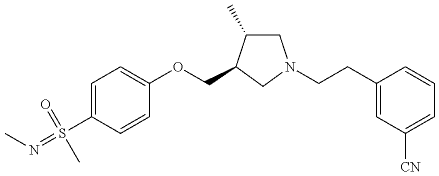 [SMILES: CN=S(C)(=O)c1ccc(OC[C@@H]2CN(CCc3cccc(C#N)c3)C[C@H]2C)cc1] | 3-{2-[(3S,4S)-3-methyl-4- ({4-[(R or S)- methyl(methylimino)oxo- 26- sulfanyl]phenoxy}methyl) pyrrolidin-1- ylethyl}benzonitrile |
| 171 | 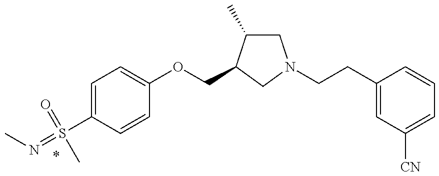 [SMILES: CN=S(C)(=O)c1ccc(OC[C@@H]2CN(CCc3cccc(C#N)c3)C[C@H]2C)cc1] | 3-{2-[(3S,4S)-3-methyl-4- ({4-[(S or R)- methyl(methylimino)oxo- 26- sulfanyl]phenoxy }methyl) pyrrolidin-1- yl]ethyl}benzonitrile |
| 172 | 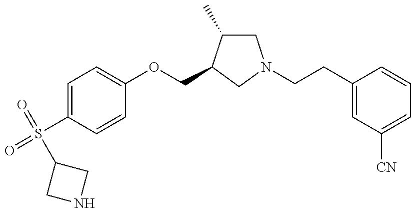 [SMILES: C[C@@H]1CN(CCc2cccc(C#N)c2)C[C@H]1COc1ccc(S(=O)(=O)C2CNC2)cc1] | 3-{2-[(3S,4S)-3-{[4- (azetidine-3- sulfonyl)phenoxy]methyl}- 4-methylpyrrolidin-1- yl]ethyl}benzonitrile |
| 173 | 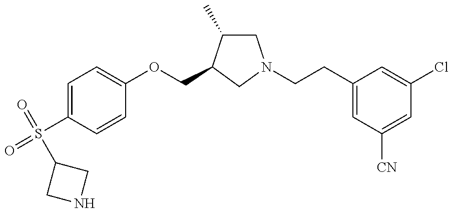 [SMILES: C[C@@H]1CN(CCc2cc(Cl)cc(C#N)c2)C[C@H]1COc1ccc(S(=O)(=O)C2CNC2)cc1] | 3-{2-[(3S,4S)-3-{[4- (azetidine-3- sulfonyl)phenoxy]methyl}- 4-methylpyrrolidin-1- yl]ethyl}-5- chlorobenzonitrile |
| 174 | 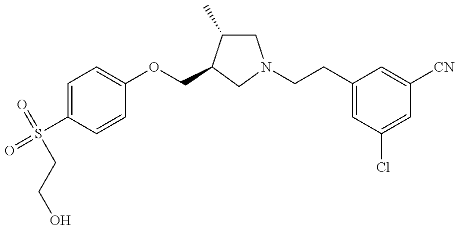 [SMILES: C[C@@H]1CN(CCc2cc(Cl)cc(C#N)c2)C[C@H]1COc1ccc(S(=O)(=O)CCO)cc1] | 3-chloro-5-{2-[(3S,4S)-3- {[4-(2- hydroxyethanesulfonyl) phenoxy]methyl}-4- methylpyrrolidin-1- yl]ethyl}benzonitrile |
| 175 | 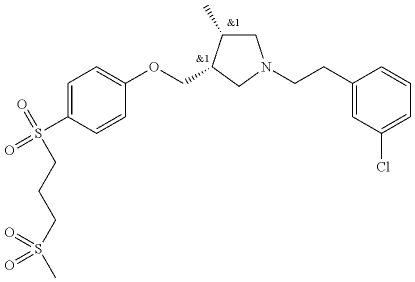 [SMILES: C[C@@H]1CN(CCc2cccc(Cl)c2)C[C@@H]1COc1ccc(S(=O)(=O)CCCS(C)(=O)=O)cc1] | rac-trans-1-[2-(3- chlorophenyl)ethyl]-3-{[4-(3- methanesulfonylpropanesulfonyl) phenoxy]methyl}-4- methylpyrrolidine |
| 176 | 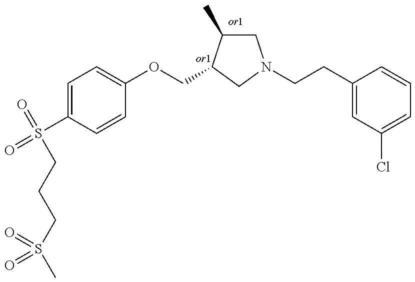 [SMILES: C[C@H]1CN(CCc2cccc(Cl)c2)C[C@@H]1COc1ccc(S(=O)(=O)CCCS(C)(=O)=O)cc1] | (3R,4R or 3S,4S)-1-[2-(3- chlorophenyl)ethyl]-3-{[4-(3- methanesulfonylpropanesulfonyl) phenoxy]methyl}-4- methylpyrrolidine |
| 177 | 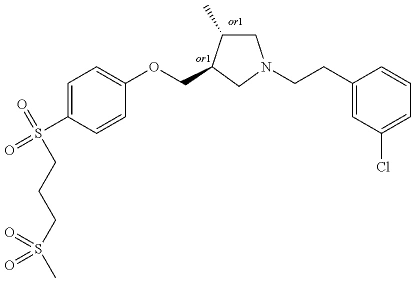 [SMILES: C[C@@H]1CN(CCc2cccc(Cl)c2)C[C@H]1COc1ccc(S(=O)(=O)CCCS(C)(=O)=O)cc1] | (3S,4S or 3R,4R)-1-[2-(3- chlorophenyl)ethyl]-3-{[4-(3- methanesulfonylpropanesulfonyl) phenoxy]methyl}-4- methylpyrrolidine |
| 181 | 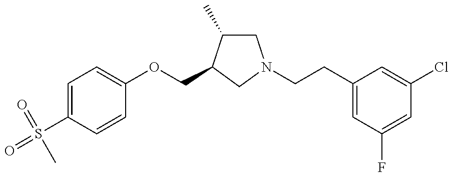 [SMILES: C[C@@H]1CN(CCc2cc(F)cc(Cl)c2)C[C@H]1COc1ccc(S(C)(=O)=O)cc1] | (3S,4S)-1-[2-(3-chloro-5- fluorophenyl)ethyl]-3-[(4- methanesulfonylphenoxy) methyl]-4- methylpyrrolidine |
| 182 | 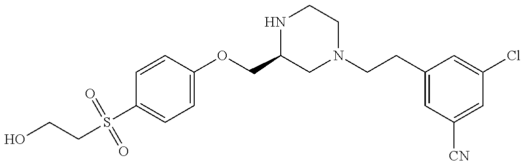 [SMILES: N#Cc1cc(Cl)cc(CCN2CCN[C@H](COc3ccc(S(=O)(=O)CCO)cc3)C2)c1] | 3-chloro-5-{2-[(3S)-3-{[4-(2- hydroxyethanesulfonyl) phenoxy] methyl} piperazin-1- yl]ethyl}benzonitrile |
| 183 | 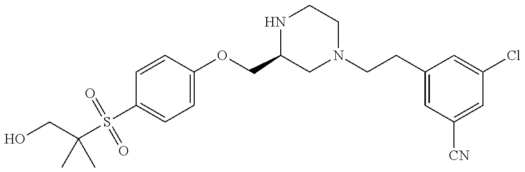 [SMILES: CC(C)(CO)S(=O)(=O)c1ccc(OC[C@@H]2CN(CCc3cc(Cl)cc(C#N)c3)CCN2)cc1] | 3-chloro-5-{2-[(3S)-3-{[4- (1-hydroxy-2- methylpropane-2- sulfonyl)phenoxy]methyl} piperazin-1- yl]ethyl}benzonitrile |
| 184 | 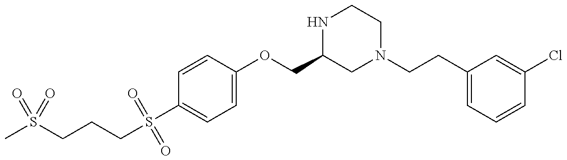 [SMILES: CS(=O)(=O)CCCS(=O)(=O)c1ccc(OC[C@@H]2CN(CCc3cccc(Cl)c3)CCN2)cc1] | (3S)-1-[2-(3- chlorophenyl)ethyl]-3-{[4-(3- methanesulfonylpropanesulfonyl) phenoxy]methyl}piperazine |
| 185 | 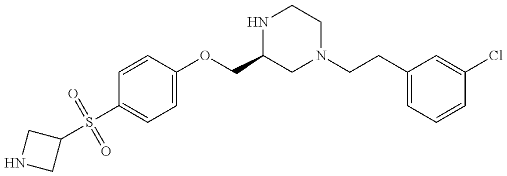 [SMILES: O=S(=O)(c1ccc(OC[C@@H]2CN(CCc3cccc(Cl)c3)CCN2)cc1)C1CNC1] | (3S)-3-{[4-(azetidine-3- sulfonyl)phenoxy]methyl}- 1-[2-(3- chlorophenyl)ethyl] piperazine |
| 186 | 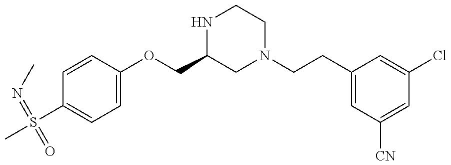 [SMILES: CN=S(C)(=O)c1ccc(OC[C@@H]2CN(CCc3cc(Cl)cc(C#N)c3)CCN2)cc1] | 3-chloro-5-{2-[(3S)-3-({4- [methyl(methylimino)oxo- 26- sulfanyl]phenoxy} methyl)piperazin-1- yl]ethyl}benzonitrile |
| 187 | 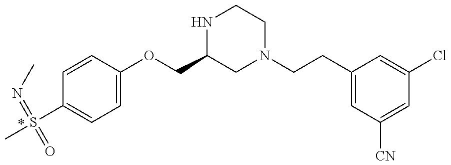 [SMILES: CN=S(C)(=O)c1ccc(OC[C@@H]2CN(CCc3cc(Cl)cc(C#N)c3)CCN2)cc1] | 3-chloro-5-{2-[(3S)-3-({4- [(S or R)- methyl(methylimino)oxo- 26- sulfanyl]phenoxy}methyl) piperazin-1- ylethyl}benzonitrile |
| 188 | 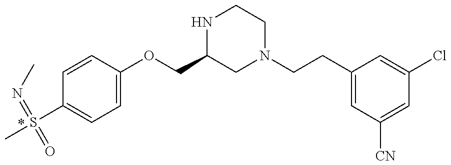 [SMILES: CN=S(C)(=O)c1ccc(OC[C@@H]2CN(CCc3cc(Cl)cc(C#N)c3)CCN2)cc1] | 3-chloro-5-{2-[(3S)-3-({4- [(R or S)- methyl(methylimino)oxo- 26- sulfanyl]phenoxy}methyl) piperazin-1- yl]ethyl}benzonitrile |
| 189 | 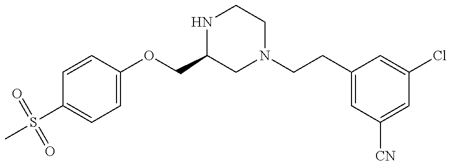 [SMILES: CS(=O)(=O)c1ccc(OC[C@@H]2CN(CCc3cc(Cl)cc(C#N)c3)CCN2)cc1] | 3-chloro-5-{2-[(3S)-3-[(4- methanesulfonylphenoxy) methyl]piperazin-1- yl]ethyl}benzonitrile |
| 190 | 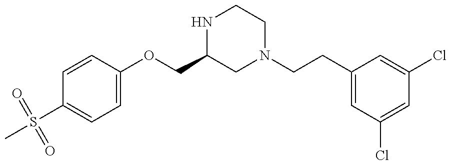 [SMILES: CS(=O)(=O)c1ccc(OC[C@@H]2CN(CCc3cc(Cl)cc(Cl)c3)CCN2)cc1] | (3S)-1-[2-(3,5- dichlorophenyl)ethyl]-3-[(4- methanesulfonylphenoxy) methyl]piperazine |
| 191 | 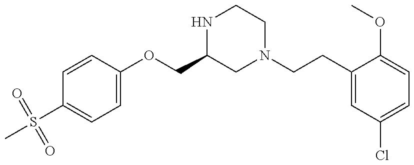 [SMILES: COc1ccc(Cl)cc1CCN1CCN[C@H](COc2ccc(S(C)(=O)=O)cc2)C1] | (3S)-1-[2-(5-chloro-2- methoxyphenyl)ethyl]-3- [(4- methanesulfonylphenoxy) methyl]piperazine |
| 192 | 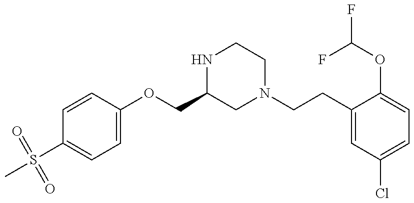 [SMILES: CS(=O)(=O)c1ccc(OC[C@@H]2CN(CCc3cc(Cl)ccc3OC(F)F)CCN2)cc1] | (3S)-1-{2-[5-chloro-2- (difluoromethoxy)phenyl] ethyl}-3-[(4- methanesulfonylphenoxy) methyl]piperazine |
| 193 | 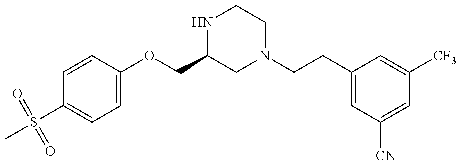 [SMILES: CS(=O)(=O)c1ccc(OC[C@@H]2CN(CCc3cc(C#N)cc(C(F)(F)F)c3)CCN2)cc1] | 3-{2-[(3S)-3-[(4- methanesulfonylphenoxy) methyl]piperazin-1-yl]ethyl}- 5- (trifluoromethyl)benzonitrile |
| 194 | 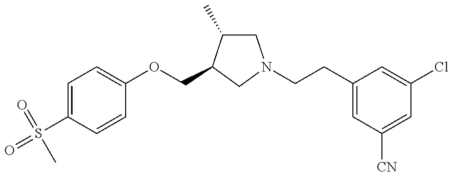 [SMILES: C[C@@H]1CN(CCc2cc(Cl)cc(C#N)c2)C[C@H]1COc1ccc(S(C)(=O)=O)cc1] | 3-chloro-5-{2-[(3S,4S)-3- [(4- methanesulfonylphenoxy) methyl]-4-methylpyrrolidin-1- yl]ethyl}benzonitrile |
| 195 | 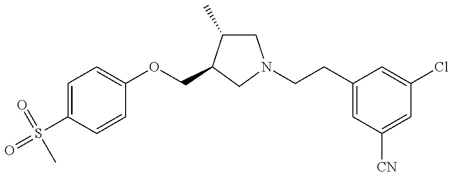 [SMILES: C[C@@H]1CN(CCc2cc(Cl)cc(C#N)c2)C[C@H]1COc1ccc(S(C)(=O)=O)cc1] | 3-fluoro-5-{2-[(3S,4S)-3- [(4- methanesulfonylphenoxy) methyl]-4-methylpyrrolidin-1- ylethyl}benzonitrile |
| 196 | 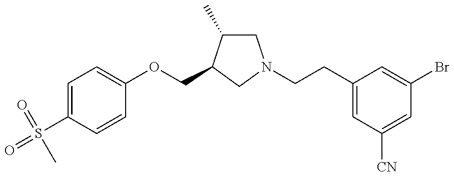 [SMILES: C[C@@H]1CN(CCc2cc(Br)cc(C#N)c2)C[C@H]1COc1ccc(S(C)(=O)=O)cc1] | 3-bromo-5-{2-[(3S,4S)-3- [(4- methanesulfonylphenoxy) methyl]-4-methylpyrrolidin-1- yl]ethyl}benzonitrile |
| 197 | 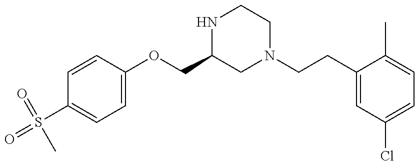 [SMILES: Cc1ccc(Cl)cc1CCN1CCN[C@H](COc2ccc(S(C)(=O)=O)cc2)C1] | (3S)-1-[2-(5-chloro-2- methylphenyl)ethyl]-3-[(4- methanesulfonylphenoxy) methyl]piperazine |
| 198 | 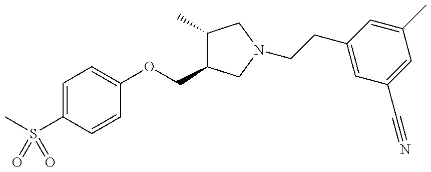 | 3-{2-[(3S,4S)-3-[(4- methanesulfonylphenoxy) methyl]-4-methylpyrrolidin-1- yl]ethyl}-5- methylbenzonitrile |
| 199 | 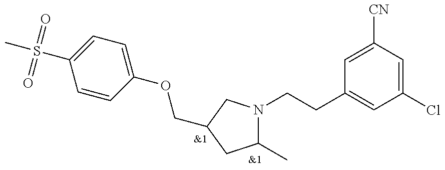 [SMILES: CC1CC(COc2ccc(S(C)(=O)=O)cc2)CN1CCc1cc(Cl)cc(C#N)c1] | rac-cis or trans-3-chloro-5- {2-[4-[(4- methanesulfonylphenoxy) methyl]-2-methylpyrrolidin-1- ylethyl}benzonitrile |
| 200 | 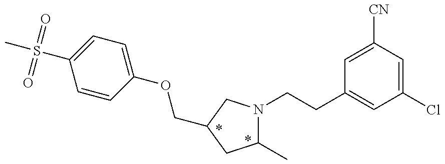 [SMILES: CC1CC(COc2ccc(S(C)(=O)=O)cc2)CN1CCc1cc(Cl)cc(C#N)c1] | 3-chloro-5-{2-[(2R,4S or 2S,4R or 2R,4R or 2S,4S)-4- [(4- methanesulfonylphenoxy) methyl]-2-methylpyrrolidin-1- yl]ethyl}benzonitrile |
| 201 | 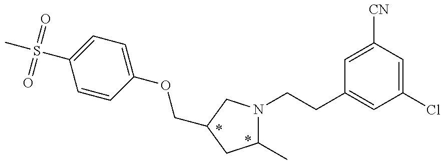 [SMILES: CC1CC(COc2ccc(S(C)(=O)=O)cc2)CN1CCc1cc(Cl)cc(C#N)c1] | 3-chloro-5-{2-[(2S,4R or 2R,4S or 2R,4R or 2S,4S)-4- [(4- methanesulfonylphenoxy) methyl]-2-methylpyrrolidin-1- ylethyl}benzonitrile |
| 202 | 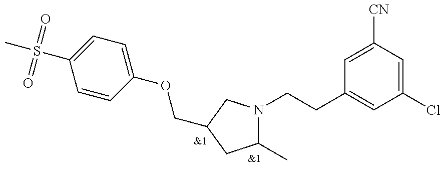 [SMILES: CC1CC(COc2ccc(S(C)(=O)=O)cc2)CN1CCc1cc(Cl)cc(C#N)c1] | rac-trans or cis-3-chloro-5- {2-[4-[(4- methanesulfonylphenoxy) methyl]-2-methylpyrrolidin-1- yl]ethyl}benzonitrile |
| 203 | 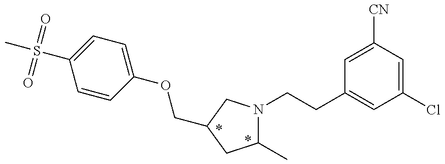 [SMILES: CC1CC(COc2ccc(S(C)(=O)=O)cc2)CN1CCc1cc(Cl)cc(C#N)c1] | 3-chloro-5-(2-((2R,4S or 2S,4R or 2R,4R or 2S,4S)-2- methyl-4-((4- (methylsulfonyl)phenoxy) methyl)pyrrolidin-1- yl)ethyl)benzonitrile |
| 204 | 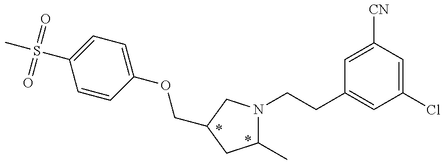 [SMILES: CC1CC(COc2ccc(S(C)(=O)=O)cc2)CN1CCc1cc(Cl)cc(C#N)c1] | 3-chloro-5-(2-((2R,4S or 2S,4R or 2S,4S or 2R.4R)-2- methyl-4-((4- (methylsulfonyl)phenoxy) methyl)pyrrolidin-1- yl)ethyl)benzonitrile |
| 205 | 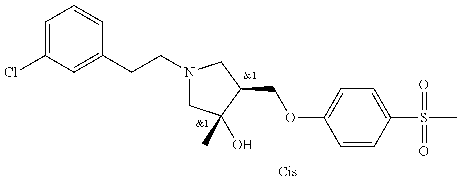 [SMILES: C[C@@]1(O)CN(CCc2cccc(Cl)c2)C[C@H]1COc1ccc(S(C)(=O)=O)cc1] | rac-cis-1-[2-(3- chlorophenyl)ethyl]-4-[(4- methanesulfonylphenoxy) methyl]-3-methylpyrrolidin-3- ol |
| 206 | 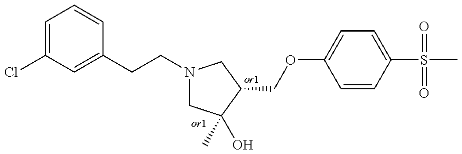 [SMILES: C[C@]1(O)CN(CCc2cccc(Cl)c2)C[C@@H]1COc1ccc(S(C)(=O)=O)cc1] | (3R,4R or 3S,4S)-1-[2-(3- chlorophenyl)ethyl]-4-[(4- methanesulfonylphenoxy) methyl]-3-methylpyrrolidin-3- ol |
| 207 | 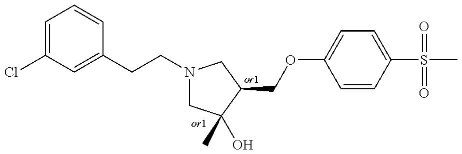 [SMILES: C[C@@]1(O)CN(CCc2cccc(Cl)c2)C[C@H]1COc1ccc(S(C)(=O)=O)cc1] | (3S,4S or 3R,4R)-1-(3- chlorophenethyl)-3-methyl- 4-((4- (methylsulfonyl)phenoxy) methyl)pyrrolidin-3-ol |
| 208 | 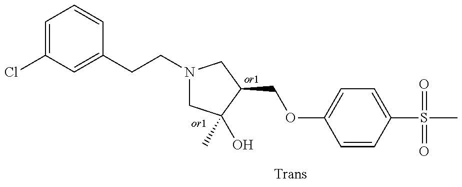 [SMILES: C[C@]1(O)CN(CCc2cccc(Cl)c2)C[C@H]1COc1ccc(S(C)(=O)=O)cc1] | rac-trans-1-[2-(3- chlorophenyl)ethyl]-4-[(4- methanesulfonylphenoxy) methyl]-3-methylpyrrolidin-3- ol |
| 209 | 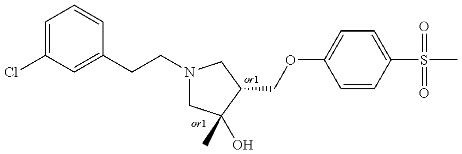 [SMILES: C[C@@]1(O)CN(CCc2cccc(Cl)c2)C[C@@H]1COc1ccc(S(C)(=O)=O)cc1] | (3R,4S or 3S,4R)-1-[2-(3- chlorophenyl)ethyl]-4-[(4- methanesulfonylphenoxy) methyl]-3-methylpyrrolidin-3- ol |
| 210 | 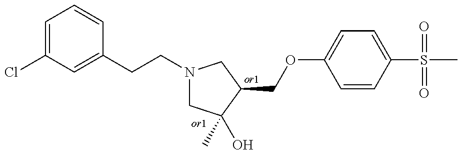 [SMILES: C[C@]1(O)CN(CCc2cccc(Cl)c2)C[C@H]1COc1ccc(S(C)(=O)=O)cc1] | (3S,4R or 3R,4S)-1-[2-(3- chlorophenyl)ethyl]-4-[(4- methanesulfonylphenoxy) methyl]-3-methylpyrrolidin-3- ol |
| 211 | 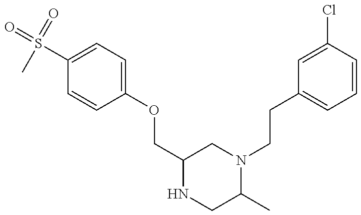 [SMILES: CC1CNC(COc2ccc(S(C)(=O)=O)cc2)CN1CCc1cccc(Cl)c1] | 1-[2-(3-chlorophenyl)ethyl]- 5-[(4- methanesulfonylphenoxy) methyl]-2-methylpiperazine |
| 212 | 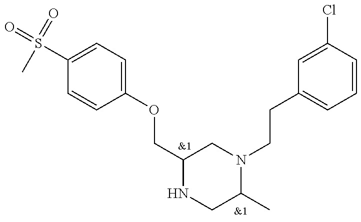 [SMILES: CC1CNC(COc2ccc(S(C)(=O)=O)cc2)CN1CCc1cccc(Cl)c1] | rac-cis or trans-1-[2-(3- chlorophenyl)ethyl]-5-[(4- methanesulfonylphenoxy) methyl]-2-methylpiperazine |
| 213 | 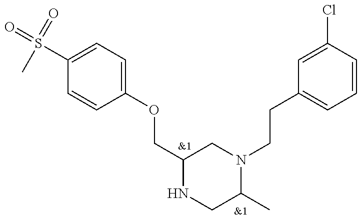 [SMILES: CC1CNC(COc2ccc(S(C)(=O)=O)cc2)CN1CCc1cccc(Cl)c1] | rac-trans or cis-1-[2-(3- chlorophenyl)ethyl]-5-[(4- methanesulfonylphenoxy) methyl]-2-methylpiperazine |
| 214 | 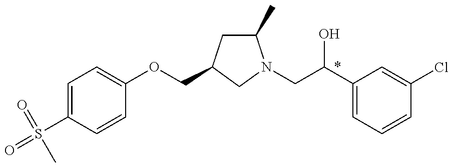 [SMILES: C[C@@H]1C[C@H](COc2ccc(S(C)(=O)=O)cc2)CN1CC(O)c1cccc(Cl)c1] | (1R) or (1S)-1-(3- chlorophenyl)-2-[(2R,4S)-4- [(4- methanesulfonylphenoxy) methyl]-2-methylpyrrolidin-1- yl]ethan-1-ol |
| 215 | 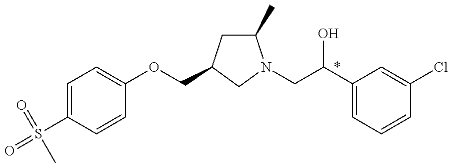 [SMILES: C[C@@H]1C[C@H](COc2ccc(S(C)(=O)=O)cc2)CN1CC(O)c1cccc(Cl)c1] | (1S) or (1R)-1-(3- chlorophenyl)-2-[(2R,4S)-4- [(4- methanesulfonylphenoxy) methyl]-2-methylpyrrolidin-1- yllethan-1-ol |

In some embodiments, a compound of formula (A) or (A′) is selected from the group consisting of:
1-(3-chlorophenethyl)-3-((4-(methylsulfonyl)phenoxy)methyl)azepane;
1-(3-chlorophenethyl)-3-((4-(methylsulfonyl)phenoxy)methyl)piperidin-3-ol;
N-(4-((1-(3-chlorophenethyl)piperidin-3-yl)methoxy)phenyl)-N-methylmethanesulfonamide;
N-(4-((4-(3-chlorophenethyl)-1,4-oxazepan-2-yl)methoxy)phenyl)-N-methylmethanesulfonamide;
1-(3-chlorophenethyl)-3-((4-(methylsulfonyl)phenoxy)methyl)piperazine;
1-(3-chlorophenethyl)-3-((4-(methylsulfonyl)phenoxy)methyl)piperidine;
1-(3-chlorophenyl)-2-(3-((4-(methylsulfonyl)phenoxy)methyl)piperidin-1-yl)ethan-1-ol;
1-(3-chlorophenyl)-2-(3-((4-(methylsulfonyl)phenoxy)methyl)piperidin-1-yl)ethanol;
1-(2-(3-chlorophenyl)-2-methoxyethyl)-3-((4-(methylsulfonyl)phenoxy)methyl)piperidine;
1-(3-chlorophenethyl)-3-((4-(methylsulfonyl)phenoxy)methyl)piperidin-4-ol;
1-(3-chlorophenethyl)-4-methoxy-3-((4-(methylsulfonyl)phenoxy) methyl)piperidine;
1-(3-chlorophenethyl)-3-(4-(methylsulfonyl)phenethyl)piperidine;
1-(3-chlorophenethyl)-2,2-dimethyl-4-((4-(methylsulfonyl)phenoxy)methyl)pyrrolidine;
N-(4-(1-(3-chlorophenethyl)-4-methylpyrrolidin-3-yl)methoxy)phenyl)-N-methylmethanesulfonamide;
N-(4-(3-((3-chlorophenethyl)amino)cyclobutoxy)phenyl)-N-methylmethanesulfonamide;
N-(4-((1-(3-chlorophenethyl)pyrrolidin-3-yl)methoxy)phenyl)-N-methylmethanesulfonamide;
1-(3-chlorophenethyl)-3-((4-(methylsulfonyl)phenoxy)methyl)pyrrolidine;
1-(3-chlorophenethyl)-3-methyl-4-((4-(methylsulfonyl)phenoxy)methyl)pyrrolidine;
1-(3-chlorophenethyl)-2-methyl-4-((4-(methylsulfonyl)phenoxy)methyl)pyrrolidine;
N-(4-(3-((3-chlorophenethyl)(cyclopropylmethyl)amino)-2-hydroxypropoxy)phenyl)-N-methylmethanesulfonamide;
N-(4-(3-((3-chlorophenethyl)(ethyl)amino)-2-hydroxypropoxy)phenyl)-N-methylmethanesulfonamide;
N-(4-(3-((3-chlorophenethyl)amino)-2-methylpropoxy)phenyl)-N-methylmethanesulfonamide;
1-(2-(3-chlorobenzyl)piperidin-1-yl)-3-(4-(methylsulfonyl)phenoxy)propan-2-ol;
N-(4-(3-(3-(3-chlorophenyl)cyclobutyl)amino)-2-hydroxypropoxy)phenyl)-N-methylmethanesulfonamide;
1-(3-(3-chlorophenyl)cyclobutyl)amino)-3-(4-(methylsulfonyl)phenoxy)propan-2-ol;
N-(4-((2)-3-(3-(3-chlorophenyl)-2,2-dimethylpyrrolidin-1-yl)-2-hydroxypropoxy)phenyl)-N-methylmethanesulfonamide;
N-(4-((2)-3-(3-(3-chlorophenyl)pyrrolidin-1-yl)-2-hydroxypropoxy)phenyl)-N-methylmethanesulfonamide;
N-(4-(3-(3-chlorophenyl)pyrrolidin-1-yl)-2-hydroxypropoxy)phenyl)-N-methylmethanesulfonamide;
N-(4-(3-(2-(3-chlorophenyl)morpholino)-2-hydroxypropoxy)phenyl)-N-methylmethanesulfonamide;
N-(4-(3-((3-chlorophenethyl)amino)-2-hydroxypropoxy)phenyl)-N-methylmethanesulfonamide;
N-(4-(3-((1-(3-chlorophenyl)propan-2-yl)amino)-2-hydroxypropoxy)phenyl)-N-methylmethanesulfonamide;
N-(4-(3-((2,5-dichlorophenethyl)amino)-2-hydroxypropoxy)phenyl)-N-methylmethanesulfonamide;
N-(4-(3-((3,5-dichlorophenethyl)amino)-2-hydroxypropoxy)phenyl)-N-methylmethanesulfonamide;
N-(4-(3-((3-(3-chlorobenzyl)tetrahydrofuran-3-yl)amino)-2-hydroxypropoxy)phenyl)-N-methylmethanesulfonamide;
1-((3-chlorophenethyl)amino)-3-(4-(methylsulfonyl)phenoxy)propan-2-ol;
1-((1-(3-chlorophenyl)-2-methylpropan-2-yl)amino)-3-(4-(methylsulfonyl)phenoxy)propan-2-ol;
N-(4-(2-hydroxy-3-((2-methoxyphenethyl)amino)propoxy)phenyl)-N-methylmethanesulfonamide;
N-(4-(3-((3-cyanophenethyl)amino)-2-hydroxypropoxy)phenyl)-N-methylmethanesulfonamide;
N-(4-(3-((2-ethylphenethyl)amino)-2-hydroxypropoxy)phenyl)-N-methylmethanesulfonamide;
N-(4-(3-((2-(difluoromethoxy)phenethyl)amino)-2-hydroxypropoxy)phenyl)-N-methylmethanesulfonamide;
1-((2-(3-chlorophenyl)-1-cyclopropylethyl)amino)-3-(4-(methylsulfonyl)phenoxy)propan-2-ol;
N-(4-(3-((3-chlorophenethyl)amino)-2-hydroxy-2-methylpropoxy)phenyl)-N-methylmethanesulfonamide;
N-(4-((3-((3-chlorophenethyl)amino)-2-hydroxypropyl)amino)phenyl)-N-methylmethanesulfonamide;
1-[2-(3-chlorophenyl)ethyl]-3-{[4-(2-methoxyethanesulfonyl)phenoxy]methyl}-4-methylpyrrolidine;
1-[2-(3-chlorophenyl)ethyl]-3-{[4-(2-methoxyethanesulfonyl)phenoxy]methyl}-4-methylpyrrolidine;
1-[2-(3-chlorophenyl)ethyl]-3-{[4-(2-methoxyethanesulfonyl)phenoxy]methyl}-4-methylpyrrolidine;
1-[2-(3-chlorophenyl)ethyl]-4-[(4-methanesulfonylphenoxy)methyl]-3-methylpyrrolidin-3-ol;
1-[2-(3-chlorophenyl)ethyl]-4-[(4-methanesulfonylphenoxy)methyl]-3-methylpyrrolidin-3-ol;
1-[2-(3-chlorophenyl)ethyl]-5-[(4-methanesulfonylphenoxy)methyl]-2-methylpiperidine;
1-[2-(3-chlorophenyl)ethyl]-3-[(4-methanesulfonylphenoxy)methyl]-4-methylpyrrolidine;
1-[2-(3-chlorophenyl)ethyl]-3-[(4-methanesulfonylphenoxy)methyl]-4-methylpyrrolidine;
1-[2-(3-chlorophenyl)ethyl]-3-[(4-methanesulfonylphenoxy)methyl]-4-methylpyrrolidine;
1-[2-(3-chlorophenyl)ethyl]-4-[(4-methanesulfonylphenoxy)methyl]-3-methylpyrrolidin-3-ol;
2-[4-({1-[2-(3-chlorophenyl)ethyl]-4-methylpyrrolidin-3-yl}methoxy)benzenesulfonyl]ethan-1-ol;
1-[2-(3-chlorophenyl)ethyl]-4-[(4-methanesulfonylphenoxy)methyl]-3-methylpyrrolidin-3-ol;
2-[4-({1-[2-(3-chlorophenyl)ethyl]-4-methylpyrrolidin-3-yl}methoxy)benzenesulfonyl]ethan-1-ol;
1-[2-(3-chlorophenyl)ethyl]-4-[(4-methanesulfonylphenoxy)methyl]-3-methylpyrrolidin-3-ol;
1-[2-(3-chlorophenyl)ethyl]-4-[(4-methanesulfonylphenoxy)methyl]-3-methylpyrrolidin-3-ol;
1-[2-(3-chlorophenyl)ethyl]-5-[(4-methanesulfonylphenoxy)methyl]-2-methylpiperazine;
1-[2-(3-chlorophenyl)ethyl]-3-[(4-methanesulfonylphenoxy)methyl]-4-methylpyrrolidine;
1-[2-(3-chlorophenyl)ethyl]-5-[(4-methanesulfonylphenoxy)methyl]-2-methylpiperazine;
1-[2-(3-chlorophenyl)ethyl]-5-[(4-methanesulfonylphenoxy)methyl]-2-methylpiperazine;
1-[2-(3-chlorophenyl)ethyl]-3-[(4-methanesulfonylphenoxy)methyl]-4-methylpyrrolidine;
1-[2-(3-chlorophenyl)ethyl]-3-[(4-methanesulfonylphenoxy)methyl]-4-methylpyrrolidine;
1-[2-(3-chlorophenyl)ethyl]-4-[(4-methanesulfonylphenoxy)methyl]-2-methylpyrrolidine;
1-[2-(3,5-dichlorophenyl)ethyl]-3-[(4-methanesulfonylphenoxy)methyl]piperazine;
1-[2-(5-chloro-2-methylphenyl)ethyl]-3-[(4-methanesulfonylphenoxy)methyl]piperazine;
1-[2-(3-chlorophenyl)ethyl]-3-{[4-(3-methanesulfonylpropanesulfonyl)phenoxy]methyl}piperazine;
3-(2-{3-[(4-methanesulfonylphenoxy)methyl]-4-methylpyrrolidin-1-yl}ethyl)benzonitrile;
3-(2-{4-[(4-methanesulfonylphenoxy)methyl]-2-methylpyrrolidin-1-yl}ethyl)benzonitrile;
3-(2-{4-[(4-methanesulfonylphenoxy)methyl]-2-methylpyrrolidin-1-yl}ethyl)benzonitrile;
3-(2-{4-[(4-methanesulfonylphenoxy)methyl]-2-methylpyrrolidin-1-yl}ethyl)benzonitrile;
1-[2-(3-chlorophenyl)ethyl]-3-{[4-(3-methanesulfonylpropanesulfonyl)phenoxy]methyl}-4-methylpyrrolidine;
3-(2-{4-[(4-methanesulfonylphenoxy)methyl]-2-methylpyrrolidin-1-yl}ethyl)benzonitrile;
3-(2-{4-[(4-methanesulfonylphenoxy)methyl]-2-methylpyrrolidin-1-yl}ethyl)benzonitrile;
3-(2-{4-[(4-methanesulfonylphenoxy)methyl]-2-methylpyrrolidin-1-yl}ethyl)benzonitrile;
1-[2-(3-chlorophenyl)ethyl]-5-[(4-methanesulfonylphenoxy)methyl]-2-methylpiperidine;
3-chloro-5-(1-hydroxy-2-{3-[(4-methanesulfonylphenoxy)methyl]-4-methylpyrrolidin-1-yl}ethyl)benzonitrile;
1-(3-chlorophenyl)-2-{3-[(4-methanesulfonylphenoxy)methyl]-4-methylpyrrolidin-1-yl}ethan-1-ol;
1-[2-(3-chlorophenyl)ethyl]-3-{[4-(3-methanesulfonylpropanesulfonyl)phenoxy]methyl}-4-methylpyrrolidine;
3-chloro-5-(2-{3-[(4-methanesulfonylphenoxy)methyl]piperazin-1-yl}ethyl)benzonitrile;
3-chloro-5-(2-{3-[(4-methanesulfonylphenoxy)methyl]-4-methylpyrrolidin-1-yl}ethyl)benzonitrile;
1-[2-(3-chlorophenyl)ethyl]-3-{[4-(3-methanesulfonylpropanesulfonyl)phenoxy]methyl}-4-methylpyrrolidine;
3-chloro-5-(1-hydroxy-2-{3-[(4-methanesulfonylphenoxy)methyl]-4-methylpyrrolidin-1-yl}ethyl)benzonitrile;
3-chloro-5-(1-hydroxy-2-{3-[(4-methanesulfonylphenoxy)methyl]-4-methylpyrrolidin-1-yl}ethyl)benzonitrile;
1-(3-chlorophenyl)-2-{3-[(4-methanesulfonylphenoxy)methyl]-4-methylpyrrolidin-1-yl}ethan-1-ol;
1-(3-chlorophenyl)-2-{3-[(4-methanesulfonylphenoxy)methyl]-4-methylpyrrolidin-1-yl}ethan-1-ol;
3-chloro-5-(2-{4-[(4-methanesulfonylphenoxy)methyl]-2-methylpyrrolidin-1-yl}ethyl)benzonitrile;
3-chloro-5-(1-hydroxy-2-{4-[(4-methanesulfonylphenoxy)methyl]-2-methylpyrrolidin-1-yl}ethyl)benzonitrile;
3-chloro-5-(1-hydroxy-2-{4-[(4-methanesulfonylphenoxy)methyl]-2-methylpyrrolidin-1-yl}ethyl)benzonitrile;
3-chloro-5-(2-{4-[(4-methanesulfonylphenoxy)methyl]-2-methylpyrrolidin-1-yl}ethyl)benzonitrile;
3-{[4-(azetidine-3-sulfonyl)phenoxy]methyl}-1-[2-(3-chlorophenyl)ethyl]piperazine;
3-chloro-5-[2-(4-{[4-(3-methanesulfonylpropanesulfonyl)phenoxy]methyl}-2-methylpyrrolidin-1-yl)ethyl]benzonitrile;
3-chloro-5-[2-(4-{[4-(2-hydroxyethanesulfonyl)phenoxy]methyl}-2-methylpyrrolidin-1-yl)ethyl]benzonitrile;
3-chloro-5-[2-(3-{[4-(2-hydroxyethanesulfonyl)phenoxy]methyl}-4-methylpyrrolidin-1-yl)ethyl]benzonitrile;
3-fluoro-5-(2-{3-[(4-methanesulfonylphenoxy)methyl]-4-methylpyrrolidin-1-yl}ethyl)benzonitrile;
3-[(4-methanesulfonylphenoxy)methyl]-4-methyl-1-{2-[3-(pentafluoro-lambda6-sulfanyl)phenyl]ethyl}pyrrolidine;
3-chloro-5-[2-(3-{[4-(1-hydroxy-2-methylpropane-2-sulfonyl)phenoxy]methyl}-4-methylpyrrolidin-1-yl)ethyl]benzonitrile;
3-chloro-5-(2-{4-[(4-methanesulfonylphenoxy)methyl]-2-methylpyrrolidin-1-yl}ethyl)benzonitrile;
3-chloro-5-(2-{4-[(4-methanesulfonylphenoxy)methyl]-2-methylpyrrolidin-1-yl}ethyl)benzonitrile;
1-[2-(5-chloro-2-methoxyphenyl)ethyl]-3-[(4-methanesulfonylphenoxy)methyl]piperazine;
3-chloro-5-(2-{4-[(4-methanesulfonylphenoxy)methyl]-2-methylpyrrolidin-1-yl}ethyl)benzonitrile;
3-chloro-5-(2-{4-[(4-methanesulfonylphenoxy)methyl]-2-methylpyrrolidin-1-yl}ethyl)benzonitrile;
1-{2-[5-chloro-2-(difluoromethoxy)phenyl]ethyl}-3-[(4-methanesulfonylphenoxy)methyl]piperazine;
1-(3-chlorophenyl)-2-{4-[(4-methanesulfonylphenoxy)methyl]-2-methylpyrrolidin-1-yl}ethan-1-ol;
1-(3-chlorophenyl)-2-{4-[(4-methanesulfonylphenoxy)methyl]-2-methylpyrrolidin-1-yl}ethan-1-ol;
3-chloro-5-[2-(3-{[4-(2-methanesulfonylethanesulfonyl)phenoxy]methyl}-4-methylpyrrolidin-1-yl)ethyl]benzonitrile;
1-[2-(3-chloro-5-fluorophenyl)ethyl]-3-[(4-methanesulfonylphenoxy)methyl]-4-methylpyrrolidine;
3-[2-(4-{[4-(3-methanesulfonylpropanesulfonyl)phenoxy]methyl}-2-methylpyrrolidin-1-yl)ethyl]benzonitrile;
3-chloro-5-(1-{4-[(4-methanesulfonylphenoxy)methyl]-2-methylpyrrolidin-1-yl}propan-2-yl)benzonitrile;
3-[2-(3-{[4-(3-methanesulfonylpropanesulfonyl)phenoxy]methyl}-4-methylpyrrolidin-1-yl)ethyl]benzonitrile;
3-chloro-5-[2-(3-{[4-(3-methanesulfonylpropanesulfonyl)phenoxy]methyl}-4-methylpyrrolidin-1-yl)ethyl]benzonitrile;
3-chloro-5-(1-{3-[(4-methanesulfonylphenoxy)methyl]-4-methylpyrrolidin-1-yl}propan-2-yl)benzonitrile;
3-chloro-5-(1-{4-[(4-methanesulfonylphenoxy)methyl]-2-methylpyrrolidin-1-yl}propan-2-yl)benzonitrile;
1-[2-(3-chlorophenyl)ethyl]-3-{[4-(2-methanesulfonylethanesulfonyl)phenoxy]methyl}-4-methylpyrrolidine;
3-chloro-5-(1-{3-[(4-methanesulfonylphenoxy)methyl]-4-methylpyrrolidin-1-yl}propan-2-yl)benzonitrile;
5-chloro-3-(2-{3-[(4-methanesulfonylphenoxy)methyl]-4-methylpyrrolidin-1-yl}ethyl)-2-methylbenzonitrile;
5-chloro-3-(2-{4-[(4-methanesulfonylphenoxy)methyl]-2-methylpyrrolidin-1-yl}ethyl)-2-methylbenzonitrile;
3-(1-hydroxy-2-{4-[(4-methanesulfonylphenoxy)methyl]-2-methylpyrrolidin-1-yl}ethyl)benzonitrile;
3-chloro-5-(1-{3-[(4-methanesulfonylphenoxy)methyl]-4-methylpyrrolidin-1-yl}propan-2-yl)benzonitrile;
3-chloro-5-[2-(3-{[4-(1-hydroxy-2-methylpropane-2-sulfonyl)phenoxy]methyl}piperazin-1-yl)ethyl]benzonitrile;
3-(2-{3-[(4-methanesulfonylphenoxy)methyl]piperazin-1-yl}ethyl)-5-(trifluoromethyl)benzonitrile;
3-(2-{3-[(4-methanesulfonylphenoxy)methyl]-4-methylpyrrolidin-1-yl}ethyl)-5-methylbenzonitrile;
3-[2-(3-{[4-(azetidine-3-sulfonyl)phenoxy]methyl}-4-methylpyrrolidin-1-yl)ethyl]benzonitrile;
3-chloro-5-[2-(3-{[4-(2-hydroxyethanesulfonyl)phenoxy]methyl}piperazin-1-yl)ethyl]benzonitrile;
5-(2-{4-[(4-methanesulfonylphenoxy)methyl]-2-methylpyrrolidin-1-yl}ethyl)benzene-1,3-dicarbonitrile;
5-(2-{3-[(4-methanesulfonylphenoxy)methyl]-4-methylpyrrolidin-1-yl}ethyl)benzene-1,3-dicarbonitrile;
3-[2-(4-{[4-(azetidine-3-sulfonyl)phenoxy]methyl}-2-methylpyrrolidin-1-yl)ethyl]-5-chlorobenzonitrile;
3-[2-(3-{[4-(azetidine-3-sulfonyl)phenoxy]methyl}-4-methylpyrrolidin-1-yl)ethyl]-5-chlorobenzonitrile;
3-bromo-5-(2-{3-[(4-methanesulfonylphenoxy)methyl]-4-methylpyrrolidin-1-yl}ethyl)benzonitrile;
3-[2-(4-{[4-(azetidine-3-sulfonyl)phenoxy]methyl}-2-methylpyrrolidin-1-yl)ethyl]benzonitrile;
3-(1-hydroxy-2-{4-[(4-methanesulfonylphenoxy)methyl]-2-methylpyrrolidin-1-yl}ethyl)benzonitrile;
3-{2-[3-methyl-4-({4-[methyl(methylimino)oxo-lambda6-sulfanyl]phenoxy}methyl)pyrrolidin-1-yl]ethyl}benzonitrile;
3-(1-hydroxy-2-{3-[(4-methanesulfonylphenoxy)methyl]-4-methylpyrrolidin-1-yl}ethyl)benzonitrile;
3-(1-hydroxy-2-{3-[(4-methanesulfonylphenoxy)methyl]-4-methylpyrrolidin-1-yl}ethyl)benzonitrile;
3-(1-hydroxy-2-{3-[(4-methanesulfonylphenoxy)methyl]-4-methylpyrrolidin-1-yl}ethyl)benzonitrile;
3-{2-[3-methyl-4-({4-[methyl(methylimino)oxo-lambda6-sulfanyl]phenoxy}methyl)pyrrolidin-1-yl]ethyl}benzonitrile;
3-{2-[3-methyl-4-({4-[methyl(methylimino)oxo-lambda6-sulfanyl]phenoxy}methyl)pyrrolidin-1-yl]ethyl}benzonitrile;
3-{2-[2-methyl-4-({4-[methyl(methylimino)oxo-lambda6-sulfanyl]phenoxy}methyl)pyrrolidin-1-yl]ethyl}benzonitrile;
5-(1-hydroxy-2-{3-[(4-methanesulfonylphenoxy)methyl]-4-methylpyrrolidin-1-yl}ethyl)benzene-1,3-dicarbonitrile;
5-(1-hydroxy-2-{3-[(4-methanesulfonylphenoxy)methyl]-4-methylpyrrolidin-1-yl}ethyl)benzene-1,3-dicarbonitrile;
5-(1-hydroxy-2-{4-[(4-methanesulfonylphenoxy)methyl]-2-methylpyrrolidin-1-yl}ethyl)benzene-1,3-dicarbonitrile;
5-(1-hydroxy-2-{4-[(4-methanesulfonylphenoxy)methyl]-2-methylpyrrolidin-1-yl}ethyl)benzene-1,3-dicarbonitrile;
3-{2-[2-methyl-4-({4-[methyl(methylimino)oxo-lambda6-sulfanyl]phenoxy}methyl)pyrrolidin-1-yl]ethyl}benzonitrile;
3-{2-[2-methyl-4-({4-[methyl(methylimino)oxo-lambda6-sulfanyl]phenoxy}methyl)pyrrolidin-1-yl]ethyl}benzonitrile;
5-(1-hydroxy-2-{3-[(4-methanesulfonylphenoxy)methyl]-4-methylpyrrolidin-1-yl}ethyl)benzene-1,3-dicarbonitrile;
3-chloro-5-{2-[3-({4-[methyl(methylimino)oxo-lambda6-sulfanyl]phenoxy}methyl)piperazin-1-yl]ethyl}benzonitrile;
3-chloro-5-{2-[3-({4-[methyl(methylimino)oxo-lambda6-sulfanyl]phenoxy}methyl)piperazin-1-yl]ethyl}benzonitrile; and
3-chloro-5-{2-[3-({4-[methyl(methylimino)oxo-lambda6-sulfanyl]phenoxy}methyl)piperazin-1-yl]ethyl}benzonitrile,
   or a stereoisomer or tautomer thereof, or a pharmaceutically acceptable salt of any of the foregoing. Isotopically labeled forms of any of the foregoing are also embraced, such as deuterated or tritiated forms (wherein at least one hydrogen is replaced by at least one deuterium or tritium) of any of the specific compounds detailed herein. Mixtures of any of the foregoing are also embraced and described. Prodrugs of any of the foregoing are also embraced herein.

In some embodiments, a compound of formula (A) or (A′) is selected from the group consisting of:
1-(3-chlorophenethyl)-3-((4-(methylsulfonyl)phenoxy)methyl)azepane;
(R)-1-(3-chlorophenethyl)-3-((4-(methylsulfonyl)phenoxy)methyl)azepane;
(S)-1-(3-chlorophenethyl)-3-((4-(methylsulfonyl)phenoxy)methyl)azepane;
1-(3-chlorophenethyl)-3-((4-(methylsulfonyl)phenoxy)methyl)piperidin-3-ol;
(R)-1-(3-chlorophenethyl)-3-((4-(methylsulfonyl)phenoxy)methyl)piperidin-3-ol;
(S)-1-(3-chlorophenethyl)-3-((4-(methylsulfonyl)phenoxy)methyl)piperidin-3-ol;
N-(4-((1-(3-chlorophenethyl)piperidin-3-yl)methoxy)phenyl)-N-methylmethanesulfonamide;
(S)—N-(4-((1-(3-chlorophenethyl)piperidin-3-yl)methoxy)phenyl)-N-methylmethanesulfonamide;
(R)—N-(4-((1-(3-chlorophenethyl)piperidin-3-yl)methoxy)phenyl)-N-methylmethanesulfonamide;
N-(4-((4-(3-chlorophenethyl)-1,4-oxazepan-2-yl)methoxy)phenyl)-N-methylmethanesulfonamide;
(R)—N-(4-((4-(3-chlorophenethyl)-1,4-oxazepan-2-yl)methoxy)phenyl)-N-methylmethanesulfonamide;
(S)—N-(4-((4-(3-chlorophenethyl)-1,4-oxazepan-2-yl)methoxy)phenyl)-N-methylmethanesulfonamide;
(S)-1-(3-chlorophenethyl)-3-((4-(methylsulfonyl)phenoxy)methyl)piperazine;
(R)-1-(3-chlorophenethyl)-3-((4-(methylsulfonyl)phenoxy)methyl)piperazine;
(R)-1-(3-chlorophenethyl)-3-((4-(methylsulfonyl)phenoxy)methyl)piperidine;
(S)-1-(3-chlorophenethyl)-3-((4-(methylsulfonyl)phenoxy)methyl)piperidine;
1-(3-chlorophenyl)-2-((S)-3-((4-(methylsulfonyl)phenoxy)methyl)piperidin-1-yl)ethan-1-ol;
(R)-1-(3-chlorophenyl)-2-((S)-3-((4-(methylsulfonyl)phenoxy)methyl)piperidin-1-yl)ethan-1-ol;
(S)-1-(3-chlorophenyl)-2-((S)-3-((4-(methylsulfonyl)phenoxy)methyl)piperidin-1-yl)ethan-1-ol;
1-(3-chlorophenyl)-2-((R)-3-((4-(methylsulfonyl)phenoxy)methyl)piperidin-1-yl)ethanol;
(R)-1-(3-chlorophenyl)-2-((R)-3-((4-(methylsulfonyl)phenoxy)methyl)piperidin-1-yl)ethanol;
(S)-1-(3-chlorophenyl)-2-((R)-3-((4-(methylsulfonyl)phenoxy)methyl)piperidin-1-yl)ethanol;
(S)-1-((R)-2-(3-chlorophenyl)-2-methoxyethyl)-3-((4-(methylsulfonyl)phenoxy)methyl)piperidine;
(S)-1-((S)-2-(3-chlorophenyl)-2-methoxyethyl)-3-((4-(methylsulfonyl)phenoxy)methyl)piperidine;
(R)-1-((R)-2-(3-chlorophenyl)-2-methoxyethyl)-3-((4-(methylsulfonyl)phenoxy)methyl)piperidine;
(R)-1-((S)-2-(3-chlorophenyl)-2-methoxyethyl)-3-((4-(methylsulfonyl)phenoxy)methyl)piperidine;
rac-trans or cis-1-(3-chlorophenethyl)-3-((4-(methylsulfonyl)phenoxy)methyl)piperidin-4-ol;
rac-cis or trans-1-(3-chlorophenethyl)-3-((4-(methylsulfonyl)phenoxy)methyl)piperidin-4-ol;
(3S,4R)-1-(3-chlorophenethyl)-3-((4-(methylsulfonyl)phenoxy)methyl)piperidin-4-ol;
(3R,4R)-1-(3-chlorophenethyl)-3-((4-(methylsulfonyl)phenoxy)methyl)piperidin-4-ol;
(3R,4S)-1-(3-chlorophenethyl)-3-((4-(methylsulfonyl)phenoxy)methyl)piperidin-4-ol;
(3S,4S)-1-(3-chlorophenethyl)-3-((4-(methylsulfonyl)phenoxy)methyl)piperidin-4-ol;
rac-trans-1-(3-chlorophenethyl)-4-methoxy-3-((4-(methylsulfonyl)phenoxy) methyl)piperidine;
rac-cis-1-(3-chlorophenethyl)-4-methoxy-3-((4-(methylsulfonyl)phenoxy) methyl)piperidine;
(3S,4R)-1-(3-chlorophenethyl)-4-methoxy-3-((4-(methylsulfonyl)phenoxy)methyl)piperidine;
(3R,4R)-1-(3-chlorophenethyl)-4-methoxy-3-((4-(methylsulfonyl)phenoxy)methyl)piperidine;
(3R,4S)-1-(3-chlorophenethyl)-4-methoxy-3-((4-(methylsulfonyl)phenoxy)methyl)piperidine;
(3S,4S)-1-(3-chlorophenethyl)-4-methoxy-3-((4-(methylsulfonyl)phenoxy)methyl)piperidine;
1-(3-chlorophenethyl)-3-(4-(methylsulfonyl)phenethyl)piperidine;
(S)-1-(3-chlorophenethyl)-3-(4-(methylsulfonyl)phenethyl)piperidine;
(R)-1-(3-chlorophenethyl)-3-(4-(methylsulfonyl)phenethyl)piperidine;
1-(3-chlorophenethyl)-2,2-dimethyl-4-((4-(methylsulfonyl)phenoxy) methyl) pyrrolidine;
(S)-1-(3-chlorophenethyl)-2,2-dimethyl-4-((4-(methylsulfonyl)phenoxy) methyl) pyrrolidine;
(R)-1-(3-chlorophenethyl)-2,2-dimethyl-4-((4-(methylsulfonyl)phenoxy)methyl)pyrrolidine;
rac-trans-N-(4-((1-(3-chlorophenethyl)-4-methylpyrrolidin-3-yl)methoxy)phenyl)-N-methylmethanesulfonamide;
N-(4-(((3S,4S)-1-(3-chlorophenethyl)-4-methylpyrrolidin-3-yl)methoxy)phenyl)-N-methylmethanesulfonamide;
N-(4-(((3R,4R)-1-(3-chlorophenethyl)-4-methylpyrrolidin-3-yl)methoxy)phenyl)-N-methylmethanesulfonamide;
trans-N-(4-(3-((3-chlorophenethyl)amino)cyclobutoxy)phenyl)-N-methylmethanesulfonamide;
(S)—N-(4-((1-(3-chlorophenethyl)pyrrolidin-3-yl)methoxy)phenyl)-N-methylmethanesulfonamide;
(R)—N-(4-((1-(3-chlorophenethyl)pyrrolidin-3-yl)methoxy)phenyl)-N-methylmethanesulfonamide;
(S)-1-(3-chlorophenethyl)-3-((4-(methylsulfonyl)phenoxy)methyl)pyrrolidine;
rac-trans-1-(3-chlorophenethyl)-3-methyl-4-((4-(methylsulfonyl)phenoxy)methyl)pyrrolidine;
rac-trans-1-(3-chlorophenethyl)-2-methyl-4-((4-(methylsulfonyl)phenoxy)methyl)pyrrolidine;
rac-cis-1-(3-chlorophenethyl)-2-methyl-4-((4-(methylsulfonyl)phenoxy)methyl)pyrrolidine;
(2R,4S)-1-(3-chlorophenethyl)-2-methyl-4-((4-(methylsulfonyl)phenoxy)methyl)pyrrolidine;
(2R,4R)-1-(3-chlorophenethyl)-2-methyl-4-((4-(methylsulfonyl)phenoxy)methyl)pyrrolidine;
(2S,4R)-1-(3-chlorophenethyl)-2-methyl-4-((4-(methylsulfonyl)phenoxy)methyl)pyrrolidine;
(2S,4S)-1-(3-chlorophenethyl)-2-methyl-4-((4-(methylsulfonyl)phenoxy)methyl)pyrrolidine;
(S)—N-(4-(3-((3-chlorophenethyl)(cyclopropylmethyl)amino)-2-hydroxypropoxy)phenyl)-N-methylmethanesulfonamide;
(S)—N-(4-(3-((3-chlorophenethyl)(ethyl)amino)-2-hydroxypropoxy)phenyl)-N-methylmethanesulfonamide;
(R)—N-(4-(3-((3-chlorophenethyl)amino)-2-methylpropoxy)phenyl)-N-methylmethanesulfonamide;
(2S)-1-(2-(3-chlorobenzyl)piperidin-1-yl)-3-(4-(methylsulfonyl)phenoxy)propan-2-ol;
(S)-1-((S)-2-(3-chlorobenzyl)piperidin-1-yl)-3-(4-(methylsulfonyl)phenoxy)propan-2-ol;
(S)-1-((R)-2-(3-chlorobenzyl)piperidin-1-yl)-3-(4-(methylsulfonyl)phenoxy)propan-2-ol;
N-(4-((S)-3-((trans-3-(3-chlorophenyl)cyclobutyl)amino)-2-hydroxypropoxy)phenyl)-N-methylmethanesulfonamide;
(S)-1-((trans-3-(3-chlorophenyl)cyclobutyl)amino)-3-(4-(methylsulfonyl)phenoxy)propan-2-ol;
(R)-1-((trans-3-(3-chlorophenyl)cyclobutyl)amino)-3-(4-(methylsulfonyl)phenoxy)propan-2-ol;
N-(4-((2S)-3-(3-(3-chlorophenyl)-2,2-dimethylpyrrolidin-1-yl)-2-hydroxypropoxy)phenyl)-N-methylmethanesulfonamide;
N-(4-((S)-3-((S)-3-(3-chlorophenyl)-2,2-dimethylpyrrolidin-1-yl)-2-hydroxypropoxy)phenyl)-N-methylmethanesulfonamide;
N-(4-((S)-3-((R)-3-(3-chlorophenyl)-2,2-dimethylpyrrolidin-1-yl)-2-hydroxypropoxy)phenyl)-N-methylmethanesulfonamide;
N-(4-((2R)-3-(3-(3-chlorophenyl)-2,2-dimethylpyrrolidin-1-yl)-2-hydroxypropoxy)phenyl)-N-methylmethanesulfonamide;
N-(4-((R)-3-((S)-3-(3-chlorophenyl)-2,2-dimethylpyrrolidin-1-yl)-2-hydroxypropoxy)phenyl)-N-methylmethanesulfonamide;
N-(4-((R)-3-((R)-3-(3-chlorophenyl)-2,2-dimethylpyrrolidin-1-yl)-2-hydroxypropoxy)phenyl)-N-methylmethanesulfonamide;
N-(4-((2S)-3-(3-(3-chlorophenyl)pyrrolidin-1-yl)-2-hydroxypropoxy)phenyl)-N-methylmethanesulfonamide;
N-(4-((S)-3-((S)-3-(3-chlorophenyl)pyrrolidin-1-yl)-2-hydroxypropoxy)phenyl)-N-methylmethanesulfonamide;
N-(4-((S)-3-((R)-3-(3-chlorophenyl)pyrrolidin-1-yl)-2-hydroxypropoxy)phenyl)-N-methylmethanesulfonamide;
N-(4-((2R)-3-(3-(3-chlorophenyl)pyrrolidin-1-yl)-2-hydroxypropoxy)phenyl)-N-methylmethanesulfonamide;
N-(4-((R)-3-((S)-3-(3-chlorophenyl)pyrrolidin-1-yl)-2-hydroxypropoxy)phenyl)-N-methylmethanesulfonamide;
N-(4-((R)-3-((R)-3-(3-chlorophenyl)pyrrolidin-1-yl)-2-hydroxypropoxy)phenyl)-N-methylmethanesulfonamide;
N-(4-((2S)-3-(2-(3-chlorophenyl)morpholino)-2-hydroxypropoxy)phenyl)-N-methylmethanesulfonamide;
N-(4-((S)-3-((S)-2-(3-chlorophenyl)morpholino)-2-hydroxypropoxy)phenyl)-N-methylmethanesulfonamide;
N-(4-((S)-3-((R)-2-(3-chlorophenyl)morpholino)-2-hydroxypropoxy)phenyl)-N-methylmethanesulfonamide;
N-(4-((2R)-3-(2-(3-chlorophenyl)morpholino)-2-hydroxypropoxy)phenyl)-N-methylmethanesulfonamide;
N-(4-((R)-3-((R)-2-(3-chlorophenyl)morpholino)-2-hydroxypropoxy)phenyl)-N-methylmethanesulfonamide;
N-(4-((R)-3-((S)-2-(3-chlorophenyl)morpholino)-2-hydroxypropoxy)phenyl)-N-methylmethanesulfonamide;
(S)—N-(4-(3-((3-chlorophenethyl)amino)-2-hydroxypropoxy)phenyl)-N-methylmethanesulfonamide;
(R)—N-(4-(3-((3-chlorophenethyl)amino)-2-hydroxypropoxy)phenyl)-N-methylmethanesulfonamide;
N-(4-((R)-3-(((S)-1-(3-chlorophenyl)propan-2-yl)amino)-2-hydroxypropoxy)phenyl)-N-methylmethanesulfonamide;
N-(4-((R)-3-(((R)-1-(3-chlorophenyl)propan-2-yl)amino)-2-hydroxypropoxy)phenyl)-N-methylmethanesulfonamide;
(S)—N-(4-(3-((2,5-dichlorophenethyl)amino)-2-hydroxypropoxy)phenyl)-N-methylmethanesulfonamide;
(S)—N-(4-(3-((3,5-dichlorophenethyl)amino)-2-hydroxypropoxy)phenyl)-N-methylmethanesulfonamide;
N-(4-((2R)-3-((3-(3-chlorobenzyl)tetrahydrofuran-3-yl)amino)-2-hydroxypropoxy)phenyl)-N-methylmethanesulfonamide;
(S)-1-((3-chlorophenethyl)amino)-3-(4-(methylsulfonyl)phenoxy)propan-2-ol;
(R)-1-((3-chlorophenethyl)amino)-3-(4-(methylsulfonyl)phenoxy)propan-2-ol;
N-(4-((S)-3-(((S)-1-(3-chlorophenyl)propan-2-yl)amino)-2-hydroxypropoxy)phenyl)-N-methylmethanesulfonamide;
N-(4-((S)-3-(((R)-1-(3-chlorophenyl)propan-2-yl)amino)-2-hydroxypropoxy)phenyl)-N-methylmethanesulfonamide;
(R)-1-((1-(3-chlorophenyl)-2-methylpropan-2-yl)amino)-3-(4-(methylsulfonyl)phenoxy)propan-2-ol;
(S)—N-(4-(2-hydroxy-3-((2-methoxyphenethyl)amino)propoxy)phenyl)-N-methylmethanesulfonamide;
(S)—N-(4-(3-((3-cyanophenethyl)amino)-2-hydroxypropoxy)phenyl)-N-methylmethanesulfonamide;
(S)—N-(4-(3-((2-ethylphenethyl)amino)-2-hydroxypropoxy)phenyl)-N-methylmethanesulfonamide;
(S)—N-(4-(3-((2-(difluoromethoxy)phenethyl)amino)-2-hydroxypropoxy)phenyl)-N-methylmethanesulfonamide;
(2R)-1-((2-(3-chlorophenyl)-1-cyclopropylethyl)amino)-3-(4-(methylsulfonyl)phenoxy)propan-2-ol;
(R)—N-(4-(3-((3-chlorophenethyl)amino)-2-hydroxy-2-methylpropoxy)phenyl)-N-methylmethanesulfonamide;
(S)—N-(4-(3-((3-chlorophenethyl)amino)-2-hydroxy-2-methylpropoxy)phenyl)-N-methylmethanesulfonamide;
N-(4-((3-((3-chlorophenethyl)amino)-2-hydroxypropyl)amino)phenyl)-N-methylmethanesulfonamide;
(R)—N-(4-((3-((3-chlorophenethyl)amino)-2-hydroxypropyl)amino)phenyl)-N-methylmethanesulfonamide;
(3S,4S)-1-[2-(3-chlorophenyl)ethyl]-3-{[4-(2-methanesulfonylethanesulfonyl)phenoxy]methyl}-4-methylpyrrolidine;
3-chloro-5-{2-[(3S,4S)-3-{[4-(2-methanesulfonylethanesulfonyl)phenoxy]methyl}-4-methylpyrrolidin-1-yl]ethyl}benzonitrile;
rac-trans-1-[2-(3-chlorophenyl)ethyl]-3-{[4-(2-methoxyethanesulfonyl)phenoxy]methyl}-4-methylpyrrolidine;
(3R,4R)-1-[2-(3-chlorophenyl)ethyl]-3-{[4-(2-methoxyethanesulfonyl)phenoxy]methyl}-4-methylpyrrolidine;
(3S,4S)-1-[2-(3-chlorophenyl)ethyl]-3-{[4-(2-methoxyethanesulfonyl)phenoxy]methyl}-4-methylpyrrolidine;
2-(4-{[(3R,4R)-1-[2-(3-chlorophenyl)ethyl]-4-methylpyrrolidin-3-yl]methoxy}benzenesulfonyl)ethan-1-ol;
2-(4-{[(3S,4S)-1-[2-(3-chlorophenyl)ethyl]-4-methylpyrrolidin-3-yl]methoxy}benzenesulfonyl)ethan-1-ol;
3-chloro-5-[(1S)-1-hydroxy-2-[(2R,4S)-4-[(4-methanesulfonylphenoxy)methyl]-2-methylpyrrolidin-1-yl]ethyl]benzonitrile;
3-chloro-5-[(1R)-1-hydroxy-2-[(2R,4S)-4-[(4-methanesulfonylphenoxy)methyl]-2-methylpyrrolidin-1-yl]ethyl]benzonitrile;
3-[(1R)-1-hydroxy-2-[(2R,4S)-4-[(4-methanesulfonylphenoxy)methyl]-2-methylpyrrolidin-1-yl]ethyl]benzonitrile;
3-[(1S)-1-hydroxy-2-[(2R,4S)-4-[(4-methanesulfonylphenoxy)methyl]-2-methylpyrrolidin-1-yl]ethyl]benzonitrile;
5-[(1R)-1-hydroxy-2-[(2R,4S)-4-[(4-methanesulfonylphenoxy)methyl]-2-methylpyrrolidin-1-yl]ethyl]benzene-1,3-dicarbonitrile;
5-[(1S)-1-hydroxy-2-[(2R,4S)-4-[(4-methanesulfonylphenoxy)methyl]-2-methylpyrrolidin-1-yl]ethyl]benzene-1,3-dicarbonitrile;
3-chloro-5-{1-hydroxy-2-[(3 S,4S)-3-[(4-methanesulfonylphenoxy)methyl]-4-methylpyrrolidin-1-yl]ethyl}benzonitrile;
3-chloro-5-[(1S)-1-hydroxy-2-[(3S,4S)-3-[(4-methanesulfonylphenoxy)methyl]-4-methylpyrrolidin-1-yl]ethyl]benzonitrile;
3-chloro-5-[(1R)-1-hydroxy-2-[(3S,4S)-3-[(4-methanesulfonylphenoxy)methyl]-4-methylpyrrolidin-1-yl]ethyl]benzonitrile;
1-(3-chlorophenyl)-2-((3S,4S)-3-methyl-4-((4-(methylsulfonyl)phenoxy)methyl)pyrrolidin-1-yl)ethan-1-ol;
(1R)-1-(3-chlorophenyl)-2-[(3S,4S)-3-[(4-methanesulfonylphenoxy)methyl]-4-methylpyrrolidin-1-yl]ethan-1-ol;
(1S)-1-(3-chlorophenyl)-2-[(3S,4S)-3-[(4-methanesulfonylphenoxy)methyl]-4-methylpyrrolidin-1-yl]ethan-1-ol;
3-[1-hydroxy-2-[(3S,4S)-3-[(4-methanesulfonylphenoxy)methyl]-4-methylpyrrolidin-1-yl]ethyl]benzonitrile;
3-[(1S)-1-hydroxy-2-[(3S,4S)-3-[(4-methanesulfonylphenoxy)methyl]-4-methylpyrrolidin-1-yl]ethyl]benzonitrile;
3-[(1R)-1-hydroxy-2-[(3S,4S)-3-[(4-methanesulfonylphenoxy)methyl]-4-methylpyrrolidin-1-yl]ethyl]benzonitrile;
5-{1-hydroxy-2-[(3S,4S)-3-[(4-methanesulfonylphenoxy)methyl]-4-methylpyrrolidin-1-yl]ethyl}benzene-1,3-dicarbonitrile;
5-[(1R)-1-hydroxy-2-[(3S,4S)-3-[(4-methanesulfonylphenoxy)methyl]-4-methylpyrrolidin-1-yl]ethyl]benzene-1,3-dicarbonitrile;
5-[(1S)-1-hydroxy-2-[(3S,4S)-3-[(4-methanesulfonylphenoxy)methyl]-4-methylpyrrolidin-1-yl]ethyl]benzene-1,3-dicarbonitrile;
5-{2-[(2R,4S)-4-[(4-methanesulfonylphenoxy)methyl]-2-methylpyrrolidin-1-yl]ethyl}benzene-1,3-dicarbonitrile;
(3S,4S)-1-[2-(3-chlorophenyl)ethyl]-3-[(4-methanesulfonylphenoxy)methyl]-4-methylpyrrolidine;
(2S,5S)-1-[2-(3-chlorophenyl)ethyl]-5-[(4-methanesulfonylphenoxy)methyl]-2-methylpiperidine;
rac-cis-3-{2-[4-[(4-methanesulfonylphenoxy)methyl]-2-methylpyrrolidin-1-yl]ethyl}benzonitrile;
rac-trans-3-{2-[4-[(4-methanesulfonylphenoxy)methyl]-2-methylpyrrolidin-1-yl]ethyl}benzonitrile;
3-{2-[(2S,4R)-4-[(4-methanesulfonylphenoxy)methyl]-2-methylpyrrolidin-1-yl]ethyl}benzonitrile;
3-{2-[(2R,4S)-4-[(4-methanesulfonylphenoxy)methyl]-2-methylpyrrolidin-1-yl]ethyl}benzonitrile;
3-{2-[(2S,4S)-4-[(4-methanesulfonylphenoxy)methyl]-2-methylpyrrolidin-1-yl]ethyl}benzonitrile;
3-{2-[(2R,4R)-4-[(4-methanesulfonylphenoxy)methyl]-2-methylpyrrolidin-1-yl]ethyl}benzonitrile;
5-chloro-3-{2-[(2R,4S)-4-[(4-methanesulfonylphenoxy)methyl]-2-methylpyrrolidin-1-yl]ethyl}-2-methylbenzonitrile;
3-chloro-5-[(2R)-1-[(2R,4S)-4-[(4-methanesulfonylphenoxy)methyl]-2-methylpyrrolidin-1-yl]propan-2-yl]benzonitrile;
3-chloro-5-[(2S)-1-[(2R,4S)-4-[(4-methanesulfonylphenoxy)methyl]-2-methylpyrrolidin-1-yl]propan-2-yl]benzonitrile;
5-chloro-3-{2-[(3S,4S)-3-[(4-methanesulfonylphenoxy)methyl]-4-methylpyrrolidin-1-yl]ethyl}-2-methylbenzonitrile;
5-{2-[(3S,4S)-3-[(4-methanesulfonylphenoxy)methyl]-4-methylpyrrolidin-1-yl]ethyl}benzene-1,3-dicarbonitrile;
3-chloro-5-[1-[(3S,4S)-3-[(4-methanesulfonylphenoxy)methyl]-4-methylpyrrolidin-1-yl]propan-2-yl]benzonitrile;
3-chloro-5-[(2R)-1-[(3S,4S)-3-[(4-methanesulfonylphenoxy)methyl]-4-methylpyrrolidin-1-yl]propan-2-yl]benzonitrile;
3-chloro-5-[(2S)-1-[(3S,4S)-3-[(4-methanesulfonylphenoxy)methyl]-4-methylpyrrolidin-1-yl]propan-2-yl]benzonitrile;
3-{2-[(3S,4S)-3-[(4-methanesulfonylphenoxy)methyl]-4-methylpyrrolidin-1-yl]ethyl}benzonitrile;
rac-cis-1-[2-(3-chlorophenyl)ethyl]-3-[(4-methanesulfonylphenoxy)methyl]-4-methylpyrrolidine;
(3R,4S)-1-[2-(3-chlorophenyl)ethyl]-3-[(4-methanesulfonylphenoxy)methyl]-4-methylpyrrolidine;
(3S,4R)-1-[2-(3-chlorophenyl)ethyl]-3-[(4-methanesulfonylphenoxy)methyl]-4-methylpyrrolidine;
(2R,4S)-1-[2-(3-chlorophenyl)ethyl]-4-[(4-methanesulfonylphenoxy)methyl]-2-methylpyrrolidine;
(3R,4R)-1-[2-(3-chlorophenyl)ethyl]-3-[(4-methanesulfonylphenoxy)methyl]-4-methylpyrrolidine;
(3S,4S)-1-[2-(3-chlorophenyl)ethyl]-3-[(4-methanesulfonylphenoxy)methyl]-4-methylpyrrolidine;
3-{2-[(2R,4S)-4-{[4-(azetidine-3-sulfonyl)phenoxy]methyl}-2-methylpyrrolidin-1-yl]ethyl}benzonitrile;
3-{2-[(2R,4S)-2-methyl-4-({4-[methyl(methylimino)oxo-λ⁶-sulfanyl]phenoxy}methyl)pyrrolidin-1-yl]ethyl}benzonitrile;
3-{2-[(2R,4S)-2-methyl-4-({4-[(R)methyl(methylimino)oxo-λ⁶-sulfanyl]phenoxy}methyl)pyrrolidin-1-yl]ethyl}benzonitrile;
3-{2-[(2R,4S)-2-methyl-4-({4-[(S)methyl(methylimino)oxo-λ⁶-sulfanyl]phenoxy}methyl)pyrrolidin-1-yl]ethyl}benzonitrile;
(2R,5S)-1-[2-(3-chlorophenyl)ethyl]-5-[(4-methanesulfonylphenoxy)methyl]-2-methylpiperidine;
3-{2-[(2R,4S)-4-{[4-(azetidine-3-sulfonyl)phenoxy]methyl}-2-methylpyrrolidin-1-yl]ethyl}-5-chlorobenzonitrile;
3-{2-[(2R,4S)-4-{[4-(3-methanesulfonylpropanesulfonyl)phenoxy]methyl}-2-methylpyrrolidin-1-yl]ethyl}benzonitrile;
(3S,4S)-3-[(4-methanesulfonylphenoxy)methyl]-4-methyl-1-{2-[3-(pentafluoro-λ⁶-sulfanyl)phenyl]ethyl}pyrrolidine;
3-chloro-5-{2-[(2R,4S)-4-{[4-(3-methanesulfonylpropanesulfonyl)phenoxy]methyl}-2-methylpyrrolidin-1-yl]ethyl}benzonitrile;
3-chloro-5-{2-[(2R,4S)-4-{[4-(2-hydroxyethanesulfonyl)phenoxy]methyl}-2-methylpyrrolidin-1-yl]ethyl}benzonitrile;
3-chloro-5-{2-[(3S,4S)-3-{[4-(1-hydroxy-2-methylpropane-2-sulfonyl)phenoxy]methyl}-4-methylpyrrolidin-1-yl]ethyl}benzonitrile;
3-chloro-5-{2-[(3S,4S)-3-{[4-(3-methanesulfonylpropanesulfonyl)phenoxy]methyl}-4-methylpyrrolidin-1-yl]ethyl}benzonitrile;
3-{2-[(3S,4S)-3-{[4-(3-methanesulfonylpropanesulfonyl)phenoxy]methyl}-4-methylpyrrolidin-1-yl]ethyl}benzonitrile;
3-{2-[(3S,4S)-3-methyl-4-({4-[methyl(methylimino)oxo-λ⁶-sulfanyl]phenoxy}methyl)pyrrolidin-1-yl]ethyl}benzonitrile;
3-{2-[(3S,4S)-3-methyl-4-({4-[(R)-methyl(methylimino)oxo-λ⁶-sulfanyl]phenoxy}methyl)pyrrolidin-1-yl]ethyl}benzonitrile;
3-{2-[(3S,4S)-3-methyl-4-({4-[(S)-methyl(methylimino)oxo-λ⁶-sulfanyl]phenoxy}methyl)pyrrolidin-1-yl]ethyl}benzonitrile;
3-{2-[(3S,4S)-3-{[4-(azetidine-3-sulfonyl)phenoxy]methyl}-4-methylpyrrolidin-1-yl]ethyl}benzonitrile;
3-{2-[(3S,4S)-3-{[4-(azetidine-3-sulfonyl)phenoxy]methyl}-4-methylpyrrolidin-1-yl]ethyl}-5-chlorobenzonitrile;
3-chloro-5-{2-[(3S,4S)-3-{[4-(2-hydroxyethanesulfonyl)phenoxy]methyl}-4-methylpyrrolidin-1-yl]ethyl}benzonitrile;
rac-trans-1-[2-(3-chlorophenyl)ethyl]-3-{[4-(3-methanesulfonylpropanesulfonyl)phenoxy]methyl}-4-methylpyrrolidine;
(3R,4R)-1-[2-(3-chlorophenyl)ethyl]-3-{[4-(3-methanesulfonylpropanesulfonyl)phenoxy]methyl}-4-methylpyrrolidine;
(3S,4S)-1-[2-(3-chlorophenyl)ethyl]-3-{[4-(3-methanesulfonylpropanesulfonyl)phenoxy]methyl}-4-methylpyrrolidine;
(3S,4S)-1-[2-(3-chloro-5-fluorophenyl)ethyl]-3-[(4-methanesulfonylphenoxy)methyl]-4-methylpyrrolidine;
3-chloro-5-{2-[(3S)-3-{[4-(2-hydroxyethanesulfonyl)phenoxy]methyl}piperazin-1-yl]ethyl}benzonitrile;
3-chloro-5-{2-[(3S)-3-{[4-(1-hydroxy-2-methylpropane-2-sulfonyl)phenoxy]methyl}piperazin-1-yl]ethyl}benzonitrile;
(3S)-1-[2-(3-chlorophenyl)ethyl]-3-{[4-(3-methanesulfonylpropanesulfonyl)phenoxy]methyl}piperazine;
(3S)-3-{[4-(azetidine-3-sulfonyl)phenoxy]methyl}-1-[2-(3-chlorophenyl)ethyl]piperazine;
3-chloro-5-{2-[(3S)-3-({4-[methyl(methylimino)oxo-λ⁶-sulfanyl]phenoxy}methyl)piperazin-1-yl]ethyl}benzonitrile;
3-chloro-5-{2-[(3S)-3-({4-[(S)-methyl(methylimino)oxo-λ⁶-sulfanyl]phenoxy}methyl)piperazin-1-yl]ethyl}benzonitrile;
3-chloro-5-{2-[(3S)-3-({4-[(R)-methyl(methylimino)oxo-λ⁶-sulfanyl]phenoxy}methyl)piperazin-1-yl]ethyl}benzonitrile;
3-chloro-5-{2-[(3S)-3-[(4-methanesulfonylphenoxy)methyl]piperazin-1-yl]ethyl}benzonitrile;
(3S)-1-[2-(3,5-dichlorophenyl)ethyl]-3-[(4-methanesulfonylphenoxy)methyl]piperazine;
(3S)-1-[2-(5-chloro-2-methoxyphenyl)ethyl]-3-[(4-methanesulfonylphenoxy)methyl]piperazine;
(3S)-1-{2-[5-chloro-2-(difluoromethoxy)phenyl]ethyl}-3-[(4-methanesulfonylphenoxy)methyl]piperazine;
3-{2-[(3S)-3-[(4-methanesulfonylphenoxy)methyl]piperazin-1-yl]ethyl}-5-(trifluoromethyl)benzonitrile;
3-chloro-5-{2-[(3S,4S)-3-[(4-methanesulfonylphenoxy)methyl]-4-methylpyrrolidin-1-yl]ethyl}benzonitrile;
3-fluoro-5-{2-[(3S,4S)-3-[(4-methanesulfonylphenoxy)methyl]-4-methylpyrrolidin-1-yl]ethyl}benzonitrile;
3-bromo-5-{2-[(3S,4S)-3-[(4-methanesulfonylphenoxy)methyl]-4-methylpyrrolidin-1-yl]ethyl}benzonitrile;
(3S)-1-[2-(5-chloro-2-methylphenyl)ethyl]-3-[(4-methanesulfonylphenoxy)methyl]piperazine;
3-{2-[(3S,4S)-3-[(4-methanesulfonylphenoxy)methyl]-4-methylpyrrolidin-1-yl]ethyl}-5-methylbenzonitrile;
rac-cis-3-chloro-5-{2-[4-[(4-methanesulfonylphenoxy)methyl]-2-methylpyrrolidin-1-yl]ethyl}benzonitrile;
3-chloro-5-{2-[(2R,4S)-4-[(4-methanesulfonylphenoxy)methyl]-2-methylpyrrolidin-1-yl]ethyl}benzonitrile;
3-chloro-5-{2-[(2S,4R)-4-[(4-methanesulfonylphenoxy)methyl]-2-methylpyrrolidin-1-yl]ethyl}benzonitrile;
rac-trans-3-chloro-5-{2-[4-[(4-methanesulfonylphenoxy)methyl]-2-methylpyrrolidin-1-yl]ethyl}benzonitrile;
3-chloro-5-(2-((2R,4R)-2-methyl-4-((4-(methylsulfonyl)phenoxy)methyl)pyrrolidin-1-yl)ethyl)benzonitrile;
3-chloro-5-(2-((2S,4S)-2-methyl-4-((4-(methylsulfonyl)phenoxy)methyl)pyrrolidin-1-yl)ethyl)benzonitrile;
rac-cis-1-[2-(3-chlorophenyl)ethyl]-4-[(4-methanesulfonylphenoxy)methyl]-3-methylpyrrolidin-3-ol;
(3R,4R)-1-[2-(3-chlorophenyl)ethyl]-4-[(4-methanesulfonylphenoxy)methyl]-3-methylpyrrolidin-3-ol;
(3S,4S)-1-(3-chlorophenethyl)-3-methyl-4-((4-(methylsulfonyl)phenoxy)methyl)pyrrolidin-3-ol;
rac-trans-1-[2-(3-chlorophenyl)ethyl]-4-[(4-methanesulfonylphenoxy)methyl]-3-methylpyrrolidin-3-ol;
(3R,4S)-1-[2-(3-chlorophenyl)ethyl]-4-[(4-methanesulfonylphenoxy)methyl]-3-methylpyrrolidin-3-ol;
(3S,4R)-1-[2-(3-chlorophenyl)ethyl]-4-[(4-methanesulfonylphenoxy)methyl]-3-methylpyrrolidin-3-ol;
1-[2-(3-chlorophenyl)ethyl]-5-[(4-methanesulfonylphenoxy)methyl]-2-methylpiperazine;
rac-cis-1-[2-(3-chlorophenyl)ethyl]-5-[(4-methanesulfonylphenoxy)methyl]-2-methylpiperazine;
rac-trans-1-[2-(3-chlorophenyl)ethyl]-5-[(4-methanesulfonylphenoxy)methyl]-2-methylpiperazine;
(1R)-1-(3-chlorophenyl)-2-[(2R,4S)-4-[(4-methanesulfonylphenoxy)methyl]-2-methylpyrrolidin-1-yl]ethan-1-ol; and
(1S)-1-(3-chlorophenyl)-2-[(2R,4S)-4-[(4-methanesulfonylphenoxy)methyl]-2-methylpyrrolidin-1-yl]ethan-1-ol,
   or a stereoisomer or tautomer thereof, or a pharmaceutically acceptable salt of any of the foregoing. Isotopically labeled forms of any of the foregoing are also embraced, such as deuterated or tritiated forms (wherein at least one hydrogen is replaced by at least one deuterium or tritium) of any of the specific compounds detailed herein. Mixtures of any of the foregoing are also embraced and described. Prodrugs of any of the foregoing are also embraced herein.

Compound Names included in Table 1 and in the lists in the paragraphs above were generated using ChemDraw® software version 18.1.0.458 or Collaborative Drug Discovery Inc. (CDD) CDD Vault update #3.

### Compositions

Provided herein are pharmaceutical compositions comprising one or more compounds of formula (A), or formula (A′), or any variation or embodiment thereof, as described elsewhere herein, or a stereoisomer or tautomer thereof, or a pharmaceutically acceptable salt of any of the foregoing. In some embodiments, provided herein is a pharmaceutical composition comprising (i) of formula (A), or formula (A′), or any variation or embodiment thereof, as described elsewhere herein, or a stereoisomer or tautomer thereof, or a pharmaceutically acceptable salt of any of the foregoing, and (ii) one or more pharmaceutically acceptable excipients.

Suitable pharmaceutically acceptable excipients may include, for example, fillers, diluents, sterile aqueous solutions and various organic solvents, permeation enhancers, solubilizers, and adjuvants. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Examples of suitable excipients are well-known to those skilled in the art. Such compositions are prepared in a manner well known in the pharmaceutical art. See, e.g., Remington's Pharmaceutical Sciences, Academic Press, 23^{rd} ed. (2020), which is incorporated herein by reference.

The pharmaceutical compositions may be administered in either single or multiple doses. The pharmaceutical composition may be administered by various methods including, for example, oral, rectal, buccal, intranasal, and transdermal routes. In certain embodiments, the pharmaceutical composition may be administered by intra-arterial injection, intravenously, intraperitoneally, parenterally, intramuscularly, subcutaneously, orally, topically, or as an inhalant.

Compounds as described herein may be administered to individuals in a form of generally accepted oral compositions, such as tablets, coated tablets, gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical formulations may contain preservatives, solubilizers, stabilizers, re-wetting agents, emulgators, sweeteners, dyes, adjusters, salts for the adjustment of osmotic pressure, buffers, coating agents or antioxidants.

The specific dose level of a compound as described herein will depend upon a variety of factors such as the age, body weight and sex of the individual as well as the route of administration and other factors. In some embodiments, a dosage is expressed as a number of milligrams of a compound described herein per kilogram of the individual's body weight (mg/kg). Dosages of between about 0.1 mg/kg and 100-150 mg/kg may be appropriate.

The compound may be administered to an individual in accordance with an effective dosing regimen for a desired period of time or duration, such as at least about one month, at least about 2 months, at least about 3 months, at least about 6 months, or at least about 12 months or longer, which in some variations may be for the duration of the individual's life.

### Methods of Treatment

Provided herein is a method of modulating APOL1 in a cell, comprising exposing the cell to an effective amount of a compound of formula (A), or formula (A′), or any variation or embodiment thereof, as described elsewhere herein, or a stereoisomer or tautomer thereof, or a pharmaceutically acceptable salt of any of the foregoing. Also provided herein is a method of modulating APOL1 in a cell, comprising exposing the cell to a composition comprising an effective amount of a compound of formula (A), or formula (A′), or any variation or embodiment thereof, as described elsewhere herein, or a stereoisomer or tautomer thereof, or a pharmaceutically acceptable salt of any of the foregoing, and one or more pharmaceutically acceptable excipients. Isotopically labeled forms of any of the foregoing are also embraced, including, but not limited to, deuterated or tritiated forms (wherein at least one hydrogen is replaced by at least one deuterium or tritium) of any of the specific compounds detailed herein.

Provided herein is a method of inhibiting APOL1 in a cell, comprising exposing the cell to an effective amount of a compound of formula (A), or formula (A′), or any variation or embodiment thereof, as described elsewhere herein, or a stereoisomer or tautomer thereof, or a pharmaceutically acceptable salt of any of the foregoing. Also provided herein is a method of inhibiting APOL1 in a cell, comprising exposing the cell to a pharmaceutical composition comprising an effective amount of a compound of formula (A), or formula (A′), or any variation or embodiment thereof, as described elsewhere herein, or a stereoisomer or tautomer thereof, or a pharmaceutically acceptable salt of any of the foregoing, and one or more pharmaceutically acceptable excipients.

Provided herein is a method of inhibiting APOL1 in an individual, comprising administering to the individual an effective amount of a compound of formula (A), or formula (A′), or any variation or embodiment thereof, as described elsewhere herein, or a stereoisomer or tautomer thereof, or a pharmaceutically acceptable salt of any of the foregoing. Also provided herein is a method of inhibiting APOL1 in an individual, comprising administering to the individual a pharmaceutical composition comprising an effective amount of a compound of formula (A), or formula (A′), or any variation or embodiment thereof, as described elsewhere herein, or a stereoisomer or tautomer thereof, or a pharmaceutically acceptable salt of any of the foregoing, and one or more pharmaceutically acceptable excipients.

In some embodiments, the compounds provided herein inhibit APOL1 at a concentration of less than 10 μM, less than 1 μM, less than 0.5 μM, or less than 0.1 μM. In some embodiments, the compounds provided herein inhibit APOL1 at a concentration of 1 to 10 μM, 0.01 to 1 μM, or 0.01 to 10 μM.

In some embodiments, the compounds provided herein reduce cell death caused by overexpression of APOL1. In some embodiments, the compounds provided herein reduce cell death caused by overexpression APOL1 at a concentration of less than 10 μM, less than 1 μM, less than 0.5 μM, or less than 0.1 μM. In some embodiments, the compounds provided herein reduce cell death caused by APOL1 overexpression at a concentration of 1 to 10 μM, 0.01 to 1 μM, or 0.01 to 10 μM.

In some embodiments, compounds provided herein have an EC₅₀ of less than 1 μM, less than 0.5 μM, or less than 0.1 μM. In some embodiments, the compounds provided herein have an EC₅₀ of 1 to 10 μM, 0.01 to 1 μM, or 0.01 to 10 μM.

In some embodiments, compounds provided herein have an AC₅₀ of less than 1 μM, less than 0.5 μM, or less than 0.1 μM. In some embodiments, the compounds provided herein have an AC₅₀ of 1 to 10 μM, 0.01 to 1 μM, or 0.01 to 10 μM. In some embodiments, the AC₅₀ value reflects the compound's ability to prevent calcium influx by inhibiting APOL1.

In some embodiments, the compounds provided herein inhibit a cation channel. In some embodiments, the compounds of the present disclosure inhibit a calcium channel. In some embodiments, the compounds of the present disclosure reduce calcium transport.

Provided herein is a method of treating an APOL1-mediated disease, disorder, or condition in an individual in need thereof, comprising administering to the individual a therapeutically effective amount of a compound of formula (A), or formula (A′), or any variation or embodiment thereof, as described elsewhere herein, or a stereoisomer or tautomer thereof, or a pharmaceutically acceptable salt of any of the foregoing. Also provided herein is a method of treating an APOL1-mediated disease, disorder, or condition in an individual in need thereof, comprising administering to the individual a pharmaceutical composition comprising a therapeutically effective amount of a compound of formula (A), or formula (A′), or any variation or embodiment thereof, as described elsewhere herein, or a stereoisomer or tautomer thereof, or a pharmaceutically acceptable salt of any of the foregoing, and one or more pharmaceutically acceptable excipients.

Provided herein is a method of treating a kidney disease, disorder, or condition in an individual in need thereof, comprising administering to the individual a therapeutically effective amount of a compound of formula (A), or formula (A′), or any variation or embodiment thereof, as described elsewhere herein, or a stereoisomer or tautomer thereof, or a pharmaceutically acceptable salt of any of the foregoing. Also provided herein is a method of treating a kidney disease, disorder, or condition in an individual in need thereof, comprising administering to the individual a pharmaceutical composition comprising a therapeutically effective amount of a compound of formula (A), or formula (A′), or any variation or embodiment thereof, as described elsewhere herein, or a stereoisomer or tautomer thereof, or a pharmaceutically acceptable salt of any of the foregoing, and one or more pharmaceutically acceptable excipients.

In some embodiments, the individual has a chronic kidney disease. In some embodiments, the individual has hypertension-attributed kidney disease. In some embodiments, the kidney disease, disorder, or condition is an APOL1-mediated kidney disease, disorder, or condition. In some embodiments, the kidney disease, disorder, or condition is selected from the group consisting of focal segmental glomerulosclerosis (FSGS), hypertension-attributed kidney disease, viral nephropathy, COVID-19 associated nephropathy, human immunodeficiency virus-associated nephropathy (HIVAN), sickle-cell nephropathy, lupus nephritis, and diabetic kidney disease.

Also provided herein is a method of treating an APOL1-mediated disorder, such as preeclampsia and sepsis, comprising administering to an individual in need thereof a therapeutically effective amount of a compound of formula (A), or formula (A′), or any variation or embodiment thereof, as described elsewhere herein, or a stereoisomer or tautomer thereof, or a pharmaceutically acceptable salt of any of the foregoing. In some embodiments, the individual is genetically predisposed to developing the APOL1-mediated disorder.

Also provided herein is a method of delaying development of progressive renal allograft loss in a kidney transplant recipient comprising administering to the kidney transplant recipient a therapeutically effective amount of a compound of formula (A), or formula (A′), or any variation or embodiment thereof, as described elsewhere herein, or a stereoisomer or tautomer thereof, or a pharmaceutically acceptable salt of any of the foregoing. In some embodiments, the kidney transplant recipient receives a kidney from a high-risk APOL1 genotype donor. In some embodiments, the kidney transplant recipient is administered a therapeutically effective amount of the compound for a period of time before receiving the kidney transplant. In some embodiments, the kidney transplant recipient is administered a therapeutically effective amount of the compound subsequent to receiving the kidney transplant.

Provided herein is a method of treating a kidney disease, disorder, or condition in an individual in need thereof, comprising administering to the individual a therapeutically effective amount of a compound of formula (A), or formula (A′), or any variation or embodiment thereof, as described elsewhere herein, or a stereoisomer or tautomer thereof, or a pharmaceutically acceptable salt of any of the foregoing, wherein the individual has an APOL1 mutation. Also provided herein is a method of treating a kidney disease, disorder, or condition in an individual in need thereof, comprising administering to the individual a pharmaceutical composition comprising a therapeutically effective amount of a compound of formula (A), or formula (A′), or any variation or embodiment thereof, as described elsewhere herein, or a stereoisomer or tautomer thereof, or a pharmaceutically acceptable salt of any of the foregoing, and one or more pharmaceutically acceptable excipients, wherein the individual has an APOL1 mutation.

The compounds provided herein may also be used in a method of delaying the development of an APOL1-mediated disease, disorder, or condition, comprising administering a compound of formula (A), or formula (A′), or any variation or embodiment thereof, as described elsewhere herein, or a stereoisomer or tautomer thereof, or a pharmaceutically acceptable salt of any of the foregoing, to an individual who is at risk of developing an APOL1-mediated disease, disorder, or condition. In some embodiments, the APOL1-mediated disease, disorder, or condition is preeclampsia or sepsis and the individual has two APOL1 risk alleles. In some embodiments, the APOL1-mediated disease, disorder, or condition is a chronic kidney disease and the individual has any binary combination of G1 and G2 APOL1 risk alleles. In some embodiments, the chronic kidney disease is focal segmental glomerulosclerosis (FSGS), hypertension-attributed kidney disease, human immunodeficiency virus-associated nephropathy (HIVAN), hypertension-attributed kidney disease, sickle cell nephropathy, viral nephropathy, COVID-19 associated nephropathy, lupus nephritis, diabetic kidney disease, or APOL1-associated nephropathy. The compounds as provided herein may also be used in a method of delaying the development of progressive renal allograft loss in an individual who has received a kidney transplantation from a high-risk APOL1 genotype donor.

In some embodiments, the individual has a gain-of-function mutation in APOL1. In some embodiments, the individual has an APOL1 risk allele. In some embodiments, the APOL1 risk allele is a missense variant. In some embodiments, the APOL1 risk allele is a G1 variant. In some embodiments, the G1 variant is G1^{G} (p.S342 G) or G1M (p.I384 M). In some embodiments, the APOL1 risk allele is the G2 variant. In some embodiments, the G2 variant is NYK388-389K. In some embodiments, the APOL1 risk variant is a mutation in the serum resistance-associated (SRA) binding domain of the APOL1 protein.

Also provided herein is a method of inhibiting APOL1 in an individual comprising administering to the individual a therapeutically effective amount of a compound of formula (A), or formula (A′), or any variation or embodiment thereof, as described elsewhere herein, or a stereoisomer or tautomer thereof, or a pharmaceutically acceptable salt of any of the foregoing.

Also provided herein is method of preventing kidney failure in an individual comprising administering a therapeutically effective amount of a compound of Formula (A), or formula (A′) or any variation or embodiment thereof, as described elsewhere herein, or a stereoisomer or tautomer thereof, or a pharmaceutically acceptable salt of any of the foregoing to the individual. In some embodiments, the compound prevents tissue necrosis. In some embodiments, the compound prevents apoptosis. In some embodiments, the compound reduces inflammation.

In some embodiments, the compounds provided herein reduce or eliminate one or more symptoms of a kidney disease. In some embodiments, the compounds reduce nausea, vomiting, loss of appetite, fatigue and weakness, sleep problems, urinary frequency issues, muscle twinges and cramps, swelling, itching, chest pain, shortness of breath, and/or high blood pressure.

In some embodiments, the compounds provided herein reduce the rate of kidney damage and/or progression of kidney damage. In some embodiments, the compounds provided herein reduce the rate of kidney failure. In some embodiments, the compounds provided herein reverse kidney damage. In some embodiments, the compounds reduce the need for dialysis. In some embodiments, the compounds provided herein delay the need for dialysis at least one month, at least two months, at least three months, or at least one year.

In some embodiments, the compounds reduce the rate of or delay the need for a kidney transplant. For example, in some embodiments, the compounds provided herein delay the need for a kidney transplant at least one month, at least two months, at least three months, at least six months, or at least one year. In some embodiments, the compounds provided herein eliminate the need for a kidney transplant.

In some embodiments, the individual has stage 1, stage 2, stage 3A, stage 3B, stage 4, or stage 5 chronic kidney disease. In some embodiments, kidney function is evaluated using an estimated glomerular filtration rate (eGFR) kidney function test.

In some embodiments, the administration is oral administration.

### Kits

The present disclosure further provides kits for carrying out the methods of the invention. The kits may comprise a compound or pharmaceutically acceptable salt thereof as described herein and suitable packaging. The kits may comprise one or more containers comprising any compound described herein. In one aspect, a kit includes a compound of the disclosure or a pharmaceutically acceptable salt thereof, and a label and/or instructions for use of the compound in the treatment of a disease or disorder described herein. The kits may comprise a unit dosage form of the compound.

Provided herein are kits, comprising (i) a compound of formula (A), or formula (A′), or any variation or embodiment thereof, as described elsewhere herein, or a stereoisomer or tautomer thereof, or a pharmaceutically acceptable salt of any of the foregoing, and (ii) instructions for use in treating an APOL1-mediated disease, disorder, or condition in an individual in need thereof. Also provided herein are kits, comprising (i) a pharmaceutical composition comprising a compound of formula (A), or formula (A′), or any variation or embodiment thereof, as described elsewhere herein, or a stereoisomer or tautomer thereof, or a pharmaceutically acceptable salt of any of the foregoing, and one or more pharmaceutically acceptable excipients; and (ii) instructions for use in treating an APOL1-mediated disease, disorder, or condition in an individual in need thereof.

Articles of manufacture are also provided, wherein the article of manufacture comprises a compound of formula (A), or formula (A′), or any variation or embodiment thereof, as described elsewhere herein, or a stereoisomer or tautomer thereof, or a pharmaceutically acceptable salt of any of the foregoing, in a suitable container. Also provided herein are articles of manufacture, comprising a pharmaceutical composition comprising a compound of formula (A), or formula (A′), or any variation or embodiment thereof, as described elsewhere herein, or a stereoisomer or tautomer thereof, or a pharmaceutically acceptable salt of any of the foregoing, in a suitable container. The container may be a vial, jar, ampoule, preloaded syringe, or intravenous bag.

### Methods of Assaying APOL1 Activity

Provided herein is a method of assessing APOL1 inhibition in a cell, comprising inducing APOL1 expression in a cell, contacting the cell with an APOL1 inhibitor, and measuring inhibition of calcium transport. In some embodiments, inducing APOL1 expression comprises contacting the cell with doxycycline. In some embodiments, the cell stably expresses a genetically encoded calcium indicator. In some embodiments, the genetically encoded calcium indicator comprises GCaMP6f. In some embodiments, the cell inducibly expresses APOL1 G2. In some embodiments, the cell stably expresses a genetically encoded calcium indicator and inducibly expresses APOL1 G2. In some embodiments, the APOL1 inhibitor is a compound of formula (A′), or any variation or embodiment thereof, as described elsewhere herein, or a stereoisomer or tautomer thereof, or a pharmaceutically acceptable salt of any of the foregoing

Provided herein is a method of assessing rescue of HEK cell death caused by overexpression of APOL1, inducing APOL1 expression in a cell, contacting the cell with an APOL1 inhibitor, exposing the cell to a luminescence reagent, and measuring luminescence. In some embodiments, inducing APOL1 expression comprises contacting the cell with doxycycline. In some embodiments, the cell overexpresses APOL1G2. In some embodiments, the APOL1 inhibitor is a compound of formula (A′), or any variation or embodiment thereof, as described elsewhere herein, or a stereoisomer or tautomer thereof, or a pharmaceutically acceptable salt of any of the foregoing.

### Methods of Preparing

The present disclosure further provides methods for preparing the compounds of present invention. In some aspect, provided herein are methods of preparing a compound of formula (A), or formula (A′), or any embodiment or variation thereof, such as a compound of formula (I), (II), (III), (B-2), (B-5), or (C-1), or a stereoisomer or tautomer thereof, or a pharmaceutically acceptable salt of any of the foregoing.

In some embodiments, a method for preparing a compound of formula (A) or (A′), or a stereoisomer or tautomer thereof, or a pharmaceutically acceptable salt of any of the foregoing, comprises a step of reacting a compound of formula (A-I1):

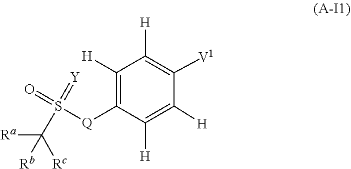

wherein:
Q is absent or is —N—(C₁₋₆alkyl);
Y is O or —N—(C₁₋₆alkyl),
   provided that, when Q is —N(C₁₋₆alkyl), then Y is O;
R^{a}, R^{b}, and R^{c} are each independently H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl, or any two of R^{a}, R^{b}, and R^{c} are taken, together with the atoms to which they are attached, to form a C₃₋₆cycloalkyl or a 3-6 membered heterocyclyl, and the other of R^{a}, R^{b}, and R^{c} is H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl; and
V¹ is selected from the group consisting of:

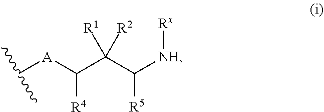

wherein
A is O, NH, N(C₁₋₆alkyl), CH₂, or CH(C₁₋₆alkyl);
R^{x} is H,
or R^{x} is taken together with one of R¹ and R², and the atoms to which they are attached, to form a 3-8 membered heterocyclyl, wherein the 3-8 membered heterocyclyl is substituted with n independently selected R^{g} substituents, wherein n is an integer from 0-6, and R^{g} is —OH, halo, C₁₋₆alkyl, or C₁₋₆alkoxy;
R¹ and R² are independently H, halo, or —OH,
or one of R¹ and R² is taken together with R^{x}, and the atoms to which they are attached, to form a 3-8 membered heterocyclyl, wherein the 3-8 membered heterocyclyl is substituted with n independently selected R^{g} substituents, wherein n is an integer from 0-6, and R^{g} is —OH, halo, C₁₋₆alkyl, or C₁₋₆alkoxy, and the other of R¹ and R² is H, halo, or —OH;
R⁴ and R⁵ are independently H,
or R⁴ and R⁵ are taken, together with the atoms to which they are attached, to form a C₃₋₈cycloalkyl,
provided that either:
   (1) R^{x} is taken together with one of R¹ and R², and the atoms to which they are attached, to form a 3-8 membered heterocyclyl, wherein the 3-8 membered heterocyclyl is substituted with n independently selected R^{g} substituents, wherein n is an integer from 0-6, and R^{g} is —OH, halo, C₁₋₆alkyl, or C₁₋₆alkoxy, or
   (2) R⁴ and R⁵ are taken, together with the atoms to which they are attached, to form a C₃₋₈cycloalkyl,

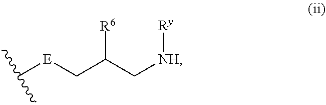

wherein
E is O, NH, N(C₁₋₆alkyl), CH₂, or CH(C₁₋₆alkyl);
R⁶ is H or —OH; and
R^{y} is H; and

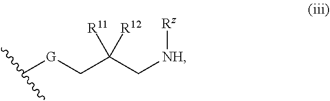

wherein
G is O, NH, N(C₁₋₆alkyl), CH₂, or CH(C₁₋₆alkyl);
R^{z} is H or C₁₋₆alkyl, wherein the C₁₋₆alkyl is optionally substituted with one or more C₃₋₈cycloalkyl; and
R¹¹ and R¹² are independently H, —OH, halo, or C₁₋₆alkyl;
with:
a compound of formula (A-I2):

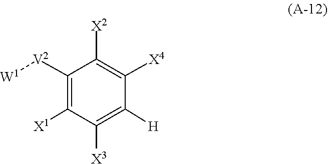

wherein:
the dashed line represents a single or double bond;
W¹ is oxo, halo, or sulfonate ester;
V² is selected from the group consisting of:

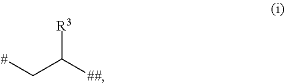

wherein
R³ is H, —OH, halo, or C₁₋₆alkoxy;

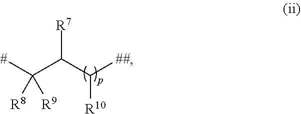

wherein
p is 0 or 1,
   provided that, when p is 1, then E is O;
R⁷ is H;
one of R⁸ and R⁹ is taken together with R¹⁰, and the atoms to which they are attached, to form a C₃₋₈cycloalkyl, and the other of R⁸ and R⁹ is H or C₁₋₆alkyl; and
R¹⁰ is taken together with one of R⁸ and R⁹, and the atoms to which they are attached, to form a C₃₋₈cycloalkyl; and

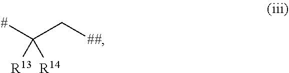

wherein
R¹³ and R¹⁴ are independently H, C₁₋₆alkyl, or C₃₋₈cycloalkyl,
or R¹³ and R¹⁴ are taken, together with the atoms to which they are attached, to form a 3-8 membered heterocyclyl,
wherein, for each of (i)-(iii), #denotes the point of attachment to W¹, and ##denotes the point of attachment to the phenyl ring bearing moieties X¹-X⁴; and
X¹, X², X³, and X⁴ are, independently of each other, H, halo, —CN, C₁₋₆alkyl, C₁₋₆alkoxy, or SF₅, wherein the C₁₋₆alkyl or C₁₋₆alkoxy is optionally substituted with one or more halo,
   provided that at least one of X¹, X², X³, and X⁴ is halo, —CN, C₁₋₆alkyl, C₁₋₆alkoxy, or SF₅, wherein the C₁₋₆alkyl or C₁₋₆alkoxy is optionally substituted with one or more halo;
to give a compound of formula (A) or (A′) or a stereoisomer or tautomer thereof, or a pharmaceutically acceptable salt of any of the foregoing.

In some embodiments, the compound of formula (A) or (A′) is prepared by a step comprising:
a) alkylation of an amine of formula (A-I1) with an alkyl halide, or sulfonate ester compound of formula (A-I2) in the presence of an inorganic or organic base; or
b) reductive amination of an aldehyde or ketone of formula (A-I2) with an amine of formula (A-I1) in the presence of a reducing agent.

In some embodiments, the compound of formula (A) or (A′) is prepared by a step comprising alkylation of an amine of formula (A-I1) with an alkyl halide, or sulfonate ester compound of formula (A-I2) in the presence of an inorganic or organic base. In some embodiments, the inorganic base is selected from the group consisting of potassium carbonate, sodium carbonate, and sodium bicarbonate. In some embodiments, the organic base is a tertiary amine. In some embodiments, the organic base is selected from the group consisting of trimethylamine, triethylamine, and diisopropylethyamine.

In some embodiments, the sulfonate ester compound of formula (A-I2) is a mesylate or a tosylate. In some embodiments, the sulfonate ester compound of formula (A-I2) is a mesylate. In some embodiments, the sulfonate ester compound of formula (A-I2) is a tosylate.

In some embodiments, the compound of formula (A) or (A′) is prepared by a step comprising reductive amination of an aldehyde or ketone of formula (A-I2) with an amine of formula (I-I1). In some embodiments, the reductive amination proceeds under the action of a reducing agent. In some embodiments, the reducing agent is sodium triacetoxyborohydride, or sodium cyanoborohydride.

In some embodiments, a method for preparing a compound of formula (A) or (A′), or a stereoisomer or tautomer thereof, or a pharmaceutically acceptable salt of any of the foregoing, comprises a step of reacting a compound of formula (A-I3):

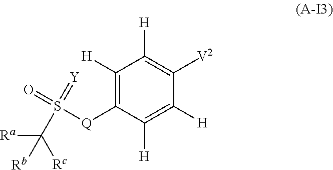

wherein:
Q is absent or is —N—(C₁₋₆alkyl);
Y is O or —N—(C₁₋₆alkyl),
   provided that, when Q is —N(C₁₋₆alkyl), then Y is O;
R^{a}, R^{b}, and R^{c} are each independently H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl, or any two of R^{a}, R^{b}, and R^{c} are taken, together with the atoms to which they are attached, to form a C₃₋₆cycloalkyl or a 3-6 membered heterocyclyl, and the other of R^{a}, R^{b}, and R^{c} is H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl; and
V² is halo or OH,
with:
a compound of formula (A-I4):

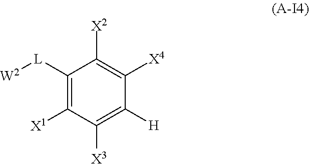

wherein:
W² is H, or sulfamate;
L is selected from the group consisting of:

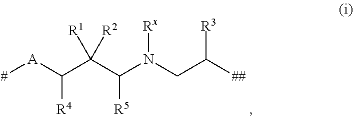

wherein
A is O, NH, N(C₁₋₆alkyl), CH₂, or CH(C₁₋₆alkyl);
R^{x} is H,
or R^{x} is taken together with one of R¹ and R², and the atoms to which they are attached, to form a 3-8 membered heterocyclyl, wherein the 3-8 membered heterocyclyl is substituted with n independently selected R^{g} substituents, wherein n is an integer from 0-6, and R^{g} is —OH, halo, C₁₋₆alkyl, or C₁₋₆alkoxy;
R¹ and R² are independently H, halo, or —OH,
or one of R¹ and R² is taken together with R^{x}, and the atoms to which they are attached, to form a 3-8 membered heterocyclyl, wherein the 3-8 membered heterocyclyl is substituted with n independently selected R^{g} substituents, wherein n is an integer from 0-6, and R^{g} is —OH, halo, C₁₋₆alkyl, or C₁₋₆alkoxy, and the other of R¹ and R² is H, halo, or —OH;
R³ is H, —OH, halo, or C₁₋₆alkoxy; and
R⁴ and R⁵ are independently H,
or R⁴ and R⁵ are taken, together with the atoms to which they are attached, to form a C₃₋₈cycloalkyl,
provided that either:
   (1) R^{x} is taken together with one of R¹ and R², and the atoms to which they are attached, to form a 3-8 membered heterocyclyl, wherein the 3-8 membered heterocyclyl is substituted with n independently selected R^{g} substituents, wherein n is an integer from 0-6, and R^{g} is —OH, halo, C₁₋₆alkyl, or C₁₋₆alkoxy, or
   (2) R⁴ and R⁵ are taken, together with the atoms to which they are attached, to form a C₃₋₈cycloalkyl,

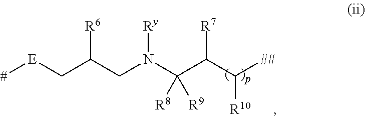

wherein
E is O, NH, N(C₁₋₆alkyl), CH₂, or CH(C₁₋₆alkyl);
p is 0 or 1,
   provided that, when p is 1, then E is O;
R⁶ is H or —OH;
R^{y} is H,
or R^{y} is taken together with R⁷, and the atoms to which they are attached, to form a 3-8 membered heterocyclyl,
or R^{y} is taken together with one of R⁸ and R⁹, and the atoms to which they are attached, to form a 3-8 membered heterocyclyl;
R⁷ is H,
or R⁷ is taken together with R^{y}, and the atoms to which they are attached, to form a 3-8 membered heterocyclyl;
R⁸ and R⁹ are independently H or C₁₋₆alkyl,
or one of R⁸ and R⁹ is taken together with R^{y}, and the atoms to which they are attached, to form a 3-8 membered heterocyclyl, and the other of R⁸ and R⁹ is H or C₁₋₆alkyl, or one of R⁸ and R⁹ is taken together with R¹⁰, and the atoms to which they are attached, to form a C₃₋₈cycloalkyl, and the other of R⁸ and R⁹ is H or C₁₋₆alkyl; and
R¹⁰ is H,
or R¹⁰ is taken together with one of R⁸ and R⁹, and the atoms to which they are attached, to form a C₃₋₈cycloalkyl,
provided that:
   (1) R^{y} is taken together with R⁷, and the atoms to which they are attached, to form a 3-8 membered heterocyclyl, or
   (2) R^{y} is taken together with one of R⁸ and R⁹, and the atoms to which they are attached, to form a 3-8 membered heterocyclyl, or
   (3) one of R⁸ and R⁹ is taken together with R¹⁰ and the atoms to which they are attached, to form a C₃₋₈cycloalkyl, and

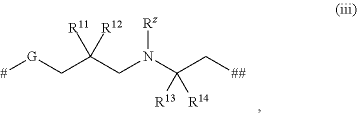

wherein
G is O, NH, N(C₁₋₆alkyl), CH₂, or CH(C₁₋₆alkyl);
R^{z} is H or C₁₋₆alkyl, wherein the C₁₋₆alkyl is optionally substituted with one or more C₃₋₈cycloalkyl;
R¹¹ and R¹² are independently H, —OH, halo, or C₁₋₆alkyl; and
R¹³ and R¹⁴ are independently H, C₁₋₆alkyl, or C₃₋₈cycloalkyl,
or R¹³ and R¹⁴ are taken, together with the atoms to which they are attached, to form a 3-8 membered heterocyclyl,
wherein, for each of (i)-(iii), #denotes the point of attachment to the phenyl ring bearing moiety Q, and ##denotes the point of attachment to the phenyl ring bearing moieties X¹-X⁴; and
X¹, X², X³, and X⁴ are, independently of each other, H, halo, —CN, C₁₋₆alkyl, C₁₋₆alkoxy, or SF₅, wherein the C₁₋₆alkyl or C₁₋₆alkoxy is optionally substituted with one or more halo,
   provided that at least one of X¹, X², X³, and X⁴ is halo, —CN, C₁₋₆alkyl, or C₁₋₆alkoxy, or SF₅, wherein the C₁₋₆alkyl or C₁₋₆alkoxy is optionally substituted with one or more halo;
to give a compound of formula (A) or (A′) or a stereoisomer or tautomer thereof, or a pharmaceutically acceptable salt of any of the foregoing.

In some embodiments, the compound of formula (A) or (A′) is prepared by a step comprising alkylation of an alcohol or amine of formula (A-I4) with an alkyl halide compound of formula (A-I3) in the presence of an inorganic or organic base. In some embodiments, the inorganic base is selected from the group consisting of potassium carbonate, sodium carbonate, and sodium bicarbonate. In some embodiments, the organic base is a tertiary amine. In some embodiments, the organic base is selected from the group consisting of trimethylamine, triethylamine, and diisopropylethyamine.

In some embodiments, the compound of formula (A) or (A′) is prepared by a step comprising alkylation of an alcohol of formula (A-I3) with a sulfamate compound of formula (A-I4) in the presence of an inorganic or organic base. In some embodiments, the inorganic base is selected from the group consisting of potassium carbonate, sodium carbonate, and sodium bicarbonate. In some embodiments, the organic base is a tertiary amine. In some embodiments, the organic base is selected from the group consisting of trimethylamine, triethylamine and diisopropylethyamine.

In some embodiments, a method for preparing a compound of formula (A) or (A′), or a stereoisomer or tautomer thereof, or a pharmaceutically acceptable salt of any of the foregoing, comprises a step of reacting a compound of formula (A-I5):

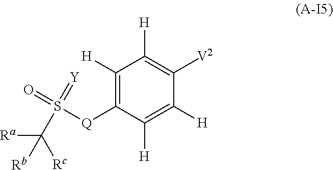

wherein:
Q is absent or is —N—(C₁₋₆alkyl);
Y is O or —N—(C₁₋₆alkyl),
   provided that, when Q is —N(C₁₋₆alkyl), then Y is O;
R^{a}, R^{b}, and R^{c} are each independently H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl, or any two of R^{a}, R^{b}, and R^{c} are taken, together with the atoms to which they are attached, to form a C₃₋₆cycloalkyl or a 3-6 membered heterocyclyl, and the other of R^{a}, R^{b}, and R^{c} is H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl; and
V³ is selected from the group consisting of:

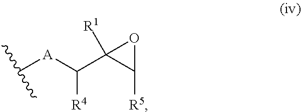

wherein
A is O, NH, N(C₁₋₆alkyl), CH₂, or CH(C₁₋₆alkyl);
R¹ is H, halo, or —OH,
R⁴ and R⁵ are taken, together with the atoms to which they are attached, to form a C₃₋₈cycloalkyl,

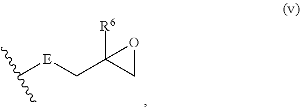

wherein
E is O, NH, N(C₁₋₆alkyl), CH₂, or CH(C₁₋₆alkyl);
R⁶ is H or —OH; and

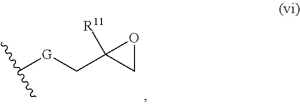

wherein
G is O, NH, N(C₁₋₆alkyl), CH₂, or CH(C₁₋₆alkyl);
R¹¹ is H, —OH, halo, or C₁₋₆alkyl;
with:
a compound of formula (A-I6):

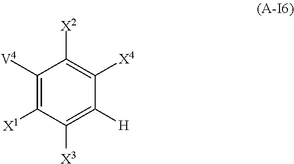

wherein
V⁴ is selected from the group consisting of:

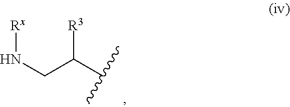

wherein
R^{x} is H, and
R³ is H, —OH, halo, or C₁₋₆alkoxy;

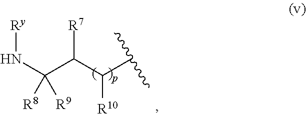

wherein
p is 0 or 1,
   provided that, when p is 1, then E is O;
R^{y} is H,
or R^{y} is taken together with R⁷, and the atoms to which they are attached, to form a 3-8 membered heterocyclyl,
or R^{y} is taken together with one of R⁸ and R⁹, and the atoms to which they are attached, to form a 3-8 membered heterocyclyl;
R⁷ is H,
or R⁷ is taken together with R^{y}, and the atoms to which they are attached, to form a 3-8 membered heterocyclyl;
R⁸ and R⁹ are independently H or C₁₋₆alkyl,
or one of R⁸ and R⁹ is taken together with R^{y}, and the atoms to which they are attached, to form a 3-8 membered heterocyclyl, and the other of R⁸ and R⁹ is H or C₁₋₆alkyl,
or one of R⁸ and R⁹ is taken together with R¹⁰, and the atoms to which they are attached, to form a C₃₋₈cycloalkyl, and the other of R⁸ and R⁹ is H or C₁₋₆alkyl; and
R¹⁰ is H,
or R¹⁰ is taken together with one of R⁸ and R⁹, and the atoms to which they are attached, to form a C₃₋₈cycloalkyl,
provided that:
   (1) R^{y} is taken together with R⁷, and the atoms to which they are attached, to form a 3-8 membered heterocyclyl, or
   (2) R^{y} is taken together with one of R⁸ and R⁹, and the atoms to which they are attached, to form a 3-8 membered heterocyclyl, or
   (3) one of R⁸ and R⁹ is taken together with R¹⁰ and the atoms to which they are attached, to form a C₃₋₈cycloalkyl, and

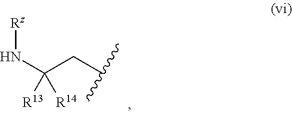

wherein
R¹³ and R¹⁴ are independently H, C₁₋₆alkyl, or C₃₋₈cycloalkyl,
or R¹³ and R¹⁴ are taken, together with the atoms to which they are attached, to form a 3-8 membered heterocyclyl; and
X¹, X², X³, and X⁴ are, independently of each other, H, halo, —CN, C₁₋₆alkyl, C₁₋₆alkoxy, or SF₅, wherein the C₁₋₆alkyl or C₁₋₆alkoxy is optionally substituted with one or more halo,
   provided that at least one of X¹, X², X³, and X⁴ is halo, —CN, C₁₋₆alkyl, C₁₋₆alkoxy, or SF₅, wherein the C₁₋₆alkyl or C₁₋₆alkoxy is optionally substituted with one or more halo;
to give a compound of formula (A) or (A′) or a stereoisomer or tautomer thereof, or a pharmaceutically acceptable salt of any of the foregoing.

In some embodiments, the compound of formula (A) or (A′) is prepared by a step comprising alkylation of an epoxide compound of formula (A-I5) with an amine compound of formula (A-I6) in the presence of an organic base. In some embodiments, the organic base is a tertiary amine. In some embodiments, the organic base is selected from the group consisting of trimethylamine, triethylamine and diisopropylethyamine.

### EXAMPLES

The following synthetic reaction schemes, which are detailed in the Schemes, General Procedures, and Examples, are merely illustrative of some of the methods by which the compounds of the present disclosure, or an embodiment or aspect thereof, can be synthesized. Various modifications to these synthetic reaction schemes can be made, as will be apparent to those of ordinary skill in the art.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Although certain exemplary embodiments are depicted and described herein, the compounds of the present disclosure, or any variation or embodiment thereof, may be prepared using appropriate starting materials according to the methods described generally herein and/or by methods available to one of ordinary skill in the art.

### Synthetic Examples

As depicted in the Schemes, General Procedures, and Examples below, in certain exemplary embodiments, compounds of formula (A), or formula (A′), or any variation or embodiment thereof, as described elsewhere herein, or a stereoisomer or tautomer thereof, or a pharmaceutically acceptable salt of any of the foregoing, are prepared according to the general procedures. The general methods below, and other methods known to synthetic chemists of ordinary skill in the art, can be applied to all formulae, variations, embodiments, and species described herein.

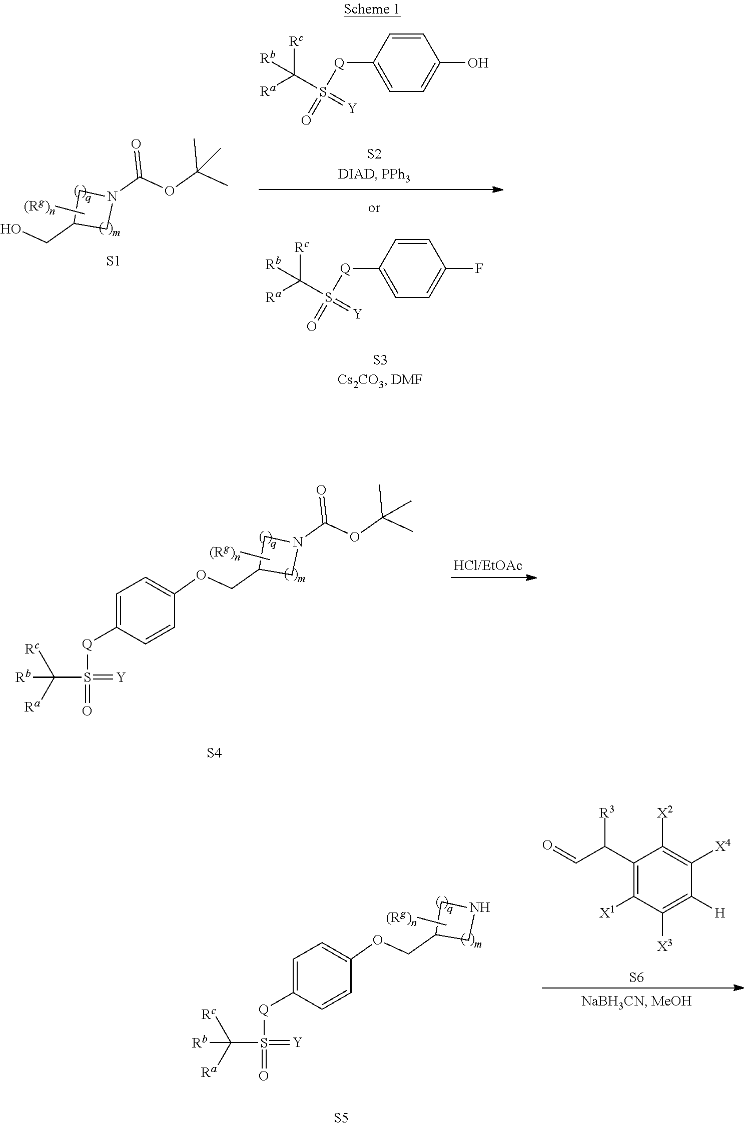

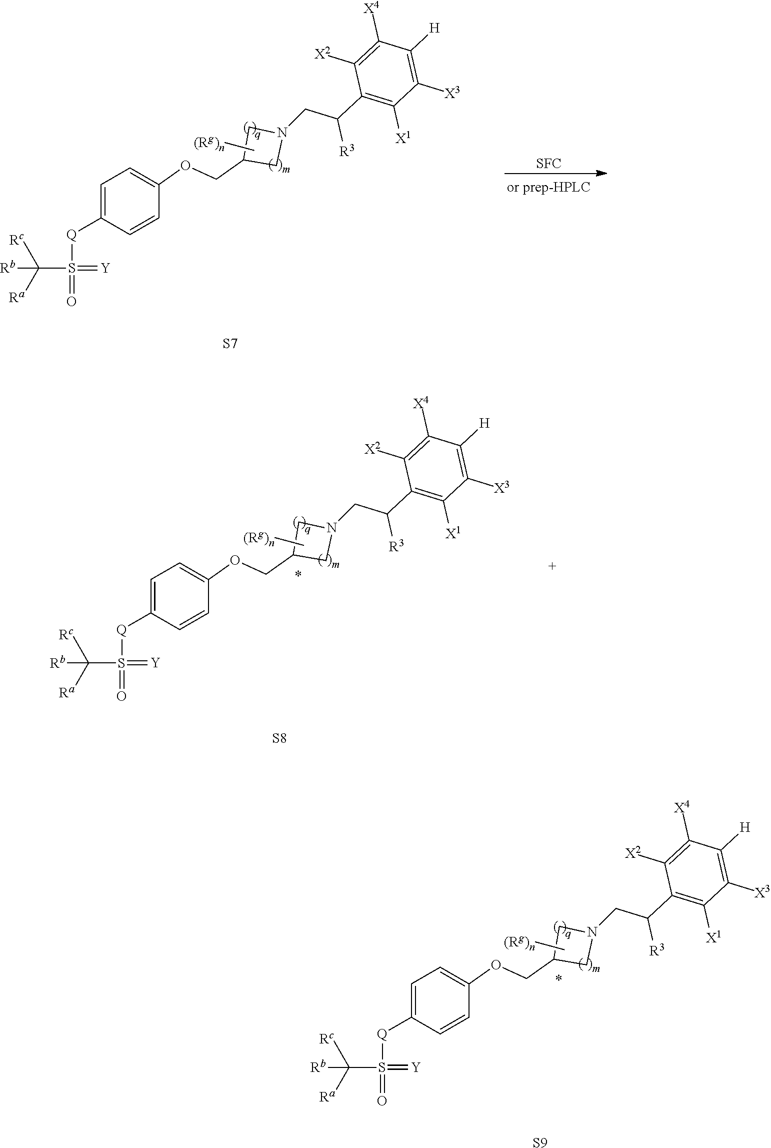

Compounds of formulae S7-S9 may be prepared by the general synthetic method shown in Scheme 1. It is to be understood that, where applicable, the moieties and variables depicted in Scheme 1 are as defined elsewhere herein for a compound of formula (A), or formula (A′), or any variation or embodiment thereof, or a stereoisomer or tautomer thereof, or a pharmaceutically acceptable salt of any of the foregoing. In addition, with reference to Scheme 1, m is an integer from 0-5 and q is an integer from 0-5, provided that 1≤(m+q)≤6.

C—O bond formation may be accomplished through either a Mitsunobu reaction with phenols of formula S2 or an S_{N}Ar with aryl fluorides of formula S3 to provide compounds of formula S4. Deprotection of the N-tert-butyloxycarbonyl (Boc) group may proceed using a protic acid such as hydrochloric acid to give compounds of formula S5. Compounds of formula S7 can be prepared through reductive amination using an aldehyde of formula S6 and a hydride source such as NaBH₃CN. Chiral preparative SFC or HPLC separation may be utilized to provide two or more single stereoisomers of formulas S8 and S9.

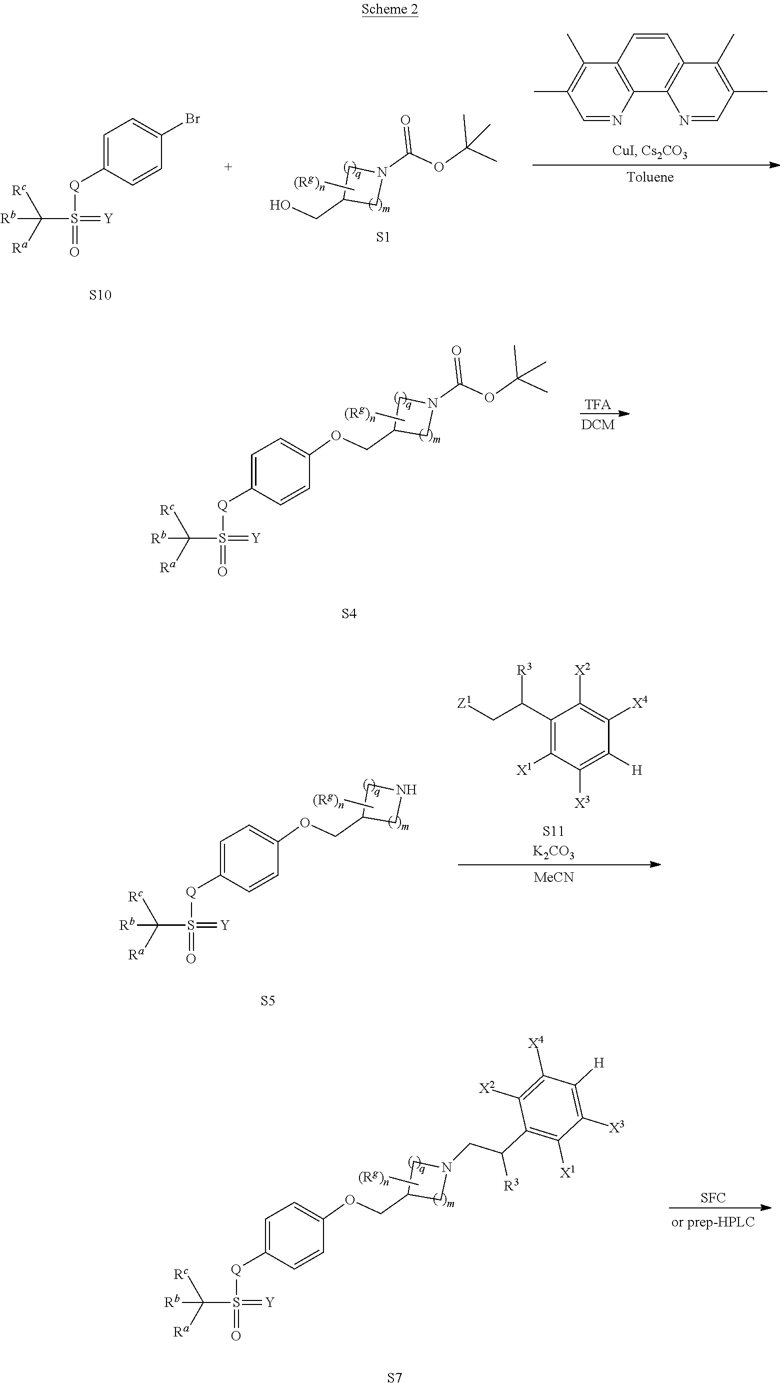

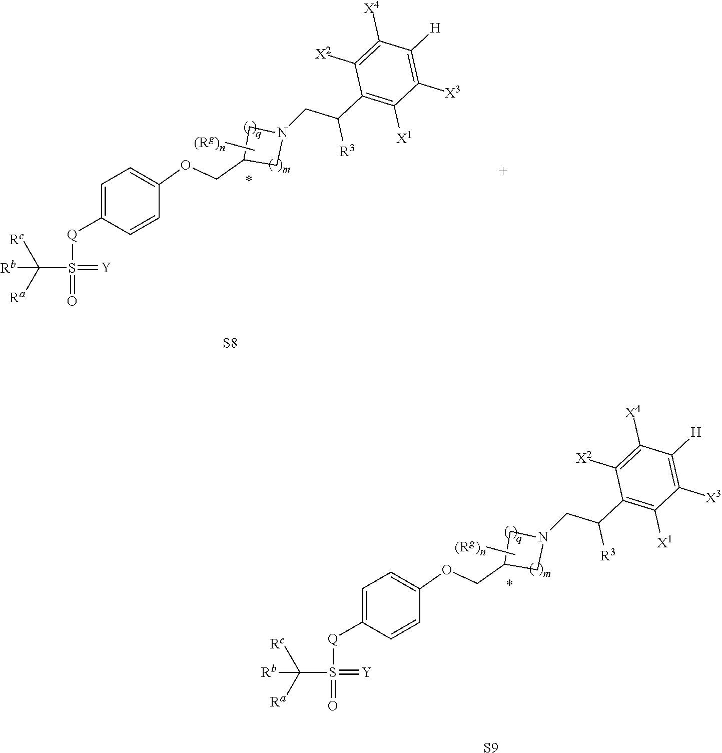

Compounds of formulas S7-S9 may be prepared by the alternative general synthetic method shown in Scheme 2. It is to be understood that, where applicable, the moieties and variables depicted in Scheme 2 are as defined elsewhere herein for a compound of formula (A), or formula (A′), or any variation or embodiment thereof, or a stereoisomer or tautomer thereof, or a pharmaceutically acceptable salt of any of the foregoing. In addition, with reference to Scheme 2: m is an integer from 0-5 and q is an integer from 0-5, provided that 1≤(m+q)≤6; and Z¹ is halo (for example: chloro or bromo) or a sulfonate ester (for example: mesylate).

C—O coupling of an aryl halide of formula S10 with an alcohol of formula S1 may be performed using conditions such as copper(I) iodide, Cs₂CO₃, and 3,4,7,8-tetramethyl-1,10-phenanthroline in toluene at an elevated temperature to afford compounds of formula S4. Deprotection of the N-tert-butyloxycarbonyl (Boc) group may proceed using a protic acid such as trifluoroacetic acid to give compounds of formula S5. Compounds of formula S7 can be prepared using a base such as K₂CO₃, an alkyl halide or alkyl sulfonate ester such as alkyl bromide of formula S11, and an organic solvent such as MeCN at elevated temperature. Chiral preparative SFC or HPLC separation may be utilized to provide two or more single stereoisomers of formulas S8 and S9.

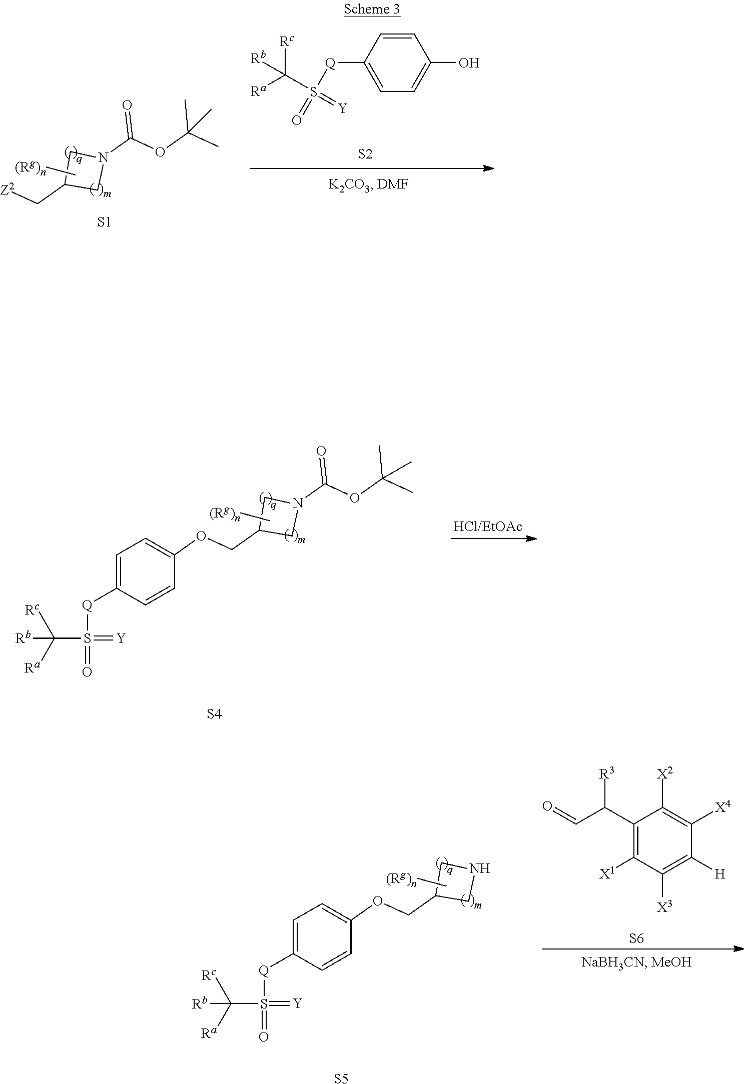

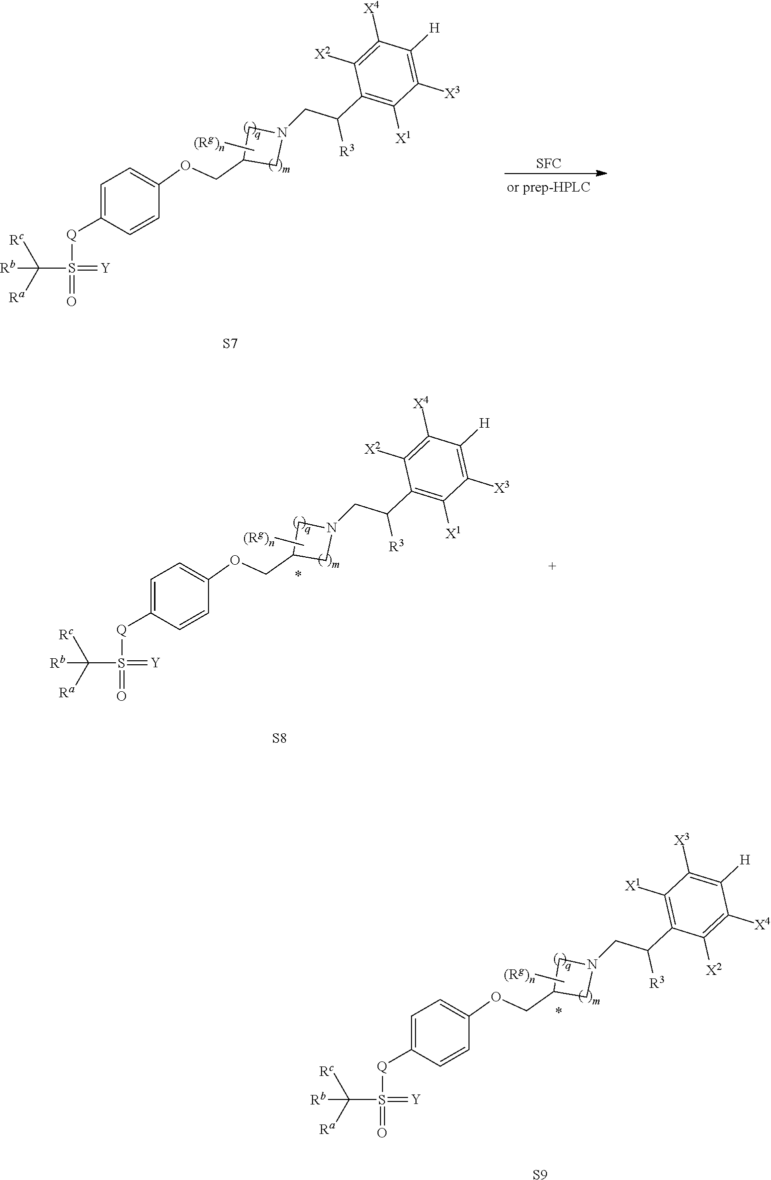

Compounds of formulas S7-S9 may be prepared by the alternative general synthetic method shown in Scheme 3. It is to be understood that, where applicable, the moieties and variables depicted in Scheme 3 are as defined elsewhere herein for a compound of formula (A), or formula (A′), or any variation or embodiment thereof, or a stereoisomer or tautomer thereof, or a pharmaceutically acceptable salt of any of the foregoing. In addition, with reference to Scheme 3: m is an integer from 0-5 and q is an integer from 0-5, provided that 1≤(m+q)≤6; and Z² is halo (for example: chloro or bromo) or a sulfonate ester (for example: mesylate).

C—O bond formation may be accomplished through an S_{N}2 reaction with alkyl halide or alkyl sulfonate ester such as alkyl bromide of formula S12 with phenols of formula S2 in the presence of bases such as K₂CO₃ to give compounds of formula S4. Deprotection of the N-tert-butyloxycarbonyl (Boc) group may proceed using a protic acid such as HCl to give compounds of formula S5. Compounds of formula S7 can be prepared through reductive amination using an aldehyde of formula S6 and a hydride source such as NaBH₃CN. Chiral preparative SFC or HPLC separation may be utilized to provide two or more single stereoisomers of formula S8 and S9.

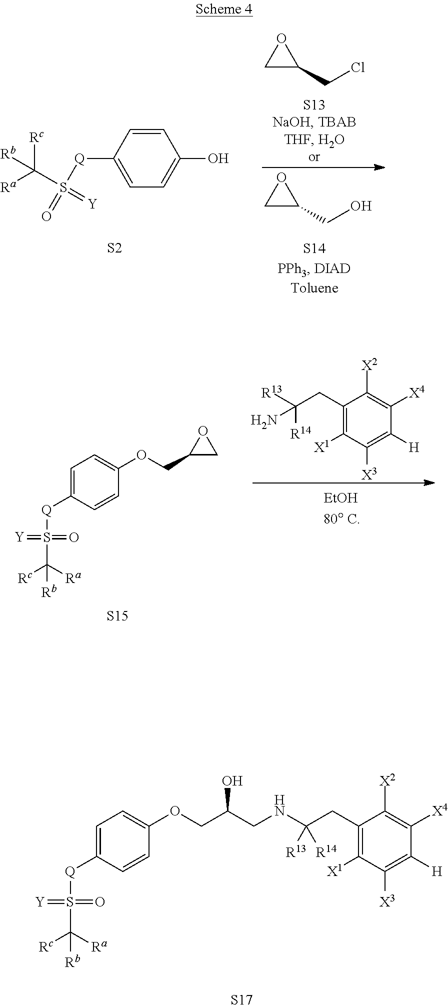

Compounds of formula S17 may be prepared by the alternative general synthetic method shown in Scheme 4. It is to be understood that, where applicable, the moieties and variables depicted in Scheme 4 are as defined elsewhere herein for a compound of formula (A), or formula (A′), or any variation or embodiment thereof, or a stereoisomer or tautomer thereof, or a pharmaceutically acceptable salt of any of the foregoing.

Compounds of formula S17 with (S)-stereochemistry may be accessed by treating phenols of formula S2 with (R)-2-(chloromethyl)oxirane (S13) in the presence of a phase transfer catalyst such as TBAB and a base such as NaOH. Alternately, treatment of phenol S2 with (R)-oxiran-2-ylmethanol S14 and Mitsunobu conditions such as PPh₃ and DIAD may also provide (S)—S15. Epoxide opening to provide compounds of formula S17 proceeds in the presence of amines of formula S16, triethylamine in cases where the salt form of amine S16 is used, as a solution in ethanol at elevated temperatures. In cases where epimeric —OH stereochemistry of compounds of formula S17 is desired, the above synthetic scheme may be modified to utilize (S)-2-(chloromethyl)oxirane or (S)-oxiran-2-ylmethanol.

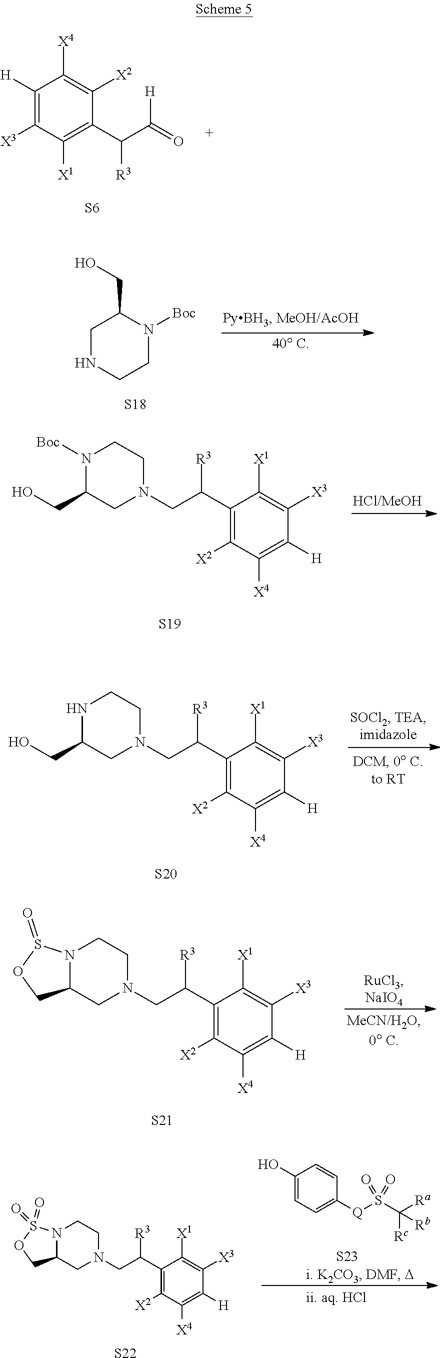

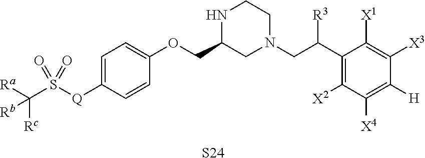

Compounds of formula S24 may be prepared according to Scheme 5. Reductive amination of a mono-protected piperazine such as S18 with an aldehyde such as S6 gives compound S19. Removal of the N-tert-butyloxycarbonyl (Boc) group upon treatment with a protic acid such as HCl in a solvent such as MeOH gives S20. Treatment with thionyl chloride, triethylamine, and imidazole gives rise to S21. Oxidation to the oxathiazolidine-2,2-dioxide occurs on treatment with ruthenium (III) chloride and sodium periodate in a mixed solvent system of acetonitrile and water to give S22. Heating S22 with a phenol such as S23 and potassium carbonate in DMF, followed by treatment with aqueous HCl, gives compounds of formula S24. Chiral preparative SFC or HPLC separation may be utilized to provide two or more single stereoisomers of compounds derived from formula S24.

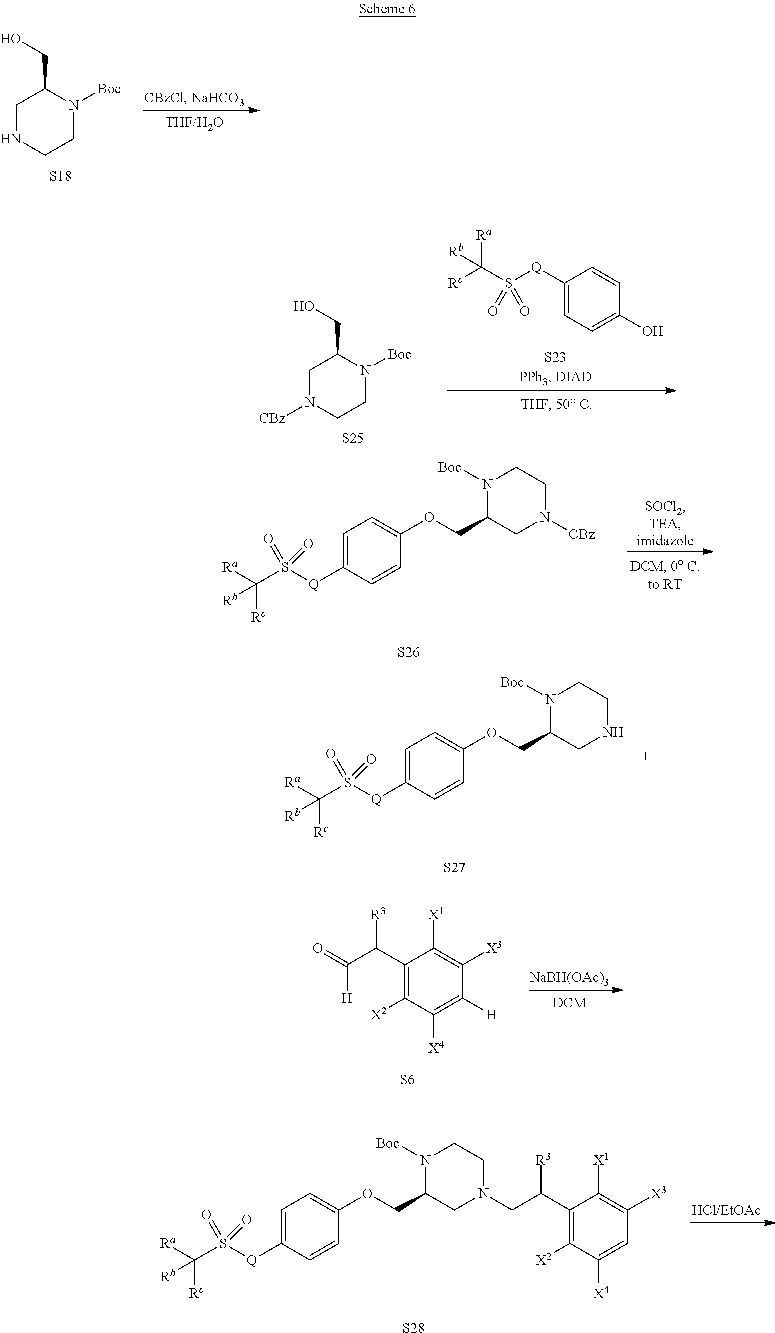

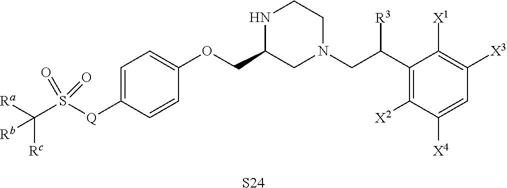

An alternative synthetic approach to compounds of formula S24 is depicted in Scheme 6. Reaction of piperazine S18 with benzyl chloroformate in the presence of a base such as sodium bicarbonate generates the bis-carbamate S25. Mitsunobu coupling of phenol S23 with triphenylphosphine and DIAD in THF generates S26. Selective cleavage of the benzyl carbamate may be achieved by treatment with thionyl chloride, triethylamine, and imidazole in DCM to give S27, which may then undergo reductive amination using sodium triacetoxyborohydride provides compounds of formula S28. Cleavage of the N-tert-butyloxycarbonyl (Boc) group provides S24. Chiral preparative SFC or HPLC separation may be utilized to provide two or more single stereoisomers of compounds derived from formula S24.

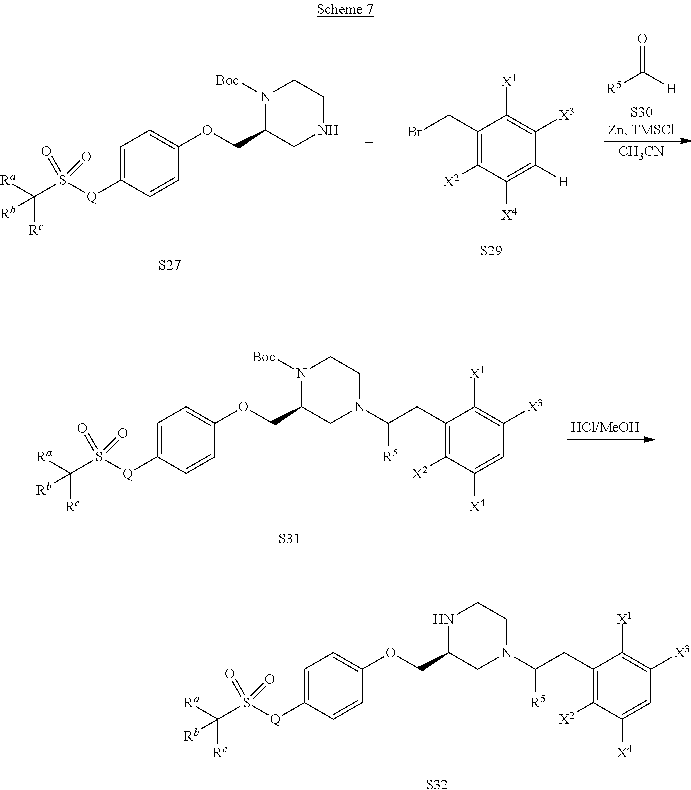

Scheme 7 outlines an approach to compounds of formula S32. Addition of an alkylzinc reagent generated in situ from benzyl bromide S29 to an iminium ion formed by reaction of piperazine S27 and aldehyde S30 gives S31. Removal of the N-tert-butyloxycarbonyl (Boc) group using a protic acid such as HCl in a solvent such as MeOH gives S32. Chiral preparative SFC or HPLC separation may be utilized to provide two or more single stereoisomers of compounds derived from formula S32.

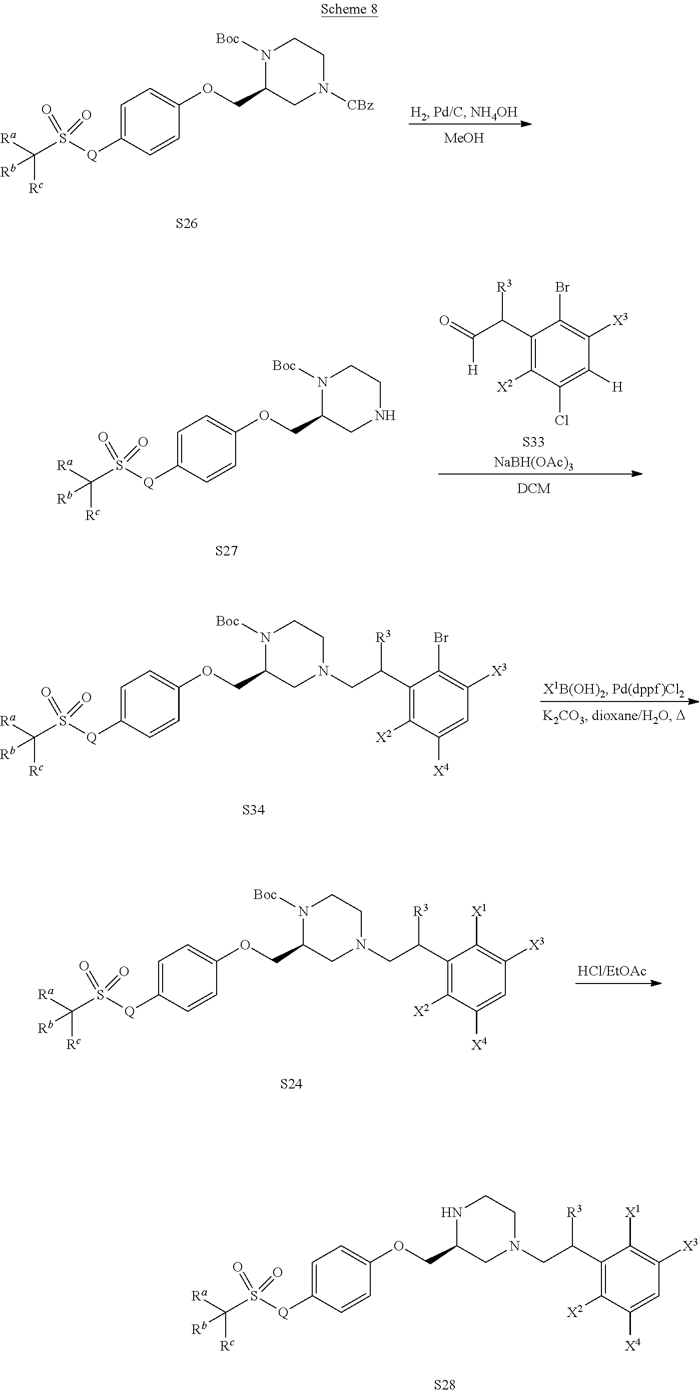

A synthetic approach to compounds of formula S28 is depicted in Scheme 8. Selective removal of the benzyl carbamate of S26 via hydrogenation gives piperazine S27. Reductive amination under the action of sodium triacetoxyborohydride generates S34. Further manipulation may be accomplished by Suzuki coupling with a boronic acid (X¹B(OH₂)), Pd(dppf)Cl₂ catalyst, and potassium carbonate to give S24. Removal of the N-tert-butyloxycarbonyl (Boc) group upon reaction with HCl in EtOAc gives compounds of formula S28. Chiral preparative SFC or HPLC separation may be utilized to provide two or more single stereoisomers of compounds derived from formula S28.

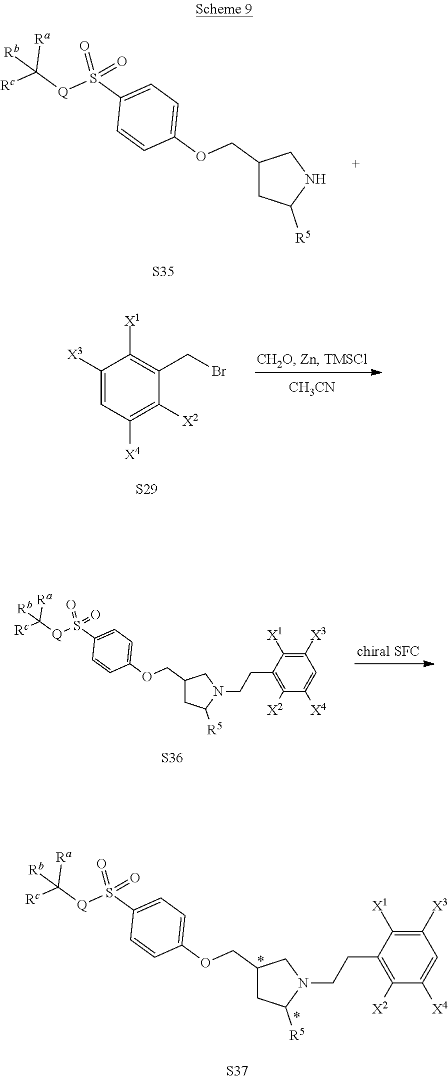

Scheme 9 depicts an alternate application of the three-component coupling described in Scheme 7. Coupling of pyrrolidine S35, benzyl bromide S29, and formaldehyde provides compounds of formula S36. Chiral preparative SFC or HPLC separation may be utilized to provide two or more single stereoisomers of formula S37.

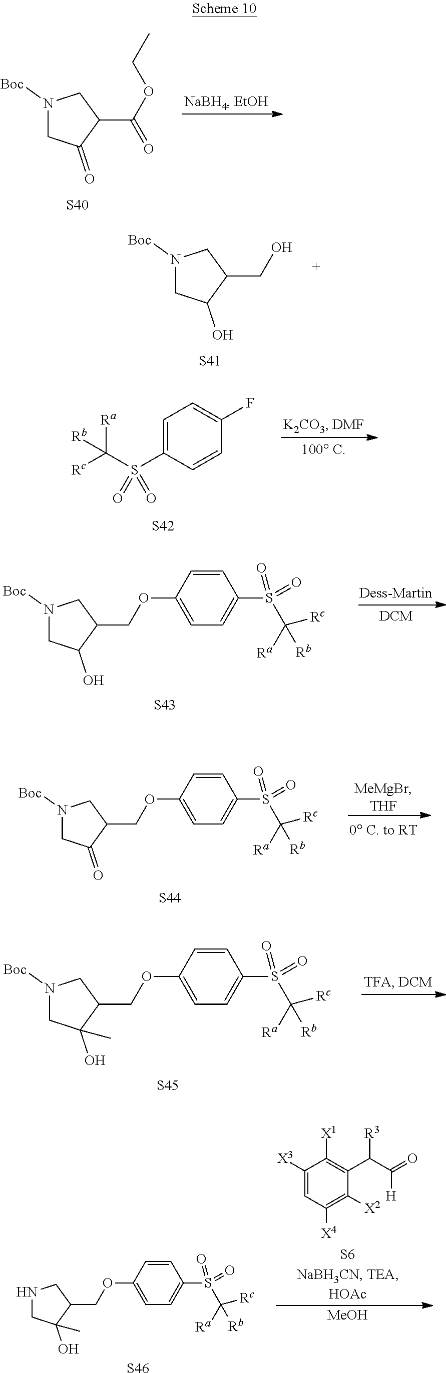

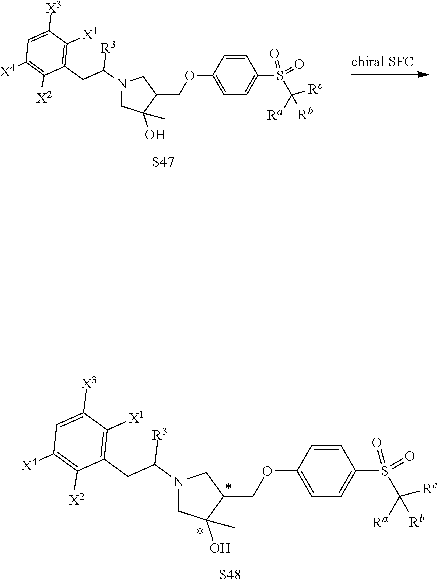

Scheme 10 depicts an alternative approach to pyrrolidine analogs of formula S48. NaBH₄ reduction of j-keto ester S40 gives diol S41, which can undergo S_{N}Ar reaction with fluorobenzene S42 upon heating in DMF with potassium carbonate as base. Oxidation to ketone S44 may be achieved with the Dess-Martin periodinane. Reaction with an alkyl metal reagent such as methylmagnesium bromide gives tertiary alcohol S45, as a mixture of isomers. Removal of the N-tert-butyloxycarbonyl (Boc) group with a protic acid such as TFA, followed by reductive amination with aldehyde S6, gives pyrrolidine S47. Chiral preparative SFC or HPLC separation may be utilized to provide two or more single stereoisomers of formula S48.

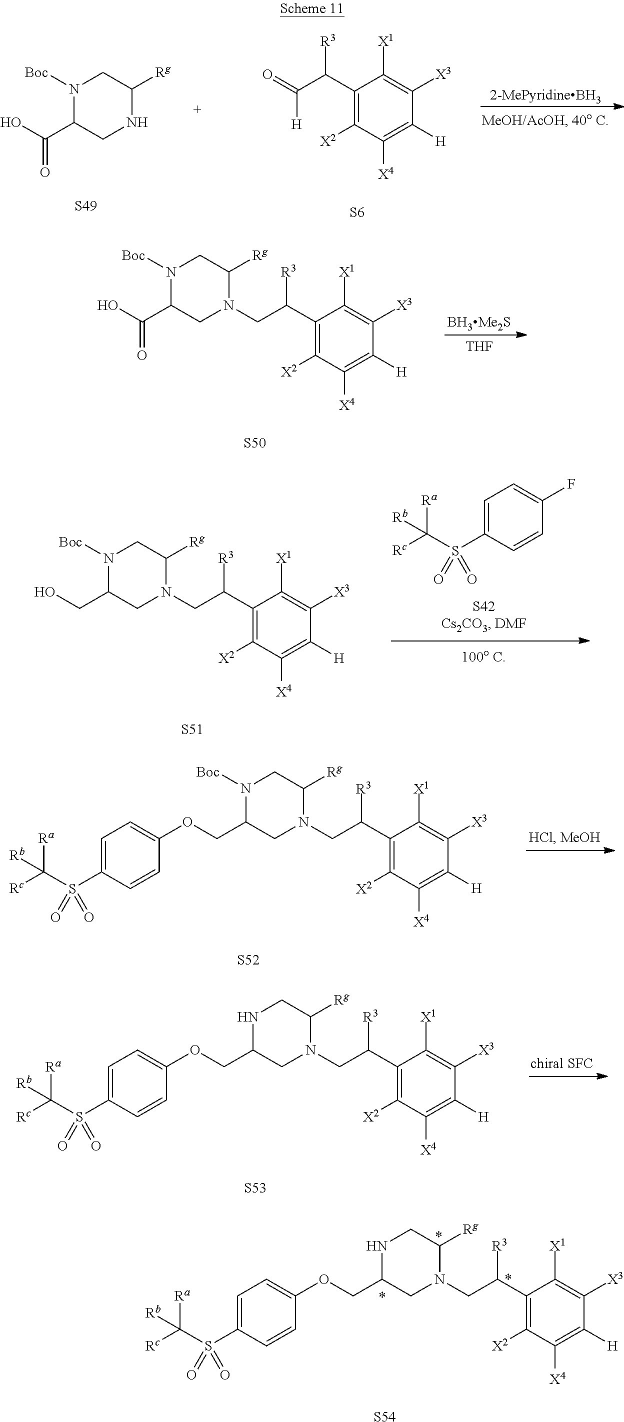

Scheme 11 depicts a route to analogs bearing substituted piperazine cores. Reductive amination of piperazine S49 and aldehyde S6 with 2-methyl pyridine borane complex gives S50. Reduction of the carboxylic acid S50 with borane-dimethylsulfide gives alcohol S51, which may undergo S_{N}Ar reaction with fluorobenzene S42 to give S52. Removal of the N-tert-butyloxycarbonyl (Boc) group with a protic acid such as HCl in a solvent such as MeOH gives piperazine S53. Chiral preparative SFC or HPLC separation may be utilized to provide two or more single stereoisomers of formula S54.

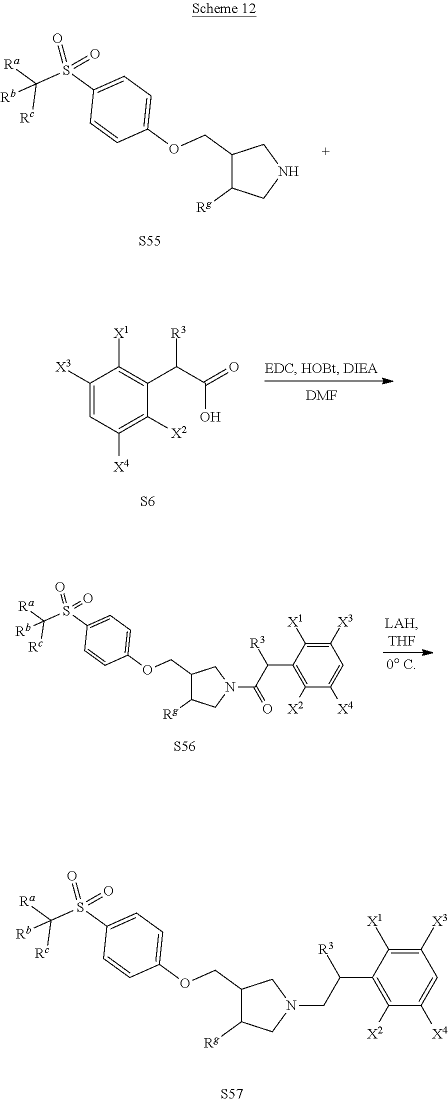

Scheme 11 depicts an approach to compounds of formula S57. Amide bond formation between an amine such as pyrrolidine S55 and carboxylic acid S6 using EDC and HOBt with a tertiary amine base such as DIEA to provide S56. Amide reduction can be achieved upon treatment with lithium aluminum hydride to give compounds of formula S57. Chiral preparative SFC or HPLC separation may be utilized to provide two or more single stereoisomers of compounds derived from formula S57.

### General Procedure for Intermediate A

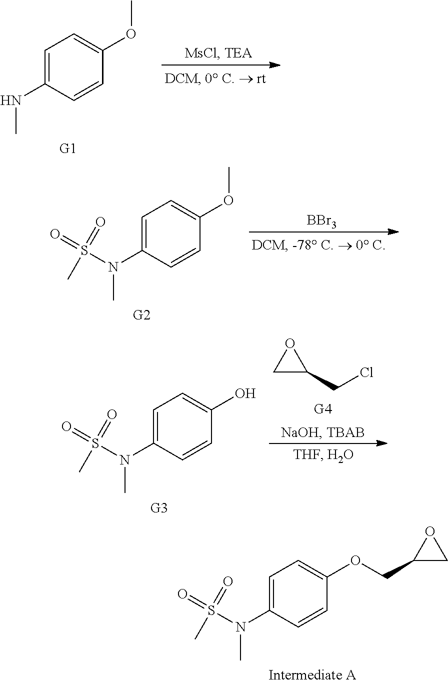

### Step 1: N-(4-methoxyphenyl)-N-methylmethanesulfonamide

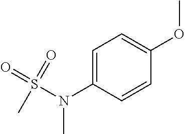

To a solution of 4-methoxy-N-methyl-aniline (G1, 7.00 g, 51.0 mmol) in DCM (60 mL) was added TEA (14.2 mL, 102 mmol). After cooling the reaction to 0° C., MsCl (5.13 mL, 66.3 mmol) was added dropwise. The mixture was stirred at room temperature for 1 h. The reaction mixture was cooled to 0° C. and quenched by the addition of water (40 mL). The biphasic mixture was extracted with DCM (60 mL×2). The combined organic layers were washed with brine (40 mL), dried over Na₂SO₄, filtered and concentrated in vacuo to give N-(4-methoxyphenyl)-N-methylmethanesulfonamide (G2), which was used in the next step without further purification. MS=216.1 [M+H]⁺.

### Step 2: N-(4-hydroxyphenyl)-N-methylmethanesulfonamide

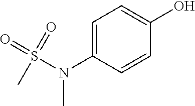

To a −78° C. solution of N-(4-methoxyphenyl)-N-methylmethanesulfonamide (G2, 11.4 g, 53.0 mmol) in DCM (100 mL) was added BBr₃ (10.2 mL, 106 mmol) dropwise. The mixture was warmed to 0° C. and stirred for 2 h. The reaction mixture was quenched by the dropwise addition of water (70 mL), and the mixture was stirred at room temperature for 20 min. Then the mixture was extracted with DCM (100 mL×3). The combined organic layers were washed with brine (80 mL), dried over Na₂SO₄, filtered and concentrated under in vacuo to give N-(4-hydroxyphenyl)-N-methyl-methanesulfonamide (G3). MS=202.2 [M+H]⁺.

### Step 3: (S)—N-methyl-N-(4-(oxiran-2-ylmethoxy)phenyl)methanesulfonamide (Intermediate A)

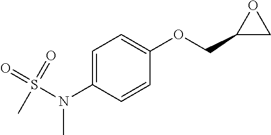 [SMILES: CN(c1ccc(OC[C@@H]2CO2)cc1)S(C)(=O)=O]

To a solution of N-(4-hydroxyphenyl)-N-methyl-methanesulfonamide (G3, 4.00 g, 19.9 mmol) and (R)-2-(chloromethyl)oxirane (G4, 3.12 mL, 39.8 mmol) in water (10 mL) and THF (20 mL) was added TBAB (897 mg, 2.78 mmol). Next, a solution of NaOH (1.19 g, 29.8 mmol) in water (10 mL) was added dropwise. The mixture was stirred at room temperature for 12 h. The reaction mixture was diluted by the dropwise addition of water (20 mL), and then extracted with EtOAc (20 mL×3). The combined organic layers were washed with brine (15 mL), dried over Na₂SO₄, filtered and concentrated in vacuo. The crude residue was purified by normal phase silica gel chromatography (Biotage 40 g cartridge, 0-55% EtOAc in petroleum ether). The resulting residue was further purified by re-crystallization (50 mL of 1:10 EtOAc in petroleum ether) to give (S)—N-methyl-N-(4-(oxiran-2-ylmethoxy)phenyl)methanesulfonamide (Intermediate A). MS 280.0 [M+Na]⁺.

The following intermediates in Table 2 were prepared according to procedures similar to those described for Intermediate A using the appropriate starting materials.

TABLE 2

| | | | Exact Mass |
|---|---|---|---|
| Intermediate | Structure | IUPAC Name | [M + H]⁺ |
| B | 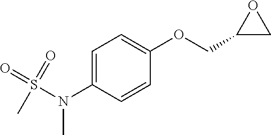 [SMILES: CN(c1ccc(OC[C@H]2CO2)cc1)S(C)(=O)=O] | (R)-N-methyl-N-(4- (oxiran-2- ylmethoxy)phenyl) methanesulfonamide | Calc’d 280.1 Found 280.0 [M + Na]⁺ |
| C | 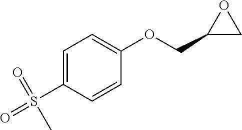 | (S)-2-((4- (methylsulfonyl)phenoxy) methyl)oxirane | Calc’d 229.1 Found 229.0 |
| D | 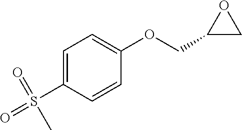 | (R)-2-((4- (methylsulfonyl)phenoxy) methyl)oxirane | Calc’d 229.1 Found 229.2 |
| E | 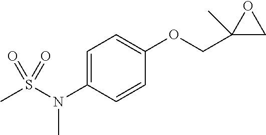 [SMILES: CN(c1ccc(OCC2(C)CO2)cc1)S(C)(=O)=O] | N-methyl-N-(4-((2- methyloxiran-2- yl)methoxy)phenyl) methanesulfonamide | Calc’d 193.1 Found 193.1 [M − CH₃O₂S + H]⁺ |
| F | 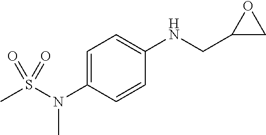 [SMILES: CN(c1ccc(NCC2CO2)cc1)S(C)(=O)=O] | N-methyl-N-(4-((oxiran-2- ylmethyl)amino)phenyl) methanesulfonamide | Calc’d 279.1 Found 279.0 [M + Na]⁺ |

### General Procedure for Intermediate G

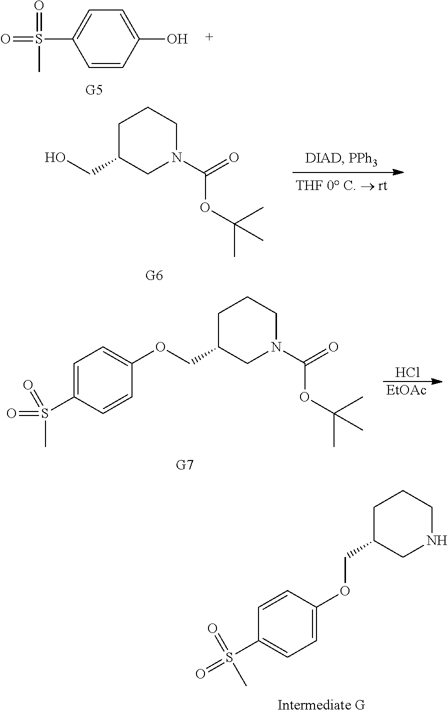

### Step 1: (tert-butyl (R)-3-((4-(methylsulfonyl)phenoxy)methyl)piperidine-1-carboxylate

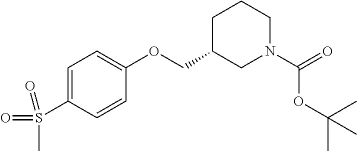 [SMILES: CC(C)(C)OC(=O)N1CCC[C@@H](COc2ccc(S(C)(=O)=O)cc2)C1]

To a solution of tert-butyl (R)-3-(hydroxymethyl)piperidine-1-carboxylate (G6, 1.00 g, 4.64 mmol) and 4-methylsulfonylphenol (G5, 800 mg, 4.64 mmol) in THF (20 mL) was added PPh₃ (2.44 g, 9.29 mmol). The mixture was cooled to 0° C. and DIAD (1.88 g, 9.29 mmol, 1.81 mL, 2.00 eq) was added dropwise. After stirring at room temperature for 12 h, the reaction mixture was concentrated in vacuo. The residue was purified by normal phase silica gel chromatography (Biotage 12 g cartridge, 1-25% EtOAc in petroleum ether) to give (tert-butyl (R)-3-((4-(methylsulfonyl)phenoxy)methyl)piperidine-1-carboxylate (G7). MS=387.2 [M+NH₄]⁺.

### Step 2: (R)-3-((4-(methylsulfonyl)phenoxy)methyl)piperidine

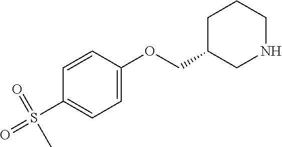 [SMILES: CS(=O)(=O)c1ccc(OC[C@@H]2CCCNC2)cc1]

To a solution of (tert-butyl (R)-3-((4-(methylsulfonyl)phenoxy)methyl)piperidine-1-carboxylate (7, 780 mg, 2.11 mmol, 1.00 eq) in EtOAc (5 mL) was added HCl/EtOAc (4 M, 10 mL). The mixture was stirred at room temperature for 2 h. The suspension was triturated with MTBE (100 mL) and the resulting solid was isolated through filtration to give (R)-3-((4-(methylsulfonyl)phenoxy)methyl)piperidine (Intermediate G) which was used in the next step without further purification. MS=270.1 [M+H]⁺.

The following intermediate in Table 3 was prepared according to procedures similar to those described for Intermediate G using the appropriate starting materials.

TABLE 3

| | | | Exact Mass |
|---|---|---|---|
| Intermediate | Structure | IUPAC Name | [M + H]⁺ |
| N | 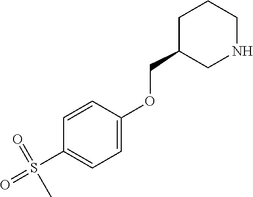 [SMILES: CS(=O)(=O)c1ccc(OC[C@H]2CCCNC2)cc1] | (S)-3-((4- (methylsulfonyl)phenoxy) methyl)piperidine | Calc’d 270.1 Found 270.1 |

1

### General Procedure for Intermediate I

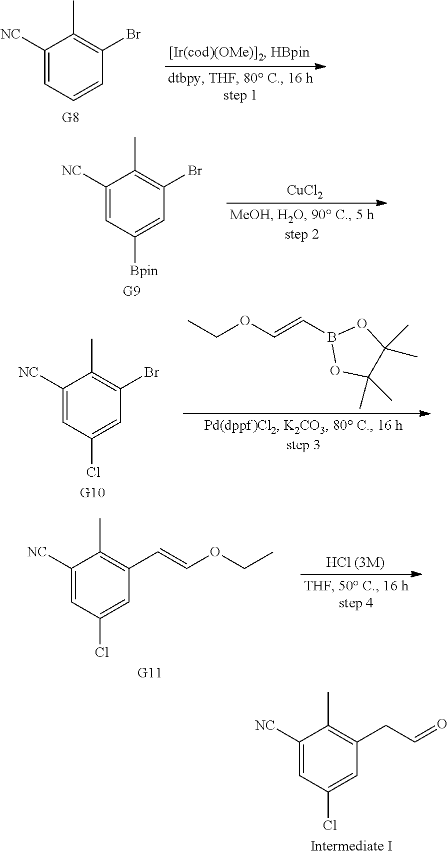

### Step 1: 3-bromo-2-methyl-5-(4,4,5,5-tetramethyl-1,3,2-dioxaborolan-2-yl)benzonitrile

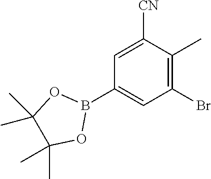

To a solution of 3-bromo-2-methyl-benzonitrile (G8, 5.00 g, 25.5 mmol) and 4,4,5,5-tetramethyl-1,3,2-dioxaborolane (32.6 g, 255 mmol) in THF (80 mL) under N₂ was added 4-tert-butyl-2-(4-tert-butyl-2-pyridyl)pyridine (685 mg, 2.55 mmol) and [Ir(cod)(OMe)]₂ (845 mg, 1.28 mmol). Then the mixture was stirred at 80° C. for 8 h under N₂. The reaction mixture was diluted with H₂O (100 mL) and extracted with EtOAc (100 mL×3). The combined organic layers were washed with brine (100 mL), dried over Na₂SO₄, filtered and concentrated under reduced pressure. The residue was purified by flash silica gel chromatography (ISCO 80 g cartridge, 0-20% EtOAc:Hexane) to give 3-bromo-2-methyl-5-(4,4,5,5-tetramethyl-1,3,2-dioxaborolan-2-yl)benzonitrile (G9). ¹H NMR (400 MHz, CDCl₃): δ 8.15 (s, 1H), 7.98 (s, 1H), 2.65 (s, 3H), 1.35 (s, 12H).

### Step 2: 3-bromo-5-chloro-2-methylbenzonitrile

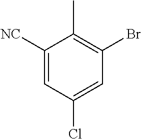

To solution of 3-bromo-2-methyl-5-(4,4,5,5-tetramethyl-1,3,2-dioxaborolan-2-yl)benzonitrile (G9, 3.10 g, 9.63 mmol) in MeOH (30 mL) and H₂O (10 mL) was added CuCl₂ (3.88 g, 28.9 mmol) at room temperature, then the mixture was stirred at 90° C. for 16 h. After cooling to room temperature, the reaction mixture was diluted with H₂O (50 mL) and extracted with EtOAc (30 mL×3). The combined organic layers were washed with brine (20 mL), dried over Na₂SO₄, filtered and concentrated under reduced pressure to give a residue. The residue was purified by flash silica gel chromatography (ISCO 20 g cartridge, 0-5% EtOAc:Hexane) to give 3-bromo-5-chloro-2-methyl-benzonitrile (G10). ¹H NMR (400 MHz, CDCl₃): δ 7.78 (s, 1H), 7.57 (s, 1H), 2.61 (s, 3H).

### Step 3: (E)-5-chloro-3-(2-ethoxyvinyl)-2-methylbenzonitrile

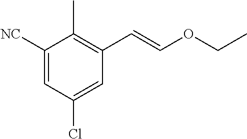

To a solution of 3-bromo-5-chloro-2-methyl-benzonitrile (G10, 300 mg, 1.30 mmol) and 2-[(E)-2-ethoxyvinyl]-4,4,5,5-tetramethyl-1,3,2-dioxaborolane (296 mg, 1.50 mmol) in 1,4-dioxane (5 mL) under N₂ were added K₂CO₃ (360 mg, 2.60 mmol) and Pd(dppf)Cl₂ (95.2 mg, 130 μmol). Then the mixture was stirred at 80° C. for 16 h. The reaction mixture was allowed to cool to room temperature, diluted with H₂O (10 mL), and extracted with EtOAc (10 mL×3). The combined organic layers were washed with brine (20 mL), dried over Na₂SO₄, filtered and concentrated under reduced pressure to give a residue. The residue was purified by flash silica gel chromatography (ISCO 4 g cartridge, 0-5% EtOAc:Hexane) to give 5-chloro-3-[(E)-2-ethoxyvinyl]-2-methyl-benzonitrile (G11). MS=222.1 [M+H]⁺.

### Step 4: 5-chloro-2-methyl-3-(2-oxoethyl)benzonitrile (Intermediate I)

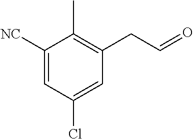

To a solution of 5-chloro-3-[(E)-2-ethoxyvinyl]-2-methyl-benzonitrile (G11, 1.50 g, 6.77 mmol) in THF (12 mL) was added aqueous HCl (4 M, 12 mL), and then the mixture was stirred at 50° C. for 5 h. The reaction mixture was diluted with H₂O (30 mL) and extracted with EtOAc (10 mL×3). The combined organic layers were washed with brine (30 mL), dried over Na₂SO₄, filtered and concentrated under reduced pressure to give a residue. The residue was purified by flash silica gel chromatography (ISCO 12 g cartridge, 0-50% EtOAc:Hexane) to give 5-chloro-2-methyl-3-(2-oxoethyl)benzonitrile (Intermediate I). MS=194.0 [M+H]⁺.

### General Procedure for Intermediate J

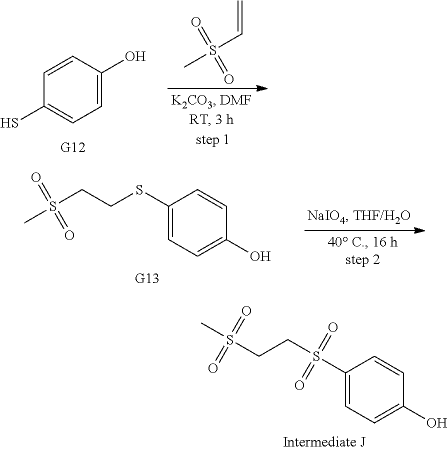

### Step 1: 4-((2-(methylsulfonyl)ethyl)thio)phenol

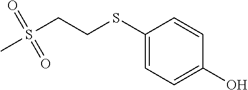

To a solution of 4-sulfanylphenol (G12, 4.75 g, 37.7 mmol) and 1-methylsulfonylethylene (4 g, 37.7 mmol) in DMF (40 mL) was added K₂CO₃ (7.81 g, 56.5 mmol). The mixture was stirred at room temperature for 3 h. The reaction mixture was diluted with H₂O (100 mL) and then extracted with EtOAc (100 mL×3). The combined organic layers were washed with brine (100 mL), dried over Na₂SO₄, filtered and concentrated under reduced pressure to give a residue. The residue was purified by flash silica gel chromatography (Biotage 40 g cartridge, 0-40% EtOAc:Hexane) to give 4-((2-(methylsulfonyl) ethyl) thio) phenol (G13). MS=231.1 [M−H]⁻.

### Step 2: 4-((2-(methylsulfonyl)ethyl)sulfonyl)phenol (Intermediate J)

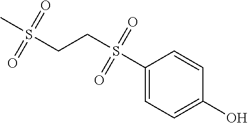 [SMILES: CS(=O)(=O)CCS(=O)(=O)c1ccc(O)cc1]

To a solution of 4-(2-methylsulfonylethylsulfanyl) phenol (G13, 3.00 g, 12.9 mmol) in THF (40 mL) at room temperature was added a mixture of NaIO₄ (5.50 g, 25.8 mmol) in H₂O (10 mL). The mixture was then stirred at 40° C. for 16 h. The reaction mixture was diluted with saturated aqueous Na₂SO₃ (20 mL), extracted with EtOAc (20 mL×3), dried over Na₂SO₄, filtered and concentrated under reduced pressure to give a residue. The residue was purified by flash silica gel chromatography (ISCO 40 g cartridge, 0-100% EtOAc:Hexane) to give 4-(2-methylsulfonylethylsulfonyl) phenol (Intermediate J). MS=263.1 [M−H]⁻.

### General Procedure for Intermediate K

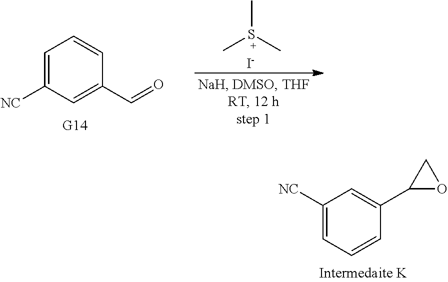

### Step 1: 3-(oxiran-2-yl)benzonitrile (Intermediate K)

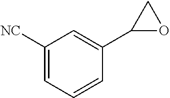 [SMILES: N#Cc1cccc(C2CO2)c1]

To a suspension of NaH (73 mg, 1.83 mmol, 60% by weight in mineral oil) in THF (5 mL) was added a solution of trimethylsulfonium iodide (374 mg, 1.83 mmol) in DMSO (3 mL) under N₂. After 10 min, a solution of 3-formylbenzonitrile (G14, 200 mg, 1.53 mmol) in THF (2 mL) was slowly added. The reaction mixture was degassed and purged with N₂ for 3 times, and then the mixture was stirred at room temperature for 12 h under N₂. The reaction mixture was quenched by addition of H₂O (10 mL) and extracted with EtOAc (10 mL×3). The combined organic layers were washed with brine (10 mL×3), dried over Na₂SO₄, filtered and concentrated under reduced pressure to give a residue. The residue was purified by flash silica gel chromatography (Biotage 4 g cartridge, 0-50% EtOAc:Hexane) to give 3-(oxiran-2-yl)benzonitrile (Intermediate K). MS=146.1 [M+H]⁺.

### General Procedure for Intermediate M

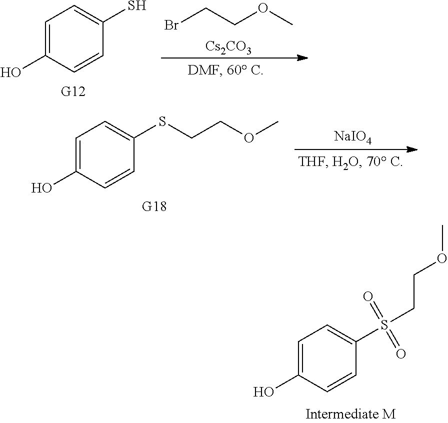

### Step 1: 4-((2-methoxyethyl)thio)phenol

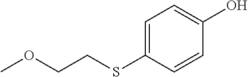

A mixture of 4-sulfanylphenol (G12, 1 g, 7.93 mmol), 1-bromo-2-methoxy-ethane (1.10 g, 7.93 mmol) and Cs₂CO₃ (2.58 g, 7.93 mmol) in DMF (12 mL) was stirred at 60° C. for 3 h. The reaction mixture was poured into ice water (30 mL) and extracted with EtOAc (30 mL×3). The combined organic layers were washed with brine (50 mL), dried over Na₂SO₄, filtered and concentrated under reduced pressure. The residue was purified by flash silica gel chromatography (Biotage 20 g cartridge, 0-20% EtOAc:Hexane) to give 4-(2-methoxyethylsulfanyl)phenol (G18). MS=183.1 [M−H]⁻.

### Step 2: 4-((2-methoxyethyl)sulfonyl)phenol (Intermediate M)

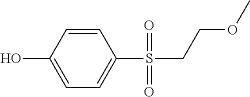

A mixture of 4-(2-methoxyethylsulfanyl)phenol (G18, 700 mg, 3.80 mmol) and NaIO₄ (2.44 g, 11.4 mmol) in THF (6 mL) and H₂O (6 mL) was stirred at 70° C. for 16 h. The mixture was filtered, quenched with saturated aqueous Na₂S₂O₃ (15 mL) and extracted with EtOAc (20 mL×3). The combined organic layers were washed with brine (40 mL), dried over Na₂SO₄, filtered and the filtrate was concentrated under reduced pressure. The residue was purified by flash silica gel chromatography (Biotage 20 g cartridge, 0-70% EtOAc:Hexane) to give 4-(2-methoxyethylsulfonyl)phenol (Intermediate M). MS=217.1 [M+H]⁺.

The following intermediate in Table 4 was prepared according to procedures similar to those described for Intermediate M using the appropriate starting materials.

TABLE 4

| Intermediate | Structure | IUPAC Name | Exact Mass [M-H] |
|---|---|---|---|
| N | 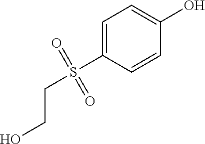 [SMILES: O=S(=O)(CCO)c1ccc(O)cc1] | 4-(2-hydroxy ethylsulfonyl)phenol | Calc’d 201.1 Found 201.1 |

### General Procedure for Intermediate O and Intermediate P

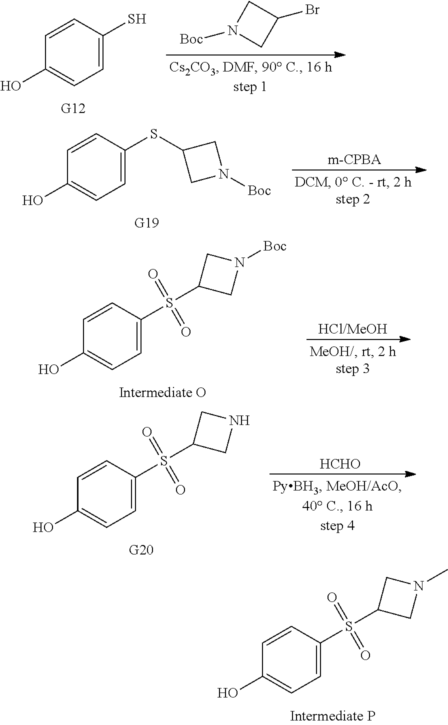

### Step 1: tert-butyl 3-((4-hydroxyphenyl)thio)azetidine-1-carboxylate

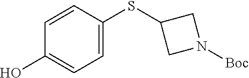 [SMILES: CC(C)(C)OC(=O)N1CC(Sc2ccc(O)cc2)C1]

To a solution of tert-butyl 3-bromoazetidine-1-carboxylate (1.87 g, 7.93 mmol) in DMF (30 mL) at room temperature were added Cs₂CO₃ (5.16 g, 15.85 mmol) and 4-sulfanylphenol (G12, 1.00 g, 7.93 mmol), and the resulting mixture was stirred at 90° C. for 16 h. The reaction mixture was poured into water (30 mL) and extracted with EtOAc (30 mL×3). The combined organic layers were washed with brine (50 mL), dried over Na₂SO₄, filtered and concentrated under reduced pressure to give a residue. The residue was purified by flash silica gel chromatography (Biotage 12 g cartridge, 0-100% EtOAc:hexane) to give tert-butyl 3-(4-hydroxyphenyl)sulfanylazetidine-1-carboxylate (G19). MS=282.1 [M+H]⁺.

### Step 2: tert-butyl 3-((4-hydroxyphenyl)sulfonyl)azetidine-1-carboxylate (Intermediate O)

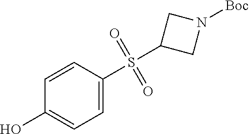 [SMILES: CC(C)(C)OC(=O)N1CC(S(=O)(=O)c2ccc(O)cc2)C1]

To a solution of tert-butyl 3-(4-hydroxyphenyl)sulfanylazetidine-1-carboxylate (G19, 50 mg, 178 μmol) in DCM (2 mL) at 0° C. was added m-CPBA (46 mg, 267 μmol, 77% purity) at 0° C. The mixture was stirred at room temperature for 2 h. The reaction mixture was quenched by addition saturated aqueous Na₂SO₃ (5 mL), and then extracted with EtOAc (5 mL×3). The combined organic layers were washed with brine (5 mL×3), dried over Na₂SO₄, filtered and concentrated under reduced pressure to give a residue. The residue was purified by flash silica gel chromatography (Biotage 4 g cartridge, 0-50% EtOAc:Hexane) to give tert-butyl 3-(4-hydroxyphenyl)sulfonylazetidine-1-carboxylate (Intermediate O). MS=258.0 [M-C₄H₈+H]⁺.

### Step 3: 4-(azetidin-3-ylsulfonyl)phenol

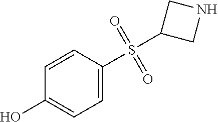 [SMILES: O=S(=O)(c1ccc(O)cc1)C1CNC1]

To a solution of tert-butyl 3-(4-hydroxyphenyl)sulfonylazetidine-1-carboxylate (Intermediate O, 0.5 g, 1.91 mmol) in MeOH (3 mL) was added HCl/MeOH (4 M, 3 mL). The reaction mixture was stirred at room temperature for 2 h. The reaction mixture was then concentrated under reduced pressure to give 4-(azetidin-3-ylsulfonyl)phenol HCl salt (G20), which was taken to the next step without further purification. MS=214.1 [M+H]⁺.

### Step 4: 4-((1-methylazetidin-3-yl)sulfonyl)phenol (Intermediate P)

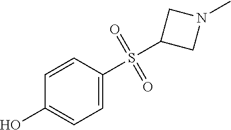

To a solution of 4-(187zetidine-3-ylsulfonyl)phenol (G20, 400 mg, 1.60 mmol, HCl salt) in MeOH (5 mL) and AcOH (0.05 mL) were added HCHO (120 mg, 4.00 mmol) and borane-2-methylpyridine complex (205 mg, 1.90 mmol). The mixture was stirred at 40° C. for 16 h. The reaction mixture was quenched by addition of saturated aqueous NaHCO₃ (10 mL), and then extracted with EtOAc (10 mL×3). The combined organic layers were washed with brine (10 mL), dried over Na₂SO₄, filtered and concentrated under reduced pressure to give a residue. The residue was purified by flash silica gel chromatography (Biotage 12 g cartridge, 0-100% EtOAc:Hexane) to give 4-(1-methylazetidin-3-yl)sulfonylphenol (Intermediate P). MS=228.0 [M+H]⁺.

### General Procedure for Intermediate Q

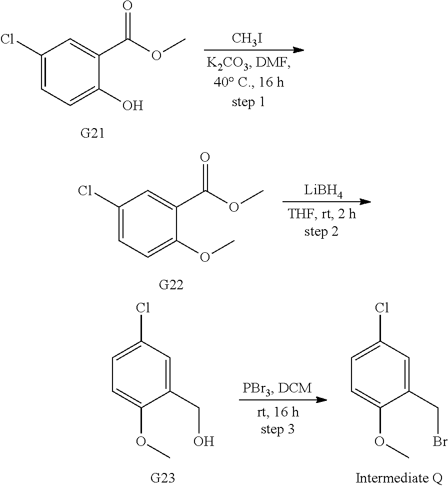

### Step 1: methyl 5-chloro-2-methoxybenzoate

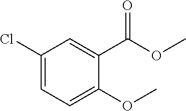

To a solution of methyl 5-chloro-2-hydroxy-benzoate (G21, 2.00 g, 10.7 mmol) in DMF (20 mL) were added K₂CO₃ (2.96 g, 21.4 mmol) and CH₃I (7.61 g, 53.6 mmol). The mixture was stirred at 40° C. for 16 h. The reaction mixture was then cooled to room temperature, poured into water (100 mL), and extracted with EtOAc (30 mL×3). The combined organic layers were washed with brine (80 mL), dried over Na₂SO₄, filtered and concentrated under reduced pressure to give the residue, which was purified by flash silica gel chromatography (ISCO 20 g cartridge, 0-10% EtOAc:Hexane) to give methyl 5-chloro-2-methoxy-benzoate (G22). MS=201.0 [M+H]⁺.

### Step 2: (5-chloro-2-methoxyphenyl)methanol

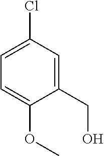

To a solution of methyl 5-chloro-2-methoxy-benzoate (G22, 1.50 g, 7.48 mmol) in THF (15 mL) was added LiBH₄ (2 M in THF, 7.5 mL, 15.0 mmol) at 0° C. The reaction mixture was stirred at room temperature for 2 h. The reaction was quenched with water (20 mL) and extracted with EtOAc (20 mL×3). The combined organic layers were washed with brine (60 mL), dried over Na₂SO₄, filtered and concentrated to give a residue. The residue was purified by flash silica gel chromatography (ISCO 20 g cartridge, 0-50% EtOAc:Hexane) to give (5-chloro-2-methoxy-phenyl)methanol (G23). ¹H NMR (400 MHz, CDCl₃): δ 7.30 (d, J=2.8 Hz, 1H), 7.25-7.21 (m, 1H), 6.81 (d, J=8.8 Hz, 1H), 4.66 (s, 2H), 3.86 (s, 3H).

### Step 3: 2-(bromomethyl)-4-chloro-1-methoxybenzene (Intermediate Q)

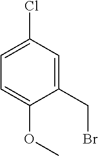

To a solution of (5-chloro-2-methoxy-phenyl)methanol (G23, 1.10 g, 6.37 mmol) in DCM (50 mL) at 0° C. was added PBr₃ (1.73 g, 6.37 mmol). The reaction mixture was then stirred at room temperature for 16 h. The mixture was concentrated, diluted with water (50 mL) and neutralized by addition of saturated aqueous NaHCO₃ to pH=7-8, then extracted with DCM (50 mL×3). The combined organic layers were washed with brine (100 mL), dried over Na₂SO₄, filtered and concentrated to give 2-(bromomethyl)-4-chloro-1-methoxy-benzene (Intermediate Q). ¹H NMR (400 MHz, CDCl₃): δ 7.27 (d, J=2.8 Hz, 1H), 7.21 (dd, J=5.6 Hz, 2.8 Hz, 1H), 6.77 (d, J=8.8 Hz, 1H), 4.45 (s, 2H), 3.85 (s, 3H).

### General Procedure for Intermediate R

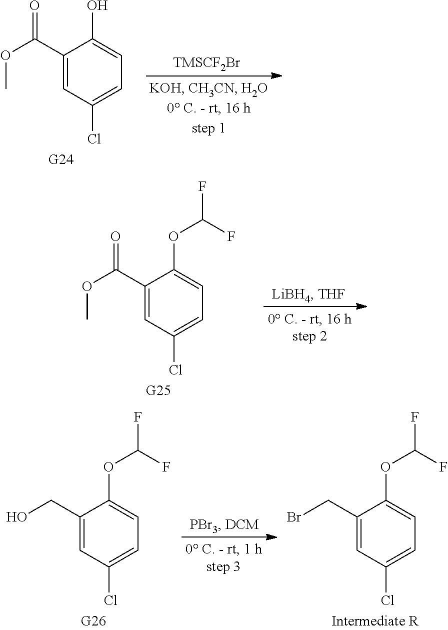

### Step 1: methyl 5-chloro-2-(difluoromethoxy)benzoate

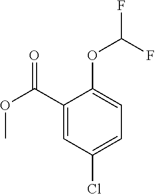

To a mixture of methyl 5-chloro-2-hydroxy-benzoate (G24, 1.50 g, 8.04 mmol,) in MeCN (75 mL) and H₂O (37.5 mL) were added KOH (2.71 g, 48.2 mmol) and [bromo(difluoro)methyl]-trimethylsilane (3.27 g, 16.1 mmol) at 0° C. The mixture was then stirred at room temperature for 16 h. The reaction mixture was poured into water (20 mL) and extracted with EtOAc (20 mL×3). The combined organic layers were washed with brine (25 mL), dried over Na₂SO₄, filtered and concentrated under reduced pressure to give a residue. The residue was purified by flash silica gel chromatography (ISCO 20 g cartridge, 0-20% EtOAc:Hexane) to give methyl 5-chloro-2-(difluoromethoxy)benzoate (G25). ¹H NMR (400 MHz, DMSO-d₆): δ 7.86 (d, J=2.8 Hz, 1H), 7.75 (dd, J=8.8 Hz, 2.8 Hz, 1H), 7.37 (d, J=9.2 Hz, 1H), 7.20 (t, J=73.6 Hz, 1H), 7.21-7.03 (m, 1H), 3.85 (s, 3H).

### Step 2: (5-chloro-2-(difluoromethoxy)phenyl)methanol

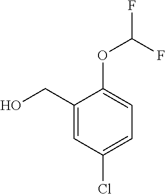 [SMILES: OCc1cc(Cl)ccc1OC(F)F]

To a mixture of methyl 5-chloro-2-(difluoromethoxy)benzoate (G25, 200 mg, 845 μmol) in THF (5 mL) at 0° C. was added LiBH₄ (4 M in THF, 634 μL, 2.54 mmol) dropwise under N₂, the mixture was then stirred at room temperature for 16 h under N₂. The reaction mixture was poured into saturated aqueous NH₄Cl (5 mL), then extracted with EtOAc (15 mL×3). The combined organic layers were washed with brine (5 mL), dried over anhydrous Na₂SO₄, filtered and concentrated under reduced pressure to give [5-chloro-2-(difluoromethoxy)phenyl]methanol (G26), which was used without further purification. MS=190.9 [M−H₂O+H]⁺.

### Step 3: 2-(bromomethyl)-4-chloro-1-(difluoromethoxy) benzene (Intermediate R)

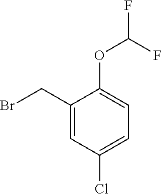 [SMILES: FC(F)Oc1ccc(Cl)cc1CBr]

To a solution of [5-chloro-2-(difluoromethoxy)phenyl]methanol (G26, 170 mg, 815 μmol) in DCM (3 mL) was added PBr₃ (221 mg, 815 μmol) at 0° C. The mixture was stirred at room temperature for 1 h. The reaction mixture was then poured into water (15 mL) and extracted with DCM (10 mL×3). The combined organic layers were washed with brine (5 mL), dried over anhydrous Na₂SO₄, filtered and concentrated under reduced pressure to give a residue. The residue was purified by flash silica gel chromatography (ISCO 4 g cartridge, 0-10% EtOAc:Hexane) to give 2-(bromomethyl)-4-chloro-1-(difluoromethoxy)benzene (Intermediate R). ¹H NMR (400 MHz, DMSO-d₆): δ 7.69 (d, J=2.4 Hz, 1H), 7.50 (dd, J=8.0 Hz, 2.8 Hz, 1H), 7.25 (d, J=8.8 Hz, 1H), 7.31 (t, J=73.2 Hz, 1H), 4.63 (s, 2H).

### General Procedure for Intermediate S

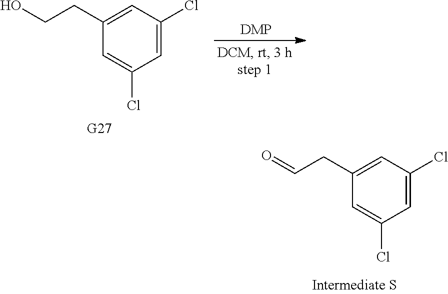 [SMILES: OCCc1cc(Cl)cc(Cl)c1 ; O=CCc1cc(Cl)cc(Cl)c1]

### Step 1: 2-(3, 5-dichlorophenyl) acetaldehyde (Intermediate S)

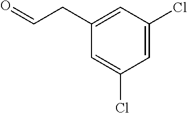 [SMILES: O=CCc1cc(Cl)cc(Cl)c1]

To a mixture of 2-(3,5-dichlorophenyl)ethanol (G27, 1.00 g, 5.23 mmol) in DCM (10 mL) was added DMP (2.66 g, 6.28 mmol). The mixture was then stirred at room temperature for 3 h under N₂. The reaction mixture was diluted with H₂O (5 mL) and extracted with EtOAc (8 mL×3). The combined organic layers were washed with brine (10 mL), dried over Na₂SO₄, filtered and concentrated under reduced pressure to give a residue. The residue was purified by flash silica gel chromatography (Biotage 12 g cartridge, 0-20% EtOAc:Hexane) to give 2-(3, 5-dichlorophenyl) acetaldehyde (Intermediate S). ¹H NMR (400 MHz, DMSO-d₆): δ 9.68 (s, 1H), 7.53 (s, 1H), 7.47 (s, 1H), 7.46 (s, 1H), 3.86 (s, 2H).

The following intermediate in Table 5 was prepared according to procedures similar to those described for Intermediate S using the appropriate starting materials.

TABLE 5

| | | | ¹H NMR (400 MHZ, |
|---|---|---|---|
| Intermediate | Structure | IUPAC Name | DMSO-d₆): |
| T | 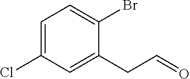 [SMILES: O=CCc1cc(Cl)ccc1Br] | 2-(2-bromo-5-chloro- phenyl)acetaldehyde | δ 9.70 (s, 1H), 7.66 (d, J = 8.8 Hz, 1H), 7.51 (d, J = 2.4 Hz, 1H), 7.32 (d, J = 8.4 Hz, 2.4 Hz, 1H), 3.98 (s, 2H). |
| U | 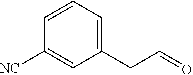 [SMILES: N#Cc1cccc(CC=O)c1] | 3-(2-oxoethyl) benzonitrile | δ 9.80 (t, J = 2.0 Hz, 1H), 7.63-7.52 (m, 4H), 3.92- 3.88 (m, 2H). |

### General Procedure for Intermediate V

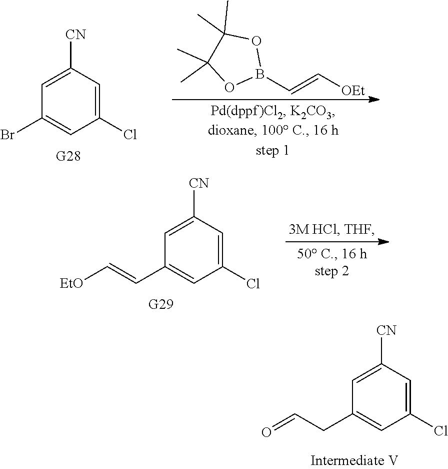

### Step 1: (E)-3-chloro-5-(2-ethoxyvinyl) benzonitrile

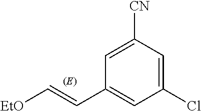 [SMILES: CCO/C=C/c1cc(Cl)cc(C#N)c1]

To a solution of 2-[(E)-2-ethoxyvinyl]-4,4,5,5-tetramethyl-1,3,2-dioxaborolane (503 mg, 2.54 mmol) and 3-bromo-5-chloro-benzonitrile (G28, 500 mg, 2.31 mmol) in dioxane (10 mL) were added K₂CO₃ (958 mg, 6.93 mmol) and Pd(dppf)Cl₂ (169 mg, 231 μmol) under N₂. The reaction mixture was then stirred at 100° C. for 16 h under N₂. The reaction mixture was diluted with H₂O (10 mL) and extracted with EtOAc (5 mL×3). The combined organic layers were washed with brine (10 mL), dried over Na₂SO₄, filtered and concentrated under reduced pressure to give (E)-3-chloro-5-(2-ethoxyvinyl)benzonitrile (G29), which was taken to the next step without further purification. ¹H NMR (400 MHz, CDCl₃): δ 7.40 (s, 1H), 7.36 (s, 1H), 7.35 (s, 1H), 7.05 (d, J=13.2 Hz, 1H), 5.73 (d, J=12.8 Hz, 1H), 3.94 (q, J=7.2 Hz, 2H), 1.37 (t, J=7.2 Hz, 3H).

### Step 2: 3-chloro-5-(2-oxoethyl) benzonitrile (Intermediate V)

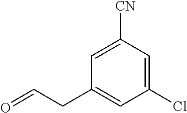 [SMILES: N#Cc1cc(Cl)cc(CC=O)c1]

To a solution of (E)-3-chloro-5-(2-ethoxyvinyl)benzonitrile (G29, 750 mg, 3.61 mmol) in THF (4 mL) was added HCl (3 M in H₂O, 4 mL). The reaction mixture was then stirred at 50° C. for 16 h. The reaction mixture was diluted with H₂O (10 mL) and extracted with EtOAc (5 mL×3). The combined organic layers were washed with brine (10 mL), dried over Na₂SO₄, filtered and concentrated under reduced pressure to give a residue. The residue was purified by flash silica gel chromatography (Biotage 4 g cartridge, 0-30% EtOAc:Hexane) to give 3-chloro-5-(2-oxoethyl)benzonitrile (Intermediate V). ¹H NMR (400 MHz, CDCl₃): δ 9.81 (s, 1H), 7.60 (s, 1H), 7.46 (s, 1H), 7.42 (s, 1H), 3.80 (s, 2H).

The following intermediate in Table 6 was prepared according to procedures similar to those described for Intermediate V using the appropriate starting materials.

TABLE 6

| | | | ¹H NMR (400 MHZ, |
|---|---|---|---|
| Intermediate | Structure | IUPAC Name | CDCl₃): |
| W | 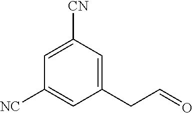 [SMILES: N#Cc1cc(C#N)cc(CC=O)c1] | 5-(2-oxoethyl) isophthalonitrile | δ 9.86 (s, 1H), 7.90 (s, 1H), 7.81 (s, 1H), 7.74 (s, 1H), 3.49 (s, 2H). |

### General Procedure for Intermediate X

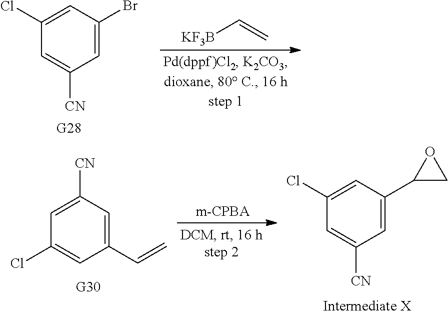

### Step 1: 3-chloro-5-vinylbenzonitrile

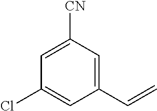

To a solution of 3-bromo-5-chloro-benzonitrile (G28, 2.50 g, 11.5 mmol), potassium vinyltrifluoroborate (3.09 g, 23.1 mmol) in dioxane (25 mL) and H₂O (2.5 mL) were added K₂CO₃ (3.19 g, 23.1 mmol) and Pd(dppf)Cl₂ (845 mg, 1.15 mmol). The mixture was purged with N₂ three times and then stirred at 80° C. for 16 h. The reaction mixture was poured into water (50 mL) and extracted with EtOAc (40 mL×3). The combined organic layers were washed with brine (100 mL), dried over Na₂SO₄, filtered and concentrated under reduced pressure to give a residue. The residue was purified by flash silica gel chromatography (BIOTAGE 20 g cartridge, 0-10% EtOAc:Hexane) to give 3-chloro-5-vinyl-benzonitrile (G30).

¹H NMR (400 MHz, CDCl₃): δ 7.60 (s, 1H), 7.56 (s, 1H), 7.52 (s, 1H), 6.69-6.62 (m, 1H), 5.85 (d, J=17.6 Hz, 1H), 5.47 (d, J=10.8 Hz, 1H).

### Step 2: 3-chloro-5-(oxiran-2-yl) benzonitrile (Intermediate X)

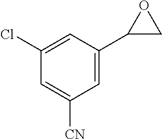 [SMILES: N#Cc1cc(Cl)cc(C2CO2)c1]

To a solution of 3-chloro-5-vinyl-benzonitrile (G30, 500 mg, 3.06 mmol) in DCM (5 mL) was added mCPBA (931 mg, 4.58 mmol, 85% purity) at 0° C., the reaction was stirred at room temperature for 16 h. The reaction mixture was then poured into saturated aqueous Na₂SO₃ (20 mL) and extracted with EtOAc (30 mL×3). The combined organic layers were washed with brine (120 mL), dried over Na₂SO₄, filtered and concentrated under reduced pressure to give a residue. The residue was purified by flash silica gel chromatography (ISCO 12 g cartridge, 0-100% EtOAc:Hexane) to give 3-chloro-5-(oxiran-2-yl)benzonitrile (Intermediate X).The following intermediate in Table 7 was prepared according to procedures similar to those described for Intermediate X using the appropriate starting materials.

TABLE 7

| | | | ¹H NMR (400 MHZ, |
|---|---|---|---|
| Intermediate | Structure | IUPAC Name | CDCl₃): |
| Y | 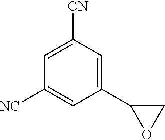 [SMILES: N#Cc1cc(C#N)cc(C2CO2)c1] | 5-(oxiran-2-yl) benzene-1, 3-dicarbonitrile | δ 7.69 (s, 1H), 7.62-7.61 (m, 2H), 3.77-3.75 (m, 1H), 3.07-3.05 (m, 1H), 2.58-2.56 (m, 1H). |

### General Procedure for Intermediate Z

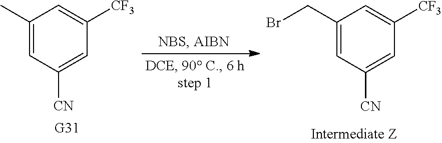

### Step 1: 3-(bromomethyl)-5-(trifluoromethyl) benzonitrile (Intermediate Z)

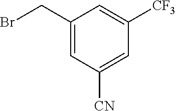 [SMILES: N#Cc1cc(CBr)cc(C(F)(F)F)c1]

A mixture of 3-methyl-5-(trifluoromethyl) benzonitrile (G31, 750 mg, 4.05 mmol), NBS (865 mg, 4.86 mmol) and AIBN (67 mg, 405 μmol) in DCE (7.5 mL) was stirred at 90° C. for 6 h. The mixture was then diluted with DCM (20 mL) and washed with water (20 mL). The organic layer was concentrated under reduced pressure. The residue was purified twice by flash silica gel chromatography (Biotage 12 g cartridge, 0-1% EtOAc:Hexane) to give 3-(bromomethyl)-5-(trifluoro methyl)benzonitrile (Intermediate Z). MS=264.0/266.1 [M+H]⁺.

### General Procedure for Intermediate AA

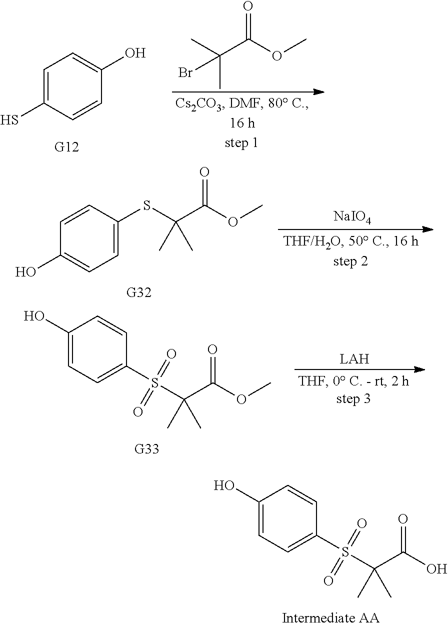

### Step 1: methyl 2-((4-hydroxyphenyl)thio)-2-methylpropanoate

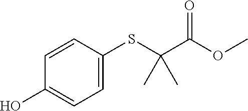

To a mixture of 4-sulfanylphenol (G12, 2.00 g, 15.8 mmol) and methyl 2-bromo-2-methyl-propanoate (2.40 g, 13.3 mmol) in DMF (30 mL) was added Cs₂CO₃ (7.20 g, 22.1 mmol). The mixture was then stirred at 80° C. for 16 h. The reaction mixture was diluted with saturated aqueous NH₄Cl (30 mL), extracted with EtOAc (30 mL×3), dried over Na₂SO₄, filtered and concentrated under reduced pressure to give a residue. The residue was purified by flash silica gel chromatography (ISCO 12 g cartridge, 0-100% EtOAc:Hexane) to give methyl 2-(4-hydroxyphenyl) sulfanyl-2-methyl-propanoate (G32). MS=225.2 [M−H]⁻.

### Step 2: methyl 2-((4-hydroxyphenyl) sulfonyl)-2-methylpropanoate

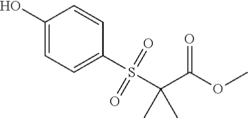 [SMILES: COC(=O)C(C)(C)S(=O)(=O)c1ccc(O)cc1]

To a solution of methyl 2-(4-hydroxyphenyl) sulfanyl-2-methyl-propanoate (G32, 2.30 g, 10.2 mmol) in THF (30 mL) was added a solution of NaIO₄ (6.52 g, 30.5 mmol) in H₂O (6 mL). The mixture was stirred at 50° C. for 16 h. The reaction mixture was quenched with saturated aqueous Na₂SO₃ (15 mL), extracted with EtOAc (10 mL×3), dried over Na₂SO₄, filtered and concentrated under reduced pressure to give a residue. The residue was purified by flash silica gel chromatography (ISCO 20 g cartridge, 0-30% EtOAc:Hexane) to give methyl 2-(4-hydroxyphenyl) sulfonyl-2-methyl-propanoate (G33). MS=257.1 [M−H]⁻.

### Step 3: 4-((1-hydroxy-2-methylpropan-2-yl) sulfonyl)phenol

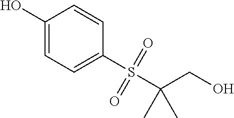

To a solution of methyl 2-(4-hydroxyphenyl) sulfonyl-2-methyl-propanoate (G33, 2.30 g, 8.90 mmol) in THF (30 mL) at 0° C. was added LiAlH₄ (507 mg, 13.4 mmol). The mixture was stirred at room temperature for 2 h. The residue was diluted with aqueous NaOH (5 M, 2 mL), dried over Na₂SO₄, filtered and concentrated under reduced pressure to give a residue. The residue was purified by flash silica gel chromatography (ISCO 4 g cartridge, 0-100% EtOAc:Hexane to give 4-(2-hydroxy-1, 1-dimethyl-ethyl) sulfonylphenol (Intermediate AA). MS=229.0 [M−H]⁻.

### General Procedure for Intermediate AB

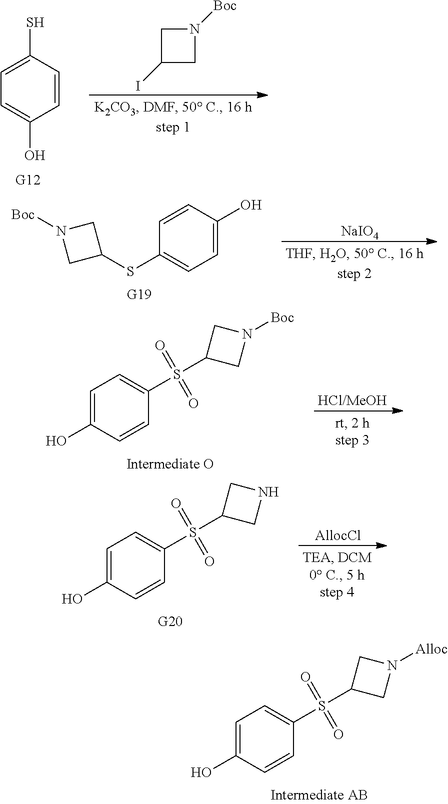

### Step 1: tert-butyl 3-((4-hydroxyphenyl) thio) azetidine-1-carboxylate

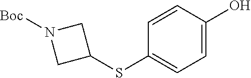 [SMILES: CC(C)(C)OC(=O)N1CC(Sc2ccc(O)cc2)C1]

To a solution of 4-sulfanylphenol (G12, 10.0 g, 79.2 mmol) in DMF (100 mL) were added K₂CO₃ (10.9 g, 79.2 mmol) and tert-butyl 3-iodoazetidine-1-carboxylate (22.4 g, 79.2 mmol). The mixture was then stirred at 40° C. for 16 h. The reaction mixture was diluted with water (200 mL) and extracted with EtOAc (100 mL×3). The combined organic layers were washed with H₂O (80 mL) and brine (80 mL), dried over (Na₂SO₄), filtered and concentrated under reduced pressure to give a residue. The crude product was triturated with MTBE (100 mL) at room temperature for 5 h and filtered to give tert-butyl 3-(4-hydroxyphenyl)sulfanylazetidine-1-carboxylate (G19). MS=226.1 [M-C₄H₈+H]⁺.

### Step 2: tert-butyl 3-((4-hydroxyphenyl) sulfonyl) azetidine-1-carboxylate

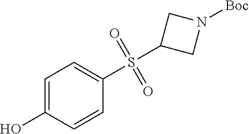 [SMILES: CC(C)(C)OC(=O)N1CC(S(=O)(=O)c2ccc(O)cc2)C1]

To a solution of tert-butyl 3-(4-hydroxyphenyl) sulfanylazetidine-1-carboxylate (G19, 6.50 g, 13.9 mmol) in THF (60 mL) and H₂O (20 mL) was added NaIO₄ (8.80 g, 41.6 mmol,). The mixture was stirred at 50° C. for 16 h. The reaction mixture was cooled to room temperature, quenched with saturated aqueous Na₂SO₃ (60 mL) and extracted with EtOAc (30 mL×3). The combined organic layers were washed with brine (30 mL), dried over Na₂SO₄, filtered and concentrated under reduced pressure to give a residue. The residue was purified by flash silica gel chromatography (ISCO 20 g cartridge, 0-100% EtOAc:Hexane) to give tert-butyl 3-(4-hydroxyphenyl) sulfonylazetidine-1-carboxylate (Intermediate O). MS=312.2 [M−H]⁻.

### Step 3: 4-(azetidin-3-ylsulfonyl) phenol

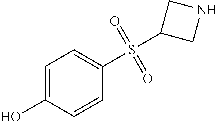 [SMILES: O=S(=O)(c1ccc(O)cc1)C1CNC1]

To a solution of tert-butyl 3-(4-hydroxyphenyl) sulfonylazetidine-1-carboxylate (Intermediate O, 1.5 g, 4.79 mmol) in MeOH (4 mL) was added HCl/MeOH (4 M, 12 mL). The mixture was stirred at room temperature for 2 h. The reaction mixture was concentrated under reduced pressure to give 4-(azetidin-3-ylsulfonyl) phenol HCl salt (G20), which was used without further purification. MS=214.1 [M+H]⁺.

### Step 4: allyl 3-((4-hydroxyphenyl) sulfonyl) azetidine-1-carboxylate (Intermediate AB)

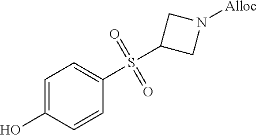

To a solution of 4-(azetidin-3-ylsulfonyl)phenol (G20, 1.1 g, 4.41 mmol, HCl salt) in DCM (10 mL) were added TEA (1.11 g, 11.0 mmol, 1.53 mL) and allyl chloroformate (584 mg, 4.85 mmol) at 0° C. The mixture was then stirred at room temperature for 3 h. The reaction mixture was diluted with H₂O (30 mL) and extracted with DCM (15 mL×3). The combined organic layers were washed with brine (5 mL), dried over Na₂SO₄, filtered and concentrated under reduced pressure to give a residue. The residue was purified by flash silica gel chromatography (ISCO 12 g cartridge, 0-100% EtOAc:Hexane) to give allyl 3-(4-hydroxyphenyl)sulfonylazetidine-1-carboxylate (Intermediate AB). MS=298.1 [M+H]⁺.

### General Procedure for Intermediate AC

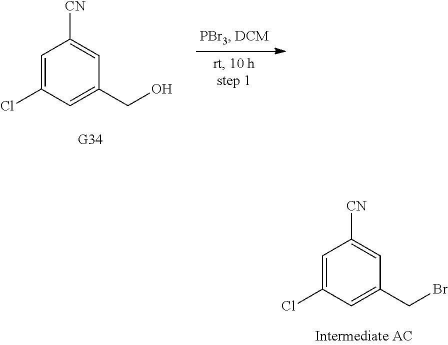 [SMILES: N#Cc1cc(Cl)cc(CO)c1 ; N#Cc1cc(Cl)cc(CBr)c1]

### Step 1: 3-(bromomethyl)-5-chlorobenzonitrile (Intermediate AC)

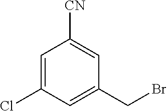 [SMILES: N#Cc1cc(Cl)cc(CBr)c1]

To a solution of 3-chloro-5-(hydroxymethyl)benzonitrile (G34, 500 mg, 2.98 mmol) in DCM (10 mL) was added PBr₃ (807 mg, 2.98 mmol) at 0° C. The reaction mixture was then stirred at room temperature for 10 h, then the reaction mixture was concentrated. The residue was diluted with water (10 mL) and neutralized by addition of saturated aqueous NaHCO₃ to pH=7, then extracted with EtOAc (10 mL×3). The combined organic layers were washed with brine (20 mL), dried over Na₂SO₄, filtered and concentrated to give 3-(bromomethyl)-5-chloro-benzonitrile (Intermediate AC), which was taken to the next step without further purification. ¹H NMR (400 MHz, CDCl₃): δ 7.63 (s, 1H), 7.58 (s, 2H), 4.43 (s, 2H).

### General Procedure for Intermediate AD

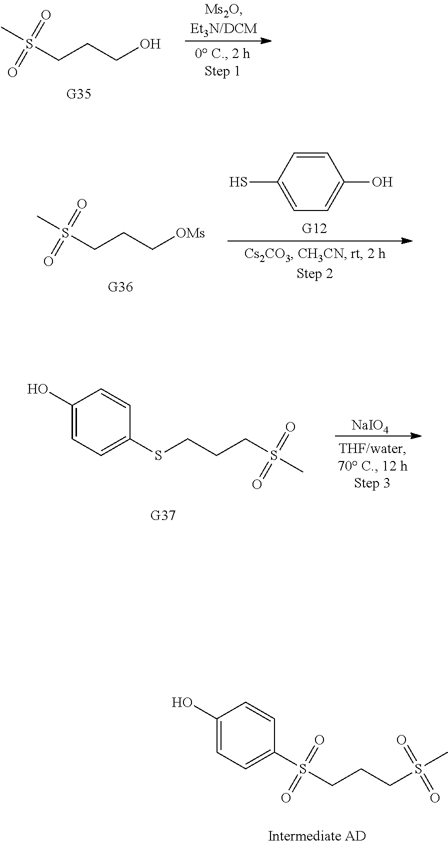

### Step 1: 3-(methylsulfonyl)propyl methanesulfonate

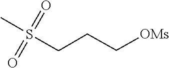

To a solution of 3-methylsulfonylpropan-1-ol (G35, 500 mg, 3.62 mmol) in DCM (5 mL) at 0° C. were added Et₃N (732 mg, 7.24 mmol) and methylsulfonyl methanesulfonate (945 mg, 5.43 mmol). The mixture was stirred at 0° C. for 2 h. The reaction mixture was diluted with H₂O (20 mL) and extracted with EtOAc (10 mL×3). The combined organic layers were washed with brine (30 mL), dried over Na₂SO₄, filtered and concentrated under reduced pressure to give crude 3-(methylsulfonyl)propyl methanesulfonate (G36), which was taken to the next step without further purification. ¹H NMR (400 MHz, DMSO-d₆): δ 4.30 (t, J=6.4 Hz, 2H), 3.23-3.01 (m, 5H), 3.01 (s, 3H), 2.13-2.08 (m, 2H).

### Step 2: 4-((3-(methylsulfonyl)propyl)thio)phenol

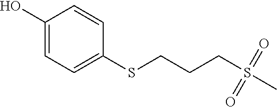

To a mixture of 3-(methylsulfonyl)propyl methanesulfonate (G36, 390 mg, 1.80 mmol) and 4-sulfanylphenol (G12, 318 mg, 2.52 mmol) in CH₃CN (5 mL) was added Cs₂CO₃ (705 mg, 2.16 mmol). The mixture was then stirred at room temperature for 2 h. The reaction mixture was then diluted with H₂O (20 mL) and extracted with EtOAc (20 mL×3). The combined organic layers were washed with brine (40 mL), dried over Na₂SO₄, filtered and concentrated under reduced pressure to give a residue. The residue was purified by flash silica gel chromatography (ISCO 12 g cartridge, 0-50% EtOAc:Hexane) to give 4-(3-methylsulfonylpropylsulfanyl)phenol (G37). MS=245.1 [M−H]⁻.

### Step 3: 4-((3-(methylsulfonyl)propyl)sulfonyl)phenol (Intermediate AD)

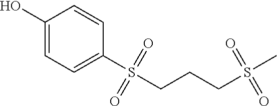 [SMILES: CS(=O)(=O)CCCS(=O)(=O)c1ccc(O)cc1]

To a solution of 4-(3-methylsulfonylpropylsulfanyl)phenol (G37, 370 mg, 1.50 mmol) in THF (2 mL) and H₂O (2 mL) was added NaIO₄ (964 mg, 4.51 mmol) at 0° C. The mixture was then stirred at 70° C. for 12 h. The reaction mixture was quenched by addition of saturated aqueous Na₂SO₃ (10 mL), and then diluted with H₂O (5 mL) and extracted with EtOAc (15 mL×3). The combined organic layers were washed with brine (30 mL), dried over Na₂SO₄, filtered and concentrated under reduced pressure to give a residue. The residue was purified by flash silica gel chromatography (ISCO 12 g cartridge, 0-50% EtOAc:Hexane) to give 4-(3-methylsulfonylpropylsulfonyl)phenol (Intermediate AD). MS=277.1 [M−H]⁻.

### General Procedure for Intermediate AE

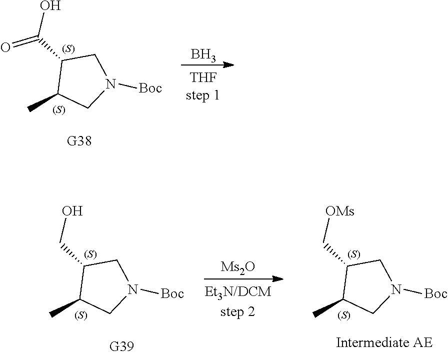

### Step 1: tert-butyl (3S,4S)-3-(hydroxymethyl)-4-methylpyrrolidine-1-carboxylate

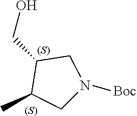

To a solution of (3S,4S)-1-tert-butoxycarbonyl-4-methyl-pyrrolidine-3-carboxylic acid (G38, 5.00 g, 21.8 mmol) in THF (50 mL) was added BH₃ (10 M in Me₂S, 10.9 mL, 109 mmol) at 0° C. Then the reaction was stirred at room temperature for 16 h. The reaction mixture was quenched by addition of MeOH (40 mL) and stirred 0.5 h. The mixture was then diluted with water (100 mL) and extracted with EtOAc (60 mL×3). The combined organic layers were washed with brine (100 mL), dried over Na₂SO₄, filtered and the filtrate was concentrated to give tert-butyl (3S,4S)-3-(hydroxymethyl)-4-methyl-pyrrolidine-1-carboxylate (G39). MS=160.2 [M-C₄H₈+H]⁺.

### Step 2: tert-butyl (3S,4S)-3-methyl-4-(((methylsulfonyl)oxy)methyl)pyrrolidine-1-carboxylate (Intermediate AE)

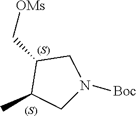

To a solution of tert-butyl (3S,4S)-3-(hydroxymethyl)-4-methyl-pyrrolidine-1-carboxylate (G39, 200 mg, 929 μmol) in DCM (3 mL) were added Et₃N (188 mg, 1.86 mmol) and methylsulfonyl methanesulfonate (161 mg, 929 μmol) at 0° C. The mixture was stirred at room temperature for 2 h. The reaction mixture was then concentrated under reduced pressure. The residue was diluted with saturated aqueous NH₄Cl (5 mL), extracted with EtOAc (5 mL×3), dried over Na2ₛO₄, filtered and concentrated under reduced pressure to give tert-butyl (3S,4S)-3-methyl-4-(((methylsulfonyl)oxy)methyl)pyrrolidine-1-carboxylate (Intermediate AE). ¹H NMR (400 MHz, CDCl₃): δ 4.32-4.28 (m, 1H), 4.16-4.14 (m, 1H), 3.65-3.61 (m, 3H), 3.20-3.18 (m, 1H), 3.03 (s, 3H), 2.97-2.95 (m, 1H), 2.33-2.20 (m, 4H), 1.46 (s, 9H).

The following intermediate in Table 8 was prepared according to procedures similar to those described for Intermediate AE using the appropriate starting materials.

TABLE 8

| | | | ¹H NMR (400 MHZ, |
|---|---|---|---|
| Intermediate | Structure | IUPAC Name | DMSO-d₆): |
| AF | 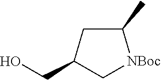 | tert-butyl (2R,4S)-2- methyl-4- (methylsulfonyloxymethyl) pyrrolidine-1-carboxylate | δ 4.24-4.14 (m, 2H), 3.73- 3.71 (m, 1H), 3.63-3.58 (m, 1H), 3.18 (s, 3H), 2.96 2.92 (m, 1H), 2.25-2.23 (m, 1H), 2.21-2.18 (m, 1H), 1.39 (s, 9H), 1.32- 1.27 (m, 1H), 1.86 (d, J = 3.2 Hz, 3H). |

In the following Examples, the numbers in the column headed with “No.” in each of Tables 9-25 refer to the corresponding compound numbers in Table 1.

### Example 1

### 1-(3-chlorophenethyl)-3-((4-(methylsulfonyl)phenoxy)methyl)azepane (Compound 1)

### (R) or (S)-1-(3-chlorophenethyl)-3-((4-(methylsulfonyl)phenoxy)methyl)azepane (Compound 2)

### (S) or (R)-1-(3-chlorophenethyl)-3-((4-(methylsulfonyl)phenoxy)methyl)azepane (Compound 3)

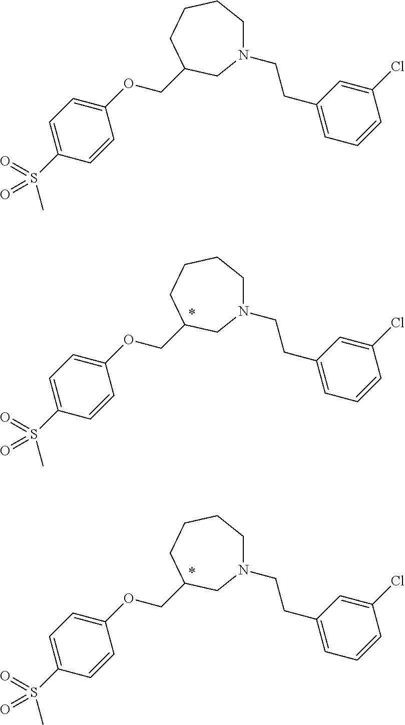 [SMILES: CS(=O)(=O)c1ccc(OCC2CCCCN(CCc3cccc(Cl)c3)C2)cc1 ; CS(=O)(=O)c1ccc(OCC2CCCCN(CCc3cccc(Cl)c3)C2)cc1 ; CS(=O)(=O)c1ccc(OCC2CCCCN(CCc3cccc(Cl)c3)C2)cc1]

### Step 1: tert-butyl 3-((4-(methylsulfonyl)phenoxy)methyl)azepane-1-carboxylate

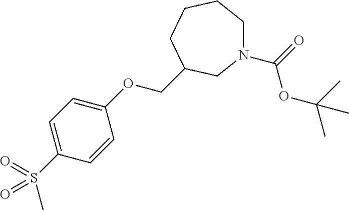 [SMILES: CC(C)(C)OC(=O)N1CCCCC(COc2ccc(S(C)(=O)=O)cc2)C1]

To a mixture of tert-butyl-3-(hydroxymethyl)azepane-1-carboxylate (200 mg, 0.872 mmol), 4-(methylsulfonyl)phenol (195 mg, 1.13 mmol), and PPh₃ (297 mg, 1.13 mmol) in THF (5 mL) at 0° C. was added DIAD (509 μL, 2.62 mmol) dropwise. The mixture was stirred at room temperature for 12 h. The reaction mixture was cooled to 0° C., quenched by the addition of water (20 mL), then extracted with EtOAc (25 mL×2). The combined organic layers were washed with brine (20 mL), dried over Na₂SO₄, filtered and concentrated in vacuo. The crude residue was purified by normal phase silica gel chromatography (Biotage 10 g cartridge, 0-85% EtOAc in petroleum ether) to give tert-butyl 3-((4-(methylsulfonyl)phenoxy)methyl)azepane-1-carboxylate. MS=384.2 [M+H]⁺.

### Step 2: 3-((4-(methylsulfonyl)phenoxy)methyl)azepane

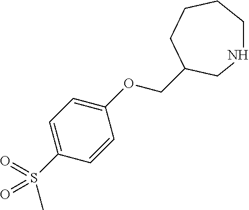 [SMILES: CS(=O)(=O)c1ccc(OCC2CCCCNC2)cc1]

To a solution of tert-butyl 3-((4-(methylsulfonyl)phenoxy)methyl)azepane-1-carboxylate (260 mg, 0.678 mmol) in EtOAc (2 mL) was added HCl/EtOAc (4 M, 2 mL), the mixture was stirred at room temperature for 1 h. The reaction mixture was concentrated in vacuo to give 3-((4-(methylsulfonyl)phenoxy)methyl)azepane (HCl salt), which was used in the next step without further purification. MS=284.2 [M+H]⁺.

### Step 3: 1-(3-chlorophenethyl)-3-((4-(methylsulfonyl)phenoxy)methyl)azepane (Compound 1)

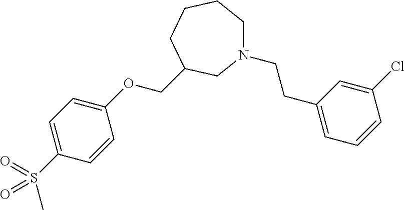 [SMILES: CS(=O)(=O)c1ccc(OCC2CCCCN(CCc3cccc(Cl)c3)C2)cc1]

A mixture of 3-((4-(methylsulfonyl)phenoxy)methyl)azepane (200 mg, 0.625 mmol, HCl salt), 2-(3-chlorophenyl)acetaldehyde (142 mg, 0.918 mmol) in MeOH (4 mL) was stirred at room temperature for 1 h, then NaBH₃CN (66.5 mg, 1.06 mmol) was added. The resulting mixture was stirred at room temperature for 1 h. The reaction mixture was quenched by addition of water (1 mL) and concentrated in vacuo. The crude residue was purified by reverse phase preparative HPLC (Waters Xbridge BEH C18 column, 45-80% MeCN/10 mM NH₄HCO₃ in water) to give 1-(3-chlorophenethyl)-3-((4-(methylsulfonyl)phenoxy)methyl)azepane (Compound 1). ¹H NMR (400 MHz, DMSO-d₆): δ 7.82 (d, J=8.8 Hz, 2H), 7.28-7.22 (m, 3H), 7.14-7.11 (m, 3H), 3.89-3.82 (m, 2H), 3.14 (s, 3H), 2.77-2.70 (m, 1H), 2.70-2.60 (m, 7H), 2.06-2.05 (m, 1H), 1.72-1.51 (m, 5H), 1.35-1.33 (m, 1H). MS=422.3 [M+H]⁺.

### Step 4: (R) or (S)-1-(3-chlorophenethyl)-3-((4-(methylsulfonyl)phenoxy)methyl)azepane (Compound 2); (S) or (R)-1-(3-chlorophenethyl)-3-((4-(methylsulfonyl)phenoxy)methyl)azepane (Compound 3)

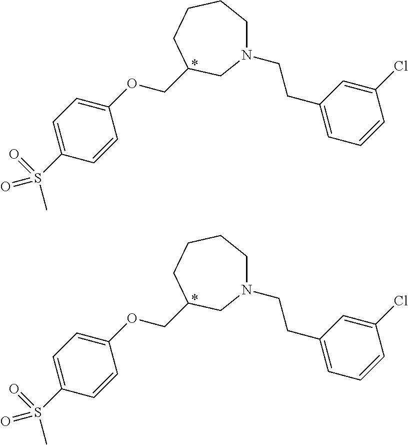 [SMILES: CS(=O)(=O)c1ccc(OCC2CCCCN(CCc3cccc(Cl)c3)C2)cc1 ; CS(=O)(=O)c1ccc(OCC2CCCCN(CCc3cccc(Cl)c3)C2)cc1]

1-(3-chlorophenethyl)-3-((4-(methylsulfonyl)phenoxy)methyl)azepane (Compound 1, 200 mg, 0.475 mmol) was separated by preparative chiral SFC (Phenomenex-Cellulose-2, 55% (1:1 MeOH:MeCN) with 0.1% NH₄OH in CO₂). The first eluting enantiomer of the title compound, Compound 2: ¹H NMR (400 MHz, DMSO-d₆): δ 7.82 (d, J=8.8 Hz, 2H), 7.29-7.23 (m, 3H), 7.14-7.11 (m, 3H), 3.90-3.83 (m, 2H), 3.15 (s, 3H), 2.78-2.71 (m, 1H), 2.68-2.57 (m, 7H), 2.01-2.10 (m, 1H), 1.75-1.50 (m, 5H), 1.35-1.32 (m, 1H). MS=422.3 [M+H]⁺. The second eluting enantiomer of the title compound, Compound 3): ¹H NMR (400 MHz, DMSO-d₆): δ 7.82 (d, J=8.8 Hz, 2H), 7.28-7.23 (m, 3H), 7.15-7.11 (m, 3H), 3.90-3.83 (m, 2H), 3.15 (s, 3H), 2.79-2.75 (m, 1H), 2.69-2.53 (m, 7H), 2.01-2.10 (m, 1H), 1.75-1.50 (m, 5H), 1.35-1.32 (m, 1H). MS=422.3 [M+H]⁺.

The following compounds in Table 9 were prepared according to procedures similar to those described for Example 1 using the appropriate starting materials.

TABLE 9

| | | | Exact | | Chiral |
|---|---|---|---|---|---|
| | | | Mass | Chiral | Elution |
| No. | Structure | IUPAC Name | [M + H]⁺ | Column | Order |
| 4 | 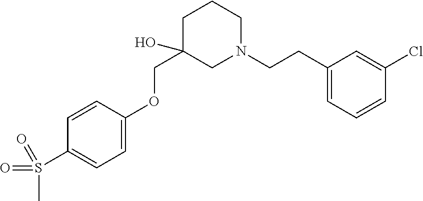 [SMILES: CS(=O)(=O)c1ccc(OCC2(O)CCCN(CCc3cccc(Cl)c3)C2)cc1] | 1-(3- chlorophenethyl)- 3-((4- (methylsulfonyl) phenoxy)methyl) piperidin-3-ol | Calc’d 424.1 Found 424.1 | n/a | n/a |
| 5 | 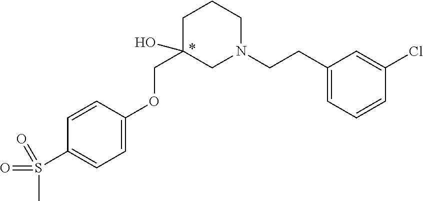 [SMILES: CS(=O)(=O)c1ccc(OCC2(O)CCCN(CCc3cccc(Cl)c3)C2)cc1] | (R) or (S)-1-(3- chlorophenethyl)- 3-((4- (methylsulfonyl) phenoxy)methyl) piperidin-3-ol | Calc’d 424.1 Found 424.2 | Chiralpak AS-3 | 1^{st} |
| 6 | 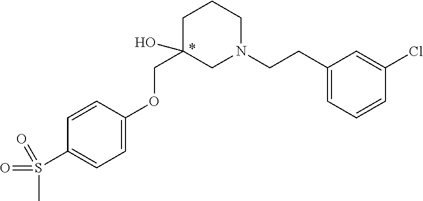 [SMILES: CS(=O)(=O)c1ccc(OCC2(O)CCCN(CCc3cccc(Cl)c3)C2)cc1] | (S) or (R) -1-(3- chlorophenethyl)- 3-((4- (methylsulfonyl) phenoxy)methyl) piperidin-3-ol | Calc’d 424.1 Found 424.2 | Chiralpak AS-3 | 2^{nd} |
| 107 | 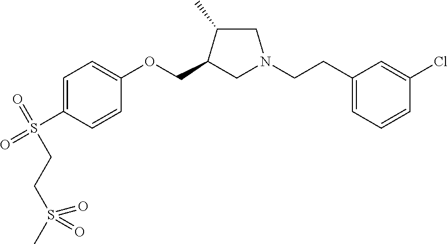 [SMILES: C[C@@H]1CN(CCc2cccc(Cl)c2)C[C@H]1COc1ccc(S(=O)(=O)CCS(C)(=O)=O)cc1] | (3S,4S)-1-[2-(3- chlorophenyl)ethy 1]-3-{[4-(2- methanesulfonyl- ethanesulfonyl) phenoxy]methyl}- 4-methyl- pyrrolidine | Calc’d 500.1 Found 500.1 | n/a | n/a |
| 108 | 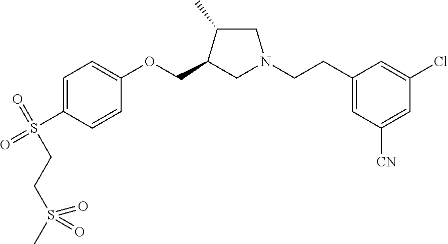 [SMILES: C[C@@H]1CN(CCc2cc(Cl)cc(C#N)c2)C[C@H]1COc1ccc(S(=O)(=O)CCS(C)(=O)=O)cc1] | 3-chloro-5-{2- [(3S,4S)-3-{[4-(2- methanesulfonyl- ethanesulfonyl) phenoxy]methyl}- 4-methyl- pyrrolidin-1-yl] ethyl}benzonitrile | Calc’d 525.1 Found 525.2 | n/a | n/a |
| 109 | 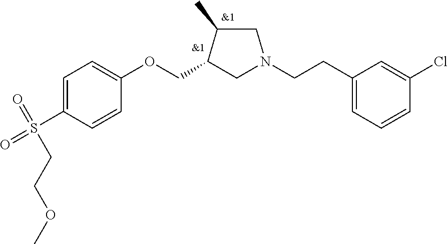 [SMILES: COCCS(=O)(=O)c1ccc(OC[C@H]2CN(CCc3cccc(Cl)c3)C[C@@H]2C)cc1] | rac-trans-1-[2-(3- chlorophenyl) ethyl]-3-{[4-(2- methoxyethane- sulfonyl)phenoxy] methyl}-4- methylpyrrolidine | Calc’d 452.2 Found 452.1 | n/a | n/a |
| 110 | 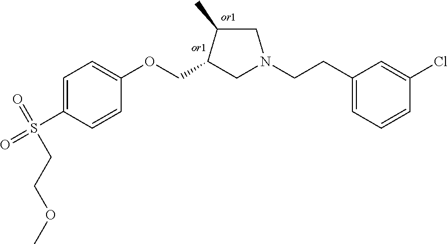 [SMILES: COCCS(=O)(=O)c1ccc(OC[C@H]2CN(CCc3cccc(Cl)c3)C[C@@H]2C)cc1] | (3R,4R or 3S,4S)- 1-[2-(3- chlorophenyl) ethyl]-3-{[4-(2- methoxyethane- sulfonyl)phenoxy] methyl}-4- methylpyrrolidine | Calc’d 452.2 Found 452.1 | Chiralpak AD | 1st |
| 111 | 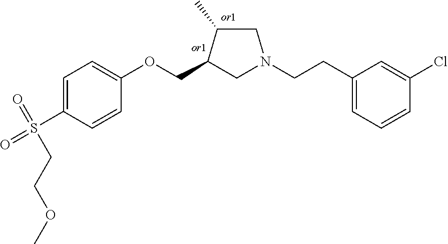 [SMILES: COCCS(=O)(=O)c1ccc(OC[C@@H]2CN(CCc3cccc(Cl)c3)C[C@H]2C)cc1] | (3S,4S or 3R,4R)- 1-[2-(3- chlorophenyl) ethyl]-3-{[4-(2- methoxyethane- sulfonyl)phenoxy] methyl}-4- methylpyrrolidine | Calc’d 452.2 Found 452.1 | Chiralpak AD | 2nd |
| 112 | 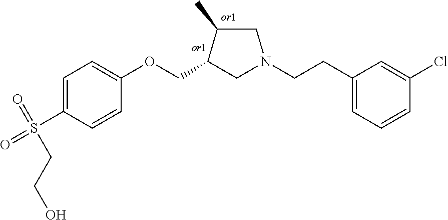 [SMILES: C[C@H]1CN(CCc2cccc(Cl)c2)C[C@@H]1COc1ccc(S(=O)(=O)CCO)cc1] | 2-(4-{[(3R,4R or 3S,4S)-1-[2-(3- chlorophenyl) ethyl]-4- methylpyrrolidin- 3-yl] methoxy}benzene- sulfonyl)ethan-1-ol | Calc’d 438.2 Found 438.1 | n/a | n/a |
| 113 | 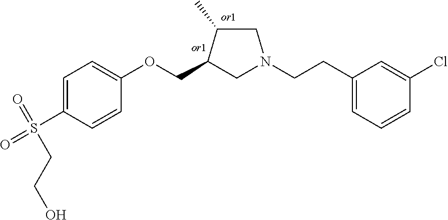 [SMILES: C[C@@H]1CN(CCc2cccc(Cl)c2)C[C@H]1COc1ccc(S(=O)(=O)CCO)cc1] | 2-(4-{[(35,4S or 3R,4R)-1-[2-(3- chlorophenyl) ethyl]-4- methylpyrrolidin- 3-yl] methoxy}benzene- sulfonyl)ethan-1-ol | Calc’d 438.2 Found 438.1 | n/a | n/a |

### Example 2

### N-(4-((1-(3-chlorophenethyl)piperidin-3-yl)methoxy)phenyl)-N-methylmethanesulfonamide (Compound 7)

### (S) or (R)—N-(4-((1-(3-chlorophenethyl)piperidin-3-yl)methoxy)phenyl)-N-methylmethanesulfonamide (Compound 8)

### (R) or (S)—N-(4-((1-(3-chlorophenethyl)piperidin-3-yl)methoxy)phenyl)-N-methylmethanesulfonamide (Compound 9)

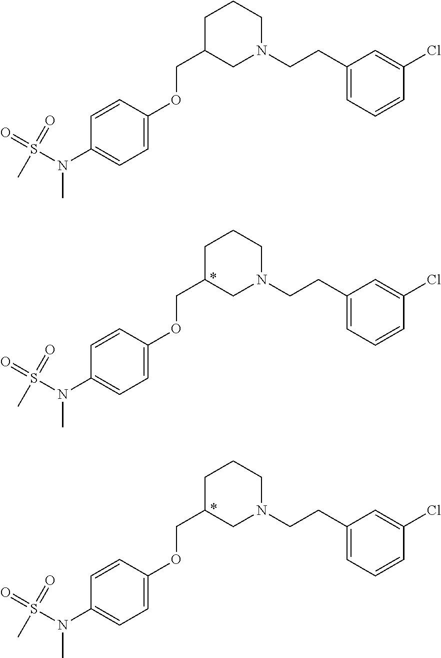 [SMILES: CN(c1ccc(OCC2CCCN(CCc3cccc(Cl)c3)C2)cc1)S(C)(=O)=O ; CN(c1ccc(OCC2CCCN(CCc3cccc(Cl)c3)C2)cc1)S(C)(=O)=O ; CN(c1ccc(OCC2CCCN(CCc3cccc(Cl)c3)C2)cc1)S(C)(=O)=O]

### Step 1: 3-((4-(N-methylmethylsulfonamido)phenoxy)methyl)piperidine-1-carboxylate

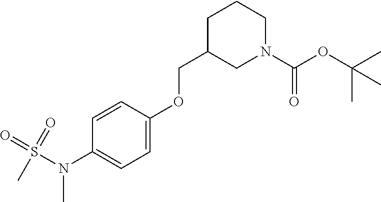 [SMILES: CN(c1ccc(OCC2CCCN(C(=O)OC(C)(C)C)C2)cc1)S(C)(=O)=O]

To a mixture of tert-butyl 3-(bromomethyl)piperidine-1-carboxylate (300 mg, 1.08 mmol) and N-(4-hydroxyphenyl)-N-methylmethanesulfonamide (260 mg, 1.29 mmol) in DMF (3 mL) was added K₂CO₃ (447 mg, 3.24 mmol). The mixture was stirred at 60° C. for 12 h. The reaction mixture was cooled to 0° C. and quenched by the addition of water (15 mL), then extracted with EtOAc (25 mL×2). The combined organic layers were washed with brine (10 mL×2), dried over Na₂SO₄, filtered and concentrated in vacuo. The residue was purified by normal phase silica gel chromatography (Biotage 10 g cartridge, 0-25% EtOAc in petroleum ether) to give tert-butyl 3-((4-(N-methylmethylsulfonamido)phenoxy)methyl)piperidine-1-carboxylate. MS=343.2 [M-C₄H₇+H]⁺.

### Step 2: N-methyl-N-(4-(piperidin-3-ylmethoxy)phenyl)methanesulfonamide

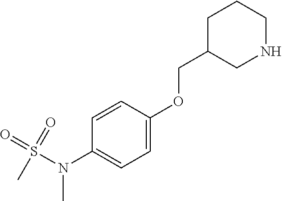 [SMILES: CN(c1ccc(OCC2CCCNC2)cc1)S(C)(=O)=O]

To a solution of tert-butyl 3-((4-(N-methylmethylsulfonamido)phenoxy)methyl) piperidine-1-carboxylate (336 mg, 843.1 mmol) in EtOAc (3 mL) was added HCl/EtOAc (4 M, 3 mL). The mixture was stirred at room temperature for 3 h. The mixture was concentrated in vacuo to give N-methyl-N-(4-(piperidin-3-ylmethoxy)phenyl)methanesulfonamide (HCl salt), which was used in the next step without further purification. MS=299.3 [M+H]⁺.

### Step 3: N-(4-((1-(3-chlorophenethyl)piperidin-3-yl)methoxy)phenyl)-N-methylmethanesulfonamide

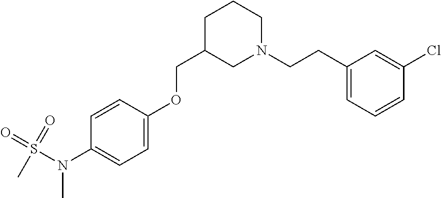 [SMILES: CN(c1ccc(OCC2CCCN(CCc3cccc(Cl)c3)C2)cc1)S(C)(=O)=O]

A mixture of N-methyl-N-(4-(piperidin-3-ylmethoxy)phenyl)methanesulfonamide (181 mg, 0.543 mmol, HCl salt) and 2-(3-chlorophenyl)acetaldehyde (109 mg, 0.706 mmol) in MeOH (2 mL) was stirred at room temperature for 30 min, and then NaBH₃CN (51.2 mg, 0.814 mmol) was added. The resulting mixture was stirred at room temperature for 3 h. The reaction mixture was quenched by the addition of water (0.5 mL) and purified by reverse phase preparative HPLC (Waters Xbridge BEH C18 column, 40-70% MeCN/10 mM NH₄HCO₃ in water) to give N-(4-((1-(3-chlorophenethyl)piperidin-3-yl)methoxy)phenyl)-N-methylmethanesulfonamide (Compound 7).

¹H NMR (400 MHz, DMSO-d₆): δ 7.32-7.19 (m, 6H), 6.94 (d, J=8.8 Hz, 2H), 4.12-4.09 (m, 1H), 3.86-3.81 (m, 2H), 3.18 (m, 3H), 3.17-3.16 (m, 1H), 2.89 (s, 3H), 2.75-2.72 (m, 4H), 2.06-1.95 (m, 3H), 1.76-1.62 (m, 2H), 1.48-1.47 (m, 1H), 1.23-1.10 (m, 1H). MS=437.2 [M+H]⁺.

### Step 4: (S) or (R)—N-(4-((1-(3-chlorophenethyl)piperidin-3-yl)methoxy)phenyl)-N-methylmethanesulfonamide (Compound 8); (R) or (S)—N-(4-((1-(3-chlorophenethyl)piperidin-3-yl)methoxy)phenyl)-N-methylmethanesulfonamide (Compound 9)

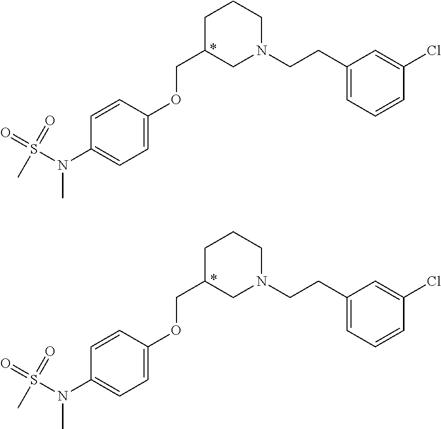 [SMILES: CN(c1ccc(OCC2CCCN(CCc3cccc(Cl)c3)C2)cc1)S(C)(=O)=O ; CN(c1ccc(OCC2CCCN(CCc3cccc(Cl)c3)C2)cc1)S(C)(=O)=O]

N-(4-((1-(3-chlorophenethyl)piperidin-3-yl)methoxy)phenyl)-N-methylmethanesulfonamide (Compound 7, 96 mg, 0.220 mmol) was separated by preparative chiral SFC (Chiralcel OD-3 column, 35% ethanol with 0.1% NH₄OH in CO₂). The first eluting enantiomer of the title compound, Compound 8: ¹H NMR (400 MHz, DMSO-d₆): δ 7.33-7.19 (m, 6H), 6.94 (d, J=9.2 Hz, 2H), 3.86-3.81 (m, 2H), 3.18 (s, 3H), 2.90 (s, 4H), 2.77-2.67 (m, 4H), 2.09-1.92 (m, 4H), 1.75-1.61 (m, 2H), 1.52-1.42 (m, 1H), 1.12-1.09 (m, 1H). MS=437.2 [M+H]⁺. The second eluting enantiomer of the title compound, Compound 9: ¹H NMR (400 MHz, DMSO-d₆): δ 7.33-7.17 (m, 6H), 6.95 (d, J=9.2 Hz, 2H), 3.89-3.84 (m, 2H), 3.18 (s, 3H), 2.90 (s, 4H), 2.78-2.72 (m, 4H), 2.08-1.96 (m, 4H), 1.74-1.65 (m, 2H), 1.50-1.49 (m, 1H), 1.14-1.04 (m, 1H). MS=437.2 [M+H]⁺.

The following compounds in Table 10 were prepared according to procedures similar to those described for Compounds 7-9 using the appropriate starting materials.

TABLE 10

| | | | Exact | | Chiral |
|---|---|---|---|---|---|
| | | | Mass | Chiral | Elution |
| No. | Structure | IUPAC Name | [M + H]⁺ | Column | Order |
| 10 | 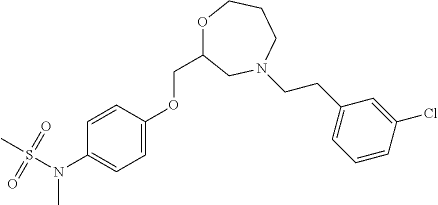 [SMILES: CN(c1ccc(OCC2CN(CCc3cccc(Cl)c3)CCCO2)cc1)S(C)(=O)=O] | N-(4-((4-(3- chlorophenethyl)- 1,4-oxazepan-2- yl)methoxy)phenyl)- N- methylmethane- sulfonamide | Calc'd 453.2 Found 453.2 | n/a | n/a |
| 11 | 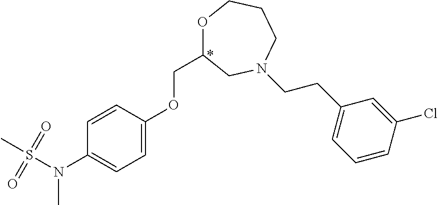 [SMILES: CN(c1ccc(OCC2CN(CCc3cccc(Cl)c3)CCCO2)cc1)S(C)(=O)=O] | (R) or (S)-N-(4- ((4-(3- chlorophenethyl)- 1,4-oxazepan-2- yl)methoxy)phenyl)- N- methylmethane- sulfonamide | Calc'd 453.2 Found 453.2 | Chiralpak AD-3 | 1st |
| 12 | 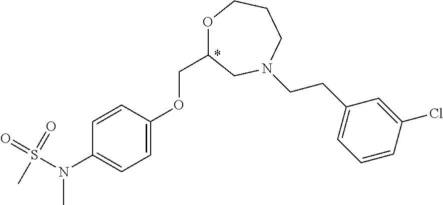 [SMILES: CN(c1ccc(OCC2CN(CCc3cccc(Cl)c3)CCCO2)cc1)S(C)(=O)=O] | (S) or (R)- N-(4- ((4-(3- chlorophenethyl)- 1,4-oxazepan-2- yl)methoxy)phenyl)- N- methylmethane- sulfonamide | Calc'd 453.2 Found 453.2 | Chiralpak AD-3 | 2nd |

### Example 3

### (S)-1-(3-chlorophenethyl)-3-((4-(methylsulfonyl)phenoxy)methyl)piperazine (Compound 13)

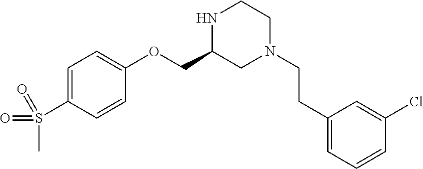 [SMILES: CS(=O)(=O)c1ccc(OC[C@@H]2CN(CCc3cccc(Cl)c3)CCN2)cc1]

### Step 1: (S)-1-benzyl 4-tert-butyl 2-(hydroxymethyl)piperazine-1,4-dicarboxylate

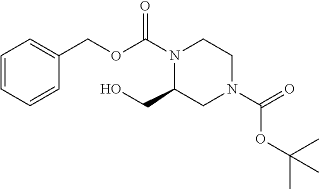 [SMILES: CC(C)(C)OC(=O)N1CCN(C(=O)OCc2ccccc2)[C@H](CO)C1]

To a mixture of (S)-tert-butyl 3-(hydroxymethyl)piperazine-1-carboxylate (1.00 g, 4.62 mmol) and NaHCO₃ (1.17 g, 13.9 mmol) in THF (5 mL) and water (5 mL) at 0° C. was added benzyl chloroformate (986 μL, 6.94 mmol). The mixture was stirred at room temperature for 4 h, then quenched by the addition of water (20 mL) and extracted with EtOAc (20 mL×3). The combined organic layers were washed with brine (60 mL), dried over Na₂SO₄, filtered and concentrated in vacuo. The crude residue was purified by normal phase silica gel chromatography (Biotage 20 g cartridge, 7-50% EtOAc in petroleum ether) to give (S)-1-benzyl 4-tert-butyl 2-(hydroxymethyl)piperazine-1,4-dicarboxylate. MS=373.2 [M+Na]⁺.

### Step 2: (S)-1-benzyl 4-tert-butyl 2-((4-(methylsulfonyl)phenoxy)methyl)piperazine-1,4-dicarboxylate

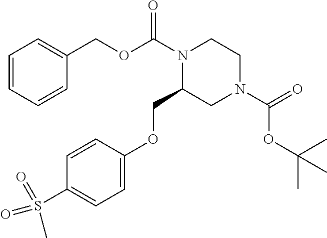 [SMILES: CC(C)(C)OC(=O)N1CCN(C(=O)OCc2ccccc2)[C@H](COc2ccc(S(C)(=O)=O)cc2)C1]

To a mixture of (S)-1-benzyl 4-tert-butyl 2-(hydroxymethyl)piperazine-1,4-dicarboxylate (200 mg, 0.571 mmol), 4-methylsulfonylphenol (98.0 mg, 0.571 mmol) and PPh₃ (225 mg, 0.856 mmol) in THF (4 mL) at 0° C. was added DIAD (222 μL, 1.14 mmol) dropwise. The mixture was stirred at 0° C. for 2 h, and then quenched by the addition of water (20 mL). The mixture was extracted with EtOAc (20 mL×3). The combined organic layers were washed with brine (60 mL), dried over Na₂SO₄, filtered and concentrated in vacuo. The crude residue was purified by normal phase silica gel chromatography (Biotage 4 g cartridge, 0-40% EtOAc in petroleum ether) to give (S)-1-benzyl 4-tert-butyl 2-((4-(methylsulfonyl)phenoxy)methyl)piperazine-1,4-dicarboxylate. MS=527.3 [M+Na]⁺.

### Step 3: (S)-benzyl 2-((4-(methylsulfonyl)phenoxy)methyl)piperazine-1-carboxylate

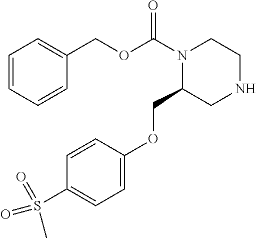 [SMILES: CS(=O)(=O)c1ccc(OC[C@@H]2CNCCN2C(=O)OCc2ccccc2)cc1]

To a solution of (S)-1-benzyl 4-tert-butyl 2-((4-(methylsulfonyl)phenoxy)methyl)piperazine-1,4-dicarboxylate (600 mg, 1.12 mmol) in EtOAc (5 mL) was added HCl/EtOAc (4 M, 12 mL). The mixture was stirred at room temperature for 2 h, then concentrated in vacuo. The residue was diluted with water (20 mL) and washed with EtOAc (20 mL). The aqueous layer pH was adjusted to pH=8-9 by addition of saturated aqueous NaHCO₃ and extracted with EtOAc (20 mL×3). The combined organic layers were washed with brine (60 mL), dried over Na₂SO₄, filtered and concentrated in vacuo to give (S)-benzyl 2-((4-(methylsulfonyl)phenoxy)methyl)piperazine-1-carboxylate, which was used in the next step without further purification. MS=405.2 [M+H]⁺.

### Step 4: (S)-benzyl 4-(3-chlorophenethyl)-2-((4-(methylsulfonyl)phenoxy)methyl) piperazine-1-carboxylate

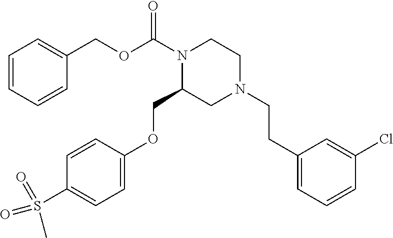 [SMILES: CS(=O)(=O)c1ccc(OC[C@@H]2CN(CCc3cccc(Cl)c3)CCN2C(=O)OCc2ccccc2)cc1]

To a solution of (S)-benzyl 2-((4-(methylsulfonyl)phenoxy)methyl)piperazine-1-carboxylate (200 mg, 0.494 mmol) and 2-(3-chlorophenyl)acetaldehyde (76.0 mg, 0.494 mmol) in MeOH (2 mL) at 0° C. was added NaBH₃CN (47.0 mg, 0.742 mmol) slowly. The mixture was stirred at room temperature for 1 h, then was quenched with water (0.05 mL) and concentrated in vacuo. The crude residue was purified by normal phase silica gel chromatography (Biotage 12 g cartridge, 10-50% EtOAc in petroleum ether) to give (S)-benzyl 4-(3-chlorophenethyl)-2-((4-(methylsulfonyl)phenoxy)methyl)piperazine-1-carboxylate. MS=543.2 [M+H]⁺.

### Step 5: (S)-1-(3-chlorophenethyl)-3-((4-(methylsulfonyl)phenoxy)methyl)piperazine

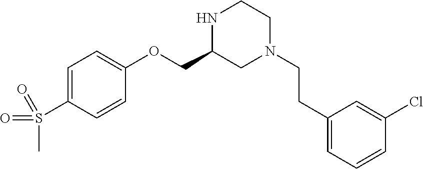 [SMILES: CS(=O)(=O)c1ccc(OC[C@@H]2CN(CCc3cccc(Cl)c3)CCN2)cc1]

To a solution of (S)-benzyl 4-(3-chlorophenethyl)-2-((4-(methylsulfonyl)phenoxy)methyl)piperazine-1-carboxylate (120 mg, 0.221 mmol) and NaI (331 mg, 2.21 mmol) in MeCN (3 mL) at 0° C. was added TMSCl (280 μL, 2.21 mmol) dropwise. The mixture was stirred at room temperature for 16 h, then was quenched with water (0.2 mL) and purified by preparative HPLC (Waters Xbridge BEH C18 column, 30-55% MeCN/10 mM NH₄HCO₃ in water) to give (S)-1-(3-chlorophenethyl)-3-((4-(methylsulfonyl)phenoxy)methyl)piperazine (Compound 13). ¹H NMR (400 MHz, DMSO-d₆, 24/25 H): δ 7.83 (d, J=8.8 Hz, 2H), 7.32-7.15 (m, 6H), 4.01-3.94 (m, 2H), 3.32 (s, 3H), 3.15-3.14 (s, 1H), 2.88-2.85 (m, 2H), 2.76-2.66 (m, 4H), 2.47-2.44 (m, 2H), 2.07-2.00 (m, 1H), 1.89 (app t, J=10.0 Hz, 1H). MS=409.3 [M+H]⁺.

The following compound in Table 11 was prepared according to procedures similar to those described for Compound 13 using the appropriate starting materials.

TABLE 11

| | | | Exact Mass |
|---|---|---|---|
| No. | Structure | IUPAC Name | [M + H]⁺ |
| 14 | 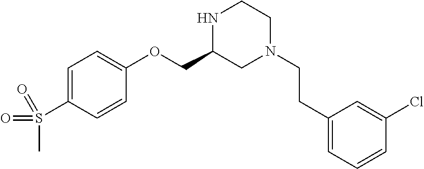 [SMILES: CS(=O)(=O)c1ccc(OC[C@@H]2CN(CCc3cccc(Cl)c3)CCN2)cc1] | (R)-1-(3-chlorophenethyl)-3- ((4- (methylsulfonyl)phenoxy) methyl)piperazine | Calc'd 409.1 Found 409.1 |

### Example 4

### (R)-1-(3-chlorophenethyl)-3-((4-(methylsulfonyl)phenoxy)methyl)piperidine (Compound 15)

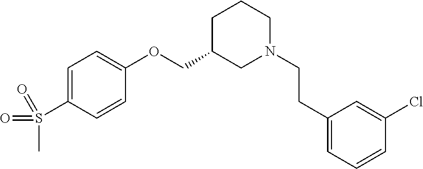 [SMILES: CS(=O)(=O)c1ccc(OC[C@@H]2CCCN(CCc3cccc(Cl)c3)C2)cc1]

To a solution (R)-3-((4-(methylsulfonyl)phenoxy)methyl)piperidine (Intermediate G, 56.9. mg, 0.186 mmol, HCl salt) in DCM (5 mL) was added TEA (155 μL, 1.11 mmol) and 2-(3-chlorophenyl)acetaldehyde (28.70 mg, 0.186 mmol). The mixture was stirred at room temperature for 30 min, then NaBH(OAc)₃ (157 mg, 0.743 mmol) was added. After stirring for 12 h, the reaction mixture was quenched with water (5 mL), and then extracted with EtOAc (5 mL×3). The combined organic layers were washed with brine (5 mL×3), dried over Na₂SO₄, filtered and concentrated in vacuo. The crude residue was purified by reverse phase preparative HPLC (Waters Xbridge BEH C18, 50-80% MeCN/10 mM NH₄HCO₃ in water) to give (R)-1-(3-chlorophenethyl)-3-((4-(methylsulfonyl)phenoxy)methyl)piperidine (Compound 15). ¹H NMR (400 MHz, DMSO-d₆): δ 7.82 (d, J=6.8 Hz, 2H), 7.30-7.13 (m, 6H), 4.00-3.93 (m, 2H), 3.15 (s, 3H), 2.90 (d, J=7.6 Hz, 1H), 2.77-2.71 (m, 3H), 2.53-2.52 (m, 2H), 2.05-1.97 (m, 3H), 1.66-1.64 (m, 1H), 1.64-1.63 (m, 1H), 1.49-1.41 (m, 1H), 1.17-1.12 (m, 1H). MS=408.2 [M+H]⁺.

The following compound in Table 12 was prepared according to procedures similar to those described for Compound 15 using the appropriate starting materials.

TABLE 12

| | | | | Exact |
|---|---|---|---|---|
| | Inter- | | | Mass |
| No. | mediate | Structure | IUPAC Name | [M + H]⁺ |
| 16 | H | 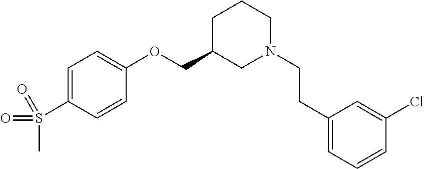 [SMILES: CS(=O)(=O)c1ccc(OC[C@H]2CCCN(CCc3cccc(Cl)c3)C2)cc1] | (S)-1-(3- chlorophenethyl)-3- ((4- (methylsulfonyl) phenoxy)methyl) piperidine | Calc'd 408.1 Found 408.1 |

### Example 5

### 1-(3-chlorophenyl)-2-((S)-3-((4-(methylsulfonyl)phenoxy)methyl)piperidin-1-yl)ethan-1-ol (Compound 17)

### (R) or (S)-1-(3-chlorophenyl)-2-((S)-3-((4-(methylsulfonyl)phenoxy)methyl)piperidin-1-yl)ethan-1-ol (Compound 18)

### (S) or (R)-1-(3-chlorophenyl)-2-((S)-3-((4-(methylsulfonyl)phenoxy)methyl)piperidin-1-yl)ethan-1-ol (Compound 19)

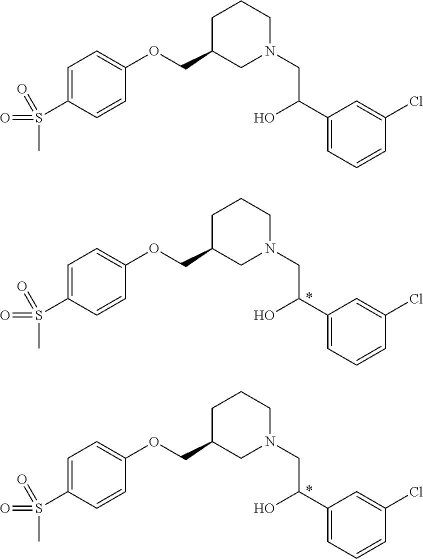 [SMILES: CS(=O)(=O)c1ccc(OC[C@H]2CCCN(CC(O)c3cccc(Cl)c3)C2)cc1 ; CS(=O)(=O)c1ccc(OC[C@H]2CCCN(CC(O)c3cccc(Cl)c3)C2)cc1 ; CS(=O)(=O)c1ccc(OC[C@H]2CCCN(CC(O)c3cccc(Cl)c3)C2)cc1]

### Step 1: 1-(3-chlorophenyl)-2-((S)-3-((4-(methylsulfonyl)phenoxy)methyl)piperidin-1-yl)ethanol

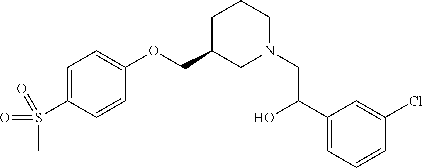 [SMILES: CS(=O)(=O)c1ccc(OC[C@H]2CCCN(CC(O)c3cccc(Cl)c3)C2)cc1]

To a solution of (S)-3-((4-(methylsulfonyl)phenoxy)methyl)piperidine (Intermediate H, 500 mg, 1.63 mmol, HCl salt) and 2-(3-chlorophenyl)oxirane (379 mg, 2.45 mmol) in EtOH (10 mL) was added TEA (455 μL, 3.27 mmol). The mixture was stirred at 80° C. for 8 h, then cooled to room temperature and quenched by the addition of water (1 mL), filtered, and concentrated in vacuo. The crude residue was purified by reverse phase preparative HPLC (Phenomenex Gemini-NX, 25-55% MeCN/10 mM NH₄HCO₃ in water) to give 1-(3-chlorophenyl)-2-((S)-3-((4-(methylsulfonyl)phenoxy)methyl)piperidin-1-yl)ethanol (Compound 17). ¹H NMR (400 MHz, DMSO-d₆): δ 7.82 (d, J=8.8 Hz, 2H), 7.38 (s, 1H), 7.30-7.27 (m, 3H), 7.16-7.13 (m, 2H), 5.14 (s, 1H), 4.70-4.69 (m, 1H), 3.99-3.92 (m, 2H), 3.15 (s, 3H), 2.95-2.85 (m, 1H), 2.75-2.74 (m, 1H), 2.46-2.45 (m, 1H), 2.41-2.35 (m, 1H), 2.17-1.98 (m, 3H), 1.72-1.60 (m, 2H), 1.50-1.45 (m, 1H), 1.15-1.12 (m, 1H). MS=424.1 [M+H]⁺.

### Step 2: (R) or (S)-1-(3-chlorophenyl)-2-((S)-3-((4-(methylsulfonyl)phenoxy)methyl)piperidin-1-yl)ethan-1-ol (Compound 18); (S) or (R)-1-(3-chlorophenyl)-2-((S)-3-((4-(methylsulfonyl)phenoxy)methyl)piperidin-1-yl)ethan-1-ol (Compound 19)

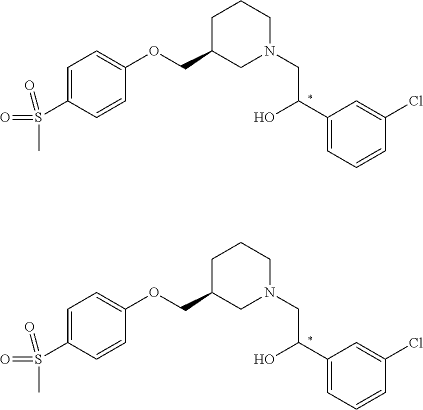 [SMILES: CS(=O)(=O)c1ccc(OC[C@H]2CCCN(CC(O)c3cccc(Cl)c3)C2)cc1 ; CS(=O)(=O)c1ccc(OC[C@H]2CCCN(CC(O)c3cccc(Cl)c3)C2)cc1]

1-(3-chlorophenyl)-2-[(3S)-3-[(4-methylsulfonylphenoxy)methyl]-1-piperidyl]ethanol (Compound 17, 400 mg, 0.944 mmol) was separated by preparative chiral SFC (DAICEL CHIRALPAK AD-3, 55% ethanol with 0.1% NH₄OH in CO₂). The first eluting enantiomer of the title compound, Compound 18: ¹H NMR (400 MHz, DMSO-d₆): δ 7.83 (d, J=8.8 Hz, 2H), 7.38 (s, 1H), 7.30-7.25 (m, 3H), 7.16-7.13 (m, 2H), 5.14 (d, J=3.6 Hz, 1H), 4.71-4.70 (m, 1H), 3.95-3.94 (m, 2H), 3.15 (s, 3H), 2.87-2.74 (m, 2H), 2.46-2.41 (m, 1H), 2.40-2.38 (m, 1H), 2.15-1.98 (m, 3H), 1.69-1.60 (m, 2H), 1.50-1.49 (m, 1H), 1.17-1.12 (m, 1H). MS=424.1 [M+H]⁺. The second eluting enantiomer of the title compound, Compound 19: ¹H NMR (400 MHz, DMSO-d₆): δ 7.83 (d, J=8.8 Hz, 2H), 7.39-7.13 (m, 6H), 5.15 (s, 1H), 4.71 (s, 1H), 3.99-3.90 (m, 2H), 3.15 (s, 3H), 2.96-2.94 (m, 1H), 2.77-2.75 (m, 1H), 2.46-2.41 (m, 1H), 2.40-2.38 (m, 1H), 2.14-2.04 (m, 3H), 1.73-1.61 (m, 2H), 1.49-1.46 (m, 1H), 1.14-1.12 (m, 1H). MS=424.1 [M+H]⁺.

The following compounds in Table 13 were prepared according to procedures similar to those described for Compound 17 using the appropriate starting materials.

TABLE 13

| | | | Exact | | Chiral |
|---|---|---|---|---|---|
| | | | Mass | Chiral | Elution |
| No. | Structure | IUPAC Name | [M + H]⁺ | Column | Order |
| 20 | 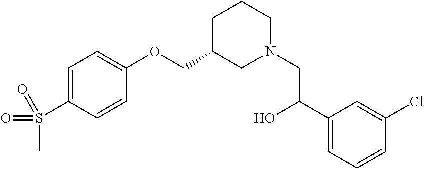 [SMILES: CS(=O)(=O)c1ccc(OC[C@@H]2CCCN(CC(O)c3cccc(Cl)c3)C2)cc1] | 1-(3- chlorophenyl)-2- ((R)-3-((4- (methylsulfonyl) phenoxy)methyl) piperidin-1- yl)ethanol | Calc'd 424.1 Found 424.3 | n/a | n/a |
| 21 | 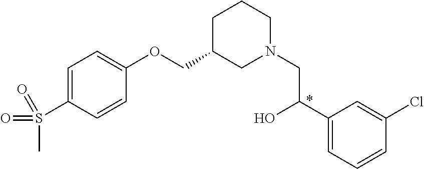 [SMILES: CS(=O)(=O)c1ccc(OC[C@@H]2CCCN(CC(O)c3cccc(Cl)c3)C2)cc1] | (R) or (S)-1-(3- chlorophenyl)-2- ((R)-3-((4- (methylsulfonyl) phenoxy)methyl) piperidin-1- yl)ethanol | Calc'd 424.1 Found 424.2 | Chiralpak AD-3 | 1st |
| 22 | 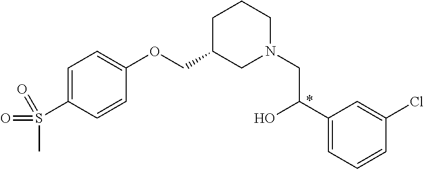 [SMILES: CS(=O)(=O)c1ccc(OC[C@@H]2CCCN(CC(O)c3cccc(Cl)c3)C2)cc1] | (S) or (R)-1-(3- chlorophenyl)-2- ((R)-3-((4- (methylsulfonyl) phenoxy)methyl) piperidin-1- yl)ethanol | Calc'd 424.1 Found 424.2 | Chiralpak AD-3 | 2nd |
| 114 | 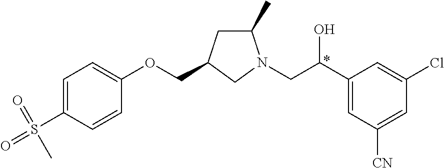 [SMILES: C[C@@H]1C[C@H](COc2ccc(S(C)(=O)=O)cc2)CN1CC(O)c1cc(Cl)cc(C#N)c1] | 3-chloro-5-[(1S or 1R)-1- hydroxy-2- [(2R,4S)-4-[(4- methanesulfonyl phenoxy)methyl]- 2- methylpyrrolidin- 1- yl]ethyl] benzonitrile | Calc'd 449.1 Found 449.1 | prep- HPLC Phenomenex Gemini- NX 80 | 1st |
| 115 | 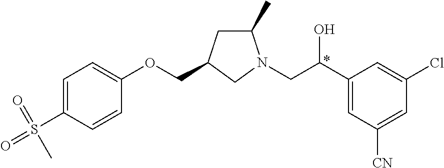 [SMILES: C[C@@H]1C[C@H](COc2ccc(S(C)(=O)=O)cc2)CN1CC(O)c1cc(Cl)cc(C#N)c1] | 3-chloro-5-[(1R or 1S)-1- hydroxy-2- [(2R,4S)-4-[(4- methanesulfonyl phenoxy)methyl]- 2- methylpyrrolidin- 1- yl]ethyl] benzonitrile | Calc'd 449.1 Found 449.1 | prep- HPLC Phenomenex Gemini- NX 80 | 2nd |
| 116 | 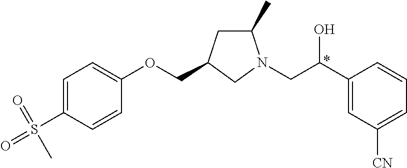 [SMILES: C[C@@H]1C[C@H](COc2ccc(S(C)(=O)=O)cc2)CN1CC(O)c1cccc(C#N)c1] | 3-[(1R or 1S)-1- hydroxy-2- [(2R,4S)-4-[(4- methanesulfonyl phenoxy)methyl]- 2- methylpyrrolidin- 1- yl]ethyl] benzonitrile | Calc'd 415.2 Found 415.2 | prep- HPLC Phenomenex Gemini- NX | 2nd |
| 117 | 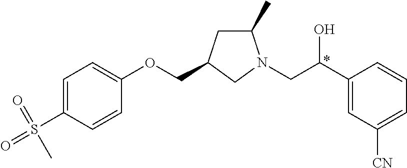 [SMILES: C[C@@H]1C[C@H](COc2ccc(S(C)(=O)=O)cc2)CN1CC(O)c1cccc(C#N)c1] | 3-[(1S or 1R)-1- hydroxy-2- [(2R,4S)-4-[(4- methanesulfonyl phenoxy)methyl]- 2- methylpyrrolidin- 1- yl]ethyl] benzonitrile | Calc'd 415.2 Found 415.2 | prep- HPLC Phenomenex Gemini- NX | 1st |
| 214 | 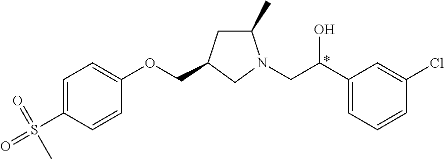 [SMILES: C[C@@H]1C[C@H](COc2ccc(S(C)(=O)=O)cc2)CN1CC(O)c1cccc(Cl)c1] | (1R) or (1S)-1- (3- chlorophenyl)-2- [(2R,4S)-4-[(4- methanesulfonyl phenoxy)methyl]- 2- methylpyrrolidin- 1-yl]ethan-1-ol | Calc'd 424.1 Found 424.2 | prep- HPLC Phenomenex Gemini- NX | 1st |
| 215 | 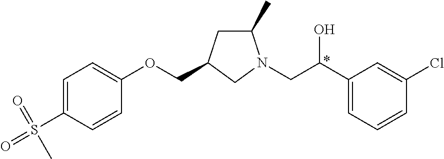 [SMILES: C[C@@H]1C[C@H](COc2ccc(S(C)(=O)=O)cc2)CN1CC(O)c1cccc(Cl)c1] | (1S) or (1R)-1- (3- chlorophenyl)-2- [(2R,4S)-4-[(4- methanesulfonyl phenoxy)methyl]- 2- methylpyrrolidin- 1-yl]ethan-1-ol | Calc'd 424.1 Found 424.2 | prep- HPLC Phenomenex Gemini- NX | 2nd |
| 118 | 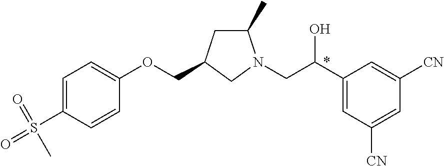 [SMILES: C[C@@H]1C[C@H](COc2ccc(S(C)(=O)=O)cc2)CN1CC(O)c1cc(C#N)cc(C#N)c1] | 5-[(1R or 1S)-1- hydroxy-2- [(2R,4S)-4-[(4- methanesulfonyl phenoxy)methyl]- 2- methylpyrrolidin- 1- ylethyl]benzene- 1,3- dicarbonitrile | Calc'd 440.2 Found 440.2 | prep- HPLC Phenomenex C18 | 1st |
| 119 | 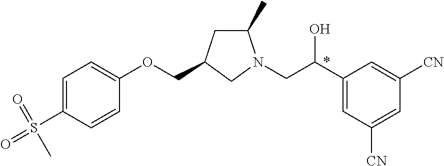 [SMILES: C[C@@H]1C[C@H](COc2ccc(S(C)(=O)=O)cc2)CN1CC(O)c1cc(C#N)cc(C#N)c1] | 5-[(1S or 1R)-1- hydroxy-2- [(2R,4S)-4-[(4- methanesulfonyl phenoxy)methyl]- 2- methylpyrrolidin- 1- yl]ethyl]benzene- 1,3- dicarbonitrile | Calc'd 440.2 Found 440.0 | prep- HPLC Phenomenex C18 | 2nd |
| 120 | 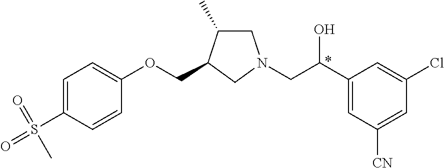 [SMILES: C[C@@H]1CN(CC(O)c2cc(Cl)cc(C#N)c2)C[C@H]1COc1ccc(S(C)(=O)=O)cc1] | 3-chloro-5-{1- hydroxy-2- [(35,4S)-3-[(4- methanesulfonyl phenoxy)methyl]- 4- methylpyrrolidin- 1- ylethyl} benzonitrile | Calc'd 449.1 Found 449.0 | n/a | n/a |
| 121 | 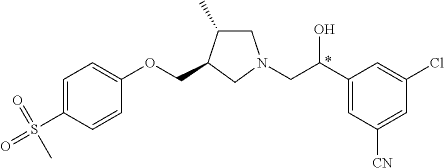 [SMILES: C[C@@H]1CN(CC(O)c2cc(Cl)cc(C#N)c2)C[C@H]1COc1ccc(S(C)(=O)=O)cc1] | 3-chloro-5-[(1S or 1R)-1- hydroxy-2- [(3S,4S)-3-[(4- methanesulfonyl phenoxy)methyl]- 4- methylpyrrolidin- 1- ylethyl] benzonitrile | Calc'd 449.1 Found 449.0 | Phenomenex- Cellulose- 2 | 1st |
| 122 | 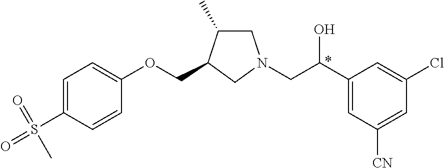 [SMILES: C[C@@H]1CN(CC(O)c2cc(Cl)cc(C#N)c2)C[C@H]1COc1ccc(S(C)(=O)=O)cc1] | 3-chloro-5-[(1R or 1S)-1- hydroxy-2- [(3S,4S)-3-[(4- methanesulfonyl phenoxy)methyl]- 4- methylpyrrolidin- 1- yl]ethyl] benzonitrile | Calc'd 449.1 Found 449.0 | Phenomenex- Cellulose- 2 | 2nd |
| 123 | 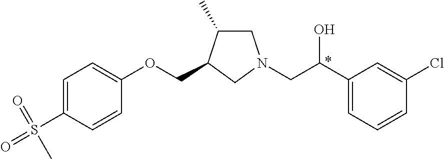 [SMILES: C[C@@H]1CN(CC(O)c2cccc(Cl)c2)C[C@H]1COc1ccc(S(C)(=O)=O)cc1] | 1-(3- chlorophenyl)-2- ((3S,4S)-3- methyl-4-((4- (methylsulfonyl) phenoxy)methyl) pyrrolidin-1- yl)ethan-1-ol | Calc'd 424.1 Found 424.1 | n/a | n/a |
| 124 | 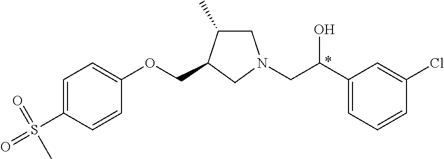 [SMILES: C[C@@H]1CN(CC(O)c2cccc(Cl)c2)C[C@H]1COc1ccc(S(C)(=O)=O)cc1] | (1R or 1S)-1-(3- chlorophenyl)-2- [(3S,4S)-3-[(4- methanesulfonyl phenoxy)methyl]- 4- methylpyrrolidin- 1-yl]ethan-1-ol | Calc'd 424.1 Found 424.0 | Phenomenex- Cellulose- 2 | 1st |
| 125 | 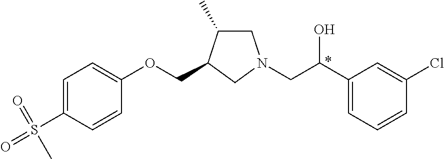 [SMILES: C[C@@H]1CN(CC(O)c2cccc(Cl)c2)C[C@H]1COc1ccc(S(C)(=O)=O)cc1] | (1S or 1R)-1-(3- chlorophenyl)-2- [(3S,4S)-3-[(4- methanesulfonyl phenoxy)methyl]- 4- methylpyrrolidin- 1-yl]ethan-1-ol | Calc'd 424.1 Found 424.0 | Phenomenex- Cellulose- 2 | 2nd |
| 126 | 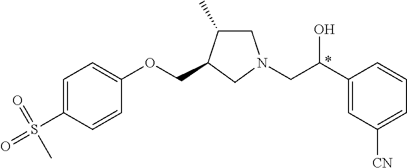 [SMILES: C[C@@H]1CN(CC(O)c2cccc(C#N)c2)C[C@H]1COc1ccc(S(C)(=O)=O)cc1] | 3-[1-hydroxy-2- [(3S,4S)-3-[(4- methanesulfonyl phenoxy)methyl]- 4- methylpyrrolidin- 1- ylethyl] benzonitrile | Calc'd 415.2 Found 415.0 | n/a | n/a |
| 127 | 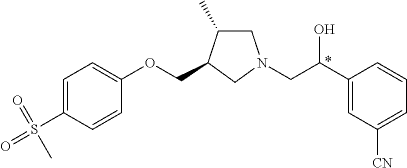 [SMILES: C[C@@H]1CN(CC(O)c2cccc(C#N)c2)C[C@H]1COc1ccc(S(C)(=O)=O)cc1] | 3-[(1S or 1R)-1- hydroxy-2- [(3S,4S)-3-[(4- methanesulfonyl phenoxy)methyl]- 4- methylpyrrolidin- 1- yl]ethyl] benzonitrile | Calc'd 415.2 Found 415.0 | Chiralpk AD | 1st |
| 128 | 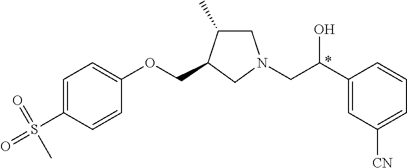 [SMILES: C[C@@H]1CN(CC(O)c2cccc(C#N)c2)C[C@H]1COc1ccc(S(C)(=O)=O)cc1] | 3-[(1R or 1S)-1- hydroxy-2- [(35,4S)-3-[(4- methanesulfonyl phenoxy)methyl]- 4- methylpyrrolidin- 1- ylethyl] benzonitrile | Calc'd 415.2 Found 415.0 | Chiralpk AD | 2nd |
| 129 | 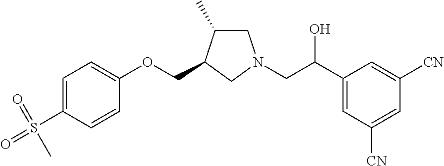 [SMILES: C[C@@H]1CN(CC(O)c2cc(C#N)cc(C#N)c2)C[C@H]1COc1ccc(S(C)(=O)=O)cc1] | 5-{ 1-hydroxy-2- [(3S,4S)-3-[(4- methanesulfonyl phenoxy)methyl]- 4- methylpyrrolidin- 1- yl]ethyl}benzene- 1,3- dicarbonitrile | Calc'd 440.2 Found 440.2 | n/a | n/a |
| 130 | 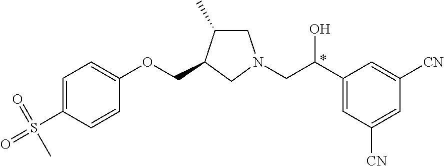 [SMILES: C[C@@H]1CN(CC(O)c2cc(C#N)cc(C#N)c2)C[C@H]1COc1ccc(S(C)(=O)=O)cc1] | 5-[(1R or 1S)-1- hydroxy-2- [(3S,4S)-3-[(4- methanesulfonyl phenoxy)methyl]- 4- methylpyrrolidin- 1- yl]ethyl]benzene- 1,3- dicarbonitrile | Calc'd 440.2 Found 440.2 | Chiralpk AD | 1st |
| 131 | 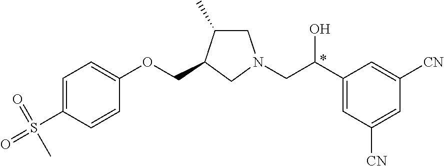 [SMILES: C[C@@H]1CN(CC(O)c2cc(C#N)cc(C#N)c2)C[C@H]1COc1ccc(S(C)(=O)=O)cc1] | 5-[(1S or 1R)-1- hydroxy-2- [(35,4S)-3-[(4- methanesulfonyl phenoxy)methyl]- 4- methylpyrrolidin- 1- yl]ethyl]benzene- 1,3- dicarbonitrile | Calc'd 440.2 Found 440.2 | Chiralpk AD | 2nd |

### Example 6

### (S)-1-((R)-2-(3-chlorophenyl)-2-methoxyethyl)-3-((4-(methylsulfonyl)phenoxy)methyl)piperidine or (S)-1-((S)-2-(3-chlorophenyl)-2-methoxyethyl)-3-((4-(methylsulfonyl)phenoxy)methyl)piperidine (Compound 23)

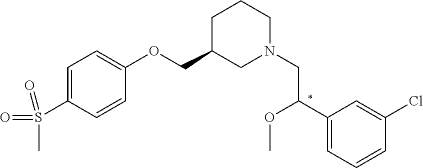 [SMILES: COC(CN1CCC[C@H](COc2ccc(S(C)(=O)=O)cc2)C1)c1cccc(Cl)c1]

A solution of (R) or (S)-1-(3-chlorophenyl)-2-((S)-3-((4-(methylsulfonyl)phenoxy)methyl)piperidin-1-yl)ethan-1-ol (Compound 18, 50.0 mg, 0.118 mmol) in THF (2 mL) at 0° C. was added NaH (5.66 mg, 0.142 mmol, 60% dispersion in mineral oil). After stirring at 0° C. for 1 h, MeI (8.08 μL, 0.130 mmol) was added and the mixture was allowed to warm to room temperature. After stirring for 1 h, the reaction mixture was cooled to 0° C. and quenched by the addition of water (5 mL), and extracted with EtOAc (5 mL×3). The combined organic layers were washed with brine (5 mL), dried over Na₂SO₄, filtered and concentrated in vacuo. The crude residue was purified by reverse phase preparative HPLC (Waters Xbridge BEH C18 column, 35-70% MeCN/10 mM NH₄HCO₃ in water) to give (S)-1-((R)-2-(3-chlorophenyl)-2-methoxyethyl)-3-((4-(methylsulfonyl)phenoxy)methyl)piperidine or (S)-1-((S)-2-(3-chlorophenyl)-2-methoxyethyl)-3-((4-(methylsulfonyl)phenoxy)methyl)piperidine (Compound 23). ¹H NMR (400 MHz, DMSO-d₆): δ 7.83 (d, J=8.8 Hz, 2H), 7.37-7.34 (m, 3H), 7.27-7.25 (m, 1H), 7.15-7.13 (m, 2H), 4.38 (t, J=6.0 Hz, 1H), 3.93-3.89 (m, 2H), 3.15 (s, 3H), 3.13 (s, 3H), 2.80-2.77 (m, 2H), 2.43-2.40 (m, 2H), 2.15-2.08 (m, 2H), 1.96-1.95 (m, 1H), 1.70-1.59 (m, 2H), 1.45-1.43 (m, 1H), 1.14-1.11 (m, 1H). MS=438.1 [M+H]⁺.

The following compounds in Table 14 were prepared according to procedures similar to those described for Compound 23 using the appropriate starting materials.

TABLE 14

| | | | | Exact |
|---|---|---|---|---|
| | Starting | | | Mass |
| No. | Material | Structure | IUPAC Name | [M + H]⁺ |
| 24 | Compound 19 | 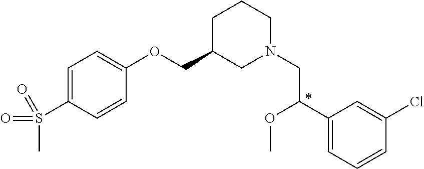 [SMILES: COC(CN1CCC[C@H](COc2ccc(S(C)(=O)=O)cc2)C1)c1cccc(Cl)c1] | (S)-1-((S)-2-(3- chlorophenyl)-2- methoxyethyl)-3-((4- (methylsulfonyl)phenoxy) methyl)piperidine or (S)-1-((R)-2-(3- chlorophenyl)-2- methoxyethyl)-3-((4- (methylsulfonyl)phenoxy) methyl)piperidine | Calc'd 438.1 Found 438.1 |
| 25 | Compound 21 | 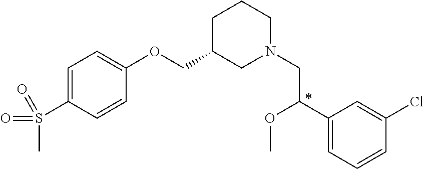 [SMILES: COC(CN1CCC[C@@H](COc2ccc(S(C)(=O)=O)cc2)C1)c1cccc(Cl)c1] | (R)-1-((R)-2-(3- chlorophenyl)-2- methoxyethyl)-3-((4- (methylsulfonyl)phenoxy) methyl)piperidine or (R)-1-((S)-2-(3- chlorophenyl)-2- methoxyethyl)-3-((4- (methylsulfonyl)phenoxy) methyl)piperidine | Calc'd 438.1 Found 438.3 |
| 26 | Compound 22 | 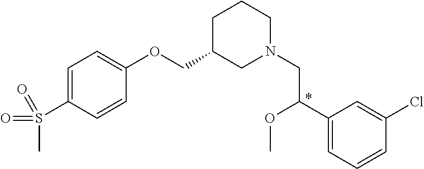 [SMILES: COC(CN1CCC[C@@H](COc2ccc(S(C)(=O)=O)cc2)C1)c1cccc(Cl)c1] | (R)-1-((S)-2-(3- chlorophenyl)-2- methoxyethyl)-3-((4- (methylsulfonyl)phenoxy) methyl)piperidine or (R)-1-((R)-2-(3- chlorophenyl)-2- methoxyethyl)-3-((4- (methylsulfonyl)phenoxy) methyl)piperidine | Calc'd 438.1 Found 438.3 |

### Example 7

### rac-trans or cis-1-(3-chlorophenethyl)-3-((4-(methylsulfonyl)phenoxy)methyl)piperidin-4-ol (Compound 27)

### rac-cis or trans-1-(3-chlorophenethyl)-3-((4-(methylsulfonyl)phenoxy)methyl)piperidin-4-ol (Compound 28)

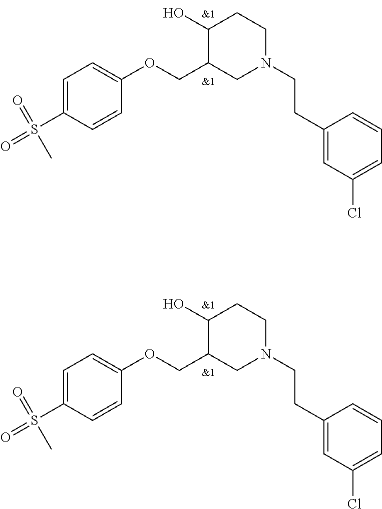 [SMILES: CS(=O)(=O)c1ccc(OCC2CN(CCc3cccc(Cl)c3)CCC2O)cc1 ; CS(=O)(=O)c1ccc(OCC2CN(CCc3cccc(Cl)c3)CCC2O)cc1]

### Step 1: tert-butyl 4-hydroxy-3-(hydroxymethyl)piperidine-1-carboxylate

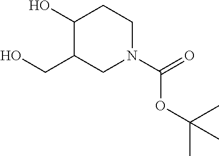 [SMILES: CC(C)(C)OC(=O)N1CCC(O)C(CO)C1]

To a 0° C. solution of 1-tert-butyl 3-ethyl 4-oxopiperidine-1,3-dicarboxylate (5.00 g, 18.4 mmol) in EtOH (55 mL) was added NaBH₄ (6.97 g, 184 mmol) slowly. The mixture was stirred at 0° C. for 5 h, and was then quenched with ice water (40 mL). After stirring for 30 min, the solution was extracted with EtOAc (40 mL×3). The combined organic layers were washed with brine (10 mL×2), dried over Na₂SO₄, filtered and concentrated in vacuo to give tert-butyl 4-hydroxy-3-(hydroxymethyl)piperidine-1-carboxylate, which was used in the next step without further purification. MS=176.0 [M-C₄H₇+H]⁺.

### Step 2: tert-butyl 4-hydroxy-3-((4-(methylsulfonyl)phenoxy)methyl)piperidine-1-carboxylate

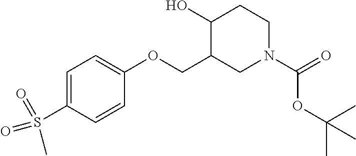 [SMILES: CC(C)(C)OC(=O)N1CCC(O)C(COc2ccc(S(C)(=O)=O)cc2)C1]

To a solution of tert-butyl 4-hydroxy-3-(hydroxymethyl)piperidine-1-carboxylate (2.80 g, 12.1 mmol) and 1-fluoro-4-methylsulfonyl-benzene (2.11 g, 12.1 mmol) in DMF (28 mL) was added K₂CO₃ (4.18 g, 30.3 mmol). The mixture was stirred at 100° C. for 15 h. After cooling to room temperature, the reaction mixture was quenched with water (50 mL) and extracted with EtOAc (30 mL×3). The combined organic layers were washed with brine (10 mL×2), dried over Na₂SO₄, filtered and concentrated in vacuo. The crude residue was purified by normal phase silica gel chromatography (Biotage 20 g cartridge, 0-65% EtOAc in petroleum ether) to give tert-butyl 4-hydroxy-3-((4-(methylsulfonyl)phenoxy)methyl)piperidine-1-carboxylate. MS=408.1 [M+Na]⁺.

### Step 3: 3-((4-(methylsulfonyl)phenoxy)methyl)piperidin-4-ol

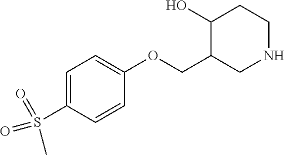 [SMILES: CS(=O)(=O)c1ccc(OCC2CNCCC2O)cc1]

A solution of tert-butyl 4-hydroxy-3-[(4-methylsulfonylphenoxy)methyl]piperidine-1-carboxylate (1.20 g, 3.11 mmol) in HCl/EtOAc (4 M, 15 mL) was stirred at room temperature for 2 h and then concentrated in vacuo to give 3-((4-(methylsulfonyl)phenoxy)methyl)piperidin-4-ol (HCl salt), which was used in the next step without further purification. MS=286.0 [M+H]⁺.

### Step 4: rac-trans or cis-1-(3-chlorophenethyl)-3-((4-(methylsulfonyl)phenoxy)methyl)piperidin-4-ol (Compound 27); rac-cis or trans-1-(3-chlorophenethyl)-3-((4-(methylsulfonyl)phenoxy)methyl)piperidin-4-ol (Compound 28)

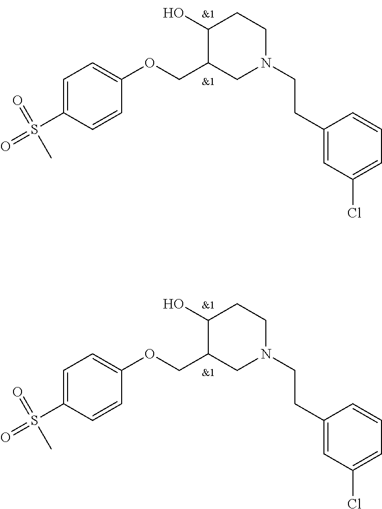 [SMILES: CS(=O)(=O)c1ccc(OCC2CN(CCc3cccc(Cl)c3)CCC2O)cc1 ; CS(=O)(=O)c1ccc(OCC2CN(CCc3cccc(Cl)c3)CCC2O)cc1]

To a solution of 3-((4-(methylsulfonyl)phenoxy)methyl)piperidin-4-ol (970 mg, 3.01 mmol, HCl salt) and 2-(3-chlorophenyl)acetaldehyde (559 mg, 3.62 mmol) in MeOH (10 mL) was added TEA (0.839 mL, 6.03 mmol) and AcOH (34.48 μL, 0.603 mmol). After stirring for 30 min, NaBH₃CN (568 mg, 9.04 mmol) was added. The mixture was stirred at room temperature for 10 h, then was quenched with ice water (10 mL). The mixture was concentrated to remove solvent and extracted with EtOAc (10 mL×3). The combined organic layers were washed with brine (5 mL), dried over Na₂SO₄, filtered and concentrated in vacuo. The crude residue was purified by reverse phase preparative HPLC (Waters Xbridge BEH C18 column, 30-50% MeCN/10 mM NH₄HCO₃ in water). The first eluting enantiomeric mixture of the title compound, Compound 27: ¹H NMR (400 MHz, DMSO-d₆): δ 7.86-7.81 (m, 2H), 7.33-7.14 (m, 6H), 4.81-4.78 (m, 1H), 4.26 (app dd, J=10.0 Hz, 3.2 Hz, 1H), 4.01 (app dd, J=9.6 Hz, 8.0 Hz, 1H), 3.48-3.36 (m, 1H), 3.15 (s, 3H), 3.02 (d, J=10.0 Hz, 1H), 2.88-2.86 (m, 1H), 2.7-2.71 (m, 2H), 2.62-2.58 (m, 1H), 2.31-2.22 (m, 1H), 2.05-1.95 (m, 2H), 1.88-1.82 (m, 2H), 1.50-1.41 (m, 1H). MS=424.3 [M+H]⁺. The second eluting enantiomeric mixture of the title compound, Compound 28: ¹H NMR (400 MHz, DMSO-d₆): δ 7.82 (d, J=8.8 Hz, 2H), 7.28-7.13 (m, 6H), 4.69 (s, 1H), 4.16-4.12 (m, 1H), 4.04-3.99 (m, 1H), 3.85 (s, 1H), 3.15 (s, 3H), 2.76-2.68 (m, 2H), 2.61-2.55 (m, 2H), 2.49-2.33 (m, 4H), 2.10-2.09 (m, 1H), 1.66-1.52 (m, 2H). MS=424.2 [M+H]⁺.

### Example 8

### (3S,4R) or (3R,4R) or (3S,4S) or (3R,4S)-1-(3-chlorophenethyl)-3-((4-(methylsulfonyl)phenoxy)methyl)piperidin-4-ol (Compound 29)

### (3R,4R) or (3S,4S) or (3R,4S) or (3S,4R)-1-(3-chlorophenethyl)-3-((4-(methylsulfonyl)phenoxy)methyl)piperidin-4-ol (Compound 30)

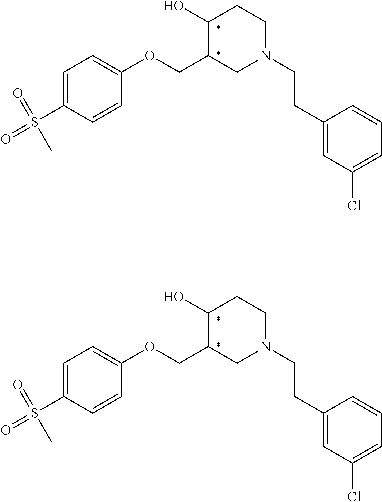 [SMILES: CS(=O)(=O)c1ccc(OCC2CN(CCc3cccc(Cl)c3)CCC2O)cc1 ; CS(=O)(=O)c1ccc(OCC2CN(CCc3cccc(Cl)c3)CCC2O)cc1]

A solution of rac-trans- or rac-cis-1-(3-chlorophenethyl)-3-((4-(methylsulfonyl)phenoxy)methyl)piperidin-4-ol (Compound 27, 0.035 g, 0.083 mmol) in MeOH (1 mL) was separated by preparative chiral SFC (DAICEL CHIRALPAK IG-3, 60% MeOH with 0.1% NH₄OH in CO₂). The first eluting enantiomer of the title compound, Compound 29: ¹H NMR (400 MHz, DMSO-d₆): δ 7.84-7.80 (m, 2H), 7.32-7.14 (m, 6H), 4.79 (d, J=5.2 Hz, 1H), 4.25 (app dd, J=10.0 Hz, 2.8 Hz, 1H), 4.01 (app t, J=8.4 Hz, 1H), 3.46-3.38 (m, 1H), 3.15 (s, 3H), 3.06-2.97 (m, 1H), 2.92-2.82 (m, 1H), 2.77-2.68 (m, 2H), 2.64-2.56 (m, 2H), 2.07-1.95 (m, 2H), 1.89-1.77 (m, 2H), 1.51-1.40 (m, 1H). MS=424.2 [M+H]⁺. The second eluting enantiomer of the title compound, Compound 30: ¹H NMR (400 MHz, DMSO-d₆): δ 7.84-7.82 (m, 2H), 7.30-7.14 (m, 6H), 4.79 (d, J=5.2 Hz, 1H), 4.25 (app dd, J=9.6 Hz, 2.8 Hz, 1H), 4.03-3.98 (m, 1H), 3.29 (s, 1H), 3.15 (s, 3H), 3.02 (d, J=10.4 Hz, 1H), 2.86 (d, J=10.4 Hz, 1H), 2.77-2.64 (m, 2H), 2.62-2.54 (m, 2H), 2.06-1.95 (m, 2H), 1.87-1.76 (m, 2H), 1.50-1.40 (m, 1H). MS=424.3 [M+H]⁺.

The following compounds in Table 15 were prepared according to procedures similar to those described for Compound 29 using the appropriate starting materials.

TABLE 15

| | | | | | Chiral | Exact |
|---|---|---|---|---|---|---|
| | Starting | | IUPAC | Chiral | Elution | Mass |
| No. | Material | Structure | Name | Column | Order | [M + H]⁺ |
| 31 | Compound 28 | 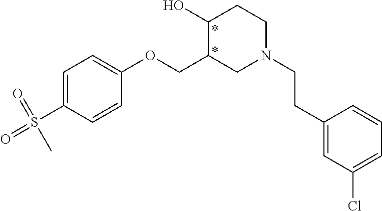 [SMILES: CS(=O)(=O)c1ccc(OCC2CN(CCc3cccc(Cl)c3)CCC2O)cc1] | (3R,4R) or (3S,4S) or (3R,4S) or (3S,4R)-1- (3- chlorophen- ethyl)-3-((4- (methylsulf- onyl)phenoxy) methyl) piperidin-4-ol | DAICEL CHIRAL PAK IG- 3 | 1st | Calc'd 424.1 Found 424.2 |
| 32 | Compound 28 | 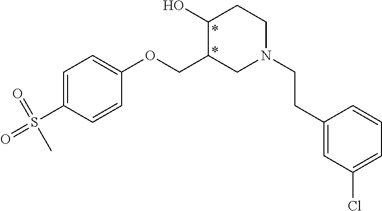 [SMILES: CS(=O)(=O)c1ccc(OCC2CN(CCc3cccc(Cl)c3)CCC2O)cc1] | (3S,4S) or (3R,4S) or (3S,4R) or (3R,4R)-1- (3- chlorophen- ethyl)-3-((4- (methylsulf- onyl)phenoxy) methyl) piperidin-4-ol | DAICEL CHIRAL PAK IG- 3 | 2nd | Calc'd 424.1 Found 424.3 |

### Example 9

### rac-trans or cis-1-(3-chlorophenethyl)-4-methoxy-3-((4-(methylsulfonyl)phenoxy) methyl)piperidine (Compound 33)

### rac-cis or trans-1-(3-chlorophenethyl)-4-methoxy-3-((4-(methylsulfonyl)phenoxy) methyl)piperidine (Compound 34)

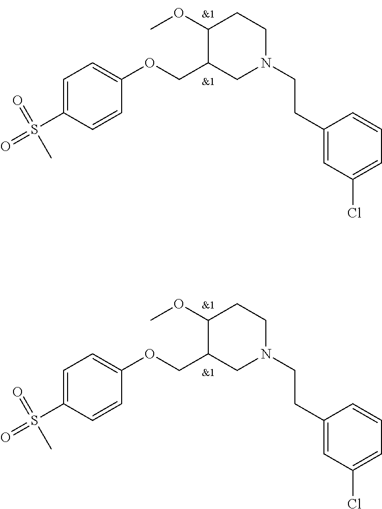 [SMILES: COC1CCN(CCc2cccc(Cl)c2)CC1COc1ccc(S(C)(=O)=O)cc1 ; COC1CCN(CCc2cccc(Cl)c2)CC1COc1ccc(S(C)(=O)=O)cc1]

### Step 1: tert-butyl 3-(hydroxymethyl)-4-methoxypiperidine-1-carboxylate

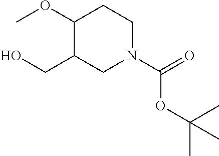 [SMILES: COC1CCN(C(=O)OC(C)(C)C)CC1CO]

To a 0° C. solution of 1-(tert-butoxycarbonyl)-4-methoxypiperidine-3-carboxylic acid (900 mg, 3.47 mmol) in THF (10 mL) was added BH₃·THF (1.0 M, 6.94 mL) dropwise. The mixture was warmed to room temperature and stirred for 2 h. The reaction mixture was cooled to 0° C. and quenched with MeOH (20 mL), and then the adjusted pH=7 by the dropwise addition of aqueous NaHCO₃. The mixture was extracted with EtOAc (50 mL×3), and the combined organic layers were dried over Na₂SO₄, filtered and concentrated in vacuo to give tert-butyl 3-(hydroxymethyl)-4-methoxypiperidine-1-carboxylate, which was used in the next step without further purification. MS=190.2 [M-C₄H₇+H]⁺.

### Step 2: tert-butyl 4-methoxy-3-((4-(methylsulfonyl)phenoxy)methyl)piperidine-1-carboxylate

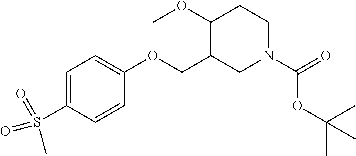 [SMILES: COC1CCN(C(=O)OC(C)(C)C)CC1COc1ccc(S(C)(=O)=O)cc1]

To a mixture of 1-fluoro-4-methylsulfonyl-benzene (508 mg, 2.91 mmol) and tert-butyl 3-(hydroxymethyl)-4-methoxy-piperidine-1-carboxylate (715 mg, 2.91 mmol) in DMF (10 mL) was added Cs₂CO₃ (2.85 g, 8.74 mmol). The mixture was stirred at 100° C. for 15 h, then was cooled to room temperature. The reaction mixture was diluted with water (30 mL), and then extracted with EtOAc (50 mL×3). The combined organic layers were dried over Na₂SO₄, filtered and concentrated in vacuo. The crude residue was purified by normal phase silica gel chromatography (Biotage 20 g cartridge, 0-50% EtOAc in petroleum ether) to give tert-butyl 4-methoxy-3-((4-(methylsulfonyl)phenoxy)methyl)piperidine-1-carboxylate. MS=344.2 [M-C₄H₇+H]⁺.

### Step 3: 4-methoxy-3-((4-(methylsulfonyl)phenoxy)methyl)piperidine

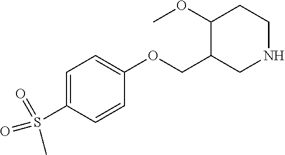 [SMILES: COC1CCNCC1COc1ccc(S(C)(=O)=O)cc1]

To a 0° C. solution of tert-butyl 4-methoxy-3-((4-(methylsulfonyl)phenoxy)methyl)piperidine-1-carboxylate (790 mg, 1.98 mmol) in EtOAc was added HCl/EtOAc (4 M, 10 mL). The mixture was warmed to room temperature and stirred for 1 h. The reaction mixture was filtered, and the filter cake was washed with petroleum ether (10 mL×3) and concentrated under reduced pressure to give 4-methoxy-3-((4-(methylsulfonyl)phenoxy)methyl)piperidine (HCl salt), which was used in the next step without further purification. MS=300.1 [M+H]⁺.

### Step 4: rac-trans or cis-1-(3-chlorophenethyl)-4-methoxy-3-((4-(methylsulfonyl)phenoxy) methyl)piperidine (Compound 33); rac-cis or trans-1-(3-chlorophenethyl)-4-methoxy-3-((4-(methylsulfonyl)phenoxy) methyl)piperidine (Compound 34)

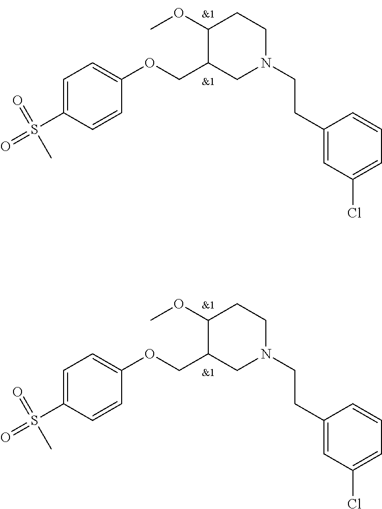 [SMILES: COC1CCN(CCc2cccc(Cl)c2)CC1COc1ccc(S(C)(=O)=O)cc1 ; COC1CCN(CCc2cccc(Cl)c2)CC1COc1ccc(S(C)(=O)=O)cc1]

To a mixture of 4-methoxy-3-((4-(methylsulfonyl)phenoxy)methyl)piperidine (527 mg, 1.57 mmol, HCl salt) and 2-(3-chlorophenyl)acetaldehyde (485 mg, 3.14 mmol) in MeOH (8 mL) was added TEA (218 μL, 1.57 mmol) and HOAc (19.1 μL, 0.333 mmol). After stirring at room temperature for 3 h, NaBH₃CN (197 mg, 3.14 mmol) was added. The mixture was stirred at room temperature for 27 h, then was quenched with water (1 mL) and concentrated in vacuo. The residue was purified by reverse phase preparative HPLC (Kromasil C18 column, 45-65% MeCN/10 mM NH₄HCO₃ in water). The first eluting enantiomeric mixture of the title compound, Compound 33: ¹H NMR (400 MHz, DMSO-d₆): δ 7.84 (d, J=2.0 Hz, 2H), 7.31-7.15 (m, 6H), 4.19-403 (m, 2H), 3.25 (s, 3H), 3.24 (s, 3H), 3.16-3.13 (m, 1H), 2.98-2.84 (m, 2H), 2.75-2.50 (m, 4H), 2.12-2.05 (m, 4H), 1.37-1.35 (m, 1H). MS=438.2 [M+H]⁺. The second eluting enantiomeric mixture of the title compound, Compound 34: ¹H NMR (400 MHz, DMSO-d₆): δ 7.83 (d, J=8.8 Hz, 2H), 7.28-7.14 (m, 6H), 4.10-4.02 (m, 2H), 3.48 (s, 1H), 3.31 (s, 3H), 3.25 (s, 3H), 2.73-2.67 (m, 2H), 2.57-2.52 (m, 6H), 2.33-2.26 (m, 1H), 1.76-1.60 (m, 2H). MS=438.2 [M+H]⁺.

### Example 10

### (3S,4R) or (3R,4R) or (3S,4S) or (3R,4S)-1-(3-chlorophenethyl)-4-methoxy-3-((4-(methylsulfonyl)phenoxy)methyl)piperidine (Compound 35)

### (3R,4R) or (3S,4S) or (3R,4S) or (3S,4R)-1-(3-chlorophenethyl)-4-methoxy-3-((4-(methylsulfonyl)phenoxy)methyl)piperidine (Compound 36)

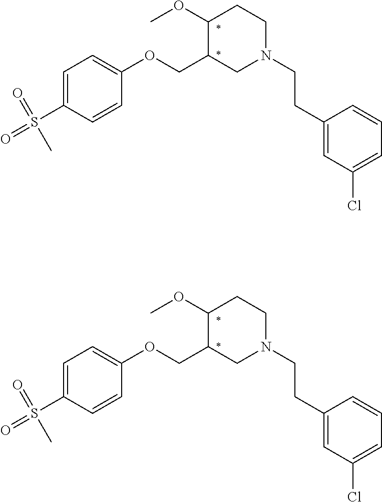 [SMILES: COC1CCN(CCc2cccc(Cl)c2)CC1COc1ccc(S(C)(=O)=O)cc1 ; COC1CCN(CCc2cccc(Cl)c2)CC1COc1ccc(S(C)(=O)=O)cc1]

Rac-trans or cis-1-(3-chlorophenethyl)-4-methoxy-3-((4-(methylsulfonyl)phenoxy) methyl)piperidine (Compound 33, 120 mg, 0.275 mmol) was separated by preparative chiral SFC (DAICEL CHIRALPAK AD-3, 50% MeOH with 0.1% NH₄OH in CO₂). The first eluting enantiomer of the title compound, Compound 35: ¹H NMR (400 MHz, DMSO-d₆): δ 7.84 (d, J=8.8 Hz, 2H), 7.30-7.15 (m, 6H), 4.19-4.16 (m, 1H), 4.07-4.03 (m, 1H), 3.25 (s, 3H), 3.15 (s, 3H), 3.08 (s, 1H), 2.98-2.85 (m, 2H), 2.75-2.53 (m, 4H), 2.10-1.97 (m, 4H), 1.38-1.35 (m, 1H). MS=438.2 [M+H]⁺. The second eluting enantiomer of the title compound, Compound 36: ¹H NMR (400 MHz, DMSO-d₆): δ 7.83 (d, J=8.4 Hz, 2H), 7.31-7.15 (m, 6H), 4.19-4.03 (m, 2H), 3.25 (s, 3H), 3.15 (s, 3H), 3.12 (s, 1H), 2.99-2.85 (m, 2H), 2.75-2.71 (m, 4H), 2.10-1.98 (m, 4H), 1.37-1.23 (m, 1H). MS=438.2 [M+H]⁺.

The following compounds in Table 16 were prepared according to procedures similar to those described for Compounds 35 and 36 using the appropriate starting materials.

TABLE 16

| | | | | | Chiral | Exact |
|---|---|---|---|---|---|---|
| | Starting | | | Chiral | Elution | Mass |
| No. | Material | Structure | IUPAC Name | Column | Order | [M + H]⁺ |
| 37 | Compound 34 | 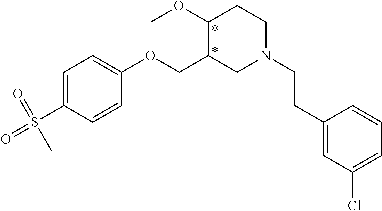 [SMILES: COC1CCN(CCc2cccc(Cl)c2)CC1COc1ccc(S(C)(=O)=O)cc1] | (3R,4R) or (3S,4S) or (3R,4S) or (3S,4R)-1-(3- chlorophenethyl)- 4-methoxy-3- ((4- (methylsulfonyl) phenoxy)methyl) piperidine | DAICEL CHIRAL PAK AD- 3 | 1st | Calc'd 438.1 Found 438.2 |
| 38 | Compound 34 | 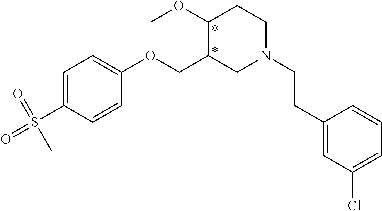 [SMILES: COC1CCN(CCc2cccc(Cl)c2)CC1COc1ccc(S(C)(=O)=O)cc1] | (3S,4S) or (3R,4S) or (3 S,4R) or (3R,4R)- 1-(3- chlorophenethyl)- 4-methoxy-3- ((4- (methylsulfonyl) phenoxy)methyl) piperidine | DAICEL CHIRAL PAK AD- 3 | 2nd | Calc'd 438.1 Found 438.2 |

### Example 11

### 1-(3-chlorophenethyl)-3-(4-(methylsulfonyl)phenethyl)piperidine (Compound 39)

### (S) or (R)-1-(3-chlorophenethyl)-3-(4-(methylsulfonyl)phenethyl)piperidine (Compound 40)

### (R) or (S)-1-(3-chlorophenethyl)-3-(4-(methylsulfonyl)phenethyl)piperidine (Compound 41)

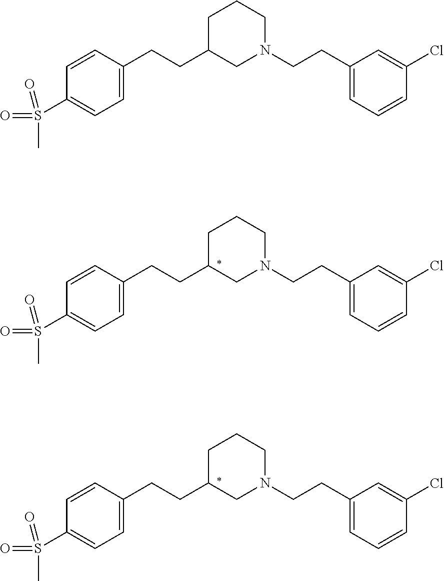 [SMILES: CS(=O)(=O)c1ccc(CCC2CCCN(CCc3cccc(Cl)c3)C2)cc1 ; CS(=O)(=O)c1ccc(CCC2CCCN(CCc3cccc(Cl)c3)C2)cc1 ; CS(=O)(=O)c1ccc(CCC2CCCN(CCc3cccc(Cl)c3)C2)cc1]

### Step 1: tert-butyl 5-(((trifluoromethyl)sulfonyl)oxy)-3,4-dihydropyridine-1(2H)-carboxylate

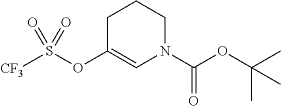 [SMILES: CC(C)(C)OC(=O)N1C=C(OS(=O)(=O)C(F)(F)F)CCC1]

To a solution of tert-butyl 3-oxopiperidine-1-carboxylate (2.00 g, 10.0 mmol) in THF (20 mL) at −78° C. was added LiHMDS (6.02 mL, 2 M, 12.1 mmol) dropwise. After 30 min, 1,1,1-trifluoro-N-phenyl-N-(trifluoromethylsulfonyl)methanesulfonamide (4.30 g, 12.1 mmol) was added. The mixture was warmed to room temperature and stirred for 2 h, and then quenched with aqueous NH₄Cl (10 mL) and extracted with EtOAc (30 mL×3). The combined organic layers were dried over Na₂SO₄, filtered and concentrated in vacuo. The crude residue was purified by normal phase silica gel chromatography (Biotage 10 g cartridge, 0-100% EtOAc in petroleum ether) to give tert-butyl 5-(((trifluoromethyl)sulfonyl)oxy)-3,4-dihydropyridine-1(2H)-carboxylate. MS=354.0 [M+Na]⁺.

### Step 2: (E)-tert-butyl 5-(4-(methylsulfonyl)styryl)-3,4-dihydropyridine-1(2H)-carboxylate

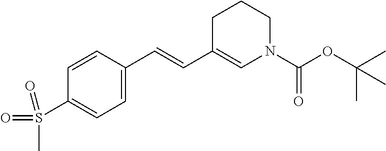 [SMILES: CC(C)(C)OC(=O)N1C=C(/C=C/c2ccc(S(C)(=O)=O)cc2)CCC1]

A solution of tert-butyl 5-(((trifluoromethyl)sulfonyl)oxy)-3,4-dihydropyridine-1(2H)-carboxylate (682 mg, 2.06 mmol), 1-(methylsulfonyl)-4-vinylbenzene (250 mg, 1.37 mmol), TEA (573 μL, 4.12 mmol), PPh₃ (14.39 mg, 0.055 mmol), and Pd(OAc)₂ (6.16 mg, 0.027 mmol) in DMF (5 mL) was sparged with N₂, then stirred at 60° C. for 5 h. The reaction mixture was cooled to room temperature, filtered, and the filtrate was concentrated in vacuo. The crude residue was purified by normal phase silica gel chromatography (Biotage 10 g cartridge, 0-10% EtOAc in petroleum ether) to give (E)-tert-butyl 5-(4-(methylsulfonyl)styryl)-3,4-dihydropyridine-1(2H)-carboxylate. MS=364.1 [M+H]⁺.

### Step 3: tert-butyl 3-(4-(methylsulfonyl)phenethyl)piperidine-1-carboxylate

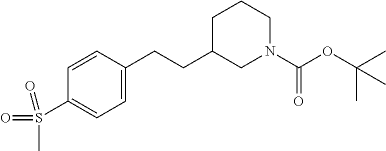 [SMILES: CC(C)(C)OC(=O)N1CCCC(CCc2ccc(S(C)(=O)=O)cc2)C1]

To a solution of (E)-tert-butyl 5-(4-(methylsulfonyl)styryl)-3,4-dihydropyridine-1(2H)-carboxylate (230 mg, 0.633 mmol) in MeOH (3 mL) was added Pd/C (50 mg, 10% by weight). The mixture was purged with H₂ twice and stirred under H₂ (15 Psi) at room temperature for 3 h. The reaction solution was filtered through celite, and the filtrate was concentrated in vacuo to give tert-butyl 3-(4-(methylsulfonyl)phenethyl)piperidine-1-carboxylate, which was used in the next step without further purification. MS=390.1 [M+Na]⁺.

### Step 4: 3-(4-(methylsulfonyl)phenethyl)piperidine

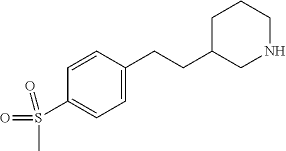 [SMILES: CS(=O)(=O)c1ccc(CCC2CCCNC2)cc1]

A solution of tert-butyl 3-(4-(methylsulfonyl)phenethyl)piperidine-1-carboxylate (250 mg, 0.680 mmol) in HCl/EtOAc (4 M, 5 mL) was stirred at room temperature for 1 h. The reaction was concentrated in vacuo to give 3-(4-(methylsulfonyl)phenethyl)piperidine (HCl salt), which was used in the next step without further purification. MS=268.3 [M+H]⁺.

### Step 5: 1-(3-chlorophenethyl)-3-(4-(methylsulfonyl)phenethyl)piperidine (Compound 39)

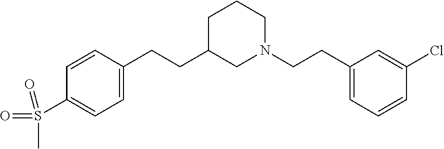 [SMILES: CS(=O)(=O)c1ccc(CCC2CCCN(CCc3cccc(Cl)c3)C2)cc1]

A solution of 3-(4-(methylsulfonyl)phenethyl)piperidine (0.23 g, 0.757 mmol, HCl salt) and 2-(3-chlorophenyl)acetaldehyde (117 mg, 0.757 mmol) in MeOH (5 mL) was stirred at room temperature for 1 h, and then NaBH₃CN (95.1 mg, 1.51 mmol) was added. After stirring for an additional 15 h at room temperature, the mixture was quenched with water (1 mL) and mixture was adjusted to pH 7 via the dropwise addition of aqueous 2 M HCl. The mixture was concentrated under reduced pressure and MeOH (3 mL) was added to the residue. The crude residue was purified by reverse phase preparative HPLC (Waters Xbridge BEH C18 column, 35-65% MeCN/10 mM NH₄HCO₃ in water) to give 1-(3-chlorophenethyl)-3-(4-(methylsulfonyl)phenethyl)piperidine (Compound 39). ¹H NMR (400 MHz, DMSO-d₆): δ 7.82 (d, J=8.4 Hz, 2H), 7.47 (d, J=8.4 Hz, 2H), 7.32-7.15 (m, 4H), 3.31 (s, 3H), 2.80-2.68 (m, 6H), 2.48-2.44 (m, 2H), 1.99-1.90 (m, 1H), 1.74-1.37 (m, 7H), 0.94-0.86 (m, 1H). MS=406.2 [M+H]⁺.

### Step 6: (S) or (R)-1-(3-chlorophenethyl)-3-(4-(methylsulfonyl)phenethyl)piperidine (Compound 40); (R) or (S)-1-(3-chlorophenethyl)-3-(4-(methylsulfonyl)phenethyl)piperidine (Compound 41)

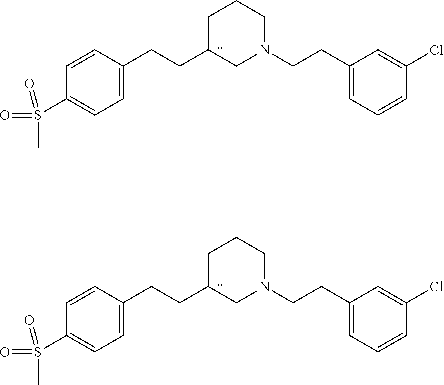 [SMILES: CS(=O)(=O)c1ccc(CCC2CCCN(CCc3cccc(Cl)c3)C2)cc1 ; CS(=O)(=O)c1ccc(CCC2CCCN(CCc3cccc(Cl)c3)C2)cc1]

1-(3-chlorophenethyl)-3-(4-(methylsulfonyl)phenethyl)piperidine (Compound 39, 250 mg, 0.616 mmol) was separated by preparative chiral SFC (DAICEL CHIRALPAK AD-3, 50% MeOH with 0.1% NH₄OH in CO₂). The first eluting enantiomer of the title compound, Compound 40: ¹H NMR (400 MHz, DMSO-d₆): δ 7.82 (d, J=8.4 Hz, 2H), 7.47 (d, J=8.4 Hz, 2H), 7.32-7.17 (m, 4H), 3.18 (s, 3H), 2.84-2.68 (m, 6H), 2.48-2.44 (m, 2H), 1.93-1.90 (m, 1H), 1.74-1.41 (m, 7H), 0.92-0.89 (m, 1H). MS=406.1 [M+H]⁺. The second eluting enantiomer of the title compound, Compound 41: ¹H NMR (400 MHz, DMSO-d₆): δ 7.82 (d, J=8.4 Hz, 2H), 7.47 (d, J=8.4 Hz, 2H), 7.31-7.19 (m, 4H), 3.18 (s, 3H), 2.85-2.68 (m, 6H), 2.48-2.44 (m, 2H), 1.99-1.90 (m, 1H), 1.74-1.41 (m, 7H), 0.92-0.89 (m, 1H). MS=406.1 [M+H]⁺.

### Example 12

### 1-(3-chlorophenethyl)-2,2-dimethyl-4-((4-(methylsulfonyl)phenoxy) methyl) pyrrolidine (Compound 42)

### (S) or (R)-1-(3-chlorophenethyl)-2,2-dimethyl-4-((4-(methylsulfonyl)phenoxy) methyl) pyrrolidine (Compound 43)

### (R) or (S)-1-(3-chlorophenethyl)-2,2-dimethyl-4-((4-(methylsulfonyl)phenoxy)methyl)pyrrolidine (Compound 44)

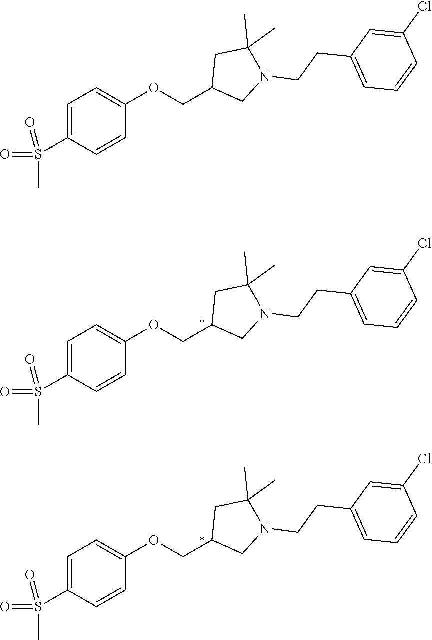 [SMILES: CC1(C)CC(COc2ccc(S(C)(=O)=O)cc2)CN1CCc1cccc(Cl)c1 ; CC1(C)CC(COc2ccc(S(C)(=O)=O)cc2)CN1CCc1cccc(Cl)c1 ; CC1(C)CC(COc2ccc(S(C)(=O)=O)cc2)CN1CCc1cccc(Cl)c1]

### Step 1: tert-butyl 2,2-dimethyl-4-((4-(methylsulfonyl)phenoxy)methyl)pyrrolidine-1-carboxylate

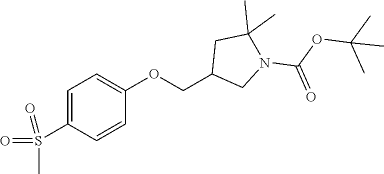 [SMILES: CC(C)(C)OC(=O)N1CC(COc2ccc(S(C)(=O)=O)cc2)CC1(C)C]

A mixture of tert-butyl 4-(hydroxymethyl)-2,2-dimethylpyrrolidine-1-carboxylate (500 mg, 2.18 mmol), 1-fluoro-4-methylsulfonyl-benzene (380 mg, 2.18 mmol) and Cs₂CO₃ (2.13 g, 6.54 mmol) in DMF (5 mL) was stirred at 100° C. for 16 h. The reaction mixture was cooled to room temperature, quenched by the addition of water (15 mL), and then extracted with EtOAc (15 mL×3). The combined organic layers were washed with water (15 mL×3), dried over Na₂SO₄, filtered and concentrated in vacuo. The crude residue was purified by normal phase silica gel chromatography (Biotage 12 g cartridge, 0-45% EtOAc in petroleum ether) to give tert-butyl 2,2-dimethyl-4-((4-(methylsulfonyl)phenoxy)methyl)pyrrolidine-1-carboxylate. MS=406.2 [M+Na]⁺.

### Step 2: 2,2-dimethyl-4-((4-(methylsulfonyl)phenoxy)methyl)pyrrolidine

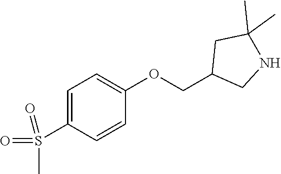 [SMILES: CC1(C)CC(COc2ccc(S(C)(=O)=O)cc2)CN1]

To a solution of tert-butyl 2,2-dimethyl-4-((4-(methylsulfonyl)phenoxy)methyl)pyrrolidine-1-carboxylate (0.85 g, 2.22 mmol) in DCM (10 mL) was added TFA (4 mL). The mixture was stirred at room temperature for 1.5 h, and then was quenched by the addition of water (15 mL). The mixture was concentrated under reduced pressure to remove DCM, and the resulting aqueous layer was extracted with EtOAc (15 mL×5). The combined organic layers were dried over Na₂SO₄, filtered and concentrated under reduced pressure to give 2,2-dimethyl-4-((4-(methylsulfonyl)phenoxy)methyl)pyrrolidine (TFA salt). MS=284.1 [M+H]⁺.

### Step 3: 1-(3-chlorophenethyl)-2,2-dimethyl-4-((4-(methylsulfonyl)phenoxy)methyl) pyrrolidine (Compound 42)

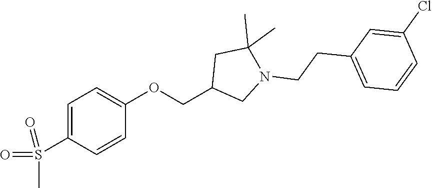 [SMILES: CC1(C)CC(COc2ccc(S(C)(=O)=O)cc2)CN1CCc1cccc(Cl)c1]

To a solution of 2-(3-chlorophenyl)acetaldehyde (982 mg, 6.35 mmol) and TEA (663 μL, 4.76 mmol) in MeOH (5 mL) was added 2,2-dimethyl-4-((4-(methylsulfonyl)phenoxy)methyl)pyrrolidine (450 mg, 1.18 mmol, TFA salt). After stirring at room temperature for 30 min, the reaction was cooled to 0° C. and NaBH₃CN (299 mg, 4.76 mmol) was added. The resulting mixture was stirred at room temperature for 24 h, then was quenched by the addition of water (0.5 mL) and concentrated in vacuo. The crude residue was purified by reverse phase preparative HPLC (Waters Xbridge BEH C18 column, 50-80% MeCN/10 mM NH₄HCO₃ in water) to give 1-(3-chlorophenethyl)-2,2-dimethyl-4-((4-(methylsulfonyl)phenoxy)methyl)pyrrolidine (Compound 42). ¹H NMR (400 MHz, DMSO-d₆): δ 7.83 (d, J=8.8 Hz, 2H), 7.30-7.14 (m, 6H), 3.95 (d, J=7.2 Hz, 2H), 3.15 (s, 3H), 2.83-2.75 (m, 2H), 2.70-2.62 (m, 3H), 2.60-2.54 (m, 1H), 2.47-2.41 (m, 1H), 1.81 (app dd, J=12.4 Hz, 9.2 Hz, 1H), 1.34 (app dd, J=12.4 Hz, 6.8 Hz, 1H), 0.95 (s, 3H), 0.89 (s, 3H). MS=422.1 [M+H]⁺.

### Step 4: (S) or (R)-1-(3-chlorophenethyl)-2,2-dimethyl-4-((4-(methylsulfonyl)phenoxy) methyl) pyrrolidine (Compound 43); (R) or (S)-1-(3-chlorophenethyl)-2,2-dimethyl-4-((4-(methylsulfonyl)phenoxy)methyl)pyrrolidine (Compound 44)

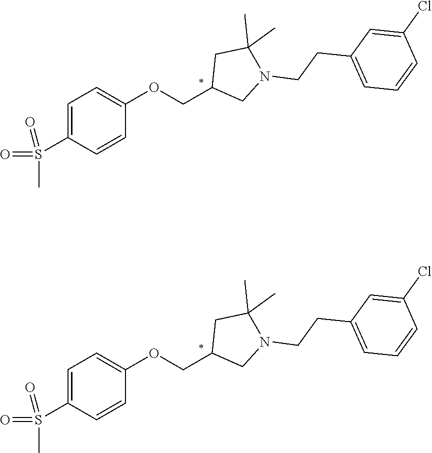 [SMILES: CC1(C)CC(COc2ccc(S(C)(=O)=O)cc2)CN1CCc1cccc(Cl)c1 ; CC1(C)CC(COc2ccc(S(C)(=O)=O)cc2)CN1CCc1cccc(Cl)c1]

1-(3-chlorophenethyl)-2,2-dimethyl-4-((4-(methylsulfonyl)phenoxy)methyl)pyrrolidine (Compound 42, 140 g, 332 mmol) was separated by preparative chiral SFC (DAICEL CHIRALPAK AD-3, 38% MeOH with 0.1% NH₄OH in CO₂). The first eluting enantiomer of the title compound, Compound 43: ¹H NMR (400 MHz, DMSO-d₆): δ 7.83 (d, J=8.8 Hz, 2H), 7.30-7.14 (m, 6H), 3.95 (d, J=7.2 Hz, 2H), 3.15 (s, 3H), 2.83-2.76 (m, 2H), 2.70-2.62 (m, 3H), 2.55-2.54 (m, 1H), 2.46-2.45 (m, 1H), 1.81 (app dd, J=12.0 Hz, 9.2 Hz, 1H), 1.34 (app dd, J=12.4 Hz, 7.2 Hz, 1H), 0.94 (s, 3H), 0.89 (s, 3H). MS=422.1 [M+H]⁺. The second eluting enantiomer of the title compound, Compound 44: ¹H NMR (400 MHz, DMSO-d₆): δ 7.83 (d, J=8.8 Hz, 2H), 7.30-7.14 (m, 6H), 3.95 (d, J=7.2 Hz, 2H), 3.15 (s, 3H), 2.81-2.80 (m, 2H), 2.68-2.64 (m, 3H), 2.56-2.54 (m, 1H), 2.46-2.45 (m, 1H), 1.81 (app dd, J=12.4 Hz 9.2 Hz, 1H), 1.34 (d, J=12.4 Hz, 7.2 Hz, 1H), 0.95 (s, 3H), 0.89 (s, 3H). MS=422.1 [M+H]⁺.

### Example 13

### rac-trans-N-(4-((1-(3-chlorophenethyl)-4-methylpyrrolidin-3-yl)methoxy)phenyl)-N-methylmethanesulfonamide (Compound 45)

### N-(4-(((3S,4S) or (3R,4R)-1-(3-chlorophenethyl)-4-methylpyrrolidin-3-yl)methoxy)phenyl)-N-methylmethanesulfonamide (Compound 46)

### N-(4-(((3R,4R) or (3S,4S)-1-(3-chlorophenethyl)-4-methylpyrrolidin-3-yl)methoxy)phenyl)-N-methylmethanesulfonamide (Compound 47)

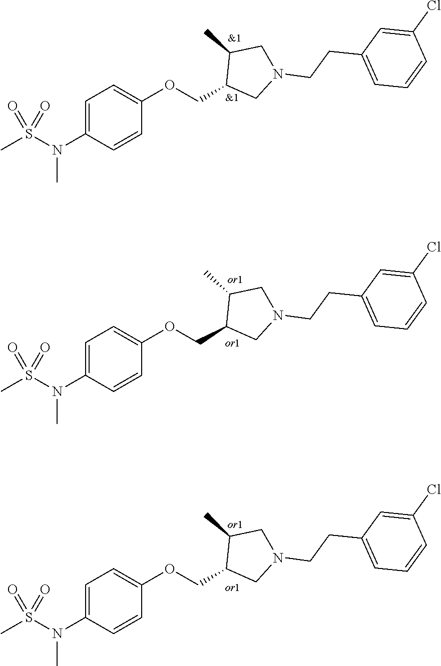 [SMILES: C[C@H]1CN(CCc2cccc(Cl)c2)C[C@@H]1COc1ccc(N(C)S(C)(=O)=O)cc1 ; C[C@@H]1CN(CCc2cccc(Cl)c2)C[C@H]1COc1ccc(N(C)S(C)(=O)=O)cc1 ; C[C@H]1CN(CCc2cccc(Cl)c2)C[C@@H]1COc1ccc(N(C)S(C)(=O)=O)cc1]

### Step 1: rac-trans-tert-butyl-3-methyl-4-((4-(N-methylmethylsulfonamido)phenoxy)methyl)pyrrolidine-1-carboxylate

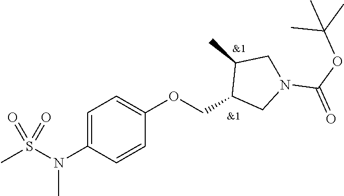 [SMILES: C[C@H]1CN(C(=O)OC(C)(C)C)C[C@@H]1COc1ccc(N(C)S(C)(=O)=O)cc1]

To a solution of N-(4-bromophenyl)-N-methylmethanesulfonamide (300 mg, 1.14 mmol), copper(I) iodide (11 mg, 0.057 mmol), cesium carbonate (0.554 g, 1.704 mmol), and 3,4,7,8-tetramethyl-1,10-phenanthroline (27 mg, 0.11 mmol) in toluene (1.62 mL) was added rac-trans-tert-butyl-3-(hydroxymethyl)-4-methylpyrrolidine-1-carboxylate (0.245 g, 1.14 mmol). The mixture was sparged with nitrogen for 1 min and sealed. The reaction was heated to 100° C. for 16 h. After cooling to room temperature, the mixture was diluted with EtOAc (10 mL) and filtered, solids were washed with EtOAc (10 mL×2), and the filtrate was concentrated in vacuo. The crude residue was dissolved in EtOAc (10 mL) and washed with aqueous 20% citric acid (10 mL). The aqueous layer was extracted with EtOAc (10 mL) and the combined organic layers were dried over Na₂SO₄, filtered and concentrated in vacuo. The crude residue was purified by normal phase silica gel chromatography (Biotage 25 g cartridge, 0-7% MeOH in DCM) to give rac-trans-tert-butyl-3-methyl-4-((4-(N-methylmethylsulfonamido)phenoxy)methyl)pyrrolidine-1-carboxylate. MS=421.1 [M+Na]⁺.

### Step 2: rac-trans-N-methyl-N-(4-((−4-methylpyrrolidin-3-yl)methoxy)phenyl)methanesulfonamide

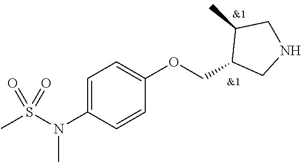 [SMILES: C[C@H]1CNC[C@@H]1COc1ccc(N(C)S(C)(=O)=O)cc1]

rac-trans-tert-butyl-3-methyl-4-((4-(N-methylmethylsulfonamido)phenoxy)methyl)pyrrolidine-1-carboxylate was dissolved in 20% TFA in DCM (3 mL) and allowed to stir at room temperature for 1 h. The reaction was concentrated in vacuo. The crude residue was dissolved in water (10 mL) and washed with DCM (10 mL). The aqueous layer was adjusted to pH=10 with the dropwise addition of 1 M NaOH. The aqueous layer was extracted with DCM (10 mL×2), and the combined organic layers were dried over Na₂SO₄, filtered and concentrated in vacuo to give rac-trans-N-methyl-N-(4-((4-methylpyrrolidin-3-yl)methoxy)phenyl)methanesulfonamide, which was taken to the next step without purification. MS=298.8 [M+H]⁺.

### Step 3: rac-trans-N-(4-((1-(3-chlorophenethyl)-4-methylpyrrolidin-3-yl)methoxy)phenyl)-N-methylmethanesulfonamide (Compound 45)

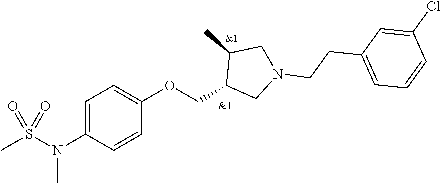 [SMILES: C[C@H]1CN(CCc2cccc(Cl)c2)C[C@@H]1COc1ccc(N(C)S(C)(=O)=O)cc1]

To a solution of rac-trans-tert-butyl-3-methyl-4-((4-(N methylmethylsulfonamido)phenoxy)methyl)pyrrolidine-1-carboxylate (54 mg, 0.18 mmol) and K₂CO₃ (0.10 g, 0.72 mmol) in MeCN (0.9 mL) was added 1-(2-bromoethyl)-3-chlorobenzene (0.048 g, 0.22 mmol). The mixture was heated to 70° C. and allowed to stir for 12 h. After cooling to the reaction to room temperature, the mixture was concentrated in vacuo. Water (10 mL) was added and the mixture was extracted with EtOAc (10 mL×3). The combined organic layers were dried over Na₂SO₄, filtered and concentrated in vacuo. The crude residue was purified by reverse phase preparative HPLC (Phenomenex Kinetex C18 column, 0-40% MeCN/water with 0.1% formic acid modifier) to give rac-trans-N-(4-((1-(3-chlorophenethyl)-4-methylpyrrolidin-3-yl)methoxy)phenyl)-N-methylmethanesulfonamide (Compound 45)¹H NMR (500 MHz, CDCl₃): δ 7.24-7.20 (m, 2H), 7.17-7.10 (m, 3H), 7.04-7.02 (m, 1H), 6.84-6.79 (m, 2H), 3.94-3.84 (m, 2H), 3.21 (s, 3H), 3.16-3.11 (m, 1H), 2.96-2.89 (m, 2H), 2.88-2.77 (m, 4H), 2.76 (s, 3H), 2.37 (t, J=9.1 Hz, 1H), 2.27-2.18 (m, 1H), 2.18-2.07 (m, 1H), 1.10 (d, J=6.7 Hz, 3H). MS=437.1 [M+H]⁺.

### Step 4: N-(4-(((3S,4S) or (3R,4R)-1-(3-chlorophenethyl)-4-methylpyrrolidin-3-yl)methoxy)phenyl)-N-methylmethanesulfonamide (Compound 46); N-(4-(((3R,4R) or (3S,4S)-1-(3-chlorophenethyl)-4-methylpyrrolidin-3-yl)methoxy)phenyl)-N-methylmethanesulfonamide (Compound 47)

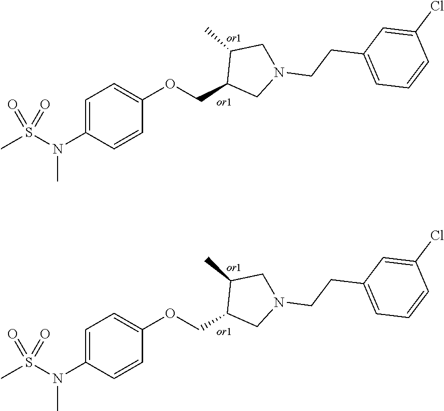 [SMILES: C[C@@H]1CN(CCc2cccc(Cl)c2)C[C@H]1COc1ccc(N(C)S(C)(=O)=O)cc1 ; C[C@H]1CN(CCc2cccc(Cl)c2)C[C@@H]1COc1ccc(N(C)S(C)(=O)=O)cc1]

rac-trans-N-(4-((1-(3-chlorophenethyl)-4-methylpyrrolidin-3-yl)methoxy)phenyl)-N-methylmethanesulfonamide (Compound 45, 26.2 mg, 0.060 mmol) was separated by preparative chiral SFC (Chiralcel OD-H column, 30% isopropanol with 0.25% isopropylamine in CO₂). The first eluting enantiomer of the title compound, Compound 46: ¹H NMR (500 MHz, DMSO-d₆): δ 7.34-7.16 (m, 6H), 6.98-6.92 (m, 2H), 3.94-3.91 (m, 1H), 3.87-3.84 (m, 1H), 3.18 (s, 3H), 2.90 (s, 3H), 2.85 (app dd, J=8.8, 7.3 Hz, 1H), 2.74-2.71 (m, 2H), 2.66-2.55 (m, 4H), 2.11-2.05 (m, 1H), 2.05-2.04 (m, 1H), 1.94-1.88 (m, 1H), 1.06 (d, J=6.8 Hz, 3H). MS=437.1 [M+H]⁺. The second eluting enantiomer of the title compound, Compound 47: ¹H NMR (500 MHz, DMSO-d₆): δ 7.36-7.15 (m, 6H), 6.98-6.92 (m, 2H), 3.94-3.91 (m, 1H), 3.87-3.84 (m, 1H), 3.18 (s, 3H), 2.90 (s, 3H), 2.84 (app dd, J=8.7, 7.3 Hz, 1H), 2.74-2.71 (m, 2H), 2.66-2.53 (m, 4H), 2.12-2.01 (m, 2H), 1.94-1.88 (m, 1H), 1.06 (d, J=6.7 Hz, 3H). MS=437.1 [M+H]⁺.

The following compounds in Table 17 were prepared according to procedures similar to steps 1-3 described for Compound 45 using the appropriate starting materials.

TABLE 17

| | | | Exact Mass |
|---|---|---|---|
| No. | Structure | IUPAC Name | [M + H]⁺ |
| 48 | 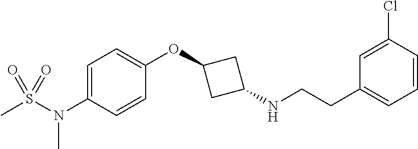 [SMILES: CN(c1ccc(O[C@H]2C[C@H](NCCc3cccc(Cl)c3)C2)cc1)S(C)(=O)=O] | trans-N-(4-(3-((3- chlorophenethyl)amino) cyclobutoxy)phenyl)-N- methylmethanesulfonamide | Calc'd 409.1 Found 409.0 |
| 49 | 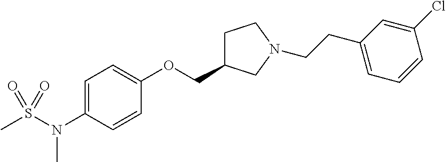 [SMILES: CN(c1ccc(OC[C@H]2CCN(CCc3cccc(Cl)c3)C2)cc1)S(C)(=O)=O] | (S)-N-(4-((1-(3- chlorophenethyl)pyrrolidin- 3-yl)methoxy)phenyl)-N- methylmethanesulfonamide | Calc'd 423.1 Found 423.0 |
| 50 | 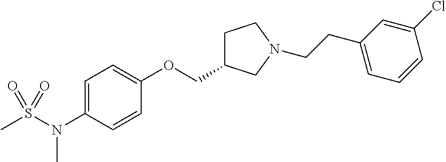 [SMILES: CN(c1ccc(OC[C@@H]2CCN(CCc3cccc(Cl)c3)C2)cc1)S(C)(=O)=O] | (R)-N-(4-((1-(3- chlorophenethyl)pyrrolidin- 3-yl)methoxy)phenyl)-N- methylmethanesulfonamide | Calc'd 423.1 Found 423.0 |
| 51 | 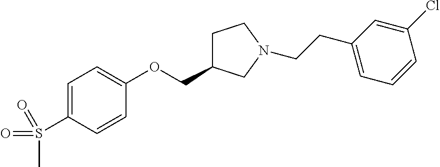 [SMILES: CS(=O)(=O)c1ccc(OC[C@H]2CCN(CCc3cccc(Cl)c3)C2)cc1] | (S)-1-(3-chlorophenethyl)-3- ((4- (methylsulfonyl)phenoxy) methyl)pyrrolidine | Calc'd 394.1 Found 394.1 |

### Example 14

### rac-trans-1-(3-chlorophenethyl)-3-methyl-4-((4-(methylsulfonyl)phenoxy)methyl)pyrrolidine (Compound 52)

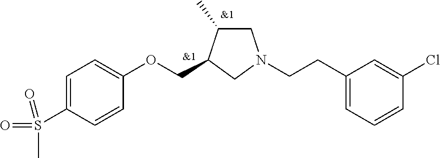 [SMILES: C[C@@H]1CN(CCc2cccc(Cl)c2)C[C@H]1COc1ccc(S(C)(=O)=O)cc1]

### Step 1: rac-trans-tert-butyl-3-methyl-4-((4-(methylsulfonyl)phenoxy)methyl)pyrrolidine-1-carboxylate

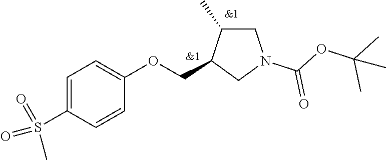 [SMILES: C[C@@H]1CN(C(=O)OC(C)(C)C)C[C@H]1COc1ccc(S(C)(=O)=O)cc1]

To a solution of 1-fluoro-4-methanesulfonylbenzene (0.405 g, 2.32 mmol) and rac-trans-tert-butyl-3-(hydroxymethyl)-4-methylpyrrolidine-1-carboxylate (1000 mg, 4.65 mmol) in DMF (4.6 mL) was added K₂CO₃ (1.28 g, 9.29 mmol). The reaction was heated to 110° C. for 16 h. The reaction mixture was cooled to room temperature and water (50 mL) was added. The mixture was extracted with EtOAc (3×50 mL). The combined organic layers were washed with brine (50 mL), dried over Na₂SO₄, filtered and concentrated in vacuo. The crude residue was purified by normal phase flash chromatography (Biotage 25 g cartridge, 0-10% MeOH in DCM) to give rac-trans-tert-butyl-3-(4-methanesulfonylphenoxymethyl)-4-methylpyrrolidine-1-carboxylate. MS=392.0 [M+Na]⁺.

### Step 2: rac-trans-3-methyl-4-((4-(methylsulfonyl)phenoxy)methyl)pyrrolidine

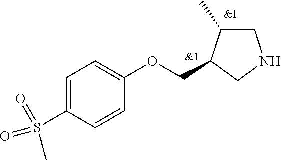 [SMILES: C[C@@H]1CNC[C@H]1COc1ccc(S(C)(=O)=O)cc1]

To a solution of rac-trans-tert-butyl-3-(4-methanesulfonylphenoxymethyl)-4-methylpyrrolidine-1-carboxylate (900 mg, 2.44 mmol) in MeOH (5 mL) was added HCl in dioxane (4 M, 5 mL). The reaction was allowed to stir for 2 h at room temperature. The reaction mixture was concentrated in vacuo to give rac-trans-3-(4-methanesulfonylphenoxymethyl)-4-methylpyrrolidine (HCl salt), which was taken to the next step without purification. MS=269.9 [M+H]⁺.

### Step 3: rac-trans-1-(3-chlorophenethyl)-3-methyl-4-((4 (methylsulfonyl)phenoxy)methyl)pyrrolidine (Compound 52)

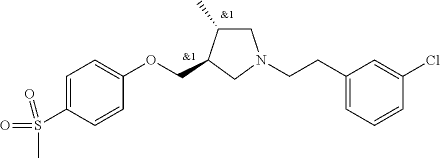 [SMILES: C[C@@H]1CN(CCc2cccc(Cl)c2)C[C@H]1COc1ccc(S(C)(=O)=O)cc1]

A mixture of rac-trans-3-(4-methanesulfonylphenoxymethyl)-4-methylpyrrolidine (100 mg, 0.327 mmol, HCl salt), 1-(2-bromoethyl)-3-chlorobenzene (0.086 g, 0.392 mmol), and K₂CO₃ (0.181 g, 1.308 mmol) was suspended in MeCN (1.6 mL). The resulting mixture was heated to 70° C. and allowed to stir for 18 h. The reaction was cooled to room temperature and concentrated in vacuo. Water (25 mL) was added, and the mixture was extracted with EtOAc (25 mL×3). The combined organic layers were washed with brine (25 mL), dried over Na₂SO₄, filtered and concentrated in vacuo. The crude residue was purified by reverse phase preparative HPLC (Phenomenex Kinetex C18 column, 5-50% MeCN/water with 0.1% formic acid modifier) to give rac-trans-1-(3-chlorophenethyl)-3-methyl-4-((4-(methylsulfonyl)phenoxy)methyl)pyrrolidine (Compound 52)¹H NMR (500 MHz, CD₃CN): δ 7.84 (d, J=8.9 Hz, 2H), 7.31-7.15 (m, 4H), 7.09 (d, J=8.9 Hz, 2H), 4.10-4.07 (m, 1H), 4.03-3.99 (m, 1H), 3.16-3.09 (m, 1H), 3.01 (s, 3H), 3.00-2.93 (m, 1H), 2.92-2.79 (m, 5H), 2.44-2.37 (m, 1H), 2.29-2.19 (m, 1H), 2.16-2.05 (m, 1H), 1.12 (d, J=6.7 Hz, 3H). MS=408.1 [M+H]⁺.

The following compounds in Table 18 were prepared according to procedures similar to steps 1-3 described for Compound 52 using the appropriate starting materials.

TABLE 18

| | | | Exact Mass |
|---|---|---|---|
| No. | Structure | IUPAC Name | [M + H]⁺ |
| 132 | 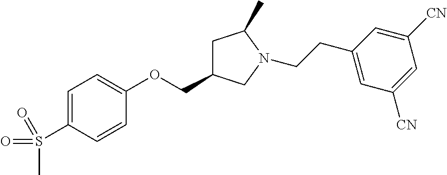 [SMILES: C[C@@H]1C[C@H](COc2ccc(S(C)(=O)=O)cc2)CN1CCc1cc(C#N)cc(C#N)c1] | 5-{2-[(2R,4S)-4-[(4- methanesulfonylphenoxy)me thyl]-2-methylpyrrolidin-1- yllethyl}benzene-1,3- dicarbonitrile | Calc'd 424.2 Found 424.1 |
| 133 | 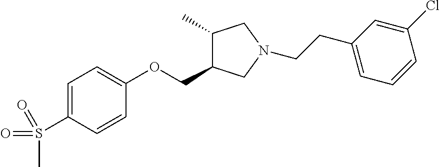 [SMILES: C[C@@H]1CN(CCc2cccc(Cl)c2)C[C@H]1COc1ccc(S(C)(=O)=O)cc1] | (35,4S)-1-[2-(3- chlorophenyl)ethyl]-3-[(4- methanesulfonylphenoxy) methyl]-4-methylpyrrolidine | Calc'd 408.1 Found 408.1 |
| 134 | 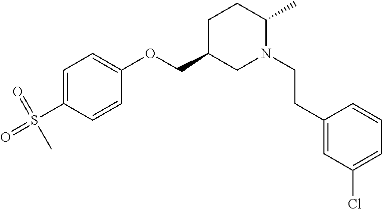 [SMILES: C[C@H]1CC[C@H](COc2ccc(S(C)(=O)=O)cc2)CN1CCc1cccc(Cl)c1] | (25,5S)-1-[2-(3- chlorophenyl)ethyl]-5-[(4- methanesulfonylphenoxy) methyl]-2-methylpiperidine | Calc'd 422.1 Found 422.2 |
| 160 | 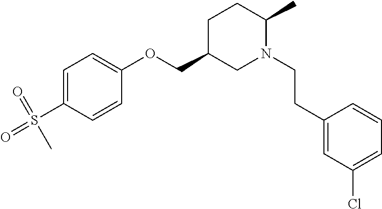 [SMILES: C[C@@H]1CC[C@H](COc2ccc(S(C)(=O)=O)cc2)CN1CCc1cccc(Cl)c1] | (2R,5S)-1-[2-(3- chlorophenyl)ethyl]-5-[(4- methanesulfonylphenoxy) methyl]-2-methylpiperidine | Calc'd 422.1 Found 422.2 |

### Example 15

### rac-trans or cis-1-(3-chlorophenethyl)-2-methyl-4-((4-(methylsulfonyl)phenoxy)methyl)pyrrolidine (Compound 53)

### rac-cis or trans-1-(3-chlorophenethyl)-2-methyl-4-((4-(methylsulfonyl)phenoxy)methyl)pyrrolidine (Compound 54)

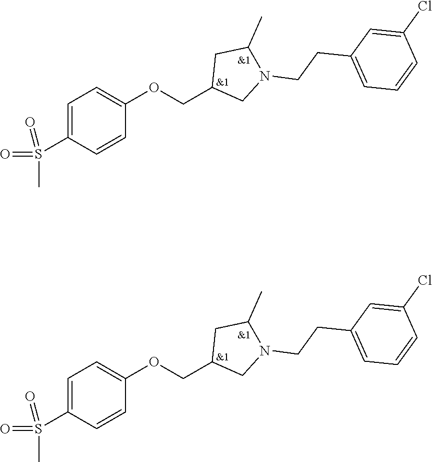 [SMILES: CC1CC(COc2ccc(S(C)(=O)=O)cc2)CN1CCc1cccc(Cl)c1 ; CC1CC(COc2ccc(S(C)(=O)=O)cc2)CN1CCc1cccc(Cl)c1]

### Step 1: tert-butyl 4-(hydroxymethyl)-2-methylpyrrolidine-1-carboxylate

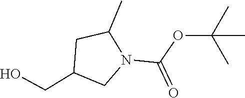

To a 0° C. solution of 1-(tert-butoxycarbonyl)-5-methylpyrrolidine-3-carboxylic acid (450 mg, 1.96 mmol) in THF (10 mL) was added BH₃·THF (1 M, 3.14 mL). The mixture was warmed to room temperature and stirred for 2 h. The reaction mixture was cooled to 0° C., quenched with aqueous 2 M HCl (20 mL), and stirred at room temperature for 20 min. The mixture was extracted with EtOAc (20 mL×3). The combined organic layers were washed with saturated aqueous NaHCO₃ (40 mL), brine (40 mL), dried over Na₂SO₄, filtered and concentrated in vacuo to give tert-butyl 4-(hydroxymethyl)-2-methylpyrrolidine-1-carboxylate, which was used in the next step without further purification. MS=160.1 [M-C₄H₇+H]⁺.

### Step 2: 1-(3-chlorophenethyl)-2,2-dimethyl-4-((4-(methylsulfonyl)phenoxy)methyl) pyrrolidine

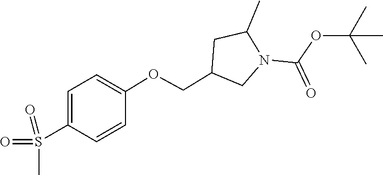 [SMILES: CC1CC(COc2ccc(S(C)(=O)=O)cc2)CN1C(=O)OC(C)(C)C]

A mixture of tert-butyl 4-(hydroxymethyl)-2-methylpyrrolidine-1-carboxylate (380 mg, 1.77 mmol), 1-fluoro-4-(methylsulfonyl)benzene (399 mg, 2.29 mmol) and Cs₂CO₃ (1.73 g, 5.30 mmol) in DMF (6 mL) was stirred at 100° C. for 16 h. After cooling to room temperature, the mixture was poured into water (30 mL) at 0° C. and extracted with EtOAc (30 mL×3). The combined organic layers were washed with water (60 mL), brine (60 mL), dried over Na₂SO₄, filtered and concentrated in vacuo. The crude residue was purified by normal phase silica gel chromatography (Biotage 12 g cartridge, 25-50% EtOAc in petroleum ether) to give tert-butyl 2-methyl-4-((4-(methylsulfonyl)phenoxy)methyl)pyrrolidine-1-carboxylate. MS=314.1 [M+Na]⁺.

### Step 3: 2-methyl-4-[(4-methylsulfonylphenoxy) methyl]pyrrolidine

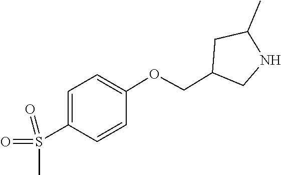 [SMILES: CC1CC(COc2ccc(S(C)(=O)=O)cc2)CN1]

To a 0° C. solution of tert-butyl 2-methyl-4-((4-(methylsulfonyl)phenoxy)methyl)pyrrolidine-1-carboxylate (570 mg, 1.54 mmol) in DCM (5 mL) was added TFA (2.5 mL, 33.7 mmol). The mixture was warmed to room temperature and stirred for 1 h, then was concentrated under reduced pressure. The residue was dissolved in aqueous NaHCO₃ (15 mL) and was extracted with a solution of 1:3 isopropanol:DCM (15 mL×3). The combined organic layers were dried over Na₂SO₄, filtered and the filtrate was concentrated in vacuo to give 2-methyl-4-[(4-methylsulfonylphenoxy) methyl]pyrrolidine, which was used in the next step without further purification. MS=270.1 [M+H]⁺.

### Step 4: rac-trans or cis-1-(3-chlorophenethyl)-2-methyl-4-((4-(methylsulfonyl)phenoxy)methyl)pyrrolidine (Compound 53); rac-cis or trans-1-(3-chlorophenethyl)-2-methyl-4-((4-(methylsulfonyl)phenoxy)methyl)pyrrolidine (Compound 54)

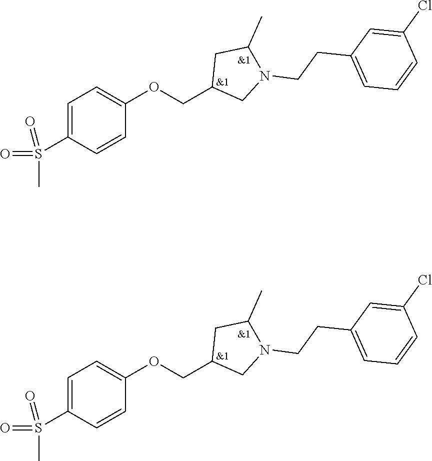 [SMILES: CC1CC(COc2ccc(S(C)(=O)=O)cc2)CN1CCc1cccc(Cl)c1 ; CC1CC(COc2ccc(S(C)(=O)=O)cc2)CN1CCc1cccc(Cl)c1]

A mixture of 2-methyl-4-((4-(methylsulfonyl)phenoxy)methyl)pyrrolidine (350 mg, 1.30 mmol) and 2-(3-chlorophenyl)acetaldehyde (261 mg, 1.69 mmol) in MeOH (4 mL) was stirred at room temperature for 10 min. After cooling to 0° C., NaBH₃CN (122 mg, 1.95 mmol) was added, and the mixture was stirred at room temperature for 1 h. The mixture was quenched by the addition of water (0.3 mL) and purified by reverse phase preparative HPLC (Waters Xbridge C18 column, 45-65% MeCN/10 mM NH₄HCO₃ in water). The first eluting enantiomeric mixture of the title compound, Compound 53: ¹H NMR (400 MHz, DMSO-d₆): δ 7.83 (d, J=8.4 Hz, 2H), 7.33-7.20 (m, 4H), 7.16-7.14 (m, 2H), 4.0-3.94 (m, 2H), 3.35-3.33 (m, 1H), 3.15 (s, 3H), 2.97-2.95 (m, 1H), 2.78-2.71 (m, 2H), 2.57-2.56 (m, 1H), 2.46-2.45 (m, 1H), 2.29-2.28 (m, 1H), 2.06-2.01 (m, 1H), 1.73-1.71 (m, 1H), 1.54-1.53 (m, 1H), 1.01 (d, J=6.0 Hz, 3H). MS=408.2 [M+H]⁺. The second eluting enantiomeric mixture of the title compound, Compound 54: ¹H NMR (400 MHz, DMSO-d₆): δ 7.83 (d, J=8.8 Hz, 2H), 7.31 (s, 1H), 7.25-7.14 (m, 5H), 3.96-3.89 (m, 2H), 3.16 (s, 3H), 3.08-3.06 (m, 1H), 2.94-2.93 (m, 1H), 2.76-2.68 (m, 2H), 2.48-2.47 (m, 1H), 2.34-2.29 (m, 3H), 2.13-2.09 (m, 1H), 1.09-1.00 (m, 4H). MS=408.2 [M+H]⁺.

### Example 16

### (2R,4S) or (2R,4R) or (2S,4R) or (2S,4S)-1-(3-chlorophenethyl)-2-methyl-4-((4-(methylsulfonyl)phenoxy)methyl)pyrrolidine (Compound 55)

### (2R,4R) or (2S,4R) or (2S,4S) or (2R,4S)-1-(3-chlorophenethyl)-2-methyl-4-((4-(methylsulfonyl)phenoxy)methyl)pyrrolidine (Compound 56)

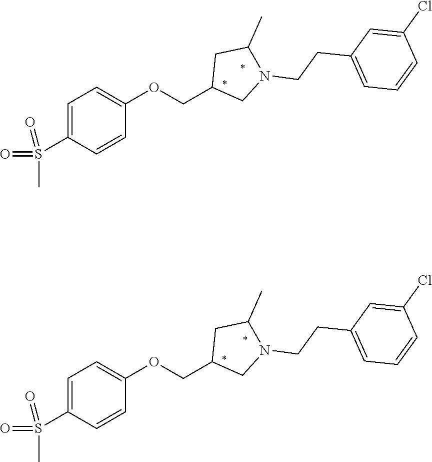 [SMILES: CC1CC(COc2ccc(S(C)(=O)=O)cc2)CN1CCc1cccc(Cl)c1 ; CC1CC(COc2ccc(S(C)(=O)=O)cc2)CN1CCc1cccc(Cl)c1]

rac-trans or cis-1-(3-chlorophenethyl)-2-methyl-4-((4-(methylsulfonyl)phenoxy)methyl)pyrrolidine (Compound 53, 230 mg, 0.563 mmol) was separated by preparative chiral SFC (DAICEL CHIRALPAK AD-3, 44% MeOH with 0.1% NH₄OH in CO₂). The first eluting enantiomer of the title compound, Compound 55: ¹H NMR (400 MHz, DMSO-d₆): δ 7.83 (d, J=8.8 Hz, 2H), 7.33-7.21 (m, 4H), 7.17-7.14 (m, 2H), 4.04-3.94 (m, 2H), 3.32-3.31 (m, 1H), 3.15 (s, 3H), 2.98-2.96 (m, 1H), 2.78-2.70 (m, 2H), 2.56-2.55 (m, 1H), 2.44-2.43 (m, 1H), 2.27-2.26 (m, 1H), 2.03-2.02 (m, 1H), 1.76-1.72 (m, 1H), 1.57-1.49 (m, 1H), 1.01 (d, J=4.8 Hz, 3H). MS=408.2 [M+H]⁺. The second eluting enantiomer of the title compound, Compound 56: ¹H NMR (400 MHz, DMSO-d₆): δ 7.83 (d, J=8.8 Hz, 2H), 7.33-7.22 (m, 4H), 7.17-7.14 (m, 2H), 4.04-3.93 (m, 2H), 3.36-3.35 (m, 1H), 3.15 (s, 3H), 2.98-2.97 (m, 1H), 2.78-2.70 (m, 2H), 2.57-2.56 (m, 1H), 2.47-2.46 (m, 1H), 2.33-2.28 (m, 1H), 2.07-2.04 (m, 1H), 1.73-1.72 (m, 1H), 1.54-1.51 (m, 1H), 1.01 (d, J=6.0 Hz, 3H). MS=408.2 [M+H]⁺.

### Example 17

### (2S,4R) or (2S,4S) or (2R,4S) or (2R,4R)-1-(3-chlorophenethyl)-2-methyl-4-((4-(methylsulfonyl)phenoxy)methyl)pyrrolidine (Compound 57)

### (2S,4S) or (2R,4S) or (2R,4R) or (2S,4R)-1-(3-chlorophenethyl)-2-methyl-4-((4-(methylsulfonyl)phenoxy)methyl)pyrrolidine (Compound 58)

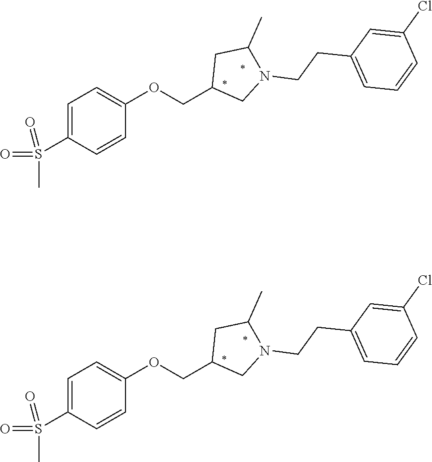 [SMILES: CC1CC(COc2ccc(S(C)(=O)=O)cc2)CN1CCc1cccc(Cl)c1 ; CC1CC(COc2ccc(S(C)(=O)=O)cc2)CN1CCc1cccc(Cl)c1]

rac-cis or trans-1-(3-chlorophenethyl)-2-methyl-4-((4-(methylsulfonyl)phenoxy)methyl)pyrrolidine (Compound 54, 160 mg, 0.392 μmol) was separated by preparative chiral SFC (DAICEL CHIRALPAK AD-3, 33% MeOH with 0.1% NH₄OH in CO₂). The first eluting enantiomer of the title compound, Compound 57: ¹H NMR (400 MHz, DMSO-d₆): δ 7.83 (d, J=8.8 Hz, 2H), 7.31 (s, 1H), 7.23-7.14 (m, 5H), 3.95-3.88 (m, 2H), 3.15 (s, 3H), 3.06-3.05 (m, 1H), 2.94-2.93 (m, 1H), 2.71-2.67 (m, 2H), 2.53-2.52 (m, 1H), 2.33-2.25 (m, 3H), 2.14-2.13 (m, 1H), 1.11-0.96 (m, 4H). MS=408.2 [M+H]⁺. The second eluting enantiomer of the title compound, Compound 58: ¹H NMR (400 MHz, DMSO-d₆): δ 7.83 (d, J=8.8 Hz, 2H), 7.31 (s, 1H), 7.27-7.14 (m, 5H), 3.95-3.91 (m, 2H), 3.15 (s, 3H), 3.08-3.05 (m, 1H), 2.98-2.89 (m, 1H), 2.76-2.67 (m, 2H), 2.53-2.52 (m, 1H), 2.33-2.28 (m, 3H), 2.2-2.11 (m, 1H), 1.09-0.99 (m, 1H), 0.96-0.85 (m, 3H). MS=408.2 [M+H]⁺.

The following compounds in Table 19 were prepared according to procedures similar to those described for Example 15 to 17 using the appropriate starting materials.

TABLE 19

| | | | Exact | Chiral | Chiral |
|---|---|---|---|---|---|
| | | | Mass | purification | Elution |
| No. | Structure | IUPAC Name | [M + H]⁺ | column | order |
| 135 | 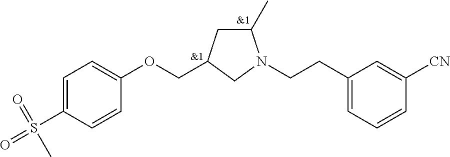 [SMILES: CC1CC(COc2ccc(S(C)(=O)=O)cc2)CN1CCc1cccc(C#N)c1] | rac-cis or trans- 3-{2-[4-[(4- methanesulfon- ylphenoxy)meth- yl]-2-methyl- pyrrolidin-1-yl]- ethyl}benzo-nitrile | Calc'd 399.2 Found 399.1 | Prep- HPLC Waters Xbridge BEH C18 | 1st |
| 136 | 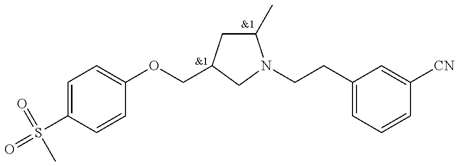 [SMILES: CC1CC(COc2ccc(S(C)(=O)=O)cc2)CN1CCc1cccc(C#N)c1] | rac-trans or cis- 3-{2-[4-[(4- methanesulfon- ylphenoxy)meth- yl]-2-methyl- pyrrolidin-1-yl]- ethyl}benzo- nitrile | Calc'd 399.2 Found 399.1 | Prep- HPLC Waters Xbridge BEH C18 | 2nd |
| 137 | 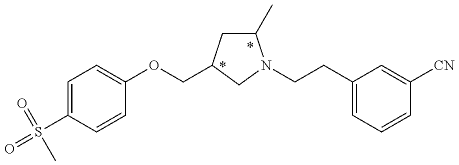 [SMILES: CC1CC(COc2ccc(S(C)(=O)=O)cc2)CN1CCc1cccc(C#N)c1] | 3-{2-[(2S,4R or 2R,4S or 2R,4R or 2S,4S)-4-[(4- methanesulfon- ylphenoxy)meth- yl]-2-methyl- pyrrolidin-1-yl]- ethyl}benzo- nitrile | Calc'd 399.2 Found 399.1 | Chiralpak AD | 1st |
| 138 | 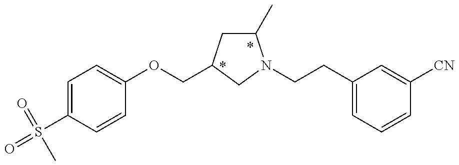 [SMILES: CC1CC(COc2ccc(S(C)(=O)=O)cc2)CN1CCc1cccc(C#N)c1] | 3-{2-[(2R,4S or 2S,4R or 2R,4R or 2S,4S)-4-[(4- methanesulfon- ylphenoxy)meth-yl]-2-methyl- pyrrolidin-1-yl]- ethyl}benzo- nitrile | Calc'd 399.2 Found 399.1 | Chiralpak AD | 2nd |
| 139 | 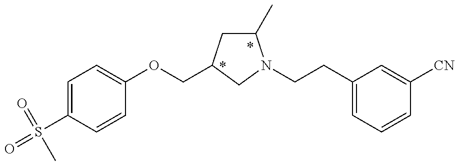 [SMILES: CC1CC(COc2ccc(S(C)(=O)=O)cc2)CN1CCc1cccc(C#N)c1] | 3-{2-[(2S,4R or 2R,4S or 2S,4S or 2R,4R)-4-[(4- methanesulfon- ylphenoxy)meth- yl]-2-methyl- pyrrolidin-1-yl]- ethyl}benzo- nitrile | Calc'd 399.2 Found 399.1 | Chiralpak AD | 1st |
| 140 | 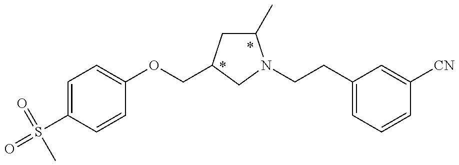 [SMILES: CC1CC(COc2ccc(S(C)(=O)=O)cc2)CN1CCc1cccc(C#N)c1] | 3-{2-[(2S,4R or 2R,4S or 2R,4R or 2S,4S)-4-[(4- methanesulfon- ylphenoxy)meth- yl]-2-methyl- pyrrolidin-1-yl]- ethyl}benzo- nitrile | Calc'd 399.2 Found 399.1 | Chiralpak AD | 2nd |
| 141 | 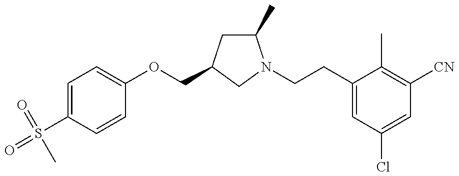 [SMILES: Cc1c(C#N)cc(Cl)cc1CCN1C[C@@H](COc2ccc(S(C)(=O)=O)cc2)C[C@H]1C] | 5-chloro-3-{2- [(2R,4S)-4-[(4- methanesulfon- ylphenoxy)meth- yl]-2-methyl- pyrrolidin-1-yl]- ethyl}-2-methyl- benzonitrile | Calc'd 447.1 Found 447.2 | n/a | n/a |
| 142 | 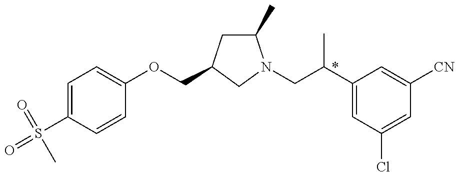 [SMILES: CC(CN1C[C@@H](COc2ccc(S(C)(=O)=O)cc2)C[C@H]1C)c1cc(Cl)cc(C#N)c1] | 3-chloro-5-[(2R or 2S)-1-[(2R, 4S)-4-[(4-meth- anesulfonyl- phenoxy)meth- yl]-2-methyl- pyrrolidin-1-yl]- propan-2-yl]- benzonitrile | Calc'd 447.1 Found 447.2 | prep- HPLC Waters Xbridge BEH C18 | 2nd |
| 143 | 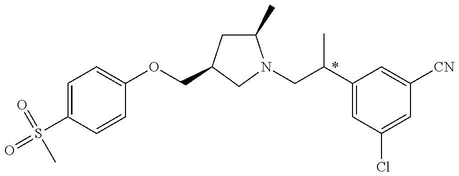 [SMILES: CC(CN1C[C@@H](COc2ccc(S(C)(=O)=O)cc2)C[C@H]1C)c1cc(Cl)cc(C#N)c1] | 3-chloro-5-[(2S or 2R)-1-[(2R, 4S)-4-[(4-meth- anesulfonyl- phenoxy)meth- yl]-2-methyl- pyrrolidin-1-yl]- propan-2-yl]- benzonitrile | Calc'd 447.1 Found 447.2 | prep- HPLC Waters Xbridge BEH C18 | 1st |
| 144 | 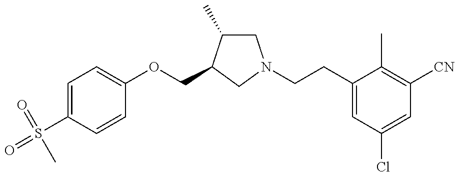 [SMILES: Cc1c(C#N)cc(Cl)cc1CCN1C[C@@H](COc2ccc(S(C)(=O)=O)cc2)[C@H](C)C1] | 5-chloro-3-{2- [(3S,4S)-3-[(4- methanesulfon- ylphenoxy)meth- yl]-4-methyl- pyrrolidin-1-yl]- ethyl}-2-methyl- benzonitrile | Calc'd 447.1 Found 447.2 | n/a | n/a |
| 145 | 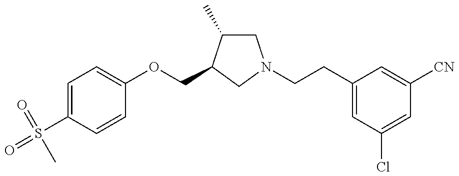 [SMILES: C[C@@H]1CN(CCc2cc(Cl)cc(C#N)c2)C[C@H]1COc1ccc(S(C)(=O)=O)cc1] | 5-{2-[(3S,4S)-3- [(4-methane- sulfonylphen- oxy)methyl]-4- methylpyrroli- din-1-yl]ethyl}- benzene-1,3- dicarbonitrile | Calc'd 424.2 Found 424.1 | n/a | n/a |
| 146 | 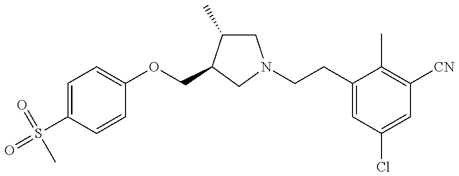 [SMILES: Cc1c(C#N)cc(Cl)cc1CCN1C[C@@H](COc2ccc(S(C)(=O)=O)cc2)[C@H](C)C1] | 3-chloro-5-[1- [(3S,4S)-3-[(4- methanesulfon- ylphenoxy)meth- yl]-4-methyl- pyrrolidin-1-yl]- propan-2-yl]- benzonitrile | Calc'd 447.1 Found 447.1 | n/a | n/a |
| 147 | 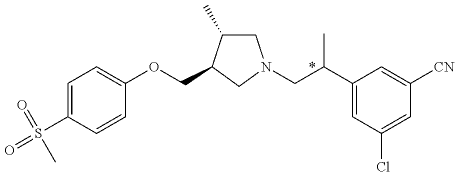 [SMILES: CC(CN1C[C@@H](COc2ccc(S(C)(=O)=O)cc2)[C@H](C)C1)c1cc(Cl)cc(C#N)c1] | 3-chloro-5-[(2R or 2S)-1-[(3S, 4S)-3-[(4-meth- anesulfonylphen- oxy)methyl]-4- methylpyrroli- din-1-yl]propan- 2-yl]benzonitrile | Calc'd 447.1 Found 447.1 | Chiralpk AD | 1st |
| 148 | 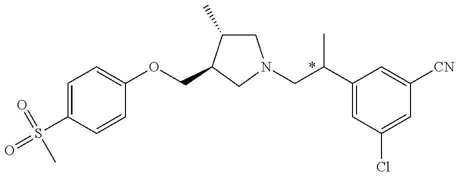 [SMILES: CC(CN1C[C@@H](COc2ccc(S(C)(=O)=O)cc2)[C@H](C)C1)c1cc(Cl)cc(C#N)c1] | 3-chloro-5-[(2S or 2R)-1-[(3S, 4S)-3-[(4-meth- anesulfonylphen- oxy)methyl]-4- methylpyrroli- din-1-yl]propan- 2-yl]benzonitrile | Calc'd 447.1 Found 447.1 | Chiralpk AD | 2nd |
| 149 | 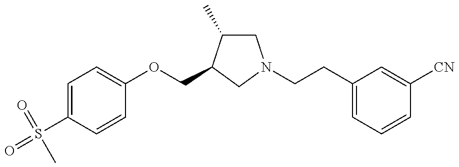 [SMILES: C[C@@H]1CN(CCc2cccc(C#N)c2)C[C@H]1COc1ccc(S(C)(=O)=O)cc1] | 3-{2-[(3S,4S)- 3-[(4-methane- sulfonylphen- oxy)methyl]-4- methylpyrroli- din-1-yl]ethyl}- benzonitrile | Calc'd 399.2 Found 399.1 | n/a | n/a |
| 150 | 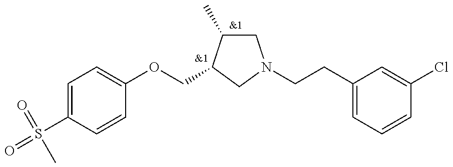 [SMILES: C[C@@H]1CN(CCc2cccc(Cl)c2)C[C@@H]1COc1ccc(S(C)(=O)=O)cc1] | rac-cis-1-[2-(3- chlorophenyl)- ethyl]-3-[(4- methanesulfon- ylphenoxy)meth- yl]-4-methyl- pyrrolidine | Calc'd 408.1 Found 408.3 | n/a | n/a |
| 151 | 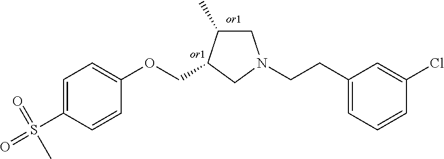 [SMILES: C[C@@H]1CN(CCc2cccc(Cl)c2)C[C@@H]1COc1ccc(S(C)(=O)=O)cc1] | (3R,4S or 3S, 4R)-1-[2-(3- chlorophenyl)- ethyl]-3-[(4- methanesulfon- ylphenoxy)meth- yl]-4-methyl- pyrrolidine | Calc'd 408.1 Found 408.3 | Chiralcel OJ | 1st |
| 152 | 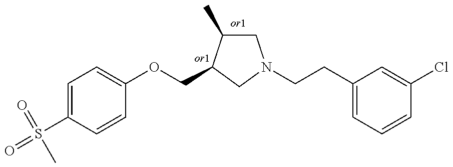 [SMILES: C[C@H]1CN(CCc2cccc(Cl)c2)C[C@H]1COc1ccc(S(C)(=O)=O)cc1] | (3S,4R or 3R, 4S)-1-[2-(3- chlorophenyl)- ethyl]-3-[(4- methanesulfon- ylphenoxy)meth- yl]-4-methyl- pyrrolidine | Calc'd 408.1 Found 408.2 | Chiralcel OJ | 2nd |
| 153 | 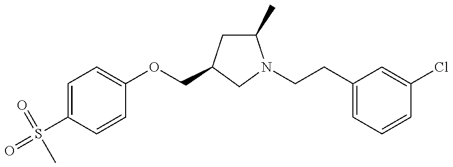 [SMILES: C[C@@H]1C[C@H](COc2ccc(S(C)(=O)=O)cc2)CN1CCc1cccc(Cl)c1] | (2R,4S)-1-[2-(3- chlorophenyl)- ethyl]-4-[(4- methanesulfon- ylphenoxy)meth- yl]-2-methyl- pyrrolidine | Calc'd 408.1 Found 408.1 | n/a | n/a |
| 154 | 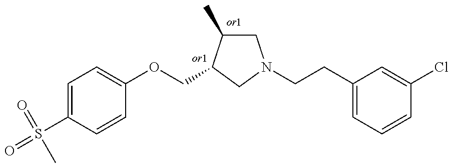 [SMILES: C[C@H]1CN(CCc2cccc(Cl)c2)C[C@@H]1COc1ccc(S(C)(=O)=O)cc1] | (3R,4R or 3S, 4S)-1-[2-(3- chlorophenyl)- ethyl]-3-[(4- methanesulfon- ylphenoxy)meth- yl]-4-methyl- pyrrolidine | Calc'd 408.1 Found 408.1 | Chiralcel OD | 1st |
| 155 | 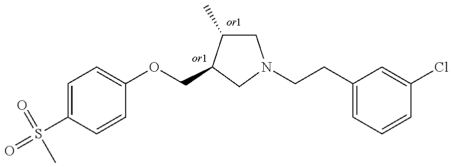 [SMILES: C[C@@H]1CN(CCc2cccc(Cl)c2)C[C@H]1COc1ccc(S(C)(=O)=O)cc1] | (35,4S or 3R, 4R)-1-[2-(3- chlorophenyl)- ethyl]-3-[(4- methanesulfon- ylphenoxy)meth- yl]-4-methyl- pyrrolidine | Calc'd 408.1 Found 408.1 | Chiralcel OD | 2nd |

### Example 18

### (S)—N-(4-(3-((3-chlorophenethyl)(cyclopropylmethyl)amino)-2-hydroxypropoxy)phenyl)-N-methylmethanesulfonamide (Compound 59)

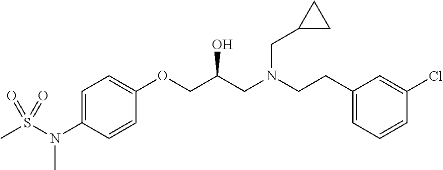 [SMILES: CN(c1ccc(OC[C@@H](O)CN(CCc2cccc(Cl)c2)CC2CC2)cc1)S(C)(=O)=O]

A solution of (S)—N-(4-(3-((3-chlorophenethyl)amino)-2-hydroxypropoxy)phenyl)-N-methylmethanesulfonamide (Compound 86, 30.0 mg, 0.073 mmol), (bromomethyl)cyclopropane (21.1 μL, 0.218 mmol), and K₂CO₃ (40 mg, 0.291 mmol) in MeCN (0.73 mL) was heated at 85° C. for 18 h. After cooling to room temperature, solids were removed via filtration, and the filtrate was concentrated in vacuo. The crude residue was purified by reverse phase preparative HPLC (Phenomenex Kinetex C18 column, 10-70% MeCN/water with 0.1% formic acid modifier) to give (S)—N-(4-(3-((3-chlorophenethyl)(cyclopropylmethyl)amino)-2-hydroxypropoxy)phenyl)-N-methylmethanesulfonamide (Compound 59). ¹H NMR (500 MHz, DMSO-d₆, 29/31 H): δ 7.25-7.13 (m, 4H), 7.10-7.05 (m, 2H), 6.84-6.74 (m, 2H), 3.86-3.73 (m, 2H), 3.70-3.67 (m, 1H), 3.08 (s, 3H), 2.79 (s, 3H), 2.77-2.56 (m, 4H), 2.51-2.40 (m, 1H), 2.35-2.33 (m, 2H), 0.79-0.68 (m, 1H), 0.39-0.27 (m, 2H), 0.06-0.07 (m, 2H). MS=467.1 [M+H]⁺.

The following compound in Table 20 was prepared according to procedures similar to those described for Compound 59 using the appropriate starting materials.

TABLE 20

| | Starting | | | Exact Mass |
|---|---|---|---|---|
| No. | Material | Structure | IUPAC Name | [M + H]⁺ |
| 60 | Compound 86 | 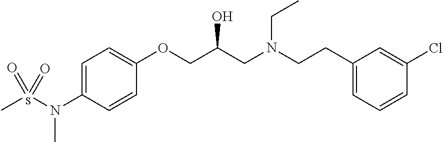 [SMILES: CCN(CCc1cccc(Cl)c1)C[C@H](O)COc1ccc(N(C)S(C)(=O)=O)cc1] | (S)-N-(4-(3-((3- chlorophenethyl)(ethyl) amino)-2- hydroxypropoxy)phenyl)-N- methylmethanesulfonamide | Calc'd 441.2 Found 441.2 |

### Example 19

### (R)—N-(4-(3-((3-chlorophenethyl)amino)-2-methylpropoxy)phenyl)-N-methylmethanesulfonamide (Compound 61)

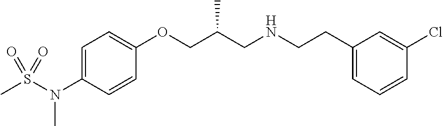 [SMILES: C[C@H](CNCCc1cccc(Cl)c1)COc1ccc(N(C)S(C)(=O)=O)cc1]

### Step 1: (S)—N-(4-(3-bromo-2-methylpropoxy)phenyl)-N-methylmethanesulfonamide

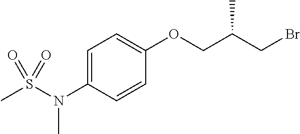 [SMILES: C[C@H](CBr)COc1ccc(N(C)S(C)(=O)=O)cc1]

A solution of N-(4-hydroxyphenyl)-N-methylmethanesulfonamide (124 mg, 0.616 mmol), (2S)-3-bromo-2-methylpropan-1-ol (123 mg, 0.801 mmol), and polymer-bound PPh₃ (0.411 g, 3 mmol/g, 1.23 mmol) in toluene (2 mL) was stirred at room temperature for 20 min, and then cooled to 0° C. Diisopropylazodioxcarboxylate (153 μL, 0.77 mmol) was added dropwise, the mixture was warmed to room temperature and then stirred for 12 h. Solids were removed by filtration and washed with DCM, and the filtrate was concentrated in vacuo. The crude residue was purified by normal phase silica gel chromatography (Biotage 10 g cartridge, 0-60% EtOAc in hexanes) to give (S)—N-(4-(3-bromo-2-methylpropoxy)phenyl)-N-methylmethanesulfonamide. MS=338.9 [M+H]⁺.

### Step 2: (R)—N-(4-(3-((3-chlorophenethyl)amino)-2-methylpropoxy)phenyl)-N-methylmethanesulfonamide (Compound 61)

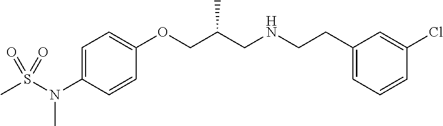 [SMILES: C[C@H](CNCCc1cccc(Cl)c1)COc1ccc(N(C)S(C)(=O)=O)cc1]

A solution of N-(4-((2S)-3-bromo-2-methylpropoxy)phenyl)-N-methylmethanesulfonamide (155 mg, 0.461 mmol), 2-(3-chlorophenyl)ethanamine (192 μL, 1.38 mmol), and K₂CO₃ (191 mg, 1.38 mmol) in MeCN was stirred at 80° C. for 18 h. After cooling to room temperature, solids were removed by filtration and washed with DCM, and the filtrate was concentrated in vacuo. The residue was taken up in DCM (3 mL) and washed with water (3 mL). The organic layer was dried over Na₂SO₄, filtered and the filtrate was concentrated in vacuo. The crude residue was purified by reverse phase preparative HPLC (Phenomenex Kinetex C18 column, 0-70% MeCN/water with 0.1% formic acid modifier). A second purification was performed by normal phase silica gel chromatography (Biotage 10 g cartridge, 0-20% MeOH in DCM) to give (R)—N-(4-(3-((3-chlorophenethyl)amino)-2-methylpropoxy)phenyl)-N-methylmethanesulfonamide (Compound 61). ¹H NMR (500 MHz, CD₃CN): δ 8.45-8.10 (m, 1H), 7.51-7.17 (m, 6H), 6.98-6.80 (m, 2H), 3.92 (d, J=6.1 Hz, 2H), 3.24 (s, 3H), 2.98 (d, J=8.8 Hz, 2H), 2.85 (s, 6H), 2.79-2.65 (m, 1H), 2.25-2.18 (m, 1H), 1.03 (dd, J=6.8, 2.4 Hz, 3H). MS=411.2 [M+H]⁺.

### Example 20

### (2S)-1-(2-(3-chlorobenzyl)piperidin-1-yl)-3-(4-(methylsulfonyl)phenoxy)propan-2-ol (Compound 62)

### (2S)-1-((S) or (R)-2-(3-chlorobenzyl)piperidin-1-yl)-3-(4-(methylsulfonyl)phenoxy)propan-2-ol (Compound 63)

### (2S)-1-((R) or (S)-2-(3-chlorobenzyl)piperidin-1-yl)-3-(4-(methylsulfonyl)phenoxy)propan-2-ol (Compound 64)

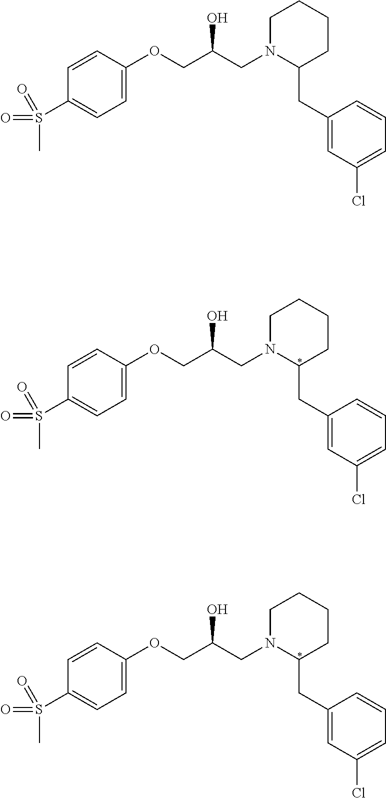 [SMILES: CS(=O)(=O)c1ccc(OC[C@@H](O)CN2CCCCC2Cc2cccc(Cl)c2)cc1 ; CS(=O)(=O)c1ccc(OC[C@@H](O)CN2CCCCC2Cc2cccc(Cl)c2)cc1 ; CS(=O)(=O)c1ccc(OC[C@@H](O)CN2CCCCC2Cc2cccc(Cl)c2)cc1]

### Step 1: (2S)-1-(2-(3-chlorobenzyl)piperidin-1-yl)-3-(4-(methylsulfonyl)phenoxy)propan-2-ol (Compound 62)

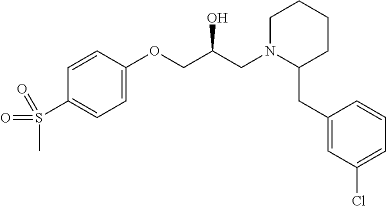 [SMILES: CS(=O)(=O)c1ccc(OC[C@@H](O)CN2CCCCC2Cc2cccc(Cl)c2)cc1]

To a solution of (S)-2-((4-(methylsulfonyl)phenoxy)methyl)oxirane (Intermediate C, 50 mg, 0.22 mmol) in EtOH (1.5 mL) was added 2-[(3-chlorophenyl)methyl]piperidine (55 mg, 0.25 mmol). The mixture was heated to 80° C. and allowed to stir for 18 h. After cooling the reaction to room temperature, the mixture was concentrated in vacuo. The crude residue was purified by reverse phase preparative HPLC (Phenomenex Kinetex C18 column, 0-40% MeCN/water with 0.1% formic acid modifier) to give (2S)-1-(2-(3-chlorobenzyl)piperidin-1-yl)-3-(4-(methylsulfonyl)phenoxy)propan-2-ol (Compound 62). ¹H NMR (500 MHz, DMSO-d₆): δ 8.15 (s, 1H), 7.86-7.77 (m, 2H), 7.32-7.24 (m, 2H), 7.23-7.19 (m, 1H), 7.17-7.13 (m, 3H), 4.16-4.00 (m, 1H), 4.01-3.83 (m, 2H), 3.15 (s, 3H), 3.04-2.91 (m, 1H), 2.89-2.76 (m, 2H), 2.73-2.70 (m, 1H), 2.62-2.54 (m, 2H), 2.46-2.35 (m, 1H), 1.62-1.57 (m, 1H), 1.54-1.35 (m, 3H), 1.35-1.24 (m, 1H), 1.24-1.05 (m, 1H). MS=437.9 [M+H]⁺.

### Step 2: (S)-1-((S) or (R)-2-(3-chlorobenzyl)piperidin-1-yl)-3-(4-(methylsulfonyl)phenoxy)propan-2-ol (Compound 63); (S)-1-((R) or (S)-2-(3-chlorobenzyl)piperidin-1-yl)-3-(4-(methylsulfonyl)phenoxy)propan-2-ol (Compound 64)

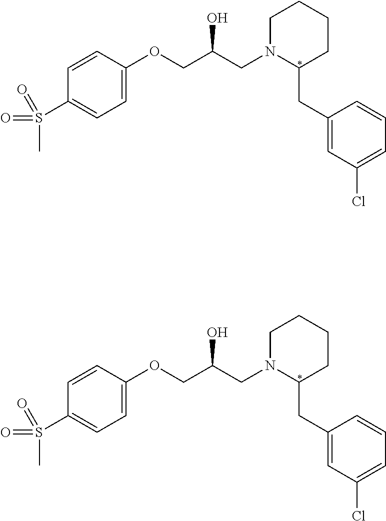 [SMILES: CS(=O)(=O)c1ccc(OC[C@@H](O)CN2CCCCC2Cc2cccc(Cl)c2)cc1 ; CS(=O)(=O)c1ccc(OC[C@@H](O)CN2CCCCC2Cc2cccc(Cl)c2)cc1]

(2S)-1-(2-(3-chlorobenzyl)piperidin-1-yl)-3-(4-(methylsulfonyl)phenoxy)propan-2-ol was separated by preparative chiral SFC (ChromegaChiral CC5 column, 45% MeOH with 0.25% isopropylamine in CO₂). The first eluting diastereomer of the title compound, Compound 63: ¹H NMR (500 MHz, CD₃CN): δ 7.91-7.79 (m, 2H), 7.31-7.27 (m, 2H), 7.23 (d, J=8.0 Hz, 1H), 7.18 (d, J=7.6 Hz, 1H), 7.13 (d, J=8.8 Hz, 2H), 4.69 (br. s, 1H), 4.11-4.08 (m, 1H), 4.03-3.98 (m, 2H), 3.04-3.00 (m, 4H), 2.98-2.87 (m, 3H), 2.72 (app dd, J=13.2, 9.3 Hz, 1H), 2.67-2.60 (m, 2H), 1.75-1.65 (m, 1H), 1.62-1.56 (m, 3H), 1.47-1.41 (m, 1H), 1.41-1.33 (m, 1H). MS=437.9 [M+H]⁺. The second eluting diastereomer of the title compound, Compound 64: ¹H NMR (500 MHz, CD₃CN): δ 7.86 (d, J=8.6 Hz, 2H), 7.33-7.26 (m, 2H), 7.22 (d, J=8.1 Hz, 1H), 7.18 (d, J=7.6 Hz, 1H), 7.12 (d, J=8.5 Hz, 2H), 4.69 (br s, 1H), 4.09 (app d, J=7.2 Hz, 1H), 4.01-3.98 (m, 2H), 3.12-3.00 (m, 5H), 2.86-2.82 (m, 2H), 2.72 (app dd, J=12.6, 4.7 Hz, 1H), 2.65 (app dd, J=13.4, 8.8 Hz, 1H), 2.54-2.49 (m, 1H), 1.71-1.67 (m, 1H), 1.63-1.53 (m, 3H), 1.43-1.38 (m, 1H), 1.36-1.32 (m, 1H). MS=438.0 [M+H].⁺

The following compounds in Table 21 were prepared according to procedures similar to those described for Compounds 63-64 using the appropriate starting materials.

TABLE 21

| | Inter- | | | Exact | | Chiral |
|---|---|---|---|---|---|---|
| | me- | | | Mass | Chiral | Elution |
| No. | diate | Structure | IUPAC Name | [M + H]⁺ | Column | Order |
| 65 | A | 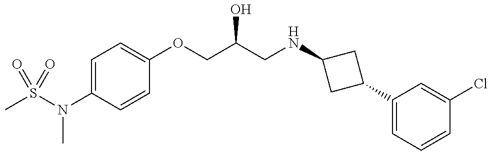 [SMILES: CN(c1ccc(OC[C@@H](O)CN[C@H]2C[C@H](c3cccc(Cl)c3)C2)cc1)S(C)(=O)=O] | N-(4-((S)-3- ((trans-3-(3- chlorophenyl)cyclo- butyl)amino)-2- hydroxypropoxy)phe- nyl)-N- methylmethanesulfon- amide | Calc'd 439.1 Found 439.1 | n/a | n/a |
| 66 | C | 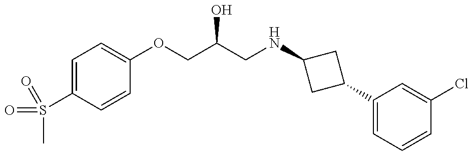 [SMILES: CS(=O)(=O)c1ccc(OC[C@@H](O)CN[C@H]2C[C@H](c3cccc(Cl)c3)C2)cc1] | (S)-1-((trans-3-(3- chlorophenyl)cyclo- butyl)amino)-3- (4- (methylsulfonyl)phe- noxy)propan-2-ol | Calc'd 410.1 Found 410.1 | n/a | n/a |
| 67 | D | 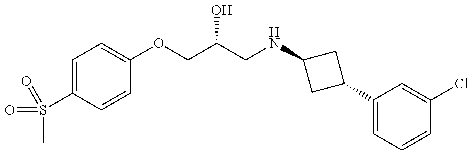 [SMILES: CS(=O)(=O)c1ccc(OC[C@H](O)CN[C@H]2C[C@H](c3cccc(Cl)c3)C2)cc1] | (R)-1-((trans-3-(3- chlorophenyl)cyclo- butyl)amino)-3- (4- (methylsulfonyl)phe- noxy)propan-2-ol | Calc'd 410.1 Found 410.1 | n/a | n/z |
| 68 | A | 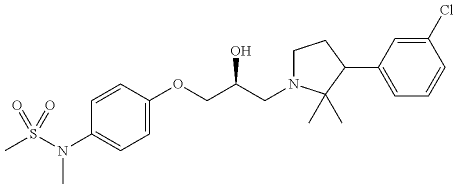 [SMILES: CN(c1ccc(OC[C@@H](O)CN2CCC(c3cccc(Cl)c3)C2(C)C)cc1)S(C)(=O)=O] | N-(4-((2S)-3-(3-(3- chlorophenyl)-2,2- dimethylpyrrolidin- 1-yl)-2- hydroxypropoxy)phe- nyl)-N- methylmethanesulfon- amide | Calc'd 467.2 Found 467.1 | n/a | n/a |
| 69 | A | 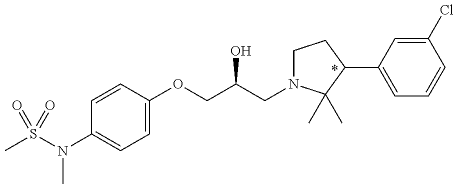 [SMILES: CN(c1ccc(OC[C@@H](O)CN2CCC(c3cccc(Cl)c3)C2(C)C)cc1)S(C)(=O)=O] | N-(4-((S)-3-((S) or (R)-3-(3- chlorophenyl)-2,2- dimethylpyrrolidin- 1-yl)-2- hydroxypropoxy)phe- nyl)-N- methylmethanesulfon- amide | Calc'd 467.2 Found 467.1 | Chiralcel OX- H | 1st |
| 70 | A | 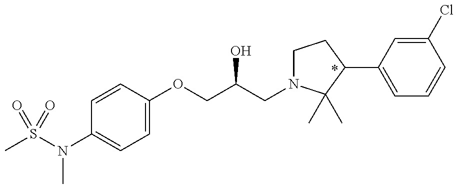 [SMILES: CN(c1ccc(OC[C@@H](O)CN2CCC(c3cccc(Cl)c3)C2(C)C)cc1)S(C)(=O)=O] | N-(4-((R)-(S)-3- ((R) or (S)-3-(3- chlorophenyl)-2,2- dimethylpyrrolidin- 1-yl)-2- hydroxypropoxy)phe- nyl)-N- methylmethanesulfon- amide | Calc'd 467.2 Found 467.1 | Chiralcel OX- H | 2nd |
| 71 | B | 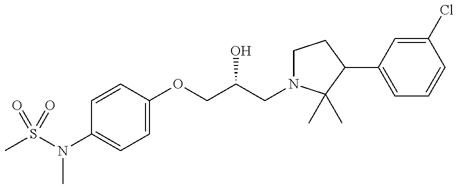 [SMILES: CN(c1ccc(OC[C@H](O)CN2CCC(c3cccc(Cl)c3)C2(C)C)cc1)S(C)(=O)=O] | N-(4-((2R)-3-(3- (3-chlorophenyl)- 2,2- dimethylpyrrolidin- 1-yl)-2- hydroxypropoxy)phe- nyl)-N- methylmethanesulfon- amide | Calc'd 467.2 Found 467.0 | n/a | n/a |
| 72 | B | 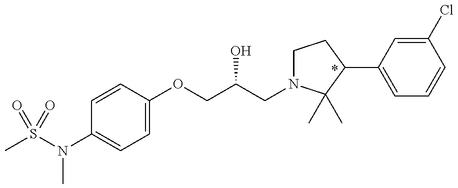 [SMILES: CN(c1ccc(OC[C@H](O)CN2CCC(c3cccc(Cl)c3)C2(C)C)cc1)S(C)(=O)=O] | N-(4-((R)-3-((S) or (R)-3-(3- chlorophenyl)-2,2- dimethylpyrrolidin- 1-yl)-2- hydroxypropoxy)phe- nyl)-N- methylmethanesulfon- amide | Calc'd 467.2 Found 467.1 | Chiralcel OX-H | 1st |
| 73 | B | 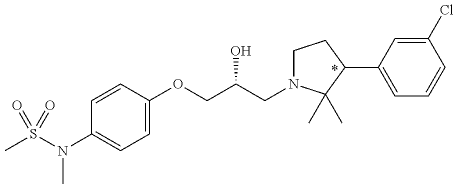 [SMILES: CN(c1ccc(OC[C@H](O)CN2CCC(c3cccc(Cl)c3)C2(C)C)cc1)S(C)(=O)=O] | N-(4-((R)-3-((R) or (S)-3-(3- chlorophenyl)-2,2- dimethylpyrrolidin- 1-yl)-2- hydroxypropoxy)phe- nyl)-N- methylmethanesulfon- amide | Calc'd 467.2 Found 467.1 | Chiralcel OX-H | 2nd |
| 74 | A | 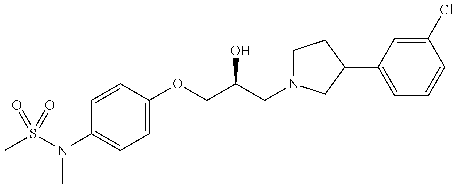 [SMILES: CN(c1ccc(OC[C@@H](O)CN2CCC(c3cccc(Cl)c3)C2)cc1)S(C)(=O)=O] | N-(4-((2S)-3-(3-(3- chlorophenyl)pyrro- lidin-1-yl)-2- hydroxypropoxy)phe- nyl)-N- methylmethanesulfon- amide | Calc'd 439.1 Found 439.1 | n/a | n/a |
| 75 | A | 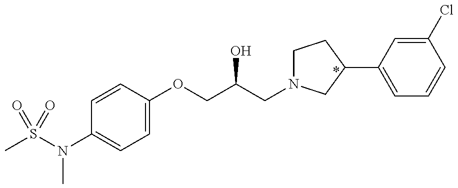 [SMILES: CN(c1ccc(OC[C@@H](O)CN2CCC(c3cccc(Cl)c3)C2)cc1)S(C)(=O)=O] | N-(4-((S)-3-((S) or (R)-3-(3- chlorophenyl)pyrro- lidin-1-yl)-2- hydroxypropoxy)phe- nyl)-N- methylmethanesulfon- amide | Calc'd 439.1 Found 439.2 | Chiralcel OD- H | 1st |
| 76 | A | 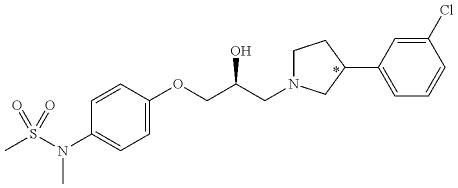 [SMILES: CN(c1ccc(OC[C@@H](O)CN2CCC(c3cccc(Cl)c3)C2)cc1)S(C)(=O)=O] | N-(4-((S)-3-((R) or (S)-3-(3- chlorophenyl)pyrro- lidin-1-yl)-2- hydroxypropoxy)phe- nyl)-N- methylmethanesulfon- amide | Calc'd 439.1 Found 439.1 | Chiralcel OD-H | 2nd |
| 77 | B | 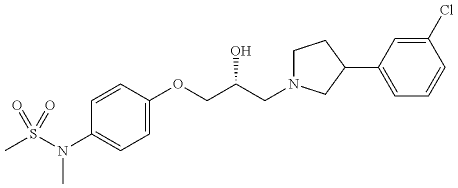 [SMILES: CN(c1ccc(OC[C@H](O)CN2CCC(c3cccc(Cl)c3)C2)cc1)S(C)(=O)=O] | N-(4-((2R)-3-(3- (3- chlorophenyl)pyrro- lidin-1-yl)-2- hydroxypropoxy)phe- nyl)-N- methylmethanesulfon- amide | Calc'd 439.1 Found 439.9 | n/a | n/a |
| 78 | B | 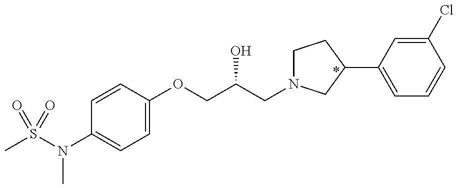 [SMILES: CN(c1ccc(OC[C@H](O)CN2CCC(c3cccc(Cl)c3)C2)cc1)S(C)(=O)=O] | N-(4-((R)-3-((S) or (R)-3-(3- chlorophenyl)pyrro- lidin-1-yl)-2- hydroxypropoxy)phe- nyl)-N- methylmethanesulfon- amide | Calc'd 439.1 Found 439.0 | Regis- pack | 1st |
| 79 | B | 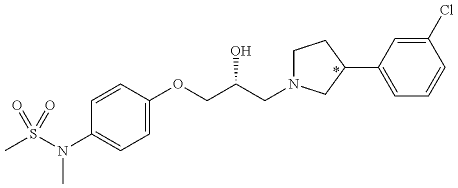 [SMILES: CN(c1ccc(OC[C@H](O)CN2CCC(c3cccc(Cl)c3)C2)cc1)S(C)(=O)=O] | N-(4-((R)-3-((R) or (S)-3-(3- chlorophenyl)pyrro- lidin-1-yl)-2- hydroxypropoxy)phe- nyl)-N- methylmethanesulfon- amide | Calc'd 439.1 Found 439.0 | Regis- pack | 2nd |
| 80 | A | 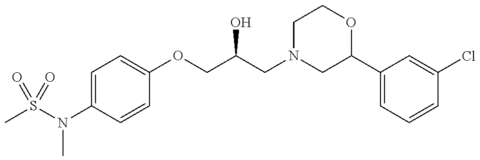 [SMILES: CN(c1ccc(OC[C@@H](O)CN2CCOC(c3cccc(Cl)c3)C2)cc1)S(C)(=O)=O] | N-(4-((2S)-3-(2-(3- chlorophenyl)morpho- lino)-2- hydroxypropoxy)phe- nyl)-N- methylmethanesulfon- amide | Calc'd 455.1 Found 455.1 | n/a | n/a |
| 81 | A | 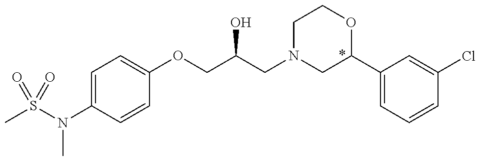 [SMILES: CN(c1ccc(OC[C@@H](O)CN2CCOC(c3cccc(Cl)c3)C2)cc1)S(C)(=O)=O] | N-(4-((S)-3-((S) or (R)-2-(3- chlorophenyl)morpho- lino)-2- hydroxypropoxy)phe- nyl)-N- methylmethanesulfon- amide | Calc'd 455.1 Found 454.9 | Chrome- ga Chiral CCA | 1st |
| 82 | A | 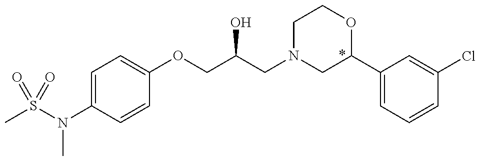 [SMILES: CN(c1ccc(OC[C@@H](O)CN2CCOC(c3cccc(Cl)c3)C2)cc1)S(C)(=O)=O] | N-(4-((S)-3-((R) or (S)-2-(3- chlorophenyl)morpho- lino)-2- hydroxypropoxy)phe- nyl)-N- methylmethanesulfon- amide | Calc'd 455.1 Found 454.9 | Chrome- ga Chiral CCA | 2nd |
| 83 | B | 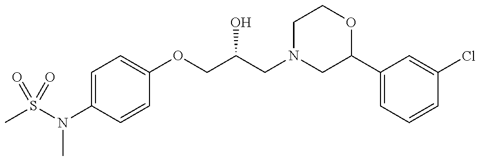 [SMILES: CN(c1ccc(OC[C@H](O)CN2CCOC(c3cccc(Cl)c3)C2)cc1)S(C)(=O)=O] | N-(4-((2R)-3-(2- (3- chlorophenyl)morpho- lino)-2- hydroxypropoxy)phe- nyl)-N- methylmethanesulfon- amide | Calc'd 455.1 Found 455.0 | n/a | n/a |
| 84 | B | 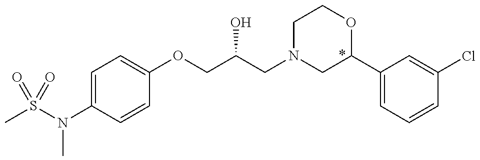 [SMILES: CN(c1ccc(OC[C@H](O)CN2CCOC(c3cccc(Cl)c3)C2)cc1)S(C)(=O)=O] | N-(4-((R)-3-((R) or (S)-2-(3- chlorophenyl)morpho- lino)-2- hydroxypropoxy)phe- nyl)-N- methylmethanesulfon- amide | Calc'd 455.1 Found 455.1 | Chrome- ga Chiral CCA | 1st |
| 85 | B | 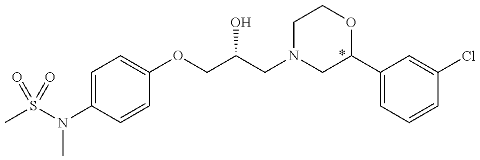 [SMILES: CN(c1ccc(OC[C@H](O)CN2CCOC(c3cccc(Cl)c3)C2)cc1)S(C)(=O)=O] | N-(4-((R)-3-((S) or (R)-2-(3- chlorophenyl)morpho- lino)-2- hydroxypropoxy)phe- nyl)-N- methylmethanesulfon- amide | Calc'd 455.1 Found 455.1 | Chrome ga Chiral CCA | 2nd |
| 86 | A | 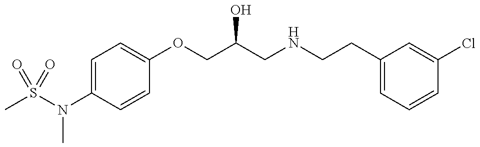 [SMILES: CN(c1ccc(OC[C@@H](O)CNCCc2cccc(Cl)c2)cc1)S(C)(=O)=O] | (S)-N-(4-(3-((3- chlorophenethyl)a- mino)-2- hydroxypropoxy)phe- nyl)-N- methylmethanesulfon- amide | Calc'd 413.1 Found 413.0 | n/a | n/a |
| 87 | B | 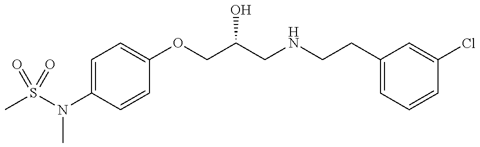 [SMILES: CN(c1ccc(OC[C@H](O)CNCCc2cccc(Cl)c2)cc1)S(C)(=O)=O] | (R)-N-(4-(3-((3- chlorophenethyl)a- mino)-2- hydroxypropoxy)phe- nyl)-N- methylmethanesulfon- amide | Calc'd 413.1 Found 413.0 | n/a | n/a |
| 88 | B | 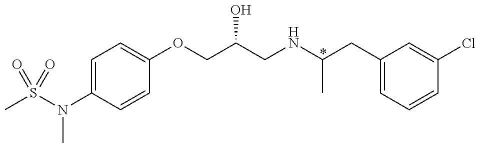 [SMILES: CC(Cc1cccc(Cl)c1)NC[C@@H](O)COc1ccc(N(C)S(C)(=O)=O)cc1] | N-(4-((R)-3-(((S) or (R)-1-(3- chlorophenyl)propan- 2-yl)amino)-2- hydroxypropoxy)phe- nyl)-N- methylmethanesulfon- amide | Calc'd 427.1 Found 427.1 | Chiralcel OX-H | 2nd |
| 89 | B | 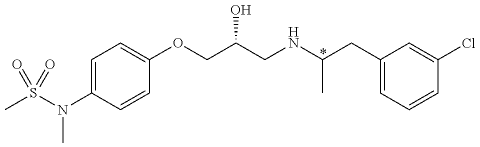 [SMILES: CC(Cc1cccc(Cl)c1)NC[C@@H](O)COc1ccc(N(C)S(C)(=O)=O)cc1] | N-(4-((R)-3-(((R) or (S)-1-(3- chlorophenyl)propan- 2-yl)amino)-2- hydroxypropoxy)phe- nyl)-N- methylmethanesulfon- amide | Calc'd 427.1 Found 427.1 | Chiralcel OX-H | 1st |
| 90 | A | 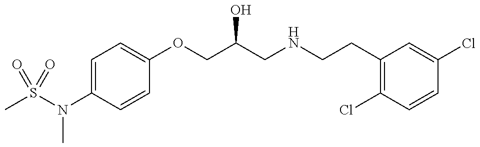 [SMILES: CN(c1ccc(OC[C@@H](O)CNCCc2cc(Cl)ccc2Cl)cc1)S(C)(=O)=O] | (S)-N-(4-(3-((2,5- dichlorophenethyl) amino)-2- hydroxypropoxy)phe- nyl)-N- methylmethanesulfon- amide | Calc'd 447.1 Found 447.0 | n/a | n/a |
| 91 | A | 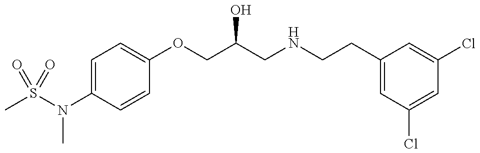 [SMILES: CN(c1ccc(OC[C@@H](O)CNCCc2cc(Cl)cc(Cl)c2)cc1)S(C)(=O)=O] | (S)-N-(4-(3-((3,5- dichlorophenethyl) amino)-2- hydroxypropoxy)phe- nyl)-N- methylmethanesulfon- amide | Calc'd 447.1 Found 447.0 | n/a | n/a |
| 92 | B | 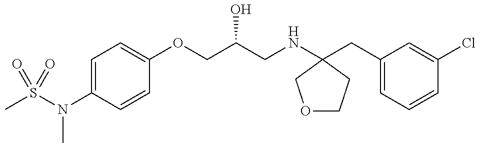 [SMILES: CN(c1ccc(OC[C@H](O)CNC2(Cc3cccc(Cl)c3)CCOC2)cc1)S(C)(=O)=O] | N-(4-((2R)-3-((3- (3- chlorobenzyl)tetra- hydrofuran-3- yl)amino)-2- hydroxypropoxy)phe- nyl)-N- methylmethanesulfon- amide | Calc'd 469.2 Found 469.1 | n/a | n/a |
| 93 | C | 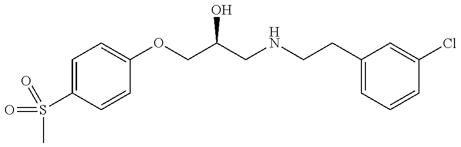 [SMILES: CS(=O)(=O)c1ccc(OC[C@@H](O)CNCCc2cccc(Cl)c2)cc1] | (S)-1-((3- chlorophenethyl)a- mino)-3-(4- (methylsulfonyl)phe- noxy)propan-2-ol | Calc'd 384.1 Found 384.1 | n/a | n/a |
| 94 | D | 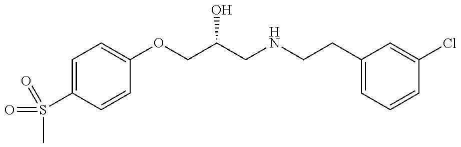 [SMILES: CS(=O)(=O)c1ccc(OC[C@H](O)CNCCc2cccc(Cl)c2)cc1] | (R)-1-((3- chlorophenethyl)a- mino)-3-(4- (methylsulfonyl)phe- noxy)propan-2-ol | Calc'd 384.1 Found 384.1 | n/a | n/a |
| 95 | A | 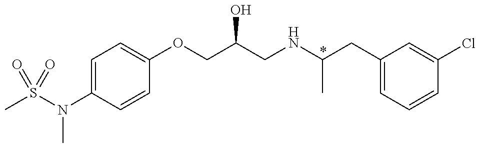 [SMILES: CC(Cc1cccc(Cl)c1)NC[C@H](O)COc1ccc(N(C)S(C)(=O)=O)cc1] | N-(4-((S)-3-(((S) or (R)-1-(3- chlorophenyl)propan- 2-yl)amino)-2- hydroxypropoxy)phe- nyl)-N- methylmethanesulfon- amide | Calc'd 427.1 Found 427.1 | Chrome- ga Chiral CCA | 2nd |
| 96 | A | 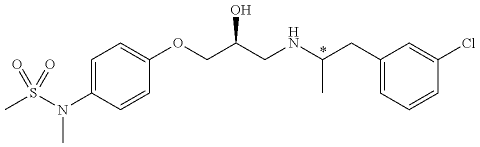 [SMILES: CC(Cc1cccc(Cl)c1)NC[C@H](O)COc1ccc(N(C)S(C)(=O)=O)cc1] | N-(4-((S)-3-(((R) or (S)-1-(3- chlorophenyl)propan- 2-yl)amino)-2- hydroxypropoxy)phe- nyl)-N- methylmethanesulfon- amide | Calc'd 427.1 Found 427.1 | Chrome- ga Chiral CCA | 1st |
| 97 | D | 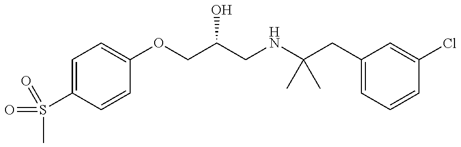 [SMILES: CC(C)(Cc1cccc(Cl)c1)NC[C@@H](O)COc1ccc(S(C)(=O)=O)cc1] | (R)-1-((1-(3- chlorophenyl)-2- methylpropan-2- yl)amino)-3-(4- (methylsulfonyl)phe- noxy)propan-2-ol | Calc'd 412.1 Found 412.0 | n/a | n/a |
| 98 | A | 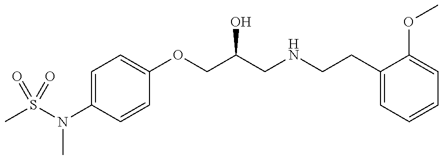 | (S)-N-(4-(2- hydroxy-3-((2- methoxyphenethyl) amino)propoxy)phe- nyl)-N- methylmethanesulfon- amide | Calc'd 409.2 Found 408.9 | n/a | n/a |
| 99 | A | 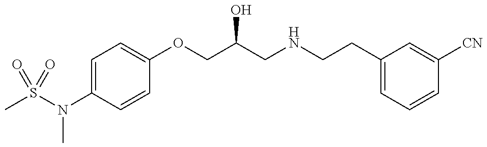 [SMILES: CN(c1ccc(OC[C@@H](O)CNCCc2cccc(C#N)c2)cc1)S(C)(=O)=O] | (S)-N-(4-(3-((3- cyanophenethyl)a- mino)-2- hydroxypropoxy)phe- nyl)-N- methylmethanesulfon- amide | Calc'd 404.2 Found 404.3 | n/a | n/a |
| 100 | A | 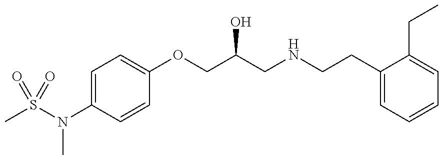 [SMILES: CCc1ccccc1CCNC[C@H](O)COc1ccc(N(C)S(C)(=O)=O)cc1] | (S)-N-(4-(3-((2- ethylphenethyl)ami- no)-2- hydroxypropoxy)phe- nyl)-N- methylmethanesulfon- amide | Calc'd 407.2 Found 407.2 | n/a | n/a |
| 101 | A | 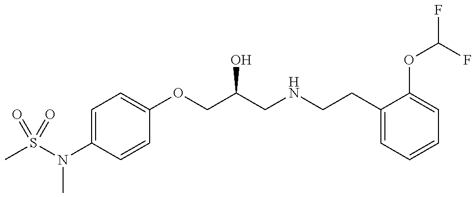 [SMILES: CN(c1ccc(OC[C@@H](O)CNCCc2ccccc2OC(F)F)cc1)S(C)(=O)=O] | (S)-N-(4-(3-((2- (difluoromethoxy) phenethyl)amino)- 2- hydroxypropoxy)phe- nyl)-N- methylmethanesulfon- amide | Calc'd 445.2 Found 445.1 | n/a | n/a |
| 102 | D | 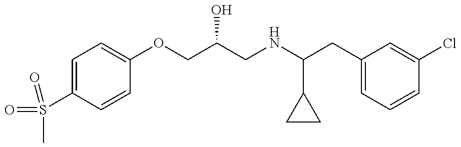 [SMILES: CS(=O)(=O)c1ccc(OC[C@H](O)CNC(Cc2cccc(Cl)c2)C2CC2)cc1] | (2R)-1-((2-(3- chlorophenyl)-1- cyclopropylethyl)a- mino)-3-(4- (methylsulfonyl)phe- noxy)propan-2-ol | Calc'd 424.1 Found 424.1 | n/a | n/a |
| 103 | E | 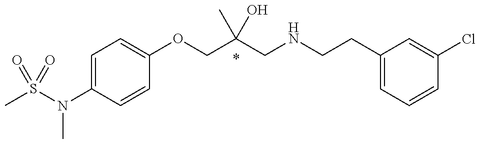 [SMILES: CN(c1ccc(OCC(C)(O)CNCCc2cccc(Cl)c2)cc1)S(C)(=O)=O] | (R) or (S)-N-(4-(3- ((3- chlorophenethyl)a- mino)-2-hydroxy- 2- methylpropxy)phe- nyl)-N- methylmethanesulfon- amide | Calc'd 427.1 Found 427.1 | Pheno- menex Cellulose- 2 | 1st |
| 104 | E | 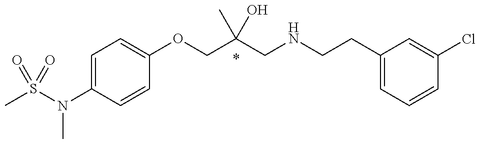 [SMILES: CN(c1ccc(OCC(C)(O)CNCCc2cccc(Cl)c2)cc1)S(C)(=O)=O] | (S) or (R)-N-(4-(3- ((3- chlorophenethyl)a- mino)-2-hydroxy- 2- methylpropoxy)phe- nyl)-N- methylmethanesulfon- amide | Calc'd 427.1 Found 427.1 | Pheno- menex Cellulose- 2 | 2nd |
| 105 | F | 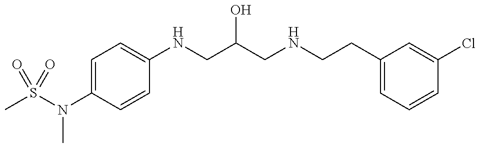 [SMILES: CN(c1ccc(NCC(O)CNCCc2cccc(Cl)c2)cc1)S(C)(=O)=O] | N-(4-((3-((3- chlorophenethyl)a- mino)-2- hydroxypropyl)ami- no)phenyl)-N- methylmethanesulfon- amide | Calc'd 412.1 Found 412.1 | n/a | n/a |
| 106 | F | 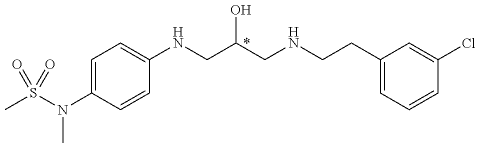 [SMILES: CN(c1ccc(NCC(O)CNCCc2cccc(Cl)c2)cc1)S(C)(=O)=O] | (R) or (S)-N-(4-((3- ((3- chlorophenethyl)a- mino)-2- hydroxypropyl)ami- no)phenyl)-N- methylmethanesulfon- amide | Calc'd 412.1 Found 412.1 | Chiralpak IG-3 | 2nd |

### Example 21

### 3-{2-[(2R,4S)-4-{[4-(azetidine-3-sulfonyl)phenoxy]methyl}-2-methylpyrrolidin-1-yl]ethyl}benzonitrile (Compound 156)

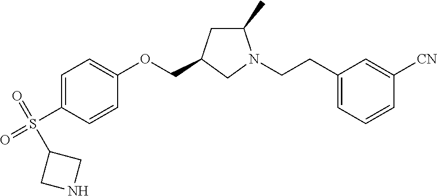 [SMILES: C[C@@H]1C[C@H](COc2ccc(S(=O)(=O)C3CNC3)cc2)CN1CCc1cccc(C#N)c1]

### Step 1: (2R,4S)-tert-butyl 4-((4-((1-((allyloxy)carbonyl)azetidin-3-yl)sulfonyl)phenoxy)methyl)-2-methylpyrrolidine-1-carboxylate

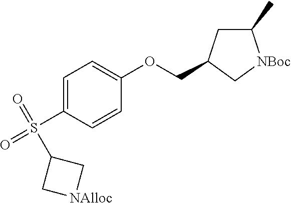

To a solution of allyl 3-((4-hydroxyphenyl)sulfonyl)azetidine-1-carboxylate (Intermediate AB, 400 mg, 1.35 mmol) in DMF (4 mL) were added K₂CO₃ (371.86 mg, 2.69 mmol) and tert-butyl (2R,4S)-2-methyl-4-(methylsulfonyloxymethyl)pyrrolidine-1-carboxylate (Intermediate AF, 434 mg, 1.48 mmol). The mixture was stirred at 80° C. for 16 h. The reaction mixture was partitioned between water (15 mL) and EtOAc (10 mL). The organic phase was separated, washed with brine (10 mL), dried over Na₂SO₄, filtered and concentrated under reduced pressure to give (2R,4S)-tert-butyl 4-((4-((1-((allyloxy)carbonyl)azetidin-3-yl)sulfonyl)phenoxy)methyl)-2-methylpyrrolidine-1-carboxylate. MS=395.1 [M-C₅H₉O₂+H]⁺

### Step 2: allyl 3-((4-(((3S,5R)-5-methylpyrrolidin-3-yl)methoxy)phenyl)sulfonyl)azetidine-1-carboxylate

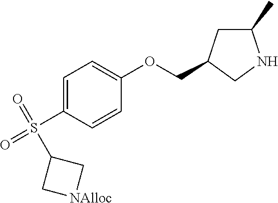

To a solution of (2R,4S)-tert-butyl 4-((4-((1-((allyloxy)carbonyl)azetidin-3-yl)sulfonyl)phenoxy)methyl)-2-methylpyrrolidine-1-carboxylate (730 mg, 1.48 mmol) in MeOH (2 mL) was added HCl/MeOH (4 M, 10 mL). The mixture was stirred at room temperature for 2 h. MeOH was removed under reduced pressure to give allyl 3-((4-(((3S,5R)-5-methylpyrrolidin-3-yl)methoxy)phenyl)sulfonyl)azetidine-1-carboxylate HCl salt. MS=395.2 [M+H]⁺.

### Step 3: allyl 3-[4-[[(3S,5R)-1-[2-(3-cyanophenyl)ethyl]-5-methyl-pyrrolidin-3-yl]methoxy]phenyl]sulfonylazetidine-1-carboxylate

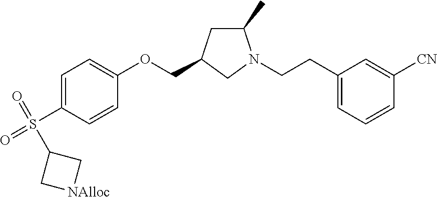

To a mixture of allyl 3-[4-[[(3S,5R)-5-methylpyrrolidin-3-yl]methoxy]phenyl]sulfonylazetidine-1-carboxylate (120 mg, 278 μmol, HCl salt), TEA (28.2 mg, 278 μmol) and 3-(2-oxoethyl)benzonitrile (52.6 mg, 362 μmol) in MeOH (5 mL) was added AcOH (16.7 mg, 278 μmol) followed by NaBH₃CN (35.0 mg, 557 μmol). The mixture was stirred at room temperature for 16 h. The reaction mixture was quenched by addition of water (5 mL) and adjusted to pH=7 with saturated aq NaHCO₃. The aqueous phase was extracted with EtOAc (10 mL×3). The combined organic layers were washed with brine (10 mL), dried over Na₂SO₄, filtered and concentrated under reduced pressure to give a residue. The residue was purified by flash silica gel chromatography (ISCO 4 g cartridge, 0-100% EtOAc:Hexane) to give allyl 3-[4-[[(3S,5R)-1-[2-(3-cyanophenyl)ethyl]-5-methyl-pyrrolidin-3-yl]methoxy]phenyl]sulfonylazetidine-1-carboxylate. MS=524.3 [M+H]⁺.

### Step 4: 3-{2-[(2R,4S)-4-{[4-(azetidine-3-sulfonyl)phenoxy]methyl}-2-methylpyrrolidin-1-yl]ethyl}benzonitrile (Compound 156)

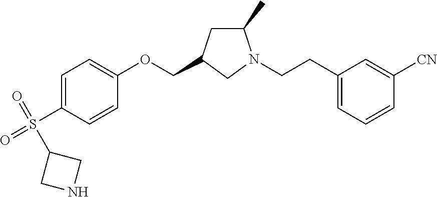

To a solution of allyl 3-[4-[[(3S,5R)-1-[2-(3-cyanophenyl)ethyl]-5-methyl-pyrrolidin-3-yl]methoxy]phenyl]sulfonylazetidine-1-carboxylate (60.0 mg, 115 μmol) in THF (5 mL) were added morpholine (39.9 mg, 458 μmol) and Pd(PPh₃)₄ (26.5 mg, 22.9 μmol). The mixture was then stirred at room temperature for 3 h. The reaction mixture was filtered and concentrated under reduced pressure to give a residue. The residue was purified by flash silica gel chromatography (ISCO 4 g cartridge, 0-100% EtOAc:Hexane, then 0-20% MeOH:EtOAc) to give a crude product. The product was further purified by preparative reverse phase HPLC (Waters Xbridge BEH C18 column, 25-55% MeCN:10 mM NH₄HCO₃ in H₂O) to give 3-{2-[(2R,4S)-4-{[4-(azetidine-3-sulfonyl)phenoxy]methyl}-2-methylpyrrolidin-1-yl]ethyl}benzonitrile (Compound 156). ¹H NMR (400 MHz, DMSO-d₆): δ 7.78 (d, J=8.8 Hz, 2H), 7.72 (s, 1H), 7.64-7.57 (m, 2H), 7.42 (t, J=7.6 Hz, 1H), 7.16 (d, J=8.8 Hz, 2H), 4.47-7.39 (m, 1H), 3.90 (d, J=7.2 Hz, 2H), 3.72 (t, J=8.0 Hz, 2H), 3.49 (t, J=8.4 Hz, 2H), 3.07 (d, J=8.0 Hz, 1H), 2.99-2.96 (m, 1H), 2.81-2.75 (m, 2H), 2.35-2.30 (m, 5H), 2.28-2.12 (m, 1H), 1.07-1.02 (m, 1H), 0.98 (d, J=6.0 Hz, 1H). MS=440.1 [M+H]⁺.

The following compounds in Table 22 were prepared according to procedures similar to those described for Compound 156 using the appropriate starting materials.

TABLE 22

| | | | Exact | | Chiral |
|---|---|---|---|---|---|
| | | | Mass | Chiral | Elution |
| No. | Structure | IUPAC Name | [M + H]⁺ | column | order |
| 157 | 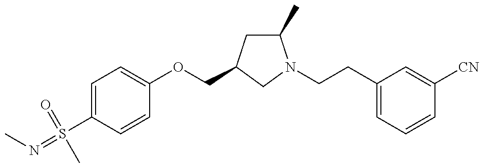 [SMILES: CN=S(C)(=O)c1ccc(OC[C@H]2C[C@@H](C)N(CCc3cccc(C#N)c3)C2)cc1] | 3-[2-[(2R,4S)-2- methyl-4-({4- [methyl(methyli- mino)oxo-λ⁶- sulfanyl]phenoxy} methyl)pyrrolidin- 1- yl]ethyl}benzonitrile | Calc'd 412.2 Found 412.3 | n/a | n/a |
| 158 | 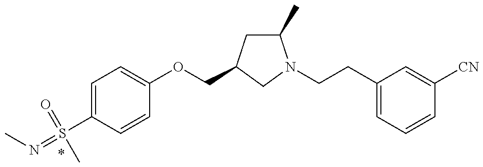 [SMILES: CN=S(C)(=O)c1ccc(OC[C@H]2C[C@@H](C)N(CCc3cccc(C#N)c3)C2)cc1] | 3-{2-[(2R,4S)-2- methyl-4-({4-[(R or S)methyl(methyli- mino)oxo-λ⁶- sulfanyl]phenoxy} methyl)pyrrolidin- 1- yl]ethyl}benzonitrile | Calc'd 412.2 Found 412.3 | Chiralpak AD | 1st |
| 159 | 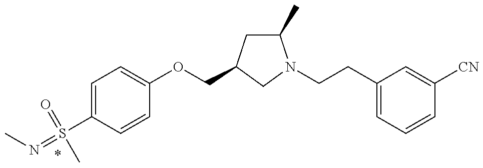 [SMILES: CN=S(C)(=O)c1ccc(OC[C@H]2C[C@@H](C)N(CCc3cccc(C#N)c3)C2)cc1] | 3-{2-[(2R,4S)-2- methyl-4-({4-[(S or R)methyl(methyli- mino)oxo-λ⁶- sulfanyl]phenoxy} methyl)pyrrolidin- 1- yl]ethyl}benzonitrile | Calc'd 412.2 Found 412.3 | Chiralpak AD | 2nd |
| 161 | 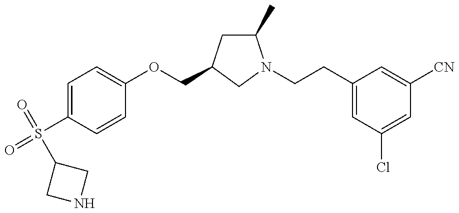 [SMILES: C[C@@H]1C[C@H](COc2ccc(S(=O)(=O)C3CNC3)cc2)CN1CCc1cc(Cl)cc(C#N)c1] | 3-{2-[(2R,4S)-4- {[4-(azetidine-3- sulfonyl)phenoxy] methyl}-2- methylpyrrolidin- 1-yl]ethyl}-5- chlorobenzonitrile | Calc'd 474.2 Found 474.2 | n/a | n/a |
| 162 | 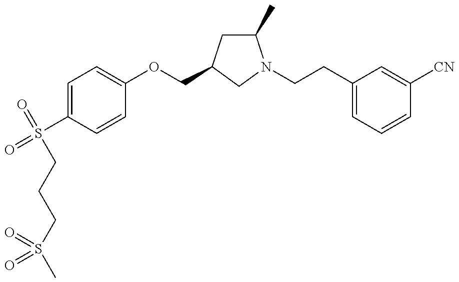 [SMILES: C[C@@H]1C[C@H](COc2ccc(S(=O)(=O)CCCS(C)(=O)=O)cc2)CN1CCc1cccc(C#N)c1] | 3-{2-[(2R,4S)-4- {[4-(3- methanesulfonylpro- panesulfonyl)phe- noxy]methyl}- 2- methylpyrrolidin- 1- yl]ethyl}benzonitrile | Calc'd 505.2 Found 505.3 | n/a | n/a |
| 164 | 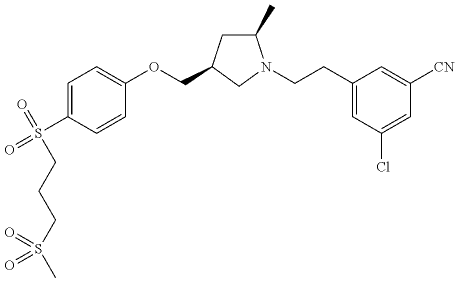 [SMILES: C[C@@H]1C[C@H](COc2ccc(S(=O)(=O)CCCS(C)(=O)=O)cc2)CN1CCc1cc(Cl)cc(C#N)c1] | 3-chloro-5-{2- [(2R,4S)-4-{[4- (3- methanesulfonylpro- panesulfonyl)phe- noxy]methyl}- 2- methylpyrrolidin- 1- yl]ethyl}benzonitrile | Calc'd 539.1 Found 539.2 | n/a | n/a |
| 165 | 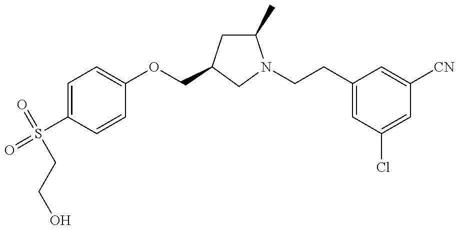 [SMILES: C[C@@H]1C[C@H](COc2ccc(S(=O)(=O)CCO)cc2)CN1CCc1cc(Cl)cc(C#N)c1] | 3-chloro-5-{2- [(2R,4S)-4-{[4- (2- hydroxyethanesul- fonyl)phenoxy]meth- yl}-2- methylpyrrolidin- 1- yl]ethyl}benzonitrile | Calc'd 463.1 Found 463.2 | n/a | n/a |
| 166 | 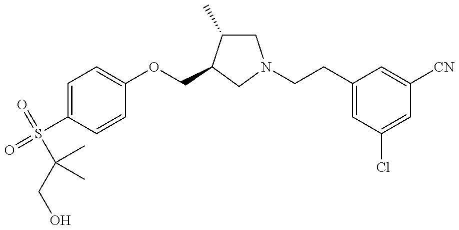 [SMILES: C[C@@H]1CN(CCc2cc(Cl)cc(C#N)c2)C[C@H]1COc1ccc(S(=O)(=O)C(C)(C)CO)cc1] | 3-chloro-5-{2- [(3S,4S)-3-{[4- (1-hydroxy-2- methylpropane-2- sulfonyl)phenoxy] methyl}-4- methylpyrrolidin- 1- yl]ethyl}benzonitrile | Calc'd 491.2 Found 491.1 | n/a | n/a |
| 167 | 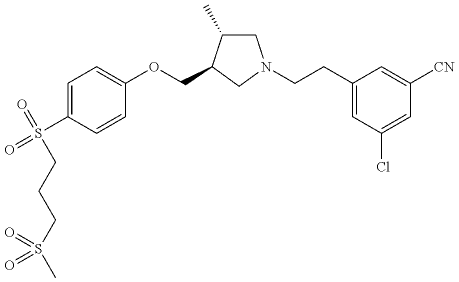 [SMILES: C[C@@H]1CN(CCc2cc(Cl)cc(C#N)c2)C[C@H]1COc1ccc(S(=O)(=O)CCCS(C)(=O)=O)cc1] | 3-chloro-5-{2- [(3S,4S)-3-{[4- (3- methanesulfonylpro- panesulfonyl)phe- noxy]methyl}- 4- methylpyrrolidin- 1- yl]ethyl}benzonitrile | Calc'd 539.1 Found 539.2 | n/a | n/a |
| 168 | 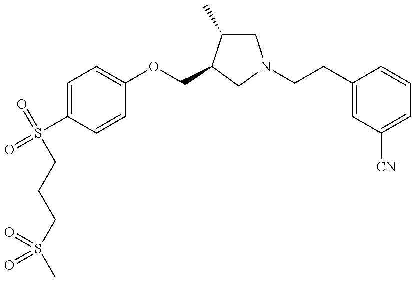 [SMILES: C[C@@H]1CN(CCc2cccc(C#N)c2)C[C@H]1COc1ccc(S(=O)(=O)CCCS(C)(=O)=O)cc1] | 3-{2-[(3S,4S)-3- {[4-(3- methanesulfonylpro- panesulfonyl)phe- noxy]methyl}- 4- methylpyrrolidin- 1- yl]ethyl}benzonitrile | Calc'd 505.2 Found 505.5 | n/a | n/a |
| 169 | 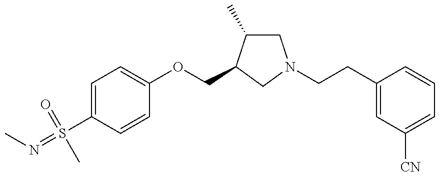 [SMILES: CN=S(C)(=O)c1ccc(OC[C@@H]2CN(CCc3cccc(C#N)c3)C[C@H]2C)cc1] | 3-{2-[(3S,4S)-3- methyl-4-({4- [methyl(methyli- mino)oxo-λ⁶- sulfanyl]phenoxy} methyl)pyrrolidin- 1- yl]ethyl}benzonitrile | Calc'd 412.2 Found 412.3 | n/a | n/a |
| 170 | 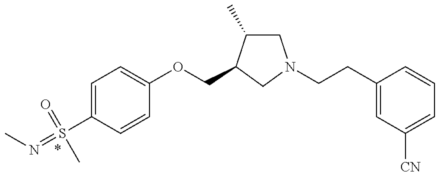 [SMILES: CN=S(C)(=O)c1ccc(OC[C@@H]2CN(CCc3cccc(C#N)c3)C[C@H]2C)cc1] | 3-{2-[(3S,4S)-3- methyl-4-({4-[(R or S)- methyl(methylimi- no)oxo-λ⁶- sulfanyl]phenoxy} methyl)pyrrolidin- 1- yl]ethyl}benzonitrile | Calc'd 412.2 Found 412.3 | Chiralpak IC | 1st |
| 171 | 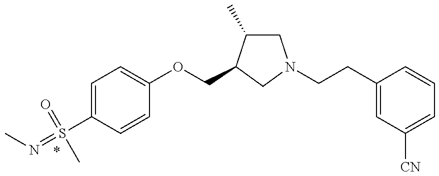 [SMILES: CN=S(C)(=O)c1ccc(OC[C@@H]2CN(CCc3cccc(C#N)c3)C[C@H]2C)cc1] | 3-{2-[(3S,4S)-3- methyl-4-({4-[(S or R)- methyl(methylimi- no)oxo-λ⁶- sulfanyl]phenoxy} methyl)pyrrolidin- 1- yl]ethyl}benzonitrile | Calc'd 412.2 Found 412.2 | Chiralpak IC | 2nd |
| 172 | 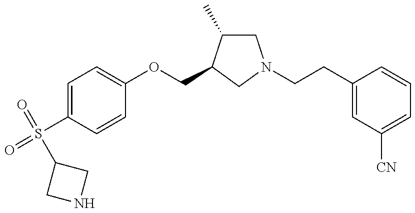 [SMILES: C[C@@H]1CN(CCc2cccc(C#N)c2)C[C@H]1COc1ccc(S(=O)(=O)C2CNC2)cc1] | 3-{2-[(3S,4S)-3- {[4-(azetidine-3- sulfonyl)phenoxy] methyl}-4- methylpyrrolidin- 1- yl]ethyl}benzonitrile | Calc'd 440.2 Found 440.1 | n/a | n/a |
| 173 | 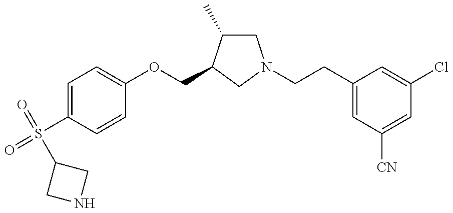 [SMILES: C[C@@H]1CN(CCc2cc(Cl)cc(C#N)c2)C[C@H]1COc1ccc(S(=O)(=O)C2CNC2)cc1] | 3-{2-[(3S,4S)-3- {[4-(azetidine-3- sulfonyl)phenoxy] methyl}-4- methylpyrrolidin- 1-yl]ethyl}-5- chlorobenzonitrile | Calc'd 474.2 Found 474.2 | n/a | n/a |
| 174 | 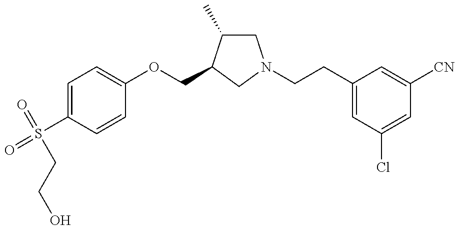 [SMILES: C[C@@H]1CN(CCc2cc(Cl)cc(C#N)c2)C[C@H]1COc1ccc(S(=O)(=O)CCO)cc1] | 3-chloro-5-{2- [(3S,4S)-3-{[4- (2- hydroxyethanesul- fonyl)phenoxy]meth- yl}-4- methylpyrrolidin- 1- yl]ethyl}benzonitrile | Calc'd 463.1 Found 463.1 | n/a | n/a |
| 175 | 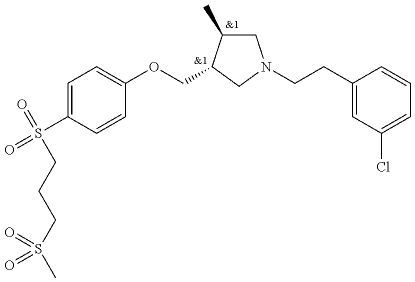 [SMILES: C[C@H]1CN(CCc2cccc(Cl)c2)C[C@@H]1COc1ccc(S(=O)(=O)CCCS(C)(=O)=O)cc1] | rac-trans-1-[2-(3- chlorophenyl)eth- yl]-3-{[4-(3- methanesulfonylpro- panesulfonyl)phe- noxy]methyl}- 4- methylpyrrolidine | Calc'd 514.1 Found 514.1 | n/a | n/a |
| 176 | 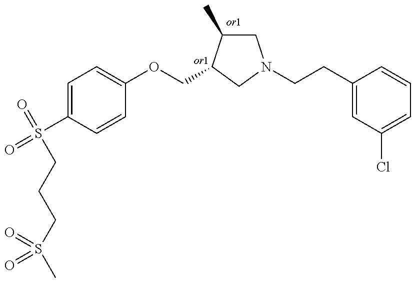 [SMILES: C[C@H]1CN(CCc2cccc(Cl)c2)C[C@@H]1COc1ccc(S(=O)(=O)CCCS(C)(=O)=O)cc1] | (3R,4R or 3S,4S)- 1-[2-(3- chlorophenyl)eth- yl]-3-{[4-(3- methanesulfonylpro- panesulfonyl)phe- noxy]methyl}- 4- methylpyrrolidine | Calc'd 514.1 Found 514.2 | Chiralpak AD | 1st |
| 177 | 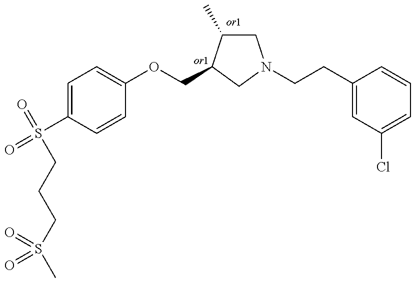 [SMILES: C[C@@H]1CN(CCc2cccc(Cl)c2)C[C@H]1COc1ccc(S(=O)(=O)CCCS(C)(=O)=O)cc1] | (3S,4S or 3R,4R)- 1-[2-(3- chlorophenyl)eth- yl]-3-{[4-(3- methanesulfonylpro- panesulfonyl)phe- noxy]methyl}- 4- methylpyrrolidine | Calc'd 514.1 Found 514.2 | Chiralpak AD | 2nd |

### Example 22

### (3S)-1-[2-(3-chlorophenyl)ethyl]-3-{[4-(3-methanesulfonylpropanesulfonyl)phenoxy]methyl}piperazine (Compound 184)

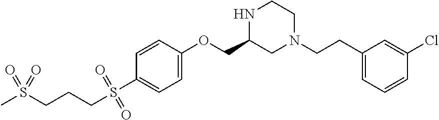 [SMILES: CS(=O)(=O)CCCS(=O)(=O)c1ccc(OC[C@@H]2CN(CCc3cccc(Cl)c3)CCN2)cc1]

### Step 1: (S)-tert-butyl 4-(3-chlorophenethyl)-2-(hydroxymethyl)piperazine-1-carboxylate

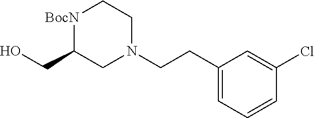 [SMILES: CC(C)(C)OC(=O)N1CCN(CCc2cccc(Cl)c2)C[C@H]1CO]

To a mixture of tert-butyl (2S)-2-(hydroxymethyl)piperazine-1-carboxylate (3.0 g, 13.9 mmol) and 2-(3-chlorophenyl)acetaldehyde (2.79 g, 18.0 mmol) in MeOH (30 mL) and AcOH (1.5 mL) was added 2-methylpyridine borane complex (1.78 g, 16.6 mmol). The mixture was stirred at 40° C. for 2 h. The reaction mixture was concentrated under reduced pressure. The residue was diluted with water (50 mL), adjusted to pH=7 with 2 M aqueous NaHCO₃ and extracted with EtOAc (50 mL×3). The combined organic layers were washed with brine (50 mL), dried over Na₂SO₄, filtered and concentrated under reduced pressure to give a residue. The residue was purified by flash silica gel chromatography (Biotage 40 g cartridge, 0-100% EtOAc:Hexane) to give (S)-tert-butyl 4-(3-chlorophenethyl)-2-(hydroxymethyl)piperazine-1-carboxylate. MS=355.3 [M+H]⁺.

### Step 2: (S)-(4-(3-chlorophenethyl)piperazin-2-yl)methanol

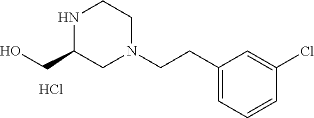 [SMILES: OC[C@@H]1CN(CCc2cccc(Cl)c2)CCN1 ; Cl]

A solution of (S)-tert-butyl 4-(3-chlorophenethyl)-2-(hydroxymethyl)piperazine-1-carboxylate (3.50 g, 9.86 mmol) in HCl/MeOH (4 M, 20 mL) was stirred at room temperature for 2 h. The reaction mixture was concentrated under reduced pressure to give (S)-(4-(3-chlorophenethyl)piperazin-2-yl)methanol HCl salt, which was used without further purification. MS=255.1 [M+H]⁺.

### Step 3: (3aS)-5-(3-chlorophenethyl)hexahydro-[1,2,3]oxathiazolo[3,4-a]pyrazine 1-oxide

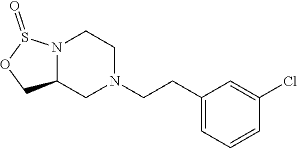

To a solution of (S)-(4-(3-chlorophenethyl)piperazin-2-yl)methanol (2.50 g, 8.58 mmol, HCl salt) in DCM (30 mL) were added imidazole (1.75 g, 25.7 mmol) and TEA (2.17 g, 21.5 mmol). The mixture was cooled to 0° C., and SOCl₂ (1.53 g, 12.9 mmol) was added. The mixture was stirred at room temperature for 16 h. The reaction mixture was quenched by addition of water (30 mL) and extracted with DCM (30 mL×3). The combined organic layers were washed with brine (30 mL), dried over Na₂SO₄, filtered and concentrated under reduced pressure to give a residue. The residue was purified by flash silica gel chromatography (Biotage 40 g cartridge, 0-100% EtOAc:Hexane) to give (3aS)-5-(3-chlorophenethyl)hexahydro-[1,2,3]oxathiazolo[3,4-a]pyrazine 1-oxide. MS=301.0 [M+H]⁺.

### Step 4: (S)-5-(3-chlorophenethyl)hexahydro-[1,2,3]oxathiazolo[3,4-a]pyrazine 1,1-dioxide

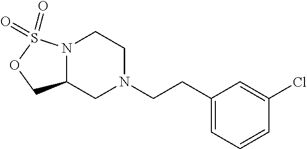 [SMILES: O=S1(=O)OC[C@@H]2CN(CCc3cccc(Cl)c3)CCN21]

To a solution of (3aS)-5-(3-chlorophenethyl)hexahydro-[1,2,3]oxathiazolo[3,4-a]pyrazine 1-oxide (1.80 g, 5.98 mmol) in MeCN (40 mL) were added RuCl₃ (13 mg, 59.8 μmol) and a solution of NaIO₄ (1.92 g, 8.98 mmol) in water (10 mL) under N₂. The mixture was stirred at room temperature for 2 h. The reaction mixture was quenched by saturated aqueous Na₂SO₃ (100 mL), then was diluted with water (40 mL) and extracted with EtOAc (30 mL×3). The combined organic layers were washed with brine (50 mL), dried over Na₂SO₄, filtered and concentrated under reduced pressure to give a residue. The residue was purified by flash silica gel chromatography (Biotage 20 g cartridge, 0-100% EtOAc:Hexane) to give (S)-5-(3-chlorophenethyl)hexahydro-[1,2,3]oxathiazolo[3,4-a]pyrazine 1,1-dioxide. MS=317.0 [M+H]⁺.

### Step 5: (3S)-1-[2-(3-chlorophenyl)ethyl]-3-{[4-(3-methanesulfonylpropanesulfonyl)phenoxy]methyl}piperazine (Compound 184)

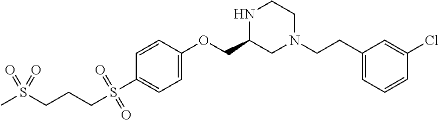 [SMILES: CS(=O)(=O)CCCS(=O)(=O)c1ccc(OC[C@@H]2CN(CCc3cccc(Cl)c3)CCN2)cc1]

To a mixture of 4-(3-methanesulfonylpropanesulfonyl)phenol (Intermediate AD, 48 mg, 174 μmol) and (S)-5-(3-chlorophenethyl)hexahydro-[1,2,3]oxathiazolo[3,4-a]pyrazine 1,1-dioxide (50 mg, 158 μmol) in DMF (3 mL) was added K₂CO₃ (33 mg, 237 μmol). The reaction was stirred at 60° C. for 16 h. The reaction mixture was filtered and the filtrate was quenched with 3.0 M aqueous HCl (106 μL). The mixture was stirred for 2 h, solids were removed by filtration, and the filtrate was concentrated in vacuo. The residue was purified by preparative reverse phase HPLC (Phenomenex Gemini-NX, 18-48% MeCN:10 mM NH₄HCO₃ in H₂O) to give (3S)-1-[2-(3-chlorophenyl)ethyl]-3-{[4-(3-methanesulfonylpropanesulfonyl)phenoxy]methyl}piperazine (Compound 184). ¹H NMR (400 MHz, DMSO-d₆): δ=7.81 (d, J=8.8 Hz, 2H), 7.32-7.31 (m, 1H), 7.29-7.27 (m, 1H), 7.24-7.18 (m, 4H), 4.02-3.97 (m, 2H), 3.42-3.38 (m, 3H), 3.20 (t, J=8.0 Hz, 2H), 3.02-3.00 (m, 1H), 2.96 (s, 3H), 2.87 (t, J=9.2 Hz, 2H), 2.76-2.66 (m, 5H), 2.06-1.87 (m, 5H). MS=515.1 [M+H]⁺.

The following compounds in Table 23 were prepared according to procedures similar to those described for Compound 184 using the appropriate starting materials.

TABLE 23

| | | | Exact | | Chiral |
|---|---|---|---|---|---|
| | | | Mass | Chiral | eluting |
| No. | Structure | IUPAC Name | [M + H]⁺ | column | order |
| 182 | 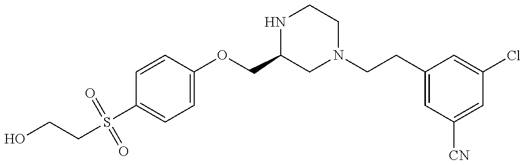 [SMILES: N#Cc1cc(Cl)cc(CCN2CCN[C@H](COc3ccc(S(=O)(=O)CCO)cc3)C2)c1] | 3-chloro-5-{2- [(3S)-3-{[4-(2- hydroxyethane- sulfonyl)phenoxy] methyl}pi- perazin-1- yl]ethyl}benzo- nitrile | Calc'd 464.1 Found 464.1 | n/a | n/a |
| 183 | 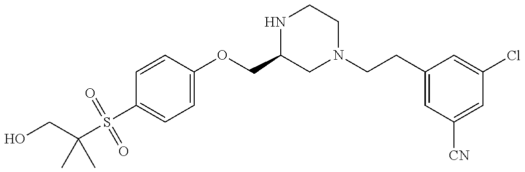 [SMILES: CC(C)(CO)S(=O)(=O)c1ccc(OC[C@@H]2CN(CCc3cc(Cl)cc(C#N)c3)CCN2)cc1] | 3-chloro-5-{2- [(3S)-3-{[4-(1- hydroxy-2- methylpropane- 2- sulfonyl)phenoxy] methyl}pi- perazin-1- yl]ethyl}benzo- nitrile | Calc'd 492.2 Found 492.1 | n/a | n/a |
| 185 | 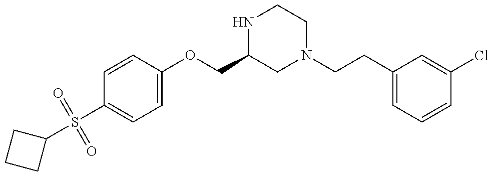 [SMILES: O=S(=O)(c1ccc(OC[C@@H]2CN(CCc3cccc(Cl)c3)CCN2)cc1)C1CCC1] | (3S)-3-{[4- (azetidine-3- sulfonyl)phenoxy] methyl}- 1-[2-(3- chlorophenyl) ethyl]piperazine | Calc'd 450.2 Found 450.1 | n/a | n/a |
| 186 | 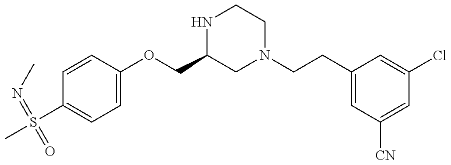 [SMILES: CN=S(C)(=O)c1ccc(OC[C@@H]2CN(CCc3cc(Cl)cc(C#N)c3)CCN2)cc1] | 3-chloro-5-{2- [(3S)-3-({4- [methyl(methyl- imino)oxo-λ⁶- sulfanyl]pheno- xy}methyl)pipera- zin-1- yl]ethyl}benzo- nitrile | Calc'd 447.2 Found 447.2 | n/a | n/a |
| 187 | 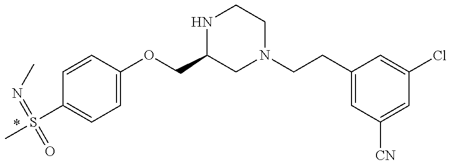 [SMILES: CN=S(C)(=O)c1ccc(OC[C@@H]2CN(CCc3cc(Cl)cc(C#N)c3)CCN2)cc1] | 3-chloro-5-{2- [(3S)-3-({4-[(S or R)- methyl(methyl- imino)oxo-λ⁶- sulfanyl]pheno- xy}methyl)pipera- zin-1- yl]ethyl}benzo- nitrile | Calc'd 447.2 Found 447.2 | Chiralpak IC | 1st |
| 188 | 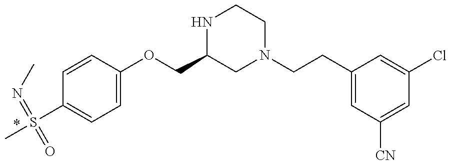 [SMILES: CN=S(C)(=O)c1ccc(OC[C@@H]2CN(CCc3cc(Cl)cc(C#N)c3)CCN2)cc1] | 3-chloro-5-{2- [(3S)-3-({4- [(R or S)- methyl(methyl- imino)oxo-λ⁶- sulfanyl]pheno- xy}methyl)piper- azin-1- yl]ethyl}benzo- nitrile | Calc'd 447.2 Found 447.1 | Chiralpak IC | 2nd |

### Example 23

### 3-chloro-5-{2-[(3S)-3-[(4-methanesulfonylphenoxy)methyl]piperazin-1-yl]ethyl}benzonitrile (Compound 189)

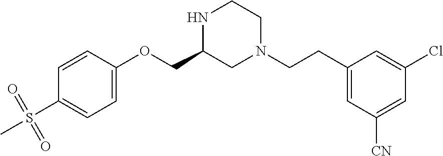 [SMILES: CS(=O)(=O)c1ccc(OC[C@@H]2CN(CCc3cc(Cl)cc(C#N)c3)CCN2)cc1]

### Step 1: (S)-4-benzyl 1-tert-butyl 2-(hydroxymethyl)piperazine-1,4-dicarboxylate

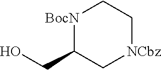 [SMILES: CC(C)(C)OC(=O)N1CCN(C(=O)OCc2ccccc2)C[C@H]1CO]

To a mixture of (S)-tert-butyl 2-(hydroxymethyl)piperazine-1-carboxylate (4.00 g, 18.5 mmol) and NaHCO₃ (4.66 g, 55.5 mmol) in THF (25 mL) and H₂O (25 mL) was added CbzCl (4.73 g, 27.7 mmol) at 0° C. Then the mixture was stirred at room temperature for 3 h. The mixture was extracted with EtOAc (30 mL×3). The combined organic layers were washed with brine (50 mL), dried over Na₂SO₄, filtered and concentrated under reduced pressure. The residue was purified by flash silica gel chromatography (Biotage 40 g cartridge, 0-40% EtOAc:Hexane) to give (S)-4-benzyl 1-tert-butyl 2-(hydroxymethyl)piperazine-1,4-dicarboxylate. MS=251.1 [M-C₅H₉O₂+H]⁺

### Step 2: (S)-4-benzyl 1-tert-butyl 2-((4-(methylsulfonyl)phenoxy)methyl)piperazine-1,4-dicarboxylate

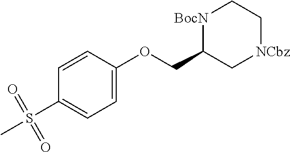 [SMILES: CC(C)(C)OC(=O)N1CCN(C(=O)OCc2ccccc2)C[C@H]1COc1ccc(S(C)(=O)=O)cc1]

To a solution of (S)-4-benzyl 1-tert-butyl 2-(hydroxymethyl)piperazine-1,4-dicarboxylate (450 mg, 1.28 mmol) and 4-(methylsulfonyl)phenol (G5, 221 mg, 1.28 mmol) in THF (8 mL) at room temperature were added tributylphosphine (520 mg, 2.57 mmol) and TMAD (442 mg, 2.57 mmol). The mixture was then stirred at 40° C. for 16 h under N₂. Brine was then added (10 mL) and the mixture was extracted with EtOAc (8 mL×3). The combined organic layers were dried over Na₂SO₄, filtered and concentrated under reduced pressure. The residue was purified by flash silica gel chromatography (Biotage 12 g cartridge, 0-100% EtOAc:Hexane) to give (S)-4-benzyl 1-tert-butyl 2-((4-(methylsulfonyl)phenoxy)methyl)piperazine-1,4-dicarboxylate which was taken to the next step without further purification. MS=449.1 [M-C₄H₈+H]⁺.

### Step 3: (S)-tert-butyl 2-((4-(methylsulfonyl)phenoxy)methyl)piperazine-1-carboxylate

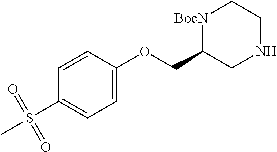 [SMILES: CC(C)(C)OC(=O)N1CCNC[C@H]1COc1ccc(S(C)(=O)=O)cc1]

To a suspension of Pd/C (100 mg, 10% by weight in mineral oil) in MeOH (15 mL) was added (S)-4-benzyl 1-tert-butyl 2-((4-(methylsulfonyl)phenoxy)methyl)piperazine-1,4-dicarboxylate (500 mg, 991 μmol). The suspension was degassed under vacuum and purged with H₂ three times. The mixture was then stirred under H₂ (15 psi) at room temperature for 1 h. The mixture was filtered through a pad of Celite, and the filter cake was rinsed with MeOH (8 mL×3). The combined filtrates were concentrated under reduced pressure. The residue was dissolved in saturated citric acid solution (30 mL) and extracted with EtOAc (10 mL×3). The aqueous layer was adjusted to pH=8 to 9 with saturated aqueous NaHCO₃, and then extracted with EtOAc (20 mL×2). The combined organic layers were dried over Na₂SO₄, filtered and concentrated under reduced pressure to give (S)-tert-butyl 2-((4-(methylsulfonyl)phenoxy)methyl)piperazine-1-carboxylate, which was taken to the next step without further purification. MS=271.1 [M-C₅H₉O₂+H]⁺.

### Step 4: (S)-tert-butyl 4-(3-chloro-5-cyanophenethyl)-2-((4-(methylsulfonyl)phenoxy) methyl)piperazine-1-carboxylate

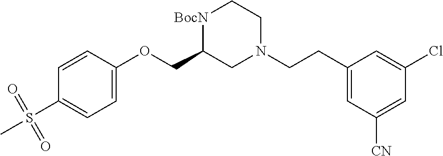 [SMILES: CC(C)(C)OC(=O)N1CCN(CCc2cc(Cl)cc(C#N)c2)C[C@H]1COc1ccc(S(C)(=O)=O)cc1]

To a mixture of (S)-tert-butyl 2-((4-(methylsulfonyl)phenoxy)methyl)piperazine-1-carboxylate (500 mg, 1.35 mmol) and 3-chloro-5-(2-oxoethyl)benzonitrile (606 mg, 3.37 mmol) in MeOH (10 mL) and HOAc (0.5 mL) was added NaBH₃CN (170 mg, 2.70 mmol) at 0° C. The mixture was then stirred at room temperature for 2 h under N₂. The reaction was quenched by addition of water (10 mL) at 0° C., and then adjusted to pH=7 with saturated aqueous NaHCO₃ and extracted with EtOAc (15 mL×3). The combined organic layers were washed with brine (30 mL×2), dried over Na₂SO₄, filtered and concentrated under reduced pressure to give a residue. The residue was purified by flash silica gel chromatography (Biotage 12 g cartridge, 0-40% EtOAc:Hexane) to give (S)-tert-butyl 4-(3-chloro-5-cyanophenethyl)-2-((4-(methylsulfonyl)phenoxy) methyl)piperazine-1-carboxylate. MS=534.2 [M+H]⁺.

### Step 5: 3-chloro-5-{2-[(3S)-3-[(4-methanesulfonylphenoxy)methyl]piperazin-1-yl]ethyl}benzonitrile (Compound 189)

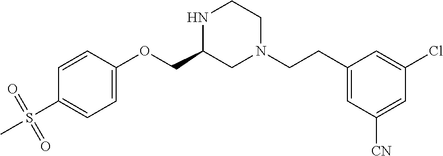 [SMILES: CS(=O)(=O)c1ccc(OC[C@@H]2CN(CCc3cc(Cl)cc(C#N)c3)CCN2)cc1]

To a solution of (S)-tert-butyl4-(3-chloro-5-cyanophenethyl)-2-((4-(methylsulfonyl)phenoxy) methyl)piperazine-1-carboxylate (600 mg, 1.12 mmol) in EtOAc (10 mL) was added HCl/EtOAc (4 M, 15 mL). The mixture was stirred at room temperature for 1 h and then concentrated under reduced pressure. The residue was purified by preparative reverse phase HPLC (Phenomenex C18 column, 20-50% MeCN: 10 mM NH₄HCO₃ in H₂O) to give 3-chloro-5-{2-[(3S)-3-[(4-methanesulfonylphenoxy)methyl]piperazin-1-yl]ethyl}benzonitrile (Compound 189). ¹H NMR (400 MHz, DMSO-d₆): δ 7.89-7.82 (m, 3H), 7.73-7.72 (m, 2H), 7.16 (d, J=8.8 Hz, 2H), 4.01-3.94 (m, 2H), 3.15 (s, 3H), 3.00-2.99 (m, 1H), 2.87-2.79 (m, 4H), 2.72-2.64 (m, 2H), 2.56-2.52 (m, 2H), 2.29 (s, 1H), 2.07-2.02 (m, 1H), 1.90 (t, J=9.6 Hz, 1H). MS=434.0 [M+H]⁺.

The following compound in Table 24 was prepared according to procedures similar to those described for Compound 189 using the appropriate starting materials.

TABLE 24

| | | | Exact Mass |
|---|---|---|---|
| No. | Structure | IUPAC Name | [M + H]⁺ |
| 190 | 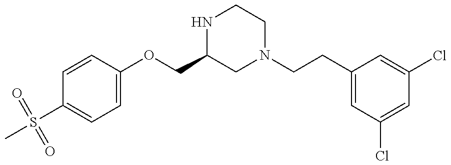 [SMILES: CS(=O)(=O)c1ccc(OC[C@@H]2CN(CCc3cc(Cl)cc(Cl)c3)CCN2)cc1] | (3S)-1-[2-(3,5- dichlorophenyl)ethyl]-3- [(4- methanesulfonylphenoxy) methyl]piperazine | Calc'd 443.1 Found 443.2 |

### Example 24

### (3S)-1-[2-(5-chloro-2-methoxyphenyl)ethyl]-3-[(4-methanesulfonylphenoxy)methyl]piperazine (Compound 191)

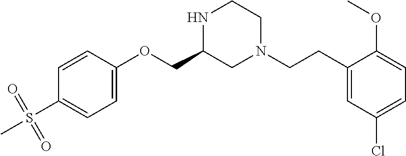 [SMILES: COc1ccc(Cl)cc1CCN1CCN[C@H](COc2ccc(S(C)(=O)=O)cc2)C1]

### Step 1: tert-butyl (S)-4-(5-chloro-2-methoxyphenethyl)-2-((4-(methylsulfonyl)phenoxy)methyl)piperazine-1-carboxylate

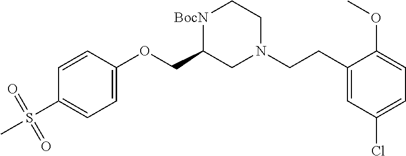 [SMILES: COc1ccc(Cl)cc1CCN1CCN(C(=O)OC(C)(C)C)[C@H](COc2ccc(S(C)(=O)=O)cc2)C1]

To a solution of Zn (221 mg, 3.37 mmol) and TMSCl (147 mg, 1.35 mmol) in CH₃CN (25 mL) was added two drops of 2-(bromomethyl)-4-chloro-1-methoxybenzene. The mixture was stirred at 40° C. for 1 h. Then HCHO (61 mg, 2.02 mmol), tert-butyl (S)-2-((4-(methylsulfonyl)phenoxy)methyl)piperazine-1-carboxylate (250 mg, 675 μmol) and 2-(bromomethyl)-4-chloro-1-methoxybenzene (397 mg, 1.69 mmol) were added to the mixture. The reaction was stirred at 40° C. for 16 h. The reaction was concentrated and then diluted with water (25 mL) and extracted with EtOAc (25 mL×3). The combined organic layers were washed with brine (20 mL), dried over Na₂SO₄, filtered and the filtrate was concentrated to give a residue. The residue was purified by preparative reverse phase HPLC (Phenomenex Gemini-NX, 50-70% MeCN:10 mM NH₄HCO₃ in H₂O) to give tert-butyl (S)-4-(5-chloro-2-methoxyphenethyl)-2-((4-(methylsulfonyl)phenoxy)methyl)piperazine-1-carboxylate. MS=539.2 [M+H]⁺.

### Step 2 (3S)-1-[2-(5-chloro-2-methoxyphenyl)ethyl]-3-[(4-methanesulfonylphenoxy)methyl]piperazine (Compound 191)

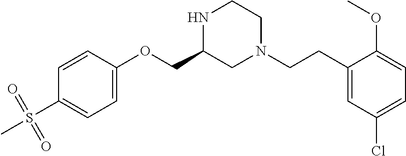 [SMILES: COc1ccc(Cl)cc1CCN1CCN[C@H](COc2ccc(S(C)(=O)=O)cc2)C1]

To a solution of tert-butyl (S)-4-(5-chloro-2-methoxyphenethyl)-2-((4-(methylsulfonyl)phenoxy)methyl)piperazine-1-carboxylate (60 mg, 111 μmol) in MeOH (1 mL) was added HCl/MeOH (4 M, 1 mL). The reaction mixture was stirred at room temperature for 2 h. The reaction mixture was then concentrated under reduced pressure to give (3S)-1-[2-(5-chloro-2-methoxyphenyl)ethyl]-3-[(4-methanesulfonylphenoxy)methyl]piperazine HCl salt (Compound 191). MS=439.2 [M+H]⁺, ¹H NMR (400 MHz, DMSO-d₆): δ 7.93-7.89 (m, 2H), 7.32-7.25 (m, 4H), 7.03 (d, J=8.8 Hz, 1H), 4.51-14.43 (i, 2H), 4.20-4.19 (i, 1H), 4.0-4.01 (m, 1H), 3.83-3.81 (m, 4H), 3.60-3.56 (m, 3H), 3.43-3.35 (m, 4H), 3.18 (s, 3H), 3.08-3.04 (m, 2H).

The following compounds in Table 25 were prepared according to procedures similar to those described for Compound 191 using the appropriate starting materials.

TABLE 25

| | | | Exact Mass |
|---|---|---|---|
| No. | Structure | IUPAC Name | [M + H]⁺ |
| 192 | 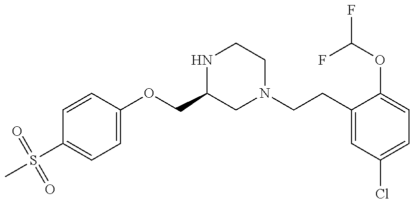 [SMILES: CS(=O)(=O)c1ccc(OC[C@@H]2CN(CCc3cc(Cl)ccc3OC(F)F)CCN2)cc1] | (3S)-1-{2-[5-chloro-2- (difluoromethoxy)phenyl]eth- yl}-3-[(4- methanesulfonylphenoxy) methyl]piperazine | Calc'd 475.1 Found 475.2 |
| 193 | 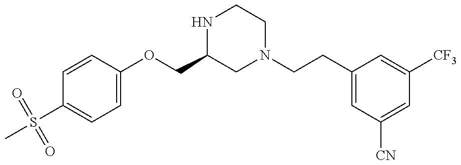 [SMILES: CS(=O)(=O)c1ccc(OC[C@@H]2CN(CCc3cc(C#N)cc(C(F)(F)F)c3)CCN2)cc1] | 3-{2-[(3S)-3-[(4- methanesulfonylphenoxy) methyl]piperazin-1- yl]ethyl}-5- (trifluoromethyl)benzonitrile | Calc'd 468.2 Found 468.2 |
| 194 | 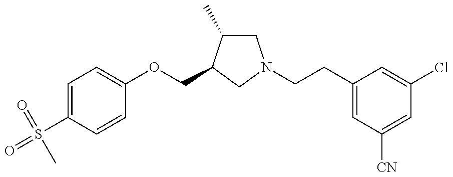 [SMILES: C[C@@H]1CN(CCc2cc(Cl)cc(C#N)c2)C[C@H]1COc1ccc(S(C)(=O)=O)cc1] | 3-chloro-5-{2-[(3S,4S)-3- [(4- methanesulfonylphenoxy) methyl]-4- methylpyrrolidin-1- yl]ethyl}benzonnitrile | Calc'd 433.1 Found 433.1 |
| 195 | 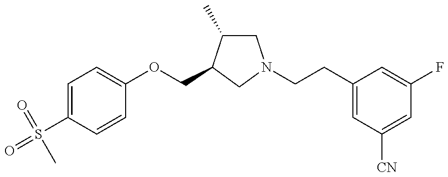 [SMILES: C[C@@H]1CN(CCc2cc(F)cc(C#N)c2)C[C@H]1COc1ccc(S(C)(=O)=O)cc1] | 3-fluoro-5-{2-[(3S,4S)-3- [(4- methanesulfonylphenoxy) methyl]-4- methylpyrrolidin-1- yl]ethyl}benzonitrile | Calc'd 417.2 Found 417.2 |
| 196 | 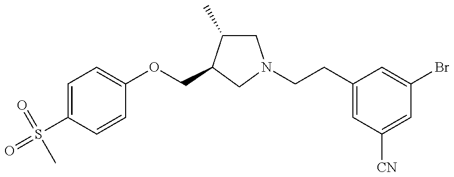 [SMILES: C[C@@H]1CN(CCc2cc(Br)cc(C#N)c2)C[C@H]1COc1ccc(S(C)(=O)=O)cc1] | 3-bromo-5-{2-[(3S,4S)-3- [(4- methanesulfonylphenoxy) methyl]-4- methylpyrrolidin-1- yl]ethyl}benzonitrile | Calc'd 477.1 Found 477.1 |
| 181 | 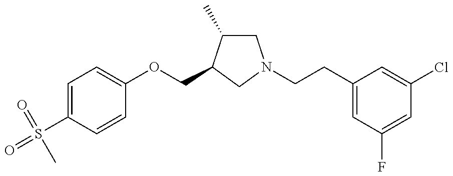 [SMILES: C[C@@H]1CN(CCc2cc(F)cc(Cl)c2)C[C@H]1COc1ccc(S(C)(=O)=O)cc1] | (3S,4S)-1-[2-(3-chloro-5- fluorophenyl)ethyl]-3-[(4- methanesulfonylphenoxy) methyl]-4- methylpyrrolidine | Calc'd 426.1 Found 426.1 |

### Example 25

### (3S)-1-[2-(5-chloro-2-methylphenyl)ethyl]-3-[(4-methanesulfonylphenoxy)methyl]piperazine (Compound 197)

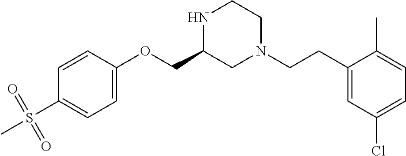 [SMILES: Cc1ccc(Cl)cc1CCN1CCN[C@H](COc2ccc(S(C)(=O)=O)cc2)C1]

### Step 1: (S)-tert-butyl 2-((4-(methylsulfonyl)phenoxy)methyl)piperazine-1-carboxylate

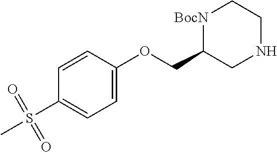 [SMILES: CC(C)(C)OC(=O)N1CCNC[C@H]1COc1ccc(S(C)(=O)=O)cc1]

To a suspension of Pd/C (100 mg, 10% by weight in mineral oil) in MeOH (15 mL) was added (S)-4-benzyl 1-tert-butyl 2-((4-(methylsulfonyl)phenoxy)methyl)piperazine-1,4-dicarboxylate (500 mg, 991 mmol). The suspension was degassed under vacuum and purged with H₂ three times. The mixture was then stirred under H₂ (15 psi) at room temperature for 4 h. The mixture was filtered through a pad of Celite, and the filter cake was rinsed with MeOH (8 mL×3). The combined filtrate was concentrated under reduced pressure. The residue was dissolved in EtOAc (30 mL) and saturated citric acid solution (30 mL). The organic layer was separated, and the aqueous layer was extracted with EtOAc (15 mL). The aqueous layer was adjusted to pH>7 by addition of solid NaHCO₃, and then extracted with EtOAc (20 mL×2). The combined organic layers were dried over Na₂SO₄, filtered and concentrated under reduced pressure to give (S)-tert-butyl 2-((4-(methylsulfonyl)phenoxy)methyl)piperazine-1-carboxylate, which was taken to the next step without further purification. MS=271.1 [M-C₅H₉O₂+H]⁺.

### Step 2: (S)-tert-butyl 4-(2-bromo-5-chlorophenethyl)-2-((4-(methylsulfonyl)phenoxy)methyl)piperazine-1-carboxylate

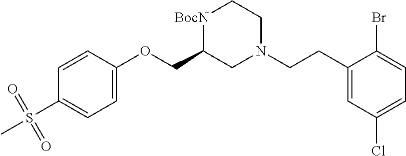 [SMILES: CC(C)(C)OC(=O)N1CCN(CCc2cc(Cl)ccc2Br)C[C@H]1COc1ccc(S(C)(=O)=O)cc1]

To a solution of (S)-tert-butyl 2-((4-(methylsulfonyl)phenoxy)methyl)piperazine-1-carboxylate (170 mg, 459 μmol) and 2-(2-bromo-5-chloro-phenyl)acetaldehyde (107 mg, 459 μmol) in DCM (5 mL) at room temperature was added AcOH (459 μmol, 26 uL) followed by NaBH(OAc)₃ (146 mg, 688 μmol). The mixture was then stirred for 1 h. To the mixture was added saturated aqueous NaHCO₃ (15 mL) and DCM (10 mL). The organic layer was separated, and the aqueous layer was extracted with DCM (10 mL). The combined organic layers were dried over Na₂SO₄, filtered and concentrated under reduced pressure. The residue was purified by flash silica gel chromatography (Biotage 12 g cartridge, 0-40% EtOAc:Hexane) to give (S)-tert-butyl 4-(2-bromo-5-chlorophenethyl)-2-((4-(methylsulfonyl)phenoxy)methyl)piperazine-1-carboxylate. MS=587.2/589.2 [M+H]⁺

### Step 3: (S)-tert-butyl 4-(5-chloro-2-methylphenethyl)-2-((4-(methylsulfonyl)phenoxy)methyl)piperazine-1-carboxylate

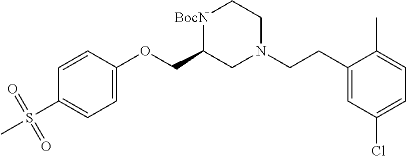 [SMILES: Cc1ccc(Cl)cc1CCN1CCN(C(=O)OC(C)(C)C)[C@H](COc2ccc(S(C)(=O)=O)cc2)C1]

To a mixture of (S)-tert-butyl 4-(2-bromo-5-chlorophenethyl)-2-((4-(methylsulfonyl)phenoxy)methyl)piperazine-1-carboxylate (130 mg, 221 μmol), methylboronic acid (27 mg, 442 μmol) and K₂CO₃ (61 mg, 442 μmol) in 1,4-dioxane (4 mL) and H₂O (0.4 mL) was added Pd(dppf)Cl₂·CH₂Cl₂ (18 mg, 22.1 μmol) at room temperature under N₂. The resulting mixture was then stirred at 100° C. for 16 h under N₂. The mixture was cooled to room temperature and concentrated under reduced pressure. The residue was purified by flash silica gel chromatography (Biotage 12 g cartridge, 0-100% EtOAc:Hexane) to give (S)-tert-butyl 4-(5-chloro-2-methylphenethyl)-2-((4-(methylsulfonyl)phenoxy)methyl)piperazine-1-carboxylate, which was taken to the next step without further purification. MS=523.3 [M+H]⁺.

### Step 4: (3S)-1-[2-(5-chloro-2-methylphenyl)ethyl]-3-[(4-methanesulfonylphenoxy)methyl]piperazine (Compound 197)

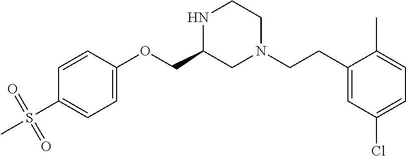 [SMILES: Cc1ccc(Cl)cc1CCN1CCN[C@H](COc2ccc(S(C)(=O)=O)cc2)C1]

A mixture of (S)-tert-butyl 4-(5-chloro-2-methylphenethyl)-2-((4-(methylsulfonyl)phenoxy)methyl)piperazine-1-carboxylate (120 mg, 229 μmol) in HCl/EtOAc (4 M, 8 mL) was stirred at room temperature for 0.5 h. The mixture was concentrated under reduced pressure. The solid residue was washed with EtOAc (8 mL), and then purified by preparative reverse phase HPLC (Waters Xbridge BEH C18 column, 30-60% MeCN:10 mM NH₄HCO₃ in H₂O) to give (3S)-1-[2-(5-chloro-2-methylphenyl)ethyl]-3-[(4-methanesulfonylphenoxy)methyl]piperazine (Compound 197). ¹H NMR (400 MHz, DMSO-d₆): δ 7.83 (d, J=9.2 Hz, 2H), 7.23 (s, 1H), 7.18-7.13 (m, 4H), 4.02-3.96 (m, 2H), 3.15 (s, 3H), 3.04-2.95 (m, 1H), 2.92-2.86 (m, 2H), 2.79-2.64 (m, 4H), 2.47-2.43 (m, 3H), 2.32 (s, 3H), 2.10-2.00 (m, 1H), 1.92-1.87 (m, 1H). MS=423.3 [M+H]⁺.

### Example 26

### 3-{2-[(3S,4S)-3-[(4-methanesulfonylphenoxy)methyl]-4-methylpyrrolidin-1-yl]ethyl}-5-methylbenzonitrile (Compound 198)

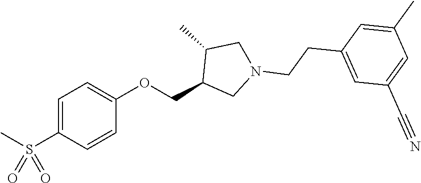 [SMILES: Cc1cc(C#N)cc(CCN2C[C@@H](COc3ccc(S(C)(=O)=O)cc3)[C@H](C)C2)c1]

### Step 1: 3-{2-[(3S,4S)-3-[(4-methanesulfonylphenoxy)methyl]-4-methylpyrrolidin-1-yl]ethyl}-5-methylbenzonitrile (Compound 198)

A vial was charged with 3-bromo-5-{2-[(3S,4S)-3-[(4-methanesulfonylphenoxy)methyl]-4-methylpyrrolidin-1-yl]ethyl}benzonitrile (100 mg, 0.209 mmol), methylboronic acid (190 mg, 0.314 mmol), K₂CO₃ (58 mg, 0.419 mmol), tetrakis(triphenylphosphine)palladium(0) (10 mg, 0.008 mmol), Toluene (1.0 mL) and H₂O (0.21 mL). The mixture was then sparged with nitrogen gas for 1 minute and then heated to 100° C. for 16 h. Reaction was partitioned with H₂O and EtOAc and extracted 3 times. The combined organics layers were dried over Na₂SO₄, filtered and concentrated. The crude oil was purified by prep-HPLC (5-50% MeCN in H₂O with 0.1% formic acid modifier) to give 3-{2-[(3S,4S)-3-(4-methanesulfonylphenoxymethyl)-4-methylpyrrolidin-1-yl]ethyl}-5-methylbenzonitrile (Compound 198). ¹H NMR (500 MHz, CDCl₃): δ 7.82-7.77 (m, 2H), 7.27-7.17 (m, 3H), 6.97-6.92 (m, 2H), 5.00-3.49 (m, 3H), 4.02-3.93 (m, 2H), 3.15 (t, J=8.5 Hz, 1H), 2.96 (d, J=1.2 Hz, 3H), 2.92 (t, J=7.4 Hz, 2H), 2.85 (d, J=5.5 Hz, 2H), 2.84-2.80 (m, 1H), 2.80-2.73 (m, 1H), 2.34 (t, J=9.0 Hz, 1H), 2.28-2.21 (m, 1H), 2.18-2.10 (m, 1H), 1.11 (d, J=6.6 Hz, 3H). MS=413.2 [M+H]⁺.

### Example 27

### rac-cis or trans-3-chloro-5-{2-[4-[(4-methanesulfonylphenoxy)methyl]-2-methylpyrrolidin-1-yl]ethyl}benzonitrile (Compound 199)

### 3-chloro-5-(2-((2S,4R or 2R,4S or 2R,4R or 2S,4S)-2-methyl-4-((4-(methylsulfonyl)phenoxy)methyl)pyrrolidin-1-yl)ethyl)benzonitrile (Compound 200)

### 3-chloro-5-(2-((2R,4S or 2S,4R or 2R,4R or 2S,4S)-2-methyl-4-((4-(methylsulfonyl)phenoxy)methyl)pyrrolidin-1-yl)ethyl)benzonitrile (Compound 201)

### rac-trans or cis-3-chloro-5-{2-[4-[(4-methanesulfonylphenoxy)methyl]-2-methylpyrrolidin-1-yl]ethyl}benzonitrile (Compound 202)

### 3-chloro-5-(2-((2R,4S or 2S,4R or 2R,4R or 2S,4S)-2-methyl-4-((4-(methylsulfonyl)phenoxy)methyl)pyrrolidin-1-yl)ethyl)benzonitrile (Compound 203)

### 3-chloro-5-(2-((2R,4S or 2S,4R or 2S,4S or 2R.4R)-2-methyl-4-((4-(methylsulfonyl)phenoxy)methyl)pyrrolidin-1-yl)ethyl)benzonitrile (Compound 204)

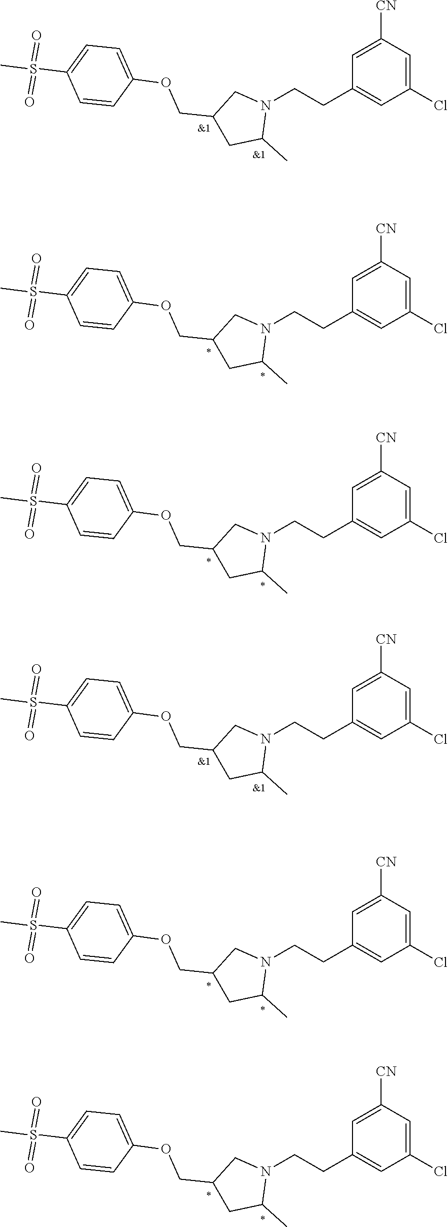 [SMILES: CC1CC(COc2ccc(S(C)(=O)=O)cc2)CN1CCc1cc(Cl)cc(C#N)c1 ; CC1CC(COc2ccc(S(C)(=O)=O)cc2)CN1CCc1cc(Cl)cc(C#N)c1 ; CC1CC(COc2ccc(S(C)(=O)=O)cc2)CN1CCc1cc(Cl)cc(C#N)c1 ; CC1CC(COc2ccc(S(C)(=O)=O)cc2)CN1CCc1cc(Cl)cc(C#N)c1 ; CC1CC(COc2ccc(S(C)(=O)=O)cc2)CN1CCc1cc(Cl)cc(C#N)c1 ; CC1CC(COc2ccc(S(C)(=O)=O)cc2)CN1CCc1cc(Cl)cc(C#N)c1]

### Step 1: rac-cis or trans-3-chloro-5-{2-[4-[(4-methanesulfonylphenoxy)methyl]-2-methylpyrrolidin-1-yl]ethyl}benzonitrile (Compound 199) and rac-trans or cis-3-chloro-5-{2-[4-[(4-methanesulfonylphenoxy)methyl]-2-methylpyrrolidin-1-yl]ethyl}benzonitrile (Compound 202)

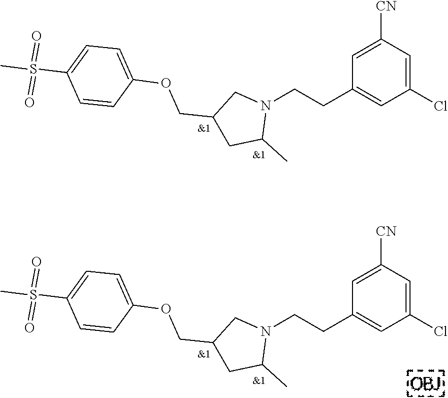 [SMILES: CC1CC(COc2ccc(S(C)(=O)=O)cc2)CN1CCc1cc(Cl)cc(C#N)c1 ; CC1CC(COc2ccc(S(C)(=O)=O)cc2)CN1CCc1cc(Cl)cc(C#N)c1]

To a mixture of Zn (23 mg, 356 μmol) in MeCN (2 mL) were added three drops of 3-(bromomethyl)-5-chloro-benzonitrile in 0.1 mL of MeCN and TMSCl (200 μmol, 25 uL). The mixture was stirred at 50° C. for 20 min. Then 3-(bromomethyl)-5-chloro-benzonitrile (39 mg, 167 μmol), 2-methyl-4-[(4-methylsulfonylphenoxy)methyl]pyrrolidine (30 mg, 111 μmol) and paraformaldehyde (20 mg, 222 μmol) were added to the mixture successively. The resulting mixture was stirred at room temperature for 1 h. To the mixture was added H₂O (10 mL), then the mixture was extracted with EtOAc (10 mL×3). The combined organic layers were dried over Na₂SO₄, filtered and the filtrate was concentrated under reduced pressure. The residue was purified by preparative reverse phase HPLC (Waters Xbridge BEH C18 column, 40-65% MeCN:10 mM NH₄HCO₃ in H₂O). The first eluting isomer (Compound 199): ¹H NMR (400 MHz, DMSO-d₆): δ 7.84-7.81 (m, 3H), 7.76-7.74 (m, 2H), 7.17-7.14 (m, 2H), 4.04-3.93 (m, 2H), 3.35-3.34 (m, 1H), 3.15 (s, 3H), 3.01-2.98 (m, 1H), 2.80-2.70 (m, 2H), 2.46-2.45 (m, 2H), 2.33-2.30 (m, 1H), 2.03 (t, J=7.2 Hz, 1H), 1.72-1.71 (m, 1H), 1.53-1.52 (m, 1H), 0.98 (d, J=6.0 Hz, 3H). MS=433.2 [M+H]⁺. The second eluting isomer (Compound 202): ¹H NMR (400 MHz, DMSO-d₆): δ 7.84-7.80 (m, 3H), 7.72-7.70 (m, 2H), 7.14 (d, J=8.8 Hz, 2H), 3.89-3.87 (m, 2H), 3.15 (s, 3H), 3.07-3.04 (m, 1H), 2.96-2.95 (m, 1H), 2.78-2.76 (m, 2H), 2.52-2.51 (m, 1H), 2.33-2.27 (m, 3H), 2.11-2.07 (m, 1H), 1.06-1.01 (m, 1H), 0.96 (d, J=6.0 Hz, 3H). MS=433.2 [M+H]⁺.

### Step 2: 3-chloro-5-{2-[(2R,4S or 2S,4R or 2R,4R or 2S,4S)-4-[(4-methanesulfonylphenoxy)methyl]-2-methylpyrrolidin-1-yl]ethyl}benzonitrile (Compound 200) and 3-chloro-5-{2-[(2S,4R or 2R,4S or 2R,4R or 2S,4S)-4-[(4-methanesulfonylphenoxy)methyl]-2-methylpyrrolidin-1-yl]ethyl}benzonitrile (Compound 201)

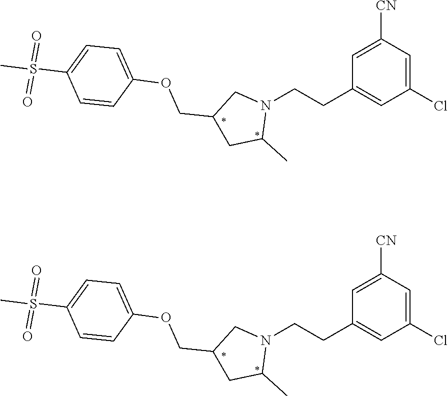 [SMILES: CC1CC(COc2ccc(S(C)(=O)=O)cc2)CN1CCc1cc(Cl)cc(C#N)c1 ; CC1CC(COc2ccc(S(C)(=O)=O)cc2)CN1CCc1cc(Cl)cc(C#N)c1]

The first eluting isomer from Step 1 was further purified by preparative chiral SFC (CHIRALPAK AD, 60% ethanol with 0.1% NH₄OH in CO₂). The first eluting isomer of the title compound (Compound 200): ¹H NMR (400 MHz, DMSO-d₆): δ 7.8-7.82 (m, 3H), 7.76-7.74 (m, 2H), 7.15 (d, J=8.8 Hz, 2H), 4.04-3.95 (m, 2H), 3.35-3.34 (m, 1H), 3.15 (s, 3H), 3.01-2.98 (m, 1H), 2.83-2.75 (m, 2H), 2.52-2.50 (m, 2H), 2.31-2.29 (m, 1H), 2.03 (t, J=7.2 Hz, 1H), 1.72-1.71 (m, 1H), 1.53-1.50 (m, 1H), 0.98 (d, J=6.0 Hz, 3H). MS=433.2 [M+H]⁺

The second eluting isomer of the title compound (Compound 201). ¹H NMR (400 MHz, DMSO-d₆): δ 7.84-7.82 (m, 3H), 7.76-7.74 (m, 2H), 7.15 (d, J=8.8 Hz, 2H), 4.04-3.95 (m, 2H), 3.35-3.32 (m, 1H), 3.15 (s, 3H), 3.01-2.98 (m, 1H), 2.83-2.76 (m, 2H), 2.52-2.50 (m, 2H), 2.31-2.29 (m, 1H), 2.05-2.03 (m, 1H), 1.72-1.71 (m, 1H), 1.53-1.50 (m, 1H), 0.98 (d, J=5.6 Hz, 3H). MS=433.2 [M+H]⁺.

### Step 3: 3-chloro-5-(2-((2R,4S or 2S,4R or 2R,4R or 2S,4S)-2-methyl-4-((4-(methylsulfonyl)phenoxy)methyl)pyrrolidin-1-yl)ethyl)benzonitrile (Compound 203) and 3-chloro-5-(2-((2R,4S or 2S,4R or 2S,4S or 2R.4R)-2-methyl-4-((4-(methylsulfonyl)phenoxy)methyl)pyrrolidin-1-yl)ethyl)benzonitrile (Compound 204)

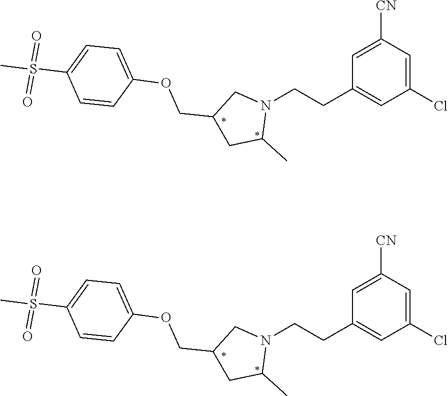 [SMILES: CC1CC(COc2ccc(S(C)(=O)=O)cc2)CN1CCc1cc(Cl)cc(C#N)c1 ; CC1CC(COc2ccc(S(C)(=O)=O)cc2)CN1CCc1cc(Cl)cc(C#N)c1]

The second eluting isomer from Step 1 (150 mg, 346 μmol) was further purified by preparative chiral SFC (CHIRALPAK AD, 42% ethanol with 0.1% NH₄OH in CO₂). The first eluting isomer of the title compound (Compound 203): ¹H NMR (DMSO-d₆, 400 MHz): δ 7.84-7.81 (m, 3H), 7.73-7.70 (m, 2H), 7.14 (d, J=8.8 Hz, 2H), 3.90-3.88 (m, 2H), 3.15 (s, 3H), 3.07-3.04 (m, 1H), 2.97-2.96 (m, 1H), 2.79-2.73 (m, 2H), 2.52-2.50 (m, 1H), 2.33-2.28 (m, 3H), 2.15-2.13 (m, 1H), 1.07-1.01 (m, 1H), 0.96 (d, J=5.6 Hz, 3H). MS=433.2 [M+H]⁺.

The second eluting isomer of the title compound (Compound 204): ¹H NMR (400 MHz, DMSO-d₆): δ 7.86-7.81 (m, 3H), 7.73-7.70 (m, 2H), 7.17-7.13 (m, 2H), 3.90-3.88 (m, 2H), 3.15 (s, 3H), 3.05-3.04 (m, 1H), 2.99-2.98 (m, 1H), 2.81-2.76 (m, 2H), 2.59-2.57 (m, 1H), 2.33-2.27 (m, 3H), 2.15-2.14 (m, 1H), 1.07-1.01 (m, 1H), 0.96 (d, J=5.6 Hz, 3H). MS=433.2 [M+H]⁺.

### Example 28

### rac-cis-1-[2-(3-chlorophenyl)ethyl]-4-[(4-methanesulfonylphenoxy)methyl]-3-methylpyrrolidin-3-ol (Compound 205)

### (3R,4R or 3S,4S)-1-[2-(3-chlorophenyl)ethyl]-4-[(4-methanesulfonylphenoxy)methyl]-3-methylpyrrolidin-3-ol (Compound 206)

### (3S,4S or 3R,4R)-1-[2-(3-chlorophenyl)ethyl]-4-[(4-methanesulfonylphenoxy)methyl]-3-methylpyrrolidin-3-ol (Compound 207)

### rac-trans-1-[2-(3-chlorophenyl)ethyl]-4-[(4-methanesulfonylphenoxy)methyl]-3-methylpyrrolidin-3-ol (Compound 208)

### (3R,4S or 3S,4R)-1-[2-(3-chlorophenyl)ethyl]-4-[(4-methanesulfonylphenoxy)methyl]-3-methylpyrrolidin-3-ol pyrrolidine-3-ol (Compound 209)

### (3S,4R or 3R,4S)-1-[2-(3-chlorophenyl)ethyl]-4-[(4-methanesulfonylphenoxy)methyl]-3-methylpyrrolidin-3-ol (Compound 210)

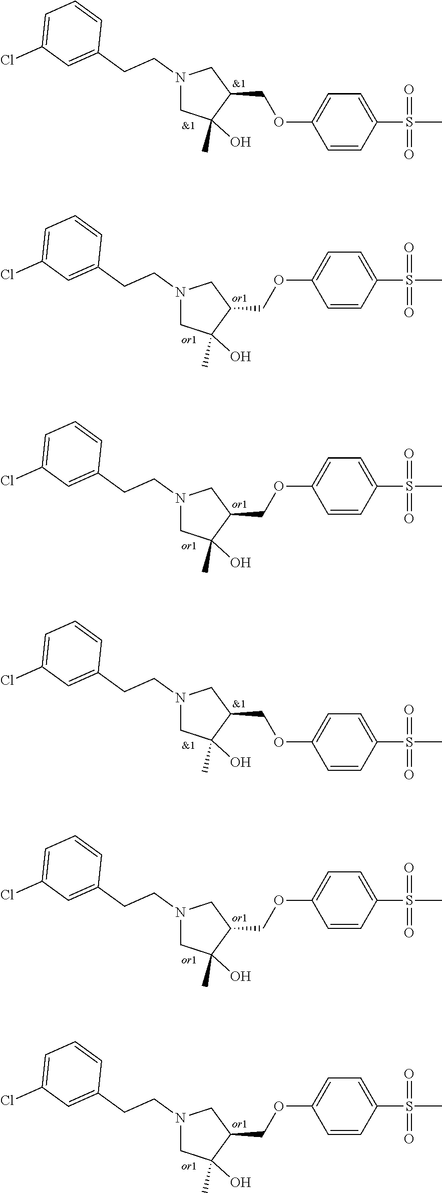 [SMILES: C[C@@]1(O)CN(CCc2cccc(Cl)c2)C[C@H]1COc1ccc(S(C)(=O)=O)cc1 ; C[C@]1(O)CN(CCc2cccc(Cl)c2)C[C@@H]1COc1ccc(S(C)(=O)=O)cc1 ; C[C@@]1(O)CN(CCc2cccc(Cl)c2)C[C@H]1COc1ccc(S(C)(=O)=O)cc1 ; C[C@]1(O)CN(CCc2cccc(Cl)c2)C[C@H]1COc1ccc(S(C)(=O)=O)cc1 ; C[C@@]1(O)CN(CCc2cccc(Cl)c2)C[C@@H]1COc1ccc(S(C)(=O)=O)cc1 ; C[C@]1(O)CN(CCc2cccc(Cl)c2)C[C@H]1COc1ccc(S(C)(=O)=O)cc1]

### Step 1: tert-butyl 3-hydroxy-4-(hydroxymethyl)pyrrolidine-1-carboxylate

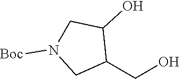 [SMILES: CC(C)(C)OC(=O)N1CC(O)C(CO)C1]

To a solution of 1-ter-butyl 3-ethyl 4-oxopyrrolidine-1,3-dicarboxylate (10.0 g, 38.9 mmol) in EtOH (200 mL) at 0° C. was slowly added NaBH₄ (14.7 g, 389 mmol). The mixture was stirred at room temperature for 15 h. The reaction mixture was quenched with ice water (300 mL), and then extracted with EtOAc (100 mL×3). The combined organic layers were dried over Na₂SO₄, filtered and concentrated under reduced pressure. The residue was purified by flash silica gel chromatography (Biotage 80 g cartridge, 0-100% EtOAc:Hexane) to give tert-butyl 3-hydroxy-4-(hydroxymethyl)pyrrolidine-1-carboxylate. MS=162.1 [M-C₄H₈+H]⁺.

### Step 2: tert-butyl 3-hydroxy-4-((4-(methylsulfonyl)phenoxy)methyl)pyrrolidine-1-carboxylate

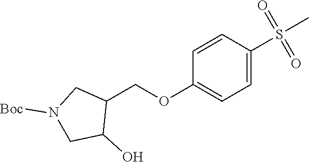 [SMILES: CC(C)(C)OC(=O)N1CC(O)C(COc2ccc(S(C)(=O)=O)cc2)C1]

To a mixture of 1-fluoro-4-methylsulfonyl-benzene (5.21 g, 29.92 mmol) and tert-butyl 3-hydroxy-4-(hydroxymethyl)pyrrolidine-1-carboxylate (6.50 g, 29.9 mmol) in DMF (50 mL) was added K₂CO₃ (8.27 g, 59.84 mmol). The mixture was stirred at 100° C. for 60 h. The reaction mixture was diluted with water (150 mL), and then extracted with EtOAc (50 mL×3). The combined organic layers were dried over Na₂SO₄, filtered and concentrated under reduced pressure. The residue was purified by flash silica gel chromatography (Biotage 80 g cartridge, 0-50% EtOAc:Hexane) to give tert-butyl 3-hydroxy-4-((4-(methylsulfonyl)phenoxy)methyl)pyrrolidine-1-carboxylate. MS=316.1 [M-C₄H₈+H]⁺.

### Step 3: tert-butyl 3-((4-(methylsulfonyl)phenoxy)methyl)-4-oxopyrrolidine-1-carboxylate

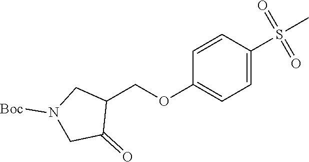 [SMILES: CC(C)(C)OC(=O)N1CC(=O)C(COc2ccc(S(C)(=O)=O)cc2)C1]

To a solution of tert-butyl 3-hydroxy-4-((4-(methylsulfonyl)phenoxy)methyl)pyrrolidine-1-carboxylate (4.90 g, 13.2 mmol) in DCM (50 mL) at 0° C. was added DMP (6.71 g, 15.8 mmol). The mixture was stirred at room temperature for 15 h. The reaction mixture was cooled to 0° C., quenched with saturated aqueous Na₂SO₃ (100 mL), concentrated, then extracted with EtOAc (50 mL×5). The combined organic layers were dried over Na₂SO₄, filtered and concentrated under reduced pressure. The residue was purified by flash silica gel chromatography (Biotage 40 g cartridge, 0-100% EtOAc:Hexane) to give tert-butyl 3-((4-(methylsulfonyl)phenoxy)methyl)-4-oxopyrrolidine-1-carboxylate. MS=314.1 [M-C₄H₈+H]⁺.

### Step 4: rac-cis-tert-butyl 3-hydroxy-3-methyl-4-((4-(methylsulfonyl)phenoxy)methyl)pyrrolidine-1-carboxylate and rac-trans-tert-butyl 3-hydroxy-3-methyl-4-((4-(methylsulfonyl)phenoxy)methyl)pyrrolidine-1-carboxylate

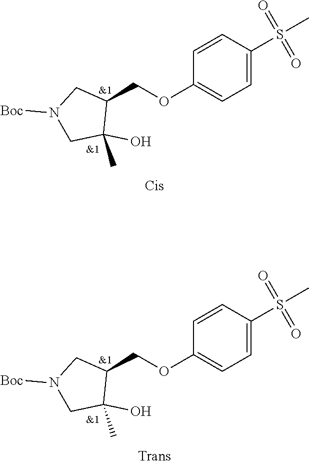 [SMILES: CC(C)(C)OC(=O)N1C[C@@H](COc2ccc(S(C)(=O)=O)cc2)[C@](C)(O)C1 ; CC(C)(C)OC(=O)N1C[C@@H](COc2ccc(S(C)(=O)=O)cc2)[C@@](C)(O)C1]

A solution of tert-butyl 3-((4-(methylsulfonyl)phenoxy)methyl)-4-oxopyrrolidine-1-carboxylate (370 mg, 1.00 mmol) in THF (2 mL) was degassed and purged with N₂ three times. The mixture was cooled to 0° C. and MeMgBr (3 M in 2-MeTHF, 667 uL, 2.0 mmol) was added dropwise. The mixture was stirred at room temperature for 3 h under N₂ atmosphere. The reaction mixture was cooled to 0° C., quenched with water (10 mL), and extracted with EtOAc (15 mL×3). The combined organic layers were dried over Na₂SO₄, filtered and concentrated under reduced pressure. The residue was purified by preparative reverse phase HPLC (Phenomenex Gemini-NX, 20-50% MeCN: 10 mM NH₄HCO₃ in H₂O) to give rac-cis-tert-butyl 3-hydroxy-3-methyl-4-((4-(methylsulfonyl)phenoxy)methyl)pyrrolidine-1-carboxylate (the first eluting isomer in HPLC). MS=330.1 [M-C₄H₈+H]⁺. And rac-trans-tert-butyl 3-hydroxy-3-methyl-4-((4-(methylsulfonyl)phenoxy)methyl)pyrrolidine-1-carboxylate (the second eluting isomer in HPLC). MS=330.1 [M-C₄H₈+H]⁺.

### Step 5: rac-cis-3-methyl-4-((4-(methylsulfonyl)phenoxy)methyl)pyrrolidin-3-ol

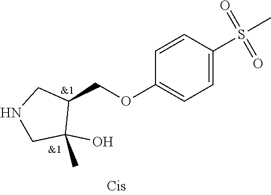 [SMILES: C[C@@]1(O)CNC[C@H]1COc1ccc(S(C)(=O)=O)cc1]

To a solution of the 1st eluting isomer from Step 4 (40.0 mg, 104 μmol) in DCM (0.3 mL) at 0° C. was added TFA (154 mg, 1.35 mmol). The mixture was stirred at room temperature for 1.5 h. The reaction mixture was concentrated under reduced pressure to give rac-cis-3-methyl-4-((4-(methylsulfonyl)phenoxy)methyl)pyrrolidin-3-ol, which was used without further purification. MS=286.2 [M+H]⁺.

### Step 6: rac-cis-1-[2-(3-chlorophenyl)ethyl]-4-[(4-methanesulfonylphenoxy)methyl]-3-methylpyrrolidin-3-ol (Compound 205)

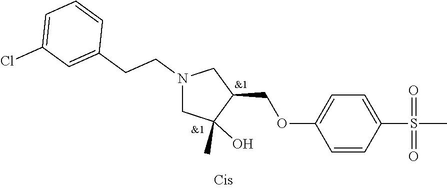 [SMILES: C[C@@]1(O)CN(CCc2cccc(Cl)c2)C[C@H]1COc1ccc(S(C)(=O)=O)cc1]

To a solution of the residue from Step 5 (29.0 mg, 102 μmol) in MeOH (0.3 mL) were added TEA (10.3 mg, 102 μmol), HOAc (5.8 uL, 102 μmol,) and 2-(3-chlorophenyl)acetaldehyde (23.6 mg, 152 μmol). After stirred at room temperature for 1 h, NaBH₃CN (9.58 mg, 152 μmol) was added. The mixture was stirred at room temperature for 2 h, then was cooled to 0° C. and quenched by addition of H₂O (0.2 mL). The residue was purified by preparative reverse phase HPLC (Waters Xbridge Prep OBD C18 column, 30-60% MeCN:10 mM NH₄HCO₃ in H₂O) to give rac-cis-1-[2-(3-chlorophenyl)ethyl]-4-[(4-methanesulfonylphenoxy)methyl]-3-methylpyrrolidin-3-ol (Compound 205). ¹H NMR (400 MHz DMSO-d₆): δ 7.83 (d, J=8.4 Hz, 2H), 7.31-7.16 (m, 6H), 4.77 (s, 1H), 4.18-4.14 (m, 1H), 3.94 (t, J=8.8 Hz, 1H), 3.15 (s, 3H), 3.05-2.95 (m, 1H), 2.73-2.61 (m, 6H), 2.44-2.42 (m, 2H), 1.21 (s, 3H). MS=424.2 [M+H]⁺.

### Step 7 (3R,4R or 3S,4S)-1-[2-(3-chlorophenyl)ethyl]-4-[(4-methanesulfonylphenoxy)methyl]-3-methylpyrrolidin-3-ol (Compound 206) and (3S,4S or 3R,4R)-1-(3-chlorophenethyl)-3-methyl-4-((4-(methylsulfonyl)phenoxy)methyl)pyrrolidin-3-ol (Compound 207)

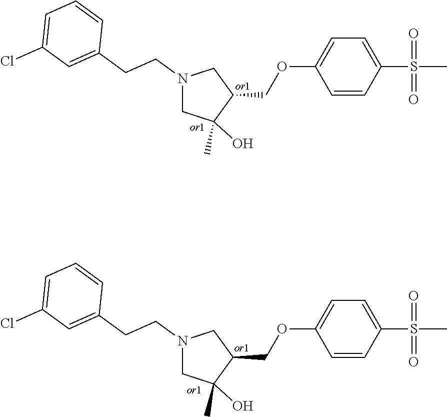 [SMILES: C[C@]1(O)CN(CCc2cccc(Cl)c2)C[C@@H]1COc1ccc(S(C)(=O)=O)cc1 ; C[C@@]1(O)CN(CCc2cccc(Cl)c2)C[C@H]1COc1ccc(S(C)(=O)=O)cc1]

The material obtained from Step 6 was further purified by chiral SFC (CHIRALPAK IG, 55% isopropanol with 0.1% NH₄OH in CO₂) The first eluting isomer of the title compound (Compound 206): ¹H NMR (400 MHz, DMSO-d₆): δ 7.83 (d, J=8.8 Hz, 2H), 7.31-7.24 (m, 4H), 7.21-7.16 (m, 2H), 4.79 (s, 1H), 4.18-4.14 (m, 1H), 3.95 (t, J=8.8 Hz, 1H), 3.15 (s, 3H), 3.05-2.95 (m, 1H), 2.71-2.61 (m, 6H), 2.45-2.35 (m, 2H), 1.21 (s, 3H). MS=424.2 [M+H]⁺. The second eluting isomer of the title compound (Compound 207): ¹H NMR (400 MHz, DMSO-d₆): δ 7.84 (d, J=8.8 Hz, 2H), 7.33-7.26 (m, 4H), 7.21-7.17 (m, 2H), 4.82 (s, 1H), 4.18-4.14 (m, 1H), 4.02-3.97 (m, 1H), 3.16 (s, 3H), 3.05-2.95 (m, 1H), 2.90-2.74 (m, 4H), 2.64-2.54 (m, 2H), 2.46-2.87 (m, 2H), 1.23 (s, 3H). MS=424.2 [M+H]⁺.

### Step 8: rac-trans-3-methyl-4-((4-(methylsulfonyl)phenoxy)methyl)pyrrolidin-3-ol

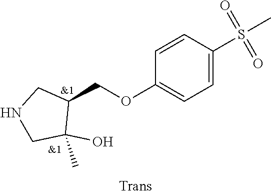 [SMILES: C[C@]1(O)CNC[C@H]1COc1ccc(S(C)(=O)=O)cc1]

To a solution of the 2^{nd} eluting isomer from Step 4 (70.0 mg, 182 μmol) in DCM (0.6 mL) at 0° C. was added TFA (308 mg, 2.70 mmol). The mixture was stirred at room temperature for 1.5 h, then was concentrated under reduced pressure to give rac-trans-3-methyl-4-((4-(methylsulfonyl)phenoxy)methyl)pyrrolidin-3-ol. MS=286.1 [M+H]⁺.

### Step 9: rac-trans-1-[2-(3-chlorophenyl)ethyl]-4-[(4-methanesulfonylphenoxy)methyl]-3-methylpyrrolidin-3-ol (Compound 208)

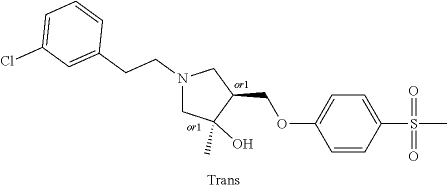 [SMILES: C[C@]1(O)CN(CCc2cccc(Cl)c2)C[C@H]1COc1ccc(S(C)(=O)=O)cc1]

To a solution of the residue from Step 8 (51.0 mg, 179 μmol) in MeOH (0.5 mL) were added TEA (25 uL, 178.72 μmol), HOAc (10 uL, 178 μmol) and 2-(3-chlorophenyl)acetaldehyde (41.4 mg, 268 μmol). After stirred at room temperature for 1 h, NaBH₃CN (16.9 mg, 268 μmol) was added, and the mixture was stirred at for 2 h. The reaction mixture was cooled to 0° C. and quenched by addition H₂O (0.2 mL). The residue was purified by preparative reverse phase HPLC (Waters Xbridge Prep OBD C18 column, 30-60% MeCN:10 mM NH₄HCO₃ in H₂O) to give rac-trans-1-[2-(3-chlorophenyl)ethyl]-4-[(4-methanesulfonylphenoxy)methyl]-3-methylpyrrolidin-3-ol (Compound 208). ¹H NMR (400 MHz, DMSO-d₆): δ 7.85-7.82 (m, 2H), 7.31-7.11 (m, 6H), 4.58 (s, 1H), 4.29-4.25 (m, 1H), 3.99 (t, J=8.8 Hz, 1H), 3.15 (s, 3H), 2.91 (t, J=8.0 Hz, 1H), 2.74-2.54 (m, 7H), 2.27-2.20 (m, 1H), 1.32 (s, 3H). MS=424.3 [M+H]⁺.

### Step 10: (3R,4S or 3S,4R)-1-[2-(3-chlorophenyl)ethyl]-4-[(4-methanesulfonylphenoxy)methyl]-3-methylpyrrolidin-3-ol (Compound 209) and (3S,4R or 3R,4S)-1-[2-(3-chlorophenyl)ethyl]-4-[(4-methanesulfonylphenoxy)methyl]-3-methylpyrrolidin-3-ol (Compound 210)

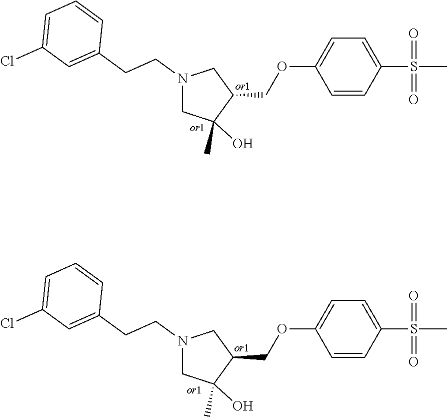 [SMILES: C[C@@]1(O)CN(CCc2cccc(Cl)c2)C[C@@H]1COc1ccc(S(C)(=O)=O)cc1 ; C[C@]1(O)CN(CCc2cccc(Cl)c2)C[C@H]1COc1ccc(S(C)(=O)=O)cc1]

The material obtained from Step 9 (40 mg, 94 μmol) was further purified by chiral SFC (CHIRALPAK IG, 60% isopropanol with 0.1% NH₄OH in CO₂). The first eluting isomer of the title compound (Compound 209): ¹H NMR (400 MHz, DMSO-d₆): δ 7.84 (d, J=8.8 Hz, 2H), 7.32-7.26 (m, 4H), 7.21-7.12 (m, 2H), 4.64 (br s, 1H), 4.30-4.26 (m, 1H), 4.00 (t, J=8.8 Hz, 1H), 3.15 (s, 3H), 3.10-2.90 (m, 2H), 2.85-2.65 (m, 5H), 2.30-2.15 (m, 2H), 1.33 (s, 3H). MS=424.2 [M+H]⁺. The second eluting isomer of the title compound (Compound 210): ¹H NMR (400 MHz, DMSO-d₆): δ 7.84 (d, J=8.8 Hz, 2H), 7.32-7.25 (m, 4H), 7.21-7.12 (m, 2H), 4.65 (br s, 1H), 4.30-4.26 (m, 1H), 4.00 (t, J=9.2 Hz, 1H), 3.15 (s, 3H), 3.05-2.89 (m, 1H), 2.90-2.55 (m, 6H), 2.30-2.15 (m, 2H), 1.33 (s, 3H). MS=424.2 [M+H]⁺.

### Example 29

### 1-[2-(3-chlorophenyl)ethyl]-5-[(4-methanesulfonylphenoxy)methyl]-2-methylpiperazine (Compound 211)

### rac-cis or trans-(2R,5S)-1-[2-(3-chlorophenyl)ethyl]-5-[(4-methanesulfonylphenoxy)methyl]-2-methylpiperazine (Compound 212)

### rac-trans or cis-(2R,5S)-1-[2-(3-chlorophenyl)ethyl]-5-[(4-methanesulfonylphenoxy)methyl]-2-methylpiperazine (Compound 213)

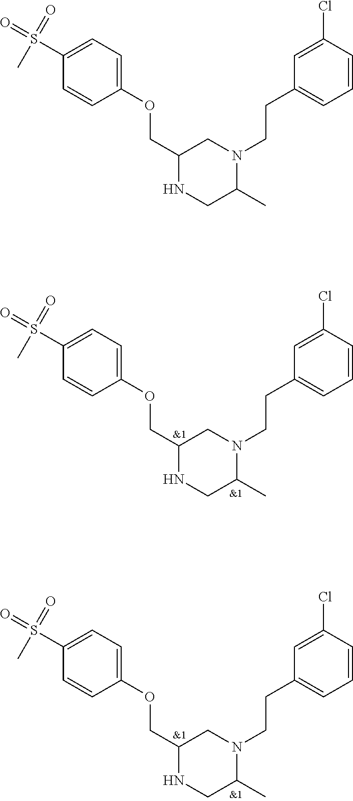 [SMILES: CC1CNC(COc2ccc(S(C)(=O)=O)cc2)CN1CCc1cccc(Cl)c1 ; CC1CNC(COc2ccc(S(C)(=O)=O)cc2)CN1CCc1cccc(Cl)c1 ; CC1CNC(COc2ccc(S(C)(=O)=O)cc2)CN1CCc1cccc(Cl)c1]

### Step 1: 1-(tert-butoxycarbonyl)-4-(3-chlorophenethyl)-5-methylpiperazine-2-carboxylic acid

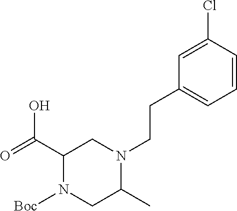 [SMILES: CC1CN(C(=O)OC(C)(C)C)C(C(=O)O)CN1CCc1cccc(Cl)c1]

To a solution of 1-(tert-butoxycarbonyl)-5-methylpiperazine-2-carboxylic acid (950 mg, 3.89 mmol,) and 2-(3-chlorophenyl)acetaldehyde (721 mg, 4.67 mmol) in MeOH (10 mL) and AcOH (0.5 mL) was added 2-methylpyridine borane complex (624 mg, 5.83 mmol). The mixture was stirred at 40° C. for 2 h. The reaction mixture was concentrated under reduced pressure to remove MeOH. The residue was diluted with saturated aqueous NaHCO₃ (30 mL) and extracted with 3:1 DCM/iPrOH (10 mL×3). The combined organic layers were washed with brine (20 mL), dried over Na₂SO₄, filtered and concentrated under reduced pressure. The residue was purified by column chromatography (Biotage 12 g cartridge, 0-9% MeOH/DCM) to give 1-(tert-butoxycarbonyl)-4-(3-chlorophenethyl)-5-methylpiperazine-2-carboxylic acid. MS=383.1 [M+H]⁺.

### Step 2: tert-butyl 4-(3-chlorophenethyl)-2-(hydroxymethyl)-5-methylpiperazine-1-carboxylate

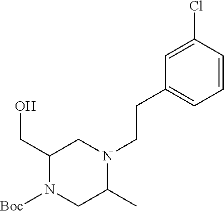 [SMILES: CC1CN(C(=O)OC(C)(C)C)C(CO)CN1CCc1cccc(Cl)c1]

To a solution of 1-(tert-butoxycarbonyl)-4-(3-chlorophenethyl)-5-methylpiperazine-2-carboxylic acid (700 mg, 1.83 mmol) in THF (20 mL) was added BH₃-Me₂S (10 M in DMS, 731 uL, 7.3 mmol). The mixture was stirred at room temperature for 2 h. The reaction was poured into MeOH (20 mL) at 0° C. and stirred for 15 min. The mixture was concentrated under reduced pressure. The residue was diluted with saturated aqueous NaHCO₃ (30 mL) and extracted with 3:1 DCM/iPrOH (10 mL×3). The combined organic layers were washed with brine (20 mL), dried over Na₂SO₄, filtered and concentrated under reduced pressure. The residue was purified by column chromatography (Biotage 12 g cartridge, 2-9% MeOH/DCM) to give tert-butyl 4-(3-chlorophenethyl)-2-(hydroxymethyl)-5-methylpiperazine-1-carboxylate. MS=369.2 [M+H]⁺.

### Step 3: tert-butyl 4-(3-chlorophenethyl)-5-methyl-2-((4-(methylsulfonyl)phenoxy)methyl)piperazine-1-carboxylate

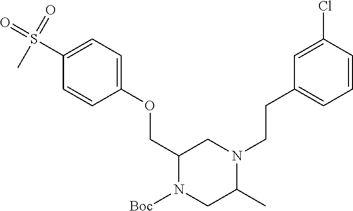 [SMILES: CC1CN(C(=O)OC(C)(C)C)C(COc2ccc(S(C)(=O)=O)cc2)CN1CCc1cccc(Cl)c1]

To a solution of tert-butyl 4-(3-chlorophenethyl)-2-(hydroxymethyl)-5-methylpiperazine-1-carboxylate (550 mg, 1.49 mmol) and 1-fluoro-4-methylsulfonyl-benzene (312 mg, 1.79 mmol) in DMF (10 mL) was added Cs₂CO₃ (972 mg, 2.98 mmol). The mixture was stirred at 100° C. for 40 h. The reaction mixture was cooled to 0° C., quenched by addition of saturated aqueous NH₄Cl (50 mL) and extracted with EtOAc (20 mL×3). The combined organic layers were washed with brine (30 mL), dried over Na₂SO₄, filtered and concentrated under reduced pressure. The residue was purified by column chromatography (Biotage 12 g cartridge, 0-30% EtOAc:Hexane) and further purified by preparative reverse phase HPLC (Phenomenex Luna C18 column, 25-55% MeCN: 0.1% TFA in H₂O) to give tert-butyl 4-(3-chlorophenethyl)-5-methyl-2-((4-(methylsulfonyl)phenoxy)methyl)piperazine-1-carboxylate. MS=523.2 [M+H]⁺.

### Step 4: 1-[2-(3-chlorophenyl)ethyl]-5-[(4-methanesulfonylphenoxy)methyl]-2-methylpiperazine (Compound 211)

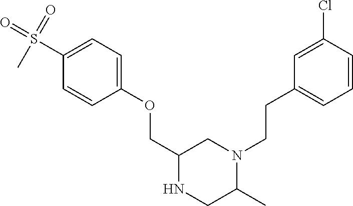 [SMILES: CC1CNC(COc2ccc(S(C)(=O)=O)cc2)CN1CCc1cccc(Cl)c1]

A solution of tert-butyl 4-(3-chlorophenethyl)-5-methyl-2-((4-(methylsulfonyl)phenoxy)methyl)piperazine-1-carboxylate (70 mg, 134 μmol) in HCl/MeOH (5 mL) was stirred for 2 h at room temperature. The mixture was concentrated under reduced pressure to give 1-[2-(3-chlorophenyl)ethyl]-5-[(4-methanesulfonylphenoxy)methyl]-2-methylpiperazine (Compound 211). ¹H NMR (400 MHz, DMSO-d₆): δ 7.92 (d, J=8.8 Hz, 2H), 7.44-7.31 (m, 3H), 7.30-7.23 (m, 3H), 4.60-4.40 (m, 2H), 4.20-4.00 (m, 1H), 3.70-3.62 (m, 5H), 3.37-3.23 (m, 4H), 3.23-2.98 (m, 4H), 1.44 (br s, 3H). MS=423.1 [M+H]⁺.

### Step 5: rac-cis or trans-1-[2-(3-chlorophenyl)ethyl]-5-[(4-methanesulfonylphenoxy)methyl]-2-methylpiperazine (Compound 212) and rac-trans or cis-1-[2-(3-chlorophenyl)ethyl]-5-[(4-methanesulfonylphenoxy)methyl]-2-methylpiperazine (Compound 213)

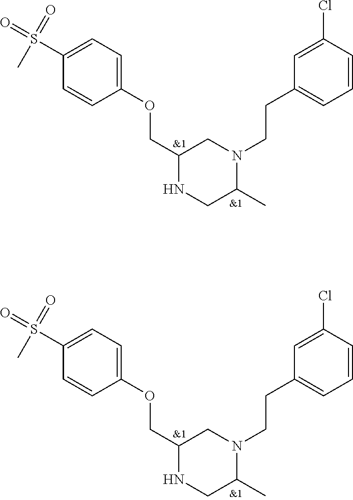 [SMILES: CC1CNC(COc2ccc(S(C)(=O)=O)cc2)CN1CCc1cccc(Cl)c1 ; CC1CNC(COc2ccc(S(C)(=O)=O)cc2)CN1CCc1cccc(Cl)c1]

1-(3-chlorophenethyl)-2-methyl-5-((4-(methylsulfonyl)phenoxy)methyl)piperazine (120 mg, 229 μmol) was further purified by chiral SFC (Chiralpak AD, 46% ethanol with 0.1% NH₄OH in CO₂). The first eluting isomer of the title compound (Compound 212): ¹H NMR (400 MHz, DMSO-d₆): δ 7.87-7.82 (m, 2H), 7.31 (s, 1H), 7.29-7.14 (m, 5H), 4.10-4.01 (m, 2H), 3.16 (s, 3H), 3.10-3.02 (m, 1H), 2.78-2.53 (m, 9H), 2.35-2.17 (m, 1H), 0.95 (d, J=6.4 Hz, 3H). MS=423.1 [M+H]⁺. The second eluting isomer of the title compound (Compound 213): ¹H NMR (400 MHz, DMSO-d₆): δ 7.90 (d, J=8.8 Hz, 2H), 7.35 (s, 1H), 7.29-7.21 (m, 5H), 4.48-4.25 (m, 2H), 3.80-3.65 (m, 1H), 3.18 (s, 3H), 3.10-3.04 (m, 1H), 2.94-2.68 (m, 9H), 1.15-1.03 (m, 3H). MS=423.1 [M+H]⁺.

### Example 30

### (3S,4S)-3-[(4-methanesulfonylphenoxy)methyl]-4-methyl-1-{2-[3-(pentafluoro-λ⁶-sulfanyl)phenyl]ethyl}pyrrolidine (Compound 163)

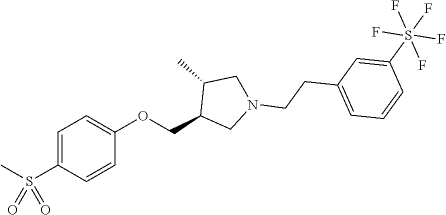 [SMILES: C[C@@H]1CN(CCc2cccc(S(F)(F)(F)(F)F)c2)C[C@H]1COc1ccc(S(C)(=O)=O)cc1]

### Step 1: 1-[(3S,4S)-3-(4-methanesulfonylphenoxymethyl)-4-methylpyrrolidin-1-yl]-2-[3-(pentafluoro-λ⁶-sulfanyl)phenyl]ethenone

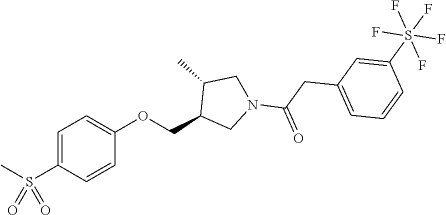 [SMILES: C[C@@H]1CN(C(=O)Cc2cccc(S(F)(F)(F)(F)F)c2)C[C@H]1COc1ccc(S(C)(=O)=O)cc1]

A 20 mL vial was charged with (3S,4S)-3-(4-methanesulfonylphenoxymethyl)-4-methylpyrrolidine hydrochloride (100 mg, 0.327 mmol), [3-(pentafluoro-λ⁶-sulfanyl)phenyl]acetic acid (0.171 g, 0.654 mmol), EDCI HCl (0.094 g, 0.49 mmol), 1-hydroxybenzotriazole hydrate (0.075 g, 0.49 mmol), and DMF (1.6 mL). Lastly added to this mixture was DIEA (0.127 g, 0.981 mmol). The reaction mixture was stirred at room temperature for 3 h. The reaction mixture was poured into H₂O (20 mL) and extracted with EtOAc (3×30 mL). The combined organic layers were washed with brine, dried over MgSO₄, filtered and concentrated under reduced pressure to give crude 1-[(3S,4S)-3-(4-methanesulfonylphenoxymethyl)-4-methylpyrrolidin-1-yl]-2-[3-(pentafluoro-λ⁶-sulfanyl)phenyl]ethanone as a yellow oil. MS=514.0 [M+H]⁺.

### Step 2: (3S,4S)-3-(4-methanesulfonylphenoxymethyl)-4-methyl-1-{2-[3-(pentafluoro-)⁶-sulfanyl)phenyl]ethyl}pyrrolidine (Compound 163)

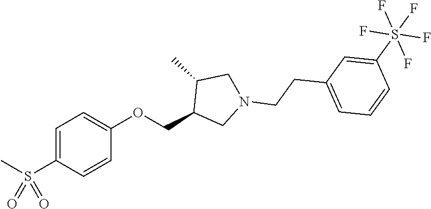 [SMILES: C[C@@H]1CN(CCc2cccc(S(F)(F)(F)(F)F)c2)C[C@H]1COc1ccc(S(C)(=O)=O)cc1]

To a solution of 1-[(3S,4S)-3-(4-methanesulfonylphenoxymethyl)-4-methylpyrrolidin-1-yl]-2-[3-(pentafluoro-λ⁶-sulfanyl)phenyl]ethanone (155 mg, 0.302 mmol) in THF (1.5 mL) at 0° C. was added LiAlH₄ (1 M in THF, 0.905 mL, 0.905 mmol) dropwise. The mixture was stirred at room temperature for 1 h. The reaction was cooled to 0° C., quenched with H₂O and extracted with DCM (20 mL×2). The combined organics were dried over Na₂SO₄, filtered and concentrated. The resulting crude oil was purified by prep-HPLC (5-90% MeCN in H₂O with 0.1% formic acid modifier) to give (3S,4S)-3-(4-methanesulfonylphenoxymethyl)-4-methyl-1-{2-[3-(pentafluoro-λ⁶-sulfanyl)phenyl]ethyl}pyrrolidine (Compound 163). 1H NMR (500 MHz, CDCl₃) δ 7.82-7.78 (m, 2H), 7.57-7.54 (m, 1H), 7.54-7.52 (m, 1H), 7.36-7.30 (m, 2H), 6.97-6.92 (m, 2H), 4.03-3.94 (m, 2H), 3.26 (dd, J=9.9 Hz, 7.4 Hz, 1H), 3.08-2.99 (m, 2H), 2.97-2.86 (m, 7H), 2.45 (t, J=9.4 Hz, 1H), 2.34-2.25 (m, 1H), 2.23-2.14 (m, 1H), 1.12 (d, J=6.7 Hz, 3H). MS=500.1 [M+H]⁺.

### Biological Examples

### Example B-1

This example shows that compounds of the present disclosure are able to inhibit calcium transport by APOL1.

A HEK293 clonal cell line was generated to stably express GCaMP6f, a genetically encoded calcium indicator, and inducibly express APOL1 G2 (HEK T-REx/GCaMP6f/APOL1 G2 K6.3). Cells were maintained in the following standard complete medium: DMEM with 4.5 g/L glucose and sodium pyruvate (BioWhittaker, Lonza, BE12-614F), supplemented with 10% FBS Performance Plus (Gibco, 16000044), 1% penicillin-streptomycin (BioWhittaker, DE17-602E), 2 mM ultraglutamine-1 (BioWhittaker cat. BE 17-605/U1), 50 μg/mL Zeocin (InvivoGen, ant-zn), 2.5 μg/mL Blasticidin (InvivoGen, ant-bl-5), and 25 μg/mL Hygromycin (InvivoGen, ant-hg). Standard propagation conditions consisted of plating 9×10⁶, 4×10⁶, 2×10⁶ cells in a T225 flasks to be processed after 2, 3, or 4 days, respectively.

A source plate was generated containing 20 serially diluted compounds in DMSO (duplicate 8-point dose response). Next, 0.8 μL of compounds were transferred from the source plate to a destination plate prefilled with 79.2 μL of Ca²⁺ free Tyrode's buffer (130 mM NaCl, 5 mM KCl, 1 mM MgCl₂, 5 mM NaHCO₃, 20 mM HEPES at pH 7.4). The destination plate was placed on a plate shaker (5 seconds at 2000 rpm) to mix. This process resulted in a destination plate with 2× concentrated compound solutions. All transfer and mixing steps were conducted with an CyBi®-Well dispenser.

Cells were split by gently washing with DPBS (Euroclone, ECB4004L), followed by a 5-minute incubation (humidified, 37° C. with 5% CO₂) with trypsin-EDTA solution (Euroclone, ECB3052D). Detached cells were diluted with standard complete medium without selective agents, counted, and plated in a 384 MTP microplate (GR4332CPL, Twin Helix) (10,000 cells/well in 25 μl/well) using a MATRIX WellMate dispenser. Plates were placed into a humidified incubator (37° C. with 5% CO₂) overnight. The following day, 20 μL of doxycycline (Sigma, D9891) at 20 ng/mL in standard complete medium was added to cells with a CyBi @Drop dispenser to induce APOL1 G2 expression. After a 6-hour incubation (humidified, 37° C. with 5% CO₂), cells were washed 3 times with Ca²⁺ free Tyrode's Buffer (130 mM NaCl, 5 mM KCl, 1 mM MgCl₂, 5 mM NaHCO₃, 20 mM HEPES at pH 7.4) using a BIOTEK Microplate washer, such that 10 μL of buffer remained in each well after the final wash. Assay plates were then stored at room temperature for 10 minutes. Next, 10 μL of diluted compounds were transferred to the assay plate from the 2× compound plate using a CyBi®-Well dispenser. Compound incubation was then carried out at room temperature for 10 minutes. The assay plate was transferred to the FLIProom temperature^{ETRA} and 20 μL of 10 mM Ca²⁺ (final concentration=5 mM) Tyrode's buffer was injected.

Table B1 below summarizes the data from this experiment. Unless otherwise specified, AC₅₀ and values are reported as the geometric mean of at least 2 assay runs on separate days. Each run represents the average of a technical replicate, where each compound was assayed twice in the same plate. A superscript † symbol indicates a value from the average of a technical replicate from a single assay run, where each compound was assayed twice in the same plate.

The AC₅₀ values in Table B1 below reflect the compound's ability to prevent calcium influx by inhibiting APOL1. As shown in the table, compounds of the present disclosure are able to potently inhibit APOL1-mediated calcium transport at sub micromolar concentrations. Compounds in Table B1 are referred to by the corresponding Compound Number in Table 1, which is also referred to in the synthetic examples. When one or more of the numbered compounds are identified by stereochemistry (for example, (R)- or (S)—), the specific stereoisomer for which data is provided in Table B1 may be identified by the elution order of such compound as described in the synthetic examples. To illustrate, Compound 2 is the first-eluting enantiomer of step 4 of Example 1 and Compound 3 is the second-eluting enantiomer of step 4 of Example 1. Further, by way of illustration, Compound 27 is the first-eluting enantiomeric mixture in step 4 of Example 7 and Compound 28 is the second-eluting enantiomeric mixture in step 4 of Example 7. Then, Compound 27 is separated into Compound 29 (the first-eluting enantiomer) and Compound 30 (the second-eluting enantiomer) in Example 8, and Compound 28 is separated into Compound 31 (the first-eluting enantiomer) and Compound 32 (the second-eluting enantiomer) in Example 8. Absolute stereochemistry of such compounds may be identified by methods known in the art.

TABLE B1

| Compound | APOL1 G2 FLIPR |
|---|---|
| No. | AC₅₀ (μM) |
| 1 | ≤0.253 |
| 2 | 1.01^{†} |
| 3 | 0.205^{†} |
| 4 | 1.07^{†} |
| 5 | 10.1^{†} |
| 6 | 0.942 |
| 7 | <0.253 |
| 8 | 0.266 |
| 9 | 0.151^{†} |
| 10 | 1.72^{†} |
| 11 | 1.19 |
| 12 | 2.39 |
| 13 | 0.143 |
| 14 | 3.34^{†} |
| 15 | 0.287^{†} |
| 16 | 0.231^{†} |
| 17 | 1.37^{†} |
| 18 | 5.73^{†} |
| 19 | 0.48 |
| 20 | 1.76^{†} |
| 21 | 0.623 |
| 22 | 6.41^{†} |
| 23 | 0.64^{†} |
| 24 | 0.501 |
| 25 | 0.469^{†} |
| 26 | 1.54^{†} |
| 27 | 1.50^{†} |
| 28 | 2.00^{†} |
| 29 | 1.98^{†} |
| 30 | 0.864^{†} |
| 31 | 1.02^{†} |
| 32 | 0.731^{†} |
| 33 | 10.8^{†} |
| 34 | 0.991^{†} |
| 35 | 3.78^{†} |
| 36 | 14.2^{†} |
| 37 | 3.84^{†} |
| 38 | <0.253 |
| 39 | 0.249 |
| 40 | <0.118^{†} |
| 41 | 2.11^{†} |
| 42 | 0.477 |
| 43 | 0.237^{†} |
| 44 | 2.22^{†} |
| 45 | 0.0712 |
| 46 | 0.0902 |
| 47 | 0.434 |
| 48 | 1.92^{†} |
| 49 | 0.847 |
| 50 | 1.32^{†} |
| 51 | 0.727 |
| 52 | 0.079^{†} |
| 53 | 0.856^{†} |
| 54 | <0.114 |
| 55 | 1.29^{†} |
| 56 | 0.580^{†} |
| 57 | <5.00 |
| 58 | 0.0727^{†} |
| 59 | 0.245 |
| 60 | <0.267 |
| 61 | 0.376 |
| 62 | 0.396 |
| 63 | 0.637 |
| 64 | 0.570 |
| 65 | 0.258^{†} |
| 66 | 0.468 |
| 67 | 0.34^{†} |
| 68 | 0.916 |
| 69 | 12.87^{†} |
| 70 | 0.295^{†} |
| 71 | 0.943 |
| 72 | 0.314^{†} |
| 73 | 3.06^{†} |
| 74 | 1.29^{†} |
| 75 | 0.558 |
| 76 | 2.83 |
| 77 | 1.67^{†} |
| 78 | 7.01^{†} |
| 79 | 1.29^{†} |
| 80 | 13.9^{†} |
| 81 | >25.0 |
| 82 | 4.35 |
| 83 | >25.0^{†} |
| 84 | >25.0^{†} |
| 85 | 8.48^{†} |
| 86 | 0.557 |
| 87 | 0.352 |
| 88 | 0.338 |
| 89 | 0.449 |
| 90 | 0.515 |
| 91 | 0.472 |
| 92 | 0.682^{†} |
| 93 | 6.54 |
| 94 | 1.62 |
| 95 | 1.54 |
| 96 | 3.77 |
| 97 | 0.314^{†} |
| 98 | 3.92 |
| 99 | 4.14^{†} |
| 100 | 1.26 |
| 101 | 1.30^{†} |
| 102 | 2.19^{†} |
| 103 | 10.9^{†} |
| 104 | <0.382 |
| 105 | 0.946^{†} |
| 106 | 2.79^{†} |
| 107 | 0.0919 |
| 108 | 0.144 |
| 109 | <0.105 |
| 110 | 0.0914 |
| 111 | 0.494 |
| 112 | >0.159 |
| 113 | 0.907 |
| 114 | 0.709 |
| 115 | 0.756 |
| 116 | 0.374 |
| 117 | 1.08 |
| 118 | 0.936 |
| 119 | 0.784 |
| 120 | 0.461 |
| 121 | 0.270 |
| 122 | 0.709 |
| 123 | 0.376 |
| 124 | 0.148 |
| 125 | 2.35 |
| 126 | 0.704 |
| 127 | 2.48 |
| 128 | 0.349 |
| 129 | 1.04 |
| 130 | 2.17 |
| 131 | 0.394 |
| 132 | 0.510 |
| 133 | 0.0383 |
| 134 | 0.277 |
| 135 | >12.5 |
| 136 | 0.207 |
| 137 | >10.0 |
| 138 | 2.50 |
| 139 | >10.0 |
| 140 | 0.107 |
| 141 | 0.737 |
| 142 | 1.23 |
| 143 | 3.16 |
| 144 | 0.423 |
| 145 | 0.356 |
| 146 | 0.937 |
| 147 | 1.11 |
| 148 | 0.615 |
| 149 | 0.0998 |
| 150 | 0.721 |
| 151 | 0.31 |
| 152 | 1.99 |
| 153 | 0.374 |
| 154 | 0.098 |
| 155 | 1.98 |
| 156 | 0.311 |
| 157 | 0.441 |
| 158 | 0.352 |
| 159 | 0.358 |
| 160 | 0.382 |
| 161 | 0.878 |
| 162 | 0.233 |
| 163 | 1.15 |
| 164 | 0.364 |
| 165 | 0.409 |
| 166 | 0.256 |
| 167 | 0.314 |
| 168 | 0.179 |
| 169 | 0.0797 |
| 170 | 0.0375 |
| 171 | 0.212 |
| 172 | 0.104 |
| 173 | 0.491 |
| 174 | 0.157 |
| 175 | <0.0728 |
| 176 | 0.063 |
| 177 | 2.86 |
| 181 | <0.0961 |
| 182 | 0.396 |
| 183 | 0.149 |
| 184 | 0.215 |
| 185 | 0.322 |
| 186 | 6.98 |
| 187 | 0.198 |
| 188 | 0.511 |
| 189 | 0.634 |
| 190 | 0.170 |
| 191 | 0.228 |
| 192 | 0.260 |
| 193 | 1.45 |
| 194 | 0.151 |
| 195 | 0.236 |
| 196 | 0.108 |
| 197 | 0.334 |
| 198 | 0.197 |
| 199 | 0.817 |
| 200 | 0.836 |
| 201 | 0.620 |
| 202 | 0.860 |
| 203 | 9.30 |
| 204 | 0.456 |
| 205 | <0.236 |
| 206 | >25.0 |
| 207 | 0.340 |
| 208 | <0.420 |
| 209 | 3.74 |
| 210 | 0.898 |
| 211 | <0.118 |
| 212 | 2.55 |
| 213 | 0.877 |
| 214 | 0.428 |
| 215 | 0.229 |

### Example B-2

This example shows that the compounds of the present disclosure are able to reduce cell death caused by overexpression of APOL1.

A HEK293 clonal cell line overexpressing APOL1 G2 (HEK293/T-REx APOL1 G2/clone #2) was maintained in 1×DMEM-GlutaMax (Gibco, 10569-010) media with 10% tetracycline-free FBS (Takara Bio USA, 631101), 5 μg/mL Blasticidin (Gibco, A1113903), and 100 μg/mL Zeocin (Invitrogen, R25001) in T75 flasks. In preparation for the assay, this media was aspirated and 2 mL of prewarmed TrypLE Express (Gibco, 12605-010) was added to a flask to detach cells. The flask was then incubated (humidified, 37° C. with 5% CO2) for 3-5 minutes. Afterwards, 8 mL of prewarmed cell assay media (lx DMEM-GlutaMax media with 10% tetracycline-free FBS) was added to the trypsinized cells. The suspension was gently mixed, and cells were counted using a Countess Cell Counting Chamber (Invitrogen). The suspension was diluted using cell assay media to generate a working stock solution (166,667 cells/mL). Using a MultiDrop Combi (Thermo Electron Corp), 30 μL (final cell density=5,000 cells/well) of the working stock solution was dispensed into each well of white 384-well assay ready plates (Nunc™, 164610) containing 6 ng/mL doxycycline, to induce APOL1 expression, and compound. All compounds were plated in a duplicate 8-point dilution series that consisted of 3-fold stepwise dilutions (0.5% DMSO final). Assay plates were incubated (humidified, 37° C. with 5% CO2) for 17 hours. After the incubation, the plates were equilibrated at room temperature for 1 hour. Next, 15 μl of CellTiter-Glo® reagent (Promega, G7570) was added to each well using a MultiDrop Combi. Plates were placed on an orbital shaker (500 rpm) for 5 minutes to induce cell lysis and then incubated at room temperature for 10 minutes. Luminescence was measured on an Envision plate reader. Collaborative Drug Discovery software was utilized for graphing data. Plots were generated using a four parameter logistic curve fit.

Table B2 below provides the results from this experiment. Unless otherwise specified, EC₅₀ values are reported as the geometric mean of at least 2 assay runs on separate days. Each run represents the average of a technical replicate, where each compound was assayed twice in the same plate. A superscript t symbol indicates a value from the average of a technical replicate from a single assay run, where each compound was assayed twice in the same plate. Compounds in Table B2 are referred to by the corresponding Compound Number in Table 1, which is also referred to in the synthetic examples. When one or more of the numbered compounds are identified by stereochemistry (for example, (R)- or (S)—), the specific stereoisomer for which data is provided in Table B2 may be identified by the elution order of such compound as described in the synthetic examples. To illustrate, Compound 2 is the first-eluting enantiomer of step 4 of Example 1 and Compound 3 is the second-eluting enantiomer of step 4 of Example 1. Further, by way of illustration, Compound 27 is the first-eluting enantiomeric mixture in step 4 of Example 7 and Compound 28 is the second-eluting enantiomeric mixture in step 4 of Example 7. Then, Compound 27 is separated into Compound 29 (the first-eluting enantiomer) and Compound 30 (the second-eluting enantiomer) in Example 8, and Compound 28 is separated into Compound 31 (the first-eluting enantiomer) and Compound 32 (the second-eluting enantiomer) in Example 8. Absolute stereochemistry of such compounds may be identified by methods known in the art.

Rescue EC₅₀ values reported in Table B2 below represent the half-maximal effective concentration for reversal of cell death caused by overexpression of APOL1. This example demonstrates that compounds of the present disclosure are able to reduce cell death caused by overexpression of APOL1 at sub micromolar concentration.

TABLE B2

| Compound | APOL1 G2 HEK293 |
|---|---|
| No. | Rescue EC₅₀ (μM) |
| 1 | 0.528 |
| 2 | 2.96 |
| 3 | 0.295 |
| 4 | 2.35 |
| 5 | 5.37 |
| 6 | 0.495 |
| 7 | 0.322 |
| 8 | 0.132 |
| 9 | 0.263 |
| 10 | 2.45 |
| 11 | 1.00 |
| 12 | 3.00 |
| 13 | 0.445 |
| 14 | >30.0 |
| 15 | 0.567^{†} |
| 16 | 0.329^{†} |
| 17 | 0.671 |
| 18 | 6.32 |
| 19 | 0.374 |
| 20 | 1.36 |
| 21 | 1.02 |
| 22 | 6.59 |
| 23 | 0.677 |
| 24 | 0.334 |
| 25 | 1.01 |
| 26 | 2.24 |
| 27 | 2.13 |
| 28 | 2.34 |
| 29 | >12.2 |
| 30 | 2.41 |
| 31 | >7.89 |
| 32 | 5.77 |
| 33 | >8.67 |
| 34 | 0.779 |
| 35 | 6.13 |
| 36 | >30 |
| 37 | >6.15 |
| 38 | 0.407 |
| 39 | 0.904 |
| 40 | 0.746 |
| 41 | 2.82 |
| 42 | 0.059 |
| 43 | 0.031 |
| 44 | 2.43 |
| 45 | <0.0137 |
| 46 | 0.021 |
| 47 | 0.516 |
| 48 | 2.54 |
| 49 | 0.957 |
| 50 | 2.02 |
| 51 | 1.49 |
| 52 | 0.124 |
| 53 | 1.12 |
| 54 | >30 |
| 55 | 1.68 |
| 56 | 1.68 |
| 57 | 1.68 |
| 58 | <0.041 |
| 59 | 1.06 |
| 60 | 0.306 |
| 61 | 0.933 |
| 62 | 0.642 |
| 63 | 3.96^{†} |
| 64 | 1.12^{†} |
| 65 | 0.955 |
| 66 | 1.38^{†} |
| 67 | 1.35 |
| 68 | 0.591 |
| 69 | >30 |
| 70 | 0.303 |
| 71 | 1.96 |
| 72 | 1.22 |
| 73 | >17.8 |
| 74 | 2.58 |
| 75 | 1.46 |
| 76 | >6.28 |
| 77 | >6.30 |
| 78 | >30.0 |
| 79 | >13.7 |
| 80 | 13.0 |
| 81 | >30.0 |
| 82 | >24.3 |
| 83 | >30.0 |
| 84 | >30.0 |
| 85 | >18.0 |
| 86 | >4.11 |
| 87 | 1.85 |
| 88 | 0.698 |
| 89 | 2.02 |
| 90 | 1.46 |
| 91 | 1.04 |
| 92 | >9.79 |
| 93 | >30 |
| 94 | 8.37 |
| 95 | 6.90 |
| 96 | >30.0 |
| 97 | 0.46 |
| 98 | >20 |
| 99 | >30.0 |
| 100 | 2.73 |
| 101 | 10.1 |
| 102 | >21.8 |
| 103 | >30.0 |
| 104 | 1.86 |
| 105 | >12.4 |
| 106 | 6.24 |
| 107 | 0.0809 |
| 108 | 0.0504 |
| 109 | 0.500 |
| 110 | 0.135 |
| 111 | 1.88 |
| 112 | 0.0298 |
| 113 | 1.07 |
| 114 | 0.439 |
| 115 | 0.0447 |
| 116 | 0.076 |
| 117 | 1.24 |
| 118 | 4.04 |
| 119 | 0.133 |
| 120 | 0.195 |
| 121 | 0.100 |
| 122 | 1.34 |
| 123 | 0.300 |
| 124 | 0.242 |
| 125 | 1.55 |
| 126 | 0.560 |
| 127 | >10.0 |
| 128 | 0.366 |
| 129 | 0.723 |
| 130 | >10.0 |
| 131 | 0.460 |
| 132 | 0.0437 |
| 133 | 0.135 |
| 134 | 0.908 |
| 135 | 5.73 |
| 136 | 0.11 |
| 137 | 4.62 |
| 138 | 1.97 |
| 139 | 3.23 |
| 140 | 0.0459 |
| 141 | 0.0789 |
| 142 | 0.131 |
| 143 | 0.349 |
| 144 | 0.256 |
| 145 | 0.243 |
| 146 | 0.273 |
| 147 | 0.618 |
| 148 | 0.0808 |
| 149 | 0.272 |
| 150 | 1.47 |
| 151 | 1.34 |
| 152 | 4.72 |
| 153 | 0.0278 |
| 154 | 0.167 |
| 155 | 2.49 |
| 156 | 0.178 |
| 157 | 0.0389 |
| 158 | 0.039 |
| 159 | 0.0249 |
| 160 | 0.584 |
| 161 | 0.191 |
| 162 | 0.0279 |
| 163 | >0.385 |
| 164 | 0.0341 |
| 165 | 0.0302 |
| 166 | 0.0179 |
| 167 | 0.0672 |
| 168 | 0.126 |
| 169 | 0.182 |
| 170 | 0.076 |
| 171 | 0.146 |
| 172 | 0.203 |
| 173 | 0.201 |
| 174 | 0.0361 |
| 175 | 0.143 |
| 176 | <0.0472 |
| 177 | 1.83 |
| 181 | 0.325 |
| 182 | 0.399 |
| 183 | 0.157 |
| 184 | 0.622 |
| 185 | 0.239 |
| 186 | 0.631 |
| 187 | 0.436 |
| 188 | 0.303 |
| 189 | 0.922 |
| 190 | 0.291 |
| 191 | 0.663 |
| 192 | 0.915 |
| 193 | 0.782 |
| 194 | 0.107 |
| 195 | 0.226 |
| 196 | 0.110 |
| 197 | 0.369 |
| 198 | 0.0715 |
| 199 | 2.44 |
| 200 | 1.99 |
| 201 | 1.68 |
| 202 | 0.089 |
| 203 | 3.54 |
| 204 | 0.0423 |
| 205 | 1.59 |
| 206 | >30.0 |
| 207 | 0.933 |
| 208 | 3.57 |
| 209 | >19.5 |
| 210 | 3.12 |
| 211 | 1.36 |
| 212 | >14.3 |
| 213 | 0.964 |
| 214 | 0.679 |
| 215 | 0.0422 |

All publications, patent applications, patents, and other references mentioned herein are expressly incorporated by reference in their entireties, to the same extent as if each were incorporated by reference individually.

It is to be understood that, while the disclosure has been described in conjunction with the above embodiments, the foregoing description and examples are intended to illustrate and not limit the scope of the disclosure. Other aspects, advantages and modifications within the scope of the disclosure will be apparent to those skilled in the art to which the disclosure pertains.

What is claimed is:

1. A compound of formula (A′): 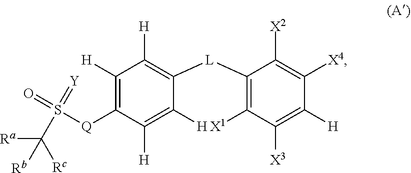
or a stereoisomer or a tautomer thereof, or a pharmaceutically acceptable salt of any of the foregoing, wherein:
Q is absent or is —N—(C₁₋₆alkyl);
Y is O or —N—(C₁₋₆alkyl),
provided that, when Q is —N(C₁₋₆alkyl), then Y is O;
R^{a}, R^{b}, and R^{c} are each independently H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl,
or any two of R^{a}, R^{b}, and R^{c} are taken, together with the atoms to which they are attached, to form a C₃₋₆cycloalkyl or a 3-6 membered heterocyclyl, and the other of R^{a}, R^{b}, and R^{c} is H or C₁₋₆alkyl, wherein the C₁₋₆alkyl of R^{a}, R^{b}, or R^{c} is independently optionally substituted with one or more —OH, C₁₋₆alkoxy, or —S(O)₂-C₁₋₆alkyl;
L is selected from the group consisting of: 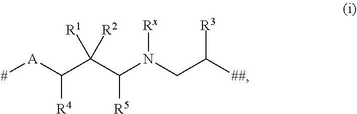
wherein
A is O, NH, N(C₁₋₆alkyl), CH₂, or CH(C₁₋₆alkyl);
R^{x} is H,
or R^{x} is taken together with one of R¹ and R², and the atoms to which they are attached, to form a 3-8 membered heterocyclyl, wherein the 3-8 membered heterocyclyl is substituted with n independently selected R^{g} substituents, wherein n is an integer from 0-6, and R^{g} is —OH, halo, C₁₋₆alkyl, or C₁₋₆alkoxy;
R¹ and R² are independently H, halo, or —OH,
or one of R¹ and R² is taken together with R^{x}, and the atoms to which they are attached, to form a 3-8 membered heterocyclyl, wherein the 3-8 membered heterocyclyl is substituted with n independently selected R^{g} substituents, wherein n is an integer from 0-6, and R^{g} is —OH, halo, C₁₋₆alkyl, or C₁₋₆alkoxy, and the other of R¹ and R² is H, halo, or —OH;
R³ is H, —OH, halo, or C₁₋₆alkoxy; and
R⁴ and R⁵ are independently H,
or R⁴ and R⁵ are taken, together with the atoms to which they are attached, to form a C₃₋₈cycloalkyl,
provided that either:
(1) R^{x} is taken together with one of R¹ and R², and the atoms to which they are attached, to form a 3-8 membered heterocyclyl, wherein the 3-8 membered heterocyclyl is substituted with n independently selected R^{g} substituents, wherein n is an integer from 0-6, and R^{g} is —OH, halo, C₁₋₆alkyl, or C₁₋₆alkoxy, or
(2) R⁴ and R⁵ are taken, together with the atoms to which they are attached, to form a C₃₋₈cycloalkyl, 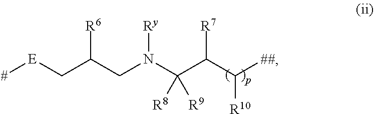
wherein
E is O, NH, N(C₁₋₆alkyl), CH₂, or CH(C₁₋₆alkyl);
p is 0 or 1,
provided that, when p is 1, then E is O;
R⁶ is H or —OH;
R^{y} is H,
or R^{y} is taken together with R⁷, and the atoms to which they are attached, to form a 3-8 membered heterocyclyl,
or R^{y} is taken together with one of R⁸ and R⁹, and the atoms to which they are attached, to form a 3-8 membered heterocyclyl;
R⁷ is H,
or R⁷ is taken together with R^{y}, and the atoms to which they are attached, to form a 3-8 membered heterocyclyl;
R⁸ and R⁹ are independently H or C₁₋₆alkyl,
or one of R⁸ and R⁹ is taken together with R^{y}, and the atoms to which they are attached, to form a 3-8 membered heterocyclyl, and the other of R⁸ and R⁹ is H or C₁₋₆alkyl,
or one of R⁸ and R⁹ is taken together with R¹⁰, and the atoms to which they are attached, to form a C₃₋₈cycloalkyl, and the other of R⁸ and R⁹ is H or C₁₋₆alkyl; and
R¹⁰ is H,
or R¹⁰ is taken together with one of R⁸ and R⁹, and the atoms to which they are attached, to form a C₃₋₈cycloalkyl,
provided that:
(1) R^{y} is taken together with R⁷, and the atoms to which they are attached, to form a 3-8 membered heterocyclyl, or
(2) R^{y} is taken together with one of R⁸ and R⁹, and the atoms to which they are attached, to form a 3-8 membered heterocyclyl, or
(3) one of R⁸ and R⁹ is taken together with R¹⁰ and the atoms to which they are attached, to form a C₃₋₈cycloalkyl, and 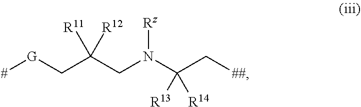
wherein
G is O, NH, N(C₁₋₆alkyl), CH₂, or CH(C₁₋₆alkyl);
R^{z} is H or C₁₋₆alkyl, wherein the C₁₋₆alkyl is optionally substituted with one or more C₃₋₈cycloalkyl;
R¹¹ and R¹² are independently H, —OH, halo, or C₁₋₆alkyl; and
R¹³ and R¹⁴ are independently H, C₁₋₆alkyl, or C₃₋₈cycloalkyl,
or R¹³ and R¹⁴ are taken, together with the atoms to which they are attached, to form a 3-8 membered heterocyclyl,
wherein, for each of (i)-(iii), #denotes the point of attachment to the phenyl ring bearing moiety Q, and ##denotes the point of attachment to the phenyl ring bearing moieties X¹-X⁴; and
X¹, X², X³, and X⁴ are, independently of each other, H, halo, —CN, C₁₋₆alkyl, C₁₋₆alkoxy, or SF₅, wherein the C₁₋₆alkyl or C₁₋₆alkoxy is optionally substituted with one or more halo,
provided that at least one of X¹, X², X³, and X⁴ is halo, —CN, C₁₋₆alkyl, C₁₋₆alkoxy, or SF₅, wherein the C₁₋₆alkyl or C₁₋₆alkoxy is optionally substituted with one or more halo.

2. The compound of claim 1, wherein the compound is a compound of formula (A): 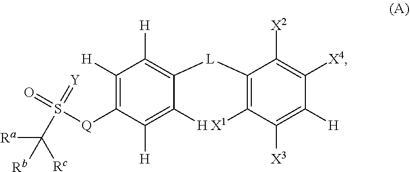
or a stereoisomer or a tautomer thereof, or a pharmaceutically acceptable salt of any of the foregoing, wherein:
X¹, X², X³, and X⁴ are, independently of each other, H, halo, —CN, C₁₋₆alkyl, or C₁₋₆alkoxy, wherein the C₁₋₆alkyl or C₁₋₆alkoxy is optionally substituted with one or more halo,
provided that at least one of X¹, X², X³, and X⁴ is halo, —CN, C₁₋₆alkyl, or C₁₋₆alkoxy, wherein the C₁₋₆alkyl or C₁₋₆alkoxy is optionally substituted with one or more halo.

3. The compound of claim 1 or claim 2, or a stereoisomer or tautomer thereof, or a pharmaceutically acceptable salt of any of the foregoing, wherein L is, 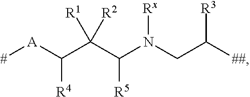
such that the compound is of formula (I): 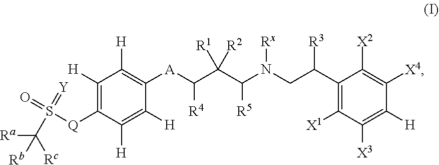
or a stereoisomer or tautomer thereof, or a pharmaceutically acceptable salt of any of the foregoing.

4. The compound of claim 3, or a stereoisomer or tautomer thereof, or a pharmaceutically acceptable salt of any of the foregoing, wherein A is O, such that the compound is of formula (I-A): 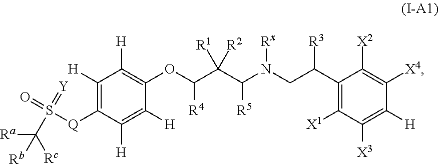
or a stereoisomer or tautomer thereof, or a pharmaceutically acceptable salt of any of the foregoing.

5. The compound of claim 4, or a stereoisomer or tautomer thereof, or a pharmaceutically acceptable salt of any of the foregoing, wherein R^{x} is taken together with one of R¹ and R², and the atoms to which they are attached, to form a 3-8 membered heterocyclyl, wherein the 3-8 membered heterocyclyl is substituted with n independently selected R^{g} substituents, wherein n is an integer from 0-6, and R^{g} is —OH, halo, C₁₋₆alkyl, or C₁₋₆alkoxy.

6. The compound of claim 4, or a stereoisomer or tautomer thereof, or a pharmaceutically acceptable salt of any of the foregoing, wherein R^{x} is taken together with one of R¹ and R², and the atoms to which they are attached, to form a 5-6 membered heterocyclyl, wherein the 5-6 membered heterocyclyl is substituted with n independently selected R^{g} substituents, wherein n is an integer from 0-6, and R^{g} is —OH, halo, C₁₋₆alkyl, or C₁₋₆alkoxy.

7. The compound of claim 5 or claim 6, or a stereoisomer or tautomer thereof, or a pharmaceutically acceptable salt of any of the foregoing, wherein the compound is of formula (I-B1): 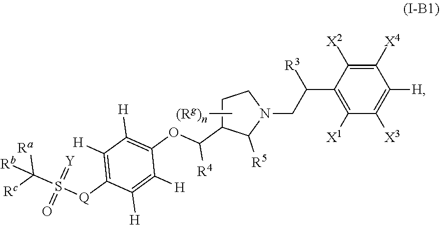
or a stereoisomer or tautomer thereof, or a pharmaceutically acceptable salt of any of the foregoing.

8. The compound of claim 7, or a stereoisomer or tautomer thereof, or a pharmaceutically acceptable salt of any of the foregoing, wherein n is 0.

9. The compound of claim 7, or a stereoisomer or tautomer thereof, or a pharmaceutically acceptable salt of any of the foregoing, wherein n is 1 or 2.

10. The compound of claim 7 or claim 9, or a stereoisomer or tautomer thereof, or a pharmaceutically acceptable salt of any of the foregoing, wherein each R^{g} is, independently at each occurrence, C₁₋₆alkyl.

11. The compound of any one of claims 7, 9, and 10, or a stereoisomer or tautomer thereof, or a pharmaceutically acceptable salt of any of the foregoing, wherein R^{g} is, independently at each occurrence, methyl.

12. The compound of claim 5 or claim 6, or a stereoisomer or tautomer thereof, or a pharmaceutically acceptable salt of any of the foregoing, wherein the compound is of formula (I-C1): 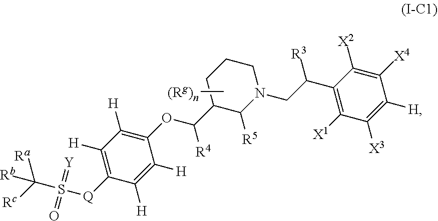
or a stereoisomer or tautomer thereof, or a pharmaceutically acceptable salt of any of the foregoing.

13. The compound of claim 5 or claim 6, or a stereoisomer or tautomer thereof, or a pharmaceutically acceptable salt of any of the foregoing, wherein the compound is of formula (I-D1): 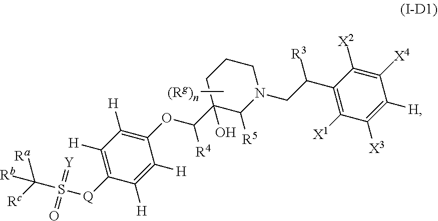
wherein or a stereoisomer or tautomer thereof, or a pharmaceutically acceptable salt of any of the foregoing.

14. The compound of claim 12 or claim 13, or a stereoisomer or tautomer thereof, or a pharmaceutically acceptable salt of any of the foregoing, wherein n is 0.

15. The compound of claim 12 or claim 13, or a stereoisomer or tautomer thereof, or a pharmaceutically acceptable salt of any of the foregoing, wherein n is 1 or 2.

16. The compound of any one of claims 12, 13, and 15, or a stereoisomer or tautomer thereof, or a pharmaceutically acceptable salt of any of the foregoing, wherein R^{g} is, independently at each occurrence, —OH or C₁₋₆alkoxy.

17. The compound of any one of claims 12, 13, 15, and 16, or a stereoisomer or tautomer thereof, or a pharmaceutically acceptable salt of any of the foregoing, wherein R^{g} is, independently at each occurrence, —OH or methoxy.

18. The compound of claim 5 or claim 6, or a stereoisomer or tautomer thereof, or a pharmaceutically acceptable salt of any of the foregoing, wherein the compound is of formula (I-E1): 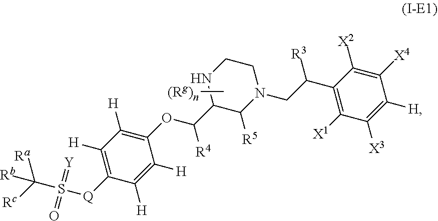
or a stereoisomer or tautomer thereof, or a pharmaceutically acceptable salt of any of the foregoing.

19. The compound of claim 18, or a stereoisomer or tautomer thereof, or a pharmaceutically acceptable salt of any of the foregoing, wherein n is 0.

20. The compound of claim 5, or a stereoisomer or tautomer thereof, or a pharmaceutically acceptable salt of any of the foregoing, wherein the compound is of formula (I-F1): 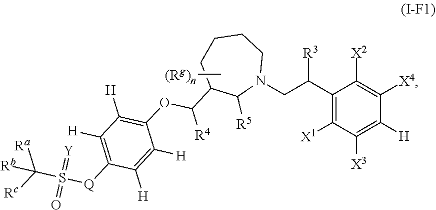
or a stereoisomer or tautomer thereof, or a pharmaceutically acceptable salt of any of the foregoing.

21. The compound of claim 20, or a stereoisomer or tautomer thereof, or a pharmaceutically acceptable salt of any of the foregoing, wherein n is 0.

22. The compound of claim 5, or a stereoisomer or tautomer thereof, or a pharmaceutically acceptable salt of any of the foregoing, wherein the compound is of formula (I-G1): 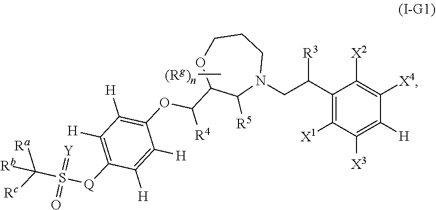
or a stereoisomer or tautomer thereof, or a pharmaceutically acceptable salt of any of the foregoing.

23. The compound of claim 22, or a stereoisomer or tautomer thereof, or a pharmaceutically acceptable salt of any of the foregoing, wherein n is 0.

24. The compound of 4, or a stereoisomer or tautomer thereof, or a pharmaceutically acceptable salt of any of the foregoing, wherein R⁴ and R⁵ are taken, together with the atoms to which they are attached, to form a C₃₋₈cycloalkyl.

25. The compound of claim 4 or claim 24, or a stereoisomer or tautomer thereof, or a pharmaceutically acceptable salt of any of the foregoing, wherein the compound is of formula (I-H1): 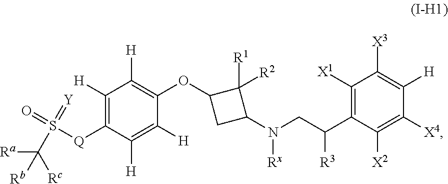
or a stereoisomer or tautomer thereof, or a pharmaceutically acceptable salt of any of the foregoing.

26. The compound of claim 25, or a stereoisomer or tautomer thereof, or a pharmaceutically acceptable salt of any of the foregoing, wherein R¹, R², and R³ are each H.

27. The compound of claim 3, or a stereoisomer or tautomer thereof, or a pharmaceutically acceptable salt of any of the foregoing, wherein A is CH₂, such that the compound is of formula (I-A3): 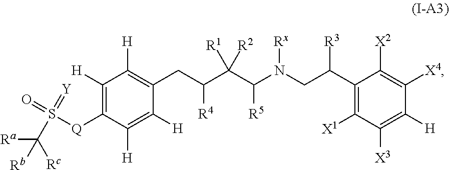
or a stereoisomer or tautomer thereof, or a pharmaceutically acceptable salt of any of the foregoing.

28. The compound of claim 27, or a stereoisomer or tautomer thereof, or a pharmaceutically acceptable salt of any of the foregoing, wherein R^{x} is taken together with one of R¹ and R², and the atoms to which they are attached, to form a 3-8 membered heterocyclyl, wherein the 3-8 membered heterocyclyl is substituted with n independently selected R^{g} substituents, wherein n is an integer from 0-6, and R^{g} is —OH, halo, C₁₋₆alkyl, or C₁₋₆alkoxy, and the other of R¹ and R² is H, halo, or —OH.

29. The compound of claim 27, or a stereoisomer or tautomer thereof, or a pharmaceutically acceptable salt of any of the foregoing, wherein R^{x} is taken together with one of R¹ and R², and the atoms to which they are attached, to form a 5-6 membered heterocyclyl, wherein the 5-6 membered heterocyclyl is substituted with n independently selected R^{g} substituents, wherein n is an integer from 0-6, and R^{g} is —OH, halo, C₁₋₆alkyl, or C₁₋₆alkoxy, and the other of R¹ and R² is H, halo, or —OH.

30. The compound of claim 28 or claim 29, or a stereoisomer or tautomer thereof, or a pharmaceutically acceptable salt of any of the foregoing, wherein the compound is of formula (I-C3): 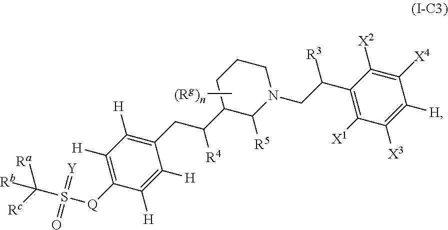
or a stereoisomer or tautomer thereof, or a pharmaceutically acceptable salt of any of the foregoing.

31. The compound of claim 30, or a stereoisomer or tautomer thereof, or a pharmaceutically acceptable salt of any of the foregoing, wherein n is 0.

32. The compound of any one of claims 1-24 and 27-31, or a stereoisomer or tautomer thereof, or a pharmaceutically acceptable salt of any of the foregoing, wherein R³, R⁴, and R⁵ are each H.

33. The compound of any one of claims 1-24 and 27-31, or a stereoisomer or tautomer thereof, or a pharmaceutically acceptable salt of any of the foregoing, wherein R³ is —OH, halo, or C₁₋₆alkoxy, R⁴ is H, and R⁵ is H.

34. The compound of claim 1 or claim 2, or a stereoisomer or tautomer thereof, or a pharmaceutically acceptable salt of any of the foregoing, wherein L is 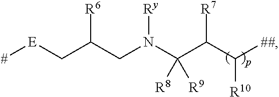
such that the compound is of formula (II): 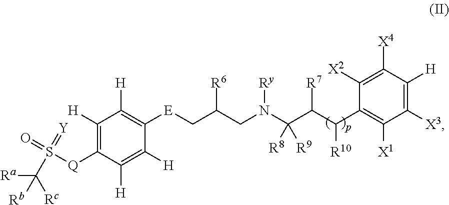
or a stereoisomer or tautomer thereof, or a pharmaceutically acceptable salt of any of the foregoing.

35. The compound of claim 34, or a stereoisomer or tautomer thereof, or a pharmaceutically acceptable salt of any of the foregoing, wherein E is O, such that the compound is of formula (II-A1): 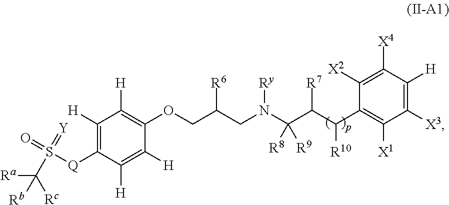
or a stereoisomer or tautomer thereof, or a pharmaceutically acceptable salt of any of the foregoing.

36. The compound of claim 35, or a stereoisomer or tautomer thereof, or a pharmaceutically acceptable salt of any of the foregoing, wherein R^{y} is taken together with R⁷, and the atoms to which they are attached, to form a 3-8 membered heterocyclyl.

37. The compound of claim 35, or a stereoisomer or tautomer thereof, or a pharmaceutically acceptable salt of any of the foregoing, wherein R^{y} is taken together with R⁷, and the atoms to which they are attached, to form a 5-6 membered heterocyclyl.

38. The compound of any one of claims 35 to 37, or a stereoisomer or tautomer thereof, or a pharmaceutically acceptable salt of any of the foregoing, wherein p is 1.

39. The compound of any one of claims 35 to 38, or a stereoisomer or tautomer thereof, or a pharmaceutically acceptable salt of any of the foregoing, wherein the compound is of formula (II-B1): 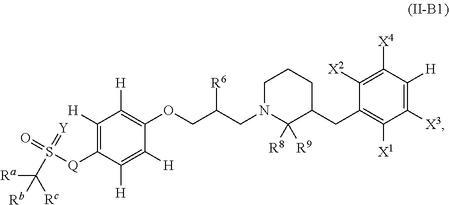
or a stereoisomer or tautomer thereof, or a pharmaceutically acceptable salt of any of the foregoing.

40. The compound of any one of claims 35 to 37, or a stereoisomer or tautomer thereof, or a pharmaceutically acceptable salt of any of the foregoing, wherein p is 0.

41. The compound of any one of claims 35 to 37 and 40, or a stereoisomer or tautomer thereof, or a pharmaceutically acceptable salt of any of the foregoing, wherein the compound is of formula (II-C1): 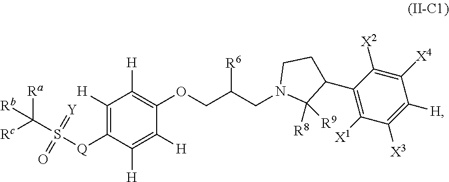
or a stereoisomer or tautomer thereof, or a pharmaceutically acceptable salt of any of the foregoing.

42. The compound of any one of claims 35 to 37, 40, and 41, or a stereoisomer or tautomer thereof, or a pharmaceutically acceptable salt of any of the foregoing, wherein R⁶ is —OH, R⁸ is C₁₋₆alkyl, and R⁹ is C₁₋₆alkyl.

43. The compound of any one of claims 35 to 37 and 40 to 42, or a stereoisomer or tautomer thereof, or a pharmaceutically acceptable salt of any of the foregoing, wherein R⁶ is —OH, R⁸ is methyl, and R⁹ is methyl.

44. The compound of any one of claims 35 to 37 and 40, or a stereoisomer or tautomer thereof, or a pharmaceutically acceptable salt of any of the foregoing, wherein the compound is of formula (II-D1): 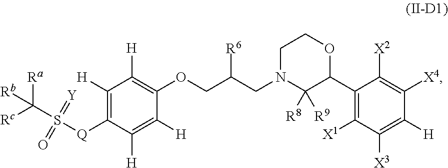
or a stereoisomer or tautomer thereof, or a pharmaceutically acceptable salt of any of the foregoing.

45. The compound of any one of claims 35 to 41 and 44, or a stereoisomer or tautomer thereof, or a pharmaceutically acceptable salt of any of the foregoing, wherein R⁶ is —OH, R⁸ is H, and R⁹ is H.

46. The compound of claim 35, or a stereoisomer or tautomer thereof, or a pharmaceutically acceptable salt of any of the foregoing, wherein one of R⁸ and R⁹ is taken together with R¹⁰, and the atoms to which they are attached, to form a C₃₋₈cycloalkyl.

47. The compound of claim 35, or a stereoisomer or tautomer thereof, or a pharmaceutically acceptable salt of any of the foregoing, wherein one of R⁸ and R⁹ is taken together with R¹⁰, and the atoms to which they are attached, to form a C₃₋₆cycloalkyl.

48. The compound of any one of claims 35, 38, 46, and 47, or a stereoisomer or tautomer thereof, or a pharmaceutically acceptable salt of any of the foregoing, wherein the compound is of formula (II-E1): 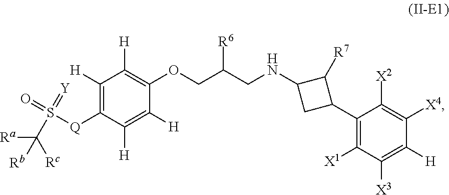
or a stereoisomer or tautomer thereof, or a pharmaceutically acceptable salt of any of the foregoing.

49. The compound of any one of claims 35, 38, 46, and 48, or a stereoisomer or tautomer thereof, or a pharmaceutically acceptable salt of any of the foregoing, wherein R⁶ is —OH and R⁷ is H.

50. The compound of claim 1 or claim 2, or a stereoisomer or tautomer thereof, or a pharmaceutically acceptable salt of any of the foregoing, wherein L is, 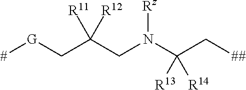
such that the compound is of formula (III): 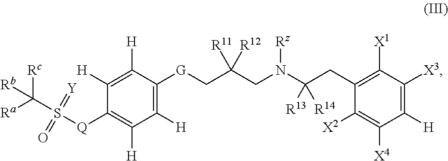
or a stereoisomer or tautomer thereof, or a pharmaceutically acceptable salt of any of the foregoing.

51. The compound of any one of claims 1 to 50, or a stereoisomer or tautomer thereof, or a pharmaceutically acceptable salt of any of the foregoing, wherein Q is absent.

52. The compound of any one of claims 1 to 51, or a stereoisomer or tautomer thereof, or a pharmaceutically acceptable salt of any of the foregoing, wherein Y is O.

53. The compound of any one of claims 1 to 51, or a stereoisomer or tautomer thereof, or a pharmaceutically acceptable salt of any of the foregoing, wherein Y is —N—(C₁₋₆alkyl).

54. The compound of any one of claims 1 to 51 and 53, or a stereoisomer or tautomer thereof, or a pharmaceutically acceptable salt of any of the foregoing, wherein Y is —N—CH₃.

55. The compound of any one of claims 1 to 50 and 52, or a stereoisomer or tautomer thereof, or a pharmaceutically acceptable salt of any of the foregoing, wherein Q is —N—(C₁₋₆alkyl).

56. The compound of any one of claims 1 to 50, 52, and 55, or a stereoisomer or tautomer thereof, or a pharmaceutically acceptable salt of any of the foregoing, wherein Q is —N—CH₃.

57. The compound of any one of claims 1 to 56, or a stereoisomer or tautomer thereof, or a pharmaceutically acceptable salt of any of the foregoing, wherein R^{a}, R^{b}, and R^{c} are each independently H.

58. The compound of any one of claims 1 to 57, or a stereoisomer or tautomer thereof, or a pharmaceutically acceptable salt of any of the foregoing, wherein X¹ is H, X² is H, one of X³ and X⁴ is halo, and the other of X³ and X⁴ is H.

59. The compound of any one of claims 1 to 58, or a stereoisomer or tautomer thereof, or a pharmaceutically acceptable salt of any of the foregoing, wherein X¹ is H, X² is H, one of X³ and X⁴ is chloro, and the other of X³ and X⁴ is H.

60. The compound of claim 1 or claim 2, or a stereoisomer or tautomer thereof, or a pharmaceutically acceptable salt of any of the foregoing, wherein Q is absent, Y is O, and R^{a}, R^{b}, and R^{c} are each independently H, such that the compound is of formula (B-2): 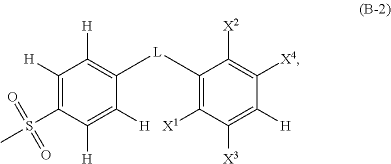
or a stereoisomer or tautomer thereof, or a pharmaceutically acceptable salt of any of the foregoing.

61. The compound of claim 1 or claim 2, or a stereoisomer or tautomer thereof, or a pharmaceutically acceptable salt of any of the foregoing, wherein Q is absent, Y is —N(CH₃), and R^{a}, R^{b}, and R^{c} are each independently H, such that the compound is of formula (B-5): 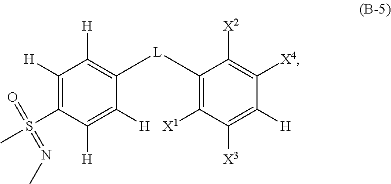
or a stereoisomer or tautomer thereof, or a pharmaceutically acceptable salt of any of the foregoing.

62. The compound of claim 1 or claim 2, or a stereoisomer or tautomer thereof, or a pharmaceutically acceptable salt of any of the foregoing, wherein Q is —N(CH₃), Y is O, and R^{a}, R^{b}, and R^{c} are each independently H, such that the compound is of formula (C-1): 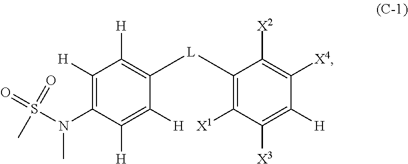
or a stereoisomer or tautomer thereof, or a pharmaceutically acceptable salt of any of the foregoing.

63. The compound of claim 1 or claim 2, or a stereoisomer or tautomer thereof, or a pharmaceutically acceptable salt of any of the foregoing, wherein the compound is a compound of Table 1, or a stereoisomer or tautomer thereof, or a pharmaceutically acceptable salt of any of the foregoing.

64. A pharmaceutical composition, comprising (i) a compound of any one of claims 1 to 63, or a stereoisomer or tautomer thereof, or a pharmaceutically acceptable salt of any of the foregoing, and (ii) one or more pharmaceutically acceptable excipients.

65. A method of modulating APOL1 in a cell, comprising exposing the cell to a composition comprising an effective amount of a compound of any one or claims 1 to 63, or a stereoisomer or tautomer thereof, or a pharmaceutically acceptable salt of any of the foregoing, or a pharmaceutical composition of claim 64.

66. A method of inhibiting APOL1 in a cell, comprising exposing the cell to a composition comprising an effective amount of a compound of any one or claims 1 to 63, or a stereoisomer or tautomer thereof, or a pharmaceutically acceptable salt of any of the foregoing, or a pharmaceutical composition of claim 64.

67. A method of treating an APOL1-mediated disease, disorder, or condition in an individual in need thereof, comprising administering to the individual a therapeutically effective amount of a compound of any one of claims 1 to 63, or a stereoisomer or tautomer thereof, or a pharmaceutically acceptable salt of any of the foregoing, or a pharmaceutical composition of claim 64.

68. The method of claim 67, wherein the disease, disorder, or condition is a kidney disease.

69. The method of claim 67 or claim 68, wherein the disease, disorder, or condition is a chronic kidney disease (CKD).

70. The method of claim 67, wherein the disease, disorder, or condition is selected from the group consisting of chronic kidney disease, focal segmental glomerulosclerosis (FSGS), hypertension-attributed kidney disease, human immunodeficiency virus-associated nephropathy (HIVAN), sickle-cell nephropathy, lupus nephritis, diabetic kidney disease, APOL1-associated nephropathy, viral nephropathy, COVID-19 associated nephropathy, preeclampsia, and sepsis.

71. A method of delaying the development of an APOL1-mediated disease, disorder, or condition, comprising administering a therapeutically effective amount of a compound of any one of claims 1 to 63, or a stereoisomer or tautomer thereof, or a pharmaceutically acceptable salt of any of the foregoing, or a pharmaceutical composition of claim 64, to an individual who is at risk of developing an APOL1-mediated disease, disorder, or condition.

72. The method of claim 71, wherein the APOL1-mediated disease, disorder, or condition is a kidney disease.

73. The method of claim 71 or claim 72, wherein the APOL1-mediated disease, disorder, or condition is a chronic kidney disease.

74. The method of claim 71, wherein the APOL1-mediated disease, disorder, or condition is selected from the group consisting of chronic kidney disease, focal segmental glomerulosclerosis (FSGS), hypertension-attributed kidney disease, human immunodeficiency virus-associated nephropathy (HIVAN), sickle-cell nephropathy, lupus nephritis, diabetic kidney disease, APOL1-associated nephropathy, viral nephropathy, COVID-19 associated nephropathy, preeclampsia, and sepsis.

75. The method of any one of claims 67 to 74, wherein the individual has an APOL1 mutation.

76. The method of claim 75, wherein the APOL1 mutation is a gain-of-function mutation.

77. A kit, comprising (i) a compound of any one of claims 1 to 63, or a stereoisomer or tautomer thereof, or a pharmaceutically acceptable salt of any of the foregoing, or a pharmaceutical composition of claim 64, and (ii) instructions for use in treating an APOL1-mediated disease, disorder, or condition in an individual in need thereof.

78. The kit of claim 77, wherein the disease, disorder, or condition is a kidney disease.

79. The kit of claim 77 or claim 78, wherein the disease, disorder, or condition is a chronic kidney disease (CKD).

80. The kit of any one of claims 77 to 79, wherein the disease, disorder, or condition is selected from the group consisting of chronic kidney disease, focal segmental glomerulosclerosis (FSGS), hypertension-attributed kidney disease, human immunodeficiency virus-associated nephropathy (HIVAN), sickle-cell nephropathy, lupus nephritis, diabetic kidney disease, APOL1-associated nephropathy, viral nephropathy, COVID-19 associated nephropathy, preeclampsia, and sepsis.

81. The kit of any one of claims 77 to 80, wherein the individual has an APOL1 mutation.

82. The kit of claim 81, wherein the APOL1 mutation is a gain-of-function mutation.
